(19) United States
(12) Patent Application Publication
(10) Pub. No.: US 2023/0106899 A1
(43) Pub. Date: Apr. 6, 2023

(54) METHODS OF TREATING CANCER

(71) Applicant: IFM Due, Inc., Boston, MA (US)

(72) Inventors: Gary Glick, Ann Arbor, MI (US); Anthony W. Opipari, Jr., Dexter, MI (US); Hans Martin Seidel, Concord, MA (US)

(21) Appl. No.: 17/619,887

(22) PCT Filed: Jun. 19, 2020
(86) PCT No.: PCT/US2020/038692

§ 371 Date: Dec. 16, 2021

Related U.S. Application Data
(60) Provisional application No. 62/865,087, filed on Jun. 21, 2019.

(51) Int. Cl. G01N 33/574; A61K 31/343; A61P 35/00; A61K 31/4035; A61K 45/06

(52) U.S. Cl. CPC G01N 33/574; A61K 31/343; A61P 35/00; A61K 31/4035; A61K 45/06

(57) ABSTRACT

Provided herein are methods of treating a subject, such as a subject that has cancer, that include administering a therapeutically effective amount of a STING antagonist or a cGAS inhibitor or a pharmaceutically acceptable salt, solvate, or co-crystal thereof to a subject identified as having (i) one or both of (i) decreased TREX1 level and/or activity, and (ii) increased cGAS/STING signaling pathway activity, e.g., as compared to a reference level, and/or (ii) an elevated level of cGAMP in a serum or tumor sample of the subject as compared to a reference level.

### CROSS REFERENCE TO RELATED APPLICATIONS

This application claims the benefit of U.S. Provisional Application Ser. No. 62/865,087, filed on Jun. 21, 2019, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jun. 19, 2020, is named sequencelisting.txt and is 433 KB in size.

### TECHNICAL FIELD

The present disclosure relates to, in part, methods of treating a subject, e.g., a subject having cancer, which include administration of a STING antagonist or a cGAS inhibitor.

### BACKGROUND

The cGAS/STING (cyclic GMP-AMP Synthase/Stimulator of Interferon Genes) pathway is a component of inflammatory signaling pathways. When DNA is present in the cytosol of a cell, cGAS binds it and generates 2′-5′ cyclic GMP-AMP (cGAMP). Activated by cGAMP, STING induces the phosphorylation of and nuclear translocation of interferon (IFN) regulatory factors (IRFs). As transcription factors, IRFs regulate the expression of genes, including the type I IFNs, which regulate the activity of the immune system.

The presence of DNA in the cytosol of a cell can sometimes be the result of an infection. In some cases, the presence of DNA in the cytosol of a cell can be the result of DNA damage in the nucleus of a cell or in the mitochondria of a cell. In some instances, the cytosolic DNA is degraded or modified by enzymes to prevent activation of the cGAS/STING pathway. One such enzyme is TREX1 (three-prime repair exonuclease 1; also called DNaseIII).

### SUMMARY

The present disclosure is based on the discovery that cancer cells having decreased TREX1 level and/or activity and/or increased cGAS/STING signaling pathway activity and/or an elevated level of cGAMP are more sensitive to treatment with a STING antagonist or a cGAS inhibitor, e.g., than cells that do not have decreased TREX1 level and/or activity and/or increased cGAS/STING signaling pathway activity.

Provided herein are methods of treating a subject in need thereof that include: (a) identifying a subject having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level; and (b) administering a treatment including a therapeutically effective amount of a STING antagonist or a cGAS inhibitor, or a pharmaceutically acceptable salt, solvate, or co-crystal thereof to the identified subject.

Also provided herein are methods of treating a subject in need thereof that include administering a treatment including a therapeutically effective amount of a STING antagonist or acGAS inhibitor, or a pharmaceutically acceptable salt, solvate, or co-crystal thereof to a subject identified as having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level.

Also provided herein are methods of selecting a treatment for a subject in need thereof that include: (a) identifying a subject having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level; and (b) selecting for the identified subject a treatment including a therapeutically effective amount of a STING antagonist, or cGAS inhibitor, or a pharmaceutically acceptable salt, solvate, or co-crystal thereof.

Also provided herein are methods of selecting a treatment for a subject in need thereof that include: selecting a treatment including a therapeutically effective amount of a STING antagonist or cGAS inhibitor, or a pharmaceutically acceptable salt, solvate, or co-crystal thereof for a subject identified as having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level.

Also provided herein are methods of selecting a subject for treatment that include: (a) identifying a subject having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level; and (b) selecting the identified subject for treatment with a therapeutically effective amount of a STING antagonist or cGAS inhibitor, or a pharmaceutically acceptable salt, solvate, or co-crystal thereof.

Also provided herein are methods of selecting a subject for participation in a clinical trial that include: (a) identifying a subject having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level; and (b) selecting the identified subject for participation in a clinical trial that includes administration of a treatment including a therapeutically effective amount of a STING antagonist or cGAS inhibitor, or a pharmaceutically acceptable salt, solvate, or co-crystal thereof.

Also provided herein of selecting a subject for participation in a clinical trial that include selecting a subject identified as having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level, for participation in a clinical trial that includes administration of a treatment including a therapeutically effective amount of a STING antagonist or cGAS inhibitor, or a pharmaceutically acceptable salt, solvate, or co-crystal thereof.

Also provided herein are methods of predicting a subject's responsiveness to a STING antagonist or cGAS inhibitor that include: (a) determining that a subject has a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level; and (b) identifying that the subject determined to have (i) one or both of (i) decreased TREX1 expression and/or activity, and (ii) increased cGAS/STING signaling pathway activity and/or (ii) an elevated level of cGAMP in a serum or tumor sample of the subject as compared to a reference level, in step (a) has an increased likelihood of being responsive to treatment with a STING antagonist or cGAS inhibitor.

Also provided herein are methods of predicting a subject's responsiveness to a STING antagonist or cGAS inhibitor that include identifying a subject determined to have a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level as having an increased likelihood of being responsive to treatment with a STING antagonist or cGAS inhibitor.

In some embodiments of any of the methods described herein, the subject is identified as having a cancer cell having decreased TREX1 level and/or activity. In some embodiments of any of the methods described herein, the subject is identified as having a cancer cell having increased cGAS/STING signaling pathway activity. In some embodiments of any of the methods described herein, the subject is identified having a cancer cell having both (i) decreased TREX1 level and/or activity and (ii) increased cGAS/STING signaling pathway activity.

In some embodiments of any of the methods described herein, the subject is identified as having a cancer cell having decreased TREX1 level. In some embodiments of any of the methods described herein, the TREX1 level is a level of TREX1 protein in the cancer cell. In some embodiments of any of the methods described herein, the identification of the subject as having a cancer cell having a decreased TREX1 level includes detecting a decreased level of TREX1 protein in the cancer cell. In some embodiments of any of the methods described herein, the TREX1 level is a level of TREX1 mRNA in the cancer cell. In some embodiments of any of the methods described herein, the identification of the subject as having a cancer cell having a decreased TREX1 level includes detecting a decreased level of TREX1 mRNA in the cancer cell.

In some embodiments of any of the methods described herein, the decreased TREX1 level and/or activity is a result of TREX1 gene loss in the cancer cell. In some embodiments of any of the methods described herein, the TREX1 gene loss is loss of one allele of the TREX1 gene. In some embodiments of any of the methods described herein, the TREX1 gene loss is loss of both alleles of the TREX1 gene. In some embodiments of any of the methods described herein, the identification of the subject as having a cancer cell having decreased TREX1 level and/or activity includes detecting TREX1 gene loss in the cancer cell.

In some embodiments of any of the methods described herein, the decreased TREX1 level and/or activity is a result of one or more amino acid deletions in a protein encoded by a TREX1 gene in the cancer cell. In some embodiments of any of the methods described herein, the identification of the subject as having a cancer cell having decreased TREX1 level and/or activity includes detecting one or more amino acid deletions in a protein encoded by a TREX1 gene in the cancer cell. In some embodiments of any of the methods described herein, the decreased TREX1 level and/or activity is a result of one or more inactivating amino acid substitutions in a protein encoded by a TREX1 gene in the cancer cell. In some embodiments of any of the methods described herein, the identification of the subject as having a cancer cell having decreased TREX1 expression and/or activity includes detecting one or more inactivating amino acid substitutions in a protein encoded by a TREX1 gene in the cancer cell.

In some embodiments of any of the methods described herein, the increased cGAS/STING signaling pathway activity is a result of a decreased level and/or activity of BRCA1 in the cancer cell. In some embodiments of any of the methods described herein, the decreased level and/or activity of BRCA1 in the cancer cell is a result of a frameshift mutation in a BRCA1 gene. In some embodiments of any of the methods described herein, the frameshift mutation in a BRCA1 gene is a E111Gfs*3 frameshift insertion. In some embodiments of any of the methods described herein, the decreased level and/or activity of BRCA1 in the cancer cell is a result of BRCA1 gene loss in the cancer cell. In some embodiments of any of the methods described herein, the decreased level and/or activity of BRCA1 in the cancer cell is a result of one or more amino acid deletions in a protein encoded by a BRCA1 gene. In some embodiments of any of the methods described herein, the decreased level and/or activity of BRCA1 in the cancer cell is a result of one or more inactivating amino acid substitutions in a protein encoded by a BRCA1 gene.

In some embodiments of any of the methods described herein, the increased cGAS/STING signaling pathway activity is a result of a decreased level and/or activity of BRCA2 gene. In some embodiments of any of the methods described herein, the decreased level and/or activity of BRCA2 in the cancer cell is a result of a frameshift mutation in a BRCA2 gene. In some embodiments of any of the methods described herein, the frameshift mutation in a BRCA2 gene is a N1784Kfs*3 frameshift insertion. In some embodiments of any of the methods described herein, the decreased level and/or activity of BRCA2 in the cancer cell is a result of BRCA2 gene loss in the cancer cell. In some embodiments of any of the methods described herein, the decreased level and/or activity of BRCA2 in the cancer cell is a result of one or more amino acid deletions in a protein encoded by a BRCA2 gene. In some embodiments of any of the methods described herein, the decreased level and/or activity of BRCA2 in the cancer cell is a result of one or more inactivating amino acid substitutions in a protein encoded by a BRCA2 gene.

In some embodiments of any of the methods described herein, the increased cGAS/STING signaling pathway activity is a result of a decreased level and/or activity of SAMHD1 in the cancer cell. In some embodiments of any of the methods described herein, the decreased level and/or activity of SAMHD1 in the cancer cell is a result of one or more inactivating amino acid substitutions in a protein encoded by a SAMHD1 gene in the cancer cell. In some embodiments of any of the methods described herein, the one or more inactivating amino acid substitutions in a protein encoded by a SAMHD1 gene is a V133I amino acid substitution. In some embodiments of any of the methods described herein, the decreased level and/or activity of SAMHD1 in the cancer cell is a result of SAMHD1 gene loss in the cancer cell. In some embodiments of any of the methods described herein, the decreased level and/or activity of SAMHD1 in the cancer cell is a result of one or more amino acid deletions in a protein encoded by a SAMHD1 gene.

In some embodiments of any of the methods described herein, the increased cGAS/STING signaling pathway activity is a result of a decreased level and/or activity of DNASE2 in the cancer cell. In some embodiments of any of the methods described herein, the decreased level and/or activity of DNASE2 in the cancer cell is a result of one or more inactivating amino acid substitutions in a protein encoded by a DNASE2 gene in the cancer cell. In some embodiments of any of the methods described herein, the one or more inactivating amino acid substitutions in a protein encoded by a DNASE2 gene is a R314W amino acid substitution. In some embodiments of any of the methods described herein, the decreased level and/or activity of DNASE2 in the cancer cell is a result of DNASE2 gene loss in the cancer cell. In some embodiments of any of the methods described herein, the decreased level and/or activity of DNASE2 in the cancer cell is a result of one or more amino acid deletions in a protein encoded by a DNASE2 gene.

In some embodiments of any of the methods described herein, the increased cGAS/STING signaling pathway activity is a result of a decreased level and/or activity of BLM in the cancer cell. In some embodiments of any of the methods described herein, the decreased level and/or activity of BLM in the cancer cell is a result of a frameshift mutation in a BLM gene. In some embodiments of any of the methods described herein, the frameshift mutation in a BLM gene is a N515Mfs*16 frameshift deletion. In some embodiments of any of the methods described herein, the decreased level and/or activity of BLM in the cancer cell is a result of BLM gene loss in the cancer cell. In some embodiments of any of the methods described herein, the decreased level and/or activity of BLM in the cancer cell is a result of one or more amino acid deletions in a protein encoded by a BLM gene. In some embodiments of any of the methods described herein, the decreased level and/or activity of BLM in the cancer cell is a result of one or more inactivating amino acid substitutions in a protein encoded by a BLM gene.

In some embodiments of any of the methods described herein, the increased cGAS/STING signaling pathway activity is a result of a decreased level and/or activity of PARP1 in the cancer cell. In some embodiments of any of the methods described herein, the decreased level and/or activity of PARP1 in the cancer cell is a result of a frameshift mutation in a PARP1 gene. In some embodiments of any of the methods described herein, the frameshift mutation in a PARP1 gene is a S507Afs*17 frameshift deletion. In some embodiments of any of the methods described herein, the decreased level and/or activity of PARP1 in the cancer cell is a result of PARP1 gene loss in the cancer cell. In some embodiments of any of the methods described herein, the decreased level and/or activity of PARP1 in the cancer cell is a result of one or more amino acid deletions in a protein encoded by a PARP1 gene. In some embodiments of any of the methods described herein, the decreased level and/or activity of PARP1 in the cancer cell is a result of one or more inactivating amino acid substitutions in a protein encoded by a PARP1 gene.

In some embodiments of any of the methods described herein, the increased cGAS/STING signaling pathway activity is a result of a decreased level and/or activity of RPA1 in the cancer cell. In some embodiments of any of the methods described herein, the decreased level and/or activity of RPA1 in the cancer cell is a result of a mutation that results in aberrant RPA1 mRNA splicing in the cancer cell. In some embodiments of any of the methods described herein, the mutation that results in aberrant RPA1 mRNA splicing in the cancer cell is a X12 splice mutation. In some embodiments of any of the methods described herein, the decreased level and/or activity of RPA1 in the cancer cell is a result of RPA1 gene loss in the cancer cell. In some embodiments of any of the methods described herein, the decreased level and/or activity of RPA1 in the cancer cell is a result of one or more amino acid deletions in a protein encoded by a RPA1 gene. In some embodiments of any of the methods described herein, the decreased level and/or activity of RPA1 in the cancer cell is a result of one or more inactivating amino acid substitutions in a protein encoded by a RPA1 gene.

In some embodiments of any of the methods described herein, the increased cGAS/STING signaling pathway activity is a result of a decreased level and/or activity of RAD51 in the cancer cell. In some embodiments of any of the methods described herein, the decreased level and/or activity of RAD51 in the cancer cell is a result of one or more inactivating amino acid substitutions in a protein encoded by a RAD51 gene. In some embodiments of any of the methods described herein, the one or more inactivating amino acid substitutions in a protein encoded by a RAD51 gene is an R254* amino acid substitution. In some embodiments of any of the methods described herein, the decreased level and/or activity of RAD51 in the cancer cell is a result of RAD51 gene loss in the cancer cell. In some embodiments of any of the methods described herein, the decreased level and/or activity of RAD51 in the cancer cell is a result of one or more amino acid deletions in a protein encoded by a RAD51 gene.

In some embodiments of any of the methods described herein, the increased cGAS/STING signaling pathway activity is a result of an increased level and/or activity of MUS81 in the cancer cell. In some embodiments of any of the methods described herein, the increased level and/or activity of MUS81 in the cancer cell is a result of MUS81 gene amplification in the cancer cell. In some embodiments of any of the methods described herein, the increased level and/or activity of MUS81 in the cancer cell is a result of one or more activating amino acid substitutions in a protein encoded by a MUS81 gene.

In some embodiments of any of the methods described herein, the increased cGAS/STING signaling pathway activity is a result of an increased level and/or activity of IFI16 in the cancer cell. In some embodiments of any of the methods described herein, the increased level and/or activity of IFI16 in the cancer cell is a result of IFI16 gene amplification in the cancer cell. In some embodiments of any of the methods described herein, the increased level and/or activity of IFI16 in the cancer cell is a result of one or more activating amino acid substitutions in a protein encoded by a IFI16 gene.

In some embodiments of any of the methods described herein, the increased cGAS/STING signaling pathway activity is a result of an increased level and/or activity of cGAS in the cancer cell. In some embodiments of any of the methods described herein, the increased level and/or activity of cGAS in the cancer cell is a result of cGAS gene amplification in the cancer cell. In some embodiments of any of the methods described herein, the increased level and/or activity of cGAS in the cancer cell is a result of one or more activating amino acid substitutions in a protein encoded by a cGAS gene.

In some embodiments of any of the methods described herein, the increased cGAS/STING signaling pathway activity is a result of an increased level and/or activity of DDX41 in the cancer cell. In some embodiments of any of the methods described herein, the increased level and/or activity of DDX41 in the cancer cell is a result of DDX41 gene amplification in the cancer cell. In some embodiments of any of the methods described herein, the increased level and/or activity of DDX41 in the cancer cell is a result of one or more activating amino acid substitutions in a protein encoded by a DDX41 gene.

In some embodiments of any of the methods described herein, the increased cGAS/STING signaling pathway activity is a result of an increased level and/or activity of EXO1 in the cancer cell. In some embodiments of any of the methods described herein, the increased level and/or activity of EXO1 in the cancer cell is a result of EXO1 gene amplification in the cancer cell. In some embodiments of any of the methods described herein, the increased level and/or activity of EXO1 in the cancer cell is a result of one or more activating amino acid substitutions in a protein encoded by a EXO1 gene.

In some embodiments of any of the methods described herein, the increased cGAS/STING signaling pathway activity is a result of an increased level and/or activity of DNA2 in the cancer cell. In some embodiments of any of the methods described herein, the increased level and/or activity of DNA2 in the cancer cell is a result of DNA2 gene amplification in the cancer cell. In some embodiments of any of the methods described herein, the increased level and/or activity of DNA2 in the cancer cell is a result of one or more activating amino acid substitutions in a protein encoded by a DNA2 gene.

In some embodiments of any of the methods described herein, the increased cGAS/STING signaling pathway activity is a result of an increased level and/or activity of RBBP8 (CtIP) in the cancer cell. In some embodiments of any of the methods described herein, the increased level and/or activity of RBBP8 (CtIP) in the cancer cell is a result of RBBP8 (CtIP) gene amplification in the cancer cell. In some embodiments of any of the methods described herein, the increased level and/or activity of RBBP8 (CtIP) in the cancer cell is a result of one or more activating amino acid substitutions in a protein encoded by a RBBP8 (CtIP) gene.

In some embodiments of any of the methods described herein, the increased cGAS/STING signaling pathway activity is a result of an increased level and/or activity of MRE11 in the cancer cell. In some embodiments of any of the methods described herein, the increased level and/or activity of MRE11 in the cancer cell is a result of MRE11 gene amplification in the cancer cell. In some embodiments of any of the methods described herein, the increased level and/or activity of MRE11 in the cancer cell is a result of one or more activating amino acid substitutions in a protein encoded by a MRE11 gene.

Some embodiments of any of the methods described herein further include administering the selected treatment to the subject. Some embodiments of any of the methods described herein further include administering a therapeutically effective amount of a STING antagonist or cGAS inhibitor to a subject identified as having an increased likelihood of being responsive to treatment with a STING antagonist or cGAS inhibitor.

In some embodiments of any of the methods described herein, the subject has been diagnosed or identified as having a cancer. In some embodiments of any of the methods described herein, the cancer is selected from the group of: renal clear cell carcinoma, uveal melanoma, tongue squamous cell carcinoma, breast cancer, and skin cancer.

In some embodiments of any of the methods described herein, the STING antagonist or cGAS inhibitor is a compound of any one of Formulas I-X, or a pharmaceutically acceptable salt, solvate, or co-crystal thereof. In some embodiments of any of the methods described herein, the STING antagonist or cGAS inhibitor is a compound selected from the group consisting of the compounds in Tables 1-10, or a pharmaceutically acceptable salt, solvate, or co-crystal thereof.

As used herein, the term “STING antagonist” is an agent that decreases one or both of (i) the activity of STING (e.g., any of the exemplary activities of STING described herein) (e.g., as compared to the level of STING activity in the absence of the agent) and (ii) the expression level of STING in a mammalian cell (e.g., using any of the exemplary methods of detection described herein) (e.g., as compared to the expression level of STING in a mammalian cell not contacted with the agent). Non-limiting examples of STING antagonists are described herein.

As used herein, the term “STING” is meant to include, without limitation, nucleic acids, polynucleotides, oligonucleotides, sense and antisense polynucleotide strands, complementary sequences, peptides, polypeptides, proteins, homologous and/or orthologous STING molecules, isoforms, precursors, mutants, variants, derivatives, splice variants, alleles, different species, and active fragments thereof.

As used herein, the term “cGAS inhibitor” is an agent that decreases one or both of (i) the activity of cGAS (e.g., any of the exemplary activities of cGAS described herein) (e.g., as compared to the level of cGAS activity in the absence of the agent) and (ii) the expression level of cGAS in a mammalian cell (e.g., using any of the exemplary methods of detection described herein) (e.g., as compared to the expression level of cGAS in a mammalian cell not contacted with the agent). Non-limiting examples of cGAS inhibitors are described herein.

As used herein, the term “cGAS” is meant to include, without limitation, nucleic acids, polynucleotides, oligonucleotides, sense and antisense polynucleotide strands, complementary sequences, peptides, polypeptides, proteins, homologous and/or orthologous cGAS molecules, isoforms, precursors, mutants, variants, derivatives, splice variants, alleles, different species, and active fragments thereof.

The term “acceptable” with respect to a formulation, composition, or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a STING antagonist or cGAS inhibitor being administered that will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a STING antagonist or cGAS inhibitor disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “pharmaceutically acceptable salt” may refer to pharmaceutically acceptable addition salts prepared from pharmaceutically acceptable non-toxic acids including inorganic and organic acids. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. The term “pharmaceutically acceptable salt” may also refer to pharmaceutically acceptable addition salts prepared by reacting a compound having an acidic group with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salts not specifically limited as far as it can be used in medicaments. Examples of a salt that the compounds described herein from with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a STING antagonist or cGAS inhibitor with other chemical components (referred to collectively herein as “excipients”), such as carriers, stabilizers, diluents, dispersing agents, suspending agents, and/or thickening agents. The pharmaceutical composition facilitates administration of the STING antagonist or cGAS inhibitor to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human. In some embodiments of any of the methods described herein, the subject is 1 year old or older, 2 years old or older, 4 years old or older, 5 years old or older, 10 years old or older, 12 years old or older, 13 years old or older, 15 years old or older, 16 years old or older, 18 years old or older, 20 years old or older, 25 years old or older, 30 years old or older, 35 years old or older, 40 years old or older, 45 years old or older, 50 years old or older, 55 years old or older, 60 years old or older, 65 years old or older, 70 years old or older, 75 years old or older, 80 years old or older, 85 years old or older, 90 years old or older, 95 years old or older, 100 years old or older, or 105 years old or older,

In some embodiments of any of the methods described herein, the subject has been previously diagnosed or identified as having a disease associated with STING activity (e.g., a cancer, e.g., any of the exemplary types of cancer described herein). In some embodiments of any of the methods described herein, the subject is suspected of having a cancer (e.g., any of the exemplary cancers described herein). In some embodiments of any of the methods described herein, the subject is presenting with one or more (e.g., two, three, four, or five) symptoms of a cancer (e.g., any of the exemplary cancers described herein).

In some embodiments of any of the methods described herein, the subject is a participant in a clinical trial. In some embodiments of any of the methods described herein, the subject has been previously administered a pharmaceutical composition and the different pharmaceutical composition was determined not to be therapeutically effective.

The term “administration” or “administering” refers to a method of providing a dosage of a pharmaceutical composition or a compound to an invertebrate or a vertebrate, including a fish, a bird and a mammal (e.g., a human). In some aspects, administration is performed, e.g., orally, intravenously, subcutaneously, intranasally, transdermally, intraperitoneally, intramuscularly, intrapulmonarilly, intralymphatic, topically, intraocularly, vaginally, rectally, intrathecally, or intracystically. The method of administration can depend on various factors, e.g., the site of the disease, the severity of the disease, and the components of the pharmaceutical composition.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread, or worsening of a disease, disorder or condition or of one or more symptoms thereof.

The phrase “an elevated level” or “an increased level” as used herein can be an increase or 1.1× to 100×, or higher (such as up to 200×) e.g., as compared to a reference level (e.g., any of the exemplary reference levels described herein). In some aspects, “an elevated level” or “an increased level” can be an increase of at least 1% (e.g., at least 2%, at least 4, at least 6%, at least 8%, at least 10%, at least 12%, at least 14%, at least 16%, at least 18%, at least 20%, at least 22%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 110%, at least 120%, at least 130%, at least 140%, at least 150%, at least 160%, at least 170%, at least 180%, at least 190%, at least 200%, at least 220%, at least 250%, at least 280%, at least 300%, at least 320%, at least 350%, at least 380%, at least 400%, at least 420%, at least 450%, at least 480%, at least 500%, at least 600%, at least 700%, at least 800%, at least 900%, or at least 1000%), e.g., as compared to a reference level (e.g., any of the exemplary reference levels described herein).

The phrase “a decreased level” as used herein can be a decrease of at least 1% (e.g., at least 2%, at least 4, at least 6%, at least 8%, at least 10%, at least 12%, at least 14%, at least 16%, at least 18%, at least 20%, at least 22%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, e.g., as compared to a reference level (e.g., any of the exemplary reference levels described herein).

The phrase “decreased level of TREX1” means a decrease in the level of TREX1 protein and/or TREX1 mRNA in a mammalian cell. For example, a decrease in the level of TREX1 can be a result of a TREX1 gene loss (at one or both alleles), an mutation in a regulatory region of a TREX1 gene that results in decreased transcription of a TREX1 gene, or a mutation that results in the production of a TREX1 protein that has decreased stability and/or half-life in a mammalian cell.

The phrase “protein activity” (or “activity” of a particular protein) means one or more activities of the protein (e.g., enzymatic activity, localization activity, binding activity (e.g., binding another protein or binding a non-protein (e.g., a nucleic acid)). A decrease in activity of a protein in a mammalian cell can be, e.g., the result of an amino acid deletion in the protein, or an amino acid substitution in the protein, e.g., as compared to the wildtype protein. In some cases, an increase in activity of a protein in a mammalian cell can be, e.g., the result of gene amplification or an activating amino acid substitution in the protein, e.g., as compared to the wildtype protein.

The phrase “TREX1 activity” means 3′-exonuclease activity. For example, a decrease in TREX1 activity in a mammalian cell can be the result of, e.g., TREX1 gene loss (e.g., at one or both alleles), one or more nucleotide substitutions, deletions, and/or insertions in the TREX1 gene, one or more amino acid deletions, substitutions, insertions, truncations, or other modifications to the protein sequence of TREX1 protein, or one or more post-translational modifications to TREX1 protein that alter its activity, localization or function.

The term “increased STING pathway activity” means an increase in direct activity of STING in a mammalian cell (e.g., translocation of STING from the endoplasmic reticulum to the perinuclear area, or activation of TBK1 (TANK Binding Kinase 1); or an increase in upstream activity or a mutation (e.g., any of the exemplary mutations or single nucleotide polymorphisms described herein) in a mammalian cell that results in increased STING pathway activity in the mammalian cell (e.g., decreased level or activity of one or more of BRCA1, BRCA2, SAMHD1, DNASE2, BLM, PARP1, RPA1, and RAD51 (e.g., as compared to any of the exemplary reference levels described herein) or increased level or activity of one or more of MUS81, IFI16, cGAS, DDX41, EXO1, DNA2, RBBP8, and MRE11 (e.g., as compared to any of the exemplary reference levels described herein).

A decreased level or activity of one or more of BRCA1, BRCA2, SAMHD1, DNASE2, BLM, PARP1, RPA1, and RAD51 (e.g., in a cancer cell) can be caused by any mechanism.

In some embodiments, a decreased level or activity of BRCA1 can be a result of a frameshift mutation in a BRCA1 gene (e.g., an E111Gfs*3 frameshift insertion). In some embodiments, a decreased level or activity of BRCA1 can be a result of a BRCA1 gene loss (e.g., loss of one allele of BRCA1 or loss of both alleles of BRCA1). In some embodiments, a decreased level or activity of BRCA1 can be a result of one or more amino acid deletions in a protein encoded by a BRCA1 gene. In some embodiments, a decreased level or activity of BRCA1 in a can be a result of one or more inactivating amino acid substitutions in a protein encoded by a BRCA1 gene.

In some embodiments, a decreased level or activity of a BRCA2 gene can be result of a frameshift mutation in a BRCA2 gene (e.g., a N1784Kfs*3 frameshift insertion). In some embodiments, a decreased level or activity of BRCA2 can be a result of BRCA2 gene loss (e.g., loss of one allele of BRCA2 or loss of both alleles of BRCA2). In some embodiments, a decreased level or activity of BRCA2 can be a result of one or more amino acid deletions in a protein encoded by a BRCA2 gene. In some embodiments, a decreased level or activity of BRCA2 can be a result of one or more inactivating amino acid substitutions in a protein encoded by a BRCA2 gene.

In some embodiments, a decreased level or activity of SAMHD1 can be a result of one or more inactivating amino acid substitutions in a protein encoded by a SAMHD1 gene (e.g., a V133I amino acid substitution). In some embodiments, a decreased level or activity of SAMHD1 can be a result of gene loss (e.g., loss of one allele of SAMHD1 or loss of both alleles of SAMHD1). In some embodiments, a decreased level or activity of SAMHD1 can be a result of one or more amino acid deletions in a protein encoded by a SAMHD1 gene.

In some embodiments, a decreased level or activity of DNASE2 can be a result of one or more inactivating mutations in a protein encoded by a DNASE2 gene (e.g., a R314W amino acid substitution). In some embodiments, a decreased level or activity of DNASE2 can be a result of DNASE2 gene loss (e.g., loss of one allele of DNASE2 or loss of both alleles of DNASE2). In some embodiments, a decreased level or activity of DNASE2 can be a result of one or more amino acid deletions in a protein encoded by a DNASE2 gene.

In some embodiments, a decreased level or activity of BLM can be a result of a frameshift mutation in a BLM gene (e.g., a N515Mfs*16 frameshift deletion). In some embodiments, a decreased level or activity of BLM can be a result of BLM gene loss (e.g., loss of one allele of BLM or loss of both alleles of BLM). In some embodiments, a decreased level or activity of BLM can be a result of one or more amino acid deletions in a protein encoded by a BLM gene. In some embodiments, a decreased level or activity of BLM can be a result of one or more inactivating amino acid substitutions in a protein encoded by a BLM gene.

In some embodiments, a decreased level or activity of PARP1 can be a result of a frameshift mutation in a PARP1 gene (e.g., a S507Afs*17 frameshift deletion). In some embodiments, a decreased level or activity of PARP1 can be a result of gene loss (e.g., loss of one allele of PARP1 or loss of both alleles of PARP1). In some embodiments, a decreased level or activity of PARP1 can be a result of one or more amino acid deletions in a protein encoded by a PARP1 gene. In some embodiments, a decreased level or activity of PARP1 can be a result of one or more inactivating amino acid substitutions in a protein encoded by a PARP1 gene.

In some embodiments, a decreased level or activity of RPA1 can be a result of a mutation that results in aberrant RPA mRNA splicing (e.g., a X12 splice mutation). In some embodiments, a decreased level or activity of RPA1 can be a result of RPA1 gene loss (e.g., loss of one allele of RPA1 or loss of both alleles of RPA1). In some embodiments, a decreased level or activity of RPA1 can be a result of one or more amino acid deletions in a protein encoded by a RPA1 gene. In some embodiments, a decreased level or activity of RPA1 can be a result of one or more inactivating amino acid substitutions in a protein encoded by a RPA1 gene.

In some embodiments, a decreased level or activity of RAD51 can be a result of one or more inactivating mutations in a protein encoded by a RAD51 gene (e.g., a R254* mutation). In some embodiments, a decreased level or activity of RAD51 can be a result of RAD51 gene loss (e.g., loss of one allele of RAD51 or loss of both alleles of RAD51). In some embodiments, a decreased level or activity of RAD51 can be a result of one or more amino acid deletions in a protein encoded by a RAD51 gene.

An increased level or activity of one or more of MUS81, IFI16, cGAS, DDX41, EXO1, DNA2, RBBP8, or MRE11 (e.g., in a cancer cell) can be caused by any mechanism.

In some embodiments, an increased level or activity of MUS81 can be a result of MUS81 gene amplification. In some embodiments, an increased level or activity of MUS81 can be a result of one or more activating amino acid substitutions in a protein encoded by a MUS81 gene.

In some embodiments, an increased level or activity of IFI16 can be a result of IFI16 gene amplification. In some embodiments, an increased level or activity of IFI16 can be a result of one or more activating amino acid substitutions in a protein encoded by an IFI16 gene.

In some embodiments, an increased level or activity of cGAS can be a result of cGAS gene amplification. In some embodiments, an increased level or activity of cGAS can be a result of one or more activating amino acid substitutions in a protein encoded by a cGAS gene.

In some embodiments, an increased level or activity of DDX41 can be a result of DDX41 gene amplification. In some embodiments, an increased level or activity of DDX41 can be a result of one or more activating amino acid substitutions in a protein encoded by a DDX41 gene.

In some embodiments, an increased level or activity of EXO1 can be a result of EXO1 gene amplification. In some embodiments, an increased level or activity of EXO1 can be a result of one or more activating amino acid substitutions in a protein encoded by an EXO1 gene.

In some embodiments, an increased level or activity of DNA2 can be a result of DNA2 gene amplification. In some embodiments, an increased level or activity of DNA2 can be a result of one or more activating amino acid substitutions in a protein encoded by a DNA2 gene.

In some embodiments, an increased level or activity of RBBP8 (also called CtIP) can be a result of RBBP8 gene amplification. In some embodiments, an increased level or activity of RBBP8 can be a result of one or more activating amino acid substitutions in a protein encoded by a RBBP8 gene.

In some embodiments, an increased level or activity of MRE11 can be a result of MRE11 gene amplification. In some embodiments, an increased level or activity of MRE11 can be a result of one or more activating amino acid substitutions in a protein encoded by a MRE11 gene.

Non-limiting examples of human protein and human cDNA sequences for STING, TREX1, BRCA1, BRCA2, SAMHD1, DNASE2, BLM, PARP1, RPA1, RAD51, MUS81, IFI16, cGAS, DDX41, EXO1, DNA2, RBBP8 (CtIP), and MRE11 are shown below (SEQ ID NOs.: 1-89). It will be understood that other natural variants of these sequences can exist, and it will be understood that the name of a gene can be used to refer to the gene or to its protein product.

| SEQUENCE NAME | SEQ ID NO: |
|---|---|
| Human STING cDNA, Variant 1 | 1 |
| Human STING Protein, Variant 1 | 2 |
| Human STING cDNA, Variant 2 | 3 |
| Human STING Protein, Variant 2 | 4 |
| Human STING cDNA, Variant 3 Precursor | 5 |
| HUMAN STING Protein, Variant 3 Precursor | 6 |
| Human STING cDNA, Variant 3 Mature Sequence | 7 |
| HUMAN STING Protein, Variant 3 Mature Sequence | 8 |
| Human TREX1 cDNA Sequence, Variant 1 | 9 |
| Human TREX1 Protein Sequence, Variant 1 | 10 |
| Human TREX1 cDNA Sequence, Variant 2 | 11 |
| Human TREX1 Protein Sequence, Variant 2 | 12 |
| Human TREX Protein Sequence, Variant 3 | 13 |
| Human BRCA1 cDNA Sequence, Variant 1 | 14 |
| Human BRCA1 Protein Sequence, Variant 1 | 15 |
| Human BRCA1 cDNA Sequence, Variant 2 | 16 |
| Human BRCA1 Protein Sequence, Variant 2 | 17 |
| Human BRCA1 cDNA Sequence, Variant 3 | 18 |
| Human BRCA1 Protein Sequence, Variant 3 | 19 |
| Human BRCA1 cDNA Sequence, Variant 4 | 20 |
| Human BRCA1 Protein Sequence, Variant 4 | 21 |
| Human BRCA1 cDNA Sequence, Variant 5 | 22 |
| Human BRCA1 Protein Sequence, Variant 5 | 23 |
| Human BRCA2 cDNA Sequence | 24 |
| Human BRCA2 Protein Sequence | 25 |
| Human SAMHD1 cDNA Sequence, Variant 1 | 26 |
| Human SAMHD1 Protein Sequence, Variant 1 | 27 |
| Human SAMHD1 cDNA Sequence, Variant 2 | 28 |
| Human SAMHD1 Protein Sequence, Variant 2 | 29 |
| Human SAMHD1 cDNA Sequence, Variant 3 | 30 |
| Human SAMHD1 Protein Sequence, Variant 3 | 31 |
| Human DNASE2 Precursor cDNA Sequence | 32 |
| Human DNASE2 Precursor Protein Sequence | 33 |
| Human DNASE2 Mature cDNA Sequence | 34 |
| Human DNASE2 Mature Protein Sequence | 35 |
| Human BLM cDNA Sequence, Variant 1 | 36 |
| Human BLM Protein Sequence, Variant 1 | 37 |
| Human BLM cDNA Sequence, Variant 2 | 38 |
| Human BLM Protein Sequence, Variant 2 | 39 |
| Human BLM cDNA Sequence, Variant 3 | 40 |
| HUMAN BLM Protein Sequence, Variant 3 | 41 |
| Human PARP1 cDNA sequence | 42 |
| Human PARP protein sequence | 43 |
| Human RPA1 cDNA Sequence, Variant 1 | 44 |
| Human RPA1 Protein Sequence, Variant 1 | 45 |
| Human RPA1 cDNA Sequence, Variant 2 | 46 |
| HUMAN RPA1 Protein Sequence, Variant 2 | 47 |
| Human RPA1 cDNA Sequence, Variant 3 | 48 |
| HUMAN RPA1 Protein Sequence, Variant 3 | 49 |
| Human RAD51 cDNA Sequence, Variant 1 | 50 |
| Human RAD51 Protein Sequence, Variant 1 | 51 |
| Human RAD51 cDNA Sequence, Variant 2 | 52 |
| Human RAD51 Protein Sequence, Variant 2 | 53 |
| Human RAD51 cDNA Sequence, Variant 3 | 54 |
| Human RAD51 Protein Sequence, Variant 3 | 55 |
| Human MUS81 cDNA Sequence, Variant 1 | 56 |
| HUMAN MUS81 Protein Sequence, Variant 1 | 57 |
| Human MUS81 cDNA Sequence, Variant 2 | 58 |
| Human MUS81 Protein Sequence, Variant 2 | 59 |
| Human IFI16 cDNA Sequence, Variant 1 | 60 |
| HUMAN IFI16 Protein Sequence, Variant 1 | 61 |
| Human IFI16 cDNA Sequence, Variant 2 | 62 |
| Human IFI16 Protein Sequence, Variant 2 | 63 |
| Human IFI16 cDNA Sequence, Variant 3 | 64 |
| Human IFI16 Protein Sequence, Variant 3 | 65 |
| Human cGAS cDNA Sequence | 66 |
| Human cGAS Protein Sequence | 67 |
| Human DDX41 cDNA Sequence, Variant 1 | 68 |
| Human DDX41 Protein Sequence, Variant 1 | 69 |
| Human DDX41 cDNA Sequence, Variant 2 | 70 |
| HUMAN DDX41 Protein Sequence, Variant 2 | 71 |
| Human EXO1 cDNA Sequence, Variant 1 | 72 |
| Human EXO1 Protein Sequence, Variant 1 | 73 |
| Human EXO cDNA Sequence, Variant 2 | 74 |
| HUMAN EXO Protein Sequence, Variant 2 | 75 |
| Human EXO cDNA Sequence, Variant 3 | 76 |
| Human EXO Protein Sequence, Variant 3 | 77 |
| Human DNA2 cDNA Sequence | 78 |
| Human DNA2 Protein Sequence | 79 |
| Human RBBP8 cDNA Sequence, Variant 1 | 80 |
| Human RBBP8 Protein Sequence, Variant 1 | 81 |
| Human RBBP8 cDNA Sequence, Variant 2 | 82 |
| Human RBBP8 Protein Sequence, Variant 2 | 83 |
| Human MRE11 cDNA Sequence, Variant 1 | 84 |
| Human MRE11 Protein Sequence, Variant 1 | 85 |
| Human MRE11 cDNA Sequence, Variant 2 | 86 |
| Human MRE11 Protein Sequence, Variant 2 | 87 |
| Human MRE11 cDNA Sequence, Variant 3 | 88 |
| Human MRE11 Protein Sequence, Variant 3 | 89 |

Some embodiments of any of the methods described herein include determining the level of expression of a mRNA or a protein encoded by of one or more of STING, TREX1, BRCA1, BRCA2, SAMHD1, DNASE2, BLM, PARP1, RPA1, RAD51, MUS81, IFI16, cGAS, DDX41, EXO1, DNA2, RBBP8 (CtIP), and MRE11. In some examples of any of the methods described herein, increased STING or cGAS signaling activity can include, e.g., detecting a decreased level of a mRNA or a protein encoded by one or more of BRCA1, BRCA2, SAMHD1, DNASE2, BLM, PARP1, RPA1, and RAD51, and/or detecting an increased level of a mRNA or protein encoded by one or more of STING, MUS81, IFI16, cGAS, DDX41, EXO1, DNA2, RBBP8 (CtIP), and MRE11 in a mammalian cell (e.g., as compared to any of the exemplary reference levels described herein).

Some embodiments of any of the methods described herein, an increased cGAS/STING signaling activity can be determined by detecting of a gain-of-function mutation (e.g., a gene amplification or one or more activating amino acid substitutions in a protein encoded by one or more of MUS81, IFI16, cGAS, DDX41, EXO1, DNA2, RBBP8 (CtIP), and MRE1); a gene deletion of one or more of BRCA1, BRCA2, SAMHD1, DNASE2, BLM, PARP1, RPA1, and RAD51; one or more amino acid deletions in a protein encoded by one or more of BRCA1, BRCA2, SAMHD1, DNASE2, BLM, PARP1, RPA1, and RAD51; one or more inactivating amino acid mutations in a protein encoded by one or more of BRCA1, BRCA2, SAMHD1, DNASE2, BLM, PARP1, RPA1, or RAD51; or a frameshift mutation in one or more of BRCA1, BRCA2, SAMHD1, DNASE2, BLM, PARP1, RPA1, and RAD51.

In some embodiments of any of the methods described herein can include determining the level of expression of a mRNA or a protein encoded by TREX1. In some embodiments, a decreased level and/or activity of TREX1 can be determined by detection of a loss-of-function TREX1 mutation, a TREX1 gene deletion, one or more amino acid deletions in a protein encoded by a TREX1 gene, and one or more amino acid substitutions in a protein encoded by a TREX1 gene).

Methods of detecting a level of each of these exemplary cGAS/STING signaling pathway activities are described herein. Additional examples of cGAS/STING signaling pathway activities are known in the art, as well as methods for detecting a level of the same.

As used herein, “gain-of-function mutation” refers to one or more nucleotide substitutions, deletions, and/or insertions in a gene that results in the production of a protein encoded by the gene that has one or more increased activities in a mammalian cell as compared to the version of the protein encoded by the corresponding wildtype gene. In some embodiments, a gain-of-function mutation can be a gene amplification or one or more activating amino acid substitutions in a protein encoded by one or more of MUS81, IFI16, cGAS, DDX41, EXO1, DNA2, RBBP8 (CtIP), STING, and MRE1.

As used herein, “loss-of-function mutation” refers to one or more nucleotide substitutions, deletions, and/or insertions in gene that results in: a decrease in the level of expression of the encoded protein as compared to the level of the expression by the corresponding wildtype gene, and/or the expression of a protein encoded gene that has one or more decreased activities in a mammalian cell as compared to the version of the protein encoded by the corresponding wildtype gene. In some embodiments, a loss-of-function mutation can be a gene deletion, one or more amino acid deletions in a protein encoded by a gene, or one or more inactivating amino acid substitutions in a protein encoded by a gene.

The terms “hydrogen” and “H” are used interchangeably herein.

The term “halo” refers to fluoro (F), chloro (Cl), bromo (Br), or iodo (I).

The term “alkyl” refers to a hydrocarbon chain that may be a straight chain or branched chain, containing the indicated number of carbon atoms. For example, C1-10 indicates that the group may have from 1 to 10 (inclusive) carbon atoms in it. Non-limiting examples include methyl, ethyl, iso-propyl, tert-butyl, n-hexyl.

The term “haloalkyl” refers to an alkyl, in which one or more hydrogen atoms is/are replaced with an independently selected halo.

The term “alkoxy” refers to an —O-alkyl radical (e.g., —OCH3).

The term “carbocyclic ring” as used herein includes an aromatic or nonaromatic cyclic hydrocarbon group having 3 to 10 carbons, such as 3 to 8 carbons, such as 3 to 7 carbons, which may be optionally substituted. Examples of carbocyclic rings include five-membered, six membered, and seven-membered carbocyclic rings.

The term “heterocyclic ring” refers to an aromatic or nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclic rings include five-membered, six membered, and seven-membered heterocyclic rings.

The term “cycloalkyl” as used herein includes an aromatic or nonaromatic cyclic hydrocarbon radical having 3 to 10 carbons, such as 3 to 8 carbons, such as 3 to 7 carbons, wherein the cycloalkyl group which may be optionally substituted. Examples of cycloalkyls include five membered, six-membered, and seven-membered rings. Examples include cyclopropyl, cyclobutyl, cyclopentyl, cyclopentenyl, cyclohexyl, cyclohexenyl, cycloheptyl, and cyclooctyl.

The term “heterocycloalkyl” refers to an aromatic or nonaromatic 5-8 membered monocyclic, 8-12 membered bicyclic, or 11-14 membered tricyclic ring system radical having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocycloalkyls include five-membered, six-membered, and seven-membered heterocyclic rings. Examples include piperazinyl, pyrrolidinyl, dioxanyl, morpholinyl, tetrahydrofuranyl, and the like.

The term “hydroxy” refers to an OH group.

The term “amino” refers to an NH2 group.

The term “oxo” refers to O. By way of example, substitution of a CH2 a group with oxo gives a C═O group.

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

Other features and advantages of the invention will be apparent from the following detailed description and figures, and from the claims.

### DETAILED DESCRIPTION

The present invention is based on the discovery that cancer cells having decreased TREX1 level and/or activity and/or increased cGAS/STING signaling pathway activity are more sensitive to treatment with a STING antagonist or cGAS inhibitor. In view of these discoveries, provided herein are methods of treating a subject in need thereof with a treatment including a STING antagonist or cGAS inhibitor, methods of selecting a treatment for a subject in need thereof, where the treatment includes a STING antagonist or cGAS inhibitor, methods of selecting a subject for treatment with a STING antagonist or cGAS inhibitor, methods of selecting a subject for participation in a clinical trial with a STING antagonist or cGAS inhibitor, and methods of predicting a subject's responsiveness to a STING antagonist or cGAS inhibitor (e.g., a compound of any one of Formulas I-X or a compound shown in any one of Tables 1-10).

Non-liming aspects of these methods are described below, and can be used in any combination without limitation. Additional aspects of these methods are known in the art.

### Methods of Treating

Provided herein are methods of treating a subject (e.g., any of the exemplary subjects described herein) in need thereof that include: (a) identifying a subject having a cell (e.g., a cancer cell) having (i) decreased TREX1 level and/or activity (e.g., a decrease of 1% to about 99%, or any subranges of this range described herein) (e.g., as compared to a reference level), and/or (ii) an increased cGAS/STING signaling pathway activity (e.g., an increase of between 1% and 1000%, or any of the subranges of this range described herein) (e.g., as compared to a reference level); and (b) administering a treatment comprising a therapeutically effective amount of an STING antagonist or cGAS inhibitor (e.g., any of the exemplary STING antagonists or cGAS inhibitors described herein) or a pharmaceutically acceptable salt, solvate, or co-crystal thereof to the identified subject.

Also provided herein are methods of treating a subject (e.g., any of the exemplary subjects described herein) in need thereof that include: administering a treatment comprising a therapeutically effective amount of a STING antagonist or cGAS inhibitor (e.g., any of the exemplary STING antagonists or cGAS inhibitors described herein) or a pharmaceutically acceptable salt, solvate, or co-crystal thereof to a subject identified as having a cell (e.g., a cancer cell) having one or both of (i) decreased TREX1 level and/or activity (e.g., a decrease of about 1% to about 99%, or any subranges of this range described herein) (e.g., as compared to a reference level), and (ii) increased cGAS/STING signaling pathway activity (e.g., an increase of between 1% and 1000%, or any of the subranges of this range described herein) (e.g., as compared to a reference level) or to a subject identified as having an elevated level of cGAMP in serum or tumor (e.g., an increase of between 1% and 1000%, or any of the subranges of this range described herein) (e.g., as compared to a reference level).

In some embodiments, the subject is identified as having a cancer cell having decreased TREX1 level and/or activity. In some embodiments, the subject is identified as having an elevated level of cGAMP in a serum or tumor sample from the subject. In some embodiments, the subject is identified as having a cancer cell having increased cGAS/STING signaling pathway activity (e.g., an increase of between 1% and 1000%, or any of the subranges of this range described herein) (e.g., as compared to a reference level) or to a subject identified as having an elevated level of cGAMP in serum or tumor (e.g., an increase of between 1% and 1000%, or any of the subranges of this range described herein) (e.g., as compared to a reference level). In some embodiments, the subject is identified having a cancer cell having both (i) decreased TREX1 level and/or activity and (ii) increased cGAS/STING signaling pathway activity (e.g., an increase of between 1% and 1000%, or any of the subranges of this range described herein) (e.g., as compared to a reference level) or to a subject identified as having an elevated level of cGAMP in serum or tumor (e.g., an increase of between 1% and 1000%, or any of the subranges of this range described herein) (e.g., as compared to a reference level). In some embodiments, the subject is identified as having a cancer cell having decreased TREX1 level. In some embodiments, the TREX1 level is a level of TREX1 protein in the cancer cell. In some embodiments, the identification of the subject as having a cancer cell having a decreased TREX1 level includes detecting a decreased level of TREX1 protein in the cancer cell. In some embodiments, the TREX1 level is a level of TREX1 mRNA in the cancer cell. In some embodiments, the identification of the subject as having a cancer cell having a decreased TREX1 level comprises detecting a decreased level of TREX1 mRNA in the cancer cell.

In some embodiments, the decreased TREX1 level and/or activity is a result of TREX1 gene loss in the cancer cell. In some embodiments, the TREX1 gene loss is loss of one allele of the TREX1 gene. In some embodiments, the TREX1 gene loss is loss of both alleles of the TREX1 gene. In some embodiments, the identification of the subject as having a cancer cell having decreased TREX1 level and/or activity comprises detecting TREX1 gene loss in the cancer cell. In some embodiments, the decreased TREX1 level and/or activity is a result of one or more amino acid deletions or post-translational modifications of a protein encoded by a TREX1 gene in the cancer cell. In some embodiments, the identification of the subject as having a cancer cell having decreased TREX1 level and/or activity comprises detecting one or more amino acid deletions in a protein encoded by a TREX1 gene in the cancer cell. In some embodiments, the decreased TREX1 level and/or activity is a result of one or more inactivating amino acid substitutions in a protein encoded by a TREX1 gene in the cancer cell. In some embodiments, the identification of the subject as having a cancer cell having decreased TREX1 expression and/or activity comprises detecting one or more inactivating amino acid substitutions or post-translational modifications in a protein encoded by a TREX1 gene in the cancer cell.

In some embodiments, the increased cGAS/STING signaling pathway activity is a result of a decreased level and/or activity of BRCA1 in the cancer cell. In some embodiments, the decreased level and/or activity of BRCA1 in the cancer cell is a result of a frameshift mutation in a BRCA1 gene. In some embodiments, frameshift mutation in a BRCA1 gene is a E111Gfs*3 frameshift insertion (e.g., a mutation in a BRCA1 gene that causes a E111Gfs*3 frameshift insertion with respect to SEQ ID NO: 15). In some embodiments, the decreased level and/or activity of BRCA1 in the cancer cell is a result of BRCA1 gene loss in the cancer cell. In some embodiments, the decreased level and/or activity of BRCA1 in the cancer cell is a result of one or more amino acid deletions in a protein encoded by a BRCA1 gene. In some embodiments, the decreased level and/or activity of BRCA1 in the cancer cell is a result of one or more inactivating amino acid substitutions in a protein encoded by a BRCA1 gene.

In some embodiments, the increased cGAS/STING signaling pathway activity is a result of a decreased level and/or activity of BRCA2 gene. In some embodiments, the decreased level and/or activity of BRCA2 in the cancer cell is a result of a frameshift mutation in a BRCA2 gene. In some embodiments, the frameshift mutation in a BRCA2 gene is a N1784Kfs*3 frameshift insertion (e.g., a mutation in a BRCA2 gene that causes a N1784Kfs*3 frameshift insertion with respect to SEQ ID NO: 25). In some embodiments, the decreased level and/or activity of BRCA2 in the cancer cell is a result of BRCA2 gene loss in the cancer cell. In some embodiments, the decreased level and/or activity of BRCA2 in the cancer cell is a result of one or more amino acid deletions in a protein encoded by a BRCA2 gene. In some embodiments, decreased level and/or activity of BRCA2 in the cancer cell is a result of one or more inactivating amino acid substitutions in a protein encoded by a BRCA2 gene.

In some embodiments, the increased cGAS/STING signaling pathway activity is a result of a decreased level and/or activity of SAMHD1 in the cancer cell. In some embodiments, the decreased level and/or activity of SAMHD1 in the cancer cell is a result of one or more inactivating amino acid substitutions in a protein encoded by a SAMHD1 gene in the cancer cell. In some embodiments, the one or more inactivating amino acid substitutions in a protein encoded by a SAMHD1 gene is a V133I amino acid substitution (e.g., a mutation in a SAMHD1 gene that causes a V133I amino acid substitution with respect to SEQ ID NO: 27). In some embodiments, the decreased level and/or activity of SAMHD1 in the cancer cell is a result of SAMHD1 gene loss in the cancer cell. In some embodiments, the decreased level and/or activity of SAMHD1 in the cancer cell is a result of one or more amino acid deletions in a protein encoded by a SAMHD1 gene.

In some embodiments, the increased cGAS/STING signaling pathway activity is a result of a decreased level and/or activity of DNASE2 in the cancer cell. In some embodiments, the decreased level and/or activity of DNASE2 in the cancer cell is a result of one or more inactivating amino acid substitutions in a protein encoded by a DNASE2 gene in the cancer cell. In some embodiments, the one or more inactivating amino acid substitutions in a protein encoded by a DNASE2 gene is a R314W amino acid substitution (e.g., a mutation in a DNASE2 gene that causes a R314W amino acid substitution with respect to SEQ ID NO: 33). In some embodiments, the decreased level and/or activity of DNASE2 in the cancer cell is a result of DNASE2 gene loss in the cancer cell. In some embodiments, the decreased level and/or activity of DNASE2 in the cancer cell is a result of one or more amino acid deletions in a protein encoded by a DNASE2 gene.

In some embodiments, the increased cGAS/STING signaling pathway activity is a result of a decreased level and/or activity of BLM in the cancer cell. In some embodiments, the decreased level and/or activity of BLM in the cancer cell is a result of a frameshift mutation in a BLM gene. In some embodiments, the frameshift mutation in a BLM gene is a N515Mfs*16 frameshift deletion (e.g., a mutation in a BLM gene that causes a N515Mfs*16 frameshift deletion with respect to SEQ ID NO: 37). In some embodiments, the decreased level and/or activity of BLM in the cancer cell is a result of BLM gene loss in the cancer cell. In some embodiments, the decreased level and/or activity of BLM in the cancer cell is a result of one or more amino acid deletions in a protein encoded by a BLM gene. In some embodiments, the decreased level and/or activity of BLM in the cancer cell is a result of one or more inactivating amino acid substitutions in a protein encoded by a BLM gene.

In some embodiments, the increased cGAS/STING signaling pathway activity is a result of a decreased level and/or activity of PARP1 in the cancer cell. In some embodiments, the decreased level and/or activity of PARP1 in the cancer cell is a result of a frameshift mutation in a PARP1 gene. In some embodiments, the frameshift mutation in a PARP1 gene is a S507Afs*17 frameshift deletion (e.g., a mutation in a PARP1 gene that causes a S507Afs*17 frameshift deletion with respect to SEQ ID NO: 43). In some embodiments, the decreased level and/or activity of PARP1 in the cancer cell is a result of PARP1 gene loss in the cancer cell. In some embodiments, the decreased level and/or activity of PARP1 in the cancer cell is a result of one or more amino acid deletions in a protein encoded by a PARP1 gene. In some embodiments, the decreased level and/or activity of PARP1 in the cancer cell is a result of one or more inactivating amino acid substitutions in a protein encoded by a PARP1 gene.

In some embodiments, the increased cGAS/STING signaling pathway activity is a result of a decreased level and/or activity of RPA1 in the cancer cell. In some embodiments, the decreased level and/or activity of RPA1 in the cancer cell is a result of a mutation that results in aberrant RPA1 mRNA splicing in the cancer cell. In some embodiments, the mutation that results in aberrant RPA1 mRNA splicing in the cancer cell is a X12 splice mutation. In some embodiments, the decreased level and/or activity of RPA1 in the cancer cell is a result of RPA1 gene loss in the cancer cell. In some embodiments, the decreased level and/or activity of RPA1 in the cancer cell is a result of one or more amino acid deletions in a protein encoded by a RPA1 gene. In some embodiments, the decreased level and/or activity of RPA1 in the cancer cell is a result of one or more inactivating amino acid substitutions in a protein encoded by a RPA1 gene.

In some embodiments, the increased cGAS/STING signaling pathway activity is a result of a decreased level and/or activity of RAD51 in the cancer cell. In some embodiments, the decreased level and/or activity of RAD51 in the cancer cell is a result of one or more inactivating amino acid substitutions in a protein encoded by a RAD51 gene. In some embodiments, the one or more inactivating amino acid substitutions in a protein encoded by a RAD51 gene is an R254* amino acid substitution (e.g., a mutation in a RAD51 gene that causes a R254* amino acid substitution with respect to SEQ ID NO: 51). In some embodiments, the decreased level and/or activity of RAD51 in the cancer cell is a result of RAD51 gene loss in the cancer cell. In some embodiments, the decreased level and/or activity of RAD51 in the cancer cell is a result of one or more amino acid deletions in a protein encoded by a RAD51 gene.

In some embodiments, the increased cGAS/STING signaling pathway activity is a result of an increased level and/or activity of MUS81 in the cancer cell. In some embodiments, the increased level and/or activity of MUS81 in the cancer cell is a result of MUS81 gene amplification in the cancer cell. In some embodiments, increased level and/or activity of MUS81 in the cancer cell is a result of one or more activating amino acid substitutions in a protein encoded by a MUS81 gene.

In some embodiments, increased cGAS/STING signaling pathway activity is a result of an increased level and/or activity of IFI16 in the cancer cell. In some embodiments, the increased level and/or activity of IFI16 in the cancer cell is a result of IFI16 gene amplification in the cancer cell. In some embodiments, increased level and/or activity of IFI16 in the cancer cell is a result of one or more activating amino acid substitutions in a protein encoded by an IFI16 gene.

In some embodiments, the increased cGAS/STING signaling pathway activity is a result of an increased level and/or activity of cGAS in the cancer cell. In some embodiments, the increased level and/or activity of cGAS in the cancer cell is a result of cGAS gene amplification in the cancer cell. In some embodiments, the increased level and/or activity of cGAS in the cancer cell is a result of one or more activating amino acid substitutions in a protein encoded by a cGAS gene.

In some embodiments, the increased cGAS/STING signaling pathway activity is a result of an increased activity of STING in the cancer cell. In some embodiments, the increased activity of STING in the cancer cell is a result of one or more activating amino acid substitutions in a protein encoded by a STING gene.

In some embodiments, the increased cGAS/STING signaling pathway activity is a result of an increased level and/or activity of DDX41 in the cancer cell. In some embodiments, the increased level and/or activity of DDX41 in the cancer cell is a result of DDX41 gene amplification in the cancer cell. In some embodiments, the increased level and/or activity of DDX41 in the cancer cell is a result of one or more activating amino acid substitutions in a protein encoded by a DDX41 gene.

In some embodiments, the increased cGAS/STING signaling pathway activity is a result of an increased level and/or activity of EXO1 in the cancer cell. In some embodiments, the increased level and/or activity of EXO1 in the cancer cell is a result of EXO1 gene amplification in the cancer cell. In some embodiments, increased level and/or activity of EXO1 in the cancer cell is a result of one or more activating amino acid substitutions in a protein encoded by a EXO1 gene.

In some embodiments, the increased cGAS/STING signaling pathway activity is a result of an increased level and/or activity of DNA2 in the cancer cell. In some embodiments, the increased level and/or activity of DNA2 in the cancer cell is a result of DNA2 gene amplification in the cancer cell. In some embodiments, the increased level and/or activity of DNA2 in the cancer cell is a result of one or more activating amino acid substitutions in a protein encoded by a DNA2 gene.

In some embodiments, the increased cGAS/STING signaling pathway activity is a result of an increased level and/or activity of RBBP8 (CtIP) in the cancer cell. In some embodiments, the increased level and/or activity of RBBP8 (CtIP) in the cancer cell is a result of RBBP8 (CtIP) gene amplification in the cancer cell. In some embodiments, the increased level and/or activity of RBBP8 (CtIP) in the cancer cell is a result of one or more activating amino acid substitutions in a protein encoded by a RBBP8 (CtIP) gene.

In some embodiments, the increased cGAS/STING signaling pathway activity is a result of an increased level and/or activity of MRE11 in the cancer cell. In some embodiments, the increased level and/or activity of MRE11 in the cancer cell is a result of MRE11 gene amplification in the cancer cell. In some embodiments, the increased level and/or activity of MRE11 in the cancer cell is a result of one or more activating amino acid substitutions in a protein encoded by a MRE11 gene.

In some embodiments, the subject has been diagnosed or identified as having a cancer. In some embodiments, the cancer is selected from the group consisting of: renal clear cell carcinoma, uveal melanoma, tongue squamous cell carcinoma, breast cancer, and skin cancer.

In some embodiments of any of the methods described herein, the STING antagonist or cGAS inhibitor is an inhibitory nucleic acid (e.g., a short interfering RNA, an antisense nucleic acid, a cyclic dinucleotide, or a ribozyme). In some embodiments of any of the methods described herein, the STING antagonist or cGAS inhibitor is any of the compounds described herein, or a pharmaceutically acceptable salt, solvate, or co-crystal thereof, with the proviso that in embodiments related to a gain of function mutation in STING, a cGAS inhibitor is not employed in a method described herein.

In some embodiments of any of the methods of treatment described herein, the method can result in a decreased risk (e.g., a 1% to a 99% decrease, or any of the subranges of this range described herein) of developing a comorbidity in the subject (e.g., as compared to the risk of developing a comorbidity in a subject having cancer cells having a similar decreased TREX1 level and/or activity and/or increased cGAS/STING signaling pathway activity, but administered a different treatment or a placebo).

Additional exemplary aspects that can be used or incorporated in these methods are described herein.

### Methods of Selecting a Treatment for a Subject

Provided herein are methods of selecting a treatment for a subject (e.g., any of the exemplary subjects described herein) in need thereof that include: (a) identifying a subject having a cell (e.g., a cancer cell) having one or both of (i) decreased TREX1 level and/or activity (e.g., a decrease of about 1% to about 99%, or any subranges of this range described herein) (e.g., as compared to a reference level), and (ii) increased cGAS/STING signaling pathway activity (e.g., an increase of between 1% and 1000%, or any of the subranges of this range described herein) (e.g., as compared to a reference level)) and/or identifying a subject identified as having an elevated level of cGAMP in serum or tumor (e.g., an increase of between 1% and 1000%, or any of the subranges of this range described herein) (e.g., as compared to a reference level); and (b) selecting for the identified subject a treatment comprising a therapeutically effective amount of a STING antagonist or cGAS inhibitor (e.g., any of the exemplary STING antagonists or cGAS inhibitor described herein) or a pharmaceutically acceptable salt, solvate, or co-crystal thereof.

Provided herein are methods of selecting a treatment for a subject (e.g., any of the exemplary subjects described herein) in need thereof that include: selecting a treatment comprising a therapeutically effective amount of a STING antagonist or cGAS inhibitor (e.g., any of the exemplary STING antagonists or cGAS inhibitors described herein) or a pharmaceutically acceptable salt, solvate, or co-crystal thereof for a subject identified as having a cell (e.g., a cancer cell) having one or both of (i) decreased TREX1 level and/or activity (e.g., a decrease of about 1% to about 99%, or any subranges of this range described herein) (e.g., as compared to a reference level), (ii) increased cGAS/STING signaling pathway activity (e.g., an increase of between 1% and 1000%, or any of the subranges of this range described herein) (e.g., as compared to a reference level) or to a subject identified as having an elevated level of cGAMP in a serum or tumor sample (e.g., an increase of between 1% and 1000%, or any of the subranges of this range described herein) (e.g., as compared to a reference level). In some embodiments, the subject is identified as having a cancer cell having decreased TREX1 level and/or activity. In some embodiments, the subject is identified as having a cancer cell having increased cGAS/STING signaling pathway activity. In some embodiments, the subject is identified having a cancer cell having both (i) decreased TREX1 level and/or activity and (ii) increased cGAS/STING signaling pathway activity (e.g., an increase of between 1% and 1000%, or any of the subranges of this range described herein) (e.g., as compared to a reference level) or to a subject identified as having an elevated levels of cGAMP in a serum or tumor sample from the patient (e.g., an increase of between 1% and 1000%, or any of the subranges of this range described herein) (e.g., as compared to a reference level). In some embodiments, the subject is identified as having a cancer cell having decreased TREX1 level. In some embodiments, the TREX1 level is a level of TREX1 protein in the cancer cell. In some embodiments, the identification of the subject as having a cancer cell having a decreased TREX1 level includes detecting a decreased level of TREX1 protein in the cancer cell. In some embodiments, the TREX1 level is a level of TREX1 mRNA in the cancer cell. In some embodiments, the identification of the subject as having a cancer cell having a decreased TREX1 level comprises detecting a decreased level of TREX1 mRNA in the cancer cell.

In some embodiments, the decreased TREX1 level and/or activity is a result of TREX1 gene loss in the cancer cell. In some embodiments, the TREX1 gene loss is loss of one allele of the TREX1 gene. In some embodiments, the TREX1 gene loss is loss of both alleles of the TREX1 gene. In some embodiments, the identification of the subject as having a cancer cell having decreased TREX1 level and/or activity comprises detecting TREX1 gene loss in the cancer cell. In some embodiments, the decreased TREX1 level and/or activity is a result of one or more amino acid deletions in a protein encoded by a TREX1 gene in the cancer cell. In some embodiments, the identification of the subject as having a cancer cell having decreased TREX1 level and/or activity comprises detecting one or more amino acid deletions in a protein encoded by a TREX1 gene in the cancer cell. In some embodiments, the decreased TREX1 level and/or activity is a result of one or more inactivating amino acid substitutions in a protein encoded by a TREX1 gene in the cancer cell. In some embodiments, the identification of the subject as having a cancer cell having decreased TREX1 expression and/or activity comprises detecting one or more inactivating amino acid substitutions in a protein encoded by a TREX1 gene in the cancer cell.

In some embodiments, the increased cGAS/STING signaling pathway activity is a result of a decreased level and/or activity of BRCA1 in the cancer cell. In some embodiments, the decreased level and/or activity of BRCA1 in the cancer cell is a result of a frameshift mutation in a BRCA1 gene. In some embodiments, frameshift mutation in a BRCA1 gene is a E111Gfs*3 frameshift insertion (e.g., a mutation in a BRCA1 gene that causes a E111Gfs*3 frameshift insertion with respect to SEQ ID NO: 15). In some embodiments, the decreased level and/or activity of BRCA1 in the cancer cell is a result of BRCA1 gene loss in the cancer cell. In some embodiments, the decreased level and/or activity of BRCA1 in the cancer cell is a result of one or more amino acid deletions in a protein encoded by a BRCA1 gene. In some embodiments, the decreased level and/or activity of BRCA1 in the cancer cell is a result of one or more inactivating amino acid substitutions in a protein encoded by a BRCA1 gene.

In some embodiments, the increased cGAS/STING signaling pathway activity is a result of a decreased level and/or activity of BRCA2 gene. In some embodiments, the decreased level and/or activity of BRCA2 in the cancer cell is a result of a frameshift mutation in a BRCA2 gene. In some embodiments, the frameshift mutation in a BRCA2 gene is a N1784Kfs*3 frameshift insertion (e.g., a mutation in a BRCA2 gene that causes a N1784Kfs*3 frameshift insertion with respect to SEQ ID NO: 25). In some embodiments, the decreased level and/or activity of BRCA2 in the cancer cell is a result of BRCA2 gene loss in the cancer cell. In some embodiments, the decreased level and/or activity of BRCA2 in the cancer cell is a result of one or more amino acid deletions in a protein encoded by a BRCA2 gene. In some embodiments, decreased level and/or activity of BRCA2 in the cancer cell is a result of one or more inactivating amino acid substitutions in a protein encoded by a BRCA2 gene.

In some embodiments, the increased cGAS/STING signaling pathway activity is a result of a decreased level and/or activity of SAMHD1 in the cancer cell. In some embodiments, the decreased level and/or activity of SAMHD1 in the cancer cell is a result of one or more inactivating amino acid substitutions in a protein encoded by a SAMHD1 gene in the cancer cell. In some embodiments, the one or more inactivating amino acid substitutions in a protein encoded by a SAMHD1 gene is a V133I amino acid substitution (e.g., a mutation in a SAMHD1 gene that causes a V133I amino acid substitution with respect to SEQ ID NO: 27). In some embodiments, the decreased level and/or activity of SAMHD1 in the cancer cell is a result of SAMHD1 gene loss in the cancer cell. In some embodiments, the decreased level and/or activity of SAMHD1 in the cancer cell is a result of one or more amino acid deletions in a protein encoded by a SAMHD1 gene.

In some embodiments, the increased cGAS/STING signaling pathway activity is a result of a decreased level and/or activity of DNASE2 in the cancer cell. In some embodiments, the decreased level and/or activity of DNASE2 in the cancer cell is a result of one or more inactivating amino acid substitutions in a protein encoded by a DNASE2 gene in the cancer cell. In some embodiments, the one or more inactivating amino acid substitutions in a protein encoded by a DNASE2 gene is a R314W amino acid substitution (e.g., a mutation in a DNASE2 gene that causes a R314W amino acid substitution with respect to SEQ ID NO: 33). In some embodiments, the decreased level and/or activity of DNASE2 in the cancer cell is a result of DNASE2 gene loss in the cancer cell. In some embodiments, the decreased level and/or activity of DNASE2 in the cancer cell is a result of one or more amino acid deletions in a protein encoded by a DNASE2 gene.

In some embodiments, the increased cGAS/STING signaling pathway activity is a result of a decreased level and/or activity of BLM in the cancer cell. In some embodiments, the decreased level and/or activity of BLM in the cancer cell is a result of a frameshift mutation in a BLM gene. In some embodiments, the frameshift mutation in a BLM gene is a N515Mfs*16 frameshift deletion (e.g., a mutation in a BLM gene that causes a N515Mfs*16 frameshift deletion with respect to SEQ ID NO: 37). In some embodiments, the decreased level and/or activity of BLM in the cancer cell is a result of BLM gene loss in the cancer cell. In some embodiments, the decreased level and/or activity of BLM in the cancer cell is a result of one or more amino acid deletions in a protein encoded by a BLM gene. In some embodiments, the decreased level and/or activity of BLM in the cancer cell is a result of one or more inactivating amino acid substitutions in a protein encoded by a BLM gene.

In some embodiments, the increased cGAS/STING signaling pathway activity is a result of a decreased level and/or activity of PARP1 in the cancer cell. In some embodiments, the decreased level and/or activity of PARP1 in the cancer cell is a result of a frameshift mutation in a PARP1 gene. In some embodiments, the frameshift mutation in a PARP1 gene is a S507Afs*17 frameshift deletion (e.g., a mutation in a PARP1 gene that causes a S507Afs*17 frameshift deletion with respect to SEQ ID NO: 43). In some embodiments, the decreased level and/or activity of PARP1 in the cancer cell is a result of PARP1 gene loss in the cancer cell. In some embodiments, the decreased level and/or activity of PARP1 in the cancer cell is a result of one or more amino acid deletions in a protein encoded by a PARP1 gene. In some embodiments, the decreased level and/or activity of PARP1 in the cancer cell is a result of one or more inactivating amino acid substitutions in a protein encoded by a PARP1 gene.

In some embodiments, the increased cGAS/STING signaling pathway activity is a result of a decreased level and/or activity of RPA1 in the cancer cell. In some embodiments, the decreased level and/or activity of RPA1 in the cancer cell is a result of a mutation that results in aberrant RPA1 mRNA splicing in the cancer cell. In some embodiments, the mutation that results in aberrant RPA1 mRNA splicing in the cancer cell is a X12 splice mutation. In some embodiments, the decreased level and/or activity of RPA1 in the cancer cell is a result of RPA1 gene loss in the cancer cell. In some embodiments, the decreased level and/or activity of RPA1 in the cancer cell is a result of one or more amino acid deletions in a protein encoded by a RPA1 gene. In some embodiments, the decreased level and/or activity of RPA1 in the cancer cell is a result of one or more inactivating amino acid substitutions in a protein encoded by a RPA1 gene.

In some embodiments, the increased cGAS/STING signaling pathway activity is a result of a decreased level and/or activity of RAD51 in the cancer cell. In some embodiments, the decreased level and/or activity of RAD51 in the cancer cell is a result of one or more inactivating amino acid substitutions in a protein encoded by a RAD51 gene. In some embodiments, the one or more inactivating amino acid substitutions in a protein encoded by a RAD51 gene is an R254* amino acid substitution (e.g., a mutation in a RAD51 gene that causes a R254* amino acid substitution with respect to SEQ ID NO: 51). In some embodiments, the decreased level and/or activity of RAD51 in the cancer cell is a result of RAD51 gene loss in the cancer cell. In some embodiments, the decreased level and/or activity of RAD51 in the cancer cell is a result of one or more amino acid deletions in a protein encoded by a RAD51 gene.

In some embodiments, the increased cGAS/STING signaling pathway activity is a result of an increased level and/or activity of MUS81 in the cancer cell. In some embodiments, the increased level and/or activity of MUS81 in the cancer cell is a result of MUS81 gene amplification in the cancer cell. In some embodiments, increased level and/or activity of MUS81 in the cancer cell is a result of one or more activating amino acid substitutions in a protein encoded by a MUS81 gene.

In some embodiments, increased cGAS/STING signaling pathway activity is a result of an increased level and/or activity of IFI16 in the cancer cell. In some embodiments, the increased level and/or activity of IFI16 in the cancer cell is a result of IFI16 gene amplification in the cancer cell. In some embodiments, increased level and/or activity of IFI16 in the cancer cell is a result of one or more activating amino acid substitutions in a protein encoded by an IFI16 gene.

In some embodiments, the increased cGAS/STING signaling pathway activity is a result of an increased level and/or activity of cGAS in the cancer cell. In some embodiments, the increased level and/or activity of cGAS in the cancer cell is a result of cGAS gene amplification in the cancer cell. In some embodiments, the increased level and/or activity of cGAS in the cancer cell is a result of one or more activating amino acid substitutions in a protein encoded by a cGAS gene.

In some embodiments, the increased cGAS/STING signaling pathway activity is a result of an increased activity of STING in the cancer cell. In some embodiments, the increased activity of STING in the cancer cell is a result of one or more activating amino acid substitutions in a protein encoded by a STING gene.

In some embodiments, the increased cGAS/STING signaling pathway activity is a result of an increased level and/or activity of DDX41 in the cancer cell. In some embodiments, the increased level and/or activity of DDX41 in the cancer cell is a result of DDX41 gene amplification in the cancer cell. In some embodiments, the increased level and/or activity of DDX41 in the cancer cell is a result of one or more activating amino acid substitutions in a protein encoded by a DDX41 gene.

In some embodiments, the increased cGAS/STING signaling pathway activity is a result of an increased level and/or activity of EXO1 in the cancer cell. In some embodiments, the increased level and/or activity of EXO1 in the cancer cell is a result of EXO1 gene amplification in the cancer cell. In some embodiments, increased level and/or activity of EXO1 in the cancer cell is a result of one or more activating amino acid substitutions in a protein encoded by a EXO1 gene.

In some embodiments, the increased cGAS/STING signaling pathway activity is a result of an increased level and/or activity of DNA2 in the cancer cell. In some embodiments, the increased level and/or activity of DNA2 in the cancer cell is a result of DNA2 gene amplification in the cancer cell. In some embodiments, the increased level and/or activity of DNA2 in the cancer cell is a result of one or more activating amino acid substitutions in a protein encoded by a DNA2 gene.

In some embodiments, the increased cGAS/STING signaling pathway activity is a result of an increased level and/or activity of RBBP8 (CtIP) in the cancer cell. In some embodiments, the increased level and/or activity of RBBP8 (CtIP) in the cancer cell is a result of RBBP8 (CtIP) gene amplification in the cancer cell. In some embodiments, the increased level and/or activity of RBBP8 (CtIP) in the cancer cell is a result of one or more activating amino acid substitutions in a protein encoded by a RBBP8 (CtIP) gene.

In some embodiments, the increased cGAS/STING signaling pathway activity is a result of an increased level and/or activity of MRE11 in the cancer cell. In some embodiments, the increased level and/or activity of MRE11 in the cancer cell is a result of MRE11 gene amplification in the cancer cell. In some embodiments, the increased level and/or activity of MRE11 in the cancer cell is a result of one or more activating amino acid substitutions in a protein encoded by a MRE11 gene.

In some embodiments, the subject has been diagnosed or identified as having a cancer. In some embodiments, the cancer is selected from the group consisting of: renal clear cell carcinoma, uveal melanoma, tongue squamous cell carcinoma, breast cancer, and skin cancer. In some embodiments, the methods further comprise administering the selected treatment to the subject.

In some embodiments of any of the methods described herein, the STING antagonist or cGAS inhibitor is an inhibitory nucleic acid (e.g., a short interfering RNA, an antisense nucleic acid, a cyclic dinucleotide, or a ribozyme). In some embodiments of any of the methods described herein, the STING antagonist or cGAS inhibitor is any of the STING antagonists or cGAS inhibitors described herein, or a pharmaceutically acceptable salt, solvate, or co-crystal thereof. In some embodiments including a gain of function mutation in STING, a cGAS inhibitor is not employed in a method of the present disclosure.

Some embodiments of any of the methods described herein can further include recording the selected treatment in the subject's clinical record (e.g., a computer readable medium). Some embodiments of any of the methods described herein can further include administering one or more doses (e.g., at least two, at least four, at least six, at least eight, at least ten doses) of the selected treatment to the identified subject.

Additional exemplary aspects that can be used or incorporated in these methods are described herein.

### Methods of Selecting a Subject for Treatment

Also provided herein are methods of selecting a subject for treatment that include: (a) identifying a subject (e.g., any of the subjects described herein) having a cell (e.g., a cancer cell) having one or both of (i) decreased TREX1 level and/or activity (e.g., a decrease of about 1% to about 99%, or any subranges of this range described herein) (e.g., as compared to a reference level), and (ii) increased cGAS/STING signaling pathway activity (e.g., an increase of between 1% and 1000%, or any of the subranges of this range described herein) (e.g., as compared to a reference level); and/or identifying a subject as having an elevated level of cGAMP in a serum or a tumor sample (e.g., an increase of between 1% and 1000%, or any of the subranges of this range described herein) (e.g., as compared to a reference level); and (b) selecting an identified subject for treatment with a therapeutically effective amount of a STING antagonist or cGAS inhibitor (e.g., any of the exemplary STING antagonists or cGAS inhibitors described herein or known in the art) or a pharmaceutically acceptable salt, solvate, or co-crystal thereof.

Also provided herein are methods of selecting a subject for treatment that include selecting a subject (e.g., any of the subjects described herein) identified as having a cell (e.g., a cancer cell) having one or both of (i) decreased TREX1 level and/or activity (e.g., a decrease to about 1% to about 99%, or any subranges of this range described herein) (e.g., as compared to a reference level), and (ii) increased cGAS/STING signaling pathway activity (e.g., an increase of between 1% and 1000%, or any of the subranges of this range described herein) (e.g., as compared to a reference level), and/or selecting a subject identified as having ab elevated level of cGAMP in a serum or a tumor sample (e.g., an increase of between 1% and 1000%, or any of the subranges of this range described herein) (e.g., as compared to a reference level), for treatment with a therapeutically effective amount of a STING antagonist or cGAS inhibitor (e.g., any of the exemplary STING antagonists or cGAS inhibitor described herein or known in the art) or a pharmaceutically acceptable salt, solvate, or co-crystal thereof.

In some embodiments, the subject is identified as having a cancer cell having decreased TREX1 level and/or activity. In some embodiments, the subject is identified as having an elevated level of cGAMP in a serum or a tumor sample as compared to a reference sample. In some embodiments, the subject is identified as having a cancer cell having increased cGAS/STING signaling pathway activity. In some embodiments, the subject is identified having a cancer cell having both (i) decreased TREX1 level and/or activity and (ii) increased cGAS/STING signaling pathway activity (e.g., an increase of between 1% and 1000%, or any of the subranges of this range described herein) (e.g., as compared to a reference level) or to a subject identified as having an elevated level of cGAMP in a serum or a tumor sample (e.g., an increase of between 1% and 1000%, or any of the subranges of this range described herein) (e.g., as compared to a reference level). In some embodiments, the subject is identified as having a cancer cell having decreased TREX1 level. In some embodiments, the TREX1 level is a level of TREX1 protein in the cancer cell. In some embodiments, the identification of the subject as having a cancer cell having a decreased TREX1 level includes detecting a decreased level of TREX1 protein in the cancer cell. In some embodiments, the TREX1 level is a level of TREX1 mRNA in the cancer cell. In some embodiments, the identification of the subject as having a cancer cell having a decreased TREX1 level comprises detecting a decreased level of TREX1 mRNA in the cancer cell.

In some embodiments, the decreased TREX1 level and/or activity is a result of TREX1 gene loss in the cancer cell. In some embodiments, the TREX1 gene loss is loss of one allele of the TREX1 gene. In some embodiments, the TREX1 gene loss is loss of both alleles of the TREX1 gene. In some embodiments, the identification of the subject as having a cancer cell having decreased TREX1 level and/or activity comprises detecting TREX1 gene loss in the cancer cell. In some embodiments, the decreased TREX1 level and/or activity is a result of one or more amino acid deletions in a protein encoded by a TREX1 gene in the cancer cell. In some embodiments, the identification of the subject as having a cancer cell having decreased TREX1 level and/or activity comprises detecting one or more amino acid deletions in a protein encoded by a TREX1 gene in the cancer cell. In some embodiments, the decreased TREX1 level and/or activity is a result of one or more inactivating amino acid substitutions in a protein encoded by a TREX1 gene in the cancer cell. In some embodiments, the identification of the subject as having a cancer cell having decreased TREX1 expression and/or activity comprises detecting one or more inactivating amino acid substitutions in a protein encoded by a TREX1 gene in the cancer cell.

In some embodiments, the increased cGAS/STING signaling pathway activity is a result of a decreased level and/or activity of BRCA1 in the cancer cell. In some embodiments, the decreased level and/or activity of BRCA1 in the cancer cell is a result of a frameshift mutation in a BRCA1 gene. In some embodiments, frameshift mutation in a BRCA1 gene is a E111Gfs*3 frameshift insertion (e.g., a mutation in a BRCA1 gene that causes a E111Gfs*3 frameshift insertion with respect to SEQ ID NO: 15). In some embodiments, the decreased level and/or activity of BRCA1 in the cancer cell is a result of BRCA1 gene loss in the cancer cell. In some embodiments, the decreased level and/or activity of BRCA1 in the cancer cell is a result of one or more amino acid deletions in a protein encoded by a BRCA1 gene. In some embodiments, the decreased level and/or activity of BRCA1 in the cancer cell is a result of one or more inactivating amino acid substitutions in a protein encoded by a BRCA1 gene.

In some embodiments, the increased cGAS/STING signaling pathway activity is a result of a decreased level and/or activity of BRCA2 gene. In some embodiments, the decreased level and/or activity of BRCA2 in the cancer cell is a result of a frameshift mutation in a BRCA2 gene. In some embodiments, the frameshift mutation in a BRCA2 gene is a N1784Kfs*3 frameshift insertion (e.g., a mutation in a BRCA2 gene that causes a N1784Kfs*3 frameshift insertion with respect to SEQ ID NO: 25). In some embodiments, the decreased level and/or activity of BRCA2 in the cancer cell is a result of BRCA2 gene loss in the cancer cell. In some embodiments, the decreased level and/or activity of BRCA2 in the cancer cell is a result of one or more amino acid deletions in a protein encoded by a BRCA2 gene. In some embodiments, decreased level and/or activity of BRCA2 in the cancer cell is a result of one or more inactivating amino acid substitutions in a protein encoded by a BRCA2 gene.

In some embodiments, the increased cGAS/STING signaling pathway activity is a result of a decreased level and/or activity of SAMHD1 in the cancer cell. In some embodiments, the decreased level and/or activity of SAMHD1 in the cancer cell is a result of one or more inactivating amino acid substitutions in a protein encoded by a SAMHD1 gene in the cancer cell. In some embodiments, the one or more inactivating amino acid substitutions in a protein encoded by a SAMHD1 gene is a V133I amino acid substitution (e.g., a mutation in a SAMHD1 gene that causes a V133I amino acid substitution with respect to SEQ ID NO: 27). In some embodiments, the decreased level and/or activity of SAMHD1 in the cancer cell is a result of SAMHD1 gene loss in the cancer cell. In some embodiments, the decreased level and/or activity of SAMHD1 in the cancer cell is a result of one or more amino acid deletions in a protein encoded by a SAMHD1 gene.

In some embodiments, the increased cGAS/STING signaling pathway activity is a result of a decreased level and/or activity of DNASE2 in the cancer cell. In some embodiments, the decreased level and/or activity of DNASE2 in the cancer cell is a result of one or more inactivating amino acid substitutions in a protein encoded by a DNASE2 gene in the cancer cell. In some embodiments, the one or more inactivating amino acid substitutions in a protein encoded by a DNASE2 gene is a R314W amino acid substitution (e.g., a mutation in a DNASE2 gene that causes a R314W amino acid substitution with respect to SEQ ID NO: 33). In some embodiments, the decreased level and/or activity of DNASE2 in the cancer cell is a result of DNASE2 gene loss in the cancer cell. In some embodiments, the decreased level and/or activity of DNASE2 in the cancer cell is a result of one or more amino acid deletions in a protein encoded by a DNASE2 gene.

In some embodiments, the increased cGAS/STING signaling pathway activity is a result of a decreased level and/or activity of BLM in the cancer cell. In some embodiments, the decreased level and/or activity of BLM in the cancer cell is a result of a frameshift mutation in a BLM gene. In some embodiments, the frameshift mutation in a BLM gene is a N515Mfs*16 frameshift deletion (e.g., a mutation in a BLM gene that causes a N515Mfs*16 frameshift deletion with respect to SEQ ID NO: 37). In some embodiments, the decreased level and/or activity of BLM in the cancer cell is a result of BLM gene loss in the cancer cell. In some embodiments, the decreased level and/or activity of BLM in the cancer cell is a result of one or more amino acid deletions in a protein encoded by a BLM gene. In some embodiments, the decreased level and/or activity of BLM in the cancer cell is a result of one or more inactivating amino acid substitutions in a protein encoded by a BLM gene.

In some embodiments, the increased cGAS/STING signaling pathway activity is a result of a decreased level and/or activity of PARP1 in the cancer cell. In some embodiments, the decreased level and/or activity of PARP1 in the cancer cell is a result of a frameshift mutation in a PARP1 gene. In some embodiments, the frameshift mutation in a PARP1 gene is a S507Afs*17 frameshift deletion (e.g., a mutation in a PARP1 gene that causes a S507Afs*17 frameshift deletion with respect to SEQ ID NO: 43). In some embodiments, the decreased level and/or activity of PARP1 in the cancer cell is a result of PARP1 gene loss in the cancer cell. In some embodiments, the decreased level and/or activity of PARP1 in the cancer cell is a result of one or more amino acid deletions in a protein encoded by a PARP1 gene. In some embodiments, the decreased level and/or activity of PARP1 in the cancer cell is a result of one or more inactivating amino acid substitutions in a protein encoded by a PARP1 gene.

In some embodiments, the increased cGAS/STING signaling pathway activity is a result of a decreased level and/or activity of RPA1 in the cancer cell. In some embodiments, the decreased level and/or activity of RPA1 in the cancer cell is a result of a mutation that results in aberrant RPA1 mRNA splicing in the cancer cell. In some embodiments, the mutation that results in aberrant RPA1 mRNA splicing in the cancer cell is a X12 splice mutation. In some embodiments, the decreased level and/or activity of RPA1 in the cancer cell is a result of RPA1 gene loss in the cancer cell. In some embodiments, the decreased level and/or activity of RPA1 in the cancer cell is a result of one or more amino acid deletions in a protein encoded by a RPA1 gene. In some embodiments, the decreased level and/or activity of RPA1 in the cancer cell is a result of one or more inactivating amino acid substitutions in a protein encoded by a RPA1 gene.

In some embodiments, the increased cGAS/STING signaling pathway activity is a result of a decreased level and/or activity of RAD51 in the cancer cell. In some embodiments, the decreased level and/or activity of RAD51 in the cancer cell is a result of one or more inactivating amino acid substitutions in a protein encoded by a RAD51 gene. In some embodiments, the one or more inactivating amino acid substitutions in a protein encoded by a RAD51 gene is an R254* amino acid substitution (e.g., a mutation in a RAD51 gene that causes a R254* amino acid substitution with respect to SEQ ID NO: 51). In some embodiments, the decreased level and/or activity of RAD51 in the cancer cell is a result of RAD51 gene loss in the cancer cell. In some embodiments, the decreased level and/or activity of RAD51 in the cancer cell is a result of one or more amino acid deletions in a protein encoded by a RAD51 gene.

In some embodiments, the increased cGAS/STING signaling pathway activity is a result of an increased level and/or activity of MUS81 in the cancer cell. In some embodiments, the increased level and/or activity of MUS81 in the cancer cell is a result of MUS81 gene amplification in the cancer cell. In some embodiments, increased level and/or activity of MUS81 in the cancer cell is a result of one or more activating amino acid substitutions in a protein encoded by a MUS81 gene.

In some embodiments, increased cGAS/STING signaling pathway activity is a result of an increased level and/or activity of IFI16 in the cancer cell. In some embodiments, the increased level and/or activity of IFI16 in the cancer cell is a result of IFI16 gene amplification in the cancer cell. In some embodiments, increased level and/or activity of IFI16 in the cancer cell is a result of one or more activating amino acid substitutions in a protein encoded by an IFI16 gene.

In some embodiments, the increased cGAS/STING signaling pathway activity is a result of an increased level and/or activity of cGAS in the cancer cell. In some embodiments, the increased level and/or activity of cGAS in the cancer cell is a result of cGAS gene amplification in the cancer cell. In some embodiments, the increased level and/or activity of cGAS in the cancer cell is a result of one or more activating amino acid substitutions in a protein encoded by a cGAS gene.

In some embodiments, the increased cGAS/STING signaling pathway activity is a result of an increased activity of STING in the cancer cell. In some embodiments, the increased activity of STING in the cancer cell is a result of one or more activating amino acid substitutions in a protein encoded by a STING gene.

In some embodiments, the increased cGAS/STING signaling pathway activity is a result of an increased level and/or activity of DDX41 in the cancer cell. In some embodiments, the increased level and/or activity of DDX41 in the cancer cell is a result of DDX41 gene amplification in the cancer cell. In some embodiments, the increased level and/or activity of DDX41 in the cancer cell is a result of one or more activating amino acid substitutions in a protein encoded by a DDX41 gene.

In some embodiments, the increased cGAS/STING signaling pathway activity is a result of an increased level and/or activity of EXO1 in the cancer cell. In some embodiments, the increased level and/or activity of EXO1 in the cancer cell is a result of EXO1 gene amplification in the cancer cell. In some embodiments, increased level and/or activity of EXO1 in the cancer cell is a result of one or more activating amino acid substitutions in a protein encoded by a EXO1 gene.

In some embodiments, the increased cGAS/STING signaling pathway activity is a result of an increased level and/or activity of DNA2 in the cancer cell. In some embodiments, the increased level and/or activity of DNA2 in the cancer cell is a result of DNA2 gene amplification in the cancer cell. In some embodiments, the increased level and/or activity of DNA2 in the cancer cell is a result of one or more activating amino acid substitutions in a protein encoded by a DNA2 gene.

In some embodiments, the increased cGAS/STING signaling pathway activity is a result of an increased level and/or activity of RBBP8 (CtIP) in the cancer cell. In some embodiments, the increased level and/or activity of RBBP8 (CtIP) in the cancer cell is a result of RBBP8 (CtIP) gene amplification in the cancer cell. In some embodiments, the increased level and/or activity of RBBP8 (CtIP) in the cancer cell is a result of one or more activating amino acid substitutions in a protein encoded by a RBBP8 (CtIP) gene.

In some embodiments, the increased cGAS/STING signaling pathway activity is a result of an increased level and/or activity of MRE11 in the cancer cell. In some embodiments, the increased level and/or activity of MRE11 in the cancer cell is a result of MRE11 gene amplification in the cancer cell. In some embodiments, the increased level and/or activity of MRE11 in the cancer cell is a result of one or more activating amino acid substitutions in a protein encoded by a MRE11 gene.

In some embodiments, the subject has been diagnosed or identified as having a cancer. In some embodiments, the cancer is selected from the group consisting of: renal clear cell carcinoma, uveal melanoma, tongue squamous cell carcinoma, breast cancer, and skin cancer.

In some embodiments of any of the methods described herein, the STING antagonist is an inhibitory nucleic acid (e.g., a short interfering RNA, an antisense nucleic acid, a cyclic dinucleotide, or a ribozyme). In some embodiments of any of the methods described herein, the STING antagonist or cGAS inhibitor is any of the compounds described herein, or a pharmaceutically acceptable salt, solvate, or co-crystal thereof.

Additional exemplary aspects that can be used or incorporated in these methods are described herein.

### Methods of Selecting a Subject for Participation in a Clinical Trial

Provided herein are methods of selecting a subject (e.g., any of the exemplary subjects described herein) for participation in a clinical trial that include: (a) identifying a subject having a cancer cell having one or both of (i) decreased TREX1 level and/or activity (e.g., a decrease of about 1% to about 99%, or any subranges of this range described herein) (e.g., as compared to a reference level), and (ii) increased cGAS/STING signaling pathway activity (e.g., an increase of between 1% and 1000%, or any of the subranges of this range described herein) (e.g., as compared to a reference level); and/or identifying a subject identified as having an elevated level of cGAMP in a serum or a tumor sample (e.g., an increase of between 1% and 1000%, or any of the subranges of this range described herein) (e.g., as compared to a reference level); and (b) selecting the identified subject for participation in a clinical trial that comprises administration of a treatment comprising a therapeutically effective amount of a STING antagonist or cGAS inhibitor (e.g., any of the exemplary STING antagonists or cGAS inhibitors described herein) or a pharmaceutically acceptable salt, solvate, or co-crystal thereof.

Also provided herein are methods of selecting a subject (e.g., any of the exemplary subjects described herein) for participation in a clinical trial that include: selecting a subject identified as having a cell (e.g., a cancer cell) having one or both of (i) decreased TREX1 level and/or activity (e.g., a decrease of about 1% to about 99%, or any subranges of this range described herein) (e.g., as compared to a reference level), and (ii) increased cGAS/STING signaling pathway activity (e.g., an increase of between 1% and 1000%, or any of the subranges of this range described herein) (e.g., as compared to a reference level) and/or selecting a subject identified as having an elevated level of cGAMP in a serum or a tumor sample (e.g., an increase of between 1% and 1000%, or any of the subranges of this range described herein) (e.g., as compared to a reference level) for participation in a clinical trial that comprises administration of a treatment comprising a therapeutically effective amount of an STING antagonist or cGAS inhibitor (e.g., any of the exemplary STING antagonists or cGAS inhibitors described herein) or a pharmaceutically acceptable salt, solvate, or co-crystal thereof.

In some embodiments, the subject is identified as having a cancer cell having decreased TREX1 level and/or activity. In some embodiments, the subject is identified as having an elevated level of cGAMP in a serum or a tumor sample. In some embodiments, the subject is identified as having a cancer cell having increased cGAS/STING signaling pathway activity. In some embodiments, the subject is identified having a cancer cell having both (i) decreased TREX1 level and/or activity and (ii) increased cGAS/STING signaling pathway activity (e.g., an increase of between 1% and 1000%, or any of the subranges of this range described herein) (e.g., as compared to a reference level) or to a subject identified as having an elevated level of cGAMP in a serum or tumor sample (e.g., an increase of between 1% and 1000%, or any of the subranges of this range described herein) (e.g., as compared to a reference level). In some embodiments, the subject is identified as having a cancer cell having decreased TREX1 level. In some embodiments, the TREX1 level is a level of TREX1 protein in the cancer cell. In some embodiments, the identification of the subject as having a cancer cell having a decreased TREX1 level includes detecting a decreased level of TREX1 protein in the cancer cell. In some embodiments, the TREX1 level is a level of TREX1 mRNA in the cancer cell. In some embodiments, the identification of the subject as having a cancer cell having a decreased TREX1 level comprises detecting a decreased level of TREX1 mRNA in the cancer cell.

In some embodiments, the decreased TREX1 level and/or activity is a result of TREX1 gene loss in the cancer cell. In some embodiments, the TREX1 gene loss is loss of one allele of the TREX1 gene. In some embodiments, the TREX1 gene loss is loss of both alleles of the TREX1 gene. In some embodiments, the identification of the subject as having a cancer cell having decreased TREX1 level and/or activity comprises detecting TREX1 gene loss in the cancer cell. In some embodiments, the decreased TREX1 level and/or activity is a result of one or more amino acid deletions in a protein encoded by a TREX1 gene in the cancer cell. In some embodiments, the identification of the subject as having a cancer cell having decreased TREX1 level and/or activity comprises detecting one or more amino acid deletions in a protein encoded by a TREX1 gene in the cancer cell. In some embodiments, the decreased TREX1 level and/or activity is a result of one or more inactivating amino acid substitutions in a protein encoded by a TREX1 gene in the cancer cell. In some embodiments, the identification of the subject as having a cancer cell having decreased TREX1 expression and/or activity comprises detecting one or more inactivating amino acid substitutions in a protein encoded by a TREX1 gene in the cancer cell.

In some embodiments, the increased cGAS/STING signaling pathway activity is a result of a decreased level and/or activity of BRCA1 in the cancer cell. In some embodiments, the decreased level and/or activity of BRCA1 in the cancer cell is a result of a frameshift mutation in a BRCA1 gene. In some embodiments, frameshift mutation in a BRCA1 gene is a E111Gfs*3 frameshift insertion (e.g., a mutation in a BRCA1 gene that causes a E111Gfs*3 frameshift insertion with respect to SEQ ID NO: 15). In some embodiments, the decreased level and/or activity of BRCA1 in the cancer cell is a result of BRCA1 gene loss in the cancer cell. In some embodiments, the decreased level and/or activity of BRCA1 in the cancer cell is a result of one or more amino acid deletions in a protein encoded by a BRCA1 gene. In some embodiments, the decreased level and/or activity of BRCA1 in the cancer cell is a result of one or more inactivating amino acid substitutions in a protein encoded by a BRCA1 gene.

In some embodiments, the increased cGAS/STING signaling pathway activity is a result of a decreased level and/or activity of BRCA2 gene. In some embodiments, the decreased level and/or activity of BRCA2 in the cancer cell is a result of a frameshift mutation in a BRCA2 gene. In some embodiments, the frameshift mutation in a BRCA2 gene is a N1784Kfs*3 frameshift insertion (e.g., a mutation in a BRCA2 gene that causes a N1784Kfs*3 frameshift insertion with respect to SEQ ID NO: 25). In some embodiments, the decreased level and/or activity of BRCA2 in the cancer cell is a result of BRCA2 gene loss in the cancer cell. In some embodiments, the decreased level and/or activity of BRCA2 in the cancer cell is a result of one or more amino acid deletions in a protein encoded by a BRCA2 gene. In some embodiments, decreased level and/or activity of BRCA2 in the cancer cell is a result of one or more inactivating amino acid substitutions in a protein encoded by a BRCA2 gene.

In some embodiments, the increased cGAS/STING signaling pathway activity is a result of a decreased level and/or activity of SAMHD1 in the cancer cell. In some embodiments, the decreased level and/or activity of SAMHD1 in the cancer cell is a result of one or more inactivating amino acid substitutions in a protein encoded by a SAMHD1 gene in the cancer cell. In some embodiments, the one or more inactivating amino acid substitutions in a protein encoded by a SAMHD1 gene is a V133I amino acid substitution (e.g., a mutation in a SAMHD1 gene that causes a V133I amino acid substitution with respect to SEQ ID NO: 27). In some embodiments, the decreased level and/or activity of SAMHD1 in the cancer cell is a result of SAMHD1 gene loss in the cancer cell. In some embodiments, the decreased level and/or activity of SAMHD1 in the cancer cell is a result of one or more amino acid deletions in a protein encoded by a SAMHD1 gene.

In some embodiments, the increased cGAS/STING signaling pathway activity is a result of a decreased level and/or activity of DNASE2 in the cancer cell. In some embodiments, the decreased level and/or activity of DNASE2 in the cancer cell is a result of one or more inactivating amino acid substitutions in a protein encoded by a DNASE2 gene in the cancer cell. In some embodiments, the one or more inactivating amino acid substitutions in a protein encoded by a DNASE2 gene is a R314W amino acid substitution (e.g., a mutation in a DNASE2 gene that causes a R314W amino acid substitution with respect to SEQ ID NO: 33). In some embodiments, the decreased level and/or activity of DNASE2 in the cancer cell is a result of DNASE2 gene loss in the cancer cell. In some embodiments, the decreased level and/or activity of DNASE2 in the cancer cell is a result of one or more amino acid deletions in a protein encoded by a DNASE2 gene.

In some embodiments, the increased cGAS/STING signaling pathway activity is a result of a decreased level and/or activity of BLM in the cancer cell. In some embodiments, the decreased level and/or activity of BLM in the cancer cell is a result of a frameshift mutation in a BLM gene. In some embodiments, the frameshift mutation in a BLM gene is a N515Mfs*16 frameshift deletion (e.g., a mutation in a BLM gene that causes a N515Mfs*16 frameshift deletion with respect to SEQ ID NO: 37). In some embodiments, the decreased level and/or activity of BLM in the cancer cell is a result of BLM gene loss in the cancer cell. In some embodiments, the decreased level and/or activity of BLM in the cancer cell is a result of one or more amino acid deletions in a protein encoded by a BLM gene. In some embodiments, the decreased level and/or activity of BLM in the cancer cell is a result of one or more inactivating amino acid substitutions in a protein encoded by a BLM gene.

In some embodiments, the increased cGAS/STING signaling pathway activity is a result of a decreased level and/or activity of PARP1 in the cancer cell. In some embodiments, the decreased level and/or activity of PARP1 in the cancer cell is a result of a frameshift mutation in a PARP1 gene. In some embodiments, the frameshift mutation in a PARP1 gene is a S507Afs*17 frameshift deletion (e.g., a mutation in a PARP1 gene that causes a S507Afs*17 frameshift deletion with respect to SEQ ID NO: 43). In some embodiments, the decreased level and/or activity of PARP1 in the cancer cell is a result of PARP1 gene loss in the cancer cell. In some embodiments, the decreased level and/or activity of PARP1 in the cancer cell is a result of one or more amino acid deletions in a protein encoded by a PARP1 gene. In some embodiments, the decreased level and/or activity of PARP1 in the cancer cell is a result of one or more inactivating amino acid substitutions in a protein encoded by a PARP1 gene.

In some embodiments, the increased cGAS/STING signaling pathway activity is a result of a decreased level and/or activity of RPA1 in the cancer cell. In some embodiments, the decreased level and/or activity of RPA1 in the cancer cell is a result of a mutation that results in aberrant RPA1 mRNA splicing in the cancer cell. In some embodiments, the mutation that results in aberrant RPA1 mRNA splicing in the cancer cell is a X12 splice mutation. In some embodiments, the decreased level and/or activity of RPA1 in the cancer cell is a result of RPA1 gene loss in the cancer cell. In some embodiments, the decreased level and/or activity of RPA1 in the cancer cell is a result of one or more amino acid deletions in a protein encoded by a RPA1 gene. In some embodiments, the decreased level and/or activity of RPA1 in the cancer cell is a result of one or more inactivating amino acid substitutions in a protein encoded by a RPA1 gene.

In some embodiments, the increased cGAS/STING signaling pathway activity is a result of a decreased level and/or activity of RAD51 in the cancer cell. In some embodiments, the decreased level and/or activity of RAD51 in the cancer cell is a result of one or more inactivating amino acid substitutions in a protein encoded by a RAD51 gene. In some embodiments, the one or more inactivating amino acid substitutions in a protein encoded by a RAD51 gene is an R254* amino acid substitution (e.g., a mutation in a RAD51 gene that causes a R254* amino acid substitution with respect to SEQ ID NO: 51). In some embodiments, the decreased level and/or activity of RAD51 in the cancer cell is a result of RAD51 gene loss in the cancer cell. In some embodiments, the decreased level and/or activity of RAD51 in the cancer cell is a result of one or more amino acid deletions in a protein encoded by a RAD51 gene.

In some embodiments, the increased cGAS/STING signaling pathway activity is a result of an increased level and/or activity of MUS81 in the cancer cell. In some embodiments, the increased level and/or activity of MUS81 in the cancer cell is a result of MUS81 gene amplification in the cancer cell. In some embodiments, increased level and/or activity of MUS81 in the cancer cell is a result of one or more activating amino acid substitutions in a protein encoded by a MUS81 gene.

In some embodiments, increased cGAS/STING signaling pathway activity is a result of an increased level and/or activity of IFI16 in the cancer cell. In some embodiments, the increased level and/or activity of IFI16 in the cancer cell is a result of IFI16 gene amplification in the cancer cell. In some embodiments, increased level and/or activity of IFI16 in the cancer cell is a result of one or more activating amino acid substitutions in a protein encoded by a IFI16 gene.

In some embodiments, the increased cGAS/STING signaling pathway activity is a result of an increased level and/or activity of cGAS in the cancer cell. In some embodiments, the increased level and/or activity of cGAS in the cancer cell is a result of cGAS gene amplification in the cancer cell. In some embodiments, the increased level and/or activity of cGAS in the cancer cell is a result of one or more activating amino acid substitutions in a protein encoded by a cGAS gene.

In some embodiments, the increased STING signaling pathway activity is a result of an increased activity of STING in the cancer cell. In some embodiments, the increased activity of STING in the cancer cell is a result of one or more activating amino acid substitutions in a protein encoded by a STING gene.

In some embodiments, the increased cGAS/STING signaling pathway activity is a result of an increased level and/or activity of DDX41 in the cancer cell. In some embodiments, the increased level and/or activity of DDX41 in the cancer cell is a result of DDX41 gene amplification in the cancer cell. In some embodiments, the increased level and/or activity of DDX41 in the cancer cell is a result of one or more activating amino acid substitutions in a protein encoded by a DDX41 gene.

In some embodiments, the increased cGAS/STING signaling pathway activity is a result of an increased level and/or activity of EXO1 in the cancer cell. In some embodiments, the increased level and/or activity of EXO1 in the cancer cell is a result of EXO1 gene amplification in the cancer cell. In some embodiments, increased level and/or activity of EXO1 in the cancer cell is a result of one or more activating amino acid substitutions in a protein encoded by a EXO1 gene.

In some embodiments, the increased cGAS/STING signaling pathway activity is a result of an increased level and/or activity of DNA2 in the cancer cell. In some embodiments, the increased level and/or activity of DNA2 in the cancer cell is a result of DNA2 gene amplification in the cancer cell. In some embodiments, the increased level and/or activity of DNA2 in the cancer cell is a result of one or more activating amino acid substitutions in a protein encoded by a DNA2 gene.

In some embodiments, the increased cGAS/STING signaling pathway activity is a result of an increased level and/or activity of RBBP8 (CtIP) in the cancer cell. In some embodiments, the increased level and/or activity of RBBP8 (CtIP) in the cancer cell is a result of RBBP8 (CtIP) gene amplification in the cancer cell. In some embodiments, the increased level and/or activity of RBBP8 (CtIP) in the cancer cell is a result of one or more activating amino acid substitutions in a protein encoded by a RBBP8 (CtIP) gene.

In some embodiments, the increased cGAS/STING signaling pathway activity is a result of an increased level and/or activity of MRE11 in the cancer cell. In some embodiments, the increased level and/or activity of MRE11 in the cancer cell is a result of MRE11 gene amplification in the cancer cell. In some embodiments, the increased level and/or activity of MRE11 in the cancer cell is a result of one or more activating amino acid substitutions in a protein encoded by a MRE11 gene.

In some embodiments, the subject has been diagnosed or identified as having a cancer. In some embodiments, the cancer is selected from the group consisting of: renal clear cell carcinoma, uveal melanoma, tongue squamous cell carcinoma, breast cancer, and skin cancer.

In some embodiments of any of the methods described herein, the STING antagonist is an inhibitory nucleic acid (e.g., a short interfering RNA, an antisense nucleic acid, a cyclic dinucleotide, or a ribozyme). In some embodiments of any of the methods described herein, the STING antagonist or cGAS inhibitor is any of the compounds described herein, or a pharmaceutically acceptable salt, solvate, or co-crystal thereof.

Additional exemplary aspects that can be used or incorporated in these methods are described herein.

Methods of Predicting a Subject's Responsiveness to a STING Antagonist or cGAS Inhibitor

Provided herein are methods of predicting a subject's (e.g., any of the exemplary subjects described herein) responsiveness to a compound of any one of Formulas I-X that include: (a) determining that a subject has a cancer cell having one or both of (i) decreased TREX1 level and/or activity (e.g., a decrease of about 1% to about 99%, or any subranges of this range described herein) (e.g., as compared to a reference level), and (ii) increased cGAS/STING signaling pathway activity (e.g., an increase of between 1% and 1000%, or any of the subranges of this range described herein) (e.g., as compared to a reference level) or to a subject identified as having an elevated level of cGAMP in a serum or a tumor sample (e.g., an increase of between 1% and 1000%, or any of the subranges of this range described herein) (e.g., as compared to a reference level); and (b) identifying that the subject determined to have one or both of (i) decreased TREX1 level and/or activity (e.g., a decrease of about 1% to about 99%, or any subranges of this range described herein) (e.g., as compared to a reference level), and (ii) increased cGAS/STING signaling pathway activity (e.g., an increase of between 1% and 1000%, or any of the subranges of this range described herein) (e.g., as compared to a reference level) or to a subject identified as having an elevated level of cGAMP in a serum or a tumor sample (e.g., an increase of between 1% and 1000%, or any of the subranges of this range described herein) (e.g., as compared to a reference level) in step (a) has an increased likelihood of being responsive to treatment with a compound of any one of Formulas I-X.

Provided herein are methods of predicting a subject's (e.g., any of the exemplary subjects described herein) responsiveness to a STING antagonist or cGAS inhibitor that include: (a) determining that a subject has a cancer cell having one or both of (i) decreased TREX1 level and/or activity (e.g., a decrease of about 1% to about 99%, or any subranges of this range described herein) (e.g., as compared to a reference level), and (ii) increased cGAS/STING signaling pathway activity (e.g., an increase of between 1% and 1000%, or any of the subranges of this range described herein) (e.g., as compared to a reference level) or to a subject identified as having an elevated level of cGAMP in a serum or a tumor sample (e.g., an increase of between 1% and 1000%, or any of the subranges of this range described herein) (e.g., as compared to a reference level); and (b) identifying that the subject determined to have one or both of (i) decreased TREX1 level and/or activity (e.g., a decrease of about 1% to about 99%, or any subranges of this range described herein) (e.g., as compared to a reference level), and (ii) increased cGAS/STING signaling pathway activity (e.g., an increase of between 1% and 1000%, or any of the subranges of this range described herein) (e.g., as compared to a reference level) or to a subject identified as having an elevated level of cGAMP in a serum or a tumor sample (e.g., an increase of between 1% and 1000%, or any of the subranges of this range described herein) (e.g., as compared to a reference level) in step (a) has an increased likelihood of being responsive to treatment with a STING antagonist or a cGAS inhibitor.

Also provided herein are methods of predicting a subject's (e.g., any of the exemplary subjects described herein) responsiveness to a compound of any one of Formulas I-X that include: identifying a subject determined to have a cell (e.g., a cancer cell) having one or both of (i) decreased TREX1 level and/or activity (e.g., a decrease of about 1% to about 99%, or any subranges of this range described herein) (e.g., as compared to a reference level), and (ii) increased cGAS/STING signaling pathway activity (e.g., an increase of between 1% and 1000%, or any of the subranges of this range described herein) (e.g., as compared to a reference level) or to a subject identified as having an elevated level of cGAMP in a serum or a tumor sample (e.g., an increase of between 1% and 1000%, or any of the subranges of this range described herein) (e.g., as compared to a reference level) as having an increased likelihood of being responsive to treatment with a compound of any one of Formulas I-X.

Also provided herein are methods of predicting a subject's (e.g., any of the exemplary subjects described herein) responsiveness to a STING antagonist or a cGAS inhibitor that include: identifying a subject determined to have a cell (e.g., a cancer cell) having one or both of (i) decreased TREX1 level and/or activity (e.g., a decrease of about 1% to about 99%, or any subranges of this range described herein) (e.g., as compared to a reference level), and (ii) increased cGAS/STING signaling pathway activity (e.g., an increase of between 1% and 1000%, or any of the subranges of this range described herein) (e.g., as compared to a reference level) or to a subject identified as having an elevated level of cGAMP in a serum or a tumor sample (e.g., an increase of between 1% and 1000%, or any of the subranges of this range described herein) (e.g., as compared to a reference level) as having an increased likelihood of being responsive to treatment with a STING antagonist or a cGAS inhibitor.

In some embodiments, the subject is identified as having a cancer cell having decreased TREX1 level and/or activity. In some embodiments, the subject is identified as having a cancer cell having increased cGAS/STING signaling pathway activity. In some embodiments, the subject is identified having a cancer cell having both (i) decreased TREX1 level and/or activity and (ii) increased cGAS/STING signaling pathway activity (e.g., an increase of between 1% and 1000%, or any of the subranges of this range described herein) (e.g., as compared to a reference level) or to a subject identified as having an elevated level of cGAMP in a serum or a tumor sample (e.g., an increase of between 1% and 1000%, or any of the subranges of this range described herein) (e.g., as compared to a reference level). In some embodiments, the subject is identified as having a cancer cell having decreased TREX1 level. In some embodiments, the TREX1 level is a level of TREX1 protein in the cancer cell. In some embodiments, the identification of the subject as having a cancer cell having a decreased TREX1 level includes detecting a decreased level of TREX1 protein in the cancer cell. In some embodiments, the TREX1 level is a level of TREX1 mRNA in the cancer cell. In some embodiments, the identification of the subject as having a cancer cell having a decreased TREX1 level comprises detecting a decreased level of TREX1 mRNA in the cancer cell.

In some embodiments, the decreased TREX1 level and/or activity is a result of TREX1 gene loss in the cancer cell. In some embodiments, the TREX1 gene loss is loss of one allele of the TREX1 gene. In some embodiments, the TREX1 gene loss is loss of both alleles of the TREX1 gene. In some embodiments, the identification of the subject as having a cancer cell having decreased TREX1 level and/or activity comprises detecting TREX1 gene loss in the cancer cell. In some embodiments, the decreased TREX1 level and/or activity is a result of one or more amino acid deletions in a protein encoded by a TREX1 gene in the cancer cell. In some embodiments, the identification of the subject as having a cancer cell having decreased TREX1 level and/or activity comprises detecting one or more amino acid deletions in a protein encoded by a TREX1 gene in the cancer cell. In some embodiments, the decreased TREX1 level and/or activity is a result of one or more inactivating amino acid substitutions in a protein encoded by a TREX1 gene in the cancer cell. In some embodiments, the identification of the subject as having a cancer cell having decreased TREX1 expression and/or activity comprises detecting one or more inactivating amino acid substitutions in a protein encoded by a TREX1 gene in the cancer cell.

In some embodiments, the increased cGAS/STING signaling pathway activity is a result of a decreased level and/or activity of BRCA1 in the cancer cell. In some embodiments, the decreased level and/or activity of BRCA1 in the cancer cell is a result of a frameshift mutation in a BRCA1 gene. In some embodiments, frameshift mutation in a BRCA1 gene is a E111Gfs*3 frameshift insertion (e.g., a mutation in a BRCA1 gene that causes a E111Gfs*3 frameshift insertion with respect to SEQ ID NO: 15). In some embodiments, the decreased level and/or activity of BRCA1 in the cancer cell is a result of BRCA1 gene loss in the cancer cell. In some embodiments, the decreased level and/or activity of BRCA1 in the cancer cell is a result of one or more amino acid deletions in a protein encoded by a BRCA1 gene. In some embodiments, the decreased level and/or activity of BRCA1 in the cancer cell is a result of one or more inactivating amino acid substitutions in a protein encoded by a BRCA1 gene.

In some embodiments, the increased cGAS/STING signaling pathway activity is a result of a decreased level and/or activity of BRCA2 gene. In some embodiments, the decreased level and/or activity of BRCA2 in the cancer cell is a result of a frameshift mutation in a BRCA2 gene. In some embodiments, the frameshift mutation in a BRCA2 gene is a N1784Kfs*3 frameshift insertion (e.g., a mutation in a BRCA2 gene that causes a N1784Kfs*3 frameshift insertion with respect to SEQ ID NO: 25). In some embodiments, the decreased level and/or activity of BRCA2 in the cancer cell is a result of BRCA2 gene loss in the cancer cell. In some embodiments, the decreased level and/or activity of BRCA2 in the cancer cell is a result of one or more amino acid deletions in a protein encoded by a BRCA2 gene. In some embodiments, decreased level and/or activity of BRCA2 in the cancer cell is a result of one or more inactivating amino acid substitutions in a protein encoded by a BRCA2 gene.

In some embodiments, the increased cGAS/STING signaling pathway activity is a result of a decreased level and/or activity of SAMHD1 in the cancer cell. In some embodiments, the decreased level and/or activity of SAMHD1 in the cancer cell is a result of one or more inactivating amino acid substitutions in a protein encoded by a SAMHD1 gene in the cancer cell. In some embodiments, the one or more inactivating amino acid substitutions in a protein encoded by a SAMHD1 gene is a V133I amino acid substitution (e.g., a mutation in a SAMHD1 gene that causes a V133I amino acid substitution with respect to SEQ ID NO: 27). In some embodiments, the decreased level and/or activity of SAMHD1 in the cancer cell is a result of SAMHD1 gene loss in the cancer cell. In some embodiments, the decreased level and/or activity of SAMHD1 in the cancer cell is a result of one or more amino acid deletions in a protein encoded by a SAMHD1 gene.

In some embodiments, the increased cGAS/STING signaling pathway activity is a result of a decreased level and/or activity of DNASE2 in the cancer cell. In some embodiments, the decreased level and/or activity of DNASE2 in the cancer cell is a result of one or more inactivating amino acid substitutions in a protein encoded by a DNASE2 gene in the cancer cell. In some embodiments, the one or more inactivating amino acid substitutions in a protein encoded by a DNASE2 gene is a R314W amino acid substitution (e.g., a mutation in a DNASE2 gene that causes a R314W amino acid substitution with respect to SEQ ID NO: 33). In some embodiments, the decreased level and/or activity of DNASE2 in the cancer cell is a result of DNASE2 gene loss in the cancer cell. In some embodiments, the decreased level and/or activity of DNASE2 in the cancer cell is a result of one or more amino acid deletions in a protein encoded by a DNASE2 gene.

In some embodiments, the increased cGAS/STING signaling pathway activity is a result of a decreased level and/or activity of BLM in the cancer cell. In some embodiments, the decreased level and/or activity of BLM in the cancer cell is a result of a frameshift mutation in a BLM gene. In some embodiments, the frameshift mutation in a BLM gene is a N515Mfs*16 frameshift deletion (e.g., a mutation in a BLM gene that causes a N515Mfs*16 frameshift deletion with respect to SEQ ID NO: 37). In some embodiments, the decreased level and/or activity of BLM in the cancer cell is a result of BLM gene loss in the cancer cell. In some embodiments, the decreased level and/or activity of BLM in the cancer cell is a result of one or more amino acid deletions in a protein encoded by a BLM gene. In some embodiments, the decreased level and/or activity of BLM in the cancer cell is a result of one or more inactivating amino acid substitutions in a protein encoded by a BLM gene.

In some embodiments, the increased cGAS/STING signaling pathway activity is a result of a decreased level and/or activity of PARP1 in the cancer cell. In some embodiments, the decreased level and/or activity of PARP1 in the cancer cell is a result of a frameshift mutation in a PARP1 gene. In some embodiments, the frameshift mutation in a PARP1 gene is a S507Afs*17 frameshift deletion (e.g., a mutation in a PARP1 gene that causes a S507Afs*17 frameshift deletion with respect to SEQ ID NO: 43). In some embodiments, the decreased level and/or activity of PARP1 in the cancer cell is a result of PARP1 gene loss in the cancer cell. In some embodiments, the decreased level and/or activity of PARP1 in the cancer cell is a result of one or more amino acid deletions in a protein encoded by a PARP1 gene. In some embodiments, the decreased level and/or activity of PARP1 in the cancer cell is a result of one or more inactivating amino acid substitutions in a protein encoded by a PARP1 gene.

In some embodiments, the increased cGAS/STING signaling pathway activity is a result of a decreased level and/or activity of RPA1 in the cancer cell. In some embodiments, the decreased level and/or activity of RPA1 in the cancer cell is a result of a mutation that results in aberrant RPA1 mRNA splicing in the cancer cell. In some embodiments, the mutation that results in aberrant RPA1 mRNA splicing in the cancer cell is a X12 splice mutation. In some embodiments, the decreased level and/or activity of RPA1 in the cancer cell is a result of RPA1 gene loss in the cancer cell. In some embodiments, the decreased level and/or activity of RPA1 in the cancer cell is a result of one or more amino acid deletions in a protein encoded by a RPA1 gene. In some embodiments, the decreased level and/or activity of RPA1 in the cancer cell is a result of one or more inactivating amino acid substitutions in a protein encoded by a RPA1 gene.

In some embodiments, the increased cGAS/STING signaling pathway activity is a result of a decreased level and/or activity of RAD51 in the cancer cell. In some embodiments, the decreased level and/or activity of RAD51 in the cancer cell is a result of one or more inactivating amino acid substitutions in a protein encoded by a RAD51 gene. In some embodiments, the one or more inactivating amino acid substitutions in a protein encoded by a RAD51 gene is an R254* amino acid substitution (e.g., a mutation in a RAD51 gene that causes a R254* amino acid substitution with respect to SEQ ID NO: 51). In some embodiments, the decreased level and/or activity of RAD51 in the cancer cell is a result of RAD51 gene loss in the cancer cell. In some embodiments, the decreased level and/or activity of RAD51 in the cancer cell is a result of one or more amino acid deletions in a protein encoded by a RAD51 gene.

In some embodiments, the increased cGAS/STING signaling pathway activity is a result of an increased level and/or activity of MUS81 in the cancer cell. In some embodiments, the increased level and/or activity of MUS81 in the cancer cell is a result of MUS81 gene amplification in the cancer cell. In some embodiments, increased level and/or activity of MUS81 in the cancer cell is a result of one or more activating amino acid substitutions in a protein encoded by a MUS81 gene.

In some embodiments, increased cGAS/STING signaling pathway activity is a result of an increased level and/or activity of IFI16 in the cancer cell. In some embodiments, the increased level and/or activity of IFI16 in the cancer cell is a result of IFI16 gene amplification in the cancer cell. In some embodiments, increased level and/or activity of IFI16 in the cancer cell is a result of one or more activating amino acid substitutions in a protein encoded by a IFI16 gene.

In some embodiments, the increased cGAS/STING signaling pathway activity is a result of an increased level and/or activity of cGAS in the cancer cell. In some embodiments, the increased level and/or activity of cGAS in the cancer cell is a result of cGAS gene amplification in the cancer cell. In some embodiments, the increased level and/or activity of cGAS in the cancer cell is a result of one or more activating amino acid substitutions in a protein encoded by a cGAS gene.

In some embodiments, the increased STING signaling pathway activity is a result of an increased activity of STING in the cancer cell. In some embodiments, the increased activity of STING in the cancer cell is a result of one or more activating amino acid substitutions in a protein encoded by a STING gene.

In some embodiments, the increased cGAS/STING signaling pathway activity is a result of an increased level and/or activity of DDX41 in the cancer cell. In some embodiments, the increased level and/or activity of DDX41 in the cancer cell is a result of DDX41 gene amplification in the cancer cell. In some embodiments, the increased level and/or activity of DDX41 in the cancer cell is a result of one or more activating amino acid substitutions in a protein encoded by a DDX41 gene.

In some embodiments, the increased cGAS/STING signaling pathway activity is a result of an increased level and/or activity of EXO1 in the cancer cell. In some embodiments, the increased level and/or activity of EXO1 in the cancer cell is a result of EXO1 gene amplification in the cancer cell. In some embodiments, increased level and/or activity of EXO1 in the cancer cell is a result of one or more activating amino acid substitutions in a protein encoded by a EXO1 gene.

In some embodiments, the increased cGAS/STING signaling pathway activity is a result of an increased level and/or activity of DNA2 in the cancer cell. In some embodiments, the increased level and/or activity of DNA2 in the cancer cell is a result of DNA2 gene amplification in the cancer cell. In some embodiments, the increased level and/or activity of DNA2 in the cancer cell is a result of one or more activating amino acid substitutions in a protein encoded by a DNA2 gene.

In some embodiments, the increased cGAS/STING signaling pathway activity is a result of an increased level and/or activity of RBBP8 (CtIP) in the cancer cell. In some embodiments, the increased level and/or activity of RBBP8 (CtIP) in the cancer cell is a result of RBBP8 (CtIP) gene amplification in the cancer cell. In some embodiments, the increased level and/or activity of RBBP8 (CtIP) in the cancer cell is a result of one or more activating amino acid substitutions in a protein encoded by a RBBP8 (CtIP) gene.

In some embodiments, the increased cGAS/STING signaling pathway activity is a result of an increased level and/or activity of MRE11 in the cancer cell. In some embodiments, the increased level and/or activity of MRE11 in the cancer cell is a result of MRE11 gene amplification in the cancer cell. In some embodiments, the increased level and/or activity of MRE11 in the cancer cell is a result of one or more activating amino acid substitutions in a protein encoded by a MRE11 gene.

In some embodiments, the subject has been diagnosed or identified as having a cancer. In some embodiments, the cancer is selected from the group consisting of: renal clear cell carcinoma, uveal melanoma, tongue squamous cell carcinoma, breast cancer, and skin cancer.

In some embodiments, the methods further comprise administering a therapeutically effective amount of a STING antagonist or cGAS inhibitor to a subject identified as having an increased likelihood of being responsive to treatment with a STING antagonist or cGAS inhibitor.

In some embodiments of any of the methods described herein, the STING antagonist is an inhibitory nucleic acid (e.g., a short interfering RNA, an antisense nucleic acid, a cyclic dinucleotide, or a ribozyme). In some embodiments of any of the methods described herein, the STING antagonist or cGAS inhibitor is any of the compounds described herein, or a pharmaceutically acceptable salt, solvate, or co-crystal thereof.

Additional exemplary aspects that can be used or incorporated in these methods are described herein.

### Indications

In some embodiments, methods for treating a subject having condition, disease or disorder in which an increase in cGAS/STING signaling activity and/or a decrease in TREX1 level and/or activity contributes to the pathology and/or symptoms and/or progression of the condition, disease or disorder are provided, comprising administering to a subject an effective amount of a chemical entity described herein (e.g., a compound described generically or specifically herein or a pharmaceutically acceptable salt thereof or compositions containing the same). In some embodiments of any of the methods described herein, the subject can have, or be identified or diagnosed as having, any of the conditions, diseases, or disorders in which an increase in cGAS/STING signaling activity and/or a decrease in TREX1 level and/or activity contributes to the pathology and/or symptoms and/or progression of the condition, disease, or disorder. In some embodiments of any of the methods described herein, the subject can be suspected of having or present with one or more symptoms of any of the conditions, diseases, or disorders described herein.

In some embodiments, the condition, disease or disorder is a cancer (e.g., renal clear cell carcinoma, uveal melanoma, tongue squamous cell carcinoma, breast cancer, and skin cancer).

### Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the STING antagonist or cGAS inhibitor (e.g., any of the STING antagonists or cGAS inhibitors described herein or known in the art).

In certain embodiments, the second therapeutic agent or regimen is administered to the subject prior to contacting with or administering the STING antagonist or cGAS inhibitor (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the STING antagonist or cGAS inhibitor. By way of example, the second therapeutic agent or regimen and the STING antagonist or cGAS inhibitor are provided to the subject simultaneously in the same dosage form. As another example, the second therapeutic agent or regimen and the STING antagonist or cGAS inhibitor are provided to the subject concurrently in separate dosage forms.

In still other embodiments, the second therapeutic agent or regimen is administered to the subject after contacting with or administering the STING antagonist or cGAS inhibitor (e.g., about one hour after, or about 6 hours after, or about 12 hours after, or about 24 hours after, or about 48 hours after, or about 1 week after, or about 1 month after).

### Patient Selection

In some embodiments, the methods described herein include the step of identifying a subject (e.g., a patient) in need of treatment as having a cell (e.g., a cancer cell) having one or both of (i) decreased TREX1 level and/or activity, and (ii) increased cGAS/STING signaling pathway activity (e.g., an increase of between 1% and 1000%, or any of the subranges of this range described herein) (e.g., as compared to a reference level) or to a subject identified as having an elevated level of cGAMP in a serum or a tumor sample (e.g., an increase of between 1% and 1000%, or any of the subranges of this range described herein) (e.g., as compared to a reference level). In some embodiments, the methods described herein include the step of identifying a subject (e.g., a patient) in need of treatment as having a cell (e.g., a cancer cell) having decreased TREX1 level and/or activity. In some embodiments, the methods described herein include the step of identifying a subject (e.g., a patient) in need of treatment as having a cell (e.g., a cancer cell) having increased cGAS/STING signaling pathway activity. In some embodiments, the methods described herein include the step of identifying a subject (e.g., a patient) in need of treatment as having a cell (e.g., a cancer cell) having both (i) decreased TREX1 level and/or activity, and (ii) increased cGAS/STING signaling pathway activity (e.g., an increase of between 1% and 1000%, or any of the subranges of this range described herein) (e.g., as compared to a reference level) or to a subject identified as having an elevated level of cGAMP in a serum or a tumor sample (e.g., an increase of between 1% and 1000%, or any of the subranges of this range described herein) (e.g., as compared to a reference level).

In some embodiments, the subject is identified as having a cell (e.g. a cancer cell) having a decreased TREX1 level. In some embodiments, the identification of the subject as having a cell (e.g., a cancer cell) having a decreased TREX1 level comprises detecting a decreased level of TREX1 protein in the cell. In some embodiments, the TREX1 level is a level of TREX1 protein in the cell. In some embodiments, the TREX1 level is a level of TREX1 mRNA in the cell. In some embodiments, the identification of the subject as having a cell (e.g., a cancer cell) having a decreased TREX1 level comprises detecting a decreased level of TREX1 mRNA in the cell. In some embodiments, the decreased TREX1 level and/or activity is a result of gene loss in the cell. In some embodiments, the TREX1 gene loss is loss of one allele of the TREX1 gene. In some embodiments, the TREX1 gene loss is loss of both alleles of the TREX1 gene. In some embodiments, the identification of the subject as having a cell (e.g., a cancer cell) having decreased TREX1 level and/or activity comprises detecting TREX1 gene loss in the cell. In some embodiments, the decreased TREX1 level and/or activity is a result of one or more amino acid deletions in a protein encoded by a TREX1 gene in the cell. In some embodiments, the identification of the subject as having a cell (e.g., a cancer cell) having decreased TREX1 level and/or activity comprises detecting one or more amino acid deletions in a protein encoded by a TREX1 gene in the cell. In some embodiments, the decreased TREX1 level and/or activity is a result of one or more inactivating amino acid substitutions in a protein encoded by a TREX1 gene in the cell. In some embodiments, identification of the subject as having a cancer cell having decreased TREX1 expression and/or activity comprises detecting one or more inactivating amino acid substitutions in a protein encoded by a TREX1 gene in the cell.

In some embodiments, the methods described herein include the step of identifying a subject (e.g., a patient) in need of treatment as having a cell (e.g., a cancer cell) having one or both of (i) decreased TREX1 level and/or activity, and (ii) increased STING signaling pathway activity, e.g., by detecting a gain-of-function mutation (e.g., a BRCA1 protein having a E111Gfs*3 frameshift insertion, numbered according to SEQ ID NO: 15, a BRCA1 protein having a N1784Kfs*3 frameshift insertion numbered according to SEQ ID NO: 25, a SAMHD1 protein having a V133I amino acid substitution numbered according to SEQ ID NO: 27, a DNASE2 protein having R314W amino acid substitution numbered according to SEQ ID NO: 33, a BLM protein having a N515Mfs*16 frameshift deletion numbered according to SEQ ID NO: 37, a PARP1 protein having a S507Afs*17 frameshift deletion numbered according to SEQ ID NO: 43, a RPA1 mRNA splicing having a X12 splice mutation, or a RAD51 protein having R254* amino acid substitution numbered according to SEQ ID NO: 51), or a loss-of-function mutation (e.g., any of the exemplary loss-of-function mutations described herein).

In some embodiments, the methods described herein include the step of identifying a subject (e.g., a patient) in need of treatment as having a cell (e.g., a cancer cell) having one or both of (i) decreased TREX1 level and/or activity, and (ii) increased cGAS/STING signaling pathway activity (e.g., using any of the exemplary methods described herein).

Methods of Detecting the Level of cGAS/STING Signaling Pathway Activity and/or Expression

In some embodiments of any of the methods described herein, the cGAS/STING signaling pathway activity is the secretion of a type I IFN or a type III IFN. In some embodiments of any of the methods described herein, the cGAS/STING signaling pathway activity is the secretion of IFN-α. In some embodiments of any of the methods described herein, the cGAS/STING signaling pathway activity is the secretion of IFN-β. Non-limiting examples of methods that can be used to detect the secretion of IFN-α and IFN-β include immunohistochemistry, immunoassays, e.g., enzyme-linked immunosorbent assay (ELISA), sandwich ELISA, immunoprecipitation, and immunofluorescent assay.

Non-limiting methods of detecting cGAMP in serum or tissue include immunoassays, e.g., enzyme-linked immunosorbent assay (ELISA), sandwich ELISA, immunoprecipitation, and immunofluorescent assay) an mass spectrometry.

In some embodiments of any of the methods described herein, the cGAS/STING signaling pathway activity can be the level and/or activity of an upstream activator in the cGAS/STING signaling pathway (e.g., the level of one or more (e.g., two, three, four, five, or six) of MUS81 mRNA, MUS81 protein, IFI16 mRNA, IFI16 protein, cGAS mRNA, cGAS protein, DDX41 mRNA, DDX41 protein, EXO1 mRNA, EXO1 protein, DNA2 mRNA, DNA2 protein, RBBP8 mRNA, RBBP8 protein, MRE11 mRNA, or MRE11 protein in a mammalian cell (e.g., a mammalian cell obtained from a subject). In some embodiments of any of the methods described herein, the cGAS/STING signaling pathway activity can be determined by detecting the level and/or activity of an upstream suppressor of the cGAS/STING signaling pathway (e.g., the level of one or more (e.g., two, three, four, five, or six) of BRCA1 mRNA, BRCA1 protein, BRCA2 mRNA, BRCA2 protein, SAMHD1 mRNA, SAMHD1 protein, DNASE2 mRNA, DNASE2 protein, BLM mRNA, BLM protein, PARP1 mRNA, PARP1 protein, RPA1 mRNA, RPA1 protein, RAD51 mRNA, or RAD51 protein in a mammalian cell (e.g., a mammalian cell obtained from a subject).

Non-limiting assays that can be used to determine the level and/or activity of an upstream activator or upstream suppressor of the STING pathway include: Southern blot analysis, Norther blot analysis, polymerase chain reaction (PCR)-based methods, e.g., next generation sequencing, reverse transcription polymerase chain reaction (RT-PCR), TaqMan™, microarray analysis, immunohistochemistry, immunoassays, e.g., enzyme-linked immunosorbent assay (ELISA), sandwich ELISA, immunoprecipitation, immunofluorescent assay, mass spectrometry, immunoblot (Western blot), RIA, and flow cytometry.

In some embodiments of any of the methods described herein, a mammalian cell having an increased level of cGAS/STING signaling pathway activity can be identified by detecting the presence of one of more of the following the mammalian cell: a gain-of-function mutation in a cGAS/STING signaling pathway gene (e.g., a BRCA1 protein having a E111Gfs*3 frameshift insertion, numbered according to SEQ ID NO: 15, a BRCA1 protein having a N1784Kfs*3 frameshift insertion numbered according to SEQ ID NO: 25, a SAMHD1 protein having a V133I amino acid substitution numbered according to SEQ ID NO: 27, a DNASE2 protein having R314W amino acid substitution numbered according to SEQ ID NO: 33, a BLM protein having a N515Mfs*16 frameshift deletion numbered according to SEQ ID NO: 37, a PARP1 protein having a S507Afs*17 frameshift deletion numbered according to SEQ ID NO: 43, a RPA1 mRNA splicing having a X12 splice mutation, or a RAD51 protein having R254* amino acid substitution numbered according to SEQ ID NO: 51).

In some embodiments of any of the methods described herein, a mammalian cell having decreased level and/or activity of TREX1 can be identified by, e.g., detecting the presence of a loss-of-function mutation in a TREX1 gene (e.g., a TREX1 gene loss (e.g., loss of TREX1 in one or both alleles), an amino acid deletion in the protein encoded by a TREX1 bene, or an inactivating amino acid substitution in a protein encoded by a TREX1 gene). Non-limiting examples of assays that can be used to determine the level of the presence of any of these mutations (e.g., any of the mutations described herein) include Southern blot analysis, Northern blot analysis, mass spectrometry, UV absorbance, lab-on-a-chip, microfluidics, gene chip, intercalating dyes (e.g., ethidium bromide), gel electrophoresis, restriction digestion and electrophoresis, and sequencing (e.g., using any of the wide variety of sequencing methods described herein or known in the art), including polymerase chain reaction (PCR)-based methods, e.g., next generation sequencing, reverse transcription polymerase chain reaction (RT-PCR), TaqMan™, and microarray analysis.

For example, the detection of genomic DNA can include detection of the presence of one or more unique sequences found in genomic DNA (e.g., human genomic DNA) (e.g., satellite DNA sequences present in centromeres or heterochromatin, minisatellite sequences, microsatellite sequences, the sequence of a transposable element, a telomere sequence, a specific sequence (e.g., 250 base pairs to about 300 base pairs) containing one or more SNPs, or a specific sequence encoding a gene). Detection can be performed using labeled probes (e.g., fluorophore-, radioisotope-, enzyme-, quencher-, and enzyme-labeled probes), e.g., by hybridizing labeled probes to the genomic DNA present in the isolated genomic DNA sample or the control sample (e.g., in an electrophoretic gel) or hybridizing the labeled probes to the products of a PCR assay (e.g., a real-time PCR assay) or an assay that includes a PCR assay that utilized genomic DNA in the isolated genomic DNA test sample or the control sample as the template. Non-limiting examples of methods that can be used to generate probes include nick translation, random oligo primed synthesis, and end labeling.

A variety of assays for determining the genotype of a gene are known in the art. Non-limiting examples of such assays (which can be used in any of the methods described herein) include: dynamic allele-specific hybridization (see, e.g., Howell et al., Nature Biotechnol. 17:87-88, 1999), molecular beacon assays (see, e.g., Marras et al., “Genotyping Single Nucleotide Polymorphisms with Molecular Beacons,” In Kwok (Ed.), Single Nucleotide Polymorphisms: Methods and Protocols, Humana Press, Inc., Totowa, N.J., Vol. 212, pp. 111-128, 2003), microarrays (see, e.g., Affymetrix Human SNP 5.0 GeneChip), restriction fragment length polymorphism (RFLP) (see, e.g., Ota et al., Nature Protocols 2:2857-2864, 2007), PCR-based assays (e.g., tetraprimer ARMS-PCR (see, e.g., Zhang et al., Plos One 8:e62126, 2013), real-time PCR, allele-specific PCR (see, e.g., Gaudet et al., Methods Mol. Biol. 578:415-424, 2009), and TaqMan Assay SNP Genotyping (see, e.g., Woodward, Methods Mol. Biol. 1145:67-74, 2014, and TaqMan® OpenArray® Genotyping Plates from Life Technologies)), Flap endonuclease assays (also called Invader assays) (see, e.g., Olivier et al., Mutat. Res. 573:103-110, 2005), oligonucleotide ligation assays (see, e.g., Bruse et al., Biotechniques 45:559-571, 2008), single strand conformational polymorphism assays (see, e.g., Tahira et al., Human Mutat. 26:69-77, 2005), temperature gradient gel electrophoresis (see, e.g., Jones et al., “Temporal Temperature Gradient Electrophoresis for Detection of Single Nucleotide Polymorphisms,” in Single Nucleotide Polymophisms: Methods and Protocols, Volume 578, pp. 153-165, 2008) or temperature gradient capillary electrophoresis, denaturing high performance liquid chromatography (see, e.g., Yu et al., J. Clin. Pathol. 58:479-485, 2005), high-resolution melting of an amplified sequence containing the SNP (see, e.g., Wittwer et al., Clinical Chemistry 49:853-860, 2003), or sequencing (e.g., Maxam-Gilbert sequencing, chain-termination methods, shotgun sequencing, bridge PCR, and next-generation sequencing methods (e.g., massively parallel signature sequencing, polony sequencing, 454 pyrosequencing, Illumina (Solexa) sequencing, SOLiD sequencing, Ion Torrent semiconductor sequence, DNA nanoball sequencing, heliscope single molecule sequencing, and single molecule real-time sequencing). Additional details and a summary of various next-generation sequencing methods are described in Koboldt et al., Cell 155:27-38, 2013.

In some embodiments of any of the methods described herein, the genotyping of a gene includes a PCR assay (e.g., a real-time PCR-assay) (with or without a prior pre-amplification step (e.g., any of the pre-amplification methods described herein)). In some embodiments of any of the methods described herein the genotyping can be performed using TaqMan®-based sequencing (e.g., TaqMan®-based OpenArray® sequencing, e.g., high throughput TaqMan®-based Open Array® sequencing) (with or without a prior pre-amplification step (e.g., any of the pre-amplification methods described herein)).

In some embodiments of any of the methods described herein, the level of the protein or mRNA can be detected in a biological sample including blood, serum, exosomes, plasma, tissue, urine, feces, sputum, and cerebrospinal fluid.

In some embodiments of any of the methods described herein, the level of at least one (e.g., 2, 3, 4, 5, 6, 7 or 8) parameters related to cGAS/STING signaling pathway activity and/or expression can be determined, e.g., in any combination.

In one aspect, the cell can be a cell isolated from a subject who has been screened for the presence of a cancer or an indication that is associated with an increase in a cGAS/STING signaling pathway activity and/or a decrease in TREX1 level or activity.

### Reference Levels

In some embodiments of any of the methods described herein, the reference can be a corresponding level detected in a similar cell or sample obtained from a healthy subject (e.g., a subject that has not been diagnosed or identified as having a cancer, or any disorder associated with increased cGAS/STING signaling pathway activity and/or decreased TREX1 level and/or activity) (e.g., a subject who is not suspected or is not at increased risk of developing a cancer, or any disorder associated with increased cGAS/STING signaling pathway and/or decreased TREX1 level and/or activity and/or expression) (e.g., a subject that does not present with any symptom of a cancer, or any disorder associated with increased cGAS/STING signaling pathway activity and/or decreased TREX1 level and/or activity).

In some embodiments, a reference level can be a percentile value (e.g., mean value, 99% percentile, 95% percentile, 90% percentile, 85% percentile, 80% percentile, 75% percentile, 70% percentile, 65% percentile, 60% percentile, 55% percentile, or 50% percentile) of the corresponding levels detected in similar samples in a population of healthy subjects (e.g., a population of subjects that have not been diagnosed or identified as having a cancer, or any disorder associated with increased cGAS/STING signaling pathway and/or decreased TREX1 level and/or activity) (e.g., a population of subjects who are not suspected or are not at increased risk of developing a cancer, or any disorder associated with increased cGAS/STING signaling pathway and/or decreased TREX1 level and/or activity) (e.g., a population of subjects that do not present with any symptom of a cancer, or any disorder associated with increased cGAS/STING signaling pathway and/or decreased TREX1 level and/or activity).

In some embodiments, a reference can be a corresponding level detected in a similar sample obtained from the subject at an earlier time point.

### STING Antagonists

In any of the methods described herein, the STING antagonist can be any of the STING antagonists described herein (e.g., any of the compounds described in this section). In any of the methods described herein, the STING antagonist has an IC₅₀ of between about 1 nM and about 10 μM for STING.

In one aspect, the STING antagonist is a compound of Formula (I):

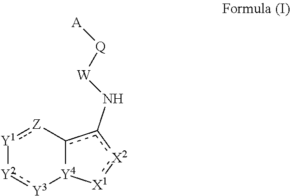

or a pharmaceutically acceptable salt thereof or an N-oxide thereof,
wherein:
Z is selected from the group consisting of a bond, CR¹, C(R³)₂, N, and NR²;
each of Y¹, Y², and Y³ is independently selected from the group consisting of O, S, CR¹, C(R³)₂, N, and NR²;

### Y⁴ is C or N;

X¹ is selected from the group consisting of O, S, N, NR², and CR¹;
X² is selected from the group consisting of O, S, N, NR⁴, and CR⁵;
each [symbol] is independently a single bond or a double bond, provided that the five-membered ring comprising Y⁴, X¹, and X² is heteroaryl;
W is selected from the group consisting of:
(i) C(═O); (ii) C(═S); (iii) S(O)₁₋₂; (iv) C(═NR^{d}); (v) C(═NH); (vi) C(═C—NO₂); (vii) S(O)N(R^{d}); and (viii) S(O)NH;
Q-A is defined according to (A) or (B) below:

### A

Q is NH or N(C₁₋₆ alkyl) wherein the C₁₋₆ alkyl is optionally substituted with 1-2 independently selected R^{a}, and

### A is:

(i) —(Y^{A1})ₙ—Y^{A2}, wherein:
n is 0 or 1;
Y^{A1} is C₁₋₆ alkylene, which is optionally substituted with from 1-6 R^{a}; and
Y^{A2} is:
   (a) C₃₋₂₀ cycloalkyl, which is optionally substituted with from 1-4 R^{b},
   (b) C₆₋₂₀ aryl, which is optionally substituted with from 1-4 R^{c};
   (c) heteroaryl including from 5-20 ring atoms, wherein from 1-4 ring atoms are heteroatoms, each independently selected from the group consisting of N, N(H), N(R^{d}), O, and S, and wherein one or more of the heteroaryl ring carbon atoms are optionally substituted with from 1-4 independently selected R^{c}, or
   (d) heterocyclyl including from 3-16 ring atoms, wherein from 1-3 ring atoms are heteroatoms, each independently selected from the group consisting of N, N(H), N(R^{b}), N(R^{d}), and O, and wherein one or more of the heterocyclyl ring carbon atoms are optionally substituted with from 1-4 independently selected R^{b},

### OR

(ii) —Z¹—Z²—Z³, wherein:
Z¹ is C₁₋₃ alkylene, which is optionally substituted with from 1-4 R^{a};
Z² is —N(H)—, —N(R^{d})—, —O—, or —S—; and
Z³ is C₂₋₇ alkyl, which is optionally substituted with from 1-4 R^{a};

### OR

(iii) C₁₋₁₀ alkyl, which is optionally substituted with from 1-6 independently selected R^{a}, or

### B

Q and A, taken together, form:

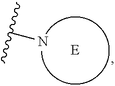

wherein [symbol] denotes point of attachment to W; and

E is a ring including from 3-16 ring atoms, wherein aside from the nitrogen atom present, from 0-3 additional ring atoms are heteroatoms, each independently selected from the group consisting of N, N(H), N(R^{d}), and O, and wherein one or more of the heterocyclyl ring carbon atoms are optionally substituted with from 1-4 independently selected R^{b},

each occurrence of R¹ is independently selected from the group consisting of H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —(C₀₋₃ alkylene)-C₃₋₆ cycloalkyl optionally substituted with from 1-4 independently selected R^{g}; —(C₀₋₃ alkylene)-C₆₋₁₀ aryl optionally substituted with from 1-4 independently selected R^{g}; —(C₀₋₃ alkylene)-5-10 membered heteroaryl, wherein from 1-3 ring atoms of the heteroaryl are heteroatoms each independently selected from the group consisting of N, NH, NR^{d}, O, and S, wherein the heteroaryl is optionally substituted with from 1-4 independently selected R^{g}; —(C₀₋₃ alkylene)-5-10 membered heterocyclyl, wherein from 1-3 ring atoms of the heterocyclyl are heteroatoms each independently selected from the group consisting of N, NH, NR^{d}, O, and S, wherein the heterocyclyl is optionally substituted with 1-4 independently selected R^{g}; —S(O)₁₋₂(C₁₋₄ alkyl); —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″);
each occurrence of R² is independently selected from the group consisting of:
(i) C₁₋₆ alkyl, which is optionally substituted with from 1-2 independently selected R^{a};
(ii) C₃₋₆ cycloalkyl;
(iii) heterocyclyl including from 3-10 ring atoms, wherein from 1-3 ring atoms are heteroatoms, each independently selected from the group consisting of N, N(H), N(R^{d}), and O;
(iv) —C(O)(C₁₋₄ alkyl);
(v) —C(O)O(C₁₋₄ alkyl);

### (vi) —CON(R′)(R″);

(vii) —S(O)₁₋₂(NR′R″);
(viii) —S(O)₁₋₂(C₁₋₄ alkyl);

### (ix) —OH;

(x) C₁₋₄ alkoxy; and

### (xi) H;

each occurrence of R³ is independently selected from the group consisting of H, C₁₋₆ alkyl optionally substituted with from 1-6 independently selected R^{a}; C₁₋₄ haloalkyl; —OH; —F; —Cl; —Br; —NR^{e}R^{f}; C₁₋₄ alkoxy; C₁₋₄ haloalkoxy; —C(═O)(C₁₋₄ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and C₃₋₆ cycloalkyl optionally substituted with from 1-4 independently selected C₁₋₄ alkyl; or two
R³ on the same carbon combine to form an oxo;
R⁴ is selected from the group consisting of H and C₁₋₆ alkyl;
R⁵ is selected from the group consisting of H, halo, C₁₋₄ alkoxy, OH, oxo, and C₁₋₆ alkyl;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;

each occurrence of R^{b} is independently selected from the group consisting of: C₁. alkyl optionally substituted with from 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₄ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; (C₀₋₃ alkylene)-C6-10 aryl optionally substituted with 1-4 independently selected C₁₋₄ alkyl; and (C₀₋₃ alkylene)-C₃₋₆ cycloalkyl optionally substituted with from 1-4 independently selected C₁₋₄ alkyl;

each occurrence of R^{c} is independently selected from the group consisting of:
(i) halo;
(ii) cyano;
(iii) C₁₋₁₀ alkyl which is optionally substituted with from 1-6 independently selected R^{a};
(iv) C₂₋₆ alkenyl;
(v) C₂₋₆ alkynyl;
(vi) C₁₋₄ haloalkyl;
(vii) C₁₋₄ alkoxy;
(viii) C₁₋₄ haloalkoxy;
(ix) —(C₀₋₃ alkylene)-C₃₋₆ cycloalkyl optionally substituted with from 1-4 independently selected C₁₋₄ alkyl;
(x) —(C₀₋₃ alkylene)-heterocyclyl, wherein the heterocyclyl includes from 3-16 ring atoms, wherein from 1-3 ring atoms are heteroatoms, each independently selected from the group consisting of N, N(H), N(R^{d}), and O;
(xi) —S(O)₁₋₂(C₁₋₄ alkyl);
(xii) —NR^{e}R^{f};
(xiii) —OH;
(xiv) —S(O)₁₋₂(NR′R″);
(xv) —C₁₋₄ thioalkoxy;
(xvi) —NO₂;
(xvii) —C(═O)(C₁₋₄ alkyl);
(xviii) —C(═O)O(C₁₋₄ alkyl);
(xix) —C(═O)OH;

### (xx) —C(═O)N(R′)(R″);

(xxi) —(C₀₋₃ alkylene)-C₆₋₁₀ aryl optionally substituted with from 1-4 independently selected C₁₋₄ alkyl; and
(xxii) —(C₀₋₃ alkylene)-5-10 membered heteroaryl, wherein from 1-3 ring atoms of the heteroaryl are heteroatoms each independently selected from the group consisting of N, NH, NR^{d}, O, and S, wherein the heteroaryl is optionally substituted with from 1-4 independently selected C₁₋₄ alkyl;

R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —CN; —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R^{e} and R^{f}), which are each independently selected from the group consisting of N(R^{d}), O, and S;

each occurrence of R^{g} is independently selected from the group consisting of: halo; cyano; C₁₋₆ alkyl optionally substituted with from 1-2 independently selected R^{a}; C₁₋₄ haloalkyl; C₁₋₆ alkoxy optionally substituted with 1-2 independently selected R^{a}; C₁₋₄ haloalkoxy; S(O)₁₋₂(C₁₋₄ alkyl); —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″); and

each occurrence of R′ and R″ is independently selected from the group consisting of: H and C₁₋₄ alkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of: H and C₁₋₃ alkyl; and (b) from 0-3 ring heteroatoms (in addition to the nitrogen atom attached to R′ and R″), which are each independently selected from the group consisting of N(R^{d}), O, and S,

provided that one or more of a), b), and c) apply:

a) one or more of Z, Y¹, Y², Y³, and Y⁴ in the ring below

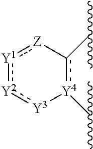

is an independently selected heteroatom;

b) the ring that includes Z, Y¹, Y², Y³, and Y⁴ is partially unsaturated; OR

c) Z is a bond;

further provided that when Q-A is defined according to (A); A is C₆ aryl mono-substituted with C₄ alkyl such as n-butyl at the para position; and the ring that includes Z, Y¹, Y², Y³, and Y⁴ is aromatic, then the ring that includes Z, Y¹, Y², Y³, and Y⁴ must be substituted with one or more R¹ that is other than hydrogen; and

and further provided with the proviso that the compound is not selected from the group consisting of:

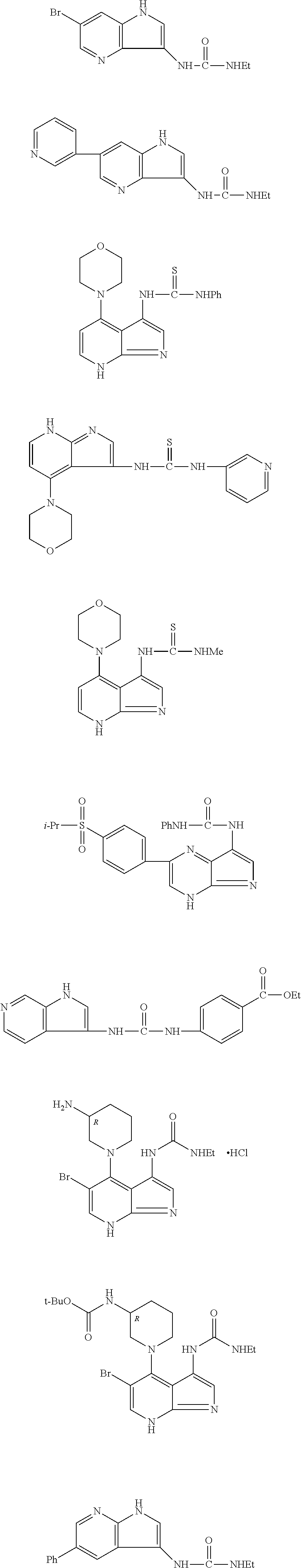

In some embodiments of the compound of Formula (I), Y⁴ is C; and/or X² is CR⁵ (e.g., CH); and/or X¹ is NR² (e.g., NH).

In some embodiments of the compound of Formula (I), wherein the ring that includes Z, Y¹, Y², Y³, and Y⁴:

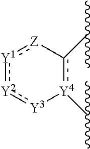

is aromatic. In certain of these embodiments, Z is other than a bond. In certain embodiments, from 1-2 (e.g., 1) of Z, Y¹, Y², Y³, and Y⁴ is independently N.

As a non-limiting example, the ring that includes Z, Y¹, Y², Y³, and Y⁴ can be selected from the group consisting of:

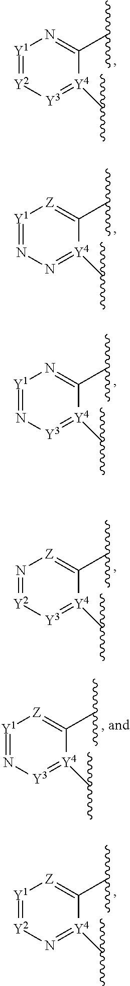

wherein each [symbol] denotes points of attachment to the ring comprising X¹ and X², and wherein the bottom [symbol] denotes point of attachment to X¹. For example,

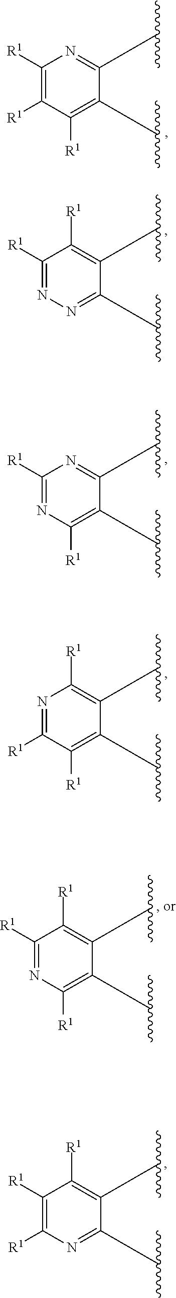

wherein each [symbol] denotes points of attachment to the ring comprising X¹ and X², and wherein the bottom [symbol] denotes point of attachment to X¹.

In some embodiments of the compound of Formula (I), Z is a bond. In some embodiments of the compound of Formula (I), the ring that includes Z, Y¹, Y², Y³, and Y⁴ is partially unsaturated.

In some embodiments of the compound of Formula (I), X¹ is NH.

In some embodiments of the compound of Formula (I), the compound of Formula (I) has a formula selected from the group consisting of:

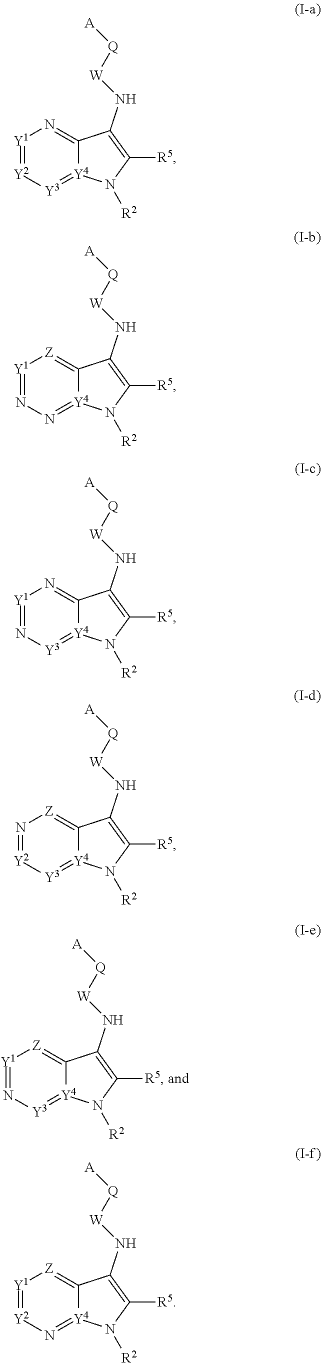

As a non-limiting example of the foregoing embodiments, the compound of Formula (I) can have formula selected from the group consisting of:

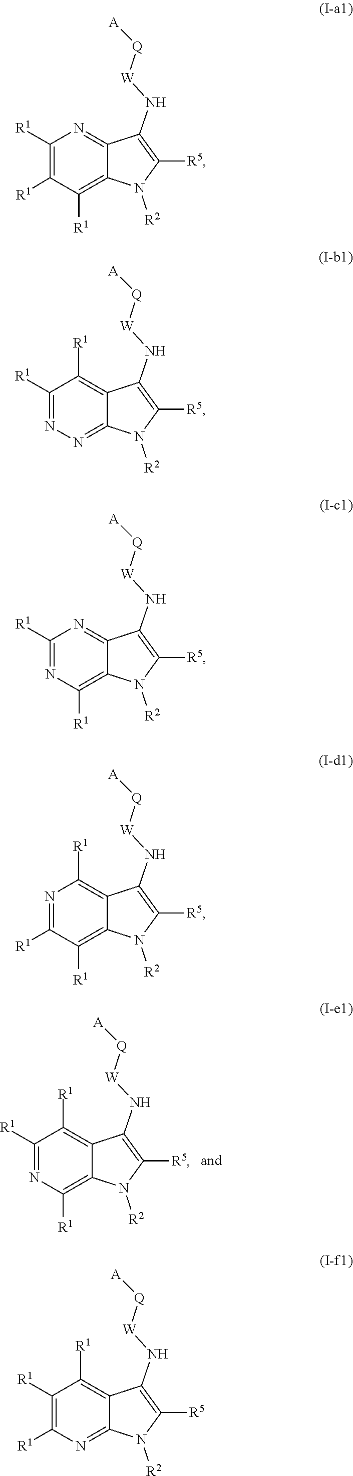

(e.g. in each of the foregoing formulae, R² can be H; and R⁵ can be H).

In some embodiments of the compound of Formula (I), W is C(═O).

In some embodiments of the compound of Formula (I), Q and A are defined according to (A). In some embodiments of the compound of Formula (I), A is —(Y^{A1})ₙ—Y^{A2}. In certain of these embodiments, n is 0. In certain other embodiments, n is 1. In certain of these embodiments, Y^{A1} is C₁₋₃ alkylene, such as CH₂ or CH₂CH₂.

In some embodiments, Y^{A} is C₆₋₂₀ aryl, which is optionally substituted with from 1-4 R^{e}. In some embodiments, Y^{A2} is heteroaryl including from 5-20 ring atoms, wherein from 1-4 ring atoms are heteroatoms, each independently selected from the group consisting of N, N(H), N(R^{d}), O, and S, and wherein one or more of the heteroaryl ring carbon atoms are optionally substituted with from 1-4 independently selected R^{e}. In some embodiments, Y^{A2} is C₃₋₂₀ cycloalkyl, which is optionally substituted with from 1-4 R^{b}. In some embodiments, Y^{A2} is heterocyclyl including from 3-12 ring atoms, wherein from 1-3 ring atoms are heteroatoms, each independently selected from the group consisting of N, N(H), N(R^{d}), and O, and wherein one or more of the heterocyclyl ring carbon atoms are optionally substituted with from 1-4 independently selected R^{b}.

In some embodiments of the compound of Formula (I), Q and A are defined according to (B).

In some embodiments, the STING antagonist is a compound of Formula (I):

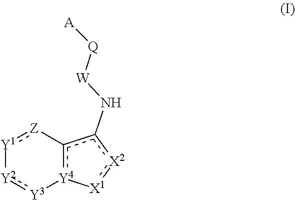

or a pharmaceutically acceptable salt thereof, or an N-oxide thereof,
wherein:
one or more of Z, Y¹, Y², Y³, and Y⁴ in the ring below

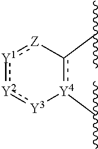

is an independently selected heteroatom;
Z is selected from the group consisting of CR¹ and N;
each of Y¹, Y², and Y³ is independently selected from the group consisting of CR¹ and N;
provided that one or more of Z, Y¹, Y², and Y³ is an independently selected CR¹;
Y⁴ is C; X¹ is NH; X² is CH;
each [symbol] is independently a single bond or a double bond, provided that the five-membered ring comprising Y⁴, X¹, and X² is heteroaryl; and the ring that includes Z, Y¹, Y², Y³, and Y⁴ is aromatic;

W is selected from the group consisting of: (i) C(═O); (ii) C(═S); (iv) C(═NR^{d}); and (v) C(═NH);

Q-A is defined according to (A) or (B) below:

### A

Q is NH or N(C₁₋₆ alkyl) wherein the C₁₋₆ alkyl is optionally substituted with 1-2 independently selected R^{a}, and

### A is:

(i) —(Y^{A1})ₙ—Y^{A2}, wherein:
n is 0 or 1;
Y^{A1} is C₁₋₆ alkylene, which is optionally substituted with from 1-6 R^{a}; and
Y^{A2} is:
   (a) C₃₋₂₀ cycloalkyl, which is optionally substituted with from 1-4 R^{b},
   (b) C₆₋₂₀ aryl, which is optionally substituted with from 1-4 R^{c};
   (c) heteroaryl including from 5-20 ring atoms, wherein from 1-4 ring atoms are heteroatoms, each independently selected from the group consisting of N, N(H), N(R^{d}), O, and S, and wherein one or more of the heteroaryl ring carbon atoms are optionally substituted with from 1-4 independently selected R^{c}, or
   (d) heterocyclyl including from 3-16 ring atoms, wherein from 1-3 ring atoms are heteroatoms, each independently selected from the group consisting of N, N(H), N(R^{b}), N(R^{d}), and O, and wherein one or more of the heterocyclyl ring carbon atoms are optionally substituted with from 1-4 independently selected R^{b},

### OR

(ii) —Z¹—Z²—Z³, wherein:
Z¹ is C₁₋₃ alkylene, which is optionally substituted with from 1-4 R^{a};
Z² is —N(H)—, —N(R^{d})—, —O—, or —S—; and
Z³ is C₂₋₇ alkyl, which is optionally substituted with from 1-4 R^{a};

### OR

(iii) C₁₋₁₀ alkyl, which is optionally substituted with from 1-6 independently selected R^{a},

### OR

### B

Q and A, taken together, form:

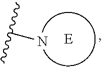

wherein [symbol] denotes point of attachment to W; and
E is a ring including from 3-16 ring atoms, wherein aside from the nitrogen atom present, from 0-3 additional ring atoms are heteroatoms, each independently selected from the group consisting of N, N(H), N(R^{d}), and O, and wherein one or more of the heterocyclyl ring carbon atoms are optionally substituted with from 1-4 independently selected R^{b}, each occurrence of R¹ is independently selected from the group consisting of H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —(C₀₋₃ alkylene)-C₃₋₆ cycloalkyl optionally substituted with from 1-4 independently selected R^{g}; —(C₀₋₃ alkylene)-C₆₋₁₀ aryl optionally substituted with from 1-4 independently selected R^{g}; —(C₀₋₃ alkylene)-5-10 membered heteroaryl, wherein from 1-3 ring atoms of the heteroaryl are heteroatoms each independently selected from the group consisting of N, NH, NR^{d}, O, and S, wherein the heteroaryl is optionally substituted with from 1-4 independently selected R^{g}; —(C₀₋₃ alkylene)-5-10 membered heterocyclyl, wherein from 1-3 ring atoms of the heterocyclyl are heteroatoms each independently selected from the group consisting of N, NH, NR^{d}, O, and S, wherein the heterocyclyl is optionally substituted with 1-4 independently selected R^{g}; —S(O)₁₋₂(C₁₋₄ alkyl); —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″);

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl optionally substituted with from 1-4 independently selected C₁₋₄ alkyl;

each occurrence of R^{b} is independently selected from the group consisting of: C₁₋₁₀ alkyl optionally substituted with from 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₄ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; (C₀₋₃ alkylene)-C₆₋₁₀ aryl optionally substituted with 1-4 independently selected C₁₋₄ alkyl; and (C₀₋₃ alkylene)-C₃₋₆ cycloalkyl optionally substituted with from 1-4 independently selected C₁₋₄ alkyl;

each occurrence of R^{c} is independently selected from the group consisting of:
(i) halo;
(ii) cyano;
(iii) C₁₋₁₀ alkyl which is optionally substituted with from 1-6 independently selected R^{a};
(iv) C₂₋₆ alkenyl;
(v) C₂₋₆ alkynyl;
(vi) C₁₋₄ haloalkyl;
(vii) C₁₋₄ alkoxy;
(viii) C₁₋₄ haloalkoxy;
(ix) —(C₀₋₃ alkylene)-C₃₋₆ cycloalkyl optionally substituted with from 1-4 independently selected C₁₋₄ alkyl;
(x) —(C₀₋₃ alkylene)-heterocyclyl, wherein the heterocyclyl includes from 3-16 ring atoms, wherein from 1-3 ring atoms are heteroatoms, each independently selected from the group consisting of N, N(H), N(R^{d}), and O;
(xi) —S(O)₁₋₂(C₁₋₄ alkyl);
(xii) —NR^{e}R^{f};
(xiii) —OH;
(xiv) —S(O)₁₋₂(NR′R″);
(xv) —C₁₋₄ thioalkoxy;
(xvi) —NO₂;
(xvii) —C(═O)(C₁₋₄ alkyl);
(xviii) —C(═O)O(C₁₋₄ alkyl);
(xix) —C(═O)OH;

### (xx) —C(═O)N(R′)(R″); and

(xxi) —(C₀₋₃ alkylene)-C₆₋₁₀ aryl optionally substituted with from 1-4 independently selected C₁₋₄ alkyl; and
(xxii) —(C₀₋₃ alkylene)-5-10 membered heteroaryl, wherein from 1-3 ring atoms of the heteroaryl are heteroatoms each independently selected from the group consisting of N, NH, NR^{d}, O, and S, wherein the heteroaryl is optionally substituted with from 1-4 independently selected C₁₋₄ alkyl;

R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; —CN; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R^{e} and R^{f}), which are each independently selected from the group consisting of N(R^{d}), O, and S;

each occurrence of R^{g} is independently selected from the group consisting of: halo; cyano; C₁₋₆ alkyl optionally substituted with from 1-2 independently selected R^{a}; C₁₋₄ haloalkyl; C₁₋₆ alkoxy optionally substituted with 1-2 independently selected R^{a}; C₁₋₄ haloalkoxy; S(O)₁₋₂(C₁₋₄ alkyl); —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″); and

each occurrence of R′ and R″ is independently selected from the group consisting of: H and C₁₋₄ alkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R′ and R″), which are each independently selected from the group consisting of N(R^{d}), O, and S;

provided that when Q-A is defined according to (A); A is C₆ aryl mono-substituted with a C₄ alkyl such as n-butyl at the para position, then the ring that includes Z, Y¹, Y², Y³, and Y⁴ must be substituted with one or more R¹ that is other than hydrogen; and

further provided with the proviso that the compound is other than one or more of the following:

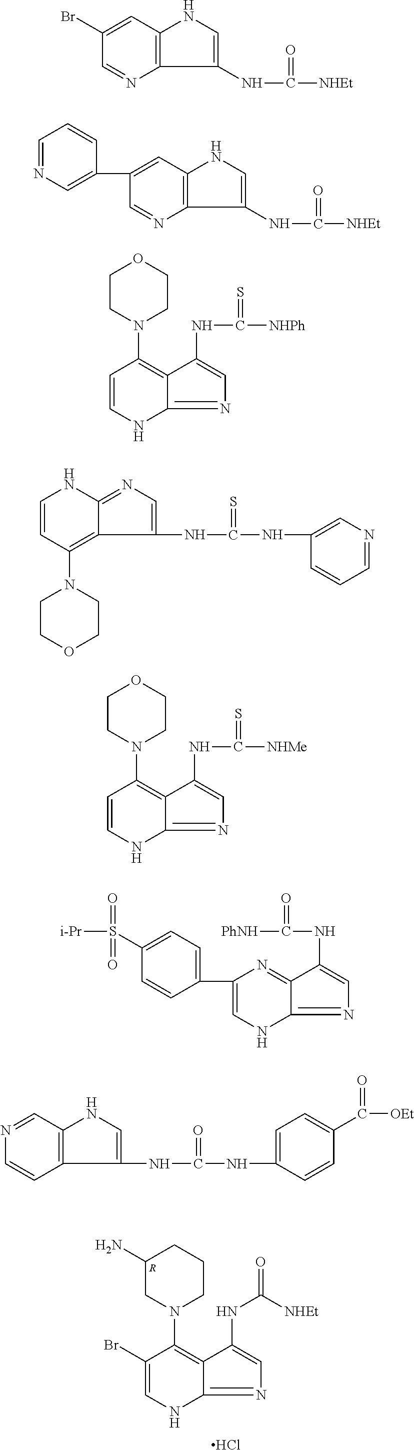

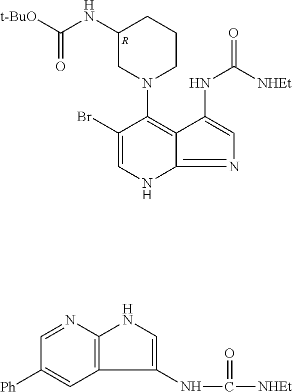

In another aspect, the STING antagonist is a compound selected from the group consisting of compounds in Table 1 and pharmaceutically acceptable salts thereof.

TABLE 1

| Compound | |
|---|---|
| # | Structure |
| 100 | 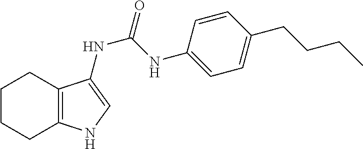 [SMILES: CCCCc1ccc(NC(=O)Nc2c[nH]c3c2CCCC3)cc1] |
| 102 | 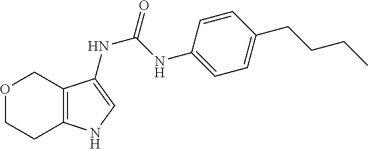 [SMILES: CCCCc1ccc(NC(=O)Nc2c[nH]c3c2COCC3)cc1] |
| 103 | 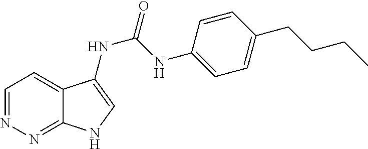 [SMILES: CCCCc1ccc(NC(=O)Nc2c[nH]c3nnccc23)cc1] |
| 104 | 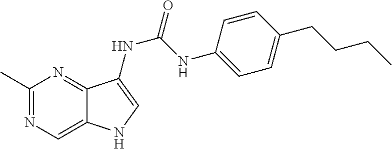 [SMILES: CCCCc1ccc(NC(=O)Nc2c[nH]c3cnc(C)nc23)cc1] |
| 105 | 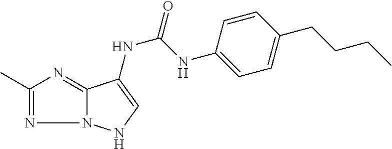 [SMILES: CCCCc1ccc(NC(=O)Nc2c[nH]n3nc(C)nc23)cc1] |
| 106 | 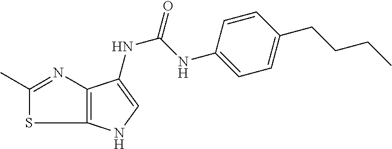 [SMILES: CCCCc1ccc(NC(=O)Nc2c[nH]c3sc(C)nc23)cc1] |
| 107 | 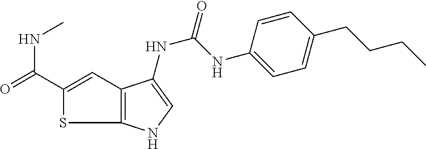 [SMILES: CCCCc1ccc(NC(=O)Nc2c[nH]c3sc(C(=O)NC)cc23)cc1] |
| 108 | 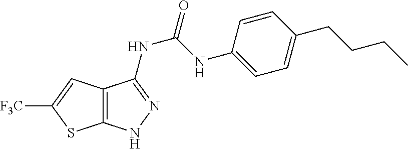 [SMILES: CCCCc1ccc(NC(=O)Nc2n[nH]c3sc(C(F)(F)F)cc23)cc1] |
| 109 | 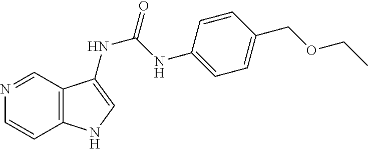 [SMILES: CCOCc1ccc(NC(=O)Nc2c[nH]c3ccncc23)cc1] |
| 110 | 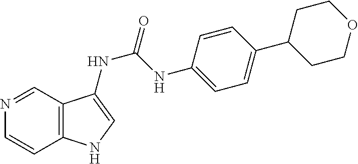 [SMILES: O=C(Nc1ccc(C2CCOCC2)cc1)Nc1c[nH]c2ccncc12] |
| 111 | 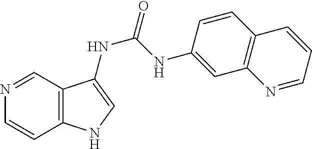 [SMILES: O=C(Nc1ccc2cccnc2c1)Nc1c[nH]c2ccncc12] |
| 112 | 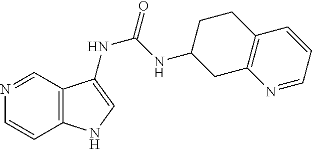 [SMILES: O=C(Nc1c[nH]c2ccncc12)NC1CCc2cccnc2C1] |
| 113 | 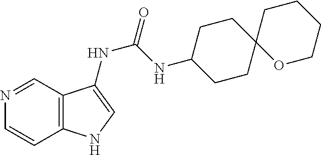 [SMILES: O=C(Nc1c[nH]c2ccncc12)NC1CCC2(CCCCO2)CC1] |
| 114 | 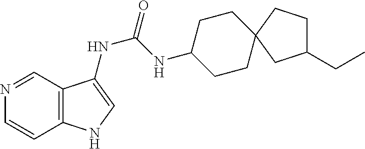 [SMILES: CCC1CCC2(CCC(NC(=O)Nc3c[nH]c4ccncc34)CC2)C1] |
| 115 | 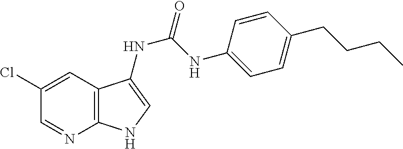 [SMILES: CCCCc1ccc(NC(=O)Nc2c[nH]c3ncc(Cl)cc23)cc1] |
| 116 | 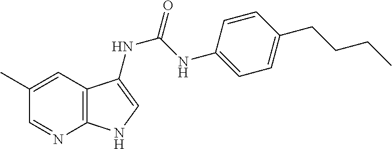 [SMILES: CCCCc1ccc(NC(=O)Nc2c[nH]c3ncc(C)cc23)cc1] |
| 117 | 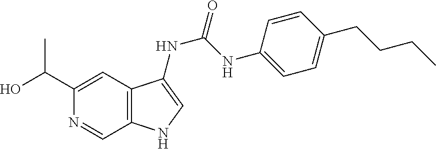 [SMILES: CCCCc1ccc(NC(=O)Nc2c[nH]c3cnc(C(C)O)cc23)cc1] |
| 118 | 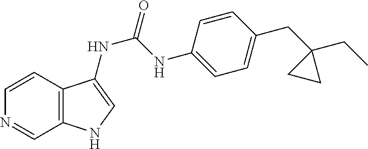 [SMILES: CCC1(Cc2ccc(NC(=O)Nc3c[nH]c4cnccc34)cc2)CC1] |
| 119 | 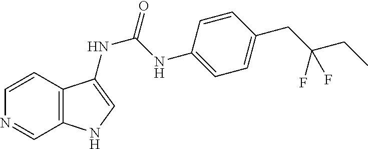 [SMILES: CCC(F)(F)Cc1ccc(NC(=O)Nc2c[nH]c3cnccc23)cc1] |
| 120 | 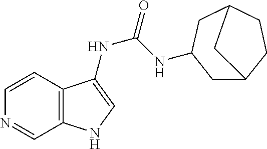 [SMILES: O=C(Nc1c[nH]c2cnccc12)NC1CC2CCC(C2)C1] |
| 121 | 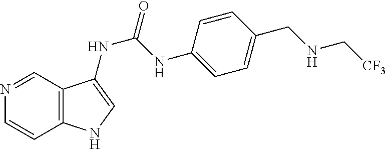 [SMILES: O=C(Nc1ccc(CNCC(F)(F)F)cc1)Nc1c[nH]c2ccncc12] |
| 122 | 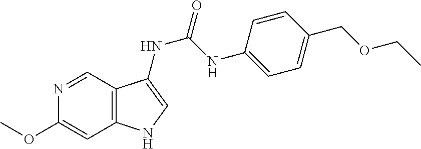 [SMILES: CCOCc1ccc(NC(=O)Nc2c[nH]c3cc(OC)ncc23)cc1] |
| 123 | 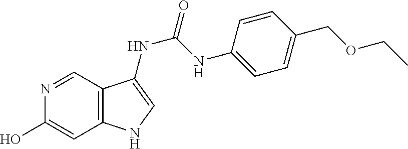 [SMILES: CCOCc1ccc(NC(=O)Nc2c[nH]c3cc(O)ncc23)cc1] |
| 124 | 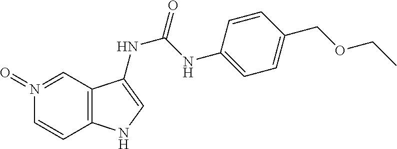 [SMILES: CCOCc1ccc(NC(=O)Nc2c[nH]c3cc[n+]([O-])cc23)cc1] |
| 125 | 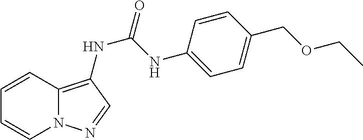 [SMILES: CCOCc1ccc(NC(=O)Nc2cnn3ccccc23)cc1] |
| 126 | 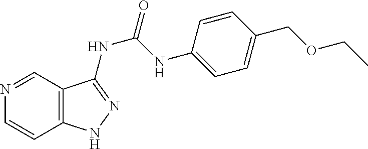 [SMILES: CCOCc1ccc(NC(=O)Nc2n[nH]c3ccncc23)cc1] |
| 127 | 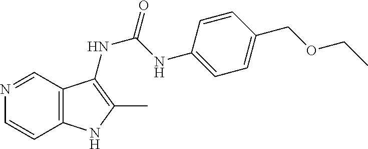 [SMILES: CCOCc1ccc(NC(=O)Nc2c(C)[nH]c3ccncc23)cc1] |
| 129 | 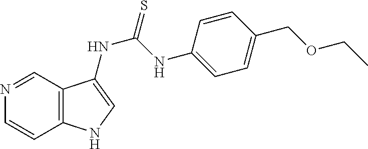 [SMILES: CCOCc1ccc(NC(=S)Nc2c[nH]c3ccncc23)cc1] |
| 130 | 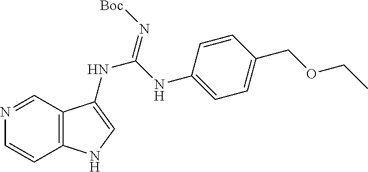 [SMILES: CCOCc1ccc(N/C(=N/C(=O)OC(C)(C)C)Nc2c[nH]c3ccncc23)cc1] |
| 131 | 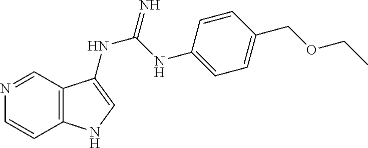 [SMILES: CCOCc1ccc(NC(=N)Nc2c[nH]c3ccncc23)cc1] |
| 132 | 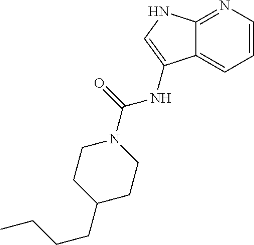 [SMILES: CCCCC1CCN(C(=O)Nc2c[nH]c3ncccc23)CC1] |
| 133 | 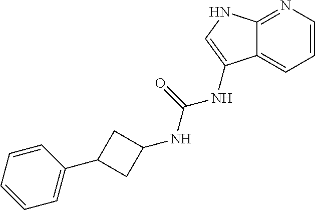 [SMILES: O=C(Nc1c[nH]c2ncccc12)NC1CC(c2ccccc2)C1] |
| 134 | 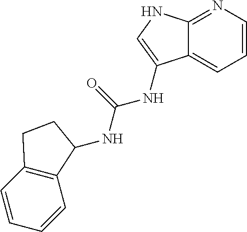 [SMILES: O=C(Nc1c[nH]c2ncccc12)NC1CCc2ccccc21] |
| 135 | 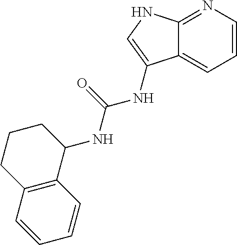 [SMILES: O=C(Nc1c[nH]c2ncccc12)NC1CCCc2ccccc21] |
| 136 | 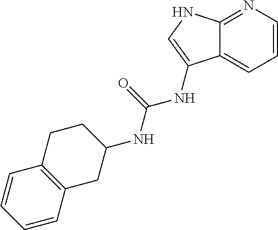 [SMILES: O=C(Nc1c[nH]c2ncccc12)NC1CCc2ccccc2C1] |
| 137 | 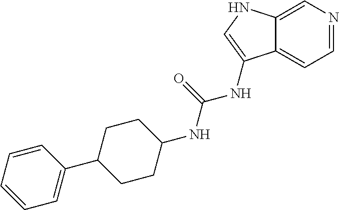 [SMILES: O=C(Nc1c[nH]c2cnccc12)NC1CCC(c2ccccc2)CC1] |
| 138 | 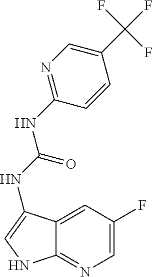 [SMILES: O=C(Nc1ccc(C(F)(F)F)cn1)Nc1c[nH]c2ncc(F)cc12] |
| 139 | 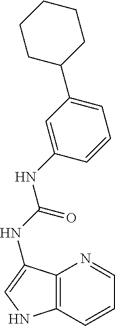 [SMILES: O=C(Nc1cccc(C2CCCCC2)c1)Nc1c[nH]c2cccnc12] |
| 140 | 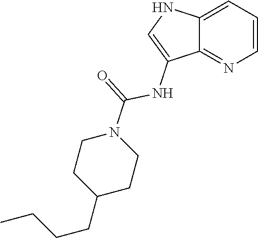 [SMILES: CCCCC1CCN(C(=O)Nc2c[nH]c3cccnc23)CC1] |
| 141 | 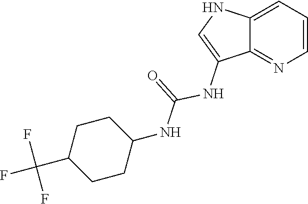 [SMILES: O=C(Nc1c[nH]c2cccnc12)NC1CCC(C(F)(F)F)CC1] |
| 142 | 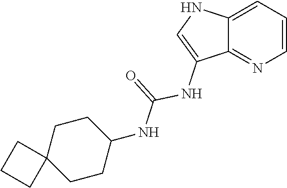 [SMILES: O=C(Nc1c[nH]c2cccnc12)NC1CCC2(CCC2)CC1] |
| 143 | 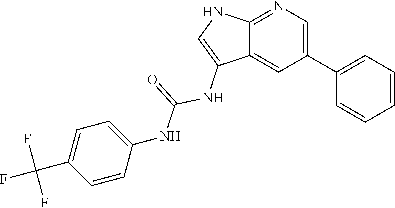 [SMILES: O=C(Nc1ccc(C(F)(F)F)cc1)Nc1c[nH]c2ncc(-c3ccccc3)cc12] |
| 144 | 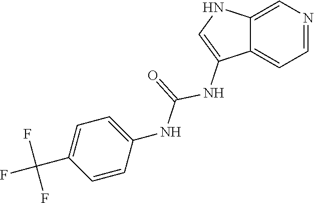 [SMILES: O=C(Nc1ccc(C(F)(F)F)cc1)Nc1c[nH]c2cnccc12] |
| 145 | 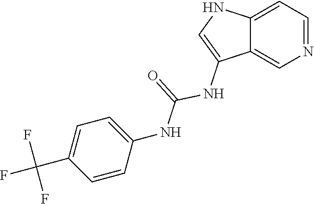 [SMILES: O=C(Nc1ccc(C(F)(F)F)cc1)Nc1c[nH]c2ccncc12] |
| 146 | 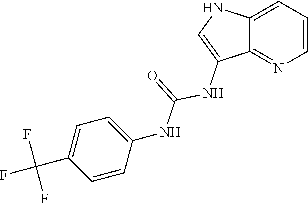 [SMILES: O=C(Nc1ccc(C(F)(F)F)cc1)Nc1c[nH]c2cccnc12] |
| 147 | 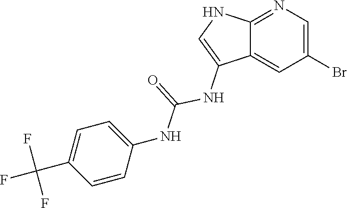 [SMILES: O=C(Nc1ccc(C(F)(F)F)cc1)Nc1c[nH]c2ncc(Br)cc12] |
| 148 | 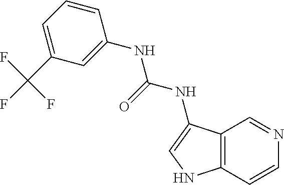 [SMILES: O=C(Nc1cccc(C(F)(F)F)c1)Nc1c[nH]c2ccncc12] |
| 149 | 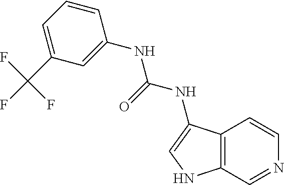 [SMILES: O=C(Nc1cccc(C(F)(F)F)c1)Nc1c[nH]c2cnccc12] |
| 150 | 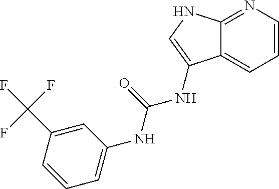 [SMILES: O=C(Nc1cccc(C(F)(F)F)c1)Nc1c[nH]c2ncccc12] |
| 151 | 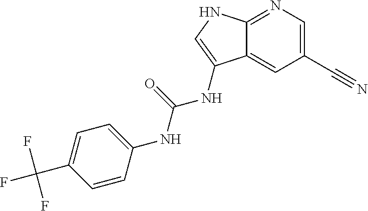 [SMILES: N#Cc1cnc2[nH]cc(NC(=O)Nc3ccc(C(F)(F)F)cc3)c2c1] |
| 152 | 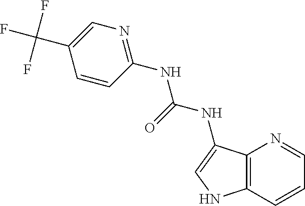 [SMILES: O=C(Nc1ccc(C(F)(F)F)cn1)Nc1c[nH]c2cccnc12] |
| 153 | 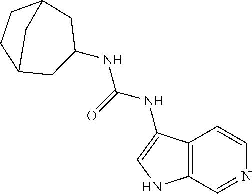 [SMILES: O=C(Nc1c[nH]c2cnccc12)NC1CC2CCC(C2)C1] |
| 154 | 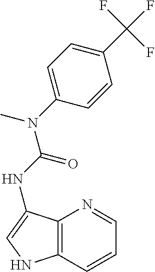 [SMILES: CN(C(=O)Nc1c[nH]c2cccnc12)c1ccc(C(F)(F)F)cc1] |
| 155 | 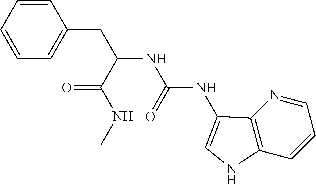 [SMILES: CNC(=O)C(Cc1ccccc1)NC(=O)Nc1c[nH]c2cccnc12] |
| 156 | 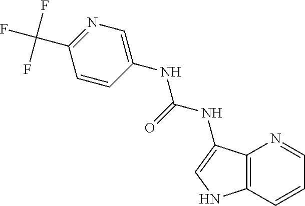 [SMILES: O=C(Nc1ccc(C(F)(F)F)nc1)Nc1c[nH]c2cccnc12] |
| 157 | 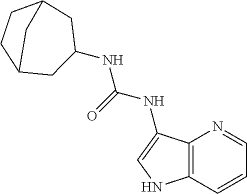 [SMILES: O=C(Nc1c[nH]c2cccnc12)NC1CC2CCC(C2)C1] |
| 158 | 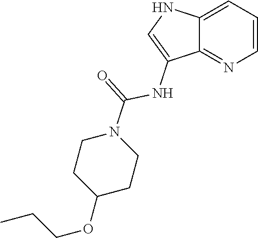 [SMILES: CCCOC1CCN(C(=O)Nc2c[nH]c3cccnc23)CC1] |
| 159 | 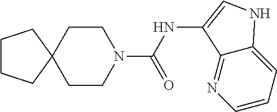 [SMILES: O=C(Nc1c[nH]c2cccnc12)N1CCC2(CCCC2)CC1] |
| 160 | 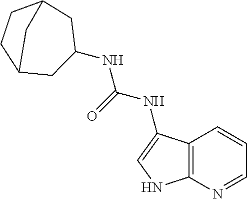 [SMILES: O=C(Nc1c[nH]c2ncccc12)NC1CC2CCC(C2)C1] |
| 161 | 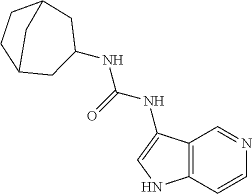 [SMILES: O=C(Nc1c[nH]c2ccncc12)NC1CC2CCC(C2)C1] |
| 162 | 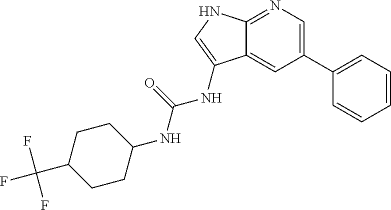 [SMILES: O=C(Nc1c[nH]c2ncc(-c3ccccc3)cc12)NC1CCC(C(F)(F)F)CC1] |
| 163 | 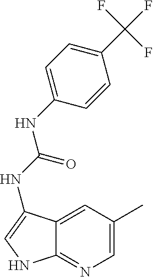 [SMILES: Cc1cnc2[nH]cc(NC(=O)Nc3ccc(C(F)(F)F)cc3)c2c1] |
| 164 | 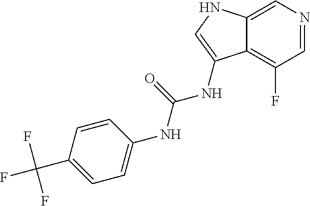 [SMILES: O=C(Nc1ccc(C(F)(F)F)cc1)Nc1c[nH]c2cncc(F)c12] |
| 165 | 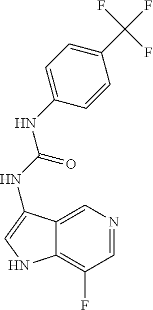 [SMILES: O=C(Nc1ccc(C(F)(F)F)cc1)Nc1c[nH]c2c(F)cncc12] |
| 166 | 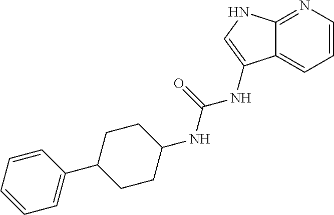 [SMILES: O=C(Nc1c[nH]c2ncccc12)NC1CCC(c2ccccc2)CC1] |
| 167 | 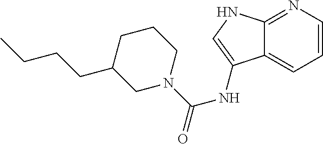 [SMILES: CCCCC1CCCN(C(=O)Nc2c[nH]c3ncccc23)C1] |
| 168 | 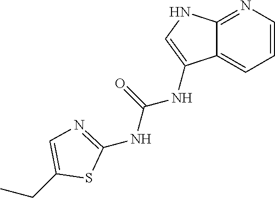 [SMILES: CCc1cnc(NC(=O)Nc2c[nH]c3ncccc23)s1] |
| 169 | 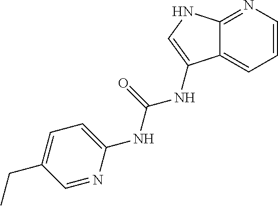 [SMILES: CCc1ccc(NC(=O)Nc2c[nH]c3ncccc23)nc1] |
| 170 | 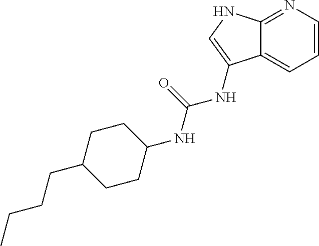 [SMILES: CCCCC1CCC(NC(=O)Nc2c[nH]c3ncccc23)CC1] |
| 171 | 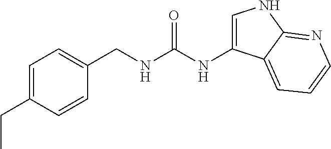 [SMILES: CCc1ccc(CNC(=O)Nc2c[nH]c3ncccc23)cc1] |
| 172 | 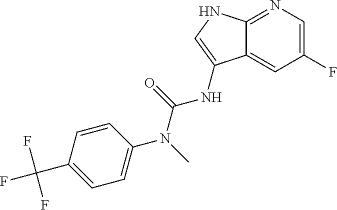 [SMILES: CN(C(=O)Nc1c[nH]c2ncc(F)cc12)c1ccc(C(F)(F)F)cc1] |
| 173 | 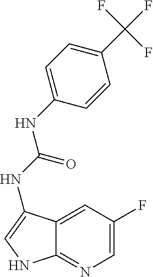 [SMILES: O=C(Nc1ccc(C(F)(F)F)cc1)Nc1c[nH]c2ncc(F)cc12] |
| 174 | 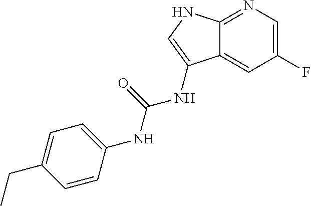 [SMILES: CCc1ccc(NC(=O)Nc2c[nH]c3ncc(F)cc23)cc1] |
| 179 | 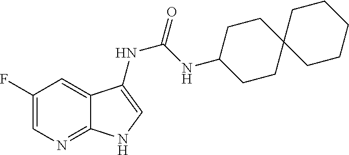 [SMILES: O=C(Nc1c[nH]c2ncc(F)cc12)NC1CCC2(CCCCC2)CC1] |
| 180 | 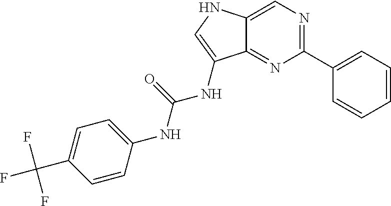 [SMILES: O=C(Nc1ccc(C(F)(F)F)cc1)Nc1c[nH]c2cnc(-c3ccccc3)nc12] |
| 181 | 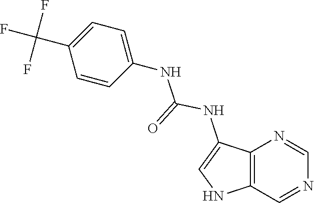 [SMILES: O=C(Nc1ccc(C(F)(F)F)cc1)Nc1c[nH]c2cncnc12] |
| 182 | 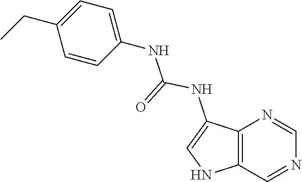 [SMILES: CCc1ccc(NC(=O)Nc2c[nH]c3cncnc23)cc1] |
| 183 | 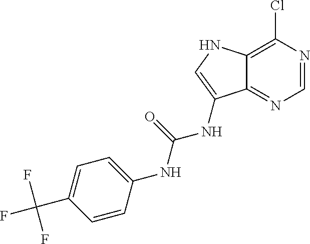 [SMILES: O=C(Nc1ccc(C(F)(F)F)cc1)Nc1c[nH]c2c(Cl)ncnc12] |
| 183b | 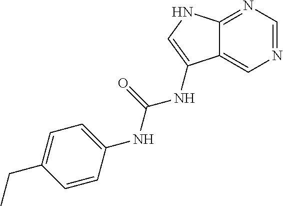 [SMILES: CCc1ccc(NC(=O)Nc2c[nH]c3ncncc23)cc1] |
| 184 | 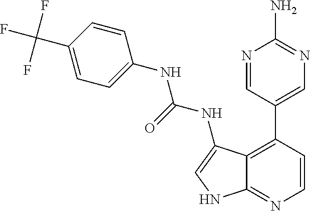 [SMILES: Nc1ncc(-c2ccnc3[nH]cc(NC(=O)Nc4ccc(C(F)(F)F)cc4)c23)cn1] |
| 185 | 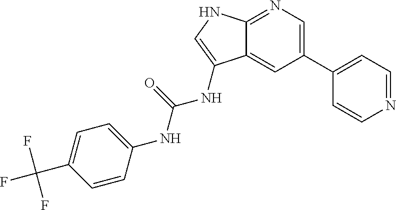 [SMILES: O=C(Nc1ccc(C(F)(F)F)cc1)Nc1c[nH]c2ncc(-c3ccncc3)cc12] |
| 186 | 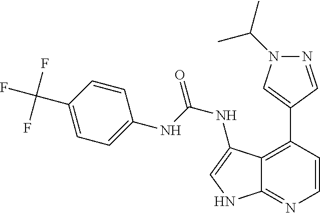 [SMILES: CC(C)n1cc(-c2ccnc3[nH]cc(NC(=O)Nc4ccc(C(F)(F)F)cc4)c23)cn1] |
| 187 | 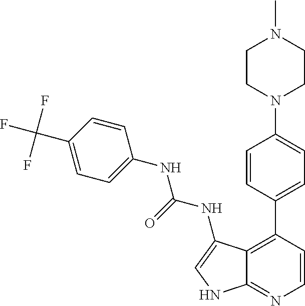 [SMILES: CN1CCN(c2ccc(-c3ccnc4[nH]cc(NC(=O)Nc5ccc(C(F)(F)F)cc5)c34)cc2)CC1] |
| 188 | 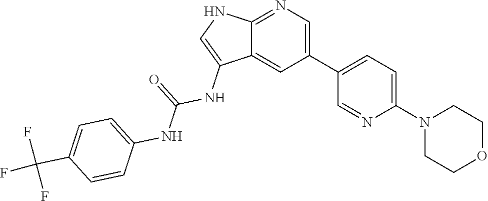 [SMILES: O=C(Nc1ccc(C(F)(F)F)cc1)Nc1c[nH]c2ncc(-c3ccc(N4CCOCC4)nc3)cc12] |
| 189 | 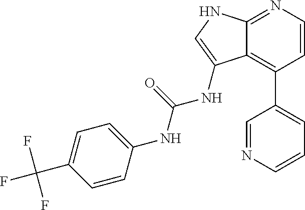 [SMILES: O=C(Nc1ccc(C(F)(F)F)cc1)Nc1c[nH]c2nccc(-c3cccnc3)c12] |
| 190 | 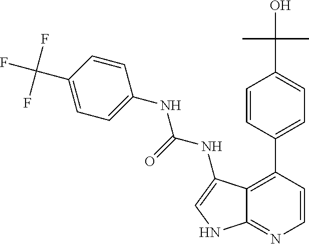 [SMILES: CC(C)(O)c1ccc(-c2ccnc3[nH]cc(NC(=O)Nc4ccc(C(F)(F)F)cc4)c23)cc1] |
| 191 | 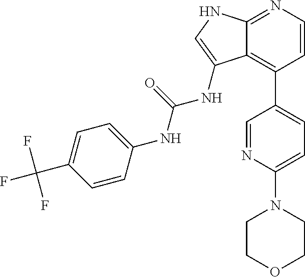 [SMILES: O=C(Nc1ccc(C(F)(F)F)cc1)Nc1c[nH]c2nccc(-c3ccc(N4CCOCC4)nc3)c12] |
| 192 | 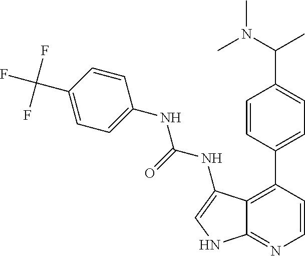 [SMILES: CC(c1ccc(-c2ccnc3[nH]cc(NC(=O)Nc4ccc(C(F)(F)F)cc4)c23)cc1)N(C)C] |
| 193 | 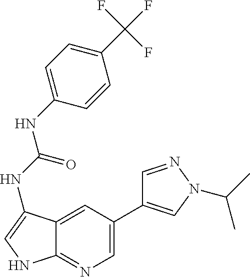 [SMILES: CC(C)n1cc(-c2cnc3[nH]cc(NC(=O)Nc4ccc(C(F)(F)F)cc4)c3c2)cn1] |
| 194 | 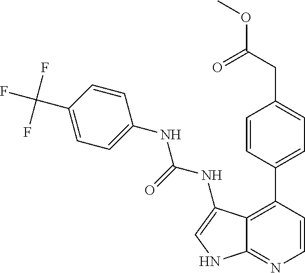 [SMILES: COC(=O)Cc1ccc(-c2ccnc3[nH]cc(NC(=O)Nc4ccc(C(F)(F)F)cc4)c23)cc1] |
| 195 | 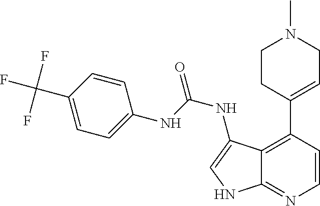 [SMILES: CN1CC=C(c2ccnc3[nH]cc(NC(=O)Nc4ccc(C(F)(F)F)cc4)c23)CC1] |
| 196 | 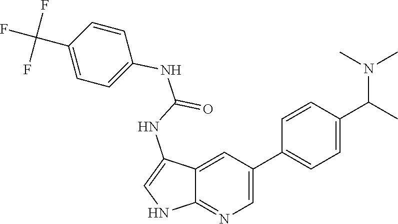 [SMILES: CC(c1ccc(-c2cnc3[nH]cc(NC(=O)Nc4ccc(C(F)(F)F)cc4)c3c2)cc1)N(C)C] |
| 197 | 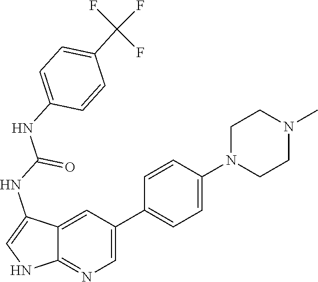 [SMILES: CN1CCN(c2ccc(-c3cnc4[nH]cc(NC(=O)Nc5ccc(C(F)(F)F)cc5)c4c3)cc2)CC1] |
| 198 | 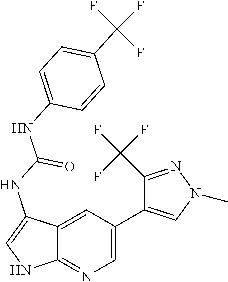 [SMILES: Cn1cc(-c2cnc3[nH]cc(NC(=O)Nc4ccc(C(F)(F)F)cc4)c3c2)c(C(F)(F)F)n1] |
| 199 | 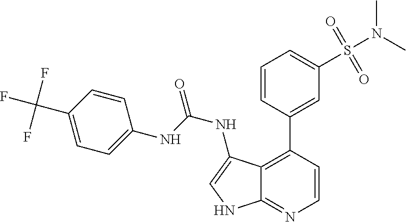 [SMILES: CN(C)S(=O)(=O)c1cccc(-c2ccnc3[nH]cc(NC(=O)Nc4ccc(C(F)(F)F)cc4)c23)c1] |
| 200 | 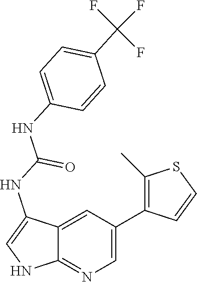 [SMILES: Cc1sccc1-c1cnc2[nH]cc(NC(=O)Nc3ccc(C(F)(F)F)cc3)c2c1] |
| 201 | 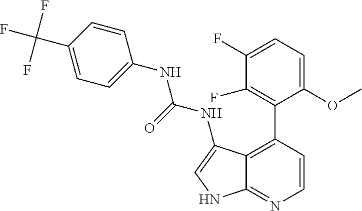 [SMILES: COc1ccc(F)c(F)c1-c1ccnc2[nH]cc(NC(=O)Nc3ccc(C(F)(F)F)cc3)c12] |
| 202 | 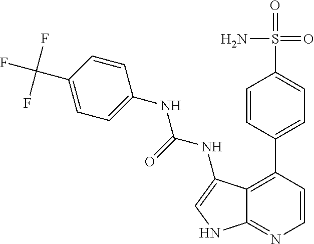 [SMILES: NS(=O)(=O)c1ccc(-c2ccnc3[nH]cc(NC(=O)Nc4ccc(C(F)(F)F)cc4)c23)cc1] |
| 203 | 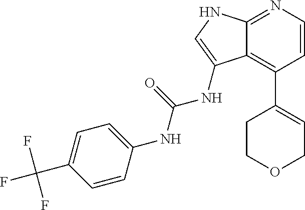 [SMILES: O=C(Nc1ccc(C(F)(F)F)cc1)Nc1c[nH]c2nccc(C3=CCOCC3)c12] |
| 204 | 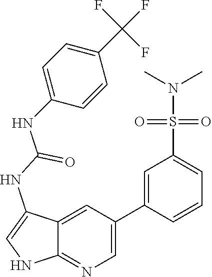 [SMILES: CN(C)S(=O)(=O)c1cccc(-c2cnc3[nH]cc(NC(=O)Nc4ccc(C(F)(F)F)cc4)c3c2)c1] |
| 205 | 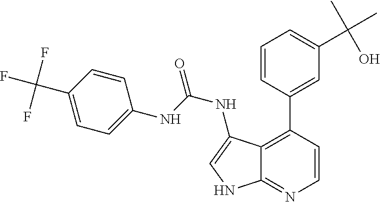 [SMILES: CC(C)(O)c1cccc(-c2ccnc3[nH]cc(NC(=O)Nc4ccc(C(F)(F)F)cc4)c23)c1] |
| 206 | 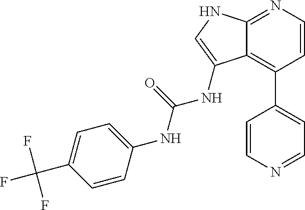 [SMILES: O=C(Nc1ccc(C(F)(F)F)cc1)Nc1c[nH]c2nccc(-c3ccncc3)c12] |
| 207 | 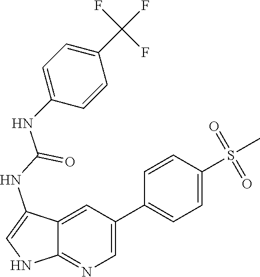 [SMILES: CS(=O)(=O)c1ccc(-c2cnc3[nH]cc(NC(=O)Nc4ccc(C(F)(F)F)cc4)c3c2)cc1] |
| 208 | 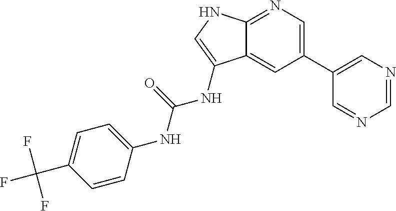 [SMILES: O=C(Nc1ccc(C(F)(F)F)cc1)Nc1c[nH]c2ncc(-c3cncnc3)cc12] |
| 209 | 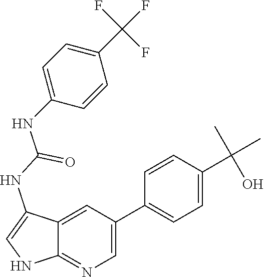 [SMILES: CC(C)(O)c1ccc(-c2cnc3[nH]cc(NC(=O)Nc4ccc(C(F)(F)F)cc4)c3c2)cc1] |
| 210 | 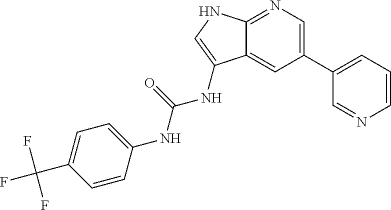 [SMILES: O=C(Nc1ccc(C(F)(F)F)cc1)Nc1c[nH]c2ncc(-c3cccnc3)cc12] |
| 211 | 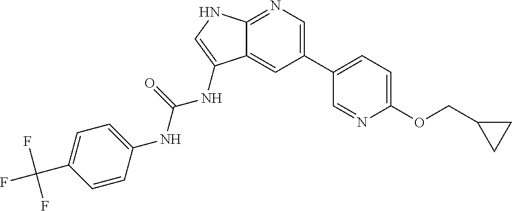 [SMILES: O=C(Nc1ccc(C(F)(F)F)cc1)Nc1c[nH]c2ncc(-c3ccc(OCC4CC4)nc3)cc12] |
| 212 | 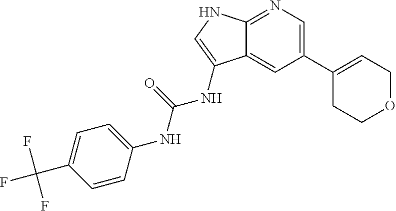 [SMILES: O=C(Nc1ccc(C(F)(F)F)cc1)Nc1c[nH]c2ncc(C3=CCOCC3)cc12] |
| 213 | 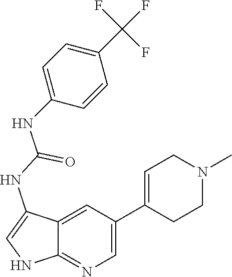 [SMILES: CN1CC=C(c2cnc3[nH]cc(NC(=O)Nc4ccc(C(F)(F)F)cc4)c3c2)CC1] |
| 214 | 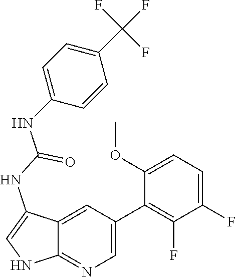 [SMILES: COc1ccc(F)c(F)c1-c1cnc2[nH]cc(NC(=O)Nc3ccc(C(F)(F)F)cc3)c2c1] |
| 215 | 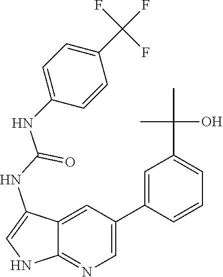 [SMILES: CC(C)(O)c1cccc(-c2cnc3[nH]cc(NC(=O)Nc4ccc(C(F)(F)F)cc4)c3c2)c1] |
| 216 | 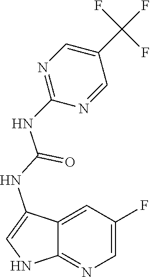 [SMILES: O=C(Nc1ncc(C(F)(F)F)cn1)Nc1c[nH]c2ncc(F)cc12] |
| 217 | 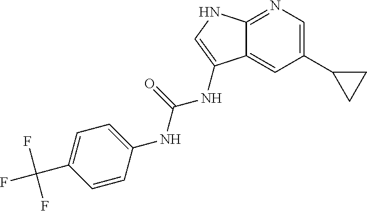 [SMILES: O=C(Nc1ccc(C(F)(F)F)cc1)Nc1c[nH]c2ncc(C3CC3)cc12] |
| 218 | 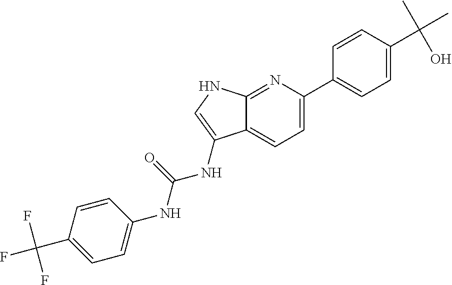 [SMILES: CC(C)(O)c1ccc(-c2ccc3c(NC(=O)Nc4ccc(C(F)(F)F)cc4)c[nH]c3n2)cc1] |
| 219 | 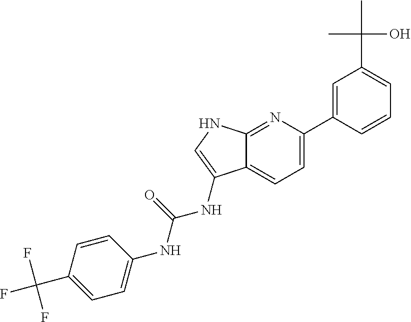 [SMILES: CC(C)(O)c1cccc(-c2ccc3c(NC(=O)Nc4ccc(C(F)(F)F)cc4)c[nH]c3n2)c1] |
| 220 | 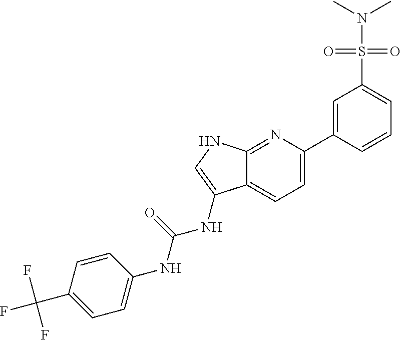 [SMILES: CN(C)S(=O)(=O)c1cccc(-c2ccc3c(NC(=O)Nc4ccc(C(F)(F)F)cc4)c[nH]c3n2)c1] |
| 221 | 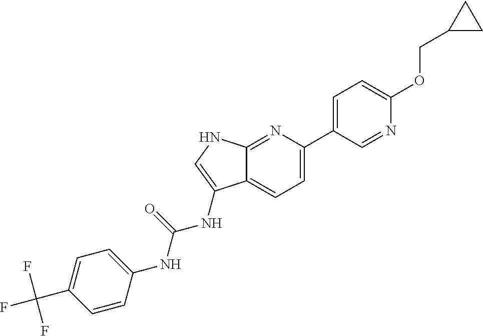 [SMILES: O=C(Nc1ccc(C(F)(F)F)cc1)Nc1c[nH]c2nc(-c3ccc(OCC4CC4)nc3)ccc12] |
| 222 | 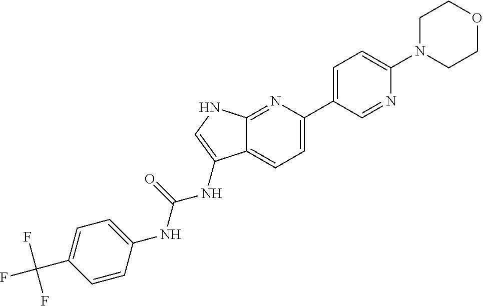 [SMILES: O=C(Nc1ccc(C(F)(F)F)cc1)Nc1c[nH]c2nc(-c3ccc(N4CCOCC4)nc3)ccc12] |
| 223 | 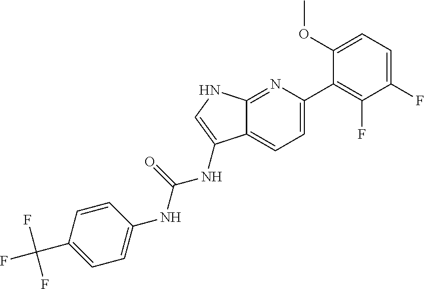 [SMILES: COc1ccc(F)c(F)c1-c1ccc2c(NC(=O)Nc3ccc(C(F)(F)F)cc3)c[nH]c2n1] |
| 224 | 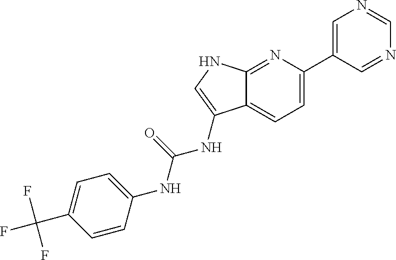 [SMILES: O=C(Nc1ccc(C(F)(F)F)cc1)Nc1c[nH]c2nc(-c3cncnc3)ccc12] |
| 225 | 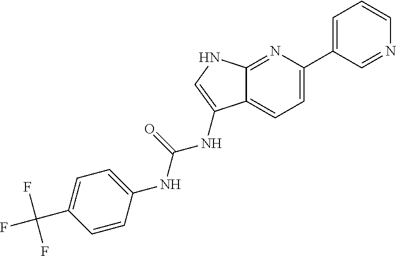 [SMILES: O=C(Nc1ccc(C(F)(F)F)cc1)Nc1c[nH]c2nc(-c3cccnc3)ccc12] |
| 226 | 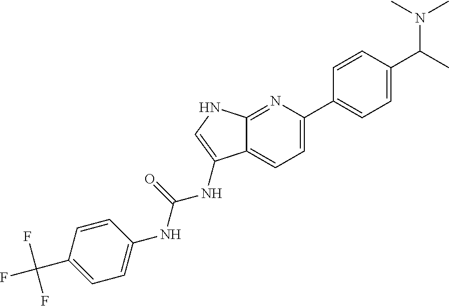 [SMILES: CC(c1ccc(-c2ccc3c(NC(=O)Nc4ccc(C(F)(F)F)cc4)c[nH]c3n2)cc1)N(C)C] |
| 227 | 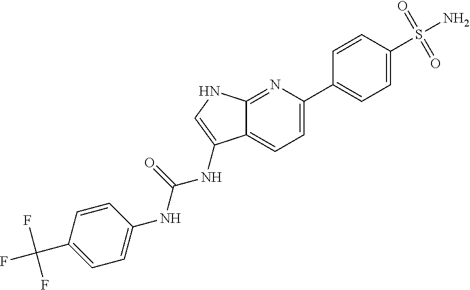 [SMILES: NS(=O)(=O)c1ccc(-c2ccc3c(NC(=O)Nc4ccc(C(F)(F)F)cc4)c[nH]c3n2)cc1] |
| 228 | 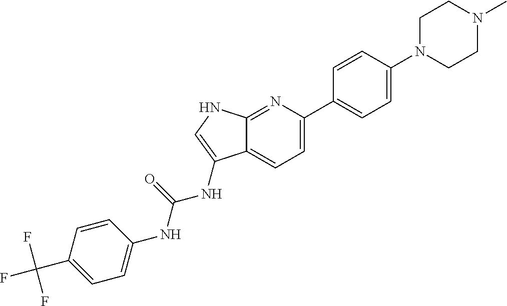 [SMILES: CN1CCN(c2ccc(-c3ccc4c(NC(=O)Nc5ccc(C(F)(F)F)cc5)c[nH]c4n3)cc2)CC1] |
| 229 | 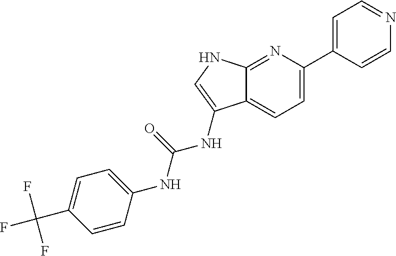 [SMILES: O=C(Nc1ccc(C(F)(F)F)cc1)Nc1c[nH]c2nc(-c3ccncc3)ccc12] |
| 230 | 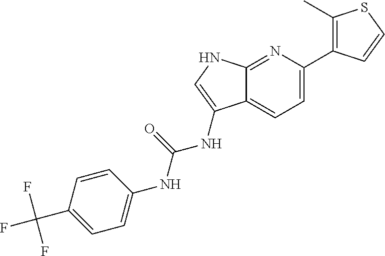 [SMILES: Cc1sccc1-c1ccc2c(NC(=O)Nc3ccc(C(F)(F)F)cc3)c[nH]c2n1] |
| 231 | 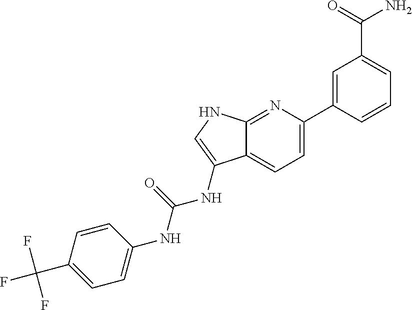 [SMILES: NC(=O)c1cccc(-c2ccc3c(NC(=O)Nc4ccc(C(F)(F)F)cc4)c[nH]c3n2)c1] |
| 232 | 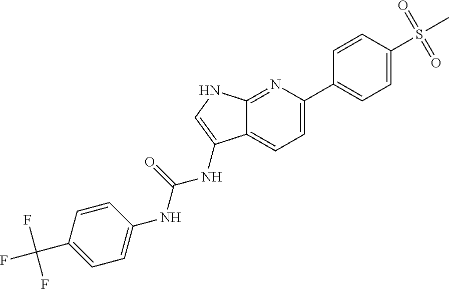 [SMILES: CS(=O)(=O)c1ccc(-c2ccc3c(NC(=O)Nc4ccc(C(F)(F)F)cc4)c[nH]c3n2)cc1] |
| 233 | 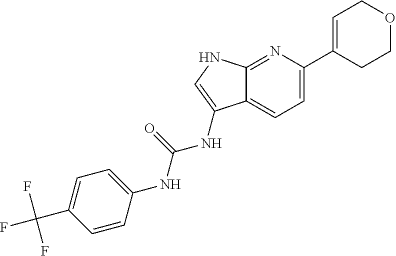 [SMILES: O=C(Nc1ccc(C(F)(F)F)cc1)Nc1c[nH]c2nc(C3=CCOCC3)ccc12] |
| 234 | 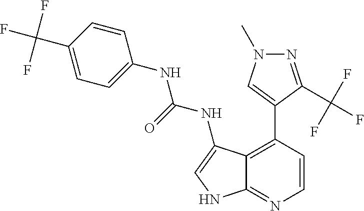 [SMILES: Cn1cc(-c2ccnc3[nH]cc(NC(=O)Nc4ccc(C(F)(F)F)cc4)c23)c(C(F)(F)F)n1] |
| 235 | 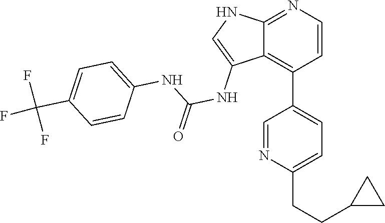 [SMILES: O=C(Nc1ccc(C(F)(F)F)cc1)Nc1c[nH]c2nccc(-c3ccc(CCC4CC4)nc3)c12] |
| 236 | 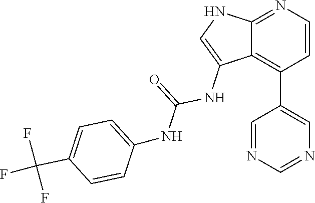 [SMILES: O=C(Nc1ccc(C(F)(F)F)cc1)Nc1c[nH]c2nccc(-c3cncnc3)c12] |
| 237 | 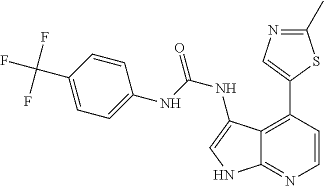 [SMILES: Cc1ncc(-c2ccnc3[nH]cc(NC(=O)Nc4ccc(C(F)(F)F)cc4)c23)s1] |
| 238 | 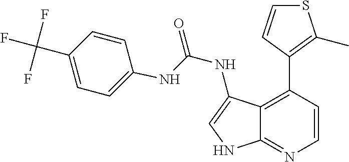 [SMILES: Cc1sccc1-c1ccnc2[nH]cc(NC(=O)Nc3ccc(C(F)(F)F)cc3)c12] |
| 239 | 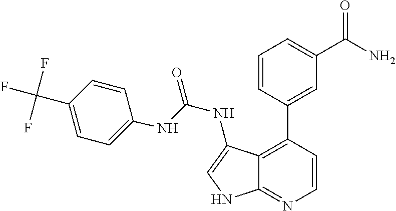 [SMILES: NC(=O)c1cccc(-c2ccnc3[nH]cc(NC(=O)Nc4ccc(C(F)(F)F)cc4)c23)c1] |
| 240 | 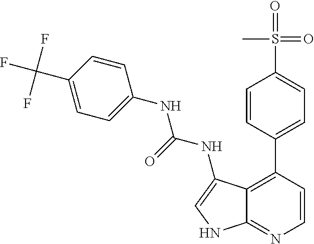 [SMILES: CS(=O)(=O)c1ccc(-c2ccnc3[nH]cc(NC(=O)Nc4ccc(C(F)(F)F)cc4)c23)cc1] |
| 241 | 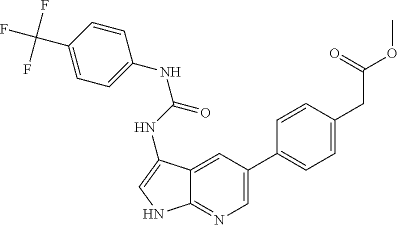 [SMILES: COC(=O)Cc1ccc(-c2cnc3[nH]cc(NC(=O)Nc4ccc(C(F)(F)F)cc4)c3c2)cc1] |
| 242 | 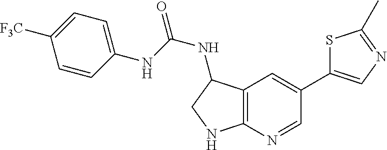 [SMILES: Cc1ncc(-c2cnc3c(c2)C(NC(=O)Nc2ccc(C(F)(F)F)cc2)CN3)s1] |
| 243 | 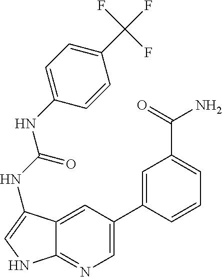 [SMILES: NC(=O)c1cccc(-c2cnc3[nH]cc(NC(=O)Nc4ccc(C(F)(F)F)cc4)c3c2)c1] |
| 244 | 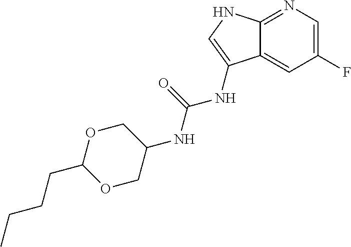 [SMILES: CCCCC1OCC(NC(=O)Nc2c[nH]c3ncc(F)cc23)CO1] |
| 245 | 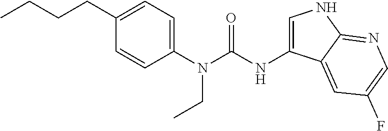 [SMILES: CCCCc1ccc(N(CC)C(=O)Nc2c[nH]c3ncc(F)cc23)cc1] |
| 246 | 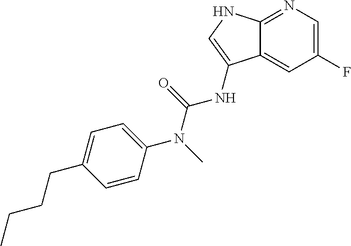 [SMILES: CCCCc1ccc(N(C)C(=O)Nc2c[nH]c3ncc(F)cc23)cc1] |
| 247 | 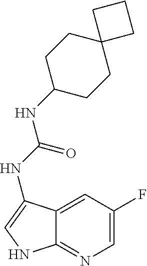 [SMILES: O=C(Nc1c[nH]c2ncc(F)cc12)NC1CCC2(CCC2)CC1] |
| 248 | 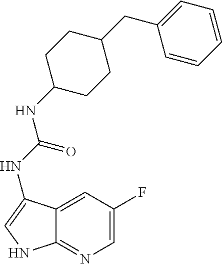 [SMILES: O=C(Nc1c[nH]c2ncc(F)cc12)NC1CCC(Cc2ccccc2)CC1] |
| 249 | 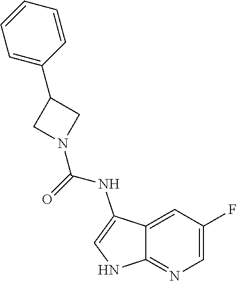 [SMILES: O=C(Nc1c[nH]c2ncc(F)cc12)N1CC(c2ccccc2)C1] |
| 250 | 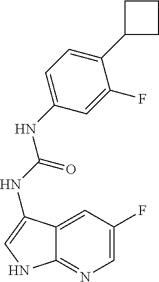 [SMILES: O=C(Nc1ccc(C2CCC2)c(F)c1)Nc1c[nH]c2ncc(F)cc12] |
| 251 | 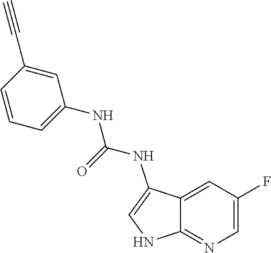 [SMILES: C#Cc1cccc(NC(=O)Nc2c[nH]c3ncc(F)cc23)c1] |
| 252 | 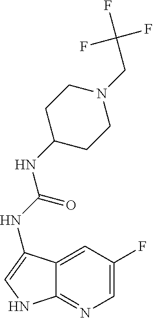 [SMILES: O=C(Nc1c[nH]c2ncc(F)cc12)NC1CCN(CC(F)(F)F)CC1] |
| 253 | 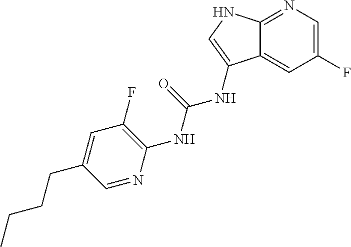 [SMILES: CCCCc1cnc(NC(=O)Nc2c[nH]c3ncc(F)cc23)c(F)c1] |
| 254 | 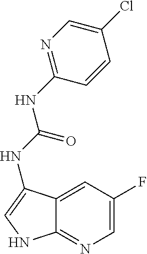 [SMILES: O=C(Nc1ccc(Cl)cn1)Nc1c[nH]c2ncc(F)cc12] |
| 255 | 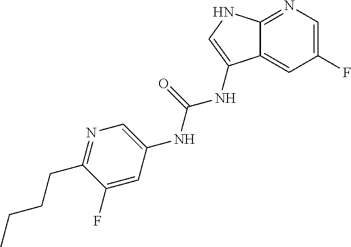 [SMILES: CCCCc1ncc(NC(=O)Nc2c[nH]c3ncc(F)cc23)cc1F] |
| 256 | 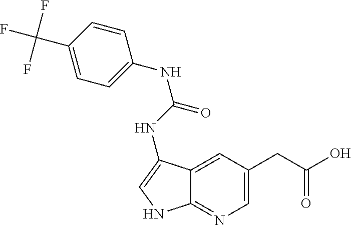 [SMILES: O=C(O)Cc1cnc2[nH]cc(NC(=O)Nc3ccc(C(F)(F)F)cc3)c2c1] |
| 257 | 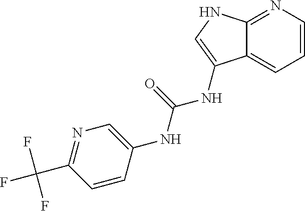 [SMILES: O=C(Nc1ccc(C(F)(F)F)nc1)Nc1c[nH]c2ncccc12] |
| 258 | 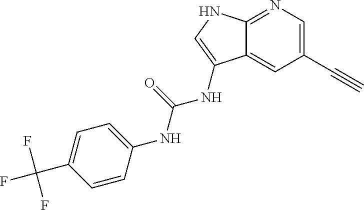 [SMILES: C#Cc1cnc2[nH]cc(NC(=O)Nc3ccc(C(F)(F)F)cc3)c2c1] |
| 259 | 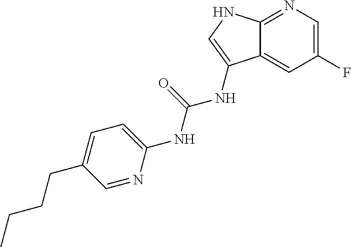 [SMILES: CCCCc1ccc(NC(=O)Nc2c[nH]c3ncc(F)cc23)nc1] |
| 260 | 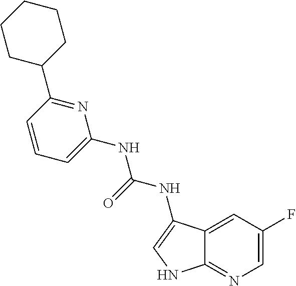 [SMILES: O=C(Nc1cccc(C2CCCCC2)n1)Nc1c[nH]c2ncc(F)cc12] |
| 261 | 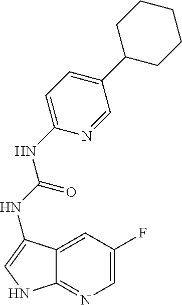 [SMILES: O=C(Nc1ccc(C2CCCCC2)cn1)Nc1c[nH]c2ncc(F)cc12] |
| 262 | 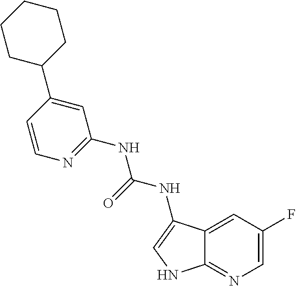 [SMILES: O=C(Nc1cc(C2CCCCC2)ccn1)Nc1c[nH]c2ncc(F)cc12] |
| 263 | 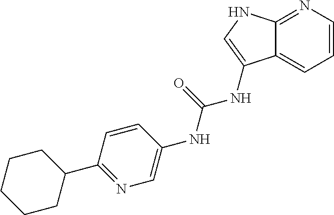 [SMILES: O=C(Nc1ccc(C2CCCCC2)nc1)Nc1c[nH]c2ncccc12] |
| 264 | 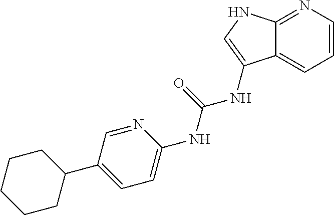 [SMILES: O=C(Nc1ccc(C2CCCCC2)cn1)Nc1c[nH]c2ncccc12] |
| 265 | 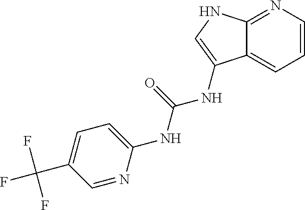 [SMILES: O=C(Nc1ccc(C(F)(F)F)cn1)Nc1c[nH]c2ncccc12] |
| 266 | 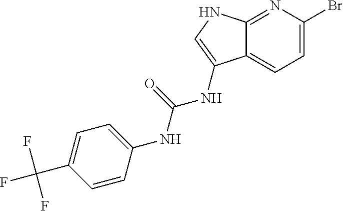 [SMILES: O=C(Nc1ccc(C(F)(F)F)cc1)Nc1c[nH]c2nc(Br)ccc12] |
| 267 | 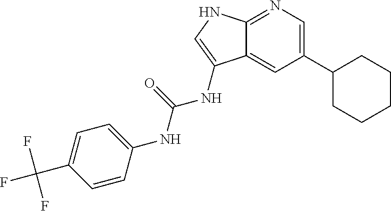 [SMILES: O=C(Nc1ccc(C(F)(F)F)cc1)Nc1c[nH]c2ncc(C3CCCCC3)cc12] |
| 268 | 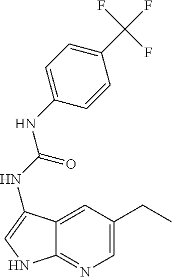 [SMILES: CCc1cnc2[nH]cc(NC(=O)Nc3ccc(C(F)(F)F)cc3)c2c1] |
| 269 | 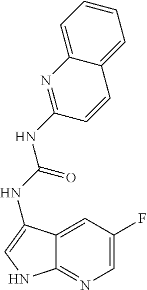 [SMILES: O=C(Nc1ccc2ccccc2n1)Nc1c[nH]c2ncc(F)cc12] |
| 270 | 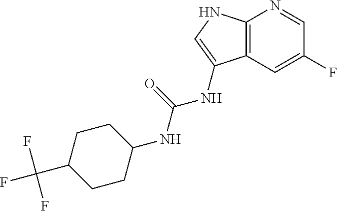 [SMILES: O=C(Nc1c[nH]c2ncc(F)cc12)NC1CCC(C(F)(F)F)CC1] |
| 271 | 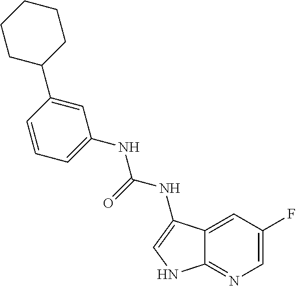 [SMILES: O=C(Nc1cccc(C2CCCCC2)c1)Nc1c[nH]c2ncc(F)cc12] |
| 272 | 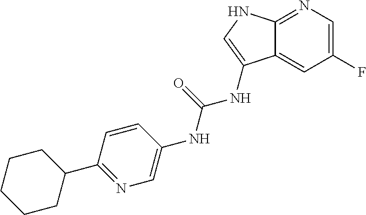 [SMILES: O=C(Nc1ccc(C2CCCCC2)nc1)Nc1c[nH]c2ncc(F)cc12] |
| 273 | 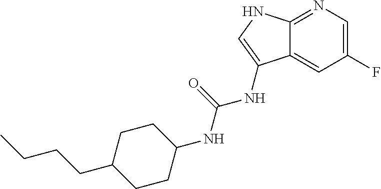 [SMILES: CCCCC1CCC(NC(=O)Nc2c[nH]c3ncc(F)cc23)CC1] |
| 274 | 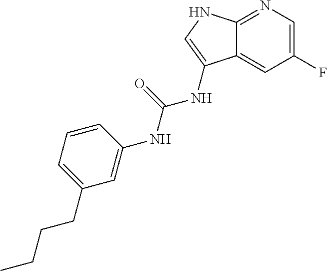 [SMILES: CCCCc1cccc(NC(=O)Nc2c[nH]c3ncc(F)cc23)c1] |
| 275 | 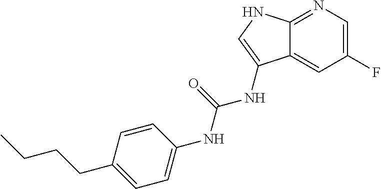 [SMILES: CCCCc1ccc(NC(=O)Nc2c[nH]c3ncc(F)cc23)cc1] |
| 276 | 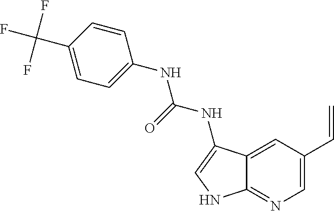 [SMILES: C=Cc1cnc2[nH]cc(NC(=O)Nc3ccc(C(F)(F)F)cc3)c2c1] |
| 277 | 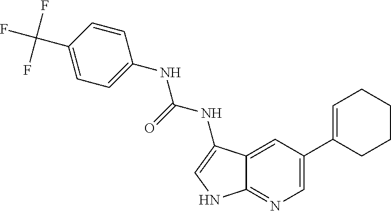 [SMILES: O=C(Nc1ccc(C(F)(F)F)cc1)Nc1c[nH]c2ncc(C3=CCCCC3)cc12] |
| 278 | 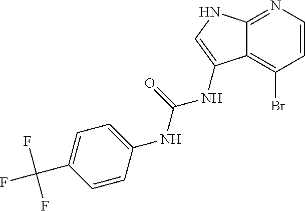 [SMILES: O=C(Nc1ccc(C(F)(F)F)cc1)Nc1c[nH]c2nccc(Br)c12] |
| 279 | 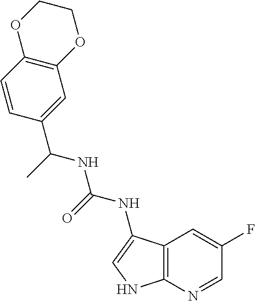 [SMILES: CC(NC(=O)Nc1c[nH]c2ncc(F)cc12)c1ccc2c(c1)OCCO2] |
| 280 | 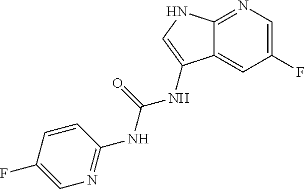 [SMILES: O=C(Nc1ccc(F)cn1)Nc1c[nH]c2ncc(F)cc12] |
| 281 | 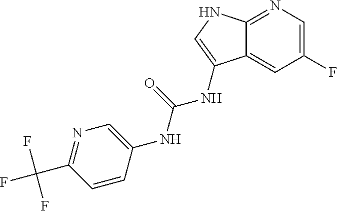 [SMILES: O=C(Nc1ccc(C(F)(F)F)nc1)Nc1c[nH]c2ncc(F)cc12] |
| 282 | 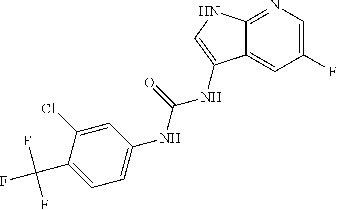 [SMILES: O=C(Nc1ccc(C(F)(F)F)c(Cl)c1)Nc1c[nH]c2ncc(F)cc12] |
| 283 | 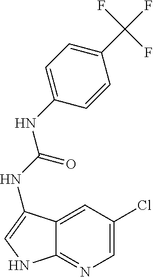 [SMILES: O=C(Nc1ccc(C(F)(F)F)cc1)Nc1c[nH]c2ncc(Cl)cc12] |
| 284 | 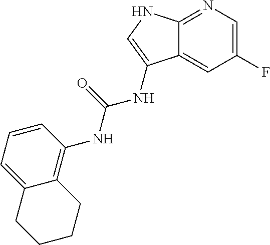 [SMILES: O=C(Nc1cccc2c1CCCC2)Nc1c[nH]c2ncc(F)cc12] |
| 285 | 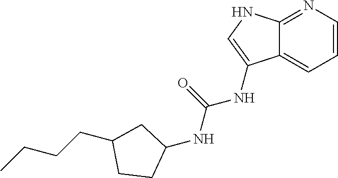 [SMILES: CCCCC1CCC(NC(=O)Nc2c[nH]c3ncccc23)C1] |
| 286 | 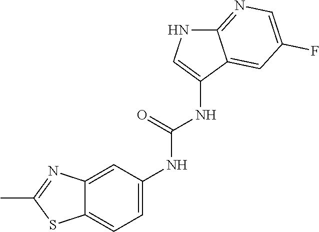 [SMILES: Cc1nc2cc(NC(=O)Nc3c[nH]c4ncc(F)cc34)ccc2s1] |
| 287 | 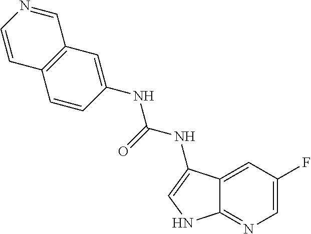 [SMILES: O=C(Nc1ccc2ccncc2c1)Nc1c[nH]c2ncc(F)cc12] |
| 288 | 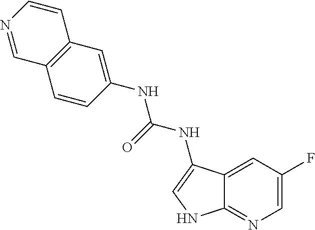 [SMILES: O=C(Nc1ccc2cnccc2c1)Nc1c[nH]c2ncc(F)cc12] |
| 289 | 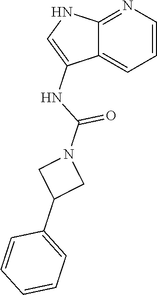 [SMILES: O=C(Nc1c[nH]c2ncccc12)N1CC(c2ccccc2)C1] |
| 290 | 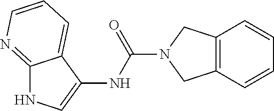 [SMILES: O=C(Nc1c[nH]c2ncccc12)N1Cc2ccccc2C1] |
| 291 | 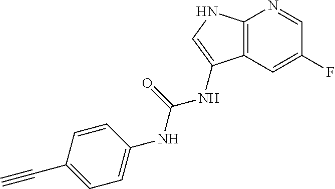 [SMILES: C#Cc1ccc(NC(=O)Nc2c[nH]c3ncc(F)cc23)cc1] |
| 292 | 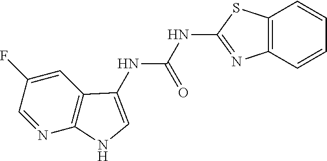 [SMILES: O=C(Nc1nc2ccccc2s1)Nc1c[nH]c2ncc(F)cc12] |
| 293 | 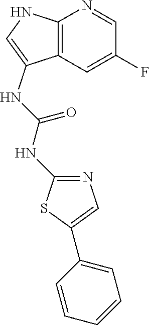 [SMILES: O=C(Nc1ncc(-c2ccccc2)s1)Nc1c[nH]c2ncc(F)cc12] |
| 294 | 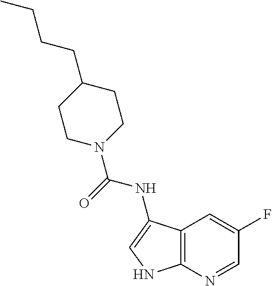 [SMILES: CCCCC1CCN(C(=O)Nc2c[nH]c3ncc(F)cc23)CC1] |
| 295 | 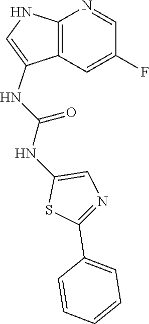 [SMILES: O=C(Nc1cnc(-c2ccccc2)s1)Nc1c[nH]c2ncc(F)cc12] |
| 296 | 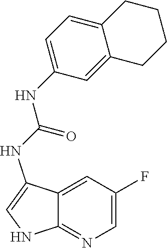 [SMILES: O=C(Nc1ccc2c(c1)CCCC2)Nc1c[nH]c2ncc(F)cc12] |
| 297 | 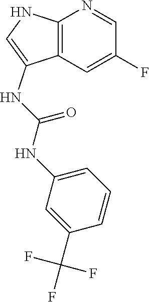 [SMILES: O=C(Nc1cccc(C(F)(F)F)c1)Nc1c[nH]c2ncc(F)cc12] |
| 298 | 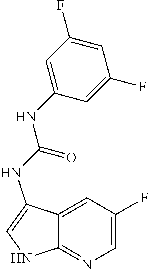 [SMILES: O=C(Nc1cc(F)cc(F)c1)Nc1c[nH]c2ncc(F)cc12] |
| 299 | 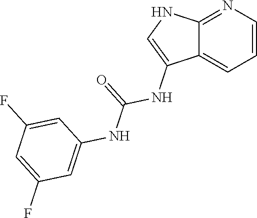 [SMILES: O=C(Nc1cc(F)cc(F)c1)Nc1c[nH]c2ncccc12] |
| 300 | 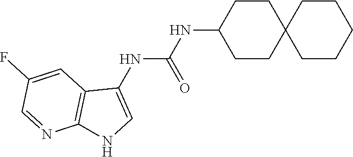 [SMILES: O=C(Nc1c[nH]c2ncc(F)cc12)NC1CCC2(CCCCC2)CC1] |
| 301 | 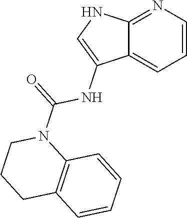 [SMILES: O=C(Nc1c[nH]c2ncccc12)N1CCCc2ccccc21] |
| 302 | 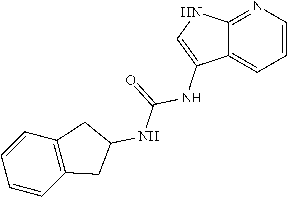 [SMILES: O=C(Nc1c[nH]c2ncccc12)NC1Cc2ccccc2C1] |
| 303 | 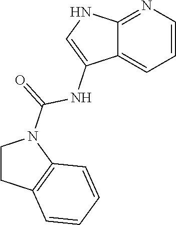 [SMILES: O=C(Nc1c[nH]c2ncccc12)N1CCc2ccccc21] |
| 304 | 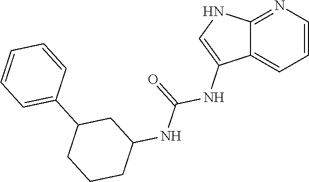 [SMILES: O=C(Nc1c[nH]c2ncccc12)NC1CCCC(c2ccccc2)C1] |
| 305 | 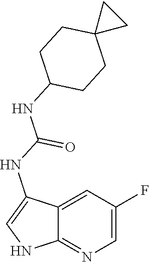 [SMILES: O=C(Nc1c[nH]c2ncc(F)cc12)NC1CCC2(CC1)CC2] |
| 306 | 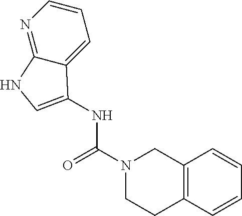 [SMILES: O=C(Nc1c[nH]c2ncccc12)N1CCc2ccccc2C1] |
| 307 | 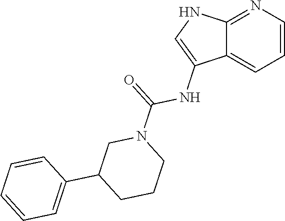 [SMILES: O=C(Nc1c[nH]c2ncccc12)N1CCCC(c2ccccc2)C1] |
| 308 | 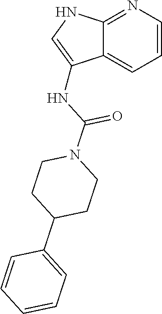 [SMILES: O=C(Nc1c[nH]c2ncccc12)N1CCC(c2ccccc2)CC1] |
| 309 | 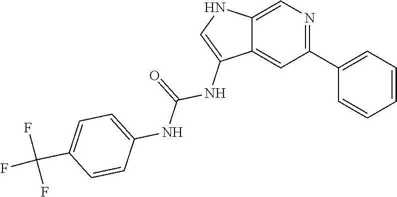 [SMILES: O=C(Nc1ccc(C(F)(F)F)cc1)Nc1c[nH]c2cnc(-c3ccccc3)cc12] |
| 310 | 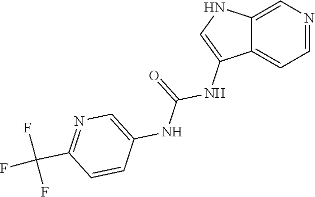 [SMILES: O=C(Nc1ccc(C(F)(F)F)nc1)Nc1c[nH]c2cnccc12] |
| 311 | 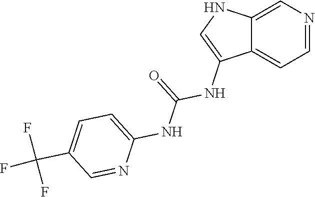 [SMILES: O=C(Nc1ccc(C(F)(F)F)cn1)Nc1c[nH]c2cnccc12] |
| 312 | 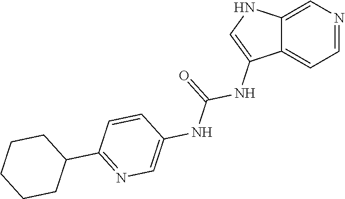 [SMILES: O=C(Nc1ccc(C2CCCCC2)nc1)Nc1c[nH]c2cnccc12] |
| 313 | 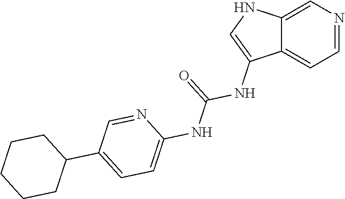 [SMILES: O=C(Nc1ccc(C2CCCCC2)cn1)Nc1c[nH]c2cnccc12] |
| 314 | 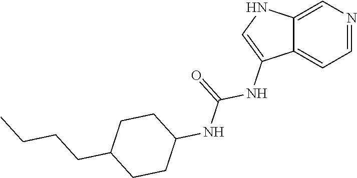 [SMILES: CCCCC1CCC(NC(=O)Nc2c[nH]c3cnccc23)CC1] |
| 315 | 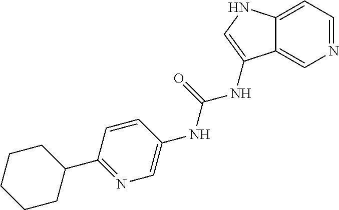 [SMILES: O=C(Nc1ccc(C2CCCCC2)nc1)Nc1c[nH]c2ccncc12] |
| 316 | 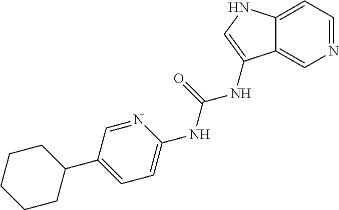 [SMILES: O=C(Nc1ccc(C2CCCCC2)cn1)Nc1c[nH]c2ccncc12] |
| 317 | 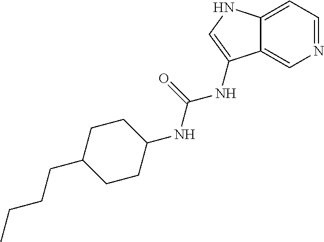 [SMILES: CCCCC1CCC(NC(=O)Nc2c[nH]c3ccncc23)CC1] |
| 318 | 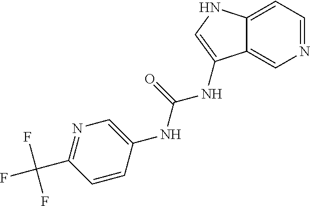 [SMILES: O=C(Nc1ccc(C(F)(F)F)nc1)Nc1c[nH]c2ccncc12] |
| 319 | 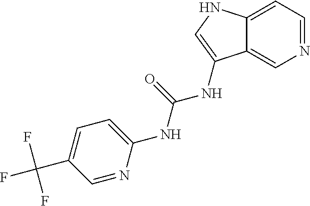 [SMILES: O=C(Nc1ccc(C(F)(F)F)cn1)Nc1c[nH]c2ccncc12] |
| 320 | 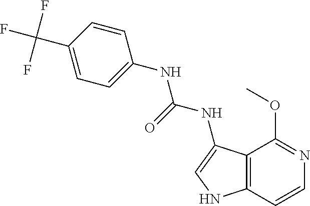 [SMILES: COc1nccc2[nH]cc(NC(=O)Nc3ccc(C(F)(F)F)cc3)c12] |
| 321 | 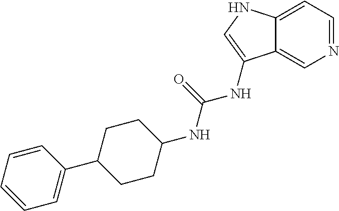 [SMILES: O=C(Nc1c[nH]c2ccncc12)NC1CCC(c2ccccc2)CC1] |
| 322 | 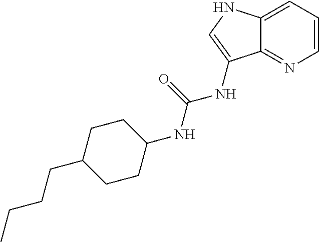 [SMILES: CCCCC1CCC(NC(=O)Nc2c[nH]c3cccnc23)CC1] |
| 323 | 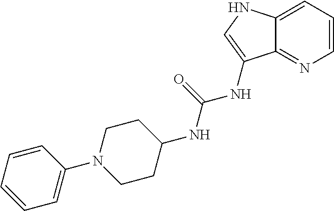 [SMILES: O=C(Nc1c[nH]c2cccnc12)NC1CCN(c2ccccc2)CC1] |
| 324 | 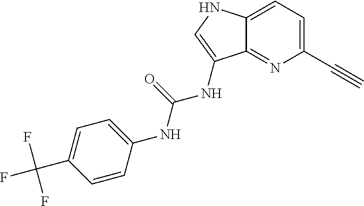 [SMILES: C#Cc1ccc2[nH]cc(NC(=O)Nc3ccc(C(F)(F)F)cc3)c2n1] |
| 325 | 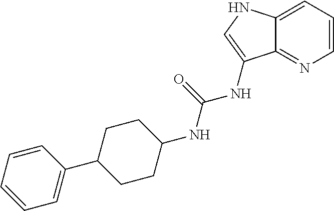 [SMILES: O=C(Nc1c[nH]c2cccnc12)NC1CCC(c2ccccc2)CC1] |
| 326 | 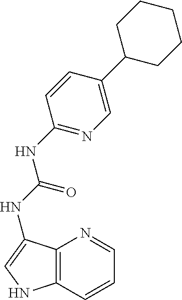 [SMILES: O=C(Nc1ccc(C2CCCCC2)cn1)Nc1c[nH]c2cccnc12] |
| 327 | 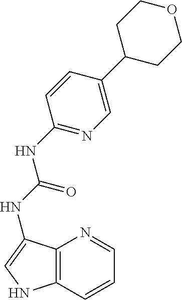 [SMILES: O=C(Nc1ccc(C2CCOCC2)cn1)Nc1c[nH]c2cccnc12] |
| 328 | 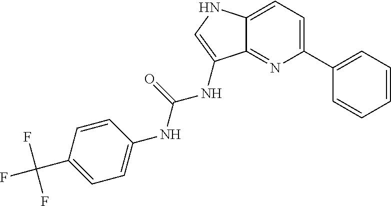 [SMILES: O=C(Nc1ccc(C(F)(F)F)cc1)Nc1c[nH]c2ccc(-c3ccccc3)nc12] |
| 329 | 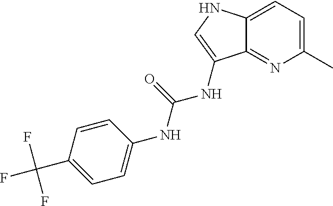 [SMILES: Cc1ccc2[nH]cc(NC(=O)Nc3ccc(C(F)(F)F)cc3)c2n1] |
| 330 | 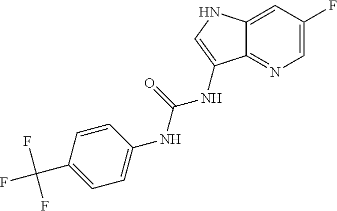 [SMILES: O=C(Nc1ccc(C(F)(F)F)cc1)Nc1c[nH]c2cc(F)cnc12] |
| 331 | 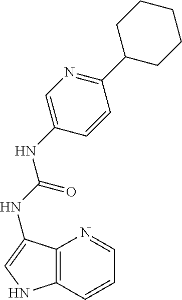 [SMILES: O=C(Nc1ccc(C2CCCCC2)nc1)Nc1c[nH]c2cccnc12] |
| 332 | 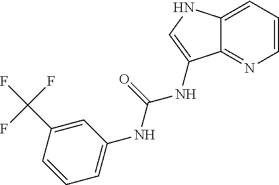 [SMILES: O=C(Nc1cccc(C(F)(F)F)c1)Nc1c[nH]c2cccnc12] |
| 333 | 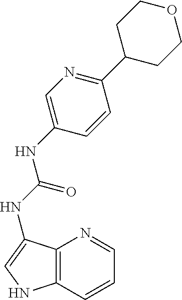 [SMILES: O=C(Nc1ccc(C2CCOCC2)nc1)Nc1c[nH]c2cccnc12] |
| 334 | 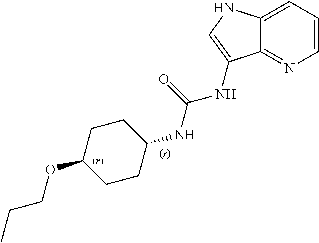 [SMILES: CCCO[C@H]1CC[C@H](NC(=O)Nc2c[nH]c3cccnc23)CC1] |
| 335 | 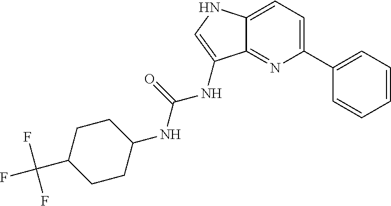 [SMILES: O=C(Nc1c[nH]c2ccc(-c3ccccc3)nc12)NC1CCC(C(F)(F)F)CC1] |
| 336 | 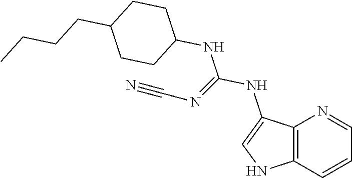 [SMILES: CCCCC1CCC(N/C(=N\C#N)Nc2c[nH]c3cccnc23)CC1] |
| 337 | 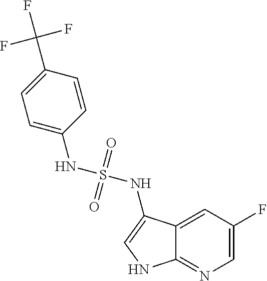 [SMILES: O=S(=O)(Nc1ccc(C(F)(F)F)cc1)Nc1c[nH]c2ncc(F)cc12] |
| 338 | 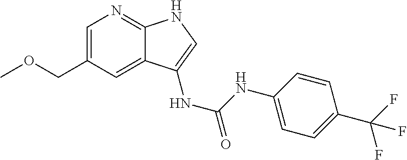 [SMILES: COCc1cnc2[nH]cc(NC(=O)Nc3ccc(C(F)(F)F)cc3)c2c1] |
| 339 | 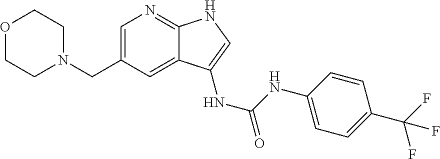 [SMILES: O=C(Nc1ccc(C(F)(F)F)cc1)Nc1c[nH]c2ncc(CN3CCOCC3)cc12] |
| 340 | 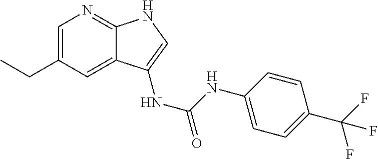 [SMILES: CCc1cnc2[nH]cc(NC(=O)Nc3ccc(C(F)(F)F)cc3)c2c1] |
| 341 | 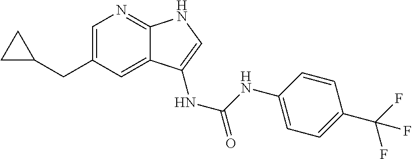 [SMILES: O=C(Nc1ccc(C(F)(F)F)cc1)Nc1c[nH]c2ncc(CC3CC3)cc12] |
| 342 | 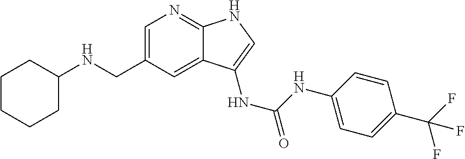 [SMILES: O=C(Nc1ccc(C(F)(F)F)cc1)Nc1c[nH]c2ncc(CNC3CCCCC3)cc12] |
| 343 | 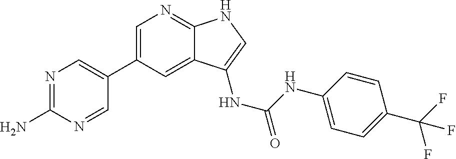 [SMILES: Nc1ncc(-c2cnc3[nH]cc(NC(=O)Nc4ccc(C(F)(F)F)cc4)c3c2)cn1] |
| 344 | 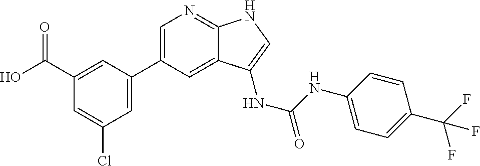 [SMILES: O=C(Nc1ccc(C(F)(F)F)cc1)Nc1c[nH]c2ncc(-c3cc(Cl)cc(C(=O)O)c3)cc12] |
| 345 | 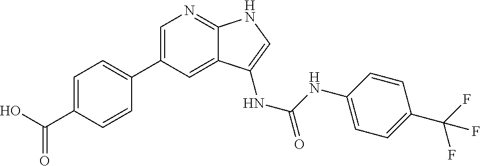 [SMILES: O=C(Nc1ccc(C(F)(F)F)cc1)Nc1c[nH]c2ncc(-c3ccc(C(=O)O)cc3)cc12] |
| 346 | 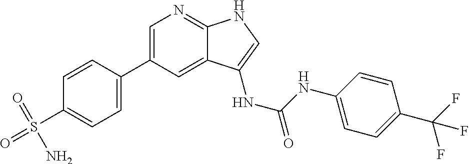 [SMILES: NS(=O)(=O)c1ccc(-c2cnc3[nH]cc(NC(=O)Nc4ccc(C(F)(F)F)cc4)c3c2)cc1] |
| 347 | 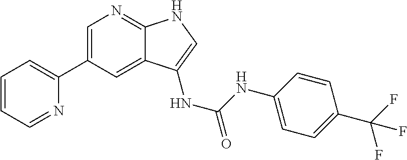 [SMILES: O=C(Nc1ccc(C(F)(F)F)cc1)Nc1c[nH]c2ncc(-c3ccccn3)cc12] |
| 348 | 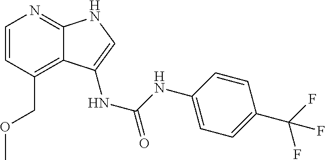 [SMILES: COCc1ccnc2[nH]cc(NC(=O)Nc3ccc(C(F)(F)F)cc3)c12] |
| 349 | 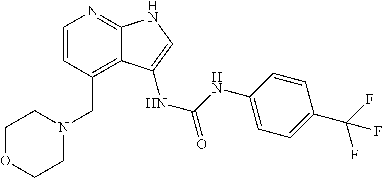 [SMILES: O=C(Nc1ccc(C(F)(F)F)cc1)Nc1c[nH]c2nccc(CN3CCOCC3)c12] |
| 350 | 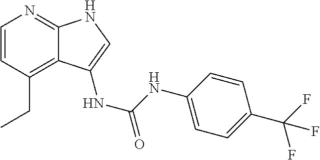 [SMILES: CCc1ccnc2[nH]cc(NC(=O)Nc3ccc(C(F)(F)F)cc3)c12] |
| 351 | 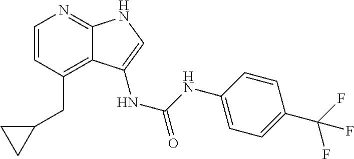 [SMILES: O=C(Nc1ccc(C(F)(F)F)cc1)Nc1c[nH]c2nccc(CC3CC3)c12] |
| 352 | 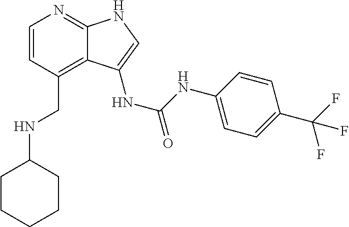 [SMILES: O=C(Nc1ccc(C(F)(F)F)cc1)Nc1c[nH]c2nccc(CNC3CCCCC3)c12] |
| 353 | 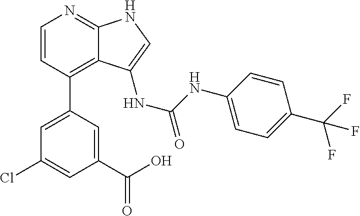 [SMILES: O=C(Nc1ccc(C(F)(F)F)cc1)Nc1c[nH]c2nccc(-c3cc(Cl)cc(C(=O)O)c3)c12] |
| 354 | 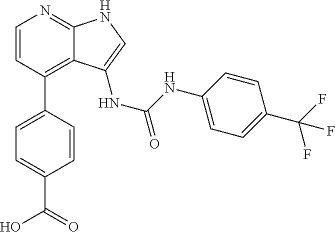 [SMILES: O=C(Nc1ccc(C(F)(F)F)cc1)Nc1c[nH]c2nccc(-c3ccc(C(=O)O)cc3)c12] |
| 355 | 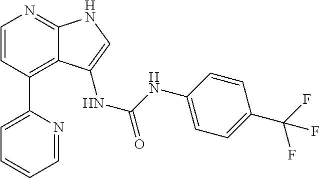 [SMILES: O=C(Nc1ccc(C(F)(F)F)cc1)Nc1c[nH]c2nccc(-c3ccccn3)c12] |
| 356 | 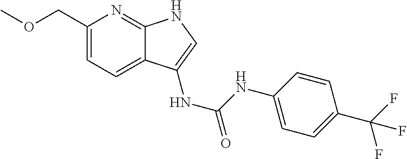 [SMILES: COCc1ccc2c(NC(=O)Nc3ccc(C(F)(F)F)cc3)c[nH]c2n1] |
| 357 | 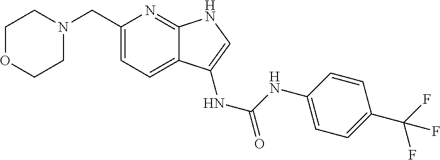 [SMILES: O=C(Nc1ccc(C(F)(F)F)cc1)Nc1c[nH]c2nc(CN3CCOCC3)ccc12] |
| 358 | 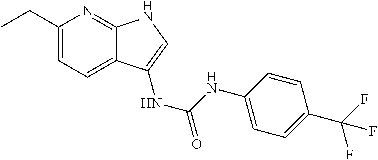 [SMILES: CCc1ccc2c(NC(=O)Nc3ccc(C(F)(F)F)cc3)c[nH]c2n1] |
| 359 | 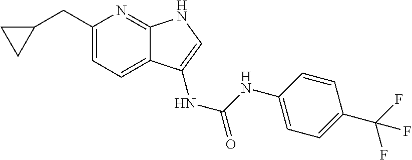 [SMILES: O=C(Nc1ccc(C(F)(F)F)cc1)Nc1c[nH]c2nc(CC3CC3)ccc12] |
| 360 | 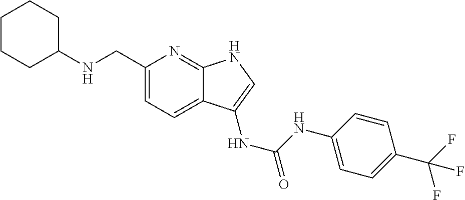 [SMILES: O=C(Nc1ccc(C(F)(F)F)cc1)Nc1c[nH]c2nc(CNC3CCCCC3)ccc12] |
| 361 | 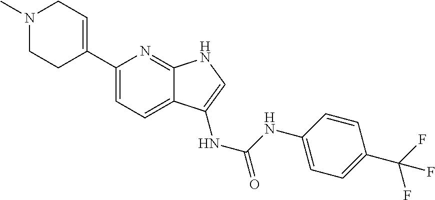 [SMILES: CN1CC=C(c2ccc3c(NC(=O)Nc4ccc(C(F)(F)F)cc4)c[nH]c3n2)CC1] |
| 362 | 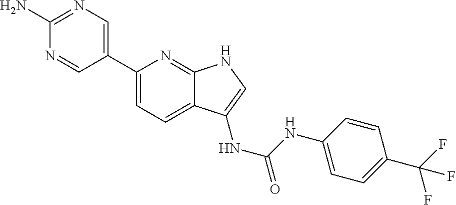 [SMILES: Nc1ncc(-c2ccc3c(NC(=O)Nc4ccc(C(F)(F)F)cc4)c[nH]c3n2)cn1] |
| 363 | 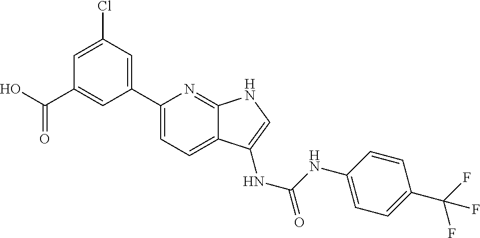 [SMILES: O=C(Nc1ccc(C(F)(F)F)cc1)Nc1c[nH]c2nc(-c3cc(Cl)cc(C(=O)O)c3)ccc12] |
| 364 | 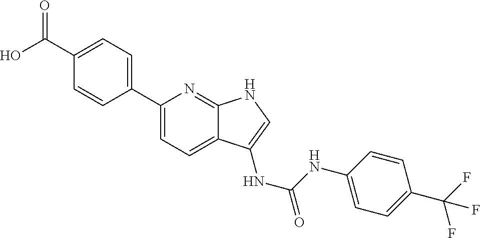 [SMILES: O=C(Nc1ccc(C(F)(F)F)cc1)Nc1c[nH]c2nc(-c3ccc(C(=O)O)cc3)ccc12] |
| 365 | 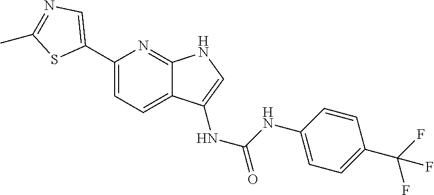 [SMILES: Cc1ncc(-c2ccc3c(NC(=O)Nc4ccc(C(F)(F)F)cc4)c[nH]c3n2)s1] |
| 366 | 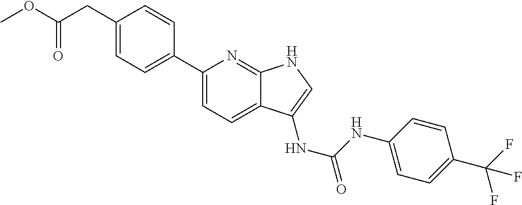 [SMILES: COC(=O)Cc1ccc(-c2ccc3c(NC(=O)Nc4ccc(C(F)(F)F)cc4)c[nH]c3n2)cc1] |
| 367 | 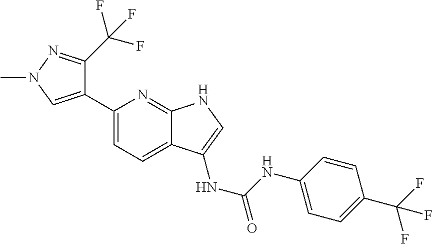 [SMILES: Cn1cc(-c2ccc3c(NC(=O)Nc4ccc(C(F)(F)F)cc4)c[nH]c3n2)c(C(F)(F)F)n1] |
| 368 | 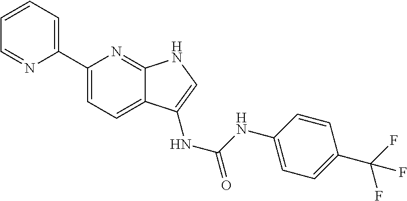 [SMILES: O=C(Nc1ccc(C(F)(F)F)cc1)Nc1c[nH]c2nc(-c3ccccn3)ccc12] |
| 369 | 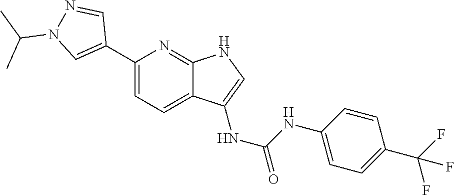 [SMILES: CC(C)n1cc(-c2ccc3c(NC(=O)Nc4ccc(C(F)(F)F)cc4)c[nH]c3n2)cn1] |
| 370 | 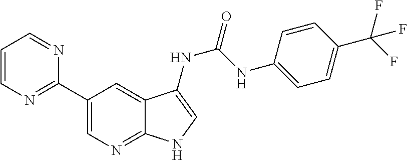 [SMILES: O=C(Nc1ccc(C(F)(F)F)cc1)Nc1c[nH]c2ncc(-c3ncccn3)cc12] |
| 371 | 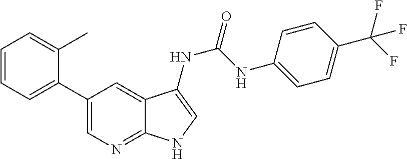 [SMILES: Cc1ccccc1-c1cnc2[nH]cc(NC(=O)Nc3ccc(C(F)(F)F)cc3)c2c1] |
| 372 | 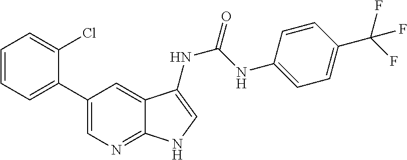 [SMILES: O=C(Nc1ccc(C(F)(F)F)cc1)Nc1c[nH]c2ncc(-c3ccccc3Cl)cc12] |
| 373 | 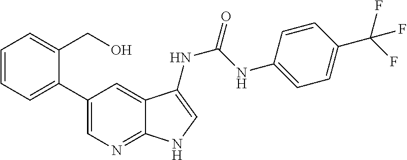 [SMILES: O=C(Nc1ccc(C(F)(F)F)cc1)Nc1c[nH]c2ncc(-c3ccccc3CO)cc12] |
| 374 | 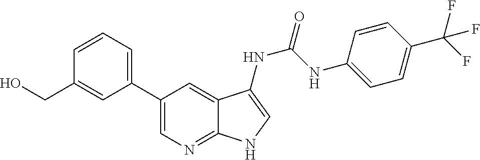 [SMILES: O=C(Nc1ccc(C(F)(F)F)cc1)Nc1c[nH]c2ncc(-c3cccc(CO)c3)cc12] |
| 375 | 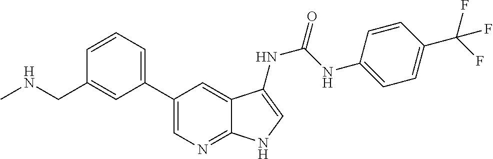 [SMILES: CNCc1cccc(-c2cnc3[nH]cc(NC(=O)Nc4ccc(C(F)(F)F)cc4)c3c2)c1] |
| 376 | 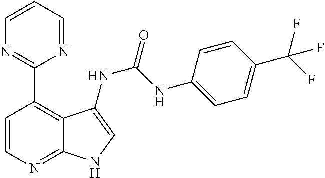 [SMILES: O=C(Nc1ccc(C(F)(F)F)cc1)Nc1c[nH]c2nccc(-c3ncccn3)c12] |
| 377 | 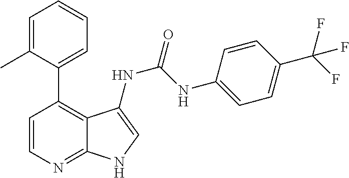 [SMILES: Cc1ccccc1-c1ccnc2[nH]cc(NC(=O)Nc3ccc(C(F)(F)F)cc3)c12] |
| 378 | 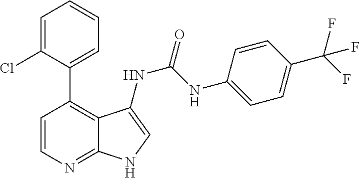 [SMILES: O=C(Nc1ccc(C(F)(F)F)cc1)Nc1c[nH]c2nccc(-c3ccccc3Cl)c12] |
| 379 | 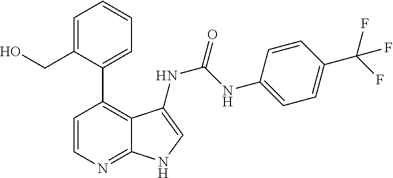 [SMILES: O=C(Nc1ccc(C(F)(F)F)cc1)Nc1c[nH]c2nccc(-c3ccccc3CO)c12] |
| 380 | 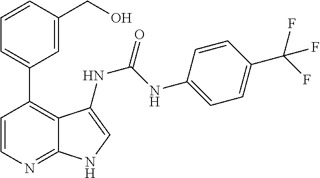 [SMILES: O=C(Nc1ccc(C(F)(F)F)cc1)Nc1c[nH]c2nccc(-c3cccc(CO)c3)c12] |
| 381 | 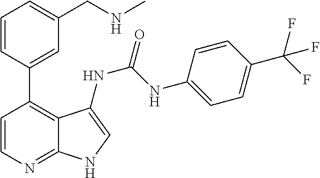 [SMILES: CNCc1cccc(-c2ccnc3[nH]cc(NC(=O)Nc4ccc(C(F)(F)F)cc4)c23)c1] |
| 382 | 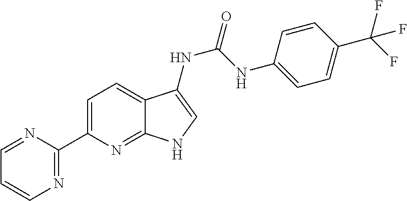 [SMILES: O=C(Nc1ccc(C(F)(F)F)cc1)Nc1c[nH]c2nc(-c3ncccn3)ccc12] |
| 383 | 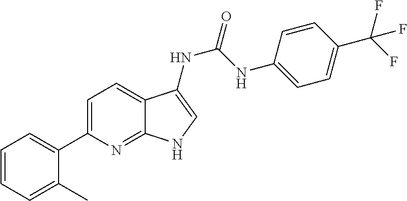 [SMILES: Cc1ccccc1-c1ccc2c(NC(=O)Nc3ccc(C(F)(F)F)cc3)c[nH]c2n1] |
| 384 | 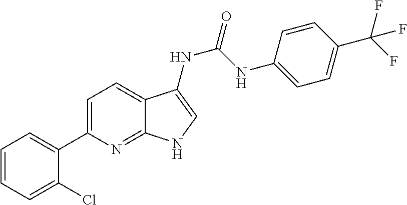 [SMILES: O=C(Nc1ccc(C(F)(F)F)cc1)Nc1c[nH]c2nc(-c3ccccc3Cl)ccc12] |
| 385 | 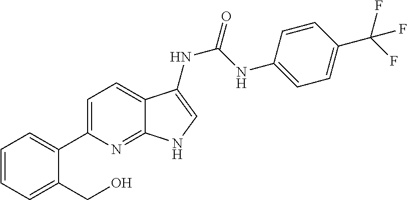 [SMILES: O=C(Nc1ccc(C(F)(F)F)cc1)Nc1c[nH]c2nc(-c3ccccc3CO)ccc12] |
| 386 | 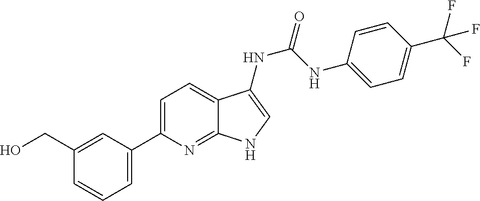 [SMILES: O=C(Nc1ccc(C(F)(F)F)cc1)Nc1c[nH]c2nc(-c3cccc(CO)c3)ccc12] |
| 387 | 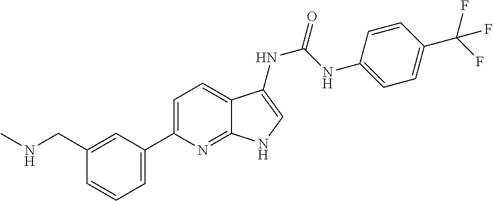 [SMILES: CNCc1cccc(-c2ccc3c(NC(=O)Nc4ccc(C(F)(F)F)cc4)c[nH]c3n2)c1] |
| 388 | 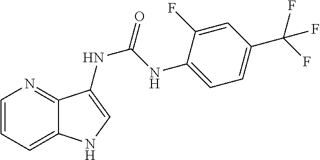 [SMILES: O=C(Nc1ccc(C(F)(F)F)cc1F)Nc1c[nH]c2cccnc12] |
| 389 | 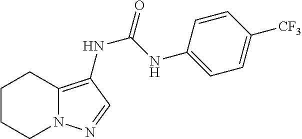 [SMILES: O=C(Nc1ccc(C(F)(F)F)cc1)Nc1cnn2c1CCCC2] |

Compounds of Formula (I) and Table 1, and methods of making and using the same are further described in WO 2020/010092, filed as PCT/US2019/040317 on Jul. 2, 2019; U.S. Provisional 62/693,768, filed on Jul. 3, 2018; and U.S. Provisional 62/861,825, filed on Jun. 14, 2019, each of which is incorporated herein by reference in its entirety.

In one aspect, the STING antagonist is a compound of Formula (II):

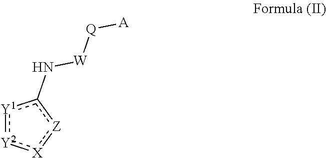

or a pharmaceutically acceptable salt thereof,
wherein:
Z is independently selected from CR¹ and N;
X is independently selected from O, S, N, NR², CR¹, CR³, and NR³;
each [symbol] is a single bond or a double bond provided that the ring including Y¹, Y², X, and Z is heteroaryl;
each of Y¹ and Y² is independently selected from O, S, CR¹, CR³, NR², and N, (in some embodiments, it is provided that when X is other than CR³ or NR³, one of Y¹ and Y² is independently CR³; and when X is CR³ or NR³, both of Y¹ and Y² are other than CR³);

W is selected from the group consisting of: (i) C(═O); (ii) C(═S); (iii) S(O)₁₋₂; (iv) C(═NR^{d}); (v) C(═NH); (vi) C(═C—NO₂); (vii) S(O)N(R^{d}); and (viii) S(O)NH;

Q-A is defined according to (A) or (B) below:

### A

### Q is NH, O, or CH₂, and

### A is:

(i) —(Y^{A1})ₙ—Y^{A2}, wherein:
n is 0 or 1;
Y^{A1} is C₁₋₆ alkylene, which is optionally substituted with from 1-6 R^{a}; and
Y^{A2} is:
   (a) C₃₋₂₀ cycloalkyl, which is optionally substituted with from 1-4 R^{b},
   (b) C₆₋₂₀ aryl, which is optionally substituted with from 1-4 R^{c};
   (c) heteroaryl including from 5-20 ring atoms, wherein from 1-4 ring atoms are heteroatoms, each independently selected from the group consisting of N, N(H), N(R^{d}), O, and S, and wherein one or more of the heteroaryl ring carbon atoms are optionally substituted with from 1-4 independently selected R^{c}, or
   (d) heterocyclyl including from 3-16 ring atoms, wherein from 1-3 ring atoms are heteroatoms, each independently selected from the group consisting of N, N(H), N(R^{d}), and O, and wherein one or more of the heterocyclyl ring carbon atoms are optionally substituted with from 1-4 independently selected R^{b},

### OR

(ii) —Z¹—Z²—Z³, wherein:
Z¹ is C₁₋₃ alkylene, which is optionally substituted with from 1-4 R^{a};
Z² is —N(H)—, —N(R^{d})—, —O—, or —S—; and
Z³ is C₂₋₇ alkyl, which is optionally substituted with from 1-4 R^{a};

### OR

(iii) C₁₋₁₀ alkyl, which is optionally substituted with from 1-6 independently selected R^{a}, or

### B

Q and A, taken together, form:

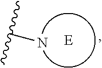

wherein [symbol] denotes point of attachment to W; and

E is heterocyclyl including from 3-16 ring atoms, wherein aside from the nitrogen atom present, from 0-3 additional ring atoms are heteroatoms, each independently selected from the group consisting of N, N(H), N(R^{d}), and O, and wherein one or more of the heterocyclyl ring carbon atoms are optionally substituted with from 1-4 independently selected R^{b},

each R¹ is independently selected from the group consisting of H, halo, cyano, C₁₋₆ alkyl optionally substituted with 1-2 R^{a}, C₂₋₆ alkenyl, C₂₋₆ alkynyl, C₁₋₄ haloalkyl, C₁₋₄ alkoxy, C₁₋₄ haloalkoxy, —S(O)₁₋₂(C₁₋₄ alkyl), —NR^{e}R^{f}, —OH, oxo, —S(O)₁₋₂(NR′R″), —C₁₋₄ thioalkoxy, —NO₂, —C(═O)(C₁₋₄ alkyl), —C(═O)O(C₁₋₄ alkyl), —C(═O)OH, and —C(═O)N(R′)(R″);
R² is selected from the group consisting of:
(i) C₁₋₆ alkyl, which is optionally substituted with from 1-2 independently selected R^{a};
(ii) C₃₋₆ cycloalkyl;
(iii) heterocyclyl including from 3-10 ring atoms, wherein from 1-3 ring atoms are heteroatoms, each independently selected from the group consisting of N, N(H), N(R^{d}), and O.
(iv) —C(O)(C₁₋₄ alkyl);
(v) —C(O)O(C₁₋₄ alkyl);

### (vi) —CON(R′)(R″);

(vii) —S(O)₁₋₂(NR′R″);
(viii) —S(O)₁₋₂(C₁₋₄ alkyl);

### (ix) —OH;

(x) C₁₋₄ alkoxy; and

### (xi) H;

### R³ is:

(i) —(U¹)_{q}—U², wherein:
q is 0 or 1;
U¹ is C₁₋₆ alkylene, which is optionally substituted with from 1-6 R^{a}; and
U² is:
   (a) C₃₋₁₂ cycloalkyl, which is optionally substituted with from 1-4 R^{b},
   (b) C₆₋₁₀ aryl, which is optionally substituted with from 1-4 R^{c};
   (c) heteroaryl including from 5-20 ring atoms, wherein from 1-4 ring atoms are heteroatoms, each independently selected from the group consisting of N, N(H), N(R^{d}), O, and S, and wherein one or more of the heteroaryl ring carbon atoms are optionally substituted with from 1-4 independently selected R^{c}, or
   (d) heterocyclyl including from 3-12 ring atoms, wherein from 1-3 ring atoms are heteroatoms, each independently selected from the group consisting of N, N(H), N(R^{d}), and O, and wherein one or more of the heterocyclyl ring carbon atoms are optionally substituted with from 1-4 independently selected R^{b},

### OR

(ii) C₁₋₁₀ alkyl, which is optionally substituted with from 1-6 independently selected R^{a};

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;

each occurrence of R^{b} is independently selected from the group consisting of: C₁. alkyl optionally substituted with from 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₄ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; C₆₋₁₀ aryl optionally substituted with 1-4 independently selected C₁₋₄ alkyl; and C₃₋₆ cycloalkyl optionally substituted with from 1-4 independently selected C₁₋₄ alkyl;

each occurrence of R^{c} is independently selected from the group consisting of:
(i) halo;
(ii) cyano;
(iii) C₁₋₁₀ alkyl which is optionally substituted with from 1-6 independently selected R^{a};
(iv) C₂₋₆ alkenyl;
(v) C₂₋₆ alkynyl;
(vi) C₁₋₄ haloalkyl;
(vii) C₁₋₄ alkoxy;
(viii) C₁₋₄ haloalkoxy;
(ix) —(C₀₋₃ alkylene)-C₃₋₆ cycloalkyl optionally substituted with from 1-4 independently selected C₁₋₄ alkyl;
(x) —(C₀₋₃ alkylene)-heterocyclyl, wherein the heterocyclyl includes from 3-16 ring atoms, wherein from 1-3 ring atoms are heteroatoms, each independently selected from the group consisting of N, N(H), N(R^{d}), and O;
(xi) —S(O)₁₋₂(C₁₋₄ alkyl);
(xii) —NR^{e}R^{f};
(xiii) —OH;
(xiv) —S(O)₁₋₂(NR′R″);
(xv) —C₁₋₄ thioalkoxy;
(xvi) —NO₂;
(xvii) —C(═O)(C₁₋₄ alkyl);
(xviii) —C(═O)O(C₁₋₄ alkyl);
(xix) —C(═O)OH, and

### (xx) —C(═O)N(R′)(R″);

R^{d} is selected from the group consisting of C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R′ and R″), which are each independently selected from the group consisting of N(R^{d}), O, and S; and

each occurrence of R′ and R″ is independently selected from the group consisting of: H and C₁₋₄ alkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R′ and R″), which are each independently selected from the group consisting of N(R^{d}), O, and S.

In another aspect, the STING antagonist is a compound selected from the group consisting of compounds in Table 2 and pharmaceutically acceptable salts thereof.

TABLE 2

| Com- | |
|---|---|
| pound | |
| # | Structure |
| 1 | 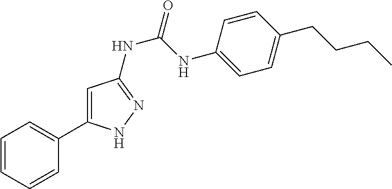 [SMILES: CCCCc1ccc(NC(=O)Nc2cc(-c3ccccc3)[nH]n2)cc1] |
| 2 | 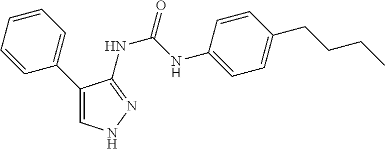 [SMILES: CCCCc1ccc(NC(=O)Nc2n[nH]cc2-c2ccccc2)cc1] |
| 3 | 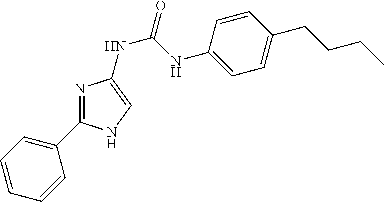 [SMILES: CCCCc1ccc(NC(=O)Nc2c[nH]c(-c3ccccc3)n2)cc1] |
| 4 | 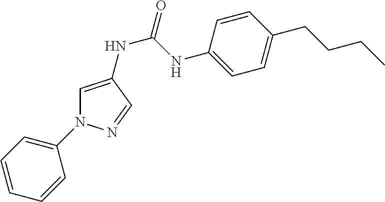 [SMILES: CCCCc1ccc(NC(=O)Nc2cnn(-c3ccccc3)c2)cc1] |
| 5 | 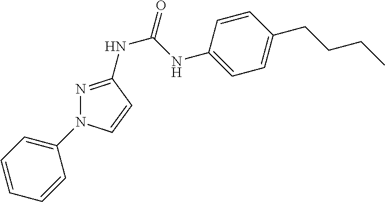 [SMILES: CCCCc1ccc(NC(=O)Nc2ccn(-c3ccccc3)n2)cc1] |
| 6 | 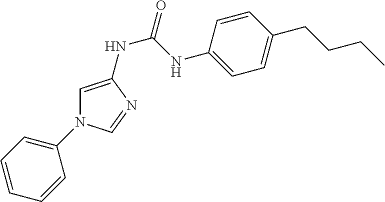 [SMILES: CCCCc1ccc(NC(=O)Nc2cn(-c3ccccc3)cn2)cc1] |
| 7 | 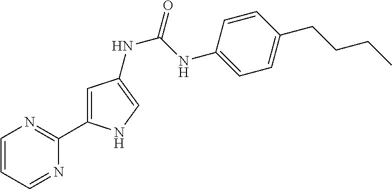 [SMILES: CCCCc1ccc(NC(=O)Nc2c[nH]c(-c3ncccn3)c2)cc1] |
| 8 | 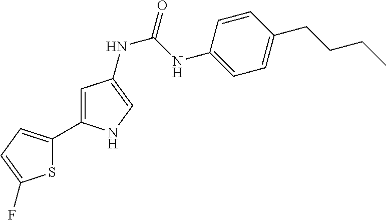 [SMILES: CCCCc1ccc(NC(=O)Nc2c[nH]c(-c3ccc(F)s3)c2)cc1] |
| 9 | 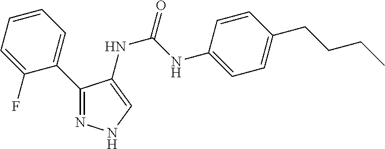 [SMILES: CCCCc1ccc(NC(=O)Nc2c[nH]nc2-c2ccccc2F)cc1] |
| 10 | 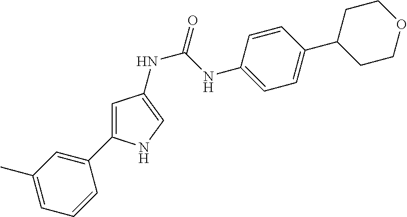 [SMILES: Cc1cccc(-c2cc(NC(=O)Nc3ccc(C4CCOCC4)cc3)c[nH]2)c1] |
| 11 | 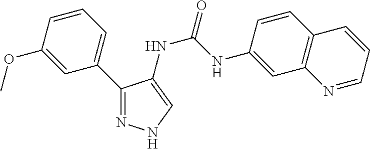 [SMILES: COc1cccc(-c2n[nH]cc2NC(=O)Nc2ccc3cccnc3c2)c1] |
| 12 | 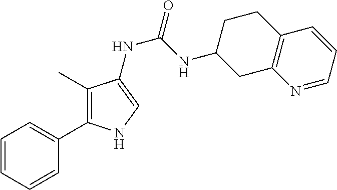 [SMILES: Cc1c(NC(=O)NC2CCc3cccnc3C2)c[nH]c1-c1ccccc1] |
| 13 | 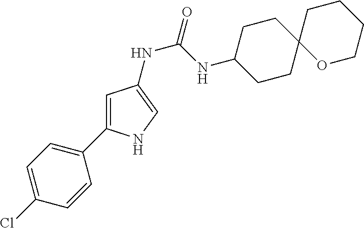 [SMILES: O=C(Nc1c[nH]c(-c2ccc(Cl)cc2)c1)NC1CCC2(CCCCO2)CC1] |
| 14 | 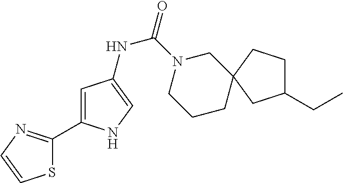 [SMILES: CCC1CCC2(CCCN(C(=O)Nc3c[nH]c(-c4nccs4)c3)C2)C1] |
| 15 | 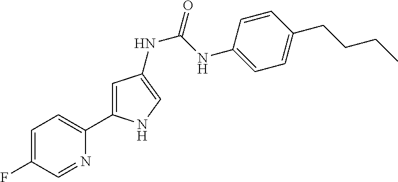 [SMILES: CCCCc1ccc(NC(=O)Nc2c[nH]c(-c3ccc(F)cn3)c2)cc1] |
| 18 | 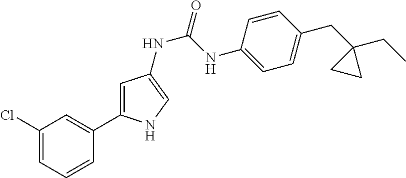 [SMILES: CCC1(Cc2ccc(NC(=O)Nc3c[nH]c(-c4cccc(Cl)c4)c3)cc2)CC1] |
| 19 | 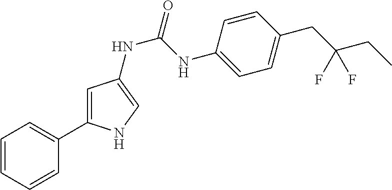 [SMILES: CCC(F)(F)Cc1ccc(NC(=O)Nc2c[nH]c(-c3ccccc3)c2)cc1] |
| 20 | 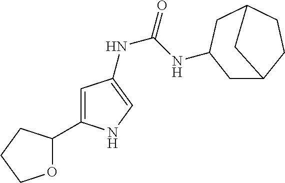 [SMILES: O=C(Nc1c[nH]c(C2CCCO2)c1)NC1CC2CCC(C2)C1] |
| 20a | 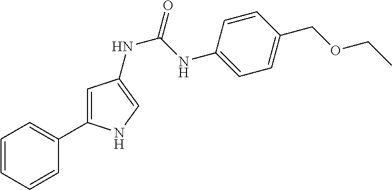 [SMILES: CCOCc1ccc(NC(=O)Nc2c[nH]c(-c3ccccc3)c2)cc1] |
| 21 | 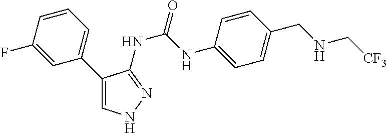 [SMILES: O=C(Nc1ccc(CNCC(F)(F)F)cc1)Nc1n[nH]cc1-c1cccc(F)c1] |
| 22 | 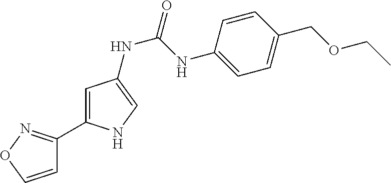 [SMILES: CCOCc1ccc(NC(=O)Nc2c[nH]c(-c3ccon3)c2)cc1] |
| 23 | 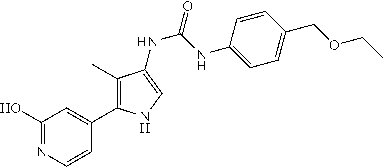 [SMILES: CCOCc1ccc(NC(=O)Nc2c[nH]c(-c3ccnc(O)c3)c2C)cc1] |
| 24 | 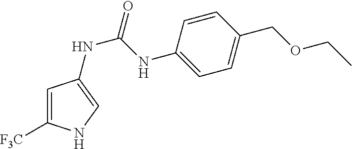 [SMILES: CCOCc1ccc(NC(=O)Nc2c[nH]c(C(F)(F)F)c2)cc1] |
| 25 | 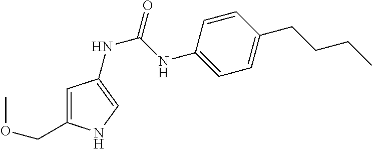 [SMILES: CCCCc1ccc(NC(=O)Nc2c[nH]c(COC)c2)cc1] |
| 26 | 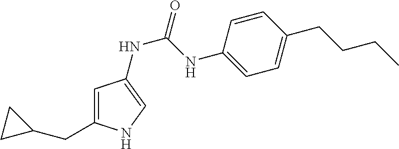 [SMILES: CCCCc1ccc(NC(=O)Nc2c[nH]c(CC3CC3)c2)cc1] |
| 27 | 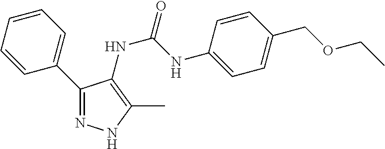 [SMILES: CCOCc1ccc(NC(=O)Nc2c(-c3ccccc3)n[nH]c2C)cc1] |
| 29 | 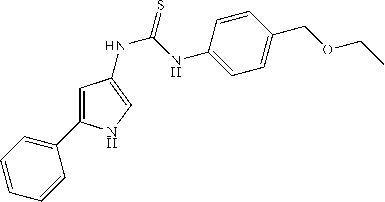 [SMILES: CCOCc1ccc(NC(=S)Nc2c[nH]c(-c3ccccc3)c2)cc1] |
| 30 | 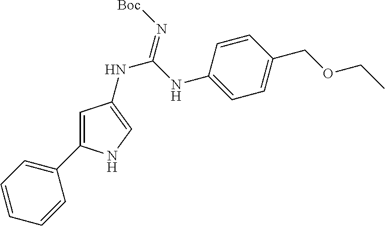 [SMILES: CCOCc1ccc(N/C(=N/C(=O)OC(C)(C)C)Nc2c[nH]c(-c3ccccc3)c2)cc1] |
| 31 | 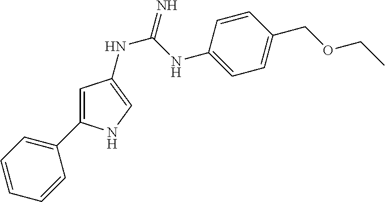 [SMILES: CCOCc1ccc(NC(=N)Nc2c[nH]c(-c3ccccc3)c2)cc1] |
| 20a | 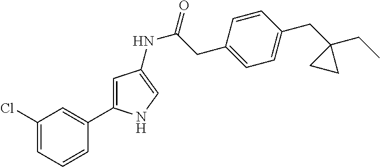 [SMILES: CCC1(Cc2ccc(CC(=O)Nc3c[nH]c(-c4cccc(Cl)c4)c3)cc2)CC1] |
| 20b | 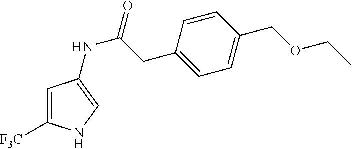 [SMILES: CCOCc1ccc(CC(=O)Nc2c[nH]c(C(F)(F)F)c2)cc1] |
| 32 | 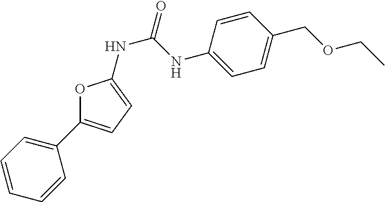 [SMILES: CCOCc1ccc(NC(=O)Nc2ccc(-c3ccccc3)o2)cc1] |
| 33 | 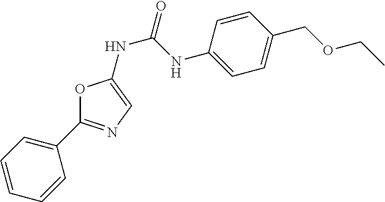 [SMILES: CCOCc1ccc(NC(=O)Nc2cnc(-c3ccccc3)o2)cc1] |
| 34 | 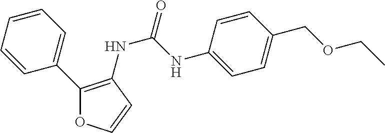 [SMILES: CCOCc1ccc(NC(=O)Nc2ccoc2-c2ccccc2)cc1] |
| 35 | 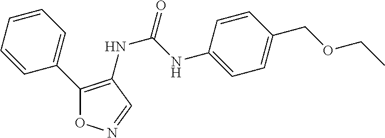 [SMILES: CCOCc1ccc(NC(=O)Nc2cnoc2-c2ccccc2)cc1] |
| 36 | 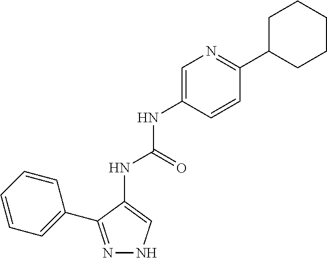 [SMILES: O=C(Nc1ccc(C2CCCCC2)nc1)Nc1c[nH]nc1-c1ccccc1] |
| 37 | 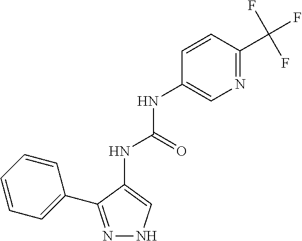 [SMILES: O=C(Nc1ccc(C(F)(F)F)nc1)Nc1c[nH]nc1-c1ccccc1] |
| 38 | 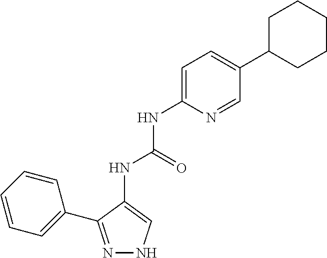 [SMILES: O=C(Nc1ccc(C2CCCCC2)cn1)Nc1c[nH]nc1-c1ccccc1] |
| 39 | 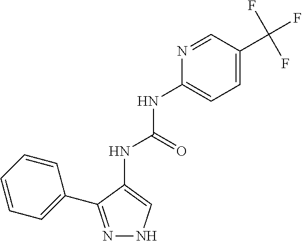 [SMILES: O=C(Nc1ccc(C(F)(F)F)cn1)Nc1c[nH]nc1-c1ccccc1] |
| 40 | 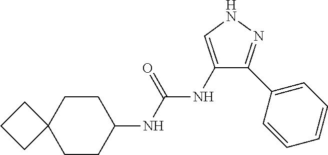 [SMILES: O=C(Nc1c[nH]nc1-c1ccccc1)NC1CCC2(CCC2)CC1] |
| 41 | 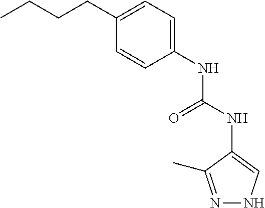 [SMILES: CCCCc1ccc(NC(=O)Nc2c[nH]nc2C)cc1] |
| 42 | 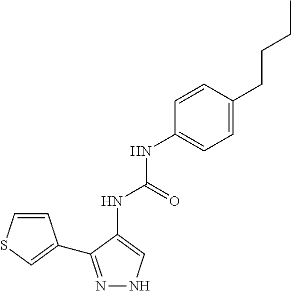 [SMILES: CCCCc1ccc(NC(=O)Nc2c[nH]nc2-c2ccsc2)cc1] |
| 43 | 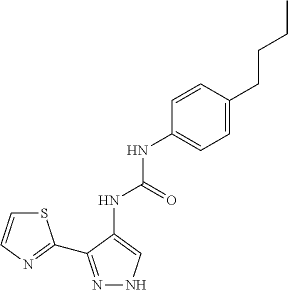 [SMILES: CCCCc1ccc(NC(=O)Nc2c[nH]nc2-c2nccs2)cc1] |
| 44 | 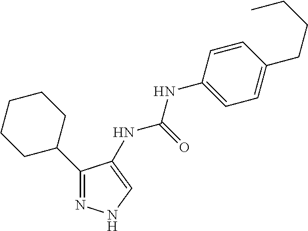 [SMILES: CCCCc1ccc(NC(=O)Nc2c[nH]nc2C2CCCCC2)cc1] |
| 45 | 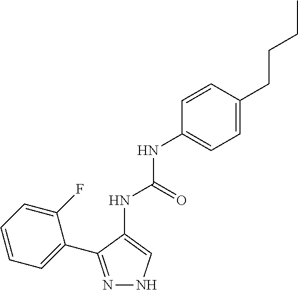 [SMILES: CCCCc1ccc(NC(=O)Nc2c[nH]nc2-c2ccccc2F)cc1] |
| 46 | 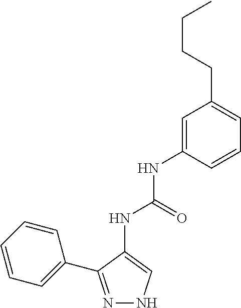 [SMILES: CCCCc1cccc(NC(=O)Nc2c[nH]nc2-c2ccccc2)c1] |
| 47 | 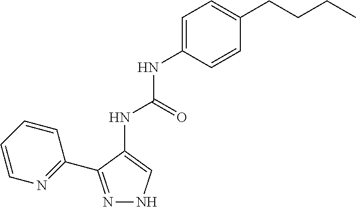 [SMILES: CCCCc1ccc(NC(=O)Nc2c[nH]nc2-c2ccccn2)cc1] |
| 48 | 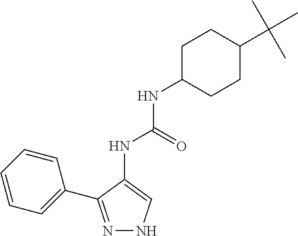 [SMILES: CC(C)(C)C1CCC(NC(=O)Nc2c[nH]nc2-c2ccccc2)CC1] |
| 49 | 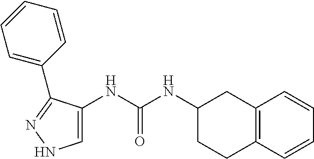 [SMILES: O=C(Nc1c[nH]nc1-c1ccccc1)NC1CCc2ccccc2C1] |
| 50 | 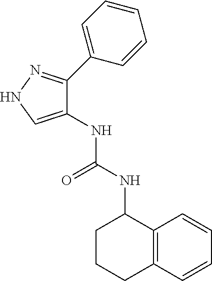 [SMILES: O=C(Nc1c[nH]nc1-c1ccccc1)NC1CCCc2ccccc21] |
| 51 | 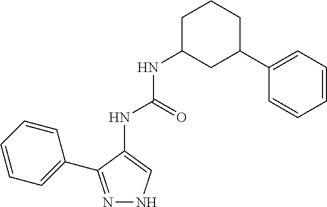 [SMILES: O=C(Nc1c[nH]nc1-c1ccccc1)NC1CCCC(c2ccccc2)C1] |
| 52 | 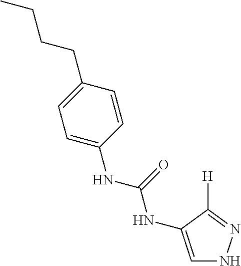 [SMILES: CCCCc1ccc(NC(=O)Nc2cn[nH]c2)cc1] |
| 53 | 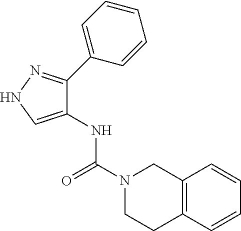 [SMILES: O=C(Nc1c[nH]nc1-c1ccccc1)N1CCc2ccccc2C1] |
| 54 | 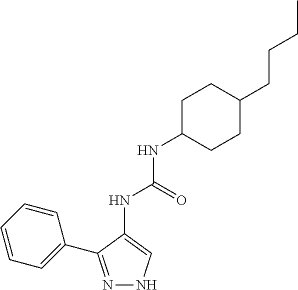 [SMILES: CCCCC1CCC(NC(=O)Nc2c[nH]nc2-c2ccccc2)CC1] |
| 55 | 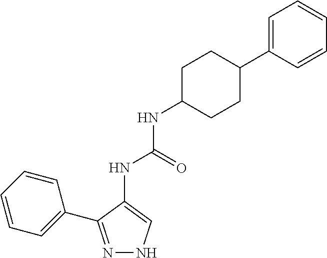 [SMILES: O=C(Nc1c[nH]nc1-c1ccccc1)NC1CCC(c2ccccc2)CC1] |
| 56 | 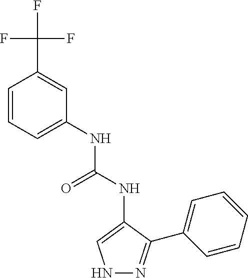 [SMILES: O=C(Nc1cccc(C(F)(F)F)c1)Nc1c[nH]nc1-c1ccccc1] |
| 57 | 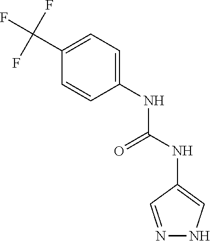 [SMILES: O=C(Nc1ccc(C(F)(F)F)cc1)Nc1cn[nH]c1] |
| 58 | 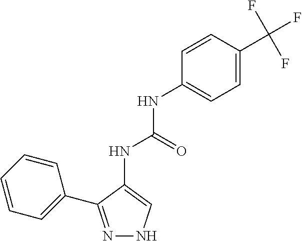 [SMILES: O=C(Nc1ccc(C(F)(F)F)cc1)Nc1c[nH]nc1-c1ccccc1] |
| 59 | 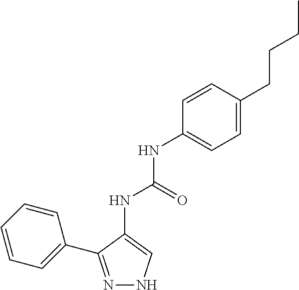 [SMILES: CCCCc1ccc(NC(=O)Nc2c[nH]nc2-c2ccccc2)cc1] |
| 60 | 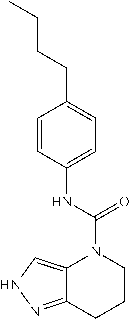 [SMILES: CCCCc1ccc(NC(=O)N2CCCc3n[nH]cc32)cc1] |
| 61 | 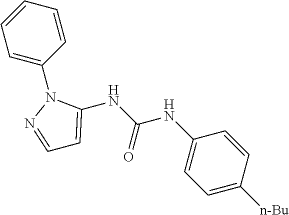 |
| 62 | 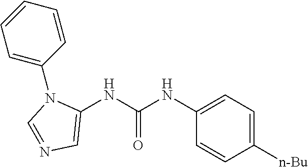 |
| 63 | 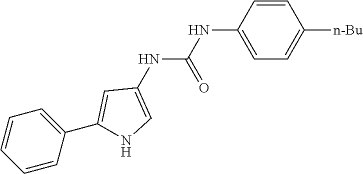 |

Compounds of Formula (II) and Table 2, and methods of making and using the same are further described in WO 2020/010155, filed as PCT/US2019/040418 on Jul. 2, 2019; U.S. Provisional 62/693,878, filed on Jul. 3, 2018; and U.S. Provisional 62/861,078, filed on Jun. 13, 2019, each of which is incorporated herein by reference in its entirety.

In one aspect, the STING antagonist is a compound of Formula (III):

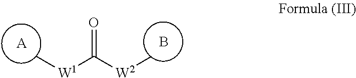

or a pharmaceutically acceptable salt thereof or a tautomer thereof,

wherein:

one of W¹ and W² is —N(H)—, —N(R^{d})— (e.g., —N(H)— or —N(C₁₋₃ alkyl)-), —N(H)—(W²)—, or —N(R^{d})—(W¹²)—,

the other one of W¹ and W² is a bond, —O—, —O—(W¹²)—, or C₁-C₆ alkylene optionally substituted with from 1-3 R^{a} (e.g., C₁-C₃, e.g., CH₂, CHR^{a}, or CR^{a}₂); wherein W¹² is C₁-C₆ alkylene optionally substituted with from 1-3 R^{a},

provided the one of W¹ and W² is attached to the C(═O) moiety of Formula III through a nitrogen atom;

A is selected from the group consisting of (A-1), (A-2), and (A-3):

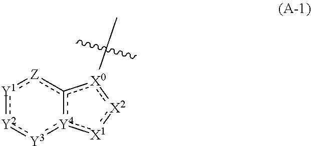

wherein

Z is selected from the group consisting of: a bond, CH, CR¹, CR³, N, NH, N(R¹) and N(R²);

each of Y¹, Y², and Y³ is independently selected from the group consisting of O, S, CH, CR¹, CR³, N, NH, N(R′), and NR²;

Y⁴ is C or N;

X⁰ is C or N;

X¹ is selected from the group consisting of O, S, N, NH, NR¹, NR², CH, CR¹, and CR³;

X² is selected from the group consisting of O, S, N, NH, NR¹, NR², CH, CR¹, and CR³; and

each [symbol] is independently a single bond or a double bond, provided that the five-membered ring comprising Y⁴, X⁰, X¹, and X² is heteroaryl; and

the ring comprising Z, Y¹, Y², Y³, and Y⁴ is aromatic (i.e., carbocyclic aromatic or heteroaromatic);

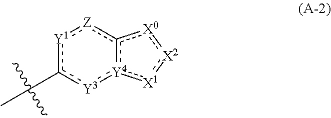

wherein:

Z is selected from the group consisting of:

a bond, CH, CR¹, CR³, N, NH, N(R¹) and N(R²);

each of Y¹ and Y³ is independently selected from the group consisting of O, S, CH, CR¹, CR³, N, NH, N(R′), and NR²;

Y⁴ is C or N;

X⁰ is selected from the group consisting of O, S, N, NH, NR¹, NR², CH, CR¹, and CR³;

X¹ is selected from the group consisting of O, S, N, NH, NR¹, NR², CH, CR¹, and CR³;

X² is selected from the group consisting of O, S, N, NH, NR¹, NR², CH, CR¹, and CR³; and

each [symbol] is independently a single bond or a double bond, provided that the five-membered ring comprising Y⁴, X¹, and X² is heteroaryl; and

the ring comprising Z, Y¹, Y³, and Y⁴ is aromatic (i.e., carbocyclic aromatic or heteroaromatic);

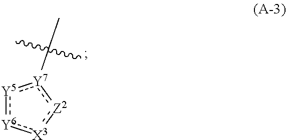

wherein:

Y⁷ is N or C;

Z² is selected from CH, CR², and N;

X³ is selected from O, S, N, NH, NR¹, NR², CH, CR¹, and CR³;

each of Y⁵ and Y⁶ is independently selected from O, S, CH, CR¹, CR³, NR¹, NR², NH, and N; and

each [symbol] is independently a single bond or a double bond, provided that the five-membered ring comprising Y⁵, Y⁶, Y⁷, X³, and Z² is heteroaromatic, and

further provided that:

when X³ is NR¹ or CR¹, then each of Y⁵ and Y⁶ is independently selected from O, S, CH, CR³, NR², NH, and N; and

when X³ is selected from O, S, N, NH, NR², CH, and CR³, then one of Y⁵ and Y⁶ is CR¹ or NR¹;

### B is:

(a) C₁₋₁₅ alkyl which is optionally substituted with from 1-6 independently selected R^{a};

(b) C₃₋₂₀ cycloalkyl, which is optionally substituted with from 1-4 R^{b};

(c) phenyl substituted with from 1-4 R^{c};

(d) C₈₋₂₀ aryl optionally substituted with from 1-4 R^{c};

(e) heteroaryl including from 5-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; or

(f) heterocyclyl including from 3-16 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b};

### R¹ is:

(i) —(U¹)_{q}—U², wherein:
q is 0 or 1;
U¹ is C₁₋₆ alkylene, which is optionally substituted with from 1-6 R^{a}; and
U² is:
   (a) C₃₋₁₂ cycloalkyl, which is optionally substituted with from 1-4 R^{b},
   (b) C₆₋₁₀ aryl, which is optionally substituted with from 1-4 R^{c};
   (c) heteroaryl including from 5-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}, or
   (d) heterocyclyl including from 3-12 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},

OR

(ii) C₁₋₁₀ alkyl, which is optionally substituted with from 1-6 independently selected R^{a};

each occurrence of R² is independently selected from the group consisting of:

(i) C₁₋₆ alkyl, which is optionally substituted with from 1-4 independently selected R^{a};

(ii) C₃₋₆ cycloalkyl;

(iii) heterocyclyl including from 3-10 ring atoms, wherein from 1-3 ring atoms are heteroatoms, each independently selected from the group consisting of N, N(H), N(R^{d}), O, and S(O)₀₋₂;

(iv) —C(O)(C₁₋₄ alkyl);

(v) —C(O)O(C₁₋₄ alkyl);

(vi) —CON(R′)(R″);

(vii) —S(O)₁₋₂(NR′R″);

(viii) —S(O)₁₋₂(C₁₋₄ alkyl);

(ix) —OH; and

(x) C₁₋₄ alkoxy;

each occurrence of R³ is independently selected from the group consisting of halo, cyano, C₂₋₆ alkenyl, C₂₋₆ alkynyl, C₁₋₄ alkoxy optionally substituted with C₃₋₆ cycloalkyl, C₁₋₄ haloalkoxy, —S(O)₁₋₂(C₁₋₄ alkyl), —NR^{e}R^{f}, —OH, oxo, —S(O)₁₋₂(NR′R″), —C₁₋₄ thioalkoxy, —NO₂, —C(═O)(C₁₋₄ alkyl), —C(═O)O(C₁₋₄ alkyl), —C(═O)OH, and —C(═O)N(R′)(R″);

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;

each occurrence of R^{b} is independently selected from the group consisting of: C₁. alkyl optionally substituted with from 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₄ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{e} is independently selected from the group consisting of: (a) halo; (b) cyano; (c) C₁₋₅ alkyl which is optionally substituted with from 1-6 independently selected R^{a}; (d) C₂₋₆ alkenyl; (e) C₂₋₆ alkynyl; (g) C₁₋₄ alkoxy optionally substituted with C₁₋₄ alkoxy; (h) C₁₋₄ haloalkoxy; (i) —S(O)₁₋₂(C₁₋₄ alkyl); (j) —NR^{e}R^{f}; (k) —OH; (l) —S(O)₁₋₂(NR′R″); (m) —C₁₋₄ thioalkoxy optionally substituted with from 1-4 halo;

(n) —NO₂; (o) —C(═O)(C₁₋₄ alkyl); (p) —C(═O)O(C₁₋₄ alkyl); (q) —C(═O)OH; (r) —C(═O)N(R′)(R″); and (s) -L¹-L²-R^{h};

R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene;

-L² is —O—, —N(H)—, —S—, or a bond;

R^{h} is selected from:
C₃₋₈ cycloalkyl optionally substituted with from 1-4 substituents independently selected from the group consisting of halo, C₁₋₄ alkyl, and C₁₋₄ haloalkyl (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo, C₁₋₄ alkyl, and C₁₋₄ haloalkyl;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo, C₁. 4 alkyl, and C₁₋₄ haloalkyl; and
C₆₋₁₀ aryl, which is optionally substituted with from 1-4 substituents independently selected from the group consisting of halo, C₁₋₄ alkyl, or C₁₋₄ haloalkyl; and
   each occurrence of R′ and R″ is independently selected from the group consisting of: H, C₁₋₄ alkyl, and C₆₋₁₀ aryl optionally substituted with from 1-2 substituents selected from halo, C₁₋₄ alkyl, and C₁₋₄ haloalkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(R^{d}), O, and S.

In some embodiments of the compound of Formula (III), A is (A-1).

In some embodiments, A is:

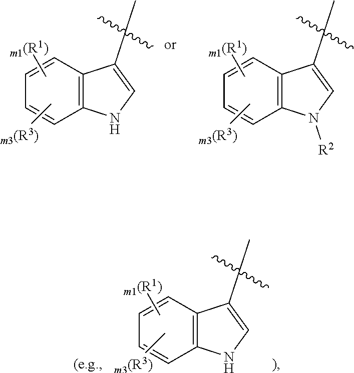

wherein m1=0, 1, 2, or 3; and m3=0, 1, 2, or 3 (e.g., m1=0 or 1; and m3=0, 1, or 2). For example, m1 can be 0; and m3 can be 2; or m1 can be 1; and m3 can be 0; or m1 can be 0; and m3 can be 0.

In some embodiments of the compound of Formula (III), W¹ is —NH—. In some embodiments of the compound of Formula (III), W² is a bond. In some embodiments of the compound of Formula (III), B is phenyl substituted with from 1-4 R^{c}.

In another aspect, the STING antagonist is a compound of Formula (IV):

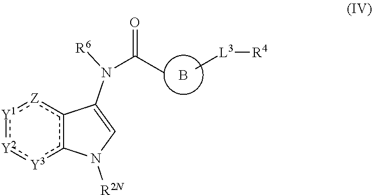

or a pharmaceutically acceptable salt thereof or a tautomer thereof, wherein:

Z is selected from the group consisting of: CH, CR¹, CR³, N, NH, N(R¹) and N(R²);

each of Y¹, Y², and Y³ is independently selected from the group consisting of CH, CR¹, CR³, N, NH, N(R¹), and NR²;

each [symbol] is independently a single bond or a double bond, provided that:

the 6-membered ring comprising Z, Y¹, Y², and Y³ is aromatic;

provided that Y³ cannot be N when each of each of Y¹, Y², and Y³ is independently selected from the group consisting of CH, CR¹, CR³; and

when each of Z, Y¹, Y², and Y³ is independently selected from the group consisting of CH, CR¹, and CR³, from 1-4 of Z, Y¹, Y², and Y³ is selected from the group consisting of CR¹ and CR³;

R^{2N} is H or R²;

R⁶ is selected from the group consisting of H and R^{d};

B is a monocyclic heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-2 independently selected R^{c};

-L³ is a bond or C₁₋₃ alkylene;

R⁴ is selected from the group consisting of:

(a) C₃₋₁₂ cycloalkyl, which is optionally substituted with from 1-4 independently selected R^{4′},

(b) heterocyclyl including from 3-12 ring atoms, wherein from 1-3 ring atoms are heteroatoms, each independently selected from the group consisting of N, N(H), N(R^{d}), O, and S(O)₀₋₂, and wherein one or more ring carbon atoms of the heterocyclyl is optionally substituted with from 1-4 independently selected R^{4′};

(c) heteroaryl including from 5-12 ring atoms, wherein from 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with from 1-4 independently selected R^{4′}; and

(d) C₆₋₁₀ aryl optionally substituted with from 1-4 independently selected R^{4′};
wherein each R^{4′} is independently selected from the group consisting of: halo; —CN; —NO₂; —OH; —C₁₋₄ alkyl optionally substituted with from 1-2 independently selected R^{a}; —C₂₋₄ alkenyl; —C₂₋₄ alkynyl; —C₁₋₄ haloalkyl; —C₁₋₆ alkoxy optionally substituted with from 1-2 independently selected R^{a}; —C₁₋₆ haloalkoxy; S(O)₁₋₂(C₁₋₄ alkyl); —NR′R″; oxo; —S(O)₁₋₂(NR′R″); —C₁₋₄ thioalkoxy; —C(═O)(C₁₋₄ alkyl); —C(═O)O(C₁₋₄ alkyl); —C(═O)OH; and —C(═O)N(R′)(R″);

R¹ is:

(i) —(U¹)_{q}—U², wherein:
q is 0 or 1;
U¹ is C₁₋₆ alkylene, which is optionally substituted with from 1-6 R^{a}; and
U² is:

(a) C₃₋₁₂ cycloalkyl, which is optionally substituted with from 1-4 R^{b},

(b) C₆₋₁₀ aryl, which is optionally substituted with from 1-4 R^{c};

(c) heteroaryl including from 5-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}, or

(d) heterocyclyl including from 3-12 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},

OR

(ii) C₁₋₁₀ alkyl, which is optionally substituted with from 1-6 independently selected R^{a};

each occurrence of R² is independently selected from the group consisting of:

(i) C₁₋₆ alkyl, which is optionally substituted with from 1-4 independently selected R^{a};

(ii) C₃₋₆ cycloalkyl;

(iii) heterocyclyl including from 3-10 ring atoms, wherein from 1-3 ring atoms are heteroatoms, each independently selected from the group consisting of N, N(H), N(R^{d}), O, and S(O)₀₋₂;

(iv) —C(O)(C₁₋₄ alkyl);

(v) —C(O)O(C₁₋₄ alkyl);

(vi) —CON(R′)(R″);

(vii) —S(O)₁₋₂(NR′R″);

(viii) —S(O)₁₋₂(C₁₋₄ alkyl);

(ix) —OH; and

(x) C₁₋₄ alkoxy;

each occurrence of R³ is independently selected from the group consisting of halo, cyano, C₂₋₆ alkenyl, C₂₋₆ alkynyl, C₁₋₄ alkoxy optionally substituted with C₃₋₆ cycloalkyl, C₁₋₄ haloalkoxy, —S(O)₁₋₂(C₁₋₄ alkyl), —NR^{e}R^{f}, —OH, oxo, —S(O)₁₋₂(NR′R″), —C₁₋₄ thioalkoxy, —NO₂, —C(═O)(C₁₋₄ alkyl), —C(═O)O(C₁₋₄ alkyl), —C(═O)OH, and —C(═O)N(R′)(R″);

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;

each occurrence of R^{b} is independently selected from the group consisting of: C₁. alkyl optionally substituted with from 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₄ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{e} is independently selected from the group consisting of:

(a) halo; (b) cyano; (c) C₁₋₁₅ alkyl which is optionally substituted with from 1-6 independently selected R^{a}; (d) C₂₋₆ alkenyl; (e) C₂₋₆ alkynyl; (g) C₁₋₄ alkoxy optionally substituted with C₁₋₄ alkoxy; (h) C₁₋₄ haloalkoxy; (i) —S(O)₁₋₂(C₁₋₄ alkyl); (j) —NR^{e}R^{f}; (k) —OH; (l) —S(O)₁₋₂(NR′R″); (m) —C₁₋₄ thioalkoxy optionally substituted with from 1-4 halo; (n) —NO₂; (o) —C(═O)(C₁₋₄ alkyl); (p) —C(═O)O(C₁₋₄ alkyl); (q) —C(═O)OH; (r) —C(═O)N(R′)(R″); and (s) -L¹-L²-R^{h};

R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene;

-L² is —O—, —N(H)—, —S—, or a bond;

R^{h} is selected from:
C₃₋₈ cycloalkyl optionally substituted with from 1-4 substituents independently selected from the group consisting of halo, C₁₋₄ alkyl, and C₁₋₄ haloalkyl (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo, C₁₋₄ alkyl, and C₁₋₄ haloalkyl;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo, C₁. 4 alkyl, and C₁₋₄ haloalkyl; and
C₆₋₁₀ aryl, which is optionally substituted with from 1-4 substituents independently selected from the group consisting of halo, C₁₋₄ alkyl, or C₁₋₄ haloalkyl; and

each occurrence of R′ and R″ is independently selected from the group consisting of: H, C₁₋₄ alkyl, and C₆₋₁₀ aryl optionally substituted with from 1-2 substituents selected from halo, C₁₋₄ alkyl, and C₁₋₄ haloalkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(R^{d}), O, and S.

In some embodiments of the compound of Formula (IV), each of Z, Y¹, Y², and Y³ is independently selected from the group consisting of: CH, CR¹, CR³, and N. For example, each of Z, Y¹, Y², and Y³ is independently selected from the group consisting of: CH, CR¹, and CR³.

In some embodiments of the compound of Formula (IV), the compound has a formula selected from the group consisting of:

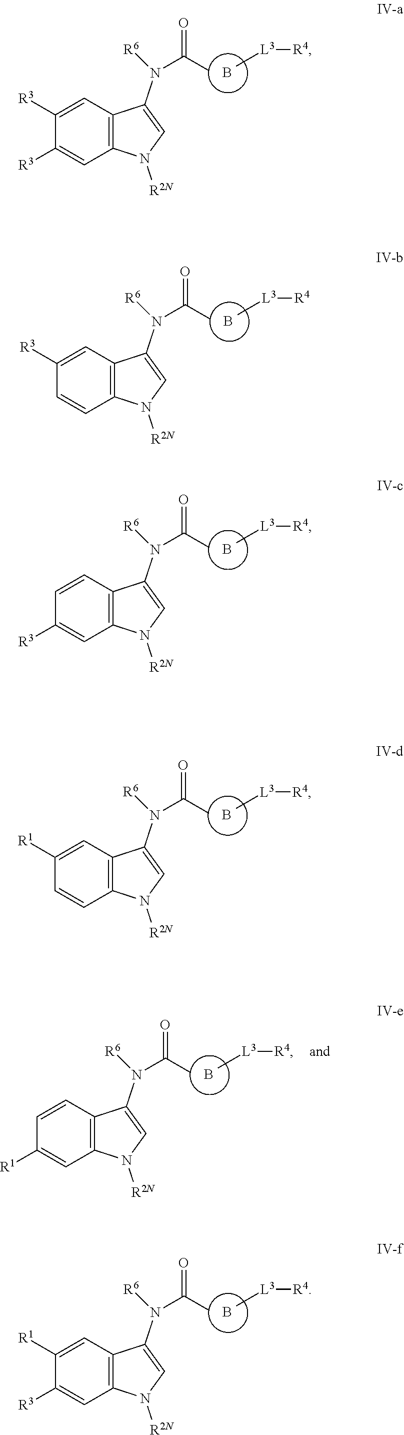

In some embodiments of the compound of Formula (IV), the compound has a formula selected from the group consisting of:

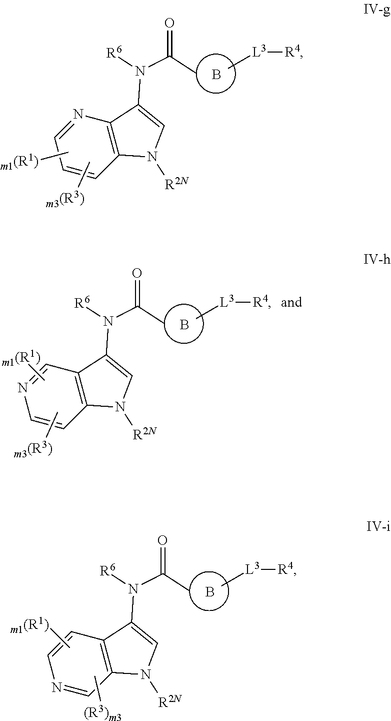

wherein m1 is 0 or 1; and m3 is 0, 1, or 2.

In some embodiments of the compound of Formula (IV), R^{2N} is H.

In another aspect, the STING antagonist is a compound selected from the group consisting of compounds in Table 3 and pharmaceutically acceptable salts thereof.

TABLE 3

| Compound | |
|---|---|
| No. | Structure |
| 1 | 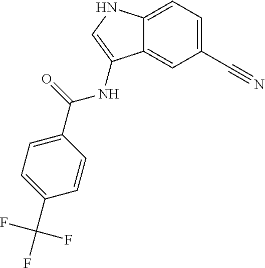 [SMILES: N#Cc1ccc2[nH]cc(NC(=O)c3ccc(C(F)(F)F)cc3)c2c1] |
| 2 | 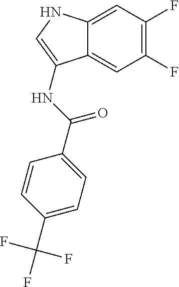 [SMILES: O=C(Nc1c[nH]c2cc(F)c(F)cc12)c1ccc(C(F)(F)F)cc1] |
| 3 | 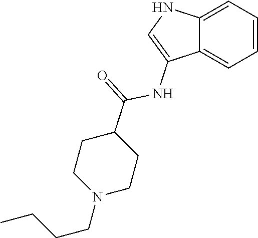 [SMILES: CCCCN1CCC(C(=O)Nc2c[nH]c3ccccc23)CC1] |
| 4 | 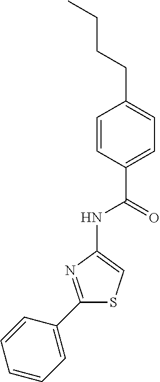 [SMILES: CCCCc1ccc(C(=O)Nc2csc(-c3ccccc3)n2)cc1] |
| 5 | 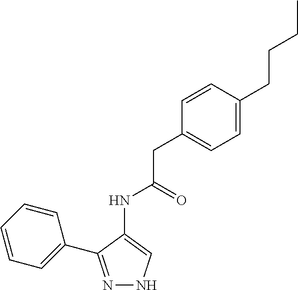 [SMILES: CCCCc1ccc(CC(=O)Nc2c[nH]nc2-c2ccccc2)cc1] |
| 6 | 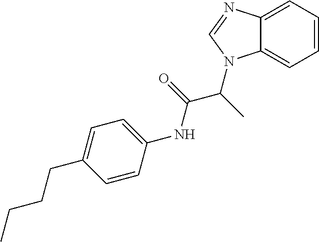 [SMILES: CCCCc1ccc(NC(=O)C(C)n2cnc3ccccc32)cc1] |
| 7 | 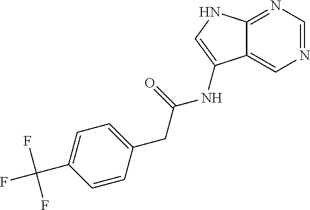 [SMILES: O=C(Cc1ccc(C(F)(F)F)cc1)Nc1c[nH]c2ncncc12] |
| 8 | 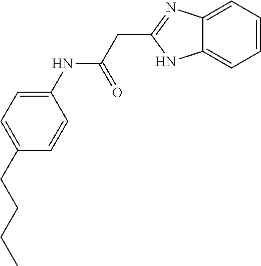 [SMILES: CCCCc1ccc(NC(=O)Cc2nc3ccccc3[nH]2)cc1] |
| 9 | 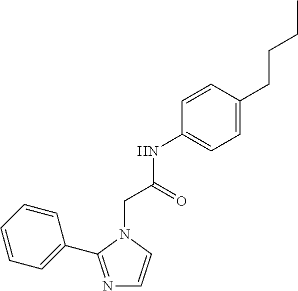 [SMILES: CCCCc1ccc(NC(=O)Cn2ccnc2-c2ccccc2)cc1] |
| 10 | 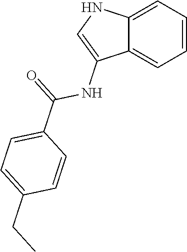 [SMILES: CCc1ccc(C(=O)Nc2c[nH]c3ccccc23)cc1] |
| 11 | 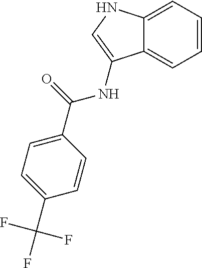 [SMILES: O=C(Nc1c[nH]c2ccccc12)c1ccc(C(F)(F)F)cc1] |
| 12 | 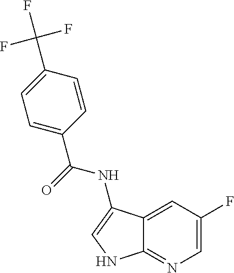 [SMILES: O=C(Nc1c[nH]c2ncc(F)cc12)c1ccc(C(F)(F)F)cc1] |
| 13 | 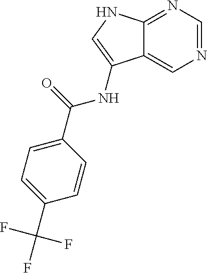 [SMILES: O=C(Nc1c[nH]c2ncncc12)c1ccc(C(F)(F)F)cc1] |
| 14 | 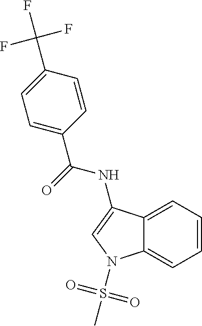 [SMILES: CS(=O)(=O)n1cc(NC(=O)c2ccc(C(F)(F)F)cc2)c2ccccc21] |
| 15 | 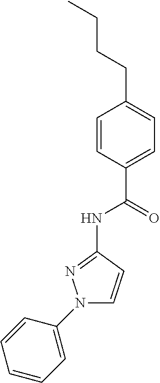 [SMILES: CCCCc1ccc(C(=O)Nc2ccn(-c3ccccc3)n2)cc1] |
| 16 | 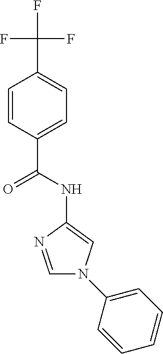 [SMILES: O=C(Nc1cn(-c2ccccc2)cn1)c1ccc(C(F)(F)F)cc1] |
| 17 | 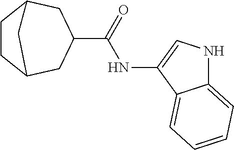 [SMILES: O=C(Nc1c[nH]c2ccccc12)C1CC2CCC(C2)C1] |
| 18 | 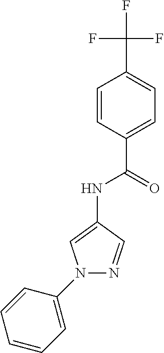 [SMILES: O=C(Nc1cnn(-c2ccccc2)c1)c1ccc(C(F)(F)F)cc1] |
| 19 | 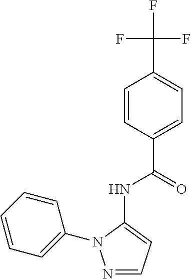 [SMILES: O=C(Nc1ccnn1-c1ccccc1)c1ccc(C(F)(F)F)cc1] |
| 20 | 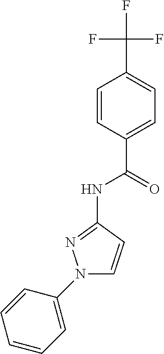 [SMILES: O=C(Nc1ccn(-c2ccccc2)n1)c1ccc(C(F)(F)F)cc1] |
| 21 | 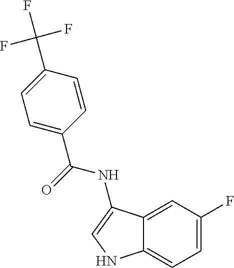 [SMILES: O=C(Nc1c[nH]c2ccc(F)cc12)c1ccc(C(F)(F)F)cc1] |
| 22 | 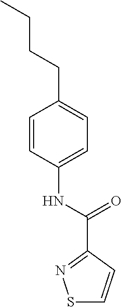 [SMILES: CCCCc1ccc(NC(=O)c2ccsn2)cc1] |
| 23 | 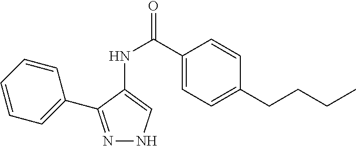 [SMILES: CCCCc1ccc(C(=O)Nc2c[nH]nc2-c2ccccc2)cc1] |
| 24 | 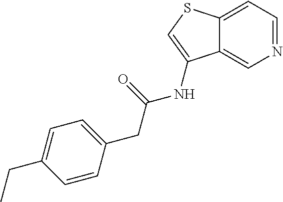 [SMILES: CCc1ccc(CC(=O)Nc2csc3ccncc23)cc1] |
| 25 | 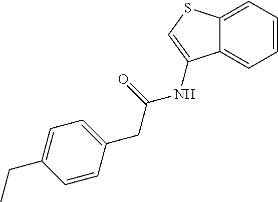 [SMILES: CCc1ccc(CC(=O)Nc2csc3ccccc23)cc1] |
| 26 | 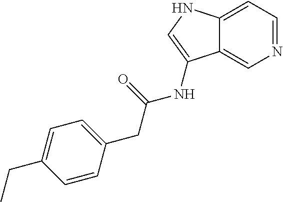 [SMILES: CCc1ccc(CC(=O)Nc2c[nH]c3ccncc23)cc1] |
| 27 | 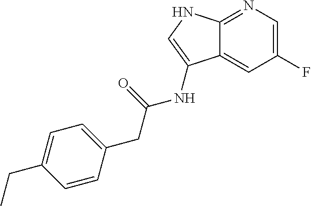 [SMILES: CCc1ccc(CC(=O)Nc2c[nH]c3ncc(F)cc23)cc1] |
| 28 | 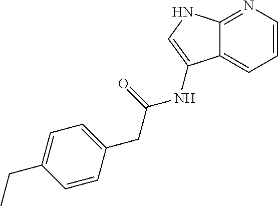 [SMILES: CCc1ccc(CC(=O)Nc2c[nH]c3ncccc23)cc1] |
| 29 | 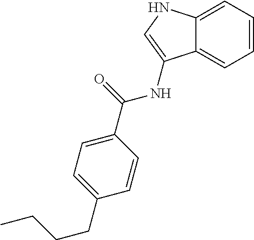 [SMILES: CCCCc1ccc(C(=O)Nc2c[nH]c3ccccc23)cc1] |
| 30 | 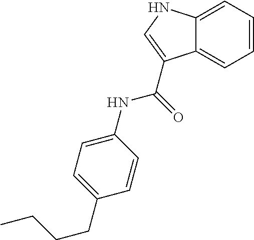 [SMILES: CCCCc1ccc(NC(=O)c2c[nH]c3ccccc23)cc1] |
| 31 | 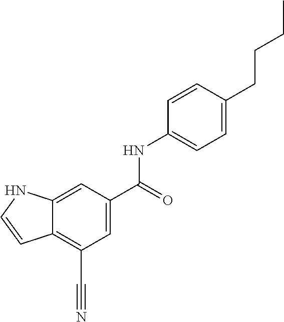 [SMILES: CCCCc1ccc(NC(=O)c2cc(C#N)c3cc[nH]c3c2)cc1] |
| 32 | 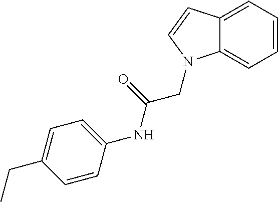 [SMILES: CCc1ccc(NC(=O)Cn2ccc3ccccc32)cc1] |
| 33 | 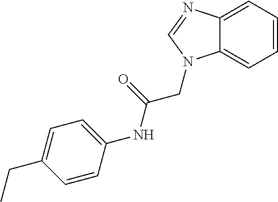 [SMILES: CCc1ccc(NC(=O)Cn2cnc3ccccc32)cc1] |
| 34 | 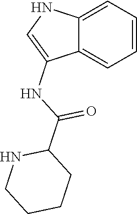 [SMILES: O=C(Nc1c[nH]c2ccccc12)C1CCCCN1] |
| 35 | 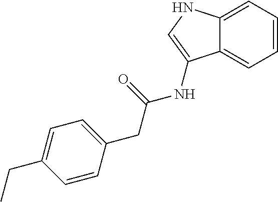 [SMILES: CCc1ccc(CC(=O)Nc2c[nH]c3ccccc23)cc1] |
| 36 | 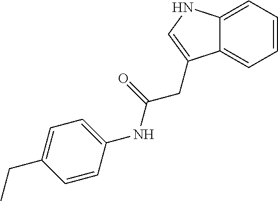 [SMILES: CCc1ccc(NC(=O)Cc2c[nH]c3ccccc23)cc1] |
| 37 | 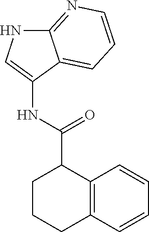 [SMILES: O=C(Nc1c[nH]c2ncccc12)C1CCCc2ccccc21] |
| 38 | 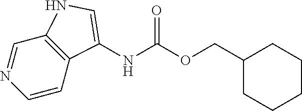 [SMILES: O=C(Nc1c[nH]c2cnccc12)OCC1CCCCC1] |
| 39 | 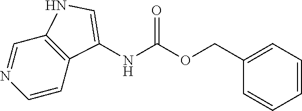 [SMILES: O=C(Nc1c[nH]c2cnccc12)OCc1ccccc1] |
| 40 | 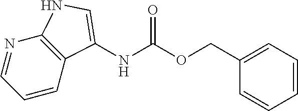 [SMILES: O=C(Nc1c[nH]c2ncccc12)OCc1ccccc1] |
| 41 | 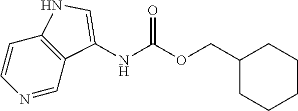 [SMILES: O=C(Nc1c[nH]c2ccncc12)OCC1CCCCC1] |
| 42 | 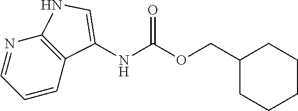 [SMILES: O=C(Nc1c[nH]c2ncccc12)OCC1CCCCC1] |
| 43 | 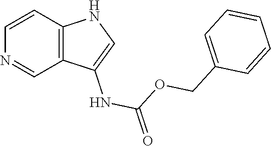 [SMILES: O=C(Nc1c[nH]c2ccncc12)OCc1ccccc1] |
| 44 | 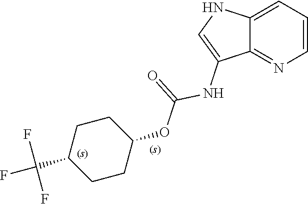 [SMILES: O=C(Nc1c[nH]c2cccnc12)O[C@H]1CC[C@@H](C(F)(F)F)CC1] |
| 45 | 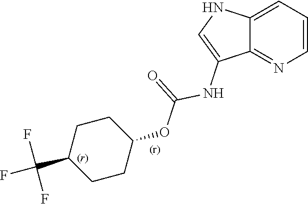 [SMILES: O=C(Nc1c[nH]c2cccnc12)O[C@H]1CC[C@H](C(F)(F)F)CC1] |
| 46 | 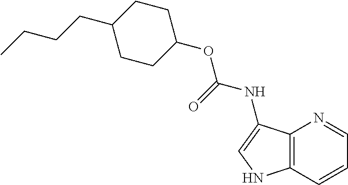 [SMILES: CCCCC1CCC(OC(=O)Nc2c[nH]c3cccnc23)CC1] |
| 47 | 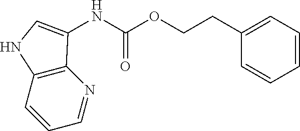 [SMILES: O=C(Nc1c[nH]c2cccnc12)OCCc1ccccc1] |
| 48 | 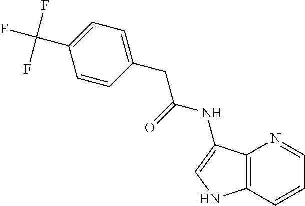 [SMILES: O=C(Cc1ccc(C(F)(F)F)cc1)Nc1c[nH]c2cccnc12] |
| 49 | 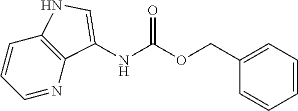 [SMILES: O=C(Nc1c[nH]c2cccnc12)OCc1ccccc1] |
| 50 | 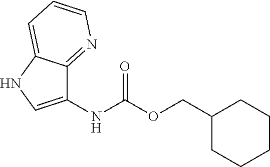 [SMILES: O=C(Nc1c[nH]c2cccnc12)OCC1CCCCC1] |
| 51 | 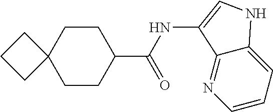 [SMILES: O=C(Nc1c[nH]c2cccnc12)C1CCC2(CCC2)CC1] |
| 52 | 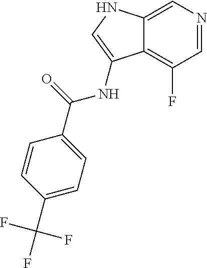 [SMILES: O=C(Nc1c[nH]c2cncc(F)c12)c1ccc(C(F)(F)F)cc1] |
| 53 | 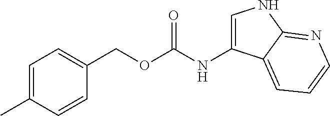 [SMILES: Cc1ccc(COC(=O)Nc2c[nH]c3ncccc23)cc1] |
| 54 | 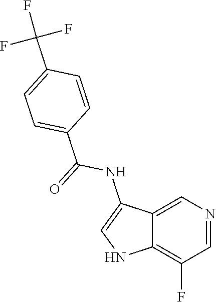 [SMILES: O=C(Nc1c[nH]c2c(F)cncc12)c1ccc(C(F)(F)F)cc1] |
| 55 | 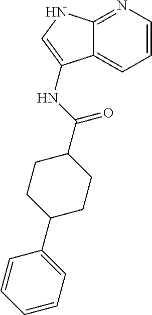 [SMILES: O=C(Nc1c[nH]c2ncccc12)C1CCC(c2ccccc2)CC1] |
| 56 | 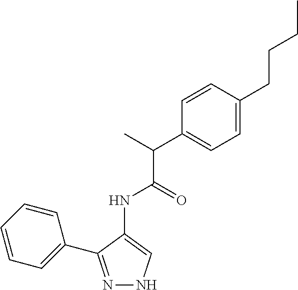 [SMILES: CCCCc1ccc(C(C)C(=O)Nc2c[nH]nc2-c2ccccc2)cc1] |
| 57 | 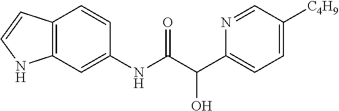 |
| 58 | 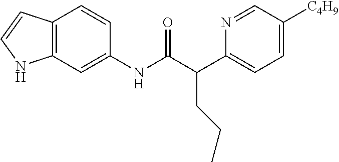 |
| 59 | 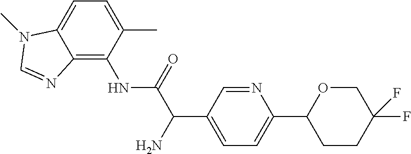 [SMILES: Cc1ccc2c(ncn2C)c1NC(=O)C(N)c1ccc(C2CCC(F)(F)CO2)nc1] |
| 60 | 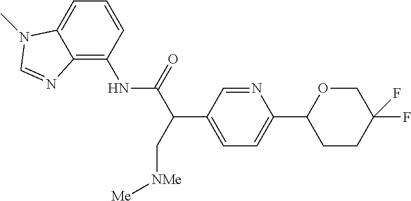 [SMILES: CN(C)CC(C(=O)Nc1cccc2c1ncn2C)c1ccc(C2CCC(F)(F)CO2)nc1] |
| 61 | 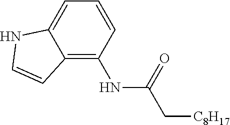 |
| 62 | 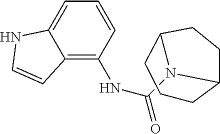 [SMILES: O=C(Nc1cccc2[nH]ccc12)N1C2CCCC1CC2] |
| 63 | 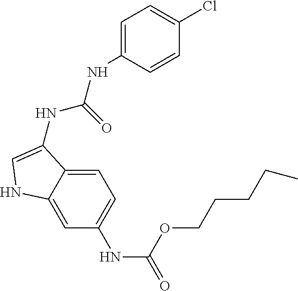 [SMILES: CCCCCOC(=O)Nc1ccc2c(NC(=O)Nc3ccc(Cl)cc3)c[nH]c2c1] |
| 64 | 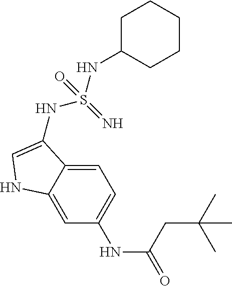 [SMILES: CC(C)(C)CC(=O)Nc1ccc2c(NS(=N)(=O)NC3CCCCC3)c[nH]c2c1] |
| 65 | 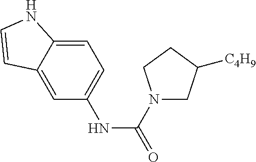 |
| 66 | 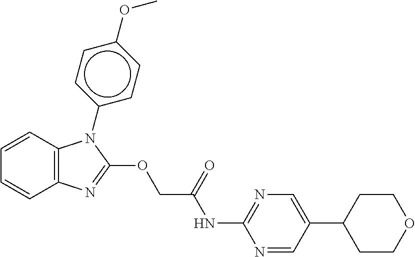 |
| 67 | 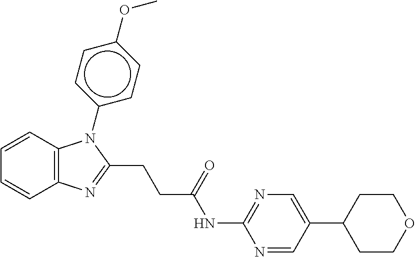 |
| 68 | 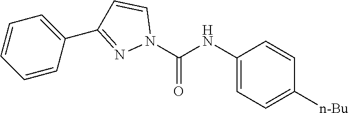 |
| 69 | 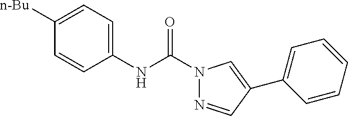 |
| 70 | 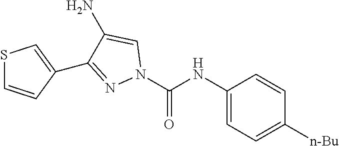 |
| 71 | 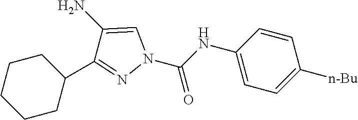 |
| 80 | 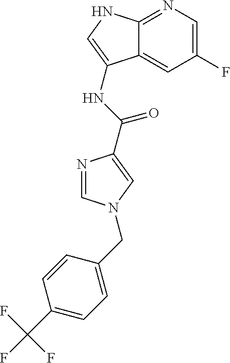 [SMILES: O=C(Nc1c[nH]c2ncc(F)cc12)c1cn(Cc2ccc(C(F)(F)F)cc2)cn1] |
| 81 | 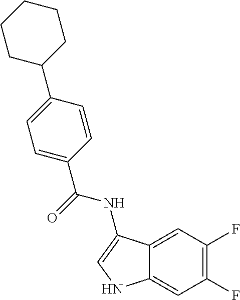 [SMILES: O=C(Nc1c[nH]c2cc(F)c(F)cc12)c1ccc(C2CCCCC2)cc1] |
| 82 | 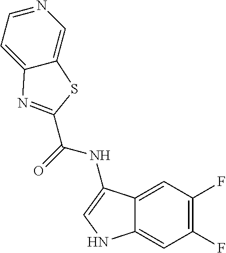 [SMILES: O=C(Nc1c[nH]c2cc(F)c(F)cc12)c1nc2ccncc2s1] |
| 83 | 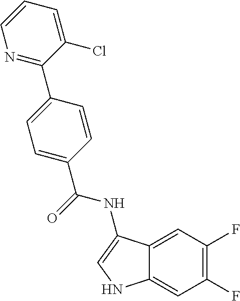 [SMILES: O=C(Nc1c[nH]c2cc(F)c(F)cc12)c1ccc(-c2ncccc2Cl)cc1] |
| 84 | 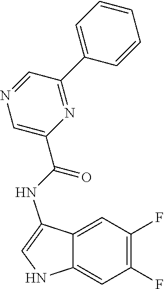 [SMILES: O=C(Nc1c[nH]c2cc(F)c(F)cc12)c1cncc(-c2ccccc2)n1] |
| 85 | 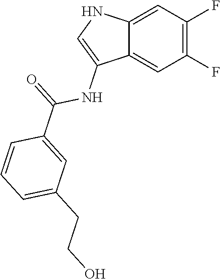 [SMILES: O=C(Nc1c[nH]c2cc(F)c(F)cc12)c1cccc(CCO)c1] |
| 86 | 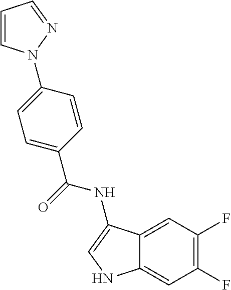 [SMILES: O=C(Nc1c[nH]c2cc(F)c(F)cc12)c1ccc(-n2cccn2)cc1] |
| 87 | 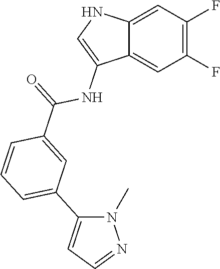 [SMILES: Cn1nccc1-c1cccc(C(=O)Nc2c[nH]c3cc(F)c(F)cc23)c1] |
| 88 | 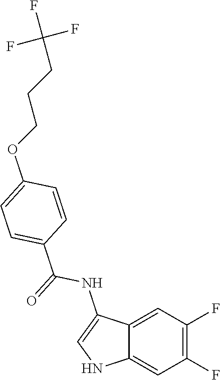 [SMILES: O=C(Nc1c[nH]c2cc(F)c(F)cc12)c1ccc(OCCCC(F)(F)F)cc1] |
| 89 | 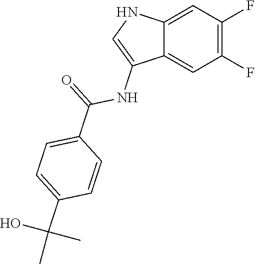 [SMILES: CC(C)(O)c1ccc(C(=O)Nc2c[nH]c3cc(F)c(F)cc23)cc1] |
| 90 | 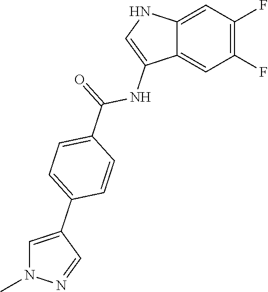 [SMILES: Cn1cc(-c2ccc(C(=O)Nc3c[nH]c4cc(F)c(F)cc34)cc2)cn1] |
| 91 | 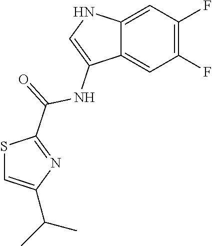 [SMILES: CC(C)c1csc(C(=O)Nc2c[nH]c3cc(F)c(F)cc23)n1] |
| 92 | 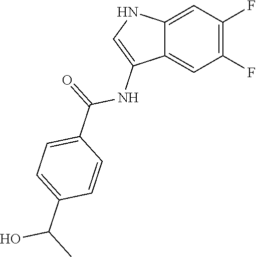 [SMILES: CC(O)c1ccc(C(=O)Nc2c[nH]c3cc(F)c(F)cc23)cc1] |
| 93 | 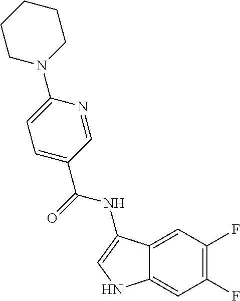 [SMILES: O=C(Nc1c[nH]c2cc(F)c(F)cc12)c1ccc(N2CCCCC2)nc1] |
| 94 | 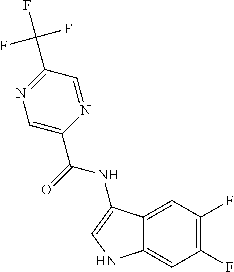 [SMILES: O=C(Nc1c[nH]c2cc(F)c(F)cc12)c1cnc(C(F)(F)F)cn1] |
| 95 | 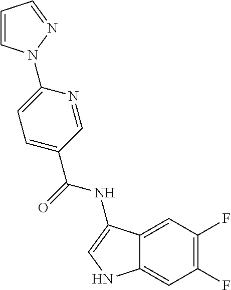 [SMILES: O=C(Nc1c[nH]c2cc(F)c(F)cc12)c1ccc(-n2cccn2)nc1] |
| 96 | 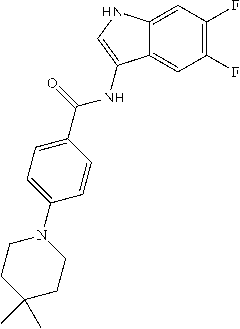 [SMILES: CC1(C)CCN(c2ccc(C(=O)Nc3c[nH]c4cc(F)c(F)cc34)cc2)CC1] |
| 97 | 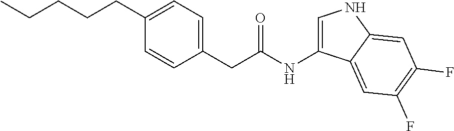 [SMILES: CCCCCc1ccc(CC(=O)Nc2c[nH]c3cc(F)c(F)cc23)cc1] |
| 98 | 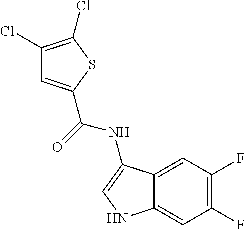 [SMILES: O=C(Nc1c[nH]c2cc(F)c(F)cc12)c1cc(Cl)c(Cl)s1] |
| 99 | 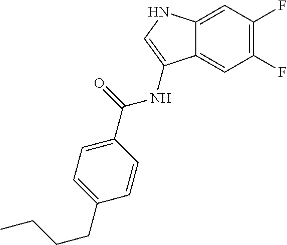 [SMILES: CCCCc1ccc(C(=O)Nc2c[nH]c3cc(F)c(F)cc23)cc1] |
| 100 | 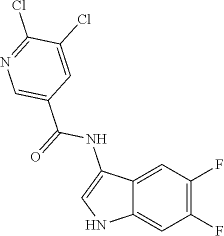 [SMILES: O=C(Nc1c[nH]c2cc(F)c(F)cc12)c1cnc(Cl)c(Cl)c1] |
| 101 | 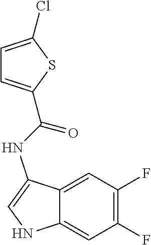 [SMILES: O=C(Nc1c[nH]c2cc(F)c(F)cc12)c1ccc(Cl)s1] |
| 102 | 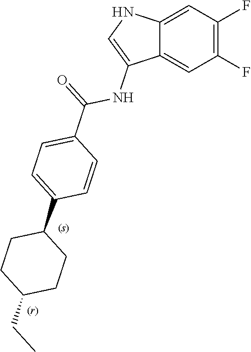 [SMILES: CC[C@H]1CC[C@H](c2ccc(C(=O)Nc3c[nH]c4cc(F)c(F)cc34)cc2)CC1] |
| 103 | 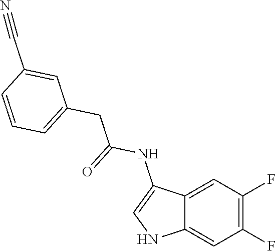 [SMILES: N#Cc1cccc(CC(=O)Nc2c[nH]c3cc(F)c(F)cc23)c1] |
| 104 | 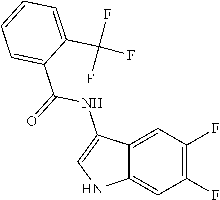 [SMILES: O=C(Nc1c[nH]c2cc(F)c(F)cc12)c1ccccc1C(F)(F)F] |
| 105 | 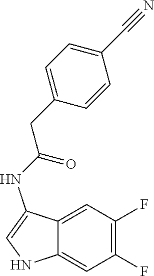 [SMILES: N#Cc1ccc(CC(=O)Nc2c[nH]c3cc(F)c(F)cc23)cc1] |
| 106 | 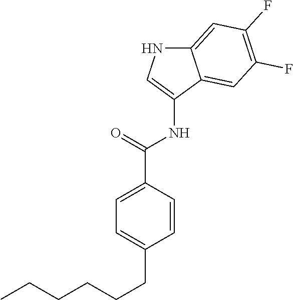 [SMILES: CCCCCCc1ccc(C(=O)Nc2c[nH]c3cc(F)c(F)cc23)cc1] |
| 107 | 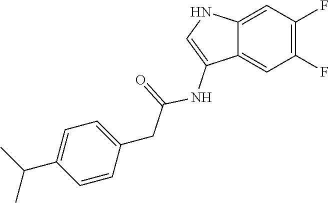 [SMILES: CC(C)c1ccc(CC(=O)Nc2c[nH]c3cc(F)c(F)cc23)cc1] |
| 108 | 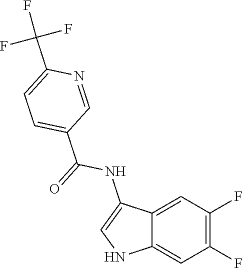 [SMILES: O=C(Nc1c[nH]c2cc(F)c(F)cc12)c1ccc(C(F)(F)F)nc1] |
| 109 | 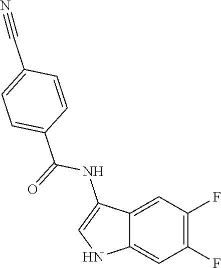 [SMILES: N#Cc1ccc(C(=O)Nc2c[nH]c3cc(F)c(F)cc23)cc1] |
| 110 | 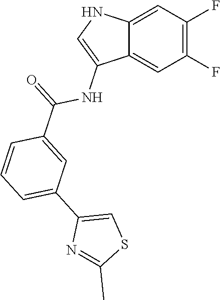 [SMILES: Cc1nc(-c2cccc(C(=O)Nc3c[nH]c4cc(F)c(F)cc34)c2)cs1] |
| 111 | 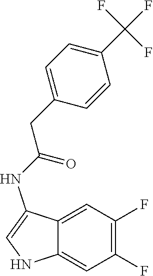 [SMILES: O=C(Cc1ccc(C(F)(F)F)cc1)Nc1c[nH]c2cc(F)c(F)cc12] |
| 112 | 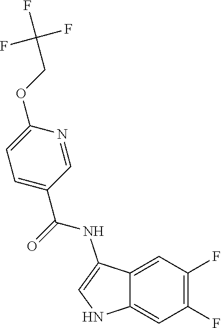 [SMILES: O=C(Nc1c[nH]c2cc(F)c(F)cc12)c1ccc(OCC(F)(F)F)nc1] |
| 113 | 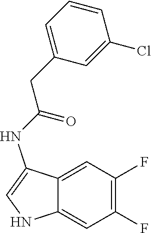 [SMILES: O=C(Cc1cccc(Cl)c1)Nc1c[nH]c2cc(F)c(F)cc12] |
| 114 | 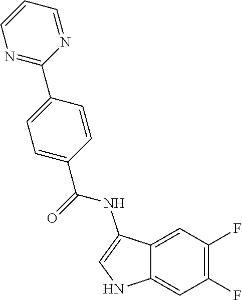 [SMILES: O=C(Nc1c[nH]c2cc(F)c(F)cc12)c1ccc(-c2ncccn2)cc1] |
| 115 | 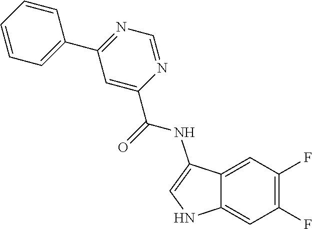 [SMILES: O=C(Nc1c[nH]c2cc(F)c(F)cc12)c1cc(-c2ccccc2)ncn1] |
| 116 | 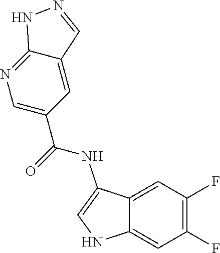 [SMILES: O=C(Nc1c[nH]c2cc(F)c(F)cc12)c1cnc2[nH]ncc2c1] |
| 117 | 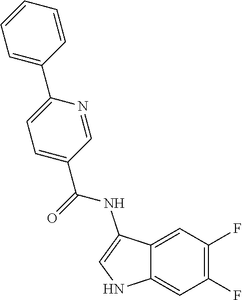 [SMILES: O=C(Nc1c[nH]c2cc(F)c(F)cc12)c1ccc(-c2ccccc2)nc1] |
| 118 | 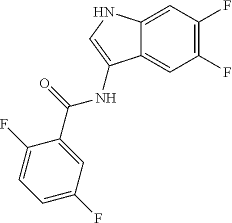 [SMILES: O=C(Nc1c[nH]c2cc(F)c(F)cc12)c1cc(F)ccc1F] |
| 119 | 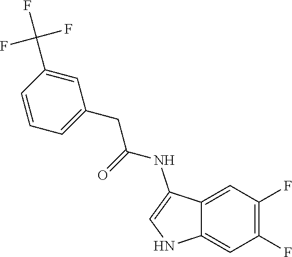 [SMILES: O=C(Cc1cccc(C(F)(F)F)c1)Nc1c[nH]c2cc(F)c(F)cc12] |
| 120 | 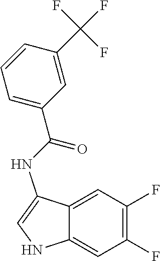 [SMILES: O=C(Nc1c[nH]c2cc(F)c(F)cc12)c1cccc(C(F)(F)F)c1] |
| 121 | 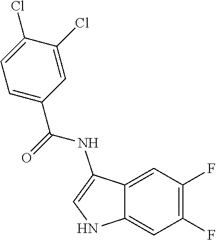 [SMILES: O=C(Nc1c[nH]c2cc(F)c(F)cc12)c1ccc(Cl)c(Cl)c1] |
| 122 | 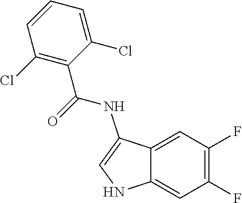 [SMILES: O=C(Nc1c[nH]c2cc(F)c(F)cc12)c1c(Cl)cccc1Cl] |
| 123 | 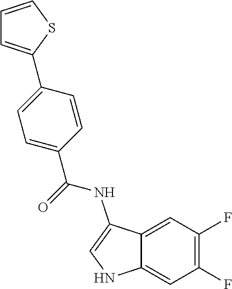 [SMILES: O=C(Nc1c[nH]c2cc(F)c(F)cc12)c1ccc(-c2cccs2)cc1] |
| 124 | 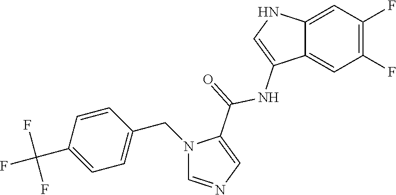 [SMILES: O=C(Nc1c[nH]c2cc(F)c(F)cc12)c1cncn1Cc1ccc(C(F)(F)F)cc1] |
| 125 | 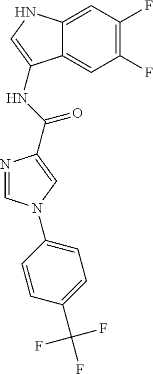 [SMILES: O=C(Nc1c[nH]c2cc(F)c(F)cc12)c1cn(-c2ccc(C(F)(F)F)cc2)cn1] |
| 126 | 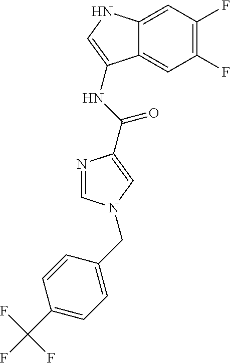 [SMILES: O=C(Nc1c[nH]c2cc(F)c(F)cc12)c1cn(Cc2ccc(C(F)(F)F)cc2)cn1] |
| 127 | 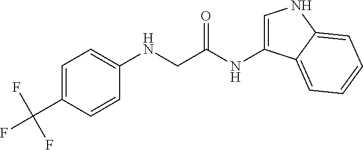 [SMILES: O=C(CNc1ccc(C(F)(F)F)cc1)Nc1c[nH]c2ccccc12] |
| 128 | 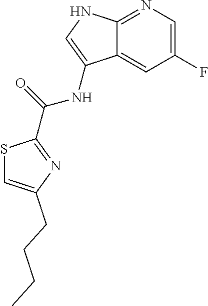 [SMILES: CCCCc1csc(C(=O)Nc2c[nH]c3ncc(F)cc23)n1] |
| 129 | 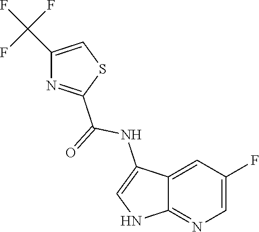 [SMILES: O=C(Nc1c[nH]c2ncc(F)cc12)c1nc(C(F)(F)F)cs1] |
| 130 | 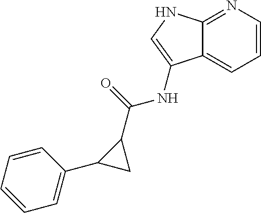 [SMILES: O=C(Nc1c[nH]c2ncccc12)C1CC1c1ccccc1] |
| 131 | 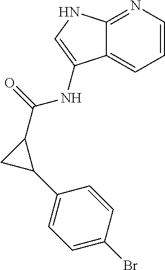 [SMILES: O=C(Nc1c[nH]c2ncccc12)C1CC1c1ccc(Br)cc1] |
| 132 | 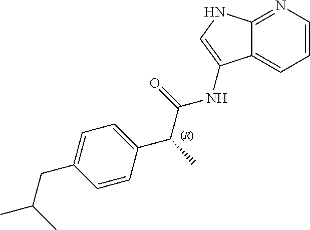 |
| 133 | 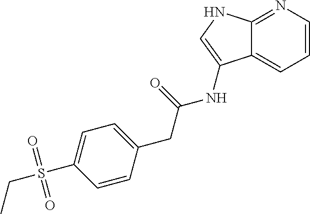 [SMILES: CCS(=O)(=O)c1ccc(CC(=O)Nc2c[nH]c3ncccc23)cc1] |
| 134 | 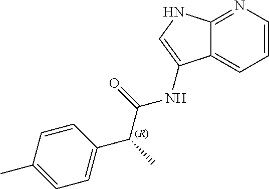 |
| 135 | 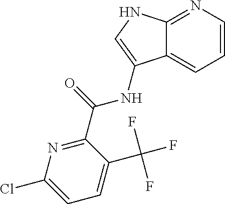 [SMILES: O=C(Nc1c[nH]c2ncccc12)c1nc(Cl)ccc1C(F)(F)F] |
| 136 | 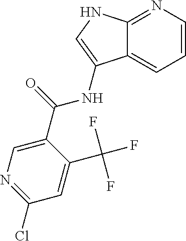 [SMILES: O=C(Nc1c[nH]c2ncccc12)c1cnc(Cl)cc1C(F)(F)F] |
| 137 | 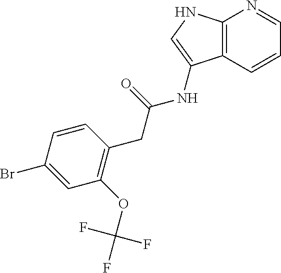 [SMILES: O=C(Cc1ccc(Br)cc1OC(F)(F)F)Nc1c[nH]c2ncccc12] |
| 138 | 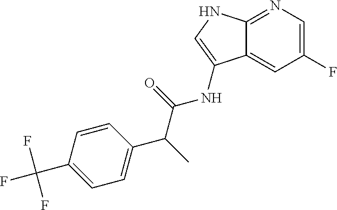 [SMILES: CC(C(=O)Nc1c[nH]c2ncc(F)cc12)c1ccc(C(F)(F)F)cc1] |
| 139 | 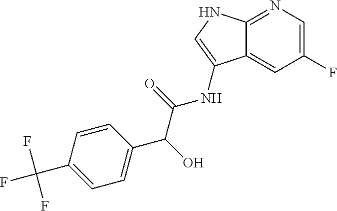 [SMILES: O=C(Nc1c[nH]c2ncc(F)cc12)C(O)c1ccc(C(F)(F)F)cc1] |
| 140 | 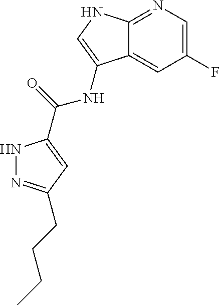 [SMILES: CCCCc1cc(C(=O)Nc2c[nH]c3ncc(F)cc23)[nH]n1] |
| 141 | 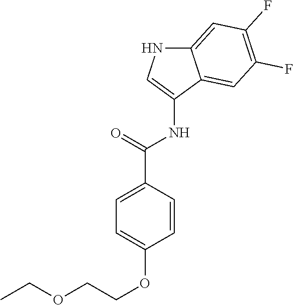 [SMILES: CCOCCOc1ccc(C(=O)Nc2c[nH]c3cc(F)c(F)cc23)cc1] |
| 142 | 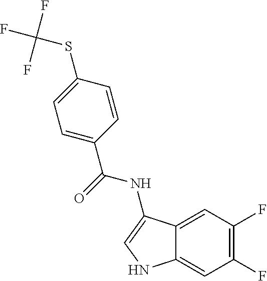 [SMILES: O=C(Nc1c[nH]c2cc(F)c(F)cc12)c1ccc(SC(F)(F)F)cc1] |
| 143 | 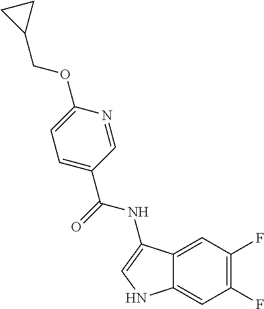 [SMILES: O=C(Nc1c[nH]c2cc(F)c(F)cc12)c1ccc(OCC2CC2)nc1] |
| 144 | 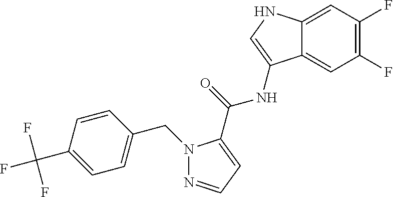 [SMILES: O=C(Nc1c[nH]c2cc(F)c(F)cc12)c1ccnn1Cc1ccc(C(F)(F)F)cc1] |
| 145 | 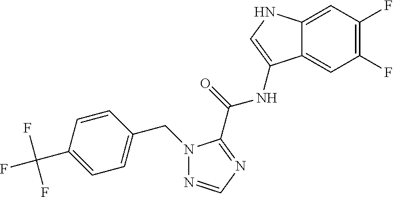 [SMILES: O=C(Nc1c[nH]c2cc(F)c(F)cc12)c1ncnn1Cc1ccc(C(F)(F)F)cc1] |
| 146 | 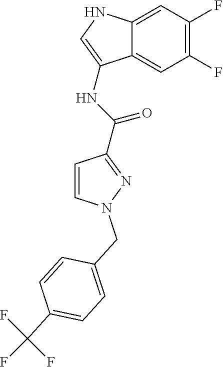 [SMILES: O=C(Nc1c[nH]c2cc(F)c(F)cc12)c1ccn(Cc2ccc(C(F)(F)F)cc2)n1] |
| 147 | 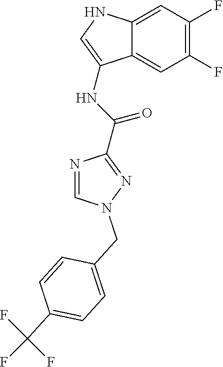 [SMILES: O=C(Nc1c[nH]c2cc(F)c(F)cc12)c1ncn(Cc2ccc(C(F)(F)F)cc2)n1] |
| 148 | 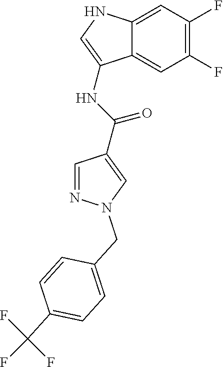 [SMILES: O=C(Nc1c[nH]c2cc(F)c(F)cc12)c1cnn(Cc2ccc(C(F)(F)F)cc2)c1] |
| 149 | 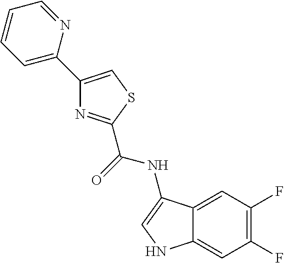 [SMILES: O=C(Nc1c[nH]c2cc(F)c(F)cc12)c1nc(-c2ccccn2)cs1] |
| 150 | 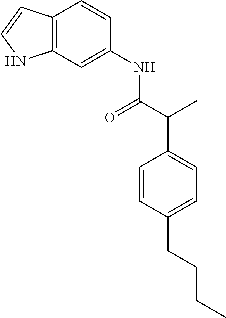 [SMILES: CCCCc1ccc(C(C)C(=O)Nc2ccc3cc[nH]c3c2)cc1] |
| 151 | 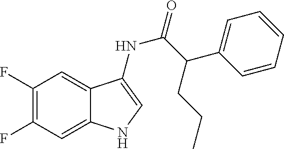 [SMILES: CCCC(C(=O)Nc1c[nH]c2cc(F)c(F)cc12)c1ccccc1] |
| 152 | 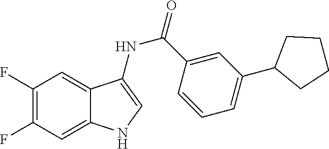 [SMILES: O=C(Nc1c[nH]c2cc(F)c(F)cc12)c1cccc(C2CCCC2)c1] |
| 153 | 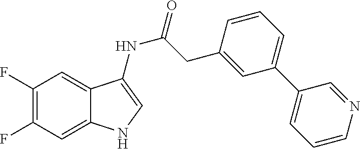 [SMILES: O=C(Cc1cccc(-c2cccnc2)c1)Nc1c[nH]c2cc(F)c(F)cc12] |
| 154 | 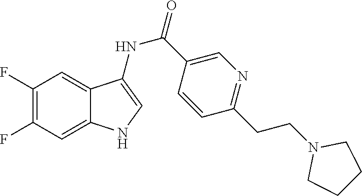 [SMILES: O=C(Nc1c[nH]c2cc(F)c(F)cc12)c1ccc(CCN2CCCC2)nc1] |
| 155 | 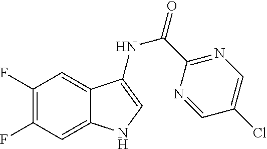 [SMILES: O=C(Nc1c[nH]c2cc(F)c(F)cc12)c1ncc(Cl)cn1] |
| 156 | 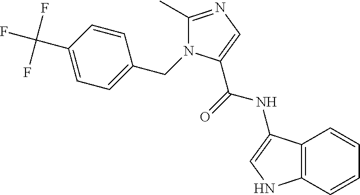 [SMILES: Cc1ncc(C(=O)Nc2c[nH]c3ccccc23)n1Cc1ccc(C(F)(F)F)cc1] |
| 157 | 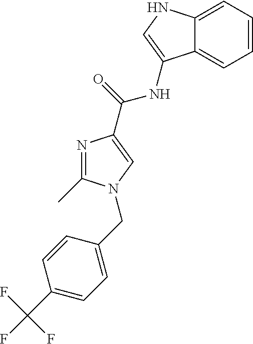 [SMILES: Cc1nc(C(=O)Nc2c[nH]c3ccccc23)cn1Cc1ccc(C(F)(F)F)cc1] |
| 158 | 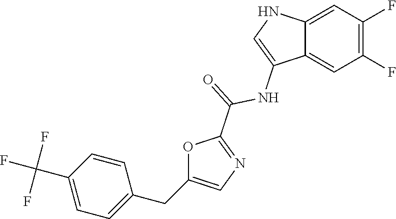 [SMILES: O=C(Nc1c[nH]c2cc(F)c(F)cc12)c1ncc(Cc2ccc(C(F)(F)F)cc2)o1] |
| 159 | 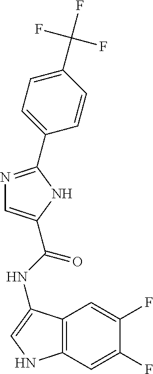 [SMILES: O=C(Nc1c[nH]c2cc(F)c(F)cc12)c1cnc(-c2ccc(C(F)(F)F)cc2)[nH]1] |
| 160 | 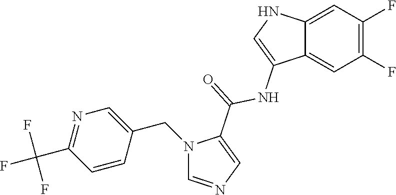 [SMILES: O=C(Nc1c[nH]c2cc(F)c(F)cc12)c1cncn1Cc1ccc(C(F)(F)F)nc1] |
| 161 | 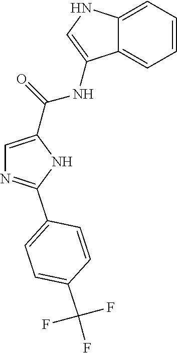 [SMILES: O=C(Nc1c[nH]c2ccccc12)c1cnc(-c2ccc(C(F)(F)F)cc2)[nH]1] |
| 162 | 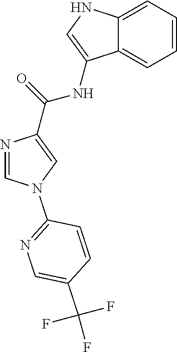 [SMILES: O=C(Nc1c[nH]c2ccccc12)c1cn(-c2ccc(C(F)(F)F)cn2)cn1] |
| 163 | 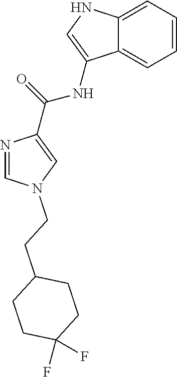 [SMILES: O=C(Nc1c[nH]c2ccccc12)c1cn(CCC2CCC(F)(F)CC2)cn1] |
| 164 | 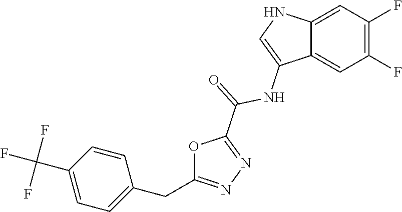 [SMILES: O=C(Nc1c[nH]c2cc(F)c(F)cc12)c1nnc(Cc2ccc(C(F)(F)F)cc2)o1] |
| 165 | 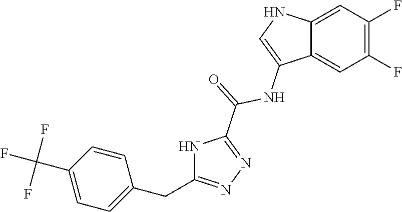 [SMILES: O=C(Nc1c[nH]c2cc(F)c(F)cc12)c1nnc(Cc2ccc(C(F)(F)F)cc2)[nH]1] |
| 166 | 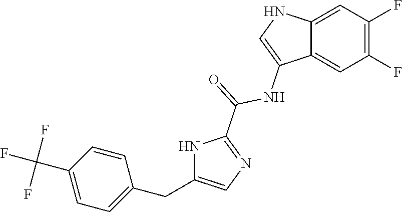 [SMILES: O=C(Nc1c[nH]c2cc(F)c(F)cc12)c1ncc(Cc2ccc(C(F)(F)F)cc2)[nH]1] |
| 167 | 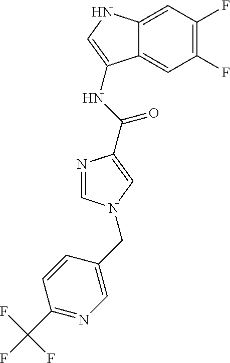 [SMILES: O=C(Nc1c[nH]c2cc(F)c(F)cc12)c1cn(Cc2ccc(C(F)(F)F)nc2)cn1] |
| 168 | 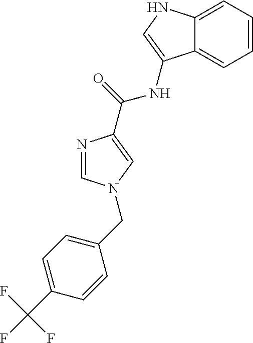 [SMILES: O=C(Nc1c[nH]c2ccccc12)c1cn(Cc2ccc(C(F)(F)F)cc2)cn1] |

Compounds of Formula (III), Formula (IV), Table 3, and methods of making and using the same are further described in PCT/US2020/013786, filed on Jan. 16, 2020; U.S. Provisional 62/793,795, filed on Jan. 17, 2019; U.S. Provisional 62/861,865, filed on Jun. 14, 2019; U.S. Provisional 62/869,914, filed on Jul. 2, 2019; and U.S. Provisional 62/955,891, filed on Dec. 31, 2019, each of which is incorporated herein by reference in its entirety.

In one aspect, the STING antagonist is a compound of Formula (V):

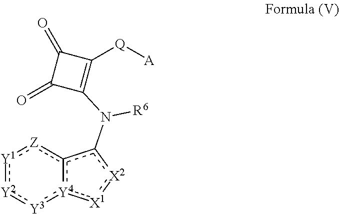

or a pharmaceutically acceptable salt thereof or a tautomer thereof,

or a pharmaceutically acceptable salt thereof or a tautomer thereof, wherein:

Z is selected from the group consisting of a bond, CR¹, C(R³)₂, N, and NR²;
each of Y¹, Y², and Y³ is independently selected from the group consisting of O, S, CR¹, C(R³)₂, N, and NR²;

### Y⁴ is C or N;

X¹ is selected from the group consisting of O, S, N, NR², and CR¹;
X² is selected from the group consisting of O, S, N, NR⁴, and CR⁵;
each [symbol] is independently a single bond or a double bond, provided that the five-membered ring comprising Y⁴, X¹, and X² is heteroaryl;
Q-A is defined according to (A) or (B) below:

### A

Q is selected from the group consisting of: NH; N(C₁₋₆ alkyl) wherein the C₁₋₆ alkyl is optionally substituted with 1-2 independently selected R^{a}; O; S; and C₁₋₃ alkylene which is optionally substituted with 1-2 independently selected R^{a} and

### A is:

(i) —(Y^{A1})ₙ—Y^{A2}, wherein:
n is 0 or 1;
Y^{A1} is C₁₋₆ alkylene, which is optionally substituted with from 1-6 substituents each independently selected from the group consisting of R^{a}; C₆₋₁₀ aryl optionally substituted with 1-4 independently selected C₁₋₄ alkyl; and heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected C₁₋₄ alkyl; and
Y^{A2} is:
   (a) C₃₋₂₀ cycloalkyl, which is optionally substituted with from 1-4 R^{b},
   (b) C₆₋₂₀ aryl, which is optionally substituted with from 1-4 R^{c};
   (c) heteroaryl including from 5-20 ring atoms, wherein from 1-3 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; or
   (d) heterocyclyl including from 3-16 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},

### OR

(ii) —Z¹—Z²—Z³, wherein:
Z¹ is C₁₋₃ alkylene, which is optionally substituted with from 1-4 R^{a};
Z² is —N(H)—, —N(R^{d})—, —O—, or —S—; and
Z³ is C₂₋₇ alkyl, which is optionally substituted with from 1-4 R^{a};

### OR

(iii) C₁₋₁₀ alkyl, which is optionally substituted with from 1-6 independently selected R^{a},

### OR

### B

Q and A, taken together, form:

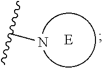 [SMILES: *N]

and
E is heterocyclyl including from 3-16 ring atoms, wherein aside from the nitrogen atom present, from 0-3 additional ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},
each occurrence of R¹ is independently selected from the group consisting of
H;
halo;
cyano;
C₁₋₆ alkyl optionally substituted with 1-2 R^{a};
C₂₋₆ alkenyl;
C₂₋₆ alkynyl;
C₁₋₄ haloalkyl;
C₁₋₄ alkoxy;
C₁₋₄ haloalkoxy;
-L³-L⁴-Rⁱ;
—S(O)₁₋₂(C₁₋₄ alkyl),
—S(O)(═NH)(C₁₋₄ alkyl),
SF₅,
—NR^{e}R^{f},
—OH,
oxo,
—S(O)₁₋₂(NR′R″),
—C₁₋₄ thioalkoxy,
—NO₂,
—C(═O)(C₁₋₄ alkyl),
—C(═O)O(C₁₋₄ alkyl),
—C(═O)OH, and
—C(═O)N(R′)(R″);
   or a pair of R¹ on adjacent atoms, taken together with the atoms connecting them, form a ring including from 3-10 ring atoms, wherein from 0-2 ring atoms are heteroatoms each independently selected from the group consisting of N, N(H), N(R^{d}), O, and S(O)₀₋₂; and wherein the ring is optionally substituted with from 1-4 substituents each independently selected from C₁₋₆ alkyl, halo, C₁₋₆ haloalkyl, —OH, NR^{e}R^{f}, C₁₋₆ alkoxy, and C₁₋₆ haloalkoxy,
   each occurrence of R² is independently selected from the group consisting of:
   (i) C₁₋₆ alkyl, which is optionally substituted with from 1-2 independently selected R^{a};
   (ii) C₃₋₆ cycloalkyl;
   (iii) heterocyclyl including from 3-10 ring atoms, wherein from 1-3 ring atoms are heteroatoms, each independently selected from the group consisting of N, N(H), N(R^{d}), O, and S(O)₀₋₂;
   (iv) C₆₋₁₀ aryl;
   (v) heteroaryl including from 5-10 ring atoms, wherein from 1-3 ring atoms are heteroatoms, each independently selected from the group consisting of N, N(H), N(R^{d}), O, and S(O)₀₋₂;
   (vi) —C(O)(C₁₋₄ alkyl);
   (vii) —C(O)O(C₁₋₄ alkyl);
   (viii) —CON(R′R″);

### (ix) —S(O)₁₋₂(NR′R″);

(x) —S(O)₁₋₂(C₁₋₄ alkyl);

### (xi) —OH;

(xii) C₁₋₄ alkoxy; and
(xiii) H;
or a pair of R¹ and R² on adjacent atoms, taken together with the atoms connecting them, form a ring including from 3-10 ring atoms, wherein from 0-2 ring atoms (in addition to the nitrogen atom to which the R² is attached) are heteroatoms each independently selected from the group consisting of N, N(H), N(R^{d}), O, and S(O)₀₋₂; and wherein the ring is optionally substituted with from 1-4 substituents each independently selected from C₁₋₆ alkyl, halo, C₁₋₆ haloalkyl, —OH, NR^{e}R^{f}, C₁₋₆ alkoxy, and C₁₋₆ haloalkoxy,
each occurrence of R³ is independently selected from H; C₁₋₆ alkyl optionally substituted with from 1-6 independently selected R^{a}; C₁₋₄ haloalkyl; —OH; —F; —Cl; —Br; —NR^{e}R^{f}; C₁₋₄ alkoxy; C₁₋₄ haloalkoxy; —C(═O)(C₁₋₄ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and C₃₋₆ cycloalkyl optionally substituted with from 1-4 independently selected C₁₋₄ alkyl; or two R³ on the same carbon combine to form an oxo; or

a pair of R³, taken together with the atom(s) connecting them, form a ring including from 3-10 ring atoms, wherein from 0-2 ring atoms are heteroatoms each independently selected from the group consisting of N, N(H), N(R^{d}), O, and S(O)₀₋₂; and wherein the ring is optionally substituted with from 1-4 substituents each independently selected from C₁₋₆ alkyl, halo, C₁₋₆ haloalkyl, —OH, NR^{e}R^{f}, C₁₋₆ alkoxy, and C₁₋₆ haloalkoxy; or

a pair of R¹ and R³ on adjacent atoms, taken together with the atoms connecting them, form a ring including from 3-10 ring atoms, wherein from 0-2 ring atoms are heteroatoms each independently selected from the group consisting of N, N(H), N(R^{d}), O, and S(O)₀₋₂; and wherein the ring is optionally substituted with from 1-4 substituents each independently selected from C₁₋₆ alkyl, halo, C₁₋₆ haloalkyl, —OH, NR^{e}R^{f}, C₁₋₆ alkoxy, and C₁₋₆ haloalkoxy; or

or a pair of R² and R³ on adjacent atoms, taken together with the atoms connecting them, form a ring including from 3-10 ring atoms, wherein from 0-2 ring atoms (in addition to the nitrogen atom to which the R² is attached) are heteroatoms each independently selected from the group consisting of N, N(H), N(R^{d}), O, and S(O)₀₋₂; and wherein the ring is optionally substituted with from 1-4 substituents each independently selected from C₁₋₆ alkyl, halo, C₁₋₆ haloalkyl, —OH, NR^{e}R^{f}, C₁₋₆ alkoxy, and C₁₋₆ haloalkoxy;
R⁴ is selected from H and C₁₋₆ alkyl;
R⁵ is selected from H and halo;
R⁶ is selected from H; C₁₋₆ alkyl; —OH; C₁₋₄ alkoxy; C(═O)H; C(═O)(C₁₋₄ alkyl); CN; C₆₋₁₀ aryl optionally substituted with from 1-4 independently selected C₁₋₄ alkyl; and heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 independently selected C₁₋₄ alkyl;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;

each occurrence of R^{b} is independently selected from the group consisting of: C₁₋₁₀ alkyl optionally substituted with from 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of:

(a) halo; (b) cyano; (c) C₁₋₁₀ alkyl which is optionally substituted with from 1-6 independently selected R^{a}; (d) C₂₋₆ alkenyl; (e) C₂₋₆ alkynyl; (g) C₁₋₄ alkoxy; (h) C₁₋₄ haloalkoxy; (i) —S(O)₁₋₂(C₁₋₄ alkyl); (j) —NR^{e}R^{f}; (k) —OH; (l) —S(O)₁₋₂(NR′R″); (m) —C₁₋₄ thioalkoxy; (n) —NO₂; (o) —C(═O)(C₁₋₁₀ alkyl); (p) —C(═O)O(C₁₋₄ alkyl); (q) —C(═O)OH; (r) —C(═O)N(R′)(R″); and (s) -L¹-L²-R^{h};

R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene;
-L² is —O—, —N(H)—, —S(O)₀₋₂—, or a bond;
R^{h} is selected from:
C₃₋₈ cycloalkyl optionally substituted with from 1-4 substituents independently selected from the group consisting of halo, C₁₋₄ alkyl, and C₁₋₄ haloalkyl (in certain embodiments, it is provided that when R^{h} is C₃₋₆ cycloalkyl optionally substituted with from 1-4 substituents independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo, C₁₋₄ alkyl, and C₁₋₄ haloalkyl;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo, C₁. 4 alkyl, and C₁₋₄ haloalkyl; and
C₆₋₁₀ aryl, which is optionally substituted with from 1-4 substituents independently selected from the group consisting of halo, C₁₋₄ alkyl, and C₁₋₄ haloalkyl;
   -L³ is a bond or C₁₋₃ alkylene;
   -L⁴ is —O—, —N(H)—, —S(O)₀₋₂—, or a bond;
   R¹ is selected from:
C₃₋₈ cycloalkyl optionally substituted with from 1-4 substituents independently selected from the group consisting of halo, C₁₋₄ alkyl, and C₁₋₄ haloalkyl (in certain embodiments, it is provided that when Rⁱ is C₃₋₆ cycloalkyl optionally substituted with from 1-4 substituents independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo, C₁₋₄ alkyl, and C₁₋₄ haloalkyl;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo, C₁. 4 alkyl, and C₁₋₄ haloalkyl; and
C₆₋₁₀ aryl, which is optionally substituted with from 1-4 substituents independently selected from the group consisting of halo, C₁₋₄ alkyl, or C₁₋₄ haloalkyl; and

each occurrence of R′ and R″ is independently selected from the group consisting of: H, C₁₋₄ alkyl, and C₆₋₁₀ aryl optionally substituted with from 1-2 substituents selected from halo, C₁₋₄ alkyl, and C₁₋₄ haloalkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(C₁₋₆ alkyl), O, and S.

In another aspect, the STING antagonist is a compound selected from the group consisting of compounds in Table 4 and pharmaceutically acceptable salts thereof.

TABLE 4

| Compound | Structure |
|---|---|
| 101 | 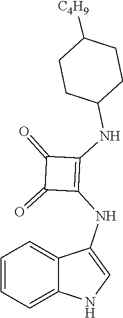 |
| 102 | 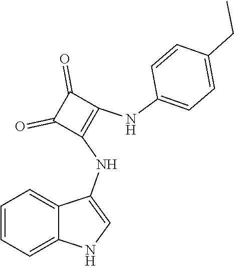 [SMILES: CCc1ccc(Nc2c(Nc3c[nH]c4ccccc34)c(=O)c2=O)cc1] |
| 103 | 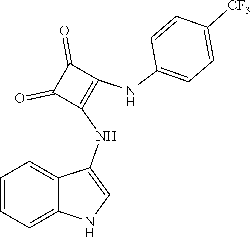 [SMILES: O=c1c(Nc2ccc(C(F)(F)F)cc2)c(Nc2c[nH]c3ccccc23)c1=O] |
| 104 | 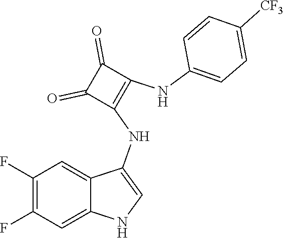 [SMILES: O=c1c(Nc2ccc(C(F)(F)F)cc2)c(Nc2c[nH]c3cc(F)c(F)cc23)c1=O] |
| 105 | 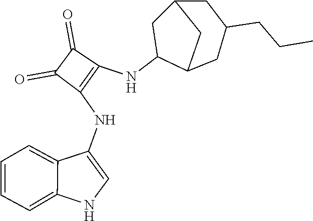 [SMILES: CCCC1CC2CC(C1)C(Nc1c(Nc3c[nH]c4ccccc34)c(=O)c1=O)C2] |
| 106 | 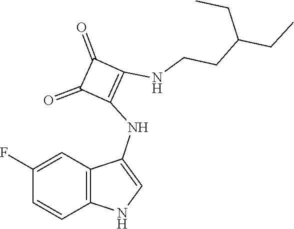 [SMILES: CCC(CC)CCNc1c(Nc2c[nH]c3ccc(F)cc23)c(=O)c1=O] |
| 107 | 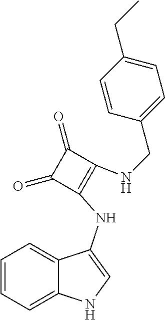 [SMILES: CCc1ccc(CNc2c(Nc3c[nH]c4ccccc34)c(=O)c2=O)cc1] |
| 108 | 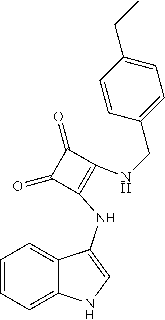 [SMILES: CCc1ccc(CNc2c(Nc3c[nH]c4ccccc34)c(=O)c2=O)cc1] |
| 109 | 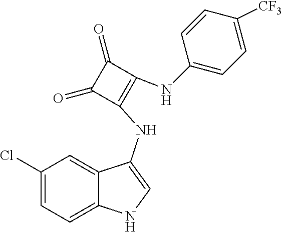 [SMILES: O=c1c(Nc2ccc(C(F)(F)F)cc2)c(Nc2c[nH]c3ccc(Cl)cc23)c1=O] |
| 110 | 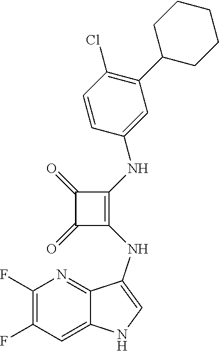 [SMILES: O=c1c(Nc2ccc(Cl)c(C3CCCCC3)c2)c(Nc2c[nH]c3cc(F)c(F)nc23)c1=O] |
| 111 | 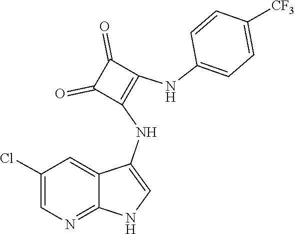 [SMILES: O=c1c(Nc2ccc(C(F)(F)F)cc2)c(Nc2c[nH]c3ncc(Cl)cc23)c1=O] |
| 112 | 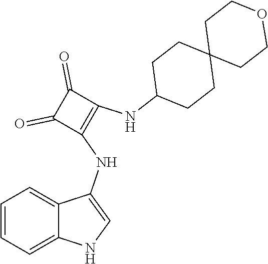 [SMILES: O=c1c(Nc2c[nH]c3ccccc23)c(NC2CCC3(CCOCC3)CC2)c1=O] |
| 113 | 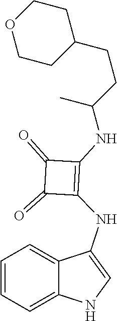 [SMILES: CC(CCC1CCOCC1)Nc1c(Nc2c[nH]c3ccccc23)c(=O)c1=O] |
| 114 | 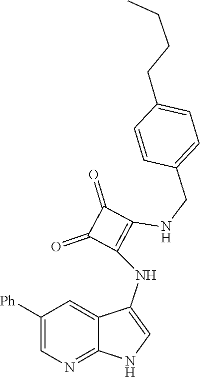 [SMILES: CCCCc1ccc(CNc2c(Nc3c[nH]c4ncc(-c5ccccc5)cc34)c(=O)c2=O)cc1] |
| 115 | 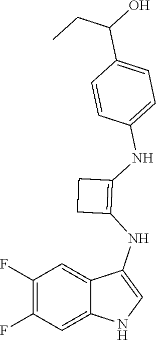 [SMILES: CCC(O)c1ccc(NC2=C(Nc3c[nH]c4cc(F)c(F)cc34)CC2)cc1] |
| 116 | 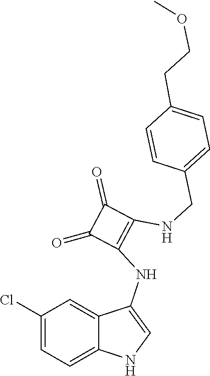 [SMILES: COCCc1ccc(CNc2c(Nc3c[nH]c4ccc(Cl)cc34)c(=O)c2=O)cc1] |
| 117 | 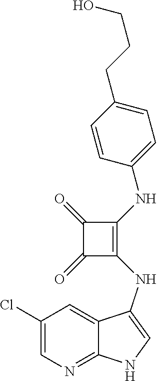 [SMILES: O=c1c(Nc2ccc(CCCO)cc2)c(Nc2c[nH]c3ncc(Cl)cc23)c1=O] |
| 118 | 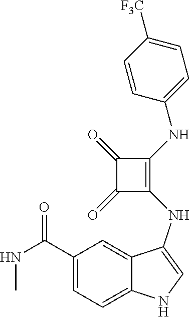 [SMILES: CNC(=O)c1ccc2[nH]cc(Nc3c(Nc4ccc(C(F)(F)F)cc4)c(=O)c3=O)c2c1] |
| 119 | 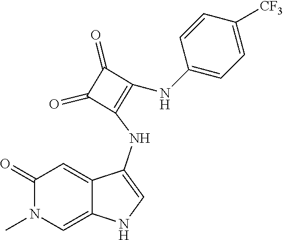 [SMILES: Cn1cc2[nH]cc(Nc3c(Nc4ccc(C(F)(F)F)cc4)c(=O)c3=O)c2cc1=O] |
| 120 | 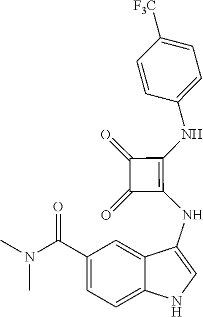 [SMILES: CN(C)C(=O)c1ccc2[nH]cc(Nc3c(Nc4ccc(C(F)(F)F)cc4)c(=O)c3=O)c2c1] |
| 121 | 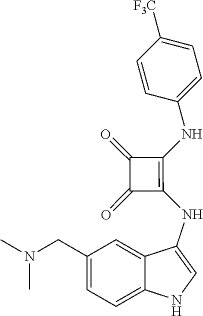 [SMILES: CN(C)Cc1ccc2[nH]cc(Nc3c(Nc4ccc(C(F)(F)F)cc4)c(=O)c3=O)c2c1] |
| 122 | 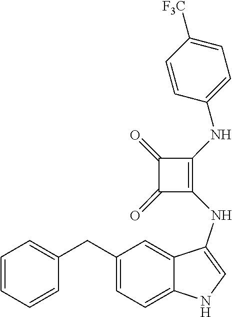 [SMILES: O=c1c(Nc2ccc(C(F)(F)F)cc2)c(Nc2c[nH]c3ccc(Cc4ccccc4)cc23)c1=O] |
| 123 | 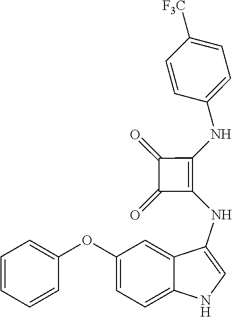 [SMILES: O=c1c(Nc2ccc(C(F)(F)F)cc2)c(Nc2c[nH]c3ccc(Oc4ccccc4)cc23)c1=O] |
| 124 | 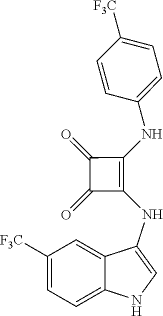 [SMILES: O=c1c(Nc2ccc(C(F)(F)F)cc2)c(Nc2c[nH]c3ccc(C(F)(F)F)cc23)c1=O] |
| 125 | 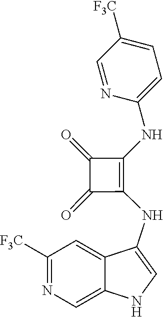 [SMILES: O=c1c(Nc2ccc(C(F)(F)F)cn2)c(Nc2c[nH]c3cnc(C(F)(F)F)cc23)c1=O] |
| 126 | 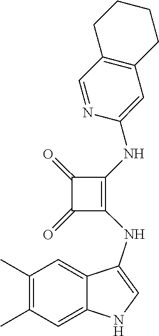 [SMILES: Cc1cc2[nH]cc(Nc3c(Nc4cc5c(cn4)CCCC5)c(=O)c3=O)c2cc1C] |
| 127 | 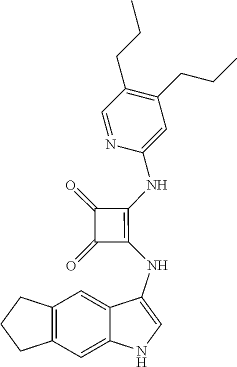 [SMILES: CCCc1cnc(Nc2c(Nc3c[nH]c4cc5c(cc34)CCC5)c(=O)c2=O)cc1CCC] |
| 128 | 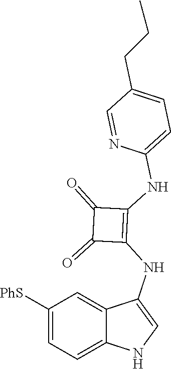 |
| 129 | 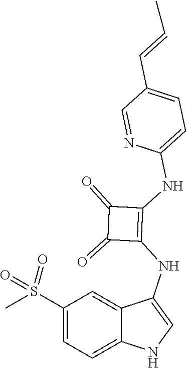 [SMILES: C/C=C/c1ccc(Nc2c(Nc3c[nH]c4ccc(S(C)(=O)=O)cc34)c(=O)c2=O)nc1] |
| 130 | 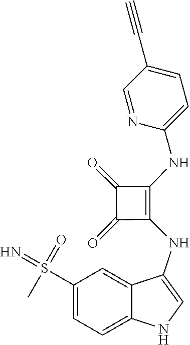 [SMILES: C#Cc1ccc(Nc2c(Nc3c[nH]c4ccc(S(C)(=N)=O)cc34)c(=O)c2=O)nc1] |
| 131 | 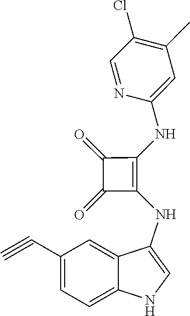 [SMILES: C#Cc1ccc2[nH]cc(Nc3c(Nc4cc(C)c(Cl)cn4)c(=O)c3=O)c2c1] |
| 132 | 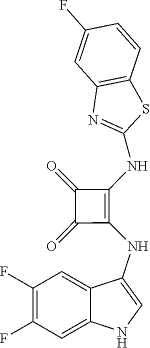 [SMILES: O=c1c(Nc2nc3cc(F)ccc3s2)c(Nc2c[nH]c3cc(F)c(F)cc23)c1=O] |
| 133 | 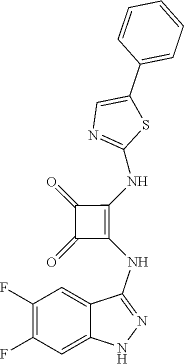 [SMILES: O=c1c(Nc2ncc(-c3ccccc3)s2)c(Nc2n[nH]c3cc(F)c(F)cc23)c1=O] |
| 134 | 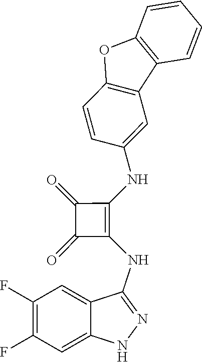 [SMILES: O=c1c(Nc2ccc3oc4ccccc4c3c2)c(Nc2n[nH]c3cc(F)c(F)cc23)c1=O] |
| 135 | 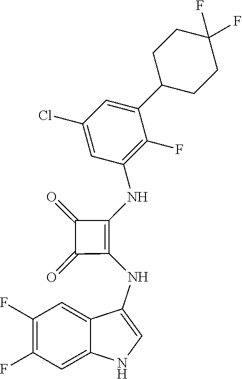 [SMILES: O=c1c(Nc2cc(Cl)cc(C3CCC(F)(F)CC3)c2F)c(Nc2c[nH]c3cc(F)c(F)cc23)c1=O] |
| 136 | 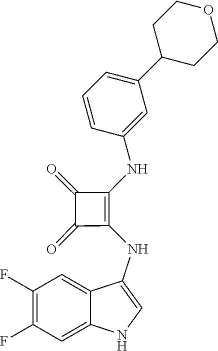 [SMILES: O=c1c(Nc2cccc(C3CCOCC3)c2)c(Nc2c[nH]c3cc(F)c(F)cc23)c1=O] |
| 137 | 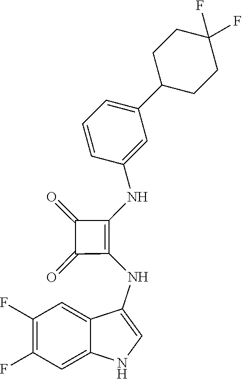 [SMILES: O=c1c(Nc2cccc(C3CCC(F)(F)CC3)c2)c(Nc2c[nH]c3cc(F)c(F)cc23)c1=O] |
| 138 | 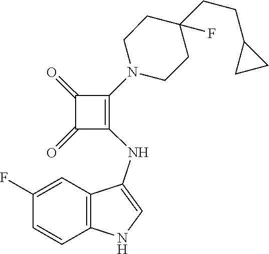 [SMILES: O=c1c(Nc2c[nH]c3ccc(F)cc23)c(N2CCC(F)(CCC3CC3)CC2)c1=O] |
| 139 | 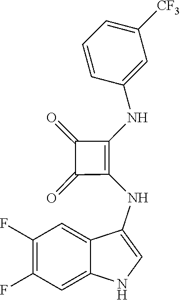 [SMILES: O=c1c(Nc2cccc(C(F)(F)F)c2)c(Nc2c[nH]c3cc(F)c(F)cc23)c1=O] |
| 140 | 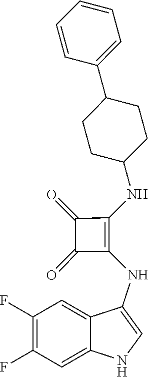 [SMILES: O=c1c(Nc2c[nH]c3cc(F)c(F)cc23)c(NC2CCC(c3ccccc3)CC2)c1=O] |
| 141 | 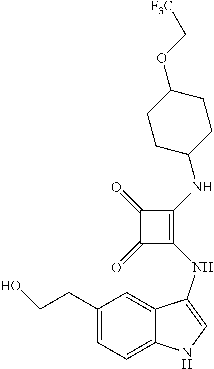 [SMILES: O=c1c(Nc2c[nH]c3ccc(CCO)cc23)c(NC2CCC(OCC(F)(F)F)CC2)c1=O] |
| 142 | 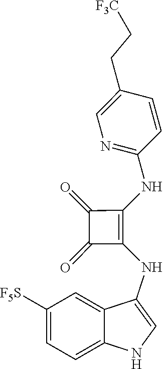 |
| 143 | 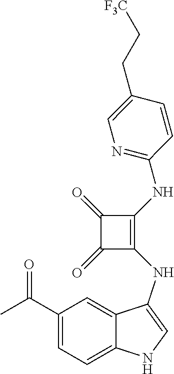 [SMILES: CC(=O)c1ccc2[nH]cc(Nc3c(Nc4ccc(CCC(F)(F)F)cn4)c(=O)c3=O)c2c1] |
| 144 | 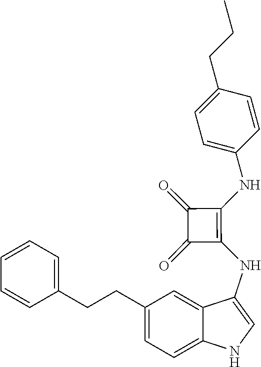 [SMILES: CCCc1ccc(Nc2c(Nc3c[nH]c4ccc(CCc5ccccc5)cc34)c(=O)c2=O)cc1] |
| 145 | 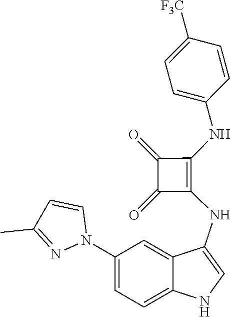 [SMILES: Cc1ccn(-c2ccc3[nH]cc(Nc4c(Nc5ccc(C(F)(F)F)cc5)c(=O)c4=O)c3c2)n1] |
| 146 | 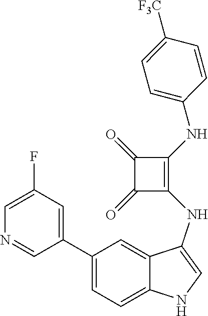 [SMILES: O=c1c(Nc2ccc(C(F)(F)F)cc2)c(Nc2c[nH]c3ccc(-c4cncc(F)c4)cc23)c1=O] |
| 147 | 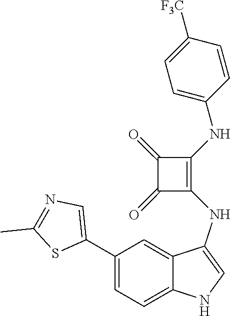 [SMILES: Cc1ncc(-c2ccc3[nH]cc(Nc4c(Nc5ccc(C(F)(F)F)cc5)c(=O)c4=O)c3c2)s1] |
| 148 | 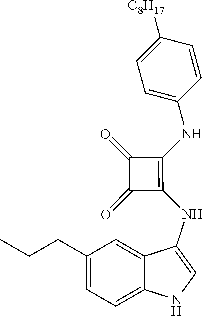 |
| 149 | 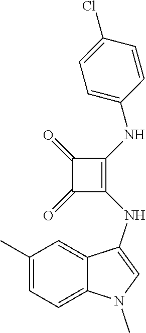 [SMILES: Cc1ccc2c(c1)c(Nc1c(Nc3ccc(Cl)cc3)c(=O)c1=O)cn2C] |
| 150 | 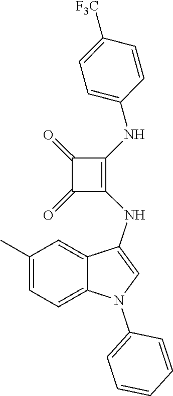 [SMILES: Cc1ccc2c(c1)c(Nc1c(Nc3ccc(C(F)(F)F)cc3)c(=O)c1=O)cn2-c1ccccc1] |
| 151 | 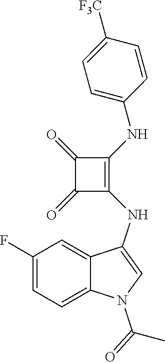 [SMILES: CC(=O)n1cc(Nc2c(Nc3ccc(C(F)(F)F)cc3)c(=O)c2=O)c2cc(F)ccc21] |
| 152 | 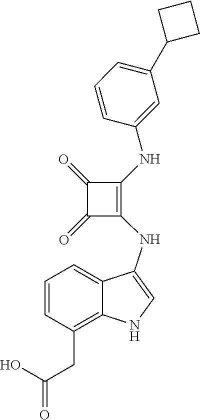 [SMILES: O=C(O)Cc1cccc2c(Nc3c(Nc4cccc(C5CCC5)c4)c(=O)c3=O)c[nH]c12] |
| 153 | 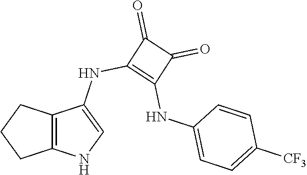 [SMILES: O=c1c(Nc2ccc(C(F)(F)F)cc2)c(Nc2c[nH]c3c2CCC3)c1=O] |
| 154 | 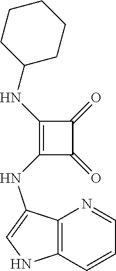 [SMILES: O=c1c(Nc2c[nH]c3cccnc23)c(NC2CCCCC2)c1=O] |
| 155 | 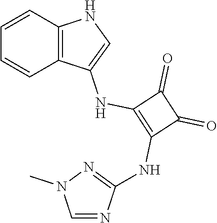 [SMILES: Cn1cnc(Nc2c(Nc3c[nH]c4ccccc34)c(=O)c2=O)n1] |
| 156 | 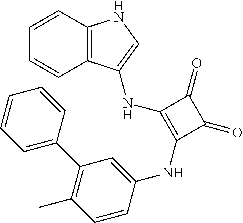 [SMILES: Cc1ccc(Nc2c(Nc3c[nH]c4ccccc34)c(=O)c2=O)cc1-c1ccccc1] |
| 157 | 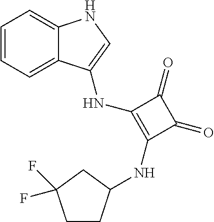 [SMILES: O=c1c(Nc2c[nH]c3ccccc23)c(NC2CCC(F)(F)C2)c1=O] |
| 157a | 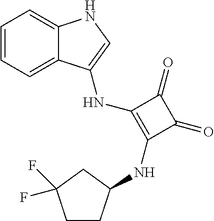 [SMILES: O=c1c(Nc2c[nH]c3ccccc23)c(N[C@H]2CCC(F)(F)C2)c1=O] |
| 157b | 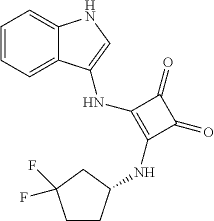 [SMILES: O=c1c(Nc2c[nH]c3ccccc23)c(N[C@@H]2CCC(F)(F)C2)c1=O] |
| 158 | 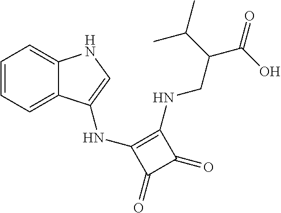 [SMILES: CC(C)C(CNc1c(Nc2c[nH]c3ccccc23)c(=O)c1=O)C(=O)O] |
| 158a | 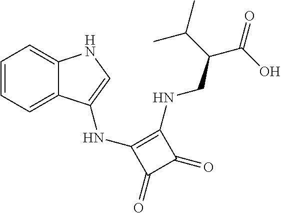 [SMILES: CC(C)[C@H](CNc1c(Nc2c[nH]c3ccccc23)c(=O)c1=O)C(=O)O] |
| 158b | 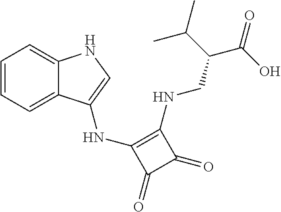 [SMILES: CC(C)[C@@H](CNc1c(Nc2c[nH]c3ccccc23)c(=O)c1=O)C(=O)O] |
| 159 | 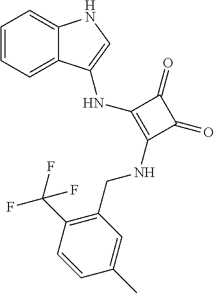 [SMILES: Cc1ccc(C(F)(F)F)c(CNc2c(Nc3c[nH]c4ccccc34)c(=O)c2=O)c1] |
| 160 | 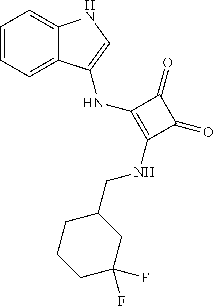 [SMILES: O=c1c(NCC2CCCC(F)(F)C2)c(Nc2c[nH]c3ccccc23)c1=O] |
| 160a | 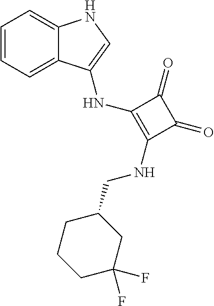 [SMILES: O=c1c(NC[C@H]2CCCC(F)(F)C2)c(Nc2c[nH]c3ccccc23)c1=O] |
| 160b | 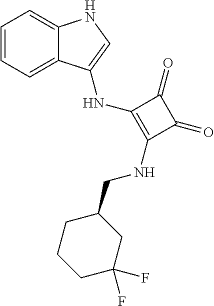 [SMILES: O=c1c(NC[C@@H]2CCCC(F)(F)C2)c(Nc2c[nH]c3ccccc23)c1=O] |
| 161 | 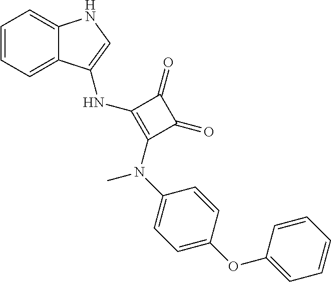 [SMILES: CN(c1ccc(Oc2ccccc2)cc1)c1c(Nc2c[nH]c3ccccc23)c(=O)c1=O] |
| 162 | 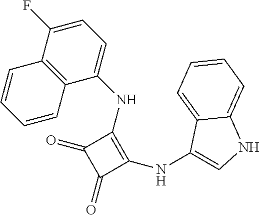 [SMILES: O=c1c(Nc2c[nH]c3ccccc23)c(Nc2ccc(F)c3ccccc23)c1=O] |
| 163 | 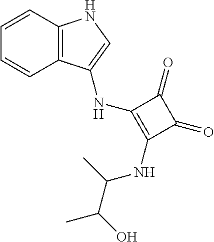 [SMILES: CC(O)C(C)Nc1c(Nc2c[nH]c3ccccc23)c(=O)c1=O] |
| 164 | 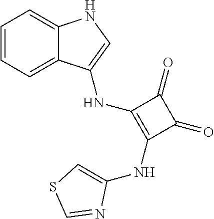 [SMILES: O=c1c(Nc2cscn2)c(Nc2c[nH]c3ccccc23)c1=O] |
| 165 | 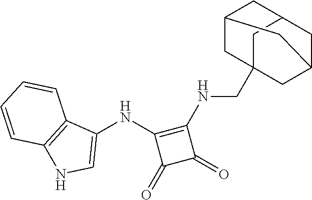 [SMILES: O=c1c(NCC23CC4CC(CC(C4)C2)C3)c(Nc2c[nH]c3ccccc23)c1=O] |
| 166 | 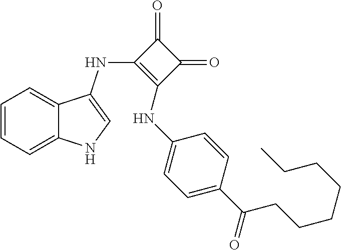 [SMILES: CCCCCCCC(=O)c1ccc(Nc2c(Nc3c[nH]c4ccccc34)c(=O)c2=O)cc1] |
| 167 | 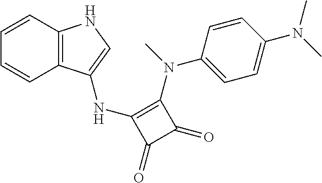 [SMILES: CN(C)c1ccc(N(C)c2c(Nc3c[nH]c4ccccc34)c(=O)c2=O)cc1] |
| 168 | 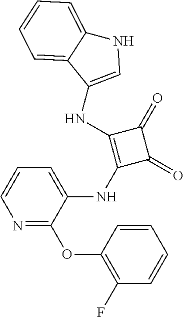 [SMILES: O=c1c(Nc2cccnc2Oc2ccccc2F)c(Nc2c[nH]c3ccccc23)c1=O] |
| 169 | 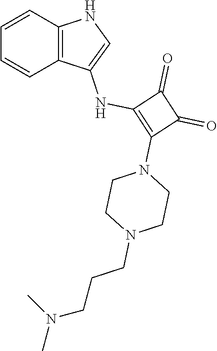 [SMILES: CN(C)CCCN1CCN(c2c(Nc3c[nH]c4ccccc34)c(=O)c2=O)CC1] |
| 170 | 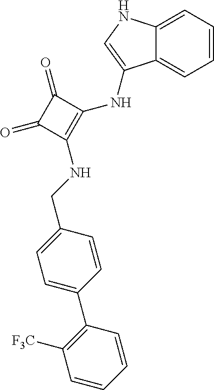 [SMILES: O=c1c(NCc2ccc(-c3ccccc3C(F)(F)F)cc2)c(Nc2c[nH]c3ccccc23)c1=O] |
| 171 | 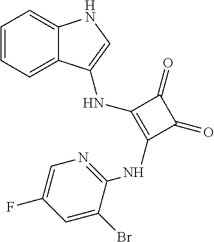 [SMILES: O=c1c(Nc2ncc(F)cc2Br)c(Nc2c[nH]c3ccccc23)c1=O] |
| 172 | 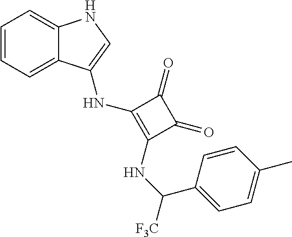 [SMILES: Cc1ccc(C(Nc2c(Nc3c[nH]c4ccccc34)c(=O)c2=O)C(F)(F)F)cc1] |
| 172a | 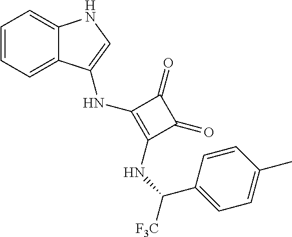 [SMILES: Cc1ccc([C@@H](Nc2c(Nc3c[nH]c4ccccc34)c(=O)c2=O)C(F)(F)F)cc1] |
| 172b | 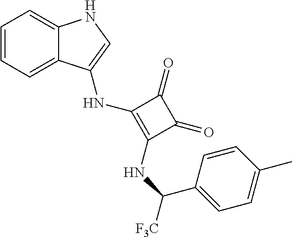 [SMILES: Cc1ccc([C@H](Nc2c(Nc3c[nH]c4ccccc34)c(=O)c2=O)C(F)(F)F)cc1] |
| 173 | 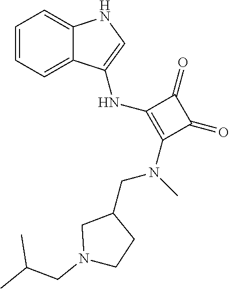 [SMILES: CC(C)CN1CCC(CN(C)c2c(Nc3c[nH]c4ccccc34)c(=O)c2=O)C1] |
| 173a | 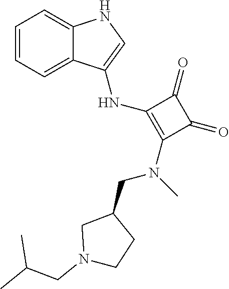 [SMILES: CC(C)CN1CC[C@H](CN(C)c2c(Nc3c[nH]c4ccccc34)c(=O)c2=O)C1] |
| 173b | 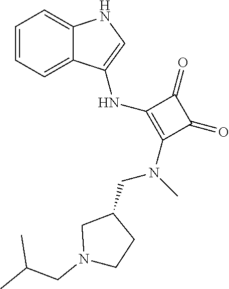 [SMILES: CC(C)CN1CC[C@@H](CN(C)c2c(Nc3c[nH]c4ccccc34)c(=O)c2=O)C1] |
| 174 | 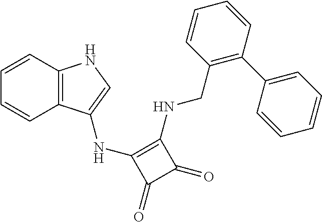 [SMILES: O=c1c(NCc2ccccc2-c2ccccc2)c(Nc2c[nH]c3ccccc23)c1=O] |
| 175 | 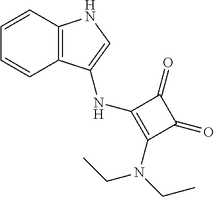 [SMILES: CCN(CC)c1c(Nc2c[nH]c3ccccc23)c(=O)c1=O] |
| 176 | 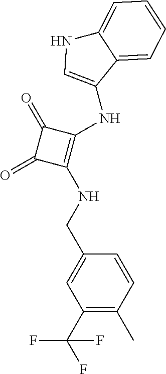 [SMILES: Cc1ccc(CNc2c(Nc3c[nH]c4ccccc34)c(=O)c2=O)cc1C(F)(F)F] |
| 177 | 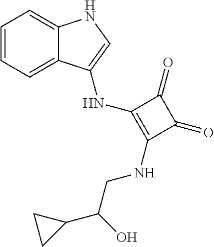 [SMILES: O=c1c(NCC(O)C2CC2)c(Nc2c[nH]c3ccccc23)c1=O] |
| 177a | 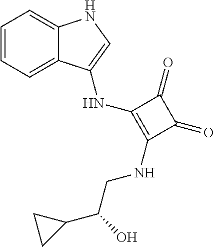 [SMILES: O=c1c(NC[C@H](O)C2CC2)c(Nc2c[nH]c3ccccc23)c1=O] |
| 177b | 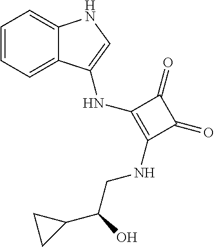 [SMILES: O=c1c(NC[C@@H](O)C2CC2)c(Nc2c[nH]c3ccccc23)c1=O] |
| 178 | 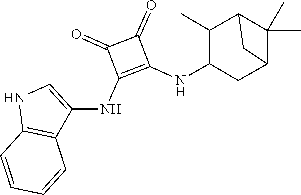 [SMILES: CC1C(Nc2c(Nc3c[nH]c4ccccc34)c(=O)c2=O)CC2CC1C2(C)C] |
| 178a | 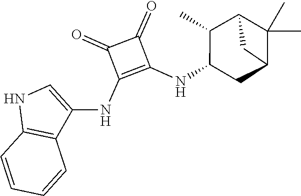 [SMILES: C[C@H]1[C@@H](Nc2c(Nc3c[nH]c4ccccc34)c(=O)c2=O)C[C@H]2C[C@@H]1C2(C)C] |
| 178b | 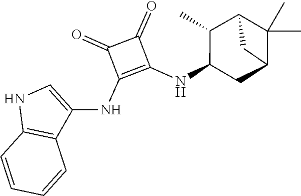 [SMILES: C[C@H]1[C@H](Nc2c(Nc3c[nH]c4ccccc34)c(=O)c2=O)C[C@H]2C[C@@H]1C2(C)C] |
| 179 | 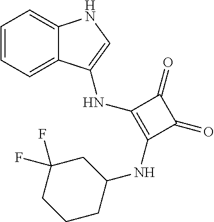 [SMILES: O=c1c(Nc2c[nH]c3ccccc23)c(NC2CCCC(F)(F)C2)c1=O] |
| 179a | 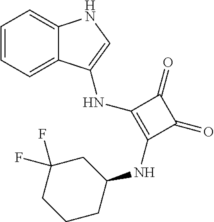 [SMILES: O=c1c(Nc2c[nH]c3ccccc23)c(N[C@H]2CCCC(F)(F)C2)c1=O] |
| 179b | 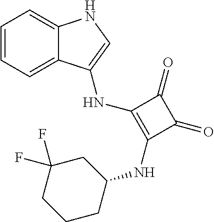 [SMILES: O=c1c(Nc2c[nH]c3ccccc23)c(N[C@@H]2CCCC(F)(F)C2)c1=O] |
| 180 | 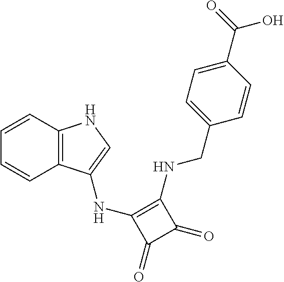 [SMILES: O=C(O)c1ccc(CNc2c(Nc3c[nH]c4ccccc34)c(=O)c2=O)cc1] |
| 181 | 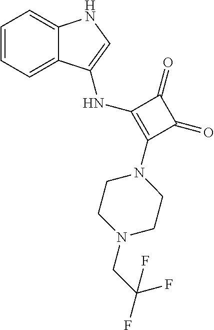 [SMILES: O=c1c(Nc2c[nH]c3ccccc23)c(N2CCN(CC(F)(F)F)CC2)c1=O] |
| 182 | 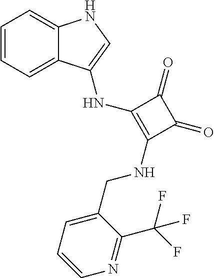 [SMILES: O=c1c(NCc2cccnc2C(F)(F)F)c(Nc2c[nH]c3ccccc23)c1=O] |
| 183 | 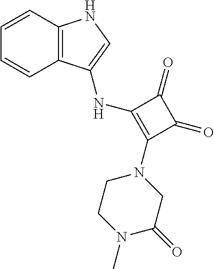 [SMILES: CN1CCN(c2c(Nc3c[nH]c4ccccc34)c(=O)c2=O)CC1=O] |
| 184 | 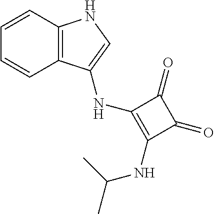 [SMILES: CC(C)Nc1c(Nc2c[nH]c3ccccc23)c(=O)c1=O] |
| 185 | 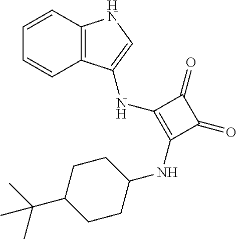 [SMILES: CC(C)(C)C1CCC(Nc2c(Nc3c[nH]c4ccccc34)c(=O)c2=O)CC1] |
| 186 | 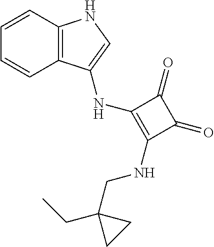 [SMILES: CCC1(CNc2c(Nc3c[nH]c4ccccc34)c(=O)c2=O)CC1] |
| 187 | 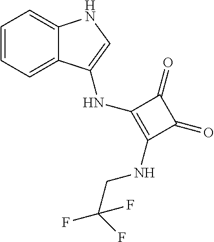 [SMILES: O=c1c(NCC(F)(F)F)c(Nc2c[nH]c3ccccc23)c1=O] |
| 188 | 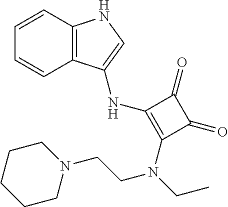 [SMILES: CCN(CCN1CCCCC1)c1c(Nc2c[nH]c3ccccc23)c(=O)c1=O] |
| 189 | 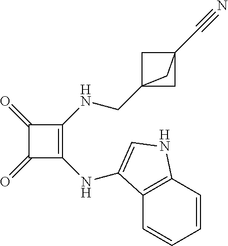 [SMILES: N#CC12CC(CNc3c(Nc4c[nH]c5ccccc45)c(=O)c3=O)(C1)C2] |
| 190 | 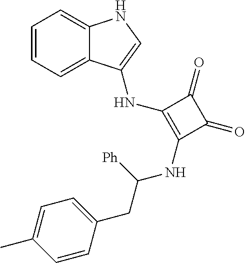 [SMILES: Cc1ccc(CC(Nc2c(Nc3c[nH]c4ccccc34)c(=O)c2=O)c2ccccc2)cc1] |
| 190a | 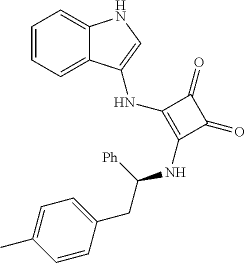 [SMILES: Cc1ccc(C[C@H](Nc2c(Nc3c[nH]c4ccccc34)c(=O)c2=O)c2ccccc2)cc1] |
| 190b | 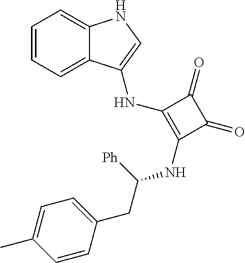 [SMILES: Cc1ccc(C[C@@H](Nc2c(Nc3c[nH]c4ccccc34)c(=O)c2=O)c2ccccc2)cc1] |
| 191 | 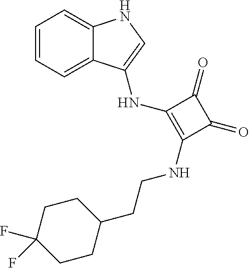 [SMILES: O=c1c(NCCC2CCC(F)(F)CC2)c(Nc2c[nH]c3ccccc23)c1=O] |
| 192 | 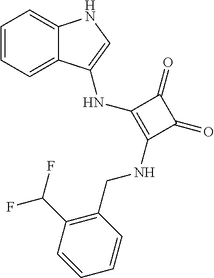 [SMILES: O=c1c(NCc2ccccc2C(F)F)c(Nc2c[nH]c3ccccc23)c1=O] |
| 193 | 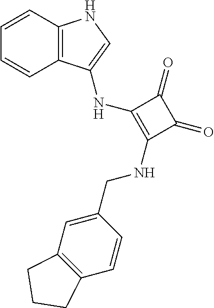 [SMILES: O=c1c(NCc2ccc3c(c2)CCC3)c(Nc2c[nH]c3ccccc23)c1=O] |
| 194 | 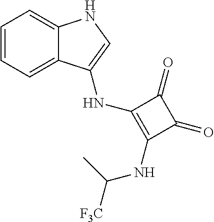 [SMILES: CC(Nc1c(Nc2c[nH]c3ccccc23)c(=O)c1=O)C(F)(F)F] |
| 194a | 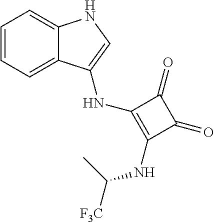 [SMILES: C[C@H](Nc1c(Nc2c[nH]c3ccccc23)c(=O)c1=O)C(F)(F)F] |
| 194b | 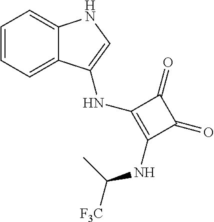 [SMILES: C[C@@H](Nc1c(Nc2c[nH]c3ccccc23)c(=O)c1=O)C(F)(F)F] |
| 195 | 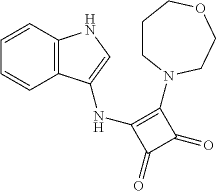 [SMILES: O=c1c(Nc2c[nH]c3ccccc23)c(N2CCCOCC2)c1=O] |
| 196 | 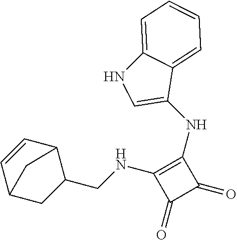 [SMILES: O=c1c(NCC2CC3C=CC2C3)c(Nc2c[nH]c3ccccc23)c1=O] |
| 197 | 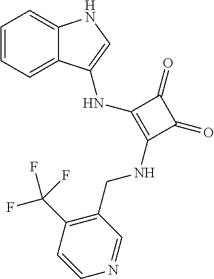 [SMILES: O=c1c(NCc2cnccc2C(F)(F)F)c(Nc2c[nH]c3ccccc23)c1=O] |
| 198 | 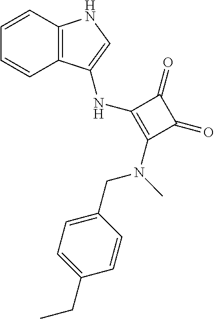 [SMILES: CCc1ccc(CN(C)c2c(Nc3c[nH]c4ccccc34)c(=O)c2=O)cc1] |
| 199 | 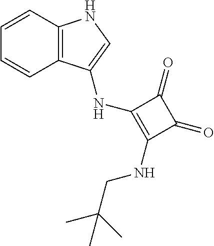 [SMILES: CC(C)(C)CNc1c(Nc2c[nH]c3ccccc23)c(=O)c1=O] |
| 200 | 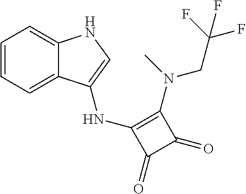 [SMILES: CN(CC(F)(F)F)c1c(Nc2c[nH]c3ccccc23)c(=O)c1=O] |
| 201 | 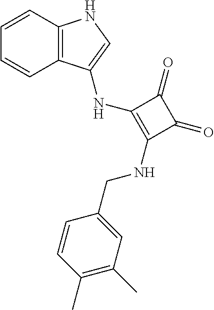 [SMILES: Cc1ccc(CNc2c(Nc3c[nH]c4ccccc34)c(=O)c2=O)cc1C] |
| 202 | 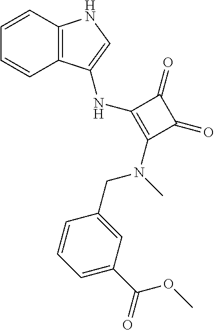 [SMILES: COC(=O)c1cccc(CN(C)c2c(Nc3c[nH]c4ccccc34)c(=O)c2=O)c1] |
| 203 | 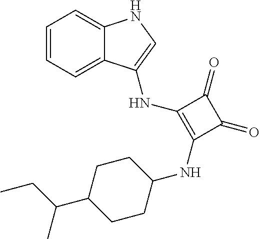 [SMILES: CCC(C)C1CCC(Nc2c(Nc3c[nH]c4ccccc34)c(=O)c2=O)CC1] |
| 203a | 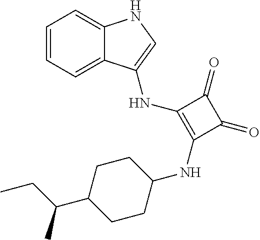 [SMILES: CC[C@H](C)C1CCC(Nc2c(Nc3c[nH]c4ccccc34)c(=O)c2=O)CC1] |
| 203b | 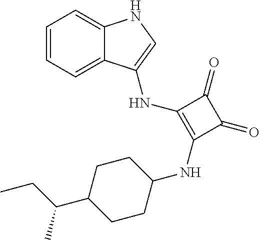 [SMILES: CC[C@@H](C)C1CCC(Nc2c(Nc3c[nH]c4ccccc34)c(=O)c2=O)CC1] |
| 204 | 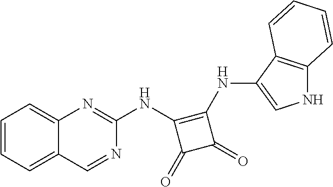 [SMILES: O=c1c(Nc2ncc3ccccc3n2)c(Nc2c[nH]c3ccccc23)c1=O] |
| 205 | 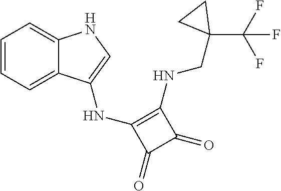 [SMILES: O=c1c(NCC2(C(F)(F)F)CC2)c(Nc2c[nH]c3ccccc23)c1=O] |
| 206 | 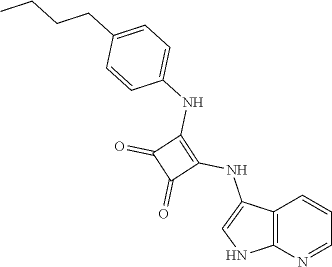 [SMILES: CCCCc1ccc(Nc2c(Nc3c[nH]c4ncccc34)c(=O)c2=O)cc1] |
| 207 | 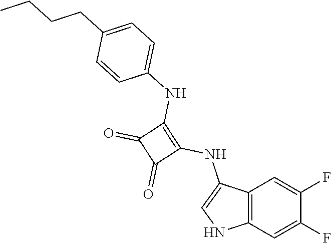 [SMILES: CCCCc1ccc(Nc2c(Nc3c[nH]c4cc(F)c(F)cc34)c(=O)c2=O)cc1] |
| 208 | 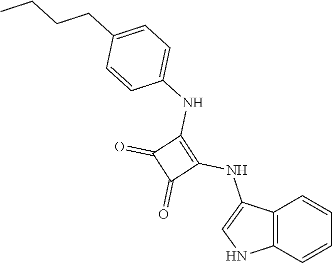 [SMILES: CCCCc1ccc(Nc2c(Nc3c[nH]c4ccccc34)c(=O)c2=O)cc1] |

Compounds of Formula (V) and Table 4, and methods of making and using the same are further described in PCT/US2020/033127, filed on May 15, 2020; U.S. Provisional 62/849,811, filed on May 17, 2019 and U.S. Provisional 62/861,880, filed on Jun. 14, 2019, each of which is incorporated herein by reference in its entirety.

In another aspect, the STING antagonist is a compound of Formula (VI):

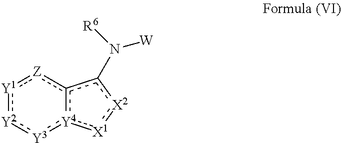

or a pharmaceutically acceptable salt thereof or a tautomer thereof,

wherein:

Z is selected from the group consisting of a bond, CR¹, C(R³)₂, N, and NR²;

each of Y¹, Y², and Y³ is independently selected from the group consisting of O, S, CR¹, C(R³)₂, N, and NR²;

Y⁴ is C or N;

X¹ is selected from the group consisting of O, S, N, NR², and CR¹;

X² is selected from the group consisting of O, S, N, NR⁴, and CR⁵;

each [symbol] is independently a single bond or a double bond, provided that the five-membered ring comprising Y⁴, X¹, and X² is heteroaryl;

W is defined according to (A) or (B) below:

### A

W is Q¹-Q²-A, wherein

Q¹ is selected from the group consisting of:
(a) phenyl optionally substituted with from 1-2 independently selected Rai; and
(b) heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected Rai;

Q² is selected from the group consisting of: a bond, —NH—, —N(C₁₋₃ alkyl)-, —O—, —C(═O), and —S(O)₀₋₂—;

A is:

(i) —(Y^{A1})ₙ—Y^{A2}, wherein:
n is 0 or 1;
Y^{A1} is C₁₋₆ alkylene, which is optionally substituted with from 1-6 R^{a}; and
Y^{A2} is:
   (a) C₃₋₂₀ cycloalkyl, which is optionally substituted with from 1-4 R^{b},
   (b) C₆₋₂₀ aryl, which is optionally substituted with from 1-4 R^{c};
   (c) heteroaryl including from 5-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; or
   (d) heterocyclyl including from 3-16 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},

OR

(ii) —Z¹—Z²—Z³, wherein:
Z¹ is C₁₋₃ alkylene, which is optionally substituted with from 1-4 R^{a};
Z² is —N(H)—, —N(R^{d})—, —O—, or —S—; and
Z³ is C₂₋₇ alkyl, which is optionally substituted with from 1-4 R^{a};

OR

(iii) C₁₋₁₀ alkyl, which is optionally substituted with from 1-6 independently selected R^{a},

OR

### B

W is selected from the group consisting of:

(a) C₇₋₂₀ bicyclic or polycyclic aryl, which is optionally substituted with from 1-4 R^{c}; and

(b) bicyclic or polycyclic heteroaryl including from 7-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c};

each occurrence of R¹ is independently selected from the group consisting of
H;
halo;
cyano;
C₁₋₆ alkyl optionally substituted with 1-2 R^{a};
C₂₋₆ alkenyl optionally substituted with 1-2 R^{a};
C₂₋₆ alkynyl optionally substituted with 1-2 R^{a};
C₁₋₄ haloalkyl;
C₁₋₄ alkoxy;
C₁₋₄ haloalkoxy;
-L³-L⁴-Rⁱ;
—S(O)₁₋₂(C₁₋₄ alkyl),
—S(O)(═NH)(C₁₋₄ alkyl),
SF₅,
—NR^{e}R^{f},
—OH,
oxo,
—S(O)₁₋₂(NR′R″),
—C₁₋₄ thioalkoxy,
—NO₂,
—C(═O)(C₁₋₄ alkyl),
—C(═O)O(C₁₋₄ alkyl),
—C(═O)OH, and
—C(═O)N(R′)(R″);

or a pair of R¹ on adjacent atoms, taken together with the atoms connecting them, form a ring including from 3-10 ring atoms, wherein from 0-2 ring atoms are heteroatoms each independently selected from the group consisting of N, N(H), N(R^{d}), O, and S(O)₀₋₂; and wherein the ring is optionally substituted with from 1-4 substituents each independently selected from C₁₋₆ alkyl, halo, C₁₋₆ haloalkyl, —OH, NR^{e}R^{f}, C₁₋₆ alkoxy, and C₁₋₆ haloalkoxy,

each occurrence of R² is independently selected from the group consisting of:
(i) C₁₋₆ alkyl, which is optionally substituted with from 1-2 independently selected R^{a};
(ii) C₃₋₆ cycloalkyl;
(iii) heterocyclyl including from 3-10 ring atoms, wherein from 1-3 ring atoms are heteroatoms, each independently selected from the group consisting of N, N(H), N(R^{d}), O, and S(O)₀₋₂;
(iv) C₆₋₁₀ aryl;
(v) heteroaryl including from 5-10 ring atoms, wherein from 1-3 ring atoms are heteroatoms, each independently selected from the group consisting of N, N(H), N(R^{d}), O, and S(O)₀₋₂;
(vi) —C(O)(C₁₋₄ alkyl);
(vii) —C(O)O(C₁₋₄ alkyl);
(viii) —CON(R′)(R″);
(ix) —S(O)₁₋₂(NR′R″);
(x) —S(O)₁₋₂(C₁₋₄ alkyl);
(xi) —OH;
(xii) C₁₋₄ alkoxy; and
(xiii) H;

or a pair of R¹ and R² on adjacent atoms, taken together with the atoms connecting them, form a ring including from 3-10 ring atoms, wherein from 0-2 ring atoms (in addition to the nitrogen atom to which the R² is attached) are heteroatoms each independently selected from the group consisting of N, N(H), N(R^{d}), O, and S(O)₀₋₂; and wherein the ring is optionally substituted with from 1-4 substituents each independently selected from C₁₋₆ alkyl, halo, C₁₋₆ haloalkyl, —OH, NR^{e}R^{f}, C₁₋₆ alkoxy, and C₁₋₆ haloalkoxy,

each occurrence of R³ is independently selected from H; C₁₋₆ alkyl optionally substituted with from 1-6 independently selected R^{a}; C₁₋₄ haloalkyl; —OH; —F; —Cl; —Br; —NR^{e}R^{f}; C₁₋₄ alkoxy; C₁₋₄ haloalkoxy; —C(═O)(C₁₋₄ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and C₃₋₆ cycloalkyl optionally substituted with from 1-4 independently selected C₁₋₄ alkyl; or

two R³ on the same carbon combine to form an oxo; or

a pair of R³, taken together with the atom(s) connecting them, form a ring including from 3-10 ring atoms, wherein from 0-2 ring atoms are heteroatoms each independently selected from the group consisting of N, N(H), N(R^{d}), O, and S(O)₀₋₂; and wherein the ring is optionally substituted with from 1-4 substituents each independently selected from C₁₋₆ alkyl, halo, C₁₋₆ haloalkyl, —OH, NR^{e}R^{f}, C₁₋₆ alkoxy, and C₁₋₆ haloalkoxy; or

a pair of R¹ and R³ on adjacent atoms, taken together with the atoms connecting them, form a ring including from 3-10 ring atoms, wherein from 0-2 ring atoms are heteroatoms each independently selected from the group consisting of N, N(H), N(R^{d}), O, and S(O)₀₋₂; and wherein the ring is optionally substituted with from 1-4 substituents each independently selected from C₁₋₆ alkyl, halo, C₁₋₆ haloalkyl, —OH, NR^{e}R^{f}, C₁₋₆ alkoxy, and C₁₋₆ haloalkoxy; or

or a pair of R² and R³ on adjacent atoms, taken together with the atoms connecting them, form a ring including from 3-10 ring atoms, wherein from 0-2 ring atoms (in addition to the nitrogen atom to which the R² is attached) are heteroatoms each independently selected from the group consisting of N, N(H), N(R^{d}), O, and S(O)₀₋₂; and wherein the ring is optionally substituted with from 1-4 substituents each independently selected from C₁₋₆ alkyl, halo, C₁₋₆ haloalkyl, —OH, NR^{e}R^{f}, C₁₋₆ alkoxy, and C₁₋₆ haloalkoxy;

R⁴ is selected from H and C₁₋₆ alkyl;

R⁵ is selected from H and halo;

R⁶ is selected from H; C₁₋₆ alkyl; —OH; C₁₋₄ alkoxy; C(═O)H; C(═O)(C₁₋₄ alkyl); CN; C₆₋₁₀ aryl optionally substituted with from 1-4 independently selected C₁₋₄ alkyl; and heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 independently selected C₁₋₄ alkyl;

each occurrence of R^{d} is independently selected from the group consisting of:

(a) halo; (b) cyano; (c) C₁₋₁₀ alkyl which is optionally substituted with from 1-6 independently selected R^{a}; (d) C₂₋₆ alkenyl; (e) C₂₋₆ alkynyl; (f) C₃₋₆ cycloalkyl; (g) C₁₋₄ alkoxy; (h) C₁₋₄ haloalkoxy; (i) —S(O)₁₋₂(C₁₋₄ alkyl); (j) —NR^{e}R^{f}; (k) —OH; (l) —S(O)₁₋₂(NR′R″); (m) —C₁₋₄ thioalkoxy; (n) —NO₂; (o) —C(═O)(C₁₋₄ alkyl); (p) —C(═O)O(C₁₋₄ alkyl); (q) —C(═O)OH; (r) —C(═O)N(R′)(R″); and (s) oxo;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl optionally substituted with from 1-4 independently selected C₁₋₄ alkyl;

each occurrence of R^{b} is independently selected from the group consisting of: C₁. alkyl optionally substituted with from 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₄ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of:

(a) halo; (b) cyano; (c) C₁₋₁₀ alkyl which is optionally substituted with from 1-6 independently selected R^{a}; (d) C₂₋₆ alkenyl; (e) C₂₋₆ alkynyl; (g) C₁₋₄ alkoxy; (h) C₁₋₄ haloalkoxy; (i) —S(O)₁₋₂(C₁₋₄ alkyl); (j) —NR^{e}R^{f}; (k) —OH; (l) —S(O)₁₋₂(NR′R″); (m) —C₁₋₄ thioalkoxy; (n) —NO₂; (o) —C(═O)(C₁₋₄ alkyl); (p) —C(═O)O(C₁₋₄ alkyl); (q) —C(═O)OH; (r) —C(═O)N(R′)(R″); (s) -L¹-L²-R^{h}; and (t) oxo;

R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl optionally substituted with from 1-2 substituents each independently selected from halo, OH, C₁₋₄ alkoxy, C₁₋₄ haloalkoxy, and CN; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene optionally substituted with from 1-2 substituents each independently selected from the group consisting of halo, NR^{e}R^{f}, OH, C₁₋₄ alkoxy, and CN;

-L² is —O—, —N(H)—, —S(O)₀₋₂—, or a bond;

R^{h} is selected from:
C₃₋₈ cycloalkyl optionally substituted with from 1-4 substituents independently selected from the group consisting of halo, C₁₋₄ alkyl, hydroxyC₁₋₄ alkyl, and C₁₋₄ haloalkyl (in certain embodiments, it is provided that when R^{h} is C₃₋₆ cycloalkyl optionally substituted with from 1-4 substituents independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo, C₁₋₄ alkyl, hydroxyC₁₋₄ alkyl, and C₁₋₄ haloalkyl;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo, C₁₋₄ alkyl, hydroxyC₁₋₄ alkyl, and C₁₋₄ haloalkyl; and
C₆₋₁₀ aryl, which is optionally substituted with from 1-4 substituents independently selected from the group consisting of halo, C₁₋₄ alkyl, hydroxyC₁₋₄ alkyl, and C₁₋₄ haloalkyl;

-L³ is a bond; C₁₋₃ alkylene optionally substituted with from 1-2 substituents each independently selected from the group consisting of halo, NR^{e}R^{f}, OH, C₁₋₄ alkoxy, and CN; CH═CH; or C≡C;

-L⁴ is —O—, —N(H)—, —S(O)₀₋₂—, or a bond;

R¹ is selected from:
C₃₋₈ cycloalkyl optionally substituted with from 1-4 substituents independently selected from the group consisting of halo, C₁₋₄ alkyl, hydroxyC₁₋₄ alkyl, and C₁₋₄ haloalkyl;
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo, C₁₋₄ alkyl, hydroxyC₁₋₄ alkyl, and C₁₋₄ haloalkyl;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo, C₁₋₄ alkyl, hydroxyC₁₋₄ alkyl, and C₁₋₄ haloalkyl; and
C₆₋₁₀ aryl, which is optionally substituted with from 1-4 substituents independently selected from the group consisting of halo, C₁₋₄ alkyl, hydroxyC₁₋₄ alkyl, and C₁₋₄ haloalkyl; and

each occurrence of R′ and R″ is independently selected from the group consisting of: H, C₁₋₄ alkyl, and C₆₋₁₀ aryl optionally substituted with from 1-2 substituents selected from halo, C₁₋₄ alkyl, and C₁₋₄ haloalkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(C₁₋₆ alkyl), O, and S;

provided that the compound is other than a compound selected from the group consisting of:

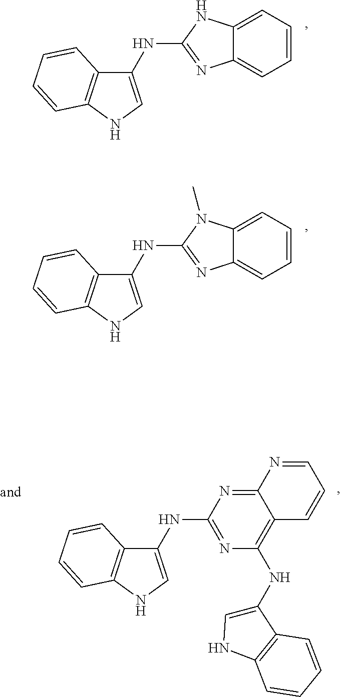 [SMILES: c1ccc2[nH]c(Nc3c[nH]c4ccccc34)nc2c1 ; Cn1c(Nc2c[nH]c3ccccc23)nc2ccccc21 ; c1ccc2c(Nc3nc(Nc4c[nH]c5ccccc45)c4cccnc4n3)c[nH]c2c1]

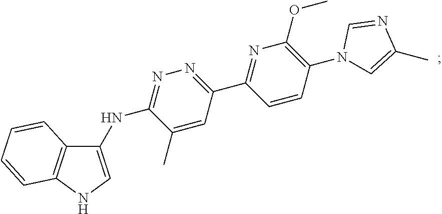 [SMILES: COc1nc(-c2cc(C)c(Nc3c[nH]c4ccccc34)nn2)ccc1-n1cnc(C)c1]

and

further provided that when Z, Y², and Y³ are each CH; Y⁴ is C; Y¹ is CH or C—OH; X¹ is NH; and X² is CH, then W cannot be:
pyrimidinyl substituted with from 1-2 substituents each independently selected from the group consisting of: methyl; —CH₂NH₂; —CH₂N(H)Me; —CH₂CH₂NH₂; —CH₂CH₂N(H)Me; —N(H)Me; —N(H)Et; —N(H)CH₂CH₂NH₂; —N(H)CH₂CH₂OH; —N(H)iPr; —N(H)CH₂CN; cyano; C(═O)OH; and —Cl;
thiazolyl substituted with —CH₂NH₂; or
pyridinyl substituted with from 1-2 substituents each independently from the group consisting of: NH₂; methyl; and Br.

In some embodiments of the compound of Formula (VI), the ring that includes Z, Y, Y², Y³, and Y⁴ is aromatic. In some embodiments of the compound of Formula (VI), X¹ is NR², such as NH. In some embodiments of the compound of Formula (VI), X² is CR⁵, such as CH.

In some embodiments of the compound of Formula (VI), W is defined according to (A).

In some embodiments of the compound of Formula (VI), Q¹ is heteroaryl including from 5-6 ring atoms, wherein from 1-4 ring atoms are heteroatoms, each independently selected from the group consisting of N, N(H), N(R^{d}), O, and S, and wherein the heteroaryl ring is optionally substituted with from 1-4 independently selected R^{a}.

In some embodiments of the compound of Formula (VI), Q² is a bond. In some embodiments of the compound of Formula (VI), A is —(Y^{A1})ₙ—Y^{A2}.

In some embodiments of the compound of Formula (VI), Y^{A2} is C₆₋₁₀ aryl, which is optionally substituted with from 1-3 R^{c}, such as wherein Y^{A2} is C₆ aryl, which is optionally substituted with from 1-3 R^{c}; or wherein Y^{A2} is C₇₋₁₅ bicyclic or tricyclic aryl which is optionally substituted with from 1-3 R^{c}, such as wherein Y^{A2} is naphthyl, tetrahydronaphthyl, indacenyl, or 1′,3′-dihydrospiro[cyclopropane-1,2′-indene] such as

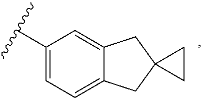 [SMILES: *c1ccc2c(c1)CC1(CC1)C2]

each of which is optionally substituted with from 1-3 R^{c}.

In some embodiments of the compound of Formula (VI), W is defined according to (B). In certain embodiments, W is bicyclic or polycyclic heteroaryl including from 7-ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{e}.

In certain embodiments, W² is selected from the group consisting of:

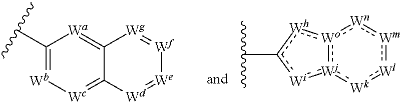

wherein:

W^{a}, W^{b}, W^{c}, W^{d}, W^{e}, W^{f}, and W^{g} are each independently selected from the group consisting of: N, CH, and CR^{c}, provided that from 1-4 of W^{a}-W^{g} is N, and no more than 4 of W^{a}-W^{g} are CR^{c};

W^{h} and Wⁱ are independently selected from the group consisting of N, NH, NR^{d}, O, S, CH, and CR^{c};

W^{j} and W^{o} are independently N or C;

W^{k}, W^{l}, W^{m}, and Wⁿ are independently N, CH, or CR^{c}, provided that:
from 1-4 of W^{h}-W^{o} are heteroatoms,
no more than 4 of W^{h}-W^{o} are CR^{c}, and
when one of W^{h} and Wⁱ is N, the other one of W^{h} and Wⁱ is CH, CR^{c}, O or S;

each [symbol] is independently a single bond or a double bond, provided that the 5-membered ring including Wⁱ, W^{j}, W^{o}, and W^{h} is aromatic, and the 6-membered ring including W^{o}, W^{j}, W^{k}, W^{l}, W^{m}, and Wⁿ is aromatic.

In some embodiments of the compound of Formula (VI),

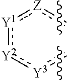

moiety is

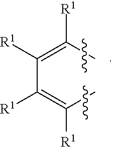

In some other embodiments, from 1-2 of Y¹, Y², and Y³ is independently N or NR² such as N. For example, the

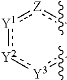

moiety is

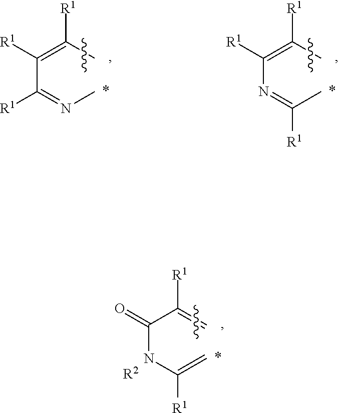

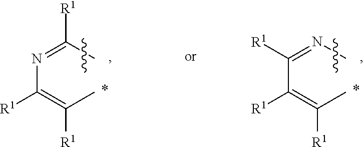

wherein the asterisk denotes point of attachment to Y⁴.

In another aspect, the STING antagonist is a compound selected from the group consisting of compounds in Table 5 and pharmaceutically acceptable salts thereof.

TABLE 5

| Compound | Structure |
|---|---|
| 101 | 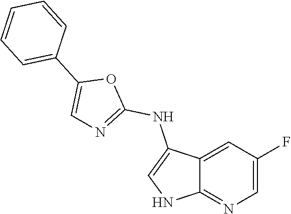 [SMILES: Fc1cnc2[nH]cc(Nc3ncc(-c4ccccc4)o3)c2c1] |
| 102 | 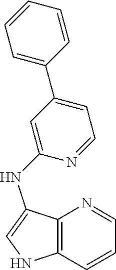 [SMILES: c1ccc(-c2ccnc(Nc3c[nH]c4cccnc34)c2)cc1] |
| 103 | 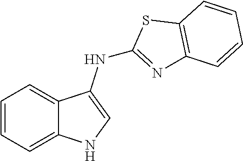 [SMILES: c1ccc2sc(Nc3c[nH]c4ccccc34)nc2c1] |
| 104 | 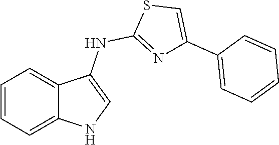 [SMILES: c1ccc(-c2csc(Nc3c[nH]c4ccccc34)n2)cc1] |
| 105 | 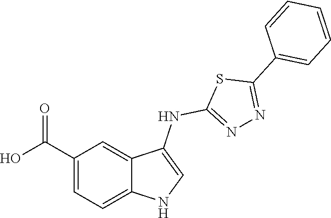 [SMILES: O=C(O)c1ccc2[nH]cc(Nc3nnc(-c4ccccc4)s3)c2c1] |
| 106 | 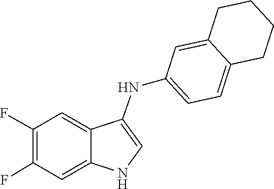 [SMILES: Fc1cc2[nH]cc(Nc3ccc4c(c3)CCCC4)c2cc1F] |
| 107 | 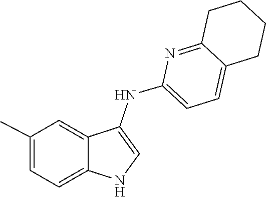 [SMILES: Cc1ccc2[nH]cc(Nc3ccc4c(n3)CCCC4)c2c1] |
| 108 | 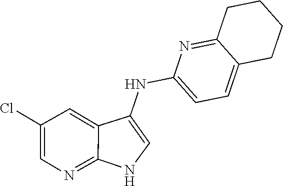 [SMILES: Clc1cnc2[nH]cc(Nc3ccc4c(n3)CCCC4)c2c1] |
| 109 | 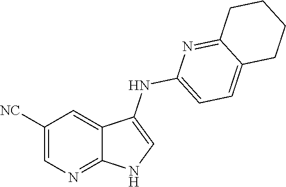 [SMILES: N#Cc1cnc2[nH]cc(Nc3ccc4c(n3)CCCC4)c2c1] |
| 110 | 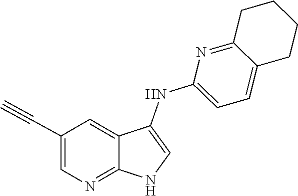 [SMILES: C#Cc1cnc2[nH]cc(Nc3ccc4c(n3)CCCC4)c2c1] |
| 111 | 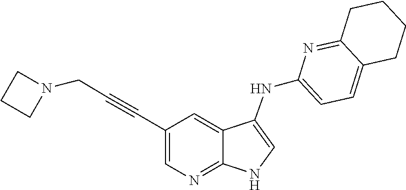 [SMILES: C(#Cc1cnc2[nH]cc(Nc3ccc4c(n3)CCCC4)c2c1)CN1CCC1] |
| 112 | 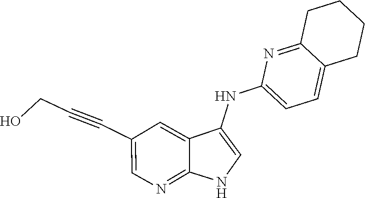 [SMILES: OCC#Cc1cnc2[nH]cc(Nc3ccc4c(n3)CCCC4)c2c1] |
| 113 | 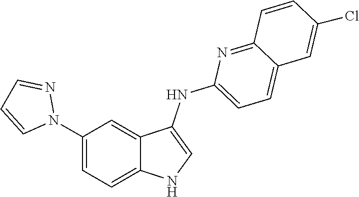 [SMILES: Clc1ccc2nc(Nc3c[nH]c4ccc(-n5cccn5)cc34)ccc2c1] |
| 114 | 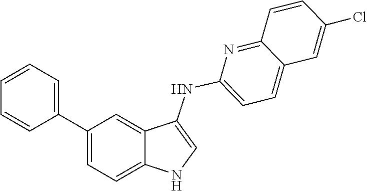 [SMILES: Clc1ccc2nc(Nc3c[nH]c4ccc(-c5ccccc5)cc34)ccc2c1] |
| 115 | 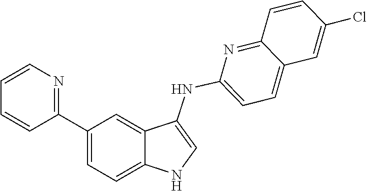 [SMILES: Clc1ccc2nc(Nc3c[nH]c4ccc(-c5ccccn5)cc34)ccc2c1] |
| 116 | 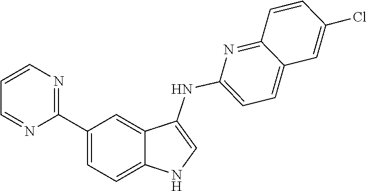 [SMILES: Clc1ccc2nc(Nc3c[nH]c4ccc(-c5ncccn5)cc34)ccc2c1] |
| 117 | 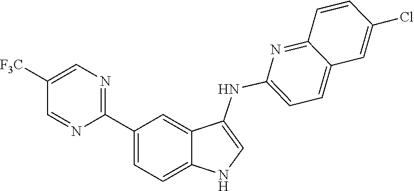 [SMILES: FC(F)(F)c1cnc(-c2ccc3[nH]cc(Nc4ccc5cc(Cl)ccc5n4)c3c2)nc1] |
| 118 | 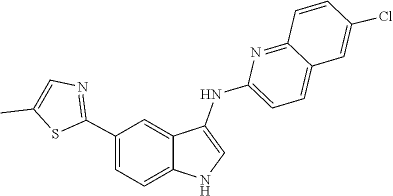 [SMILES: Cc1cnc(-c2ccc3[nH]cc(Nc4ccc5cc(Cl)ccc5n4)c3c2)s1] |
| 119 | 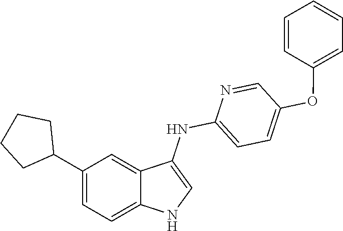 [SMILES: c1ccc(Oc2ccc(Nc3c[nH]c4ccc(C5CCCC5)cc34)nc2)cc1] |
| 120 | 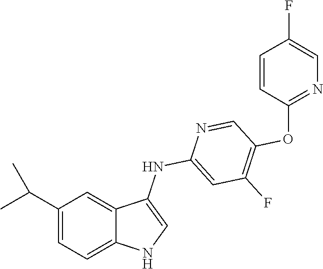 [SMILES: CC(C)c1ccc2[nH]cc(Nc3cc(F)c(Oc4ccc(F)cn4)cn3)c2c1] |
| 121 | 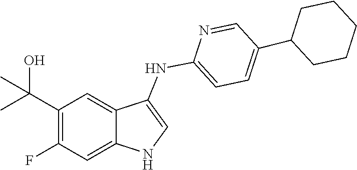 [SMILES: CC(C)(O)c1cc2c(Nc3ccc(C4CCCCC4)cn3)c[nH]c2cc1F] |
| 122 | 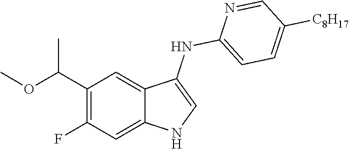 |
| 123 | 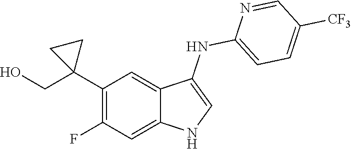 [SMILES: OCC1(c2cc3c(Nc4ccc(C(F)(F)F)cn4)c[nH]c3cc2F)CC1] |
| 124 | 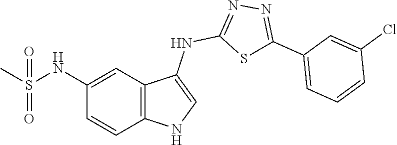 [SMILES: CS(=O)(=O)Nc1ccc2[nH]cc(Nc3nnc(-c4cccc(Cl)c4)s3)c2c1] |
| 125 | 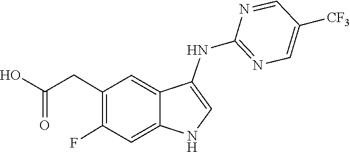 [SMILES: O=C(O)Cc1cc2c(Nc3ncc(C(F)(F)F)cn3)c[nH]c2cc1F] |
| 126 | 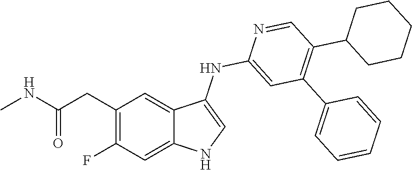 [SMILES: CNC(=O)Cc1cc2c(Nc3cc(-c4ccccc4)c(C4CCCCC4)cn3)c[nH]c2cc1F] |
| 127 | 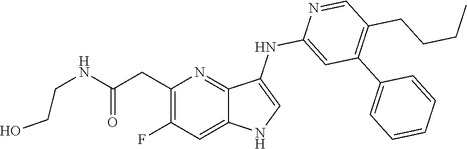 [SMILES: CCCCc1cnc(Nc2c[nH]c3cc(F)c(CC(=O)NCCO)nc23)cc1-c1ccccc1] |
| 128 | 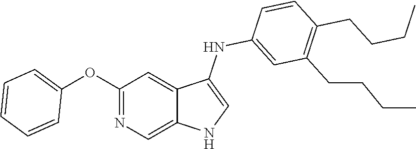 [SMILES: CCCCc1ccc(Nc2c[nH]c3cnc(Oc4ccccc4)cc23)cc1CCCC] |
| 129 | 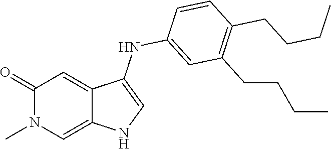 [SMILES: CCCCc1ccc(Nc2c[nH]c3cn(C)c(=O)cc23)cc1CCCC] |
| 130 | 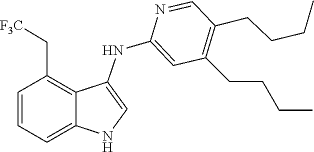 [SMILES: CCCCc1cnc(Nc2c[nH]c3cccc(CC(F)(F)F)c23)cc1CCCC] |
| 131 | 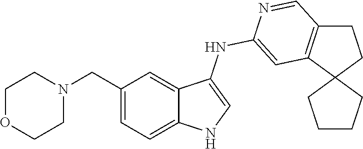 [SMILES: c1cc2[nH]cc(Nc3cc4c(cn3)CCC43CCCC3)c2cc1CN1CCOCC1] |
| 132 | 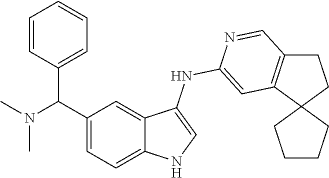 [SMILES: CN(C)C(c1ccccc1)c1ccc2[nH]cc(Nc3cc4c(cn3)CCC43CCCC3)c2c1] |
| 133 | 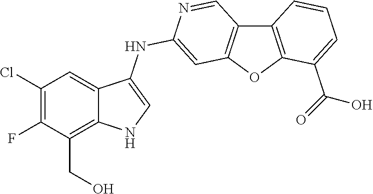 [SMILES: O=C(O)c1cccc2c1oc1cc(Nc3c[nH]c4c(CO)c(F)c(Cl)cc34)ncc12] |
| 134 | 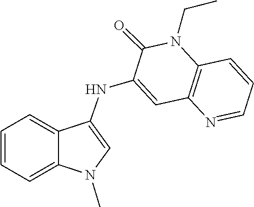 [SMILES: CCn1c(=O)c(Nc2cn(C)c3ccccc23)cc2ncccc21] |
| 135 | 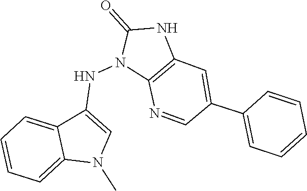 [SMILES: Cn1cc(Nn2c(=O)[nH]c3cc(-c4ccccc4)cnc32)c2ccccc21] |
| 136 | 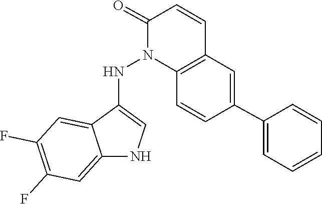 [SMILES: O=c1ccc2cc(-c3ccccc3)ccc2n1Nc1c[nH]c2cc(F)c(F)cc12] |
| 137 | 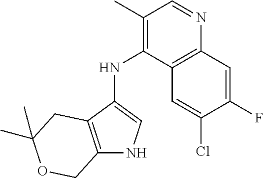 [SMILES: Cc1cnc2cc(F)c(Cl)cc2c1Nc1c[nH]c2c1CC(C)(C)OC2] |
| 138 | 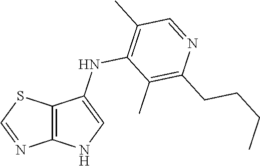 [SMILES: CCCCc1ncc(C)c(Nc2c[nH]c3ncsc23)c1C] |
| 139 | 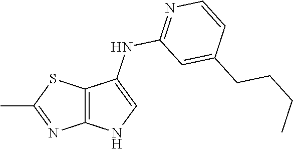 [SMILES: CCCCc1ccnc(Nc2c[nH]c3nc(C)sc23)c1] |
| 140 | 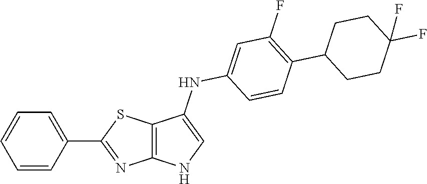 [SMILES: Fc1cc(Nc2c[nH]c3nc(-c4ccccc4)sc23)ccc1C1CCC(F)(F)CC1] |
| 141 | 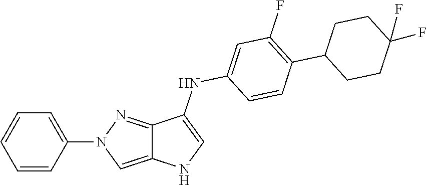 [SMILES: Fc1cc(Nc2c[nH]c3cn(-c4ccccc4)nc23)ccc1C1CCC(F)(F)CC1] |
| 142 | 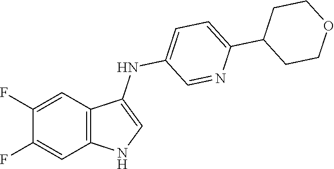 [SMILES: Fc1cc2[nH]cc(Nc3ccc(C4CCOCC4)nc3)c2cc1F] |
| 143 | 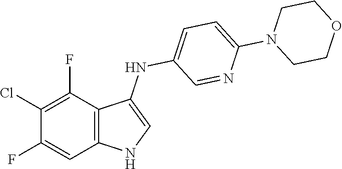 [SMILES: Fc1cc2[nH]cc(Nc3ccc(N4CCOCC4)nc3)c2c(F)c1Cl] |
| 144 | 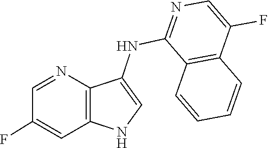 [SMILES: Fc1cnc2c(Nc3ncc(F)c4ccccc34)c[nH]c2c1] |
| 145 | 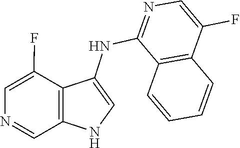 [SMILES: Fc1cnc(Nc2c[nH]c3cncc(F)c23)c2ccccc12] |
| 146 | 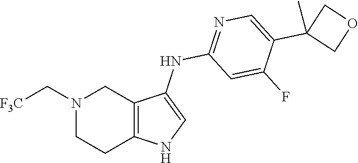 [SMILES: CC1(c2cnc(Nc3c[nH]c4c3CN(CC(F)(F)F)CC4)cc2F)COC1] |
| 147 | 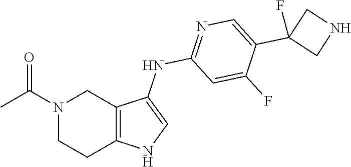 [SMILES: CC(=O)N1CCc2[nH]cc(Nc3cc(F)c(C4(F)CNC4)cn3)c2C1] |
| 148 | 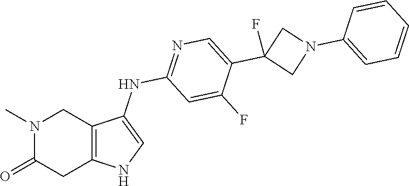 [SMILES: CN1Cc2c(Nc3cc(F)c(C4(F)CN(c5ccccc5)C4)cn3)c[nH]c2CC1=O] |
| 149 | 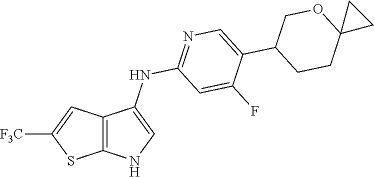 [SMILES: Fc1cc(Nc2c[nH]c3sc(C(F)(F)F)cc23)ncc1C1CCC2(CC2)OC1] |
| 150 | 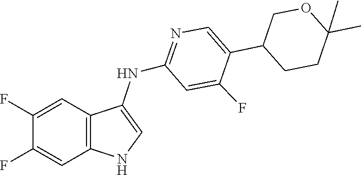 [SMILES: CC1(C)CCC(c2cnc(Nc3c[nH]c4cc(F)c(F)cc34)cc2F)CO1] |
| 151 | 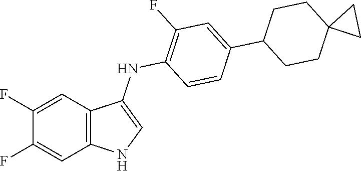 [SMILES: Fc1cc2[nH]cc(Nc3ccc(C4CCC5(CC4)CC5)cc3F)c2cc1F] |
| 152 | 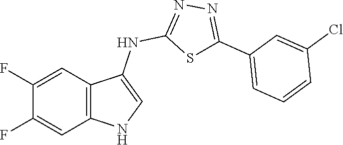 [SMILES: Fc1cc2[nH]cc(Nc3nnc(-c4cccc(Cl)c4)s3)c2cc1F] |
| 153 | 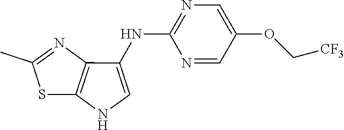 [SMILES: Cc1nc2c(Nc3ncc(OCC(F)(F)F)cn3)c[nH]c2s1] |
| 154 | 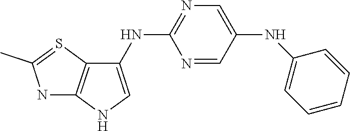 |
| 155 | 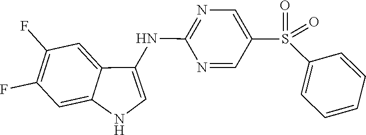 [SMILES: O=S(=O)(c1ccccc1)c1cnc(Nc2c[nH]c3cc(F)c(F)cc23)nc1] |
| 156 | 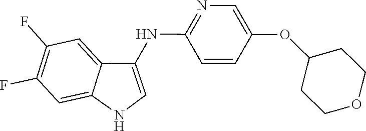 [SMILES: Fc1cc2[nH]cc(Nc3ccc(OC4CCOCC4)cn3)c2cc1F] |
| 157 | 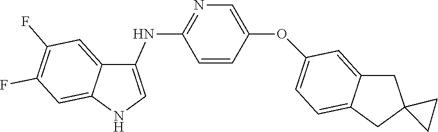 [SMILES: Fc1cc2[nH]cc(Nc3ccc(Oc4ccc5c(c4)CC4(CC4)C5)cn3)c2cc1F] |
| 158 | 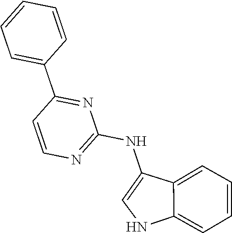 [SMILES: c1ccc(-c2ccnc(Nc3c[nH]c4ccccc34)n2)cc1] |
| 159 | 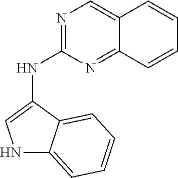 [SMILES: c1ccc2nc(Nc3c[nH]c4ccccc34)ncc2c1] |
| 160 | 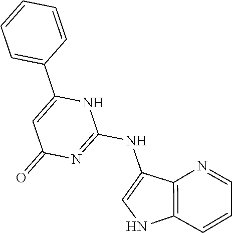 [SMILES: O=c1cc(-c2ccccc2)[nH]c(Nc2c[nH]c3cccnc23)n1] |
| 161 | 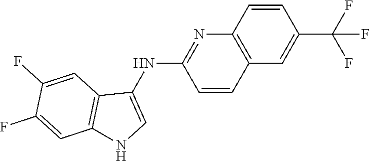 [SMILES: Fc1cc2[nH]cc(Nc3ccc4cc(C(F)(F)F)ccc4n3)c2cc1F] |
| 162 | 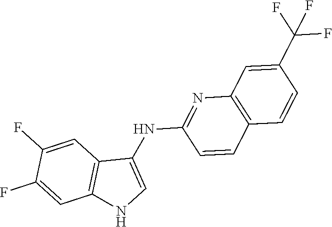 [SMILES: Fc1cc2[nH]cc(Nc3ccc4ccc(C(F)(F)F)cc4n3)c2cc1F] |
| 163 | 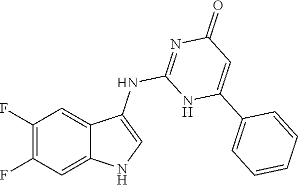 [SMILES: O=c1cc(-c2ccccc2)[nH]c(Nc2c[nH]c3cc(F)c(F)cc23)n1] |
| 164 | 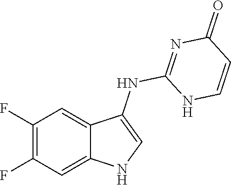 [SMILES: O=c1cc[nH]c(Nc2c[nH]c3cc(F)c(F)cc23)n1] |
| 165 | 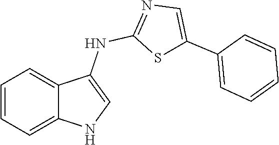 [SMILES: c1ccc(-c2cnc(Nc3c[nH]c4ccccc34)s2)cc1] |
| 166 | 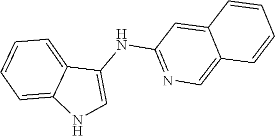 [SMILES: c1ccc2cc(Nc3c[nH]c4ccccc34)ncc2c1] |
| 167 | 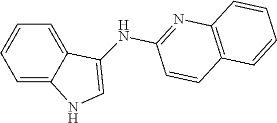 [SMILES: c1ccc2nc(Nc3c[nH]c4ccccc34)ccc2c1] |
| 168 | 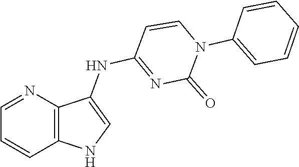 [SMILES: O=c1nc(Nc2c[nH]c3cccnc23)ccn1-c1ccccc1] |
| 169 | 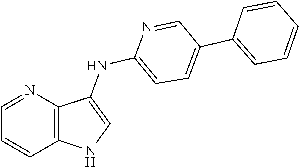 [SMILES: c1ccc(-c2ccc(Nc3c[nH]c4cccnc34)nc2)cc1] |
| 170 | 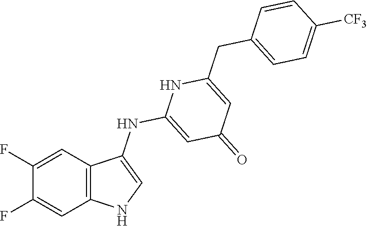 [SMILES: O=c1cc(Cc2ccc(C(F)(F)F)cc2)[nH]c(Nc2c[nH]c3cc(F)c(F)cc23)c1] |
| 171 | 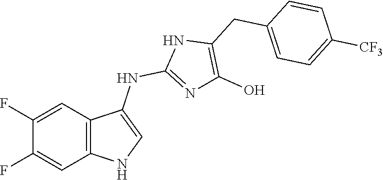 [SMILES: Oc1nc(Nc2c[nH]c3cc(F)c(F)cc23)[nH]c1Cc1ccc(C(F)(F)F)cc1] |
| 172 | 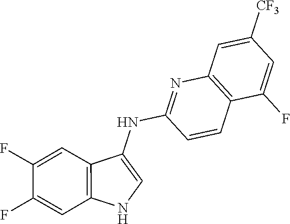 [SMILES: Fc1cc2[nH]cc(Nc3ccc4c(F)cc(C(F)(F)F)cc4n3)c2cc1F] |
| 173 | 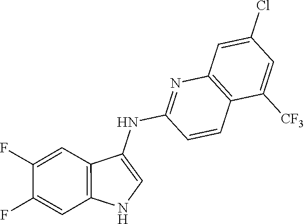 [SMILES: Fc1cc2[nH]cc(Nc3ccc4c(C(F)(F)F)cc(Cl)cc4n3)c2cc1F] |
| 174 | 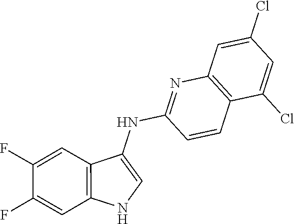 [SMILES: Fc1cc2[nH]cc(Nc3ccc4c(Cl)cc(Cl)cc4n3)c2cc1F] |
| 175 | 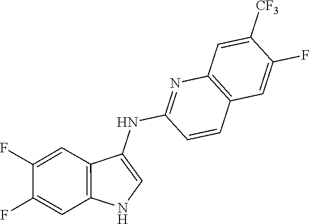 [SMILES: Fc1cc2[nH]cc(Nc3ccc4cc(F)c(C(F)(F)F)cc4n3)c2cc1F] |
| 176 | 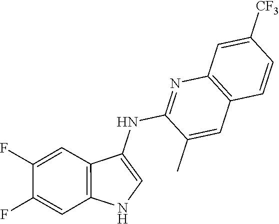 [SMILES: Cc1cc2ccc(C(F)(F)F)cc2nc1Nc1c[nH]c2cc(F)c(F)cc12] |
| 177 | 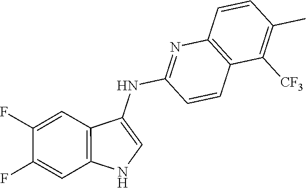 [SMILES: Cc1ccc2nc(Nc3c[nH]c4cc(F)c(F)cc34)ccc2c1C(F)(F)F] |
| 178 | 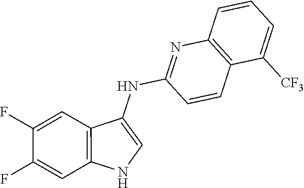 [SMILES: Fc1cc2[nH]cc(Nc3ccc4c(C(F)(F)F)cccc4n3)c2cc1F] |
| 179 | 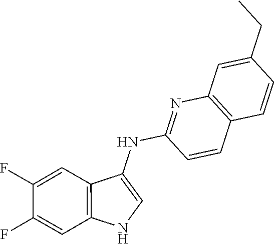 [SMILES: CCc1ccc2ccc(Nc3c[nH]c4cc(F)c(F)cc34)nc2c1] |
| 180 | 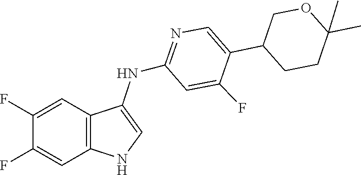 [SMILES: CC1(C)CCC(c2cnc(Nc3c[nH]c4cc(F)c(F)cc34)cc2F)CO1] |
| 181 | 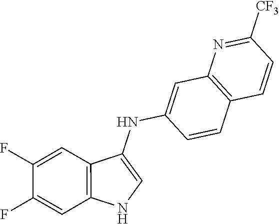 [SMILES: Fc1cc2[nH]cc(Nc3ccc4ccc(C(F)(F)F)nc4c3)c2cc1F] |
| 182 | 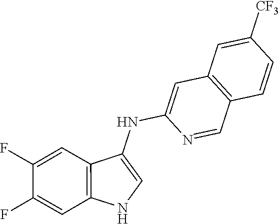 [SMILES: Fc1cc2[nH]cc(Nc3cc4cc(C(F)(F)F)ccc4cn3)c2cc1F] |
| 183 | 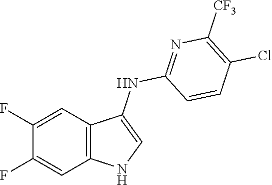 [SMILES: Fc1cc2[nH]cc(Nc3ccc(Cl)c(C(F)(F)F)n3)c2cc1F] |
| 184 | 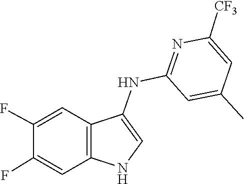 [SMILES: Cc1cc(Nc2c[nH]c3cc(F)c(F)cc23)nc(C(F)(F)F)c1] |
| 185 | 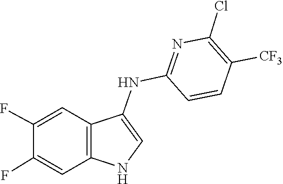 [SMILES: Fc1cc2[nH]cc(Nc3ccc(C(F)(F)F)c(Cl)n3)c2cc1F] |
| 186 | 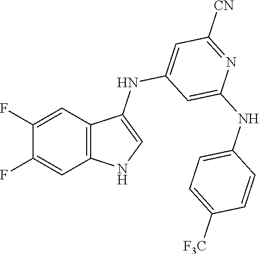 [SMILES: N#Cc1cc(Nc2c[nH]c3cc(F)c(F)cc23)cc(Nc2ccc(C(F)(F)F)cc2)n1] |
| 187 | 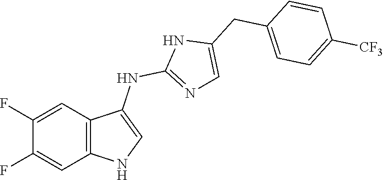 [SMILES: Fc1cc2[nH]cc(Nc3ncc(Cc4ccc(C(F)(F)F)cc4)[nH]3)c2cc1F] |
| 188 | 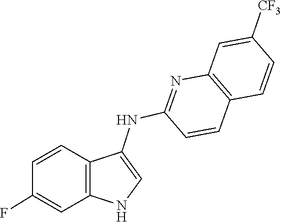 [SMILES: Fc1ccc2c(Nc3ccc4ccc(C(F)(F)F)cc4n3)c[nH]c2c1] |
| 189 | 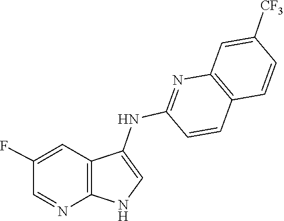 [SMILES: Fc1cnc2[nH]cc(Nc3ccc4ccc(C(F)(F)F)cc4n3)c2c1] |
| 190 | 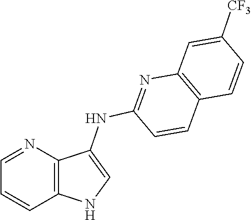 [SMILES: FC(F)(F)c1ccc2ccc(Nc3c[nH]c4cccnc34)nc2c1] |
| 191 | 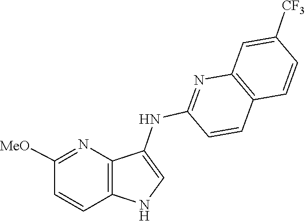 [SMILES: COc1ccc2[nH]cc(Nc3ccc4ccc(C(F)(F)F)cc4n3)c2n1] |

Compounds of Formula (VI) and Table 5, and methods of making and using the same are further described in PCT/US2020/035249, filed on May 29, 2020; and U.S. Provisional 62/854,288, filed on May 29, 2019, each of which is incorporated herein by reference in its entirety.

In another aspect, the STING antagonist is a compound of Formula (VII):

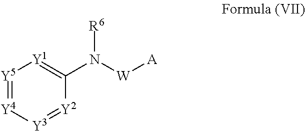

or a pharmaceutically acceptable salt thereof or a tautomer thereof,
wherein:
each of Y¹, Y², Y³, Y⁴, and Y⁵ is independently selected from the group consisting of N and CR¹;
W-A is defined according to (A) or (B) below:

### A

W is selected from the group consisting of:
(a) *C(═O)NR^{N}, *C(═S)NR^{N}, *C(═NR^{N})NR^{N} (e.g., *C(═NCN)NR^{N}), *C(═CNO₂)NR^{N}
(b) *S(O)₁₋₂NR^{N};
(c)

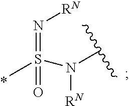

(d)

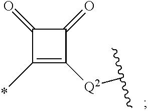

(e) *Q¹-Q²;
wherein the asterisk denotes point of attachment to NR⁶
   Q¹ is selected from the group consisting of:
(a) phenylene optionally substituted with from 1-2 independently selected Rai; and
(b) heteroarylene including from 5-6 ring atoms, wherein from 1-4 ring atoms are heteroatoms, each independently selected from the group consisting of N, N(H), N(R^{d}), O, and S(O)₀₋₂, and wherein the heteroarylene ring is optionally substituted with from 1-4 independently selected R^{q1};
   Q² is selected from the group consisting of: a bond, NR^{N}, —S(O)₀₋₂—, —O—, and —C(═O)—;

### A is:

(i) —Y^{A1}—Y^{A2}, wherein:
Y^{A1} is a bond; or
Y^{A1} is C₁₋₆ alkylene, which is optionally substituted with from 1-6 substituents each independently selected from the group consisting of:
   R^{a};
   C₆₋₁₀ aryl optionally substituted with 1-4 independently selected C₁₋₄ alkyl; and
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected C₁₋₄ alkyl; or
Y^{A1} is —Y^{A3}—Y^{A4}—Y^{A5} which is connected to W via Y^{A3} wherein:
   Y^{A3} is a C₁₋₃ alkylene optionally substituted with from 1-2 independently selected R^{a};
   Y^{A4} is —O—, —NH—, or —S—; and
   Y^{A5} is a bond or C₁₋₃ alkylene which is optionally substituted with from 1-2 independently selected R^{a}; and
Y^{A2} is:
(a) C₃₋₂₀ cycloalkyl, which is optionally substituted with from 1-4 R^{b},
(b) C₆₋₂₀ aryl, which is optionally substituted with from 1-4 R^{c},
(c) heteroaryl including from 5-20 ring atoms, wherein from 1-3 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; or
(d) heterocyclyl including from 3-16 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},

### OR

(ii) —Z¹—Z²—Z³, wherein:
Z¹ is C₁₋₃ alkylene, which is optionally substituted with from 1-4 R^{a};
Z² is —N(H)—, —N(R^{d})—, —O—, or —S—; and
Z³ is C₂₋₇ alkyl, which is optionally substituted with from 1-4 R^{a};

### OR

(iii) C₁₋₂₀ alkyl, which is optionally substituted with from 1-6 independently selected R^{a},

### OR

### B

W is selected from the group consisting of:

(a) C₈₋₂₀ bicyclic or polycyclic arylene, which is optionally substituted with from 1-4 R^{c}; and

(b) bicyclic or polycyclic heteroarylene including from 8-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{e};

A is as defined for (A), or A is H;

each occurrence of R¹ is independently selected from the group consisting of
H;
halo;
cyano;
C₁₋₆ alkyl optionally substituted with 1-2 R^{a};
C₂₋₆ alkenyl;
C₂₋₆ alkynyl;
C₁₋₄ haloalkyl;
C₁₋₄ alkoxy;
C₁₋₄ haloalkoxy;
—S(O)₁₋₂(C₁₋₄ alkyl),
—S(O)(═NH)(C₁₋₄ alkyl),
SF₅,
—NR^{e}R^{f},
—OH,
oxo,
—S(O)₁₋₂(NR′R″),
—C₁₋₄ thioalkoxy,
—NO₂,
—C(═O)(C₁₋₄ alkyl),
—C(═O)O(C₁₋₄ alkyl),
—C(═O)OH,
—C(═O)N(R′)(R″), and
-L³-L⁴-L⁵-Rⁱ;

or a pair of R¹ on adjacent atoms, taken together with the atoms connecting them, form a ring (e.g., aromatic or non-aromatic ring) including from 4-15 ring atoms, wherein from 0-3 ring atoms are heteroatoms each independently selected from the group consisting of N, N(H), N(R^{d}), O, and S(O)₀₋₂; and wherein the ring is optionally substituted with from 1-4 independently selected R²;

each R² is independently selected from the group consisting of:
halo;
cyano;
C₁₋₆ alkyl optionally substituted with 1-2 R^{a};
C₂₋₆ alkenyl;
C₂₋₆ alkynyl;
C₁₋₄ haloalkyl;
C₁₋₄ alkoxy;
C₁₋₄ haloalkoxy;
—S(O)₁₋₂(C₁₋₄ alkyl) optionally substituted with from 1-3 independently selected R^{a},
—S(O)(═NH)(C₁₋₄ alkyl) optionally substituted with from 1-3 independently selected R^{a},
SF₅,
—NR^{e}R^{f},
—OH,
oxo,
—S(O)₁₋₂(NR′R″),
—C₁₋₄ thioalkoxy,
—NO₂,
—C(═O)(C₁₋₄ alkyl) optionally substituted with from 1-3 independently selected R^{a},
—C(═O)O(C₁₋₄ alkyl) optionally substituted with from 1-3 independently selected R^{a},
—C(═O)OH,
—C(═O)N(R′)(R″); and
-L³-L⁴-L⁵-Rⁱ;

R⁶ is selected from H; C₁₋₆ alkyl; —OH; C₁₋₄ alkoxy; C(═O)H; C(═O)(C₁₋₄ alkyl); CN; C₆₋₁₀ aryl optionally substituted with from 1-4 independently selected C₁₋₄ alkyl; and heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 independently selected C₁₋₄ alkyl;

each occurrence of R^{d} is independently selected from the group consisting of:

(a) halo; (b) cyano; (c) C₁₋₁₀ alkyl which is optionally substituted with from 1-6 independently selected R^{a}; (d) C₂₋₆ alkenyl; (e) C₂₋₆ alkynyl; (f) C₃₋₆ cycloalkyl; (g) C₁₋₄ alkoxy; (h) C₁₋₄ haloalkoxy; (i) —S(O)₁₋₂(C₁₋₄ alkyl); (j) —NR^{e}R^{f}; (k) —OH; (l) —S(O)₁₋₂(NR′R″); (m) —C₁₋₄ thioalkoxy; (n) —NO₂; (o) —C(═O)(C₁₋₄ alkyl); (p) —C(═O)O(C₁₋₄ alkyl); (q) —C(═O)OH; (r) —C(═O)N(R′)(R″); and (s) oxo;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —OCON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;

each occurrence of R^{b} is independently selected from the group consisting of: C₁. alkyl optionally substituted with from 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of:

(a) halo; (b) cyano; (c) C₁₋₁₀ alkyl which is optionally substituted with from 1-6 independently selected R^{a}; (d) C₂₋₆ alkenyl; (e) C₂₋₆ alkynyl; (g) C₁₋₄ alkoxy; (h) C₁₋₄ haloalkoxy; (i) —S(O)₁₋₂(C₁₋₄ alkyl) or —S(O)₁₋₂(C₁₋₄ haloalkyl); (j) —NR^{e}R^{f}; (k) —OH; (l) —S(O)₁₋₂(NR′R″); (m) —C₁₋₄ thioalkoxy or —C₁₋₄ thiohaloalkoxy; (n) —NO₂; (o) —C(═O)(C₁₋₁₀ alkyl); (p) —C(═O)O(C₁₋₄ alkyl); (q) —C(═O)OH; (r) —C(═O)N(R′)(R″); (s) -L¹-L²-R^{h}; (t) —SF₅; and (u) azido;

each occurrence of R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; C₁₋₄ alkoxy; and CN;

each occurrence of R^{e} and R^{f} is independently selected from the group consisting of: H; C₁₋₆ alkyl, wherein the C₁₋₆ alkyl is independently selected with from 1-4 substituents each independently selected from halo, CN, C₁₋₄ alkoxy, C₁₋₄ haloalkoxy, NR′R″, and —OH; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —S(O)(═NR′)(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene optionally substituted with oxo;
-L² is —O—, —N(H)—, —S(O)₀₋₂—, or a bond;
R^{h} is selected from:
C₃₋₈ cycloalkyl optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;
   -L³ is a bond or C₁₋₃ alkylene optionally substituted with oxo;
   -L⁴ is a bond; —O—; —N(R^{N})—; —S(O)₀₋₂—; C(═O); —NR^{N}S(O)₀₋₂-; —S(O)₀₋₂NR^{N}—; —NR^{N}S(O)₁₋₂NR^{N}—; —S(═O)(═NR^{N}); —NR^{N}S(═O)(═NR^{N}); —S(═O)(═NR^{N})NR^{N}; NR^{N}S(═O)(═NR^{N})NR^{N}; —NR^{N}C(O)—; —NR^{N}C(O)NR^{N}—; C₃₋₆ cycloalkylene; or heterocyclylene including from 3-8 ring atoms wherein from 1-3 ring atoms are heteroatoms each independently selected from the group consisting of N, NH, N(R^{d}), O, and S(O)₀₋₂;
   -L⁵ is a bond or C₁₋₄ alkylene;
   Rⁱ is selected from:
C₃₋₈ cycloalkyl optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy (in certain embodiments, it is provided that when Rⁱ is C₃₋₆ cycloalkyl optionally substituted with from 1-4 substituents independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;

each occurrence of R^{N} is independently H or R^{d}; and

each occurrence of R′ and R″ is independently selected from the group consisting of: H, C₁₋₄ alkyl, and C₆₋₁₀ aryl optionally substituted with from 1-2 substituents selected from halo, C₁₋₄ alkyl, and C₁₋₄ haloalkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(C₁₋₆ alkyl), O, and S;

provided that when the compound has Formula (VII-a1) wherein R^{2′} is H or R², W-A is defined according to (A), and W is *C(O)NR^{N} (e.g., *C(O)NH), then 1, 2, 3, 4, or of the following provisions apply:

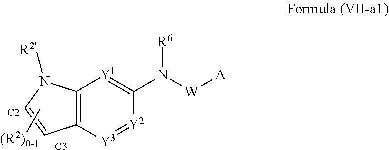

(i) when each of Y¹ and Y² is CH; Y³ is CR¹; R′ is CO₂Me, CO₂Et, CN, or Cl; and R² is absent (i.e., C₂ and C₃ are substituted with H), OR when each of Y¹ and Y² is N; and Y³ is OH or oxo, then A cannot be optionally substituted C₁₋₆ alkyl, such as methyl or butyl, 1,1,3,3-tetramethylbutyl, or optionally substituted C₃ or C₆ cycloalkyl (such as C₁₋₆ alkyl or C₃ or C₆ cycloalkyl optionally substituted with CO₂H, isocyanate, or substituted amino);

(ii) when each of Y¹ and Y² is N; and Y³ is CR¹; then
R¹ cannot be furyl, when W-A is benzyl; and
R¹ cannot be substituted N-linked aniline or chloro when either R^{2′} is methyl or when W-A is phenyl substituted with from 1-2 substituents independently selected from —Cl, —F, —Br, and CF₃;

(iii) when each of Y¹, Y², and; Y³ is CH; R^{2′} is H, R² is present and attached at the C₃-position of the indole ring; and A is phenyl, tolyl, optionally substituted quinazolinyl, optionally substituted pyrazolyl, optionally substituted indolyl, optionally substituted naphthyl, or optionally substituted moropholinyl-phenyl, then R² cannot be oxazolyl, pyridyl, C-linked-2-pyridylethyl, phenyl, cyano, or C(O)NH₂;

(iv) when each of Y¹ and Y³ is CH; Y² is CH or CMe; R^{2′} is H; and R² is absent, then:
R^{h} cannot be a fused tricyclic ring;
Y^{A2} cannot be optionally substituted cyclohexyl, cyclohexenyl, imidazo[1,2-a][1,4]benzodiazepin-4-yl, indenyl, naphthyl, or tetrahydronaphthyl;
Y cannot be alkylene substituted with phenyl;
when Y^{A1} is alkylene, Y^{A2} cannot be phenyl or the following substituted phenyl rings: 4-Br, 2,4-(Cl)₂, 3-propenyl, 2,3-(OMe)₂, and 4-CF₃; and
when Y^{A1} is absent, Y^{A2} cannot be phenyl or the following substituted phenyl rings: 3-NO₂, 4-Br, 2,4-(Cl)₂, 2,3-(OMe)₂, 4-CF₃, 4-CO₂Et, 3-CF₃-4-Cl, 2-Cl-4 CF₃, 2-OEt, 2-OMe-4-NO₂, 3,4-(OMe)₂, 2,4-(Me)₂, 3,4-(Cl)₂, 2,4-(F)₂, 2-Et, 2-F, 2-Me, 2-Br, 2-Cl-4-Br, 2-CF₃, 2,4-(OMe)₂, 2,3-(Me)₂, 3,5-(Cl)₂, 3-CF₃-4-F, 4-iso-propyl, 4-OMe, 4-Cl, 3-F-4-Me, 3-CF₃, 2,5-(OMe)₂, 2-Me-3-Cl, 2,3-(Me)₂, 2,3-(Cl)₂, 4-Bu, 3-OMe, 3-Cl, 4-Me-2-Cl, 3-SMe, 2-CO₂Me, 4-Me-3-Cl, 3,4-(Me)₂, 4-sec-butyl, 2-OMe, 2-Cl, 2,4-(OMe)₂-5-Cl, 4-OEt, 4-acetyl, 2-OMe-5-Me, 2-Me-5-Cl, 3,5-(Me)₂, 3,5-(Cl)₂, 4-NO₂, 4-Br, 4-F, 4-Me, 4-Et, 3-F, 3-Me, 3-acetyl, or 2-Me-5-Cl; and

(v) the compound is other than:

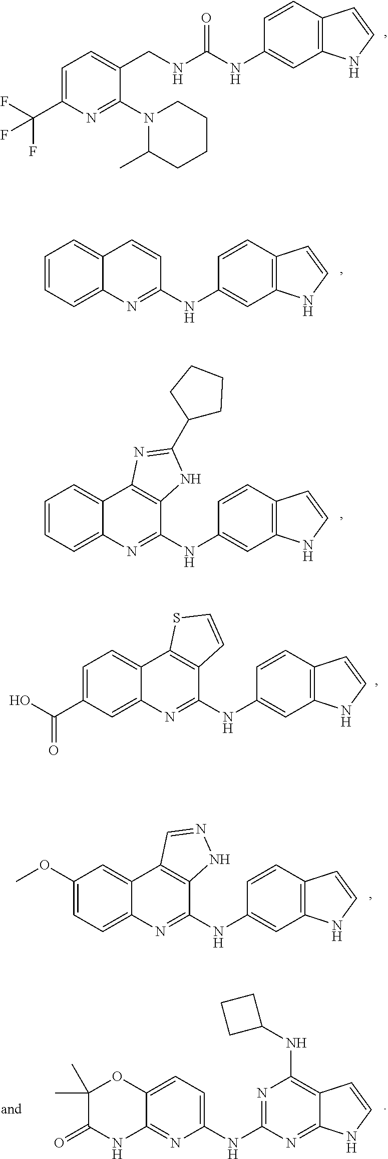 [SMILES: CC1CCCCN1c1nc(C(F)(F)F)ccc1CNC(=O)Nc1ccc2cc[nH]c2c1 ; c1ccc2nc(Nc3ccc4cc[nH]c4c3)ccc2c1 ; c1ccc2c(c1)nc(Nc1ccc3cc[nH]c3c1)c1[nH]c(C3CCCC3)nc12 ; O=C(O)c1ccc2c(c1)nc(Nc1ccc3cc[nH]c3c1)c1ccsc12 ; COc1ccc2nc(Nc3ccc4cc[nH]c4c3)c3[nH]ncc3c2c1 ; CC1(C)Oc2ccc(Nc3nc(NC4CCC4)c4cc[nH]c4n3)nc2NC1=O]

In some embodiments of the compound of Formula (VII), a pair of R¹ on adjacent atoms, taken together with the atoms connecting them, form an aromatic ring including 5 ring atoms, wherein from 1-2 (such as 1 or 2) ring atoms are heteroatoms each independently selected from the group consisting of N, N(H), N(R^{d}), O, and S(O)₀₋₂; and wherein the ring is optionally substituted with from 1-4 independently selected R².

In some embodiments of the compound of Formula (VII), a pair of R¹ on adjacent atoms, taken together with the atoms connecting them, form:

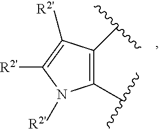

wherein each R^{2′} is independently H or R², such as

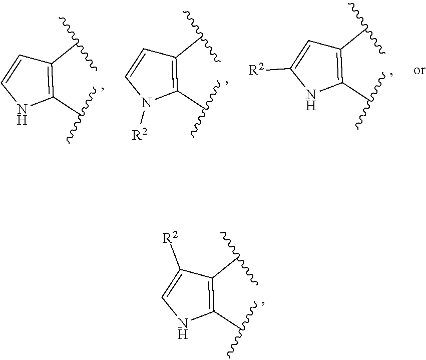

such as

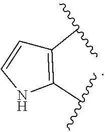 [SMILES: *c1cc[nH]c1*]

In some embodiments of the compound of Formula (VII), the compound has the following formula:

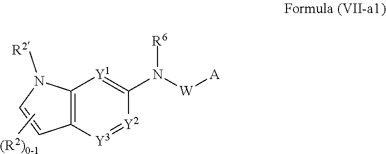

such as,

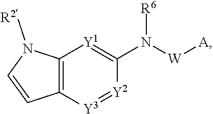

wherein R^{2′} is H or R², such as R^{2′} is H.

In some embodiments of the compound of Formula (VII), the compound has formula

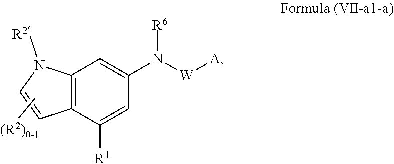

wherein R^{2′} is H or R², such as

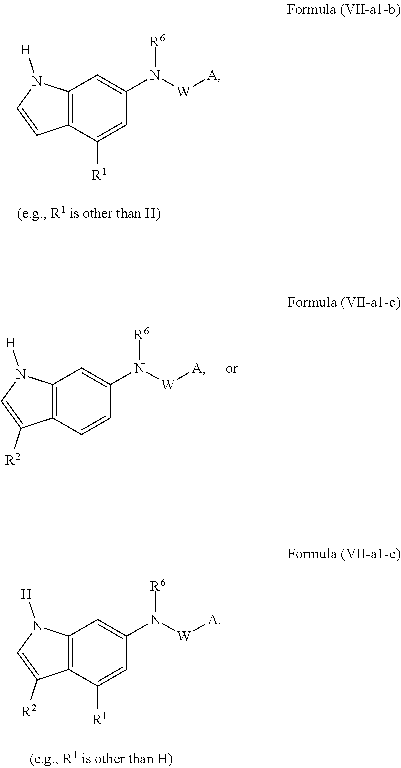

In some embodiments of the compound of Formula (VII), each occurrence of R¹ that is not taken together with the atom to which it is attached in ring formation is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —NR^{e}R^{f}; —OH; oxo; —S(O)₁₋₂(NR′R″); —C(═O)(C₁₋₄ alkyl); —C(═O)O(C₁₋₄ alkyl); —C(═O)OH; —C(═O)N(R′)(R″); and -L³-L⁴-Rⁱ, such as R¹ is halo; cyano; C₁₋₆ alkyl optionally substituted with 1-2 R^{a}; C₁₋₄ haloalkyl; C₁₋₄ alkoxy; or C₁₋₄ haloalkoxy, such as R¹ is halo.

In some embodiments of the compound of Formula (VII), W-A as defined according to (A). In certain of these embodiments, W is *C(═O)NR^{N}, such as *C(═O)NH.

In some embodiments of the compound of Formula (VII), W-A is as defined according to (B).

In some embodiments of the compound of Formula (VII), W is bicyclic heteroarylene including from 8-10 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; and A is H,

such as W is selected from the group consisting of quinolinylene, isoquinolinylene, and quinazolinylene, each of which is optionally substituted with from 1-2 independently selected R^{c}, such as W is

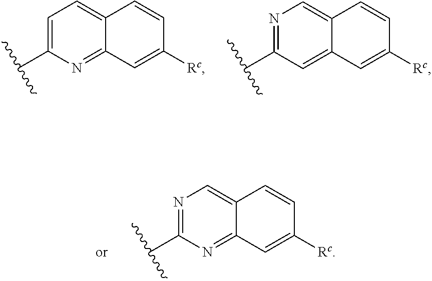

In some embodiments of the compound of Formula (VII), A is —Y^{A1}—Y^{A2}.

In some embodiments of the compound of Formula (VII), Y^{A2} is C₆₋₁₀ aryl, which is optionally substituted with from 1-3 R^{c}.

In some embodiments of the compound of Formula (VII), the compound has one of the following formulae:

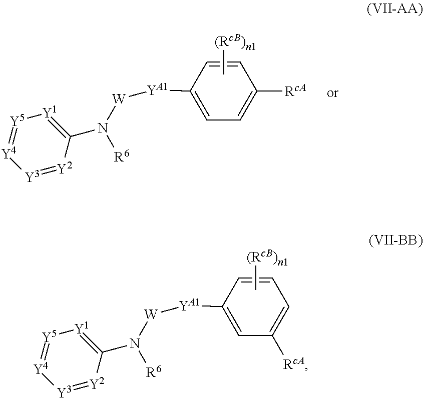

wherein:

n1 is 0, 1, or 2 (such as 0 or 1); each of R^{cA} and R^{cB} is an independently selected R^{c};

W is *C(═O)NR^{N}, such as *C(═O)NH; and

the

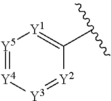

moiety is

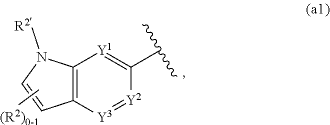

wherein R²′ is H or R².

In some embodiments of the compound of Formula (VII), the

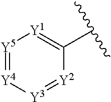

moiety is

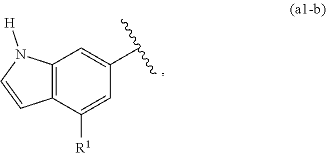

such as (a1-b) wherein R¹ is other than H (e.g., R¹ is halo or cyano).

In some embodiments of the compound of Formula (VII), W is heteroarylene including from 9-10 ring atoms, wherein from 1-2 ring atoms are heteroatoms, each independently selected from the group consisting of N, N(H), N(R^{d}), O, and S(O)₀₋₂, and wherein the heteroaryl ring is optionally substituted with from 1-2 independently selected R^{c}, such as

W is selected from the group consisting of quinolinylene and quinazolinylene, each of which is optionally substituted with from 1-2 independently selected R^{c}, such as:

W is

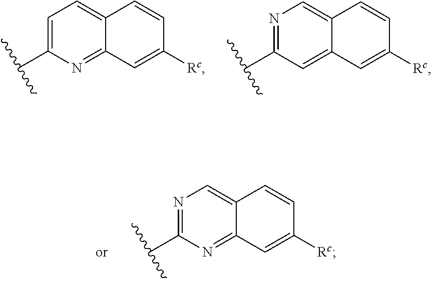

the

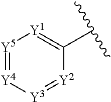

moiety is

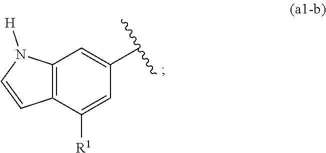

and

A is H, optionally wherein R⁶ is H.

In another aspect, the STING antagonist is a compound selected from the group consisting of compounds in Table 6 and pharmaceutically acceptable salts thereof.

TABLE 6

| Com- | |
|---|---|
| pound | |
| No. | Structure |
| 101 | 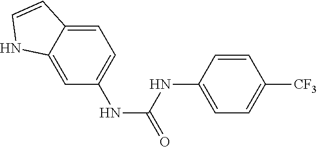 [SMILES: O=C(Nc1ccc(C(F)(F)F)cc1)Nc1ccc2cc[nH]c2c1] |
| 102 | 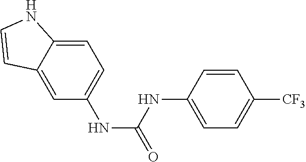 [SMILES: O=C(Nc1ccc(C(F)(F)F)cc1)Nc1ccc2[nH]ccc2c1] |
| 103 | 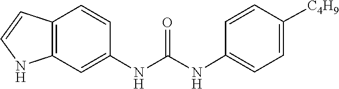 |
| 104 | 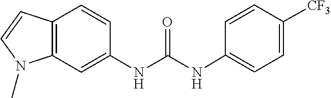 [SMILES: Cn1ccc2ccc(NC(=O)Nc3ccc(C(F)(F)F)cc3)cc21] |
| 105 | 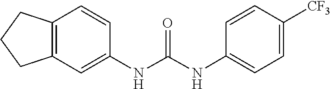 [SMILES: O=C(Nc1ccc(C(F)(F)F)cc1)Nc1ccc2c(c1)CCC2] |
| 106 | 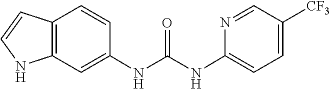 [SMILES: O=C(Nc1ccc2cc[nH]c2c1)Nc1ccc(C(F)(F)F)cn1] |
| 107 | 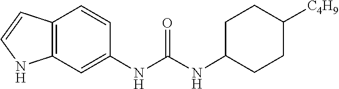 |
| 108 | 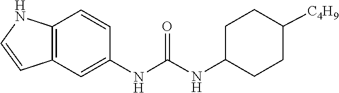 |
| 109 | 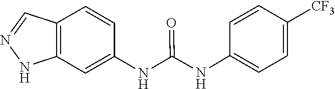 [SMILES: O=C(Nc1ccc(C(F)(F)F)cc1)Nc1ccc2cn[nH]c2c1] |
| 110 | 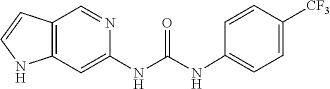 [SMILES: O=C(Nc1ccc(C(F)(F)F)cc1)Nc1cc2[nH]ccc2cn1] |
| 111 | 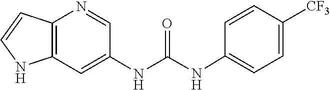 [SMILES: O=C(Nc1ccc(C(F)(F)F)cc1)Nc1cnc2cc[nH]c2c1] |
| 112 | 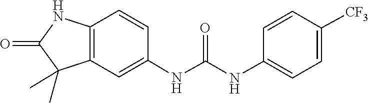 [SMILES: CC1(C)C(=O)Nc2ccc(NC(=O)Nc3ccc(C(F)(F)F)cc3)cc21] |
| 113 | 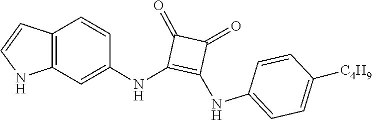 |
| 114 | 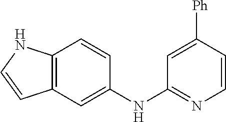 [SMILES: c1ccc(-c2ccnc(Nc3ccc4[nH]ccc4c3)c2)cc1] |
| 115 | 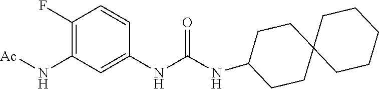 [SMILES: CC(=O)Nc1cc(NC(=O)NC2CCC3(CCCCC3)CC2)ccc1F] |
| 116 | 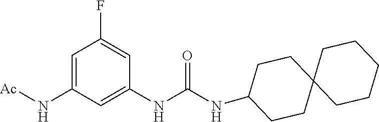 [SMILES: CC(=O)Nc1cc(F)cc(NC(=O)NC2CCC3(CCCCC3)CC2)c1] |
| 117 | 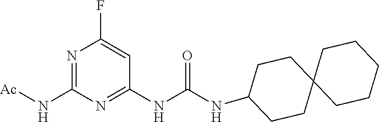 [SMILES: CC(=O)Nc1nc(F)cc(NC(=O)NC2CCC3(CCCCC3)CC2)n1] |
| 118 | 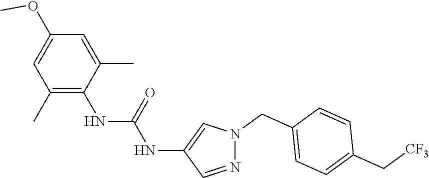 [SMILES: COc1cc(C)c(NC(=O)Nc2cnn(Cc3ccc(CC(F)(F)F)cc3)c2)c(C)c1] |
| 119 | 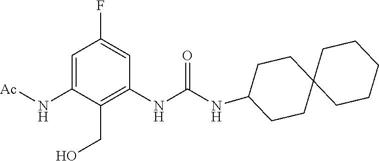 [SMILES: CC(=O)Nc1cc(F)cc(NC(=O)NC2CCC3(CCCCC3)CC2)c1CO] |
| 120 | 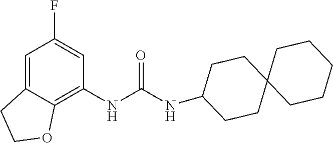 [SMILES: O=C(Nc1cc(F)cc2c1OCC2)NC1CCC2(CCCCC2)CC1] |
| 121 | 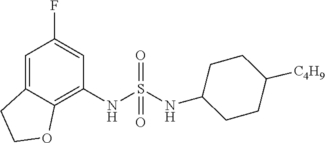 |
| 122 | 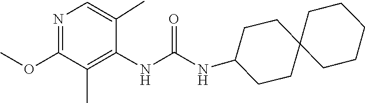 [SMILES: COc1ncc(C)c(NC(=O)NC2CCC3(CCCCC3)CC2)c1C] |
| 123 | 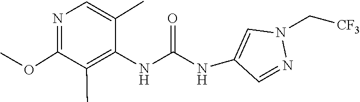 [SMILES: COc1ncc(C)c(NC(=O)Nc2cnn(CC(F)(F)F)c2)c1C] |
| 124 | 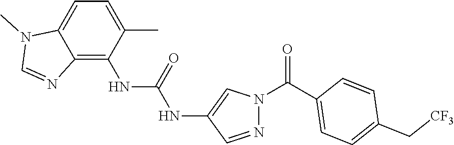 [SMILES: Cc1ccc2c(ncn2C)c1NC(=O)Nc1cnn(C(=O)c2ccc(CC(F)(F)F)cc2)c1] |
| 125 | 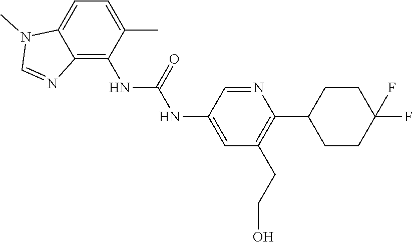 [SMILES: Cc1ccc2c(ncn2C)c1NC(=O)Nc1cnc(C2CCC(F)(F)CC2)c(CCO)c1] |
| 126 | 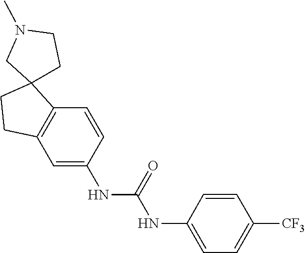 [SMILES: CN1CCC2(CCc3cc(NC(=O)Nc4ccc(C(F)(F)F)cc4)ccc32)C1] |
| 127 | 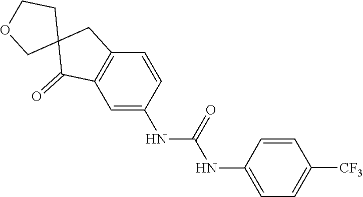 [SMILES: O=C(Nc1ccc(C(F)(F)F)cc1)Nc1ccc2c(c1)C(=O)C1(CCOC1)C2] |
| 128 | 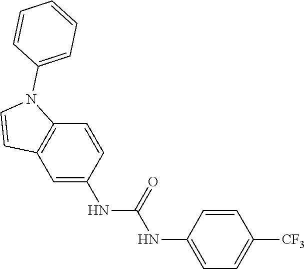 [SMILES: O=C(Nc1ccc(C(F)(F)F)cc1)Nc1ccc2c(ccn2-c2ccccc2)c1] |
| 129 | 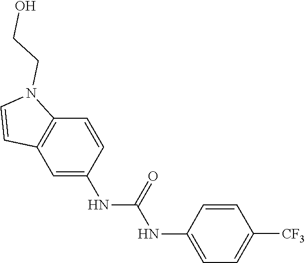 [SMILES: O=C(Nc1ccc(C(F)(F)F)cc1)Nc1ccc2c(ccn2CCO)c1] |
| 130 | 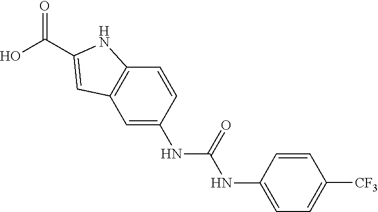 [SMILES: O=C(Nc1ccc(C(F)(F)F)cc1)Nc1ccc2[nH]c(C(=O)O)cc2c1] |
| 131 | 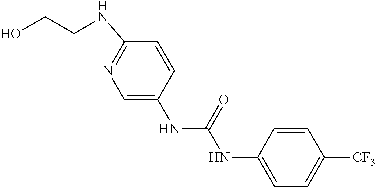 [SMILES: O=C(Nc1ccc(C(F)(F)F)cc1)Nc1ccc(NCCO)nc1] |
| 132 | 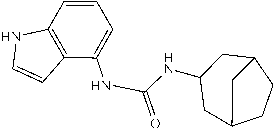 [SMILES: O=C(Nc1cccc2[nH]ccc12)NC1CC2CCC(C2)C1] |
| 133 | 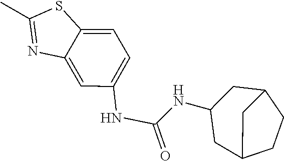 [SMILES: Cc1nc2cc(NC(=O)NC3CC4CCC(C4)C3)ccc2s1] |
| 134 | 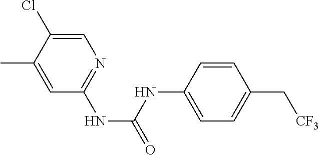 [SMILES: Cc1cc(NC(=O)Nc2ccc(CC(F)(F)F)cc2)ncc1Cl] |
| 135 | 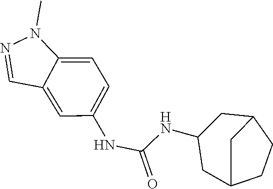 [SMILES: Cn1ncc2cc(NC(=O)NC3CC4CCC(C4)C3)ccc21] |
| 136 | 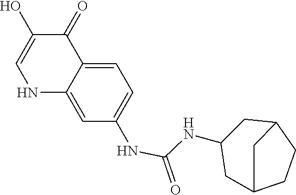 [SMILES: O=C(Nc1ccc2c(=O)c(O)c[nH]c2c1)NC1CC2CCC(C2)C1] |
| 137 | 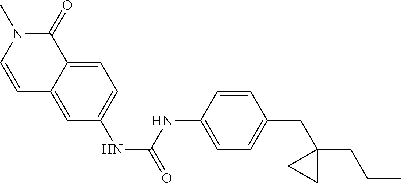 [SMILES: CCCC1(Cc2ccc(NC(=O)Nc3ccc4c(=O)n(C)ccc4c3)cc2)CC1] |
| 138 | 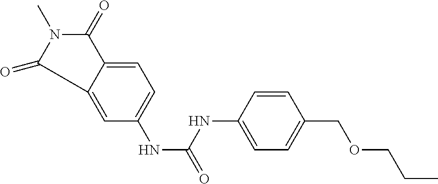 [SMILES: CCCOCc1ccc(NC(=O)Nc2ccc3c(c2)C(=O)N(C)C3=O)cc1] |
| 139 | 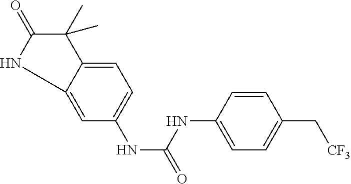 [SMILES: CC1(C)C(=O)Nc2cc(NC(=O)Nc3ccc(CC(F)(F)F)cc3)ccc21] |
| 140 | 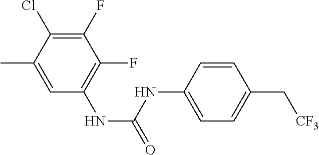 [SMILES: Cc1cc(NC(=O)Nc2ccc(CC(F)(F)F)cc2)c(F)c(F)c1Cl] |
| 141 | 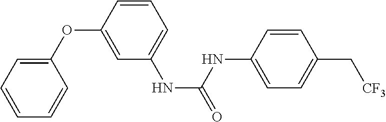 [SMILES: O=C(Nc1ccc(CC(F)(F)F)cc1)Nc1cccc(Oc2ccccc2)c1] |
| 142 | 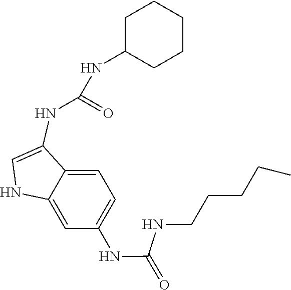 [SMILES: CCCCCNC(=O)Nc1ccc2c(NC(=O)NC3CCCCC3)c[nH]c2c1] |
| 143 | 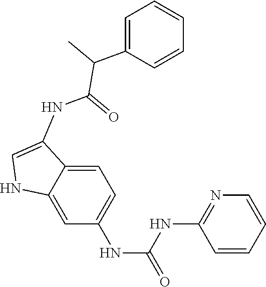 [SMILES: CC(C(=O)Nc1c[nH]c2cc(NC(=O)Nc3ccccn3)ccc12)c1ccccc1] |
| 144 | 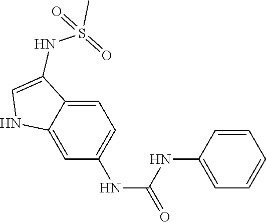 [SMILES: CS(=O)(=O)Nc1c[nH]c2cc(NC(=O)Nc3ccccc3)ccc12] |
| 145 | 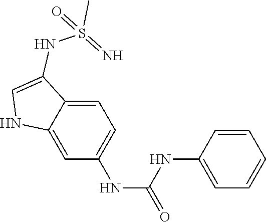 [SMILES: CS(=N)(=O)Nc1c[nH]c2cc(NC(=O)Nc3ccccc3)ccc12] |
| 146 | 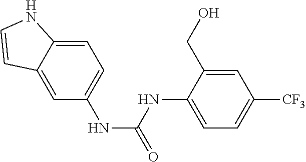 [SMILES: O=C(Nc1ccc2[nH]ccc2c1)Nc1ccc(C(F)(F)F)cc1CO] |
| 147 | 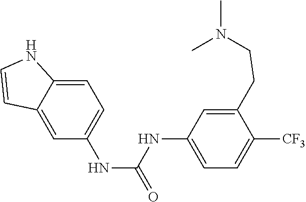 [SMILES: CN(C)CCc1cc(NC(=O)Nc2ccc3[nH]ccc3c2)ccc1C(F)(F)F] |
| 148 | 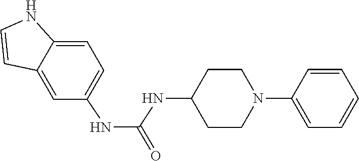 [SMILES: O=C(Nc1ccc2[nH]ccc2c1)NC1CCN(c2ccccc2)CC1] |
| 149 | 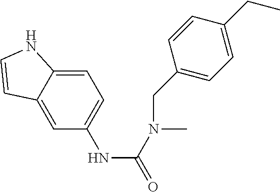 [SMILES: CCc1ccc(CN(C)C(=O)Nc2ccc3[nH]ccc3c2)cc1] |
| 150 | 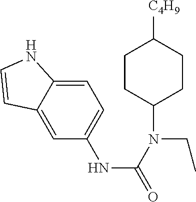 |
| 151 | 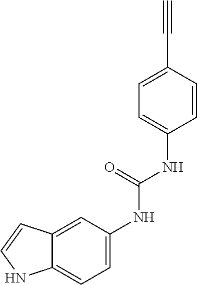 [SMILES: C#Cc1ccc(NC(=O)Nc2ccc3[nH]ccc3c2)cc1] |
| 152 | 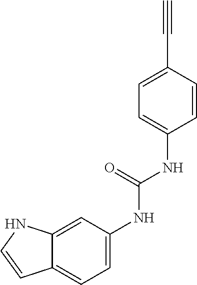 [SMILES: C#Cc1ccc(NC(=O)Nc2ccc3cc[nH]c3c2)cc1] |
| 152a | 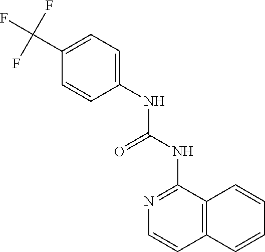 [SMILES: O=C(Nc1ccc(C(F)(F)F)cc1)Nc1nccc2ccccc12] |
| 152b | 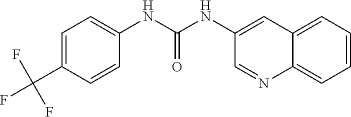 |
| 153 | 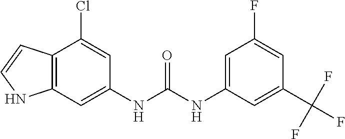 [SMILES: O=C(Nc1cc(F)cc(C(F)(F)F)c1)Nc1cc(Cl)c2cc[nH]c2c1] |
| 154 | 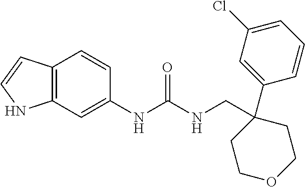 [SMILES: O=C(NCC1(c2cccc(Cl)c2)CCOCC1)Nc1ccc2cc[nH]c2c1] |
| 155 | 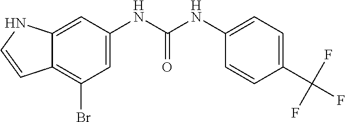 [SMILES: O=C(Nc1ccc(C(F)(F)F)cc1)Nc1cc(Br)c2cc[nH]c2c1] |
| 156 | 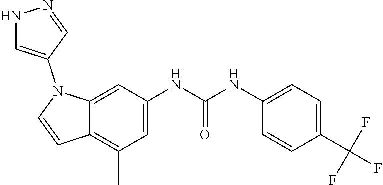 [SMILES: Cc1cc(NC(=O)Nc2ccc(C(F)(F)F)cc2)cc2c1ccn2-c1cn[nH]c1] |
| 157 | 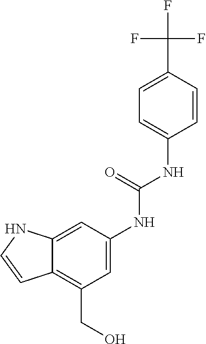 [SMILES: O=C(Nc1ccc(C(F)(F)F)cc1)Nc1cc(CO)c2cc[nH]c2c1] |
| 158 | 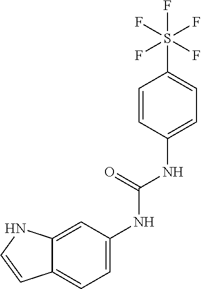 [SMILES: O=C(Nc1ccc(S(F)(F)(F)(F)F)cc1)Nc1ccc2cc[nH]c2c1] |
| 159 | 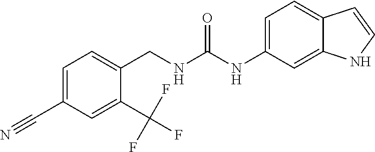 [SMILES: N#Cc1ccc(CNC(=O)Nc2ccc3cc[nH]c3c2)c(C(F)(F)F)c1] |
| 160 | 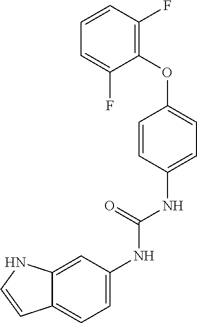 [SMILES: O=C(Nc1ccc(Oc2c(F)cccc2F)cc1)Nc1ccc2cc[nH]c2c1] |
| 161 | 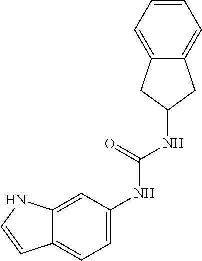 [SMILES: O=C(Nc1ccc2cc[nH]c2c1)NC1Cc2ccccc2C1] |
| 162 | 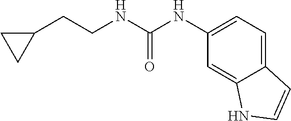 [SMILES: O=C(NCCC1CC1)Nc1ccc2cc[nH]c2c1] |
| 163 | 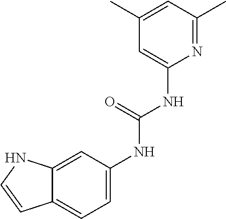 [SMILES: Cc1cc(C)nc(NC(=O)Nc2ccc3cc[nH]c3c2)c1] |
| 164 | 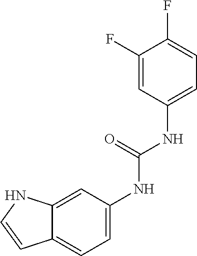 [SMILES: O=C(Nc1ccc(F)c(F)c1)Nc1ccc2cc[nH]c2c1] |
| 165 | 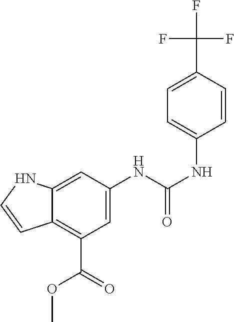 [SMILES: COC(=O)c1cc(NC(=O)Nc2ccc(C(F)(F)F)cc2)cc2[nH]ccc12] |
| 166 | 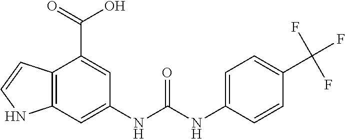 [SMILES: O=C(Nc1ccc(C(F)(F)F)cc1)Nc1cc(C(=O)O)c2cc[nH]c2c1] |
| 167 | 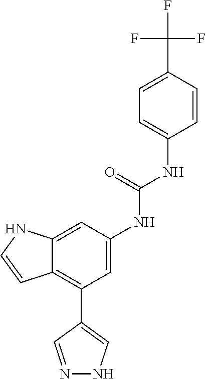 [SMILES: O=C(Nc1ccc(C(F)(F)F)cc1)Nc1cc(-c2cn[nH]c2)c2cc[nH]c2c1] |
| 168 | 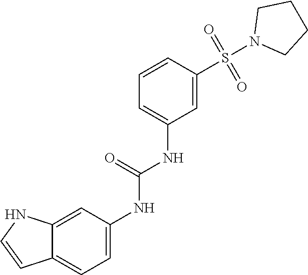 [SMILES: O=C(Nc1cccc(S(=O)(=O)N2CCCC2)c1)Nc1ccc2cc[nH]c2c1] |
| 169 | 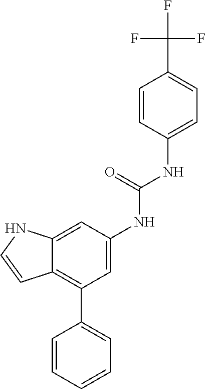 [SMILES: O=C(Nc1ccc(C(F)(F)F)cc1)Nc1cc(-c2ccccc2)c2cc[nH]c2c1] |
| 170 | 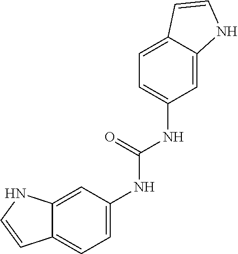 [SMILES: O=C(Nc1ccc2cc[nH]c2c1)Nc1ccc2cc[nH]c2c1] |
| 171 | 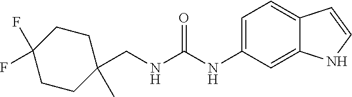 [SMILES: CC1(CNC(=O)Nc2ccc3cc[nH]c3c2)CCC(F)(F)CC1] |
| 172 | 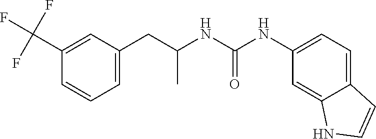 [SMILES: CC(Cc1cccc(C(F)(F)F)c1)NC(=O)Nc1ccc2cc[nH]c2c1] |
| 173 | 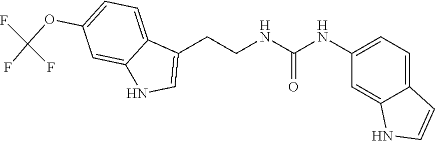 [SMILES: O=C(NCCc1c[nH]c2cc(OC(F)(F)F)ccc12)Nc1ccc2cc[nH]c2c1] |
| 174 | 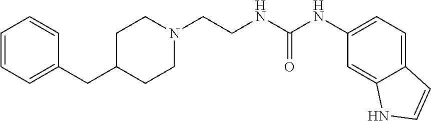 [SMILES: O=C(NCCN1CCC(Cc2ccccc2)CC1)Nc1ccc2cc[nH]c2c1] |
| 175 | 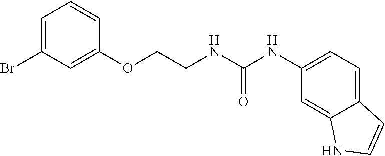 [SMILES: O=C(NCCOc1cccc(Br)c1)Nc1ccc2cc[nH]c2c1] |
| 176 | 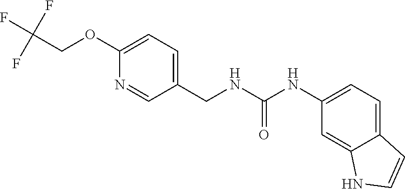 [SMILES: O=C(NCc1ccc(OCC(F)(F)F)nc1)Nc1ccc2cc[nH]c2c1] |
| 177 | 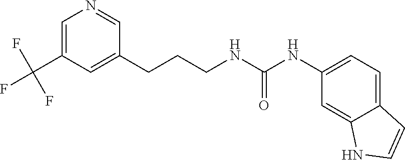 [SMILES: O=C(NCCCc1cncc(C(F)(F)F)c1)Nc1ccc2cc[nH]c2c1] |
| 178 | 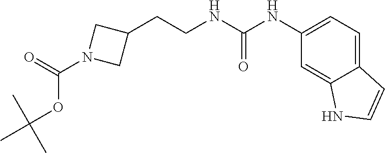 [SMILES: CC(C)(C)OC(=O)N1CC(CCNC(=O)Nc2ccc3cc[nH]c3c2)C1] |
| 179 | 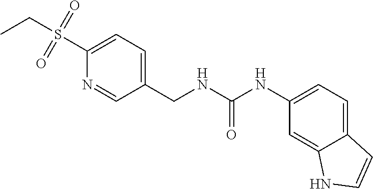 [SMILES: CCS(=O)(=O)c1ccc(CNC(=O)Nc2ccc3cc[nH]c3c2)cn1] |
| 180 | 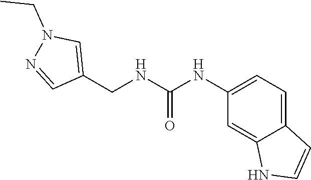 [SMILES: CCn1cc(CNC(=O)Nc2ccc3cc[nH]c3c2)cn1] |
| 181 | 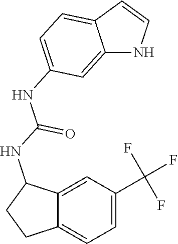 [SMILES: O=C(Nc1ccc2cc[nH]c2c1)NC1CCc2ccc(C(F)(F)F)cc21] |
| 182 | 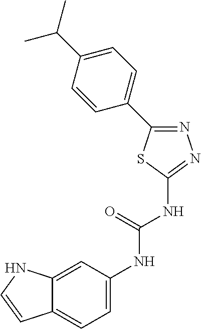 [SMILES: CC(C)c1ccc(-c2nnc(NC(=O)Nc3ccc4cc[nH]c4c3)s2)cc1] |
| 183 | 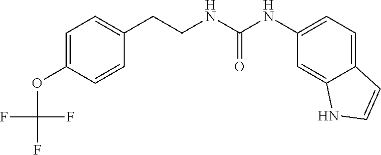 [SMILES: O=C(NCCc1ccc(OC(F)(F)F)cc1)Nc1ccc2cc[nH]c2c1] |
| 184 | 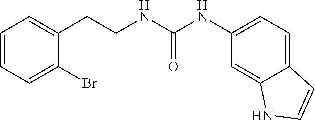 [SMILES: O=C(NCCc1ccccc1Br)Nc1ccc2cc[nH]c2c1] |
| 185 | 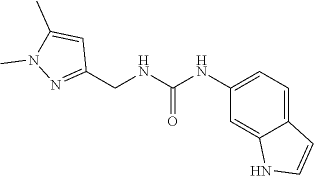 [SMILES: Cc1cc(CNC(=O)Nc2ccc3cc[nH]c3c2)nn1C] |
| 186 | 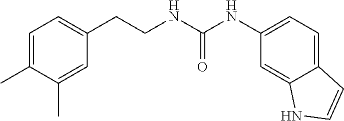 [SMILES: Cc1ccc(CCNC(=O)Nc2ccc3cc[nH]c3c2)cc1C] |
| 187 | 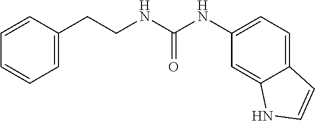 [SMILES: O=C(NCCc1ccccc1)Nc1ccc2cc[nH]c2c1] |
| 188 | 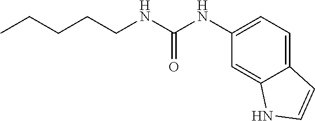 [SMILES: CCCCCNC(=O)Nc1ccc2cc[nH]c2c1] |
| 189 | 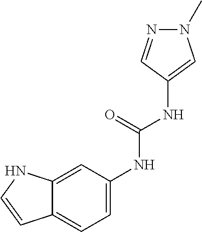 [SMILES: Cn1cc(NC(=O)Nc2ccc3cc[nH]c3c2)cn1] |
| 190 | 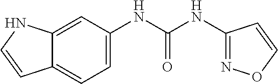 [SMILES: O=C(Nc1ccc2cc[nH]c2c1)Nc1ccon1] |
| 191 | 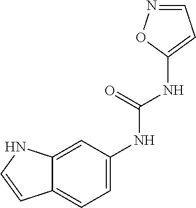 [SMILES: O=C(Nc1ccc2cc[nH]c2c1)Nc1ccno1] |
| 192 | 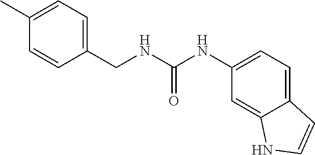 [SMILES: Cc1ccc(CNC(=O)Nc2ccc3cc[nH]c3c2)cc1] |
| 193 | 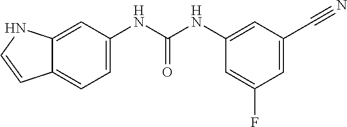 [SMILES: N#Cc1cc(F)cc(NC(=O)Nc2ccc3cc[nH]c3c2)c1] |
| 194 | 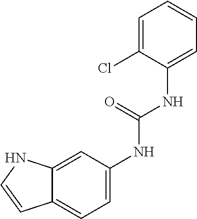 [SMILES: O=C(Nc1ccc2cc[nH]c2c1)Nc1ccccc1Cl] |
| 195 | 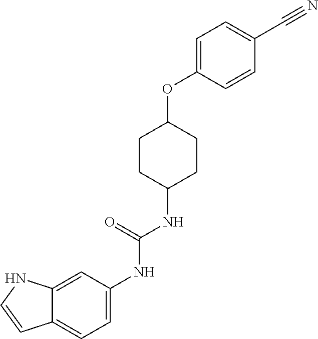 [SMILES: N#Cc1ccc(OC2CCC(NC(=O)Nc3ccc4cc[nH]c4c3)CC2)cc1] |
| 196 | 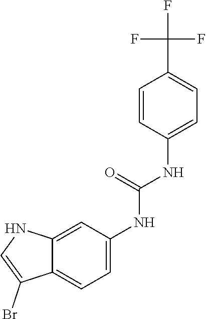 [SMILES: O=C(Nc1ccc(C(F)(F)F)cc1)Nc1ccc2c(Br)c[nH]c2c1] |
| 197 | 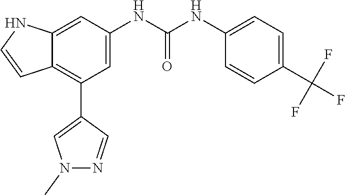 [SMILES: Cn1cc(-c2cc(NC(=O)Nc3ccc(C(F)(F)F)cc3)cc3[nH]ccc23)cn1] |
| 198 | 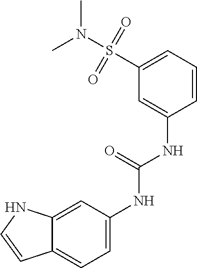 [SMILES: CN(C)S(=O)(=O)c1cccc(NC(=O)Nc2ccc3cc[nH]c3c2)c1] |
| 199 | 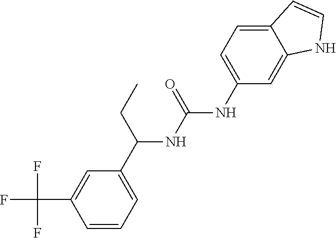 [SMILES: CCC(NC(=O)Nc1ccc2cc[nH]c2c1)c1cccc(C(F)(F)F)c1] |
| 200 | 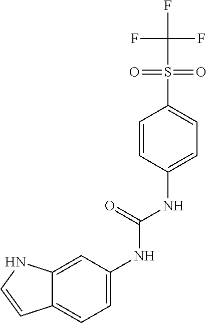 [SMILES: O=C(Nc1ccc(S(=O)(=O)C(F)(F)F)cc1)Nc1ccc2cc[nH]c2c1] |
| 201 | 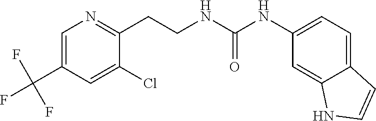 [SMILES: O=C(NCCc1ncc(C(F)(F)F)cc1Cl)Nc1ccc2cc[nH]c2c1] |
| 202 | 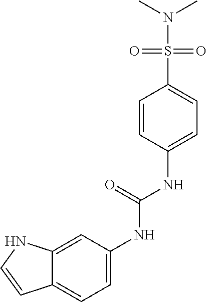 [SMILES: CN(C)S(=O)(=O)c1ccc(NC(=O)Nc2ccc3cc[nH]c3c2)cc1] |
| 203 | 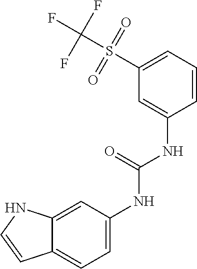 [SMILES: O=C(Nc1cccc(S(=O)(=O)C(F)(F)F)c1)Nc1ccc2cc[nH]c2c1] |
| 204 | 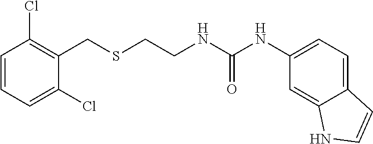 [SMILES: O=C(NCCSCc1c(Cl)cccc1Cl)Nc1ccc2cc[nH]c2c1] |
| 205 | 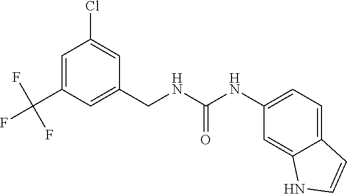 [SMILES: O=C(NCc1cc(Cl)cc(C(F)(F)F)c1)Nc1ccc2cc[nH]c2c1] |
| 206 | 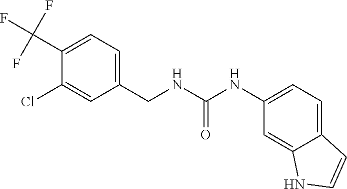 [SMILES: O=C(NCc1ccc(C(F)(F)F)c(Cl)c1)Nc1ccc2cc[nH]c2c1] |
| 207 | 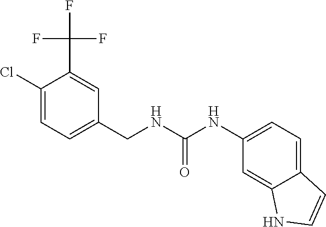 [SMILES: O=C(NCc1ccc(Cl)c(C(F)(F)F)c1)Nc1ccc2cc[nH]c2c1] |
| 208 | 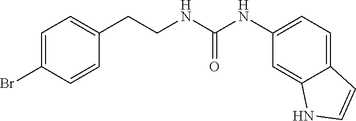 [SMILES: O=C(NCCc1ccc(Br)cc1)Nc1ccc2cc[nH]c2c1] |
| 209 | 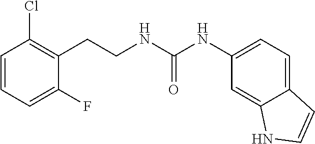 [SMILES: O=C(NCCc1c(F)cccc1Cl)Nc1ccc2cc[nH]c2c1] |
| 210 | 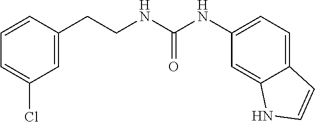 [SMILES: O=C(NCCc1cccc(Cl)c1)Nc1ccc2cc[nH]c2c1] |
| 211 | 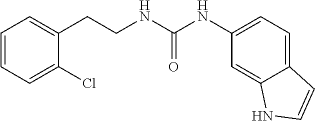 [SMILES: O=C(NCCc1ccccc1Cl)Nc1ccc2cc[nH]c2c1] |
| 212 | 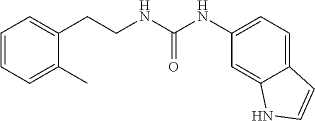 [SMILES: Cc1ccccc1CCNC(=O)Nc1ccc2cc[nH]c2c1] |
| 213 | 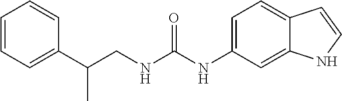 [SMILES: CC(CNC(=O)Nc1ccc2cc[nH]c2c1)c1ccccc1] |
| 214 | 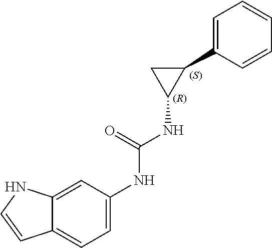 |
| 215 | 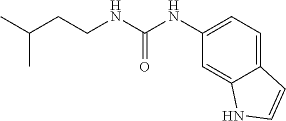 [SMILES: CC(C)CCNC(=O)Nc1ccc2cc[nH]c2c1] |
| 216 | 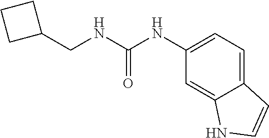 [SMILES: O=C(NCC1CCC1)Nc1ccc2cc[nH]c2c1] |
| 217 | 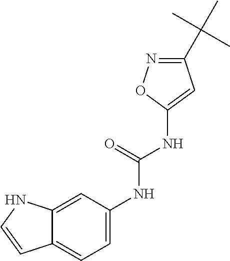 [SMILES: CC(C)(C)c1cc(NC(=O)Nc2ccc3cc[nH]c3c2)on1] |
| 218 | 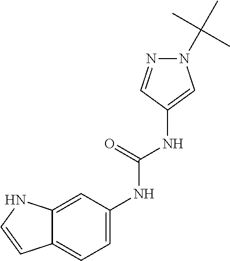 [SMILES: CC(C)(C)n1cc(NC(=O)Nc2ccc3cc[nH]c3c2)cn1] |
| 219 | 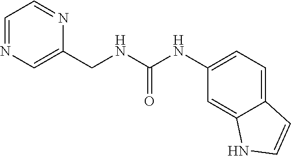 [SMILES: O=C(NCc1cnccn1)Nc1ccc2cc[nH]c2c1] |
| 220 | 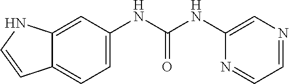 [SMILES: O=C(Nc1ccc2cc[nH]c2c1)Nc1cnccn1] |
| 221 | 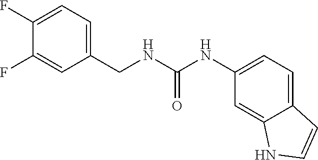 [SMILES: O=C(NCc1ccc(F)c(F)c1)Nc1ccc2cc[nH]c2c1] |
| 222 | 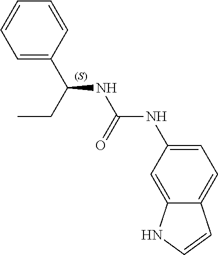 |
| 223 | 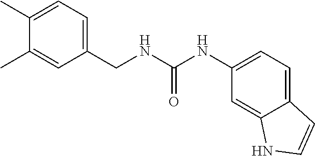 [SMILES: Cc1ccc(CNC(=O)Nc2ccc3cc[nH]c3c2)cc1C] |
| 224 | 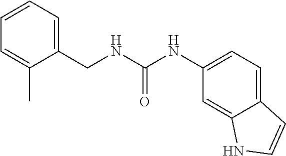 [SMILES: Cc1ccccc1CNC(=O)Nc1ccc2cc[nH]c2c1] |
| 225 | 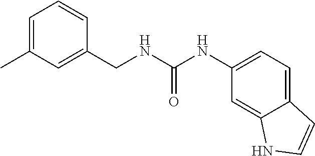 [SMILES: Cc1cccc(CNC(=O)Nc2ccc3cc[nH]c3c2)c1] |
| 226 | 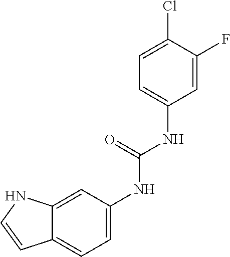 [SMILES: O=C(Nc1ccc(Cl)c(F)c1)Nc1ccc2cc[nH]c2c1] |
| 227 | 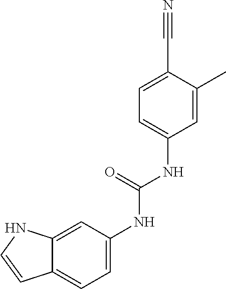 [SMILES: Cc1cc(NC(=O)Nc2ccc3cc[nH]c3c2)ccc1C#N] |
| 228 | 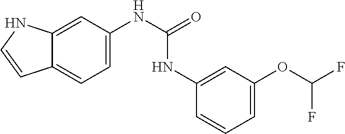 [SMILES: O=C(Nc1cccc(OC(F)F)c1)Nc1ccc2cc[nH]c2c1] |
| 229 | 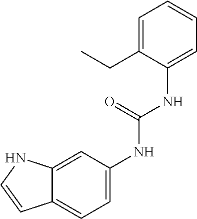 [SMILES: CCc1ccccc1NC(=O)Nc1ccc2cc[nH]c2c1] |
| 230 | 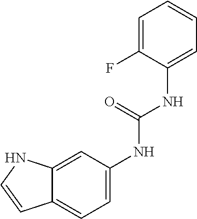 [SMILES: O=C(Nc1ccc2cc[nH]c2c1)Nc1ccccc1F] |
| 231 | 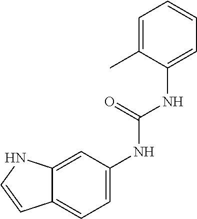 [SMILES: Cc1ccccc1NC(=O)Nc1ccc2cc[nH]c2c1] |
| 233 | 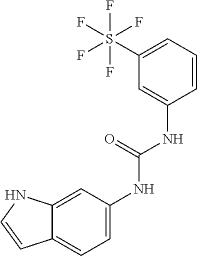 [SMILES: O=C(Nc1cccc(S(F)(F)(F)(F)F)c1)Nc1ccc2cc[nH]c2c1] |
| 234 | 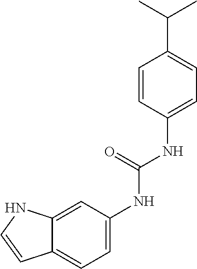 [SMILES: CC(C)c1ccc(NC(=O)Nc2ccc3cc[nH]c3c2)cc1] |
| 235 | 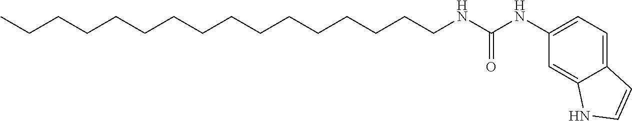 [SMILES: CCCCCCCCCCCCCCCCNC(=O)Nc1ccc2cc[nH]c2c1] |
| 237 | 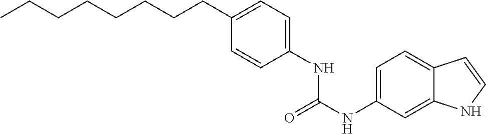 [SMILES: CCCCCCCCc1ccc(NC(=O)Nc2ccc3cc[nH]c3c2)cc1] |
| 236 | 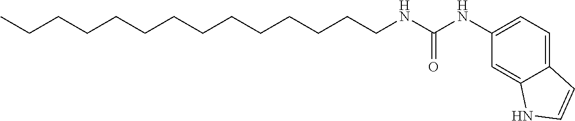 [SMILES: CCCCCCCCCCCCCCNC(=O)Nc1ccc2cc[nH]c2c1] |
| 240 | 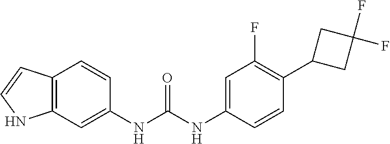 [SMILES: O=C(Nc1ccc(C2CC(F)(F)C2)c(F)c1)Nc1ccc2cc[nH]c2c1] |
| 242 | 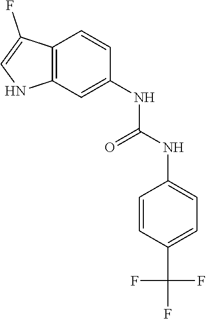 [SMILES: O=C(Nc1ccc(C(F)(F)F)cc1)Nc1ccc2c(F)c[nH]c2c1] |
| 243 | 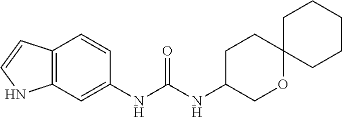 [SMILES: O=C(Nc1ccc2cc[nH]c2c1)NC1CCC2(CCCCC2)OC1] |
| 244 | 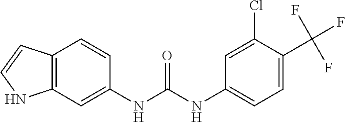 [SMILES: O=C(Nc1ccc(C(F)(F)F)c(Cl)c1)Nc1ccc2cc[nH]c2c1] |
| 245 | 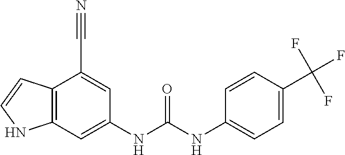 [SMILES: N#Cc1cc(NC(=O)Nc2ccc(C(F)(F)F)cc2)cc2[nH]ccc12] |
| 246 | 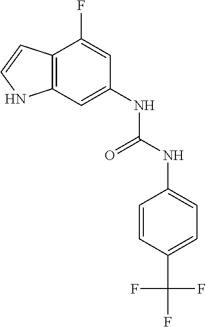 [SMILES: O=C(Nc1ccc(C(F)(F)F)cc1)Nc1cc(F)c2cc[nH]c2c1] |
| 247 | 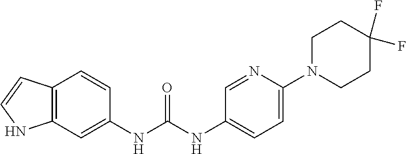 [SMILES: O=C(Nc1ccc(N2CCC(F)(F)CC2)nc1)Nc1ccc2cc[nH]c2c1] |
| 248 | 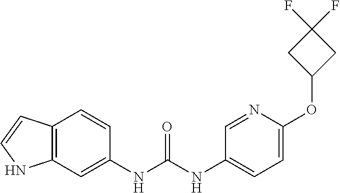 [SMILES: O=C(Nc1ccc(OC2CC(F)(F)C2)nc1)Nc1ccc2cc[nH]c2c1] |
| 249 | 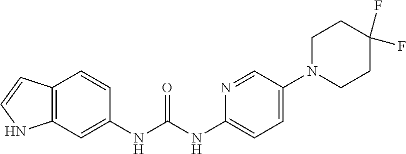 [SMILES: O=C(Nc1ccc2cc[nH]c2c1)Nc1ccc(N2CCC(F)(F)CC2)cn1] |
| 250 | 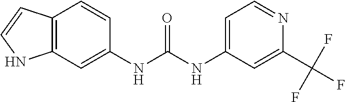 [SMILES: O=C(Nc1ccnc(C(F)(F)F)c1)Nc1ccc2cc[nH]c2c1] |
| 251 | 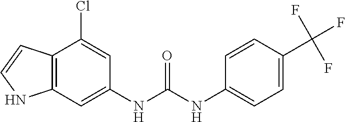 [SMILES: O=C(Nc1ccc(C(F)(F)F)cc1)Nc1cc(Cl)c2cc[nH]c2c1] |
| 252 | 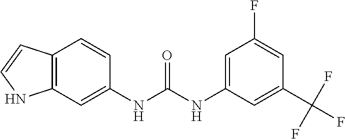 [SMILES: O=C(Nc1cc(F)cc(C(F)(F)F)c1)Nc1ccc2cc[nH]c2c1] |
| 253 | 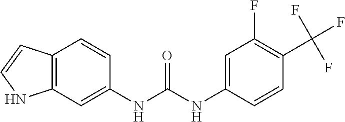 [SMILES: O=C(Nc1ccc(C(F)(F)F)c(F)c1)Nc1ccc2cc[nH]c2c1] |
| 254 | 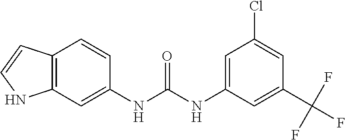 [SMILES: O=C(Nc1cc(Cl)cc(C(F)(F)F)c1)Nc1ccc2cc[nH]c2c1] |
| 255 | 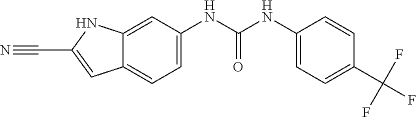 [SMILES: N#Cc1cc2ccc(NC(=O)Nc3ccc(C(F)(F)F)cc3)cc2[nH]1] |
| 256 | 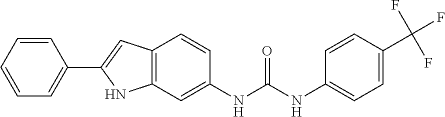 [SMILES: O=C(Nc1ccc(C(F)(F)F)cc1)Nc1ccc2cc(-c3ccccc3)[nH]c2c1] |
| 257 | 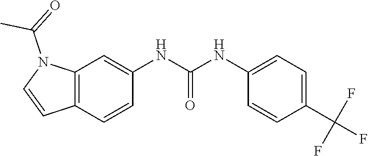 [SMILES: CC(=O)n1ccc2ccc(NC(=O)Nc3ccc(C(F)(F)F)cc3)cc21] |
| 258 | 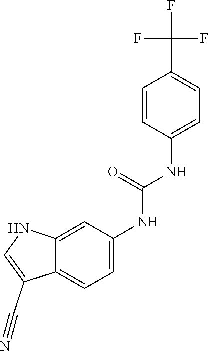 [SMILES: N#Cc1c[nH]c2cc(NC(=O)Nc3ccc(C(F)(F)F)cc3)ccc12] |
| 259 | 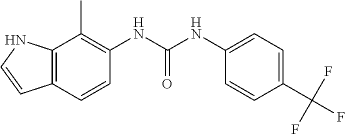 [SMILES: Cc1c(NC(=O)Nc2ccc(C(F)(F)F)cc2)ccc2cc[nH]c12] |
| 260 | 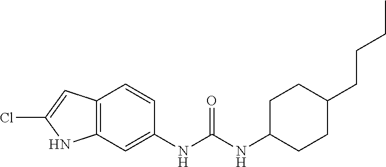 [SMILES: CCCCC1CCC(NC(=O)Nc2ccc3cc(Cl)[nH]c3c2)CC1] |
| 261 | 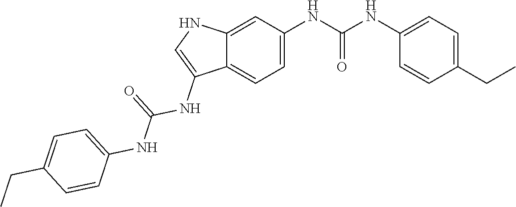 [SMILES: CCc1ccc(NC(=O)Nc2ccc3c(NC(=O)Nc4ccc(CC)cc4)c[nH]c3c2)cc1] |
| 262 | 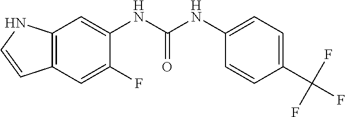 [SMILES: O=C(Nc1ccc(C(F)(F)F)cc1)Nc1cc2[nH]ccc2cc1F] |
| 263 | 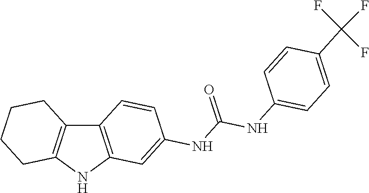 [SMILES: O=C(Nc1ccc(C(F)(F)F)cc1)Nc1ccc2c3c([nH]c2c1)CCCC3] |
| 264 | 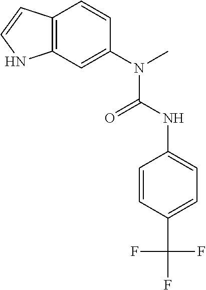 [SMILES: CN(C(=O)Nc1ccc(C(F)(F)F)cc1)c1ccc2cc[nH]c2c1] |
| 265 | 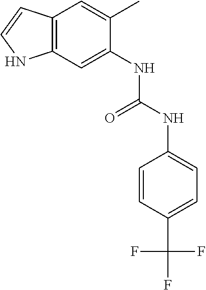 [SMILES: Cc1cc2cc[nH]c2cc1NC(=O)Nc1ccc(C(F)(F)F)cc1] |
| 266 | 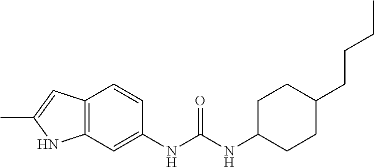 [SMILES: CCCCC1CCC(NC(=O)Nc2ccc3cc(C)[nH]c3c2)CC1] |
| 267 | 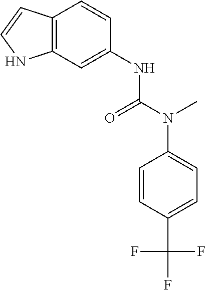 [SMILES: CN(C(=O)Nc1ccc2cc[nH]c2c1)c1ccc(C(F)(F)F)cc1] |
| 268 | 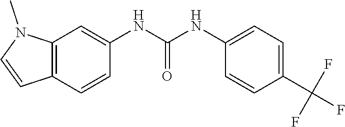 [SMILES: Cn1ccc2ccc(NC(=O)Nc3ccc(C(F)(F)F)cc3)cc21] |
| 269 | 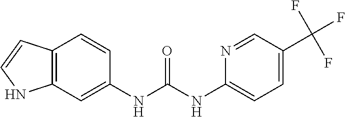 [SMILES: O=C(Nc1ccc2cc[nH]c2c1)Nc1ccc(C(F)(F)F)cn1] |
| 270 | 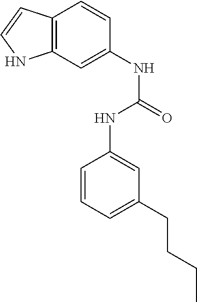 [SMILES: CCCCc1cccc(NC(=O)Nc2ccc3cc[nH]c3c2)c1] |
| 271 | 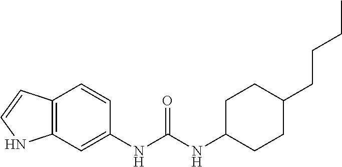 [SMILES: CCCCC1CCC(NC(=O)Nc2ccc3cc[nH]c3c2)CC1] |
| 272 | 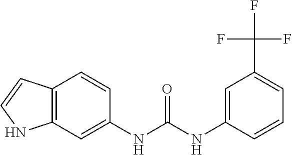 [SMILES: O=C(Nc1cccc(C(F)(F)F)c1)Nc1ccc2cc[nH]c2c1] |
| 273 | 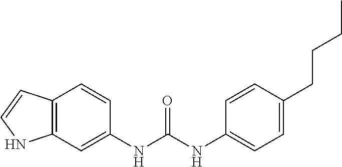 [SMILES: CCCCc1ccc(NC(=O)Nc2ccc3cc[nH]c3c2)cc1] |
| 274 | 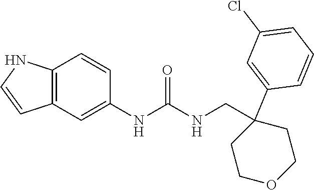 [SMILES: O=C(NCC1(c2cccc(Cl)c2)CCOCC1)Nc1ccc2[nH]ccc2c1] |
| 275 | 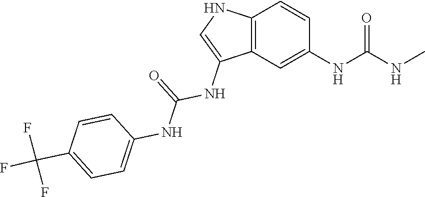 [SMILES: CNC(=O)Nc1ccc2[nH]cc(NC(=O)Nc3ccc(C(F)(F)F)cc3)c2c1] |
| 276 | 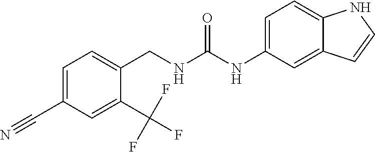 [SMILES: N#Cc1ccc(CNC(=O)Nc2ccc3[nH]ccc3c2)c(C(F)(F)F)c1] |
| 277 | 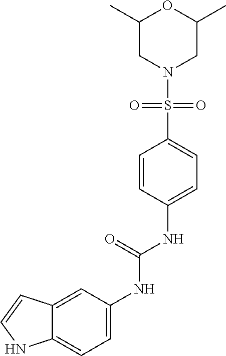 [SMILES: CC1CN(S(=O)(=O)c2ccc(NC(=O)Nc3ccc4[nH]ccc4c3)cc2)CC(C)O1] |
| 278 | 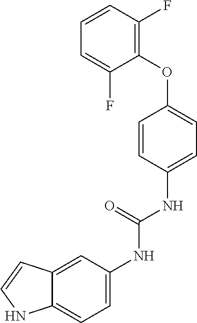 [SMILES: O=C(Nc1ccc(Oc2c(F)cccc2F)cc1)Nc1ccc2[nH]ccc2c1] |
| 279 | 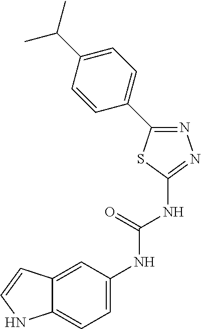 [SMILES: CC(C)c1ccc(-c2nnc(NC(=O)Nc3ccc4[nH]ccc4c3)s2)cc1] |
| 280 | 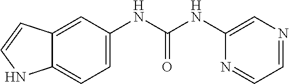 [SMILES: O=C(Nc1ccc2[nH]ccc2c1)Nc1cnccn1] |
| 281 | 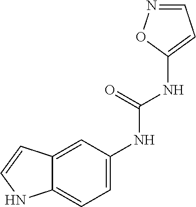 [SMILES: O=C(Nc1ccc2[nH]ccc2c1)Nc1ccno1] |
| 282 | 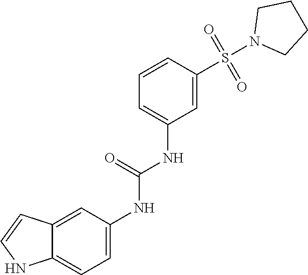 [SMILES: O=C(Nc1cccc(S(=O)(=O)N2CCCC2)c1)Nc1ccc2[nH]ccc2c1] |
| 283 | 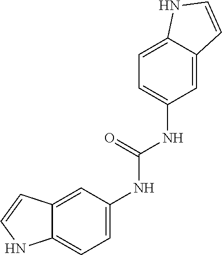 [SMILES: O=C(Nc1ccc2[nH]ccc2c1)Nc1ccc2[nH]ccc2c1] |
| 284 | 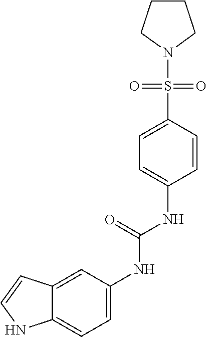 [SMILES: O=C(Nc1ccc(S(=O)(=O)N2CCCC2)cc1)Nc1ccc2[nH]ccc2c1] |
| 285 | 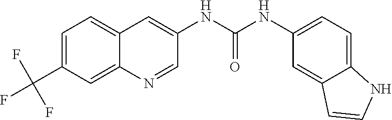 [SMILES: O=C(Nc1cnc2cc(C(F)(F)F)ccc2c1)Nc1ccc2[nH]ccc2c1] |
| 286 | 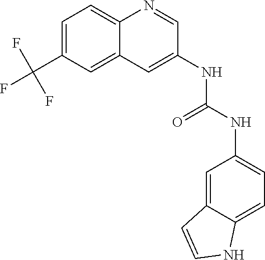 [SMILES: O=C(Nc1cnc2ccc(C(F)(F)F)cc2c1)Nc1ccc2[nH]ccc2c1] |
| 287 | 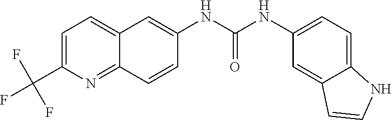 [SMILES: O=C(Nc1ccc2nc(C(F)(F)F)ccc2c1)Nc1ccc2[nH]ccc2c1] |
| 288 | 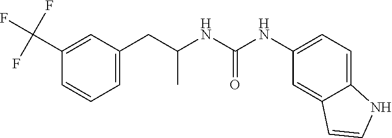 [SMILES: CC(Cc1cccc(C(F)(F)F)c1)NC(=O)Nc1ccc2[nH]ccc2c1] |
| 289 | 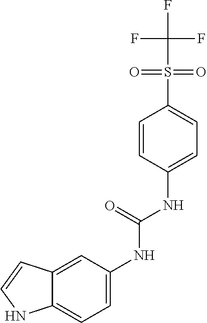 [SMILES: O=C(Nc1ccc(S(=O)(=O)C(F)(F)F)cc1)Nc1ccc2[nH]ccc2c1] |
| 290 | 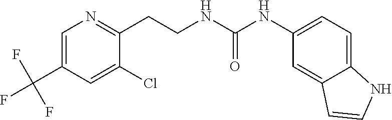 [SMILES: O=C(NCCc1ncc(C(F)(F)F)cc1Cl)Nc1ccc2[nH]ccc2c1] |
| 291 | 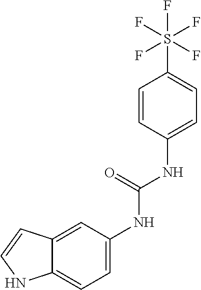 [SMILES: O=C(Nc1ccc(S(F)(F)(F)(F)F)cc1)Nc1ccc2[nH]ccc2c1] |
| 292 | 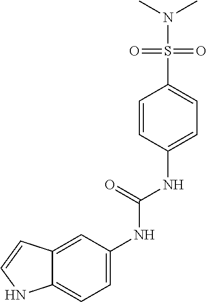 [SMILES: CN(C)S(=O)(=O)c1ccc(NC(=O)Nc2ccc3[nH]ccc3c2)cc1] |
| 293 | 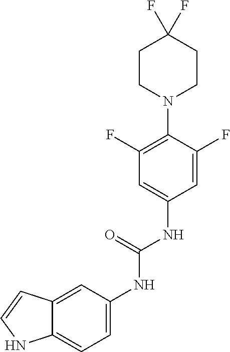 [SMILES: O=C(Nc1cc(F)c(N2CCC(F)(F)CC2)c(F)c1)Nc1ccc2[nH]ccc2c1] |
| 294 | 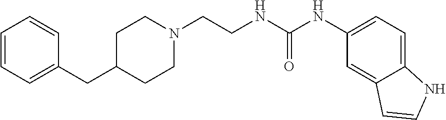 [SMILES: O=C(NCCN1CCC(Cc2ccccc2)CC1)Nc1ccc2[nH]ccc2c1] |
| 295 | 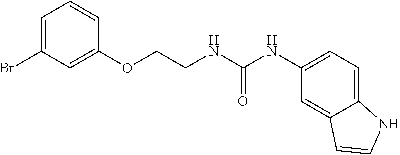 [SMILES: O=C(NCCOc1cccc(Br)c1)Nc1ccc2[nH]ccc2c1] |
| 296 | 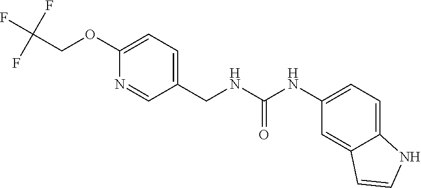 [SMILES: O=C(NCc1ccc(OCC(F)(F)F)nc1)Nc1ccc2[nH]ccc2c1] |
| 297 | 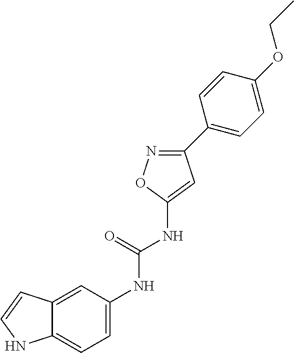 [SMILES: CCOc1ccc(-c2cc(NC(=O)Nc3ccc4[nH]ccc4c3)on2)cc1] |
| 298 | 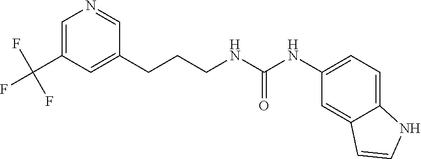 [SMILES: O=C(NCCCc1cncc(C(F)(F)F)c1)Nc1ccc2[nH]ccc2c1] |
| 299 | 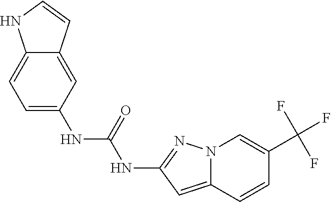 [SMILES: O=C(Nc1ccc2[nH]ccc2c1)Nc1cc2ccc(C(F)(F)F)cn2n1] |
| 300 | 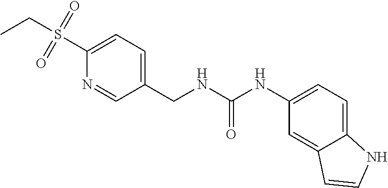 [SMILES: CCS(=O)(=O)c1ccc(CNC(=O)Nc2ccc3[nH]ccc3c2)cn1] |
| 301 | 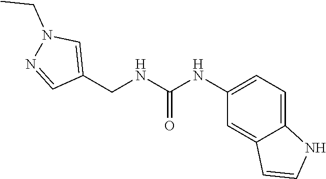 [SMILES: CCn1cc(CNC(=O)Nc2ccc3[nH]ccc3c2)cn1] |
| 302 | 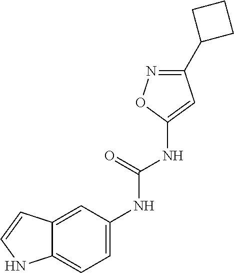 [SMILES: O=C(Nc1ccc2[nH]ccc2c1)Nc1cc(C2CCC2)no1] |
| 303 | 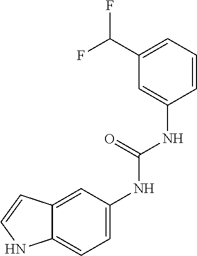 [SMILES: O=C(Nc1cccc(C(F)F)c1)Nc1ccc2[nH]ccc2c1] |
| 304 | 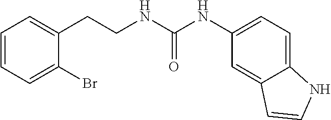 [SMILES: O=C(NCCc1ccccc1Br)Nc1ccc2[nH]ccc2c1] |
| 305 | 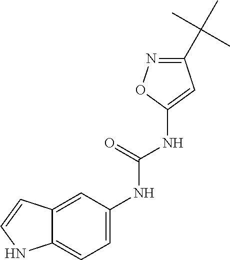 [SMILES: CC(C)(C)c1cc(NC(=O)Nc2ccc3[nH]ccc3c2)on1] |
| 306 | 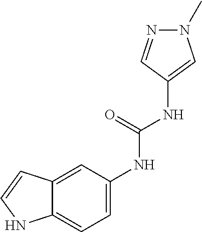 [SMILES: Cn1cc(NC(=O)Nc2ccc3[nH]ccc3c2)cn1] |
| 307 | 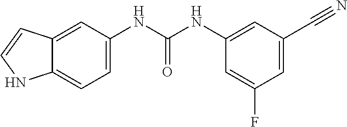 [SMILES: N#Cc1cc(F)cc(NC(=O)Nc2ccc3[nH]ccc3c2)c1] |
| 308 | 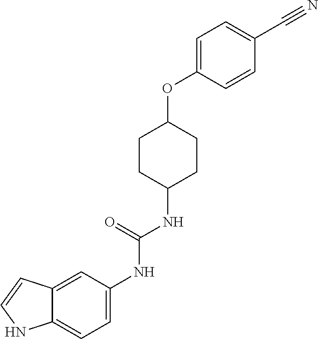 [SMILES: N#Cc1ccc(OC2CCC(NC(=O)Nc3ccc4[nH]ccc4c3)CC2)cc1] |
| 309 | 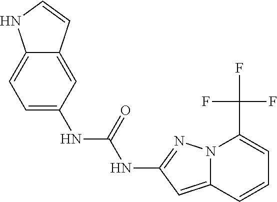 [SMILES: O=C(Nc1ccc2[nH]ccc2c1)Nc1cc2cccc(C(F)(F)F)n2n1] |
| 311 | 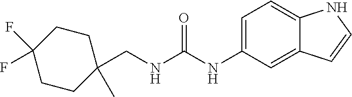 [SMILES: CC1(CNC(=O)Nc2ccc3[nH]ccc3c2)CCC(F)(F)CC1] |
| 312 | 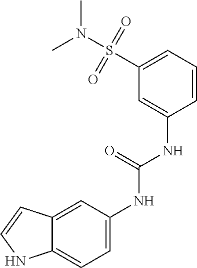 [SMILES: CN(C)S(=O)(=O)c1cccc(NC(=O)Nc2ccc3[nH]ccc3c2)c1] |
| 313 | 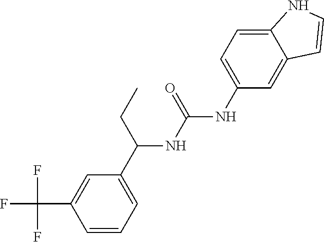 [SMILES: CCC(NC(=O)Nc1ccc2[nH]ccc2c1)c1cccc(C(F)(F)F)c1] |
| 314 | 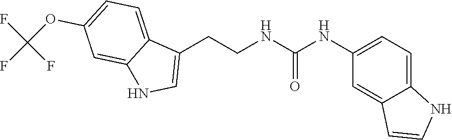 [SMILES: O=C(NCCc1c[nH]c2cc(OC(F)(F)F)ccc12)Nc1ccc2[nH]ccc2c1] |
| 315 | 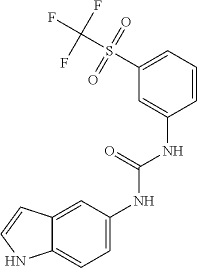 [SMILES: O=C(Nc1cccc(S(=O)(=O)C(F)(F)F)c1)Nc1ccc2[nH]ccc2c1] |
| 316 | 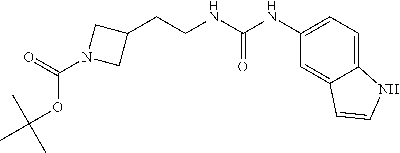 [SMILES: CC(C)(C)OC(=O)N1CC(CCNC(=O)Nc2ccc3[nH]ccc3c2)C1] |
| 317 | 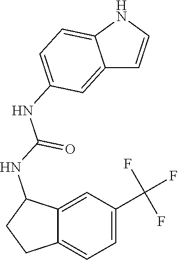 [SMILES: O=C(Nc1ccc2[nH]ccc2c1)NC1CCc2ccc(C(F)(F)F)cc21] |
| 318 | 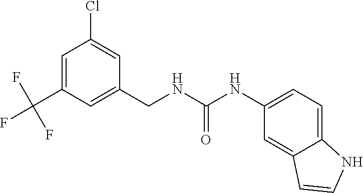 [SMILES: O=C(NCc1cc(Cl)cc(C(F)(F)F)c1)Nc1ccc2[nH]ccc2c1] |
| 319 | 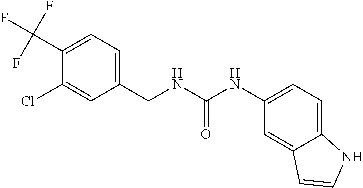 [SMILES: O=C(NCc1ccc(C(F)(F)F)c(Cl)c1)Nc1ccc2[nH]ccc2c1] |
| 320 | 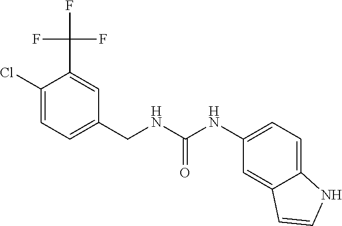 [SMILES: O=C(NCc1ccc(Cl)c(C(F)(F)F)c1)Nc1ccc2[nH]ccc2c1] |
| 321 | 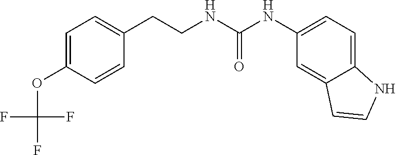 [SMILES: O=C(NCCc1ccc(OC(F)(F)F)cc1)Nc1ccc2[nH]ccc2c1] |
| 322 | 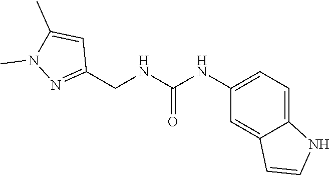 [SMILES: Cc1cc(CNC(=O)Nc2ccc3[nH]ccc3c2)nn1C] |
| 323 | 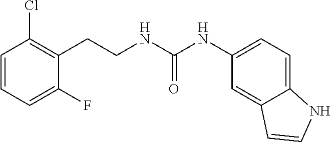 [SMILES: O=C(NCCc1c(F)cccc1Cl)Nc1ccc2[nH]ccc2c1] |
| 324 | 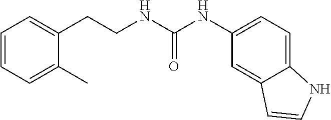 [SMILES: Cc1ccccc1CCNC(=O)Nc1ccc2[nH]ccc2c1] |
| 325 | 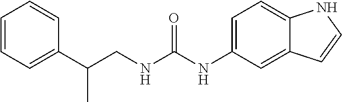 [SMILES: CC(CNC(=O)Nc1ccc2[nH]ccc2c1)c1ccccc1] |
| 326 | 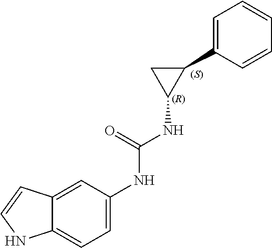 |
| 327 | 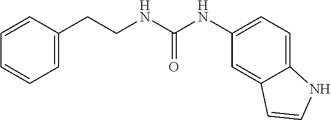 [SMILES: O=C(NCCc1ccccc1)Nc1ccc2[nH]ccc2c1] |
| 328 | 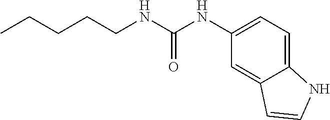 [SMILES: CCCCCNC(=O)Nc1ccc2[nH]ccc2c1] |
| 329 | 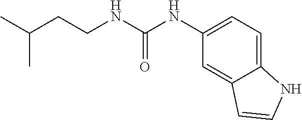 [SMILES: CC(C)CCNC(=O)Nc1ccc2[nH]ccc2c1] |
| 330 | 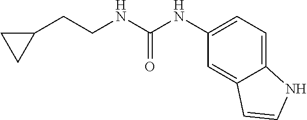 [SMILES: O=C(NCCC1CC1)Nc1ccc2[nH]ccc2c1] |
| 331 | 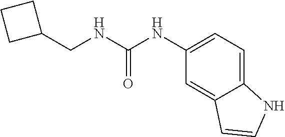 [SMILES: O=C(NCC1CCC1)Nc1ccc2[nH]ccc2c1] |
| 332 | 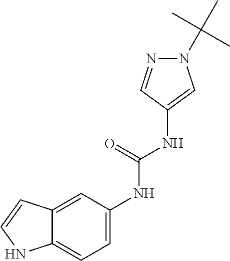 [SMILES: CC(C)(C)n1cc(NC(=O)Nc2ccc3[nH]ccc3c2)cn1] |
| 333 | 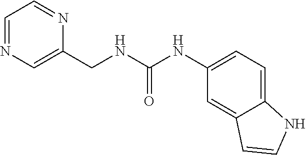 [SMILES: O=C(NCc1cnccn1)Nc1ccc2[nH]ccc2c1] |
| 334 | 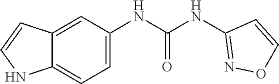 [SMILES: O=C(Nc1ccc2[nH]ccc2c1)Nc1ccon1] |
| 335 | 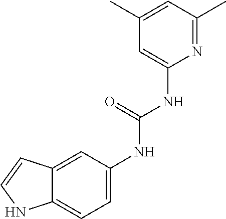 [SMILES: Cc1cc(C)nc(NC(=O)Nc2ccc3[nH]ccc3c2)c1] |
| 336 | 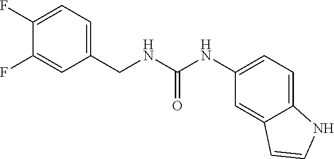 [SMILES: O=C(NCc1ccc(F)c(F)c1)Nc1ccc2[nH]ccc2c1] |
| 337 | 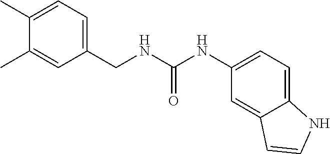 [SMILES: Cc1ccc(CNC(=O)Nc2ccc3[nH]ccc3c2)cc1C] |
| 338 | 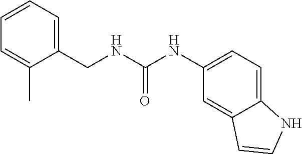 [SMILES: Cc1ccccc1CNC(=O)Nc1ccc2[nH]ccc2c1] |
| 339 | 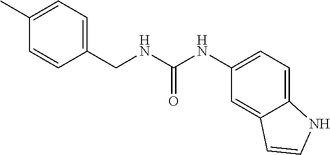 [SMILES: Cc1ccc(CNC(=O)Nc2ccc3[nH]ccc3c2)cc1] |
| 340 | 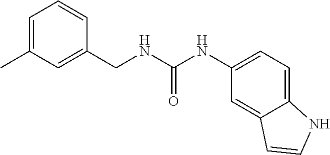 [SMILES: Cc1cccc(CNC(=O)Nc2ccc3[nH]ccc3c2)c1] |
| 341 | 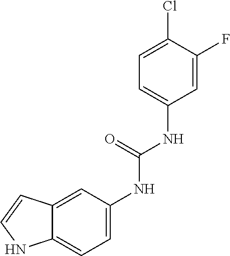 [SMILES: O=C(Nc1ccc(Cl)c(F)c1)Nc1ccc2[nH]ccc2c1] |
| 342 | 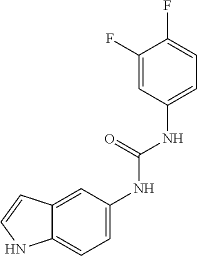 [SMILES: O=C(Nc1ccc(F)c(F)c1)Nc1ccc2[nH]ccc2c1] |
| 343 | 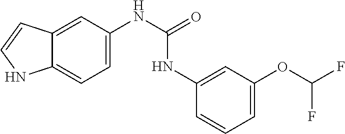 [SMILES: O=C(Nc1cccc(OC(F)F)c1)Nc1ccc2[nH]ccc2c1] |
| 345 | 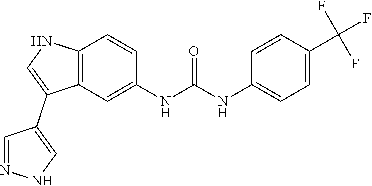 [SMILES: O=C(Nc1ccc(C(F)(F)F)cc1)Nc1ccc2[nH]cc(-c3cn[nH]c3)c2c1] |
| 346 | 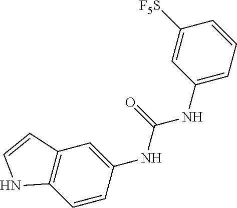 |
| 347 | 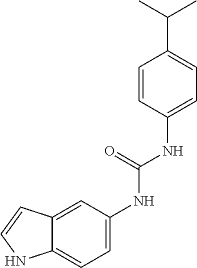 [SMILES: CC(C)c1ccc(NC(=O)Nc2ccc3[nH]ccc3c2)cc1] |
| 348 | 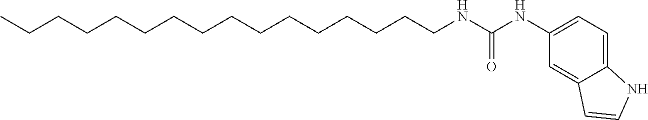 [SMILES: CCCCCCCCCCCCCCCCNC(=O)Nc1ccc2[nH]ccc2c1] |
| 349 | 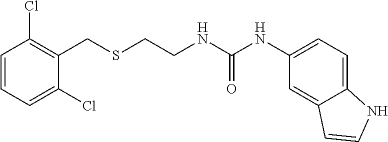 [SMILES: O=C(NCCSCc1c(Cl)cccc1Cl)Nc1ccc2[nH]ccc2c1] |
| 350 | 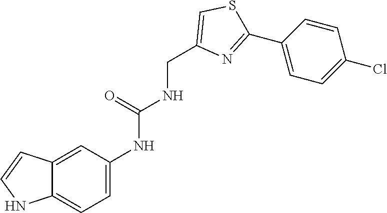 [SMILES: O=C(NCc1csc(-c2ccc(Cl)cc2)n1)Nc1ccc2[nH]ccc2c1] |
| 352 | 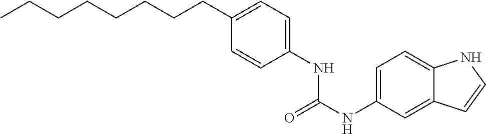 [SMILES: CCCCCCCCc1ccc(NC(=O)Nc2ccc3[nH]ccc3c2)cc1] |
| 351 | 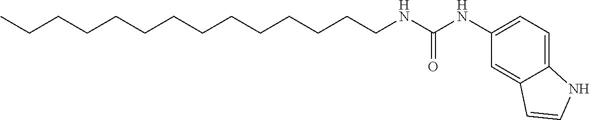 [SMILES: CCCCCCCCCCCCCCNC(=O)Nc1ccc2[nH]ccc2c1] |
| 353 | 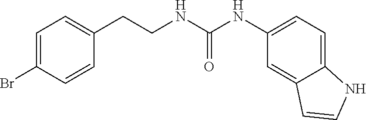 [SMILES: O=C(NCCc1ccc(Br)cc1)Nc1ccc2[nH]ccc2c1] |
| 354 | 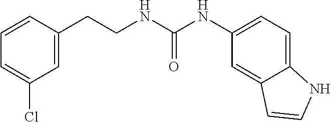 [SMILES: O=C(NCCc1cccc(Cl)c1)Nc1ccc2[nH]ccc2c1] |
| 355 | 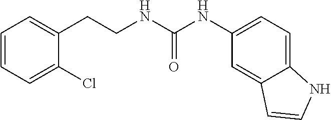 [SMILES: O=C(NCCc1ccccc1Cl)Nc1ccc2[nH]ccc2c1] |
| 356 | 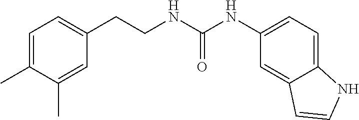 [SMILES: Cc1ccc(CCNC(=O)Nc2ccc3[nH]ccc3c2)cc1C] |
| 357 | 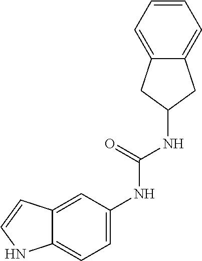 [SMILES: O=C(Nc1ccc2[nH]ccc2c1)NC1Cc2ccccc2C1] |
| 358 | 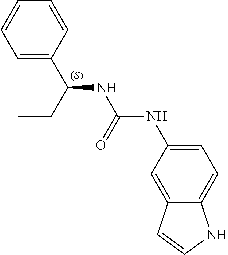 |
| 359 | 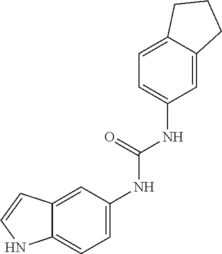 [SMILES: O=C(Nc1ccc2c(c1)CCC2)Nc1ccc2[nH]ccc2c1] |
| 360 | 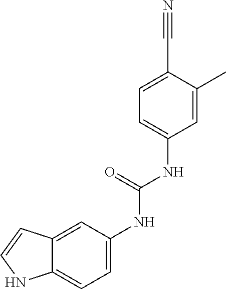 [SMILES: Cc1cc(NC(=O)Nc2ccc3[nH]ccc3c2)ccc1C#N] |
| 361 | 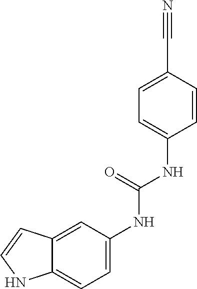 [SMILES: N#Cc1ccc(NC(=O)Nc2ccc3[nH]ccc3c2)cc1] |
| 362 | 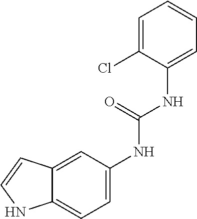 [SMILES: O=C(Nc1ccc2[nH]ccc2c1)Nc1ccccc1Cl] |
| 363 | 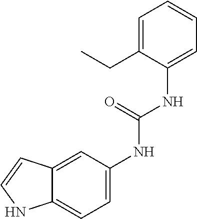 [SMILES: CCc1ccccc1NC(=O)Nc1ccc2[nH]ccc2c1] |
| 364 | 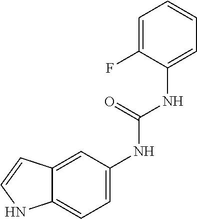 [SMILES: O=C(Nc1ccc2[nH]ccc2c1)Nc1ccccc1F] |
| 365 | 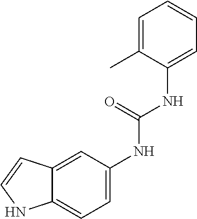 [SMILES: Cc1ccccc1NC(=O)Nc1ccc2[nH]ccc2c1] |
| 366 | 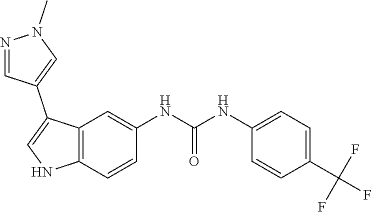 [SMILES: Cn1cc(-c2c[nH]c3ccc(NC(=O)Nc4ccc(C(F)(F)F)cc4)cc23)cn1] |
| 370 | 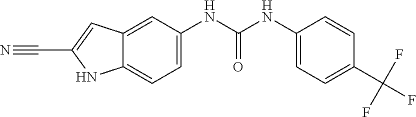 [SMILES: N#Cc1cc2cc(NC(=O)Nc3ccc(C(F)(F)F)cc3)ccc2[nH]1] |
| 371 | 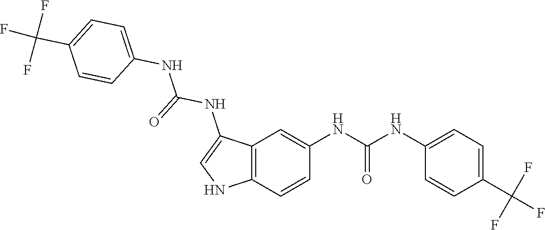 [SMILES: O=C(Nc1ccc(C(F)(F)F)cc1)Nc1ccc2[nH]cc(NC(=O)Nc3ccc(C(F)(F)F)cc3)c2c1] |
| 372 | 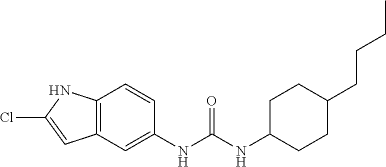 [SMILES: CCCCC1CCC(NC(=O)Nc2ccc3[nH]c(Cl)cc3c2)CC1] |
| 373 | 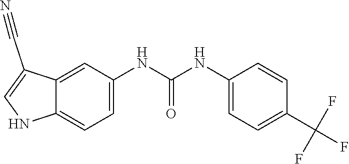 [SMILES: N#Cc1c[nH]c2ccc(NC(=O)Nc3ccc(C(F)(F)F)cc3)cc12] |
| 374 | 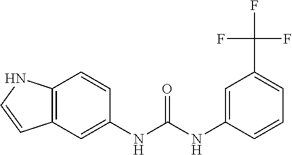 [SMILES: O=C(Nc1cccc(C(F)(F)F)c1)Nc1ccc2[nH]ccc2c1] |
| 375 | 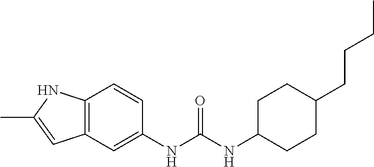 [SMILES: CCCCC1CCC(NC(=O)Nc2ccc3[nH]c(C)cc3c2)CC1] |
| 376 | 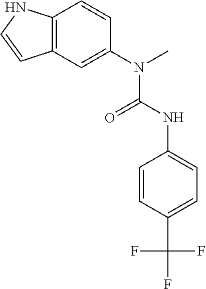 [SMILES: CN(C(=O)Nc1ccc(C(F)(F)F)cc1)c1ccc2[nH]ccc2c1] |
| 377 | 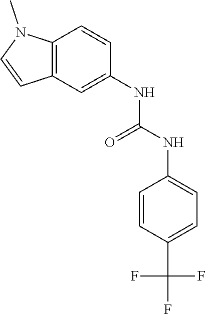 [SMILES: Cn1ccc2cc(NC(=O)Nc3ccc(C(F)(F)F)cc3)ccc21] |
| 378 | 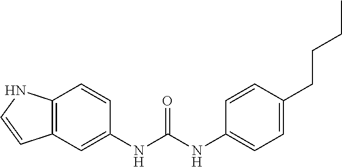 [SMILES: CCCCc1ccc(NC(=O)Nc2ccc3[nH]ccc3c2)cc1] |
| 379 | 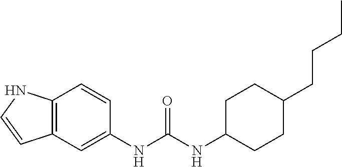 [SMILES: CCCCC1CCC(NC(=O)Nc2ccc3[nH]ccc3c2)CC1] |
| 380 | 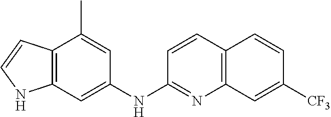 [SMILES: Cc1cc(Nc2ccc3ccc(C(F)(F)F)cc3n2)cc2[nH]ccc12] |
| 381 | 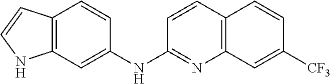 [SMILES: FC(F)(F)c1ccc2ccc(Nc3ccc4cc[nH]c4c3)nc2c1] |
| 382 | 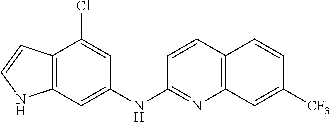 [SMILES: FC(F)(F)c1ccc2ccc(Nc3cc(Cl)c4cc[nH]c4c3)nc2c1] |
| 383 | 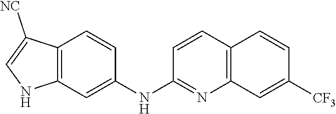 [SMILES: N#Cc1c[nH]c2cc(Nc3ccc4ccc(C(F)(F)F)cc4n3)ccc12] |
| 384 | 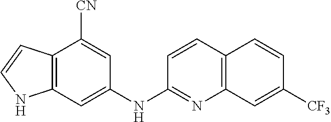 [SMILES: N#Cc1cc(Nc2ccc3ccc(C(F)(F)F)cc3n2)cc2[nH]ccc12] |
| 385 | 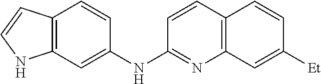 [SMILES: CCc1ccc2ccc(Nc3ccc4cc[nH]c4c3)nc2c1] |
| 386 | 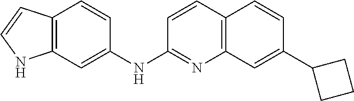 [SMILES: c1cc2ccc(Nc3ccc4ccc(C5CCC5)cc4n3)cc2[nH]1] |
| 387 | 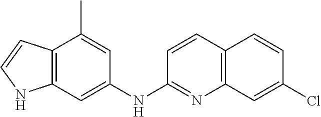 [SMILES: Cc1cc(Nc2ccc3ccc(Cl)cc3n2)cc2[nH]ccc12] |
| 388 | 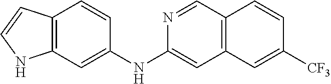 [SMILES: FC(F)(F)c1ccc2cnc(Nc3ccc4cc[nH]c4c3)cc2c1] |
| 389 | 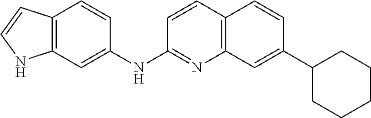 [SMILES: c1cc2ccc(Nc3ccc4ccc(C5CCCCC5)cc4n3)cc2[nH]1] |
| 390 | 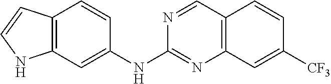 [SMILES: FC(F)(F)c1ccc2cnc(Nc3ccc4cc[nH]c4c3)nc2c1] |
| 391 | 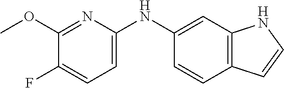 [SMILES: COc1nc(Nc2ccc3cc[nH]c3c2)ccc1F] |
| 392 | 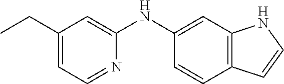 [SMILES: CCc1ccnc(Nc2ccc3cc[nH]c3c2)c1] |
| 393 | 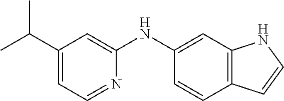 [SMILES: CC(C)c1ccnc(Nc2ccc3cc[nH]c3c2)c1] |
| 394 | 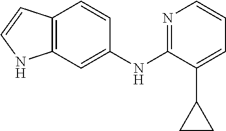 [SMILES: c1cnc(Nc2ccc3cc[nH]c3c2)c(C2CC2)c1] |
| 395 | 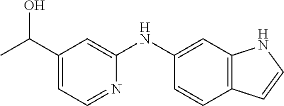 [SMILES: CC(O)c1ccnc(Nc2ccc3cc[nH]c3c2)c1] |
| 396 | 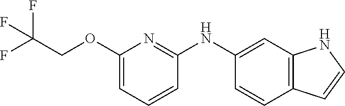 [SMILES: FC(F)(F)COc1cccc(Nc2ccc3cc[nH]c3c2)n1] |
| 397 | 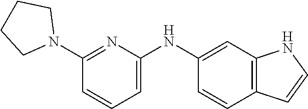 [SMILES: c1cc(Nc2ccc3cc[nH]c3c2)nc(N2CCCC2)c1] |
| 398 | 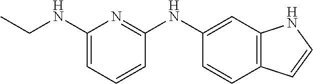 [SMILES: CCNc1cccc(Nc2ccc3cc[nH]c3c2)n1] |
| 399 | 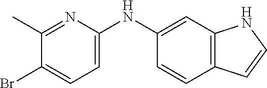 [SMILES: Cc1nc(Nc2ccc3cc[nH]c3c2)ccc1Br] |
| 400 | 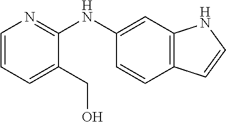 [SMILES: OCc1cccnc1Nc1ccc2cc[nH]c2c1] |
| 401 | 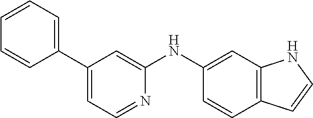 [SMILES: c1ccc(-c2ccnc(Nc3ccc4cc[nH]c4c3)c2)cc1] |
| 402 | 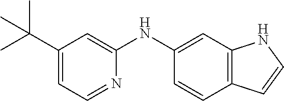 [SMILES: CC(C)(C)c1ccnc(Nc2ccc3cc[nH]c3c2)c1] |
| 403 | 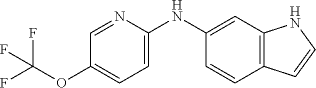 [SMILES: FC(F)(F)Oc1ccc(Nc2ccc3cc[nH]c3c2)nc1] |
| 404 | 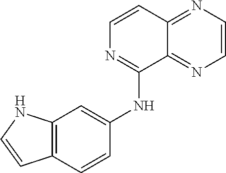 [SMILES: c1cc2nccnc2c(Nc2ccc3cc[nH]c3c2)n1] |
| 405 | 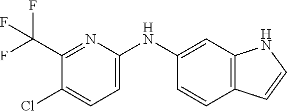 [SMILES: FC(F)(F)c1nc(Nc2ccc3cc[nH]c3c2)ccc1Cl] |
| 406 | 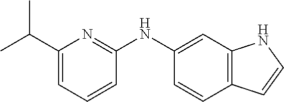 [SMILES: CC(C)c1cccc(Nc2ccc3cc[nH]c3c2)n1] |
| 407 | 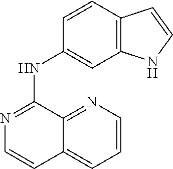 [SMILES: c1cnc2c(Nc3ccc4cc[nH]c4c3)nccc2c1] |
| 408 | 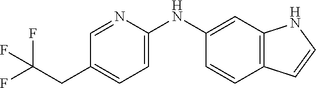 [SMILES: FC(F)(F)Cc1ccc(Nc2ccc3cc[nH]c3c2)nc1] |
| 409 | 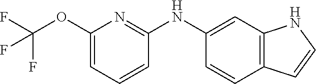 [SMILES: FC(F)(F)Oc1cccc(Nc2ccc3cc[nH]c3c2)n1] |
| 410 | 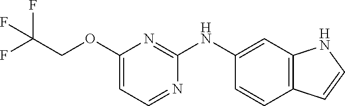 [SMILES: FC(F)(F)COc1ccnc(Nc2ccc3cc[nH]c3c2)n1] |
| 411 | 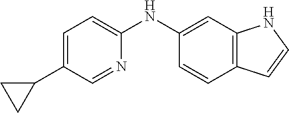 [SMILES: c1cc2ccc(Nc3ccc(C4CC4)cn3)cc2[nH]1] |
| 412 | 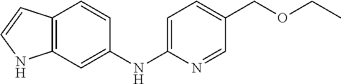 [SMILES: CCOCc1ccc(Nc2ccc3cc[nH]c3c2)nc1] |
| 413 | 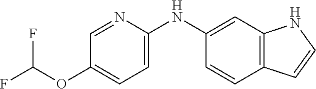 [SMILES: FC(F)Oc1ccc(Nc2ccc3cc[nH]c3c2)nc1] |
| 414 | 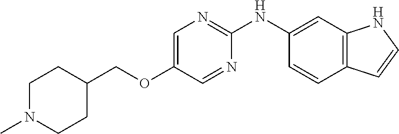 [SMILES: CN1CCC(COc2cnc(Nc3ccc4cc[nH]c4c3)nc2)CC1] |
| 415 | 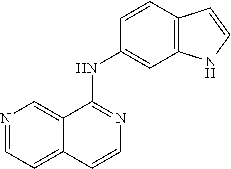 [SMILES: c1cc2ccnc(Nc3ccc4cc[nH]c4c3)c2cn1] |
| 416 | 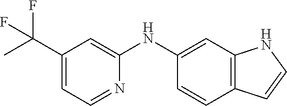 [SMILES: CC(F)(F)c1ccnc(Nc2ccc3cc[nH]c3c2)c1] |
| 417 | 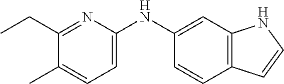 [SMILES: CCc1nc(Nc2ccc3cc[nH]c3c2)ccc1C] |
| 418 | 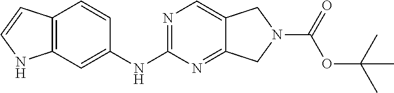 [SMILES: CC(C)(C)OC(=O)N1Cc2cnc(Nc3ccc4cc[nH]c4c3)nc2C1] |
| 419 | 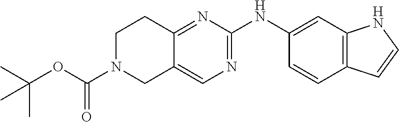 [SMILES: CC(C)(C)OC(=O)N1CCc2nc(Nc3ccc4cc[nH]c4c3)ncc2C1] |
| 420 | 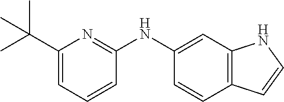 [SMILES: CC(C)(C)c1cccc(Nc2ccc3cc[nH]c3c2)n1] |
| 421 | 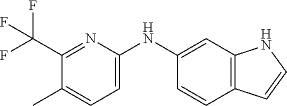 [SMILES: Cc1ccc(Nc2ccc3cc[nH]c3c2)nc1C(F)(F)F] |
| 422 | 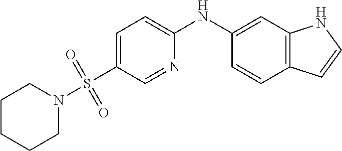 [SMILES: O=S(=O)(c1ccc(Nc2ccc3cc[nH]c3c2)nc1)N1CCCCC1] |
| 423 | 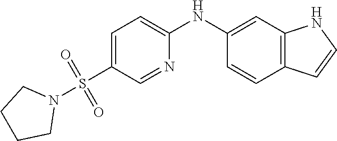 [SMILES: O=S(=O)(c1ccc(Nc2ccc3cc[nH]c3c2)nc1)N1CCCC1] |
| 424 | 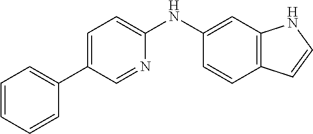 [SMILES: c1ccc(-c2ccc(Nc3ccc4cc[nH]c4c3)nc2)cc1] |
| 425 | 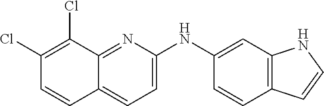 [SMILES: Clc1ccc2ccc(Nc3ccc4cc[nH]c4c3)nc2c1Cl] |
| 426 | 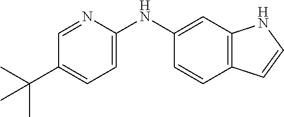 [SMILES: CC(C)(C)c1ccc(Nc2ccc3cc[nH]c3c2)nc1] |
| 427 | 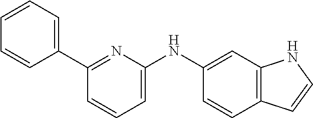 [SMILES: c1ccc(-c2cccc(Nc3ccc4cc[nH]c4c3)n2)cc1] |
| 428 | 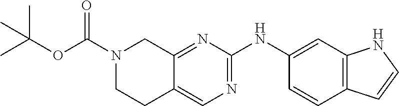 [SMILES: CC(C)(C)OC(=O)N1CCc2cnc(Nc3ccc4cc[nH]c4c3)nc2C1] |
| 429 | 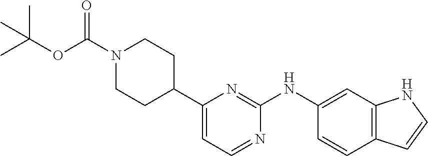 [SMILES: CC(C)(C)OC(=O)N1CCC(c2ccnc(Nc3ccc4cc[nH]c4c3)n2)CC1] |
| 430 | 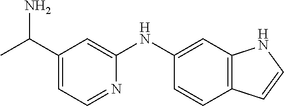 [SMILES: CC(N)c1ccnc(Nc2ccc3cc[nH]c3c2)c1] |
| 431 | 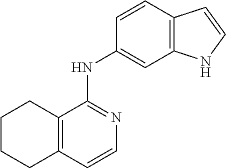 [SMILES: c1cc2c(c(Nc3ccc4cc[nH]c4c3)n1)CCCC2] |
| 432 | 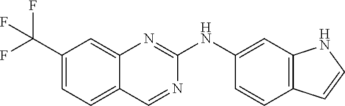 [SMILES: FC(F)(F)c1ccc2cnc(Nc3ccc4cc[nH]c4c3)nc2c1] |
| 433 | 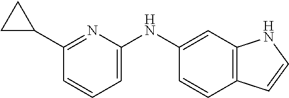 [SMILES: c1cc(Nc2ccc3cc[nH]c3c2)nc(C2CC2)c1] |
| 434 | 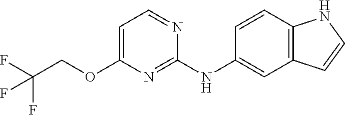 [SMILES: FC(F)(F)COc1ccnc(Nc2ccc3[nH]ccc3c2)n1] |
| 435 | 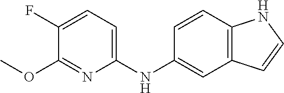 [SMILES: COc1nc(Nc2ccc3[nH]ccc3c2)ccc1F] |
| 436 | 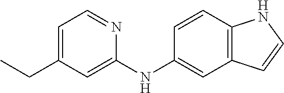 [SMILES: CCc1ccnc(Nc2ccc3[nH]ccc3c2)c1] |
| 437 | 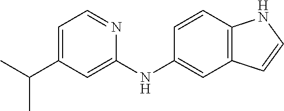 [SMILES: CC(C)c1ccnc(Nc2ccc3[nH]ccc3c2)c1] |
| 438 | 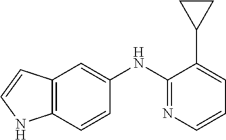 [SMILES: c1cnc(Nc2ccc3[nH]ccc3c2)c(C2CC2)c1] |
| 439 | 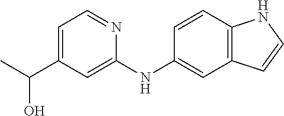 [SMILES: CC(O)c1ccnc(Nc2ccc3[nH]ccc3c2)c1] |
| 440 | 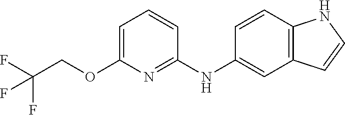 [SMILES: FC(F)(F)COc1cccc(Nc2ccc3[nH]ccc3c2)n1] |
| 441 | 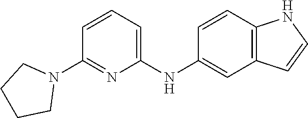 [SMILES: c1cc(Nc2ccc3[nH]ccc3c2)nc(N2CCCC2)c1] |
| 442 | 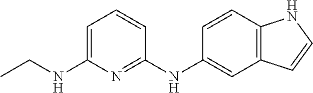 [SMILES: CCNc1cccc(Nc2ccc3[nH]ccc3c2)n1] |
| 443 | 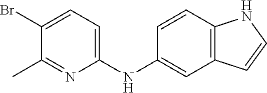 [SMILES: Cc1nc(Nc2ccc3[nH]ccc3c2)ccc1Br] |
| 444 | 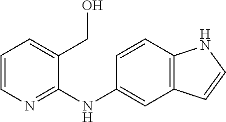 [SMILES: OCc1cccnc1Nc1ccc2[nH]ccc2c1] |
| 445 | 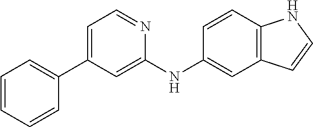 [SMILES: c1ccc(-c2ccnc(Nc3ccc4[nH]ccc4c3)c2)cc1] |
| 446 | 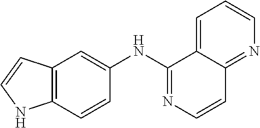 [SMILES: c1cnc2ccnc(Nc3ccc4[nH]ccc4c3)c2c1] |
| 447 | 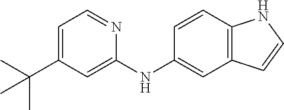 [SMILES: CC(C)(C)c1ccnc(Nc2ccc3[nH]ccc3c2)c1] |
| 448 | 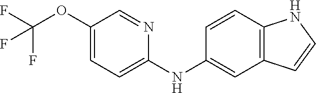 [SMILES: FC(F)(F)Oc1ccc(Nc2ccc3[nH]ccc3c2)nc1] |
| 449 | 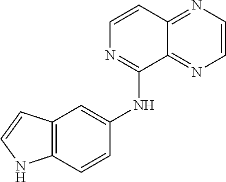 [SMILES: c1cc2nccnc2c(Nc2ccc3[nH]ccc3c2)n1] |
| 450 | 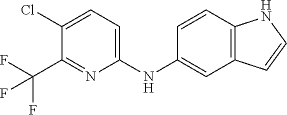 [SMILES: FC(F)(F)c1nc(Nc2ccc3[nH]ccc3c2)ccc1Cl] |
| 451 | 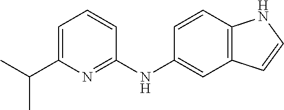 [SMILES: CC(C)c1cccc(Nc2ccc3[nH]ccc3c2)n1] |
| 452 | 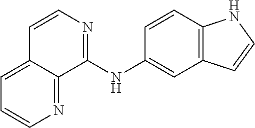 [SMILES: c1cnc2c(Nc3ccc4[nH]ccc4c3)nccc2c1] |
| 453 | 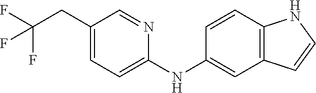 [SMILES: FC(F)(F)Cc1ccc(Nc2ccc3[nH]ccc3c2)nc1] |
| 454 | 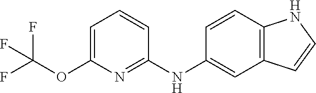 [SMILES: FC(F)(F)Oc1cccc(Nc2ccc3[nH]ccc3c2)n1] |
| 455 | 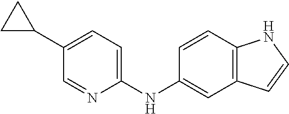 [SMILES: c1cc2cc(Nc3ccc(C4CC4)cn3)ccc2[nH]1] |
| 456 | 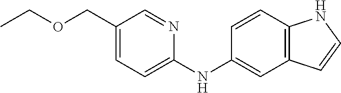 [SMILES: CCOCc1ccc(Nc2ccc3[nH]ccc3c2)nc1] |
| 457 | 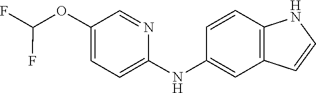 [SMILES: FC(F)Oc1ccc(Nc2ccc3[nH]ccc3c2)nc1] |
| 458 | 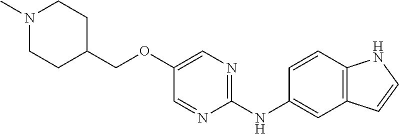 [SMILES: CN1CCC(COc2cnc(Nc3ccc4[nH]ccc4c3)nc2)CC1] |
| 459 | 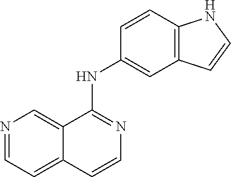 [SMILES: c1cc2ccnc(Nc3ccc4[nH]ccc4c3)c2cn1] |
| 460 | 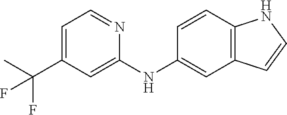 [SMILES: CC(F)(F)c1ccnc(Nc2ccc3[nH]ccc3c2)c1] |
| 461 | 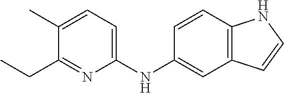 [SMILES: CCc1nc(Nc2ccc3[nH]ccc3c2)ccc1C] |
| 462 | 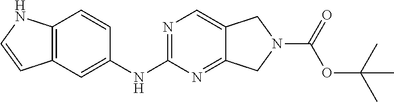 [SMILES: CC(C)(C)OC(=O)N1Cc2cnc(Nc3ccc4[nH]ccc4c3)nc2C1] |
| 463 | 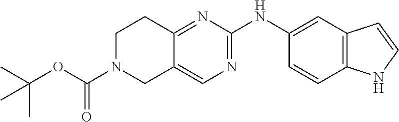 [SMILES: CC(C)(C)OC(=O)N1CCc2nc(Nc3ccc4[nH]ccc4c3)ncc2C1] |
| 464 | 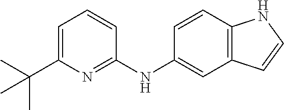 [SMILES: CC(C)(C)c1cccc(Nc2ccc3[nH]ccc3c2)n1] |
| 465 | 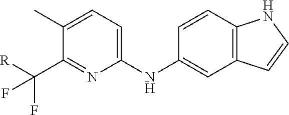 |
| 466 | 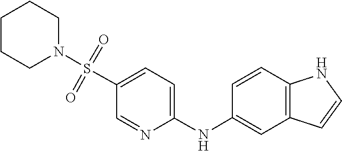 [SMILES: O=S(=O)(c1ccc(Nc2ccc3[nH]ccc3c2)nc1)N1CCCCC1] |
| 467 | 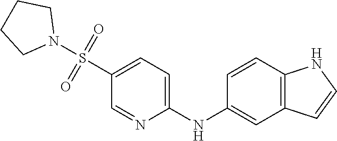 [SMILES: O=S(=O)(c1ccc(Nc2ccc3[nH]ccc3c2)nc1)N1CCCC1] |
| 468 | 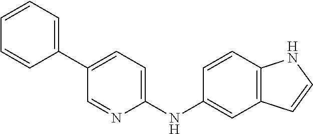 [SMILES: c1ccc(-c2ccc(Nc3ccc4[nH]ccc4c3)nc2)cc1] |
| 469 | 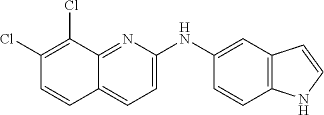 [SMILES: Clc1ccc2ccc(Nc3ccc4[nH]ccc4c3)nc2c1Cl] |
| 470 | 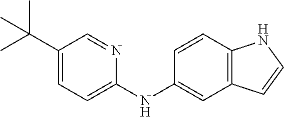 [SMILES: CC(C)(C)c1ccc(Nc2ccc3[nH]ccc3c2)nc1] |
| 471 | 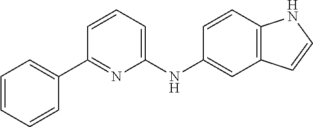 [SMILES: c1ccc(-c2cccc(Nc3ccc4[nH]ccc4c3)n2)cc1] |
| 472 | 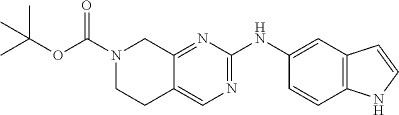 [SMILES: CC(C)(C)OC(=O)N1CCc2cnc(Nc3ccc4[nH]ccc4c3)nc2C1] |
| 473 | 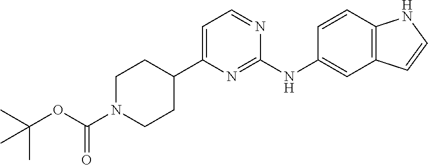 [SMILES: CC(C)(C)OC(=O)N1CCC(c2ccnc(Nc3ccc4[nH]ccc4c3)n2)CC1] |
| 474 | 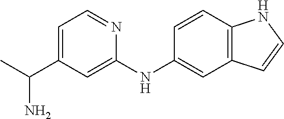 [SMILES: CC(N)c1ccnc(Nc2ccc3[nH]ccc3c2)c1] |
| 475 | 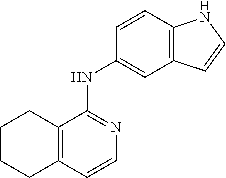 [SMILES: c1cc2c(c(Nc3ccc4[nH]ccc4c3)n1)CCCC2] |
| 476 | 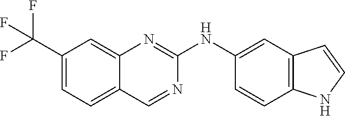 [SMILES: FC(F)(F)c1ccc2cnc(Nc3ccc4[nH]ccc4c3)nc2c1] |
| 477 | 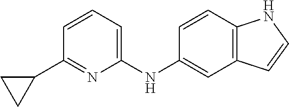 [SMILES: c1cc(Nc2ccc3[nH]ccc3c2)nc(C2CC2)c1] |
| 478 | 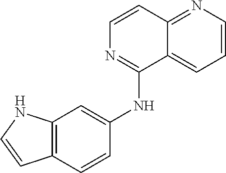 [SMILES: c1cnc2ccnc(Nc3ccc4cc[nH]c4c3)c2c1] |
| 479 | 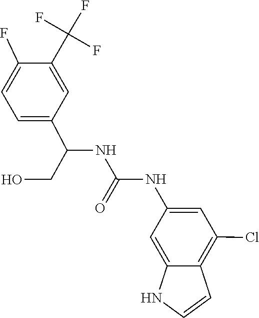 [SMILES: O=C(Nc1cc(Cl)c2cc[nH]c2c1)NC(CO)c1ccc(F)c(C(F)(F)F)c1] |
| 480 | 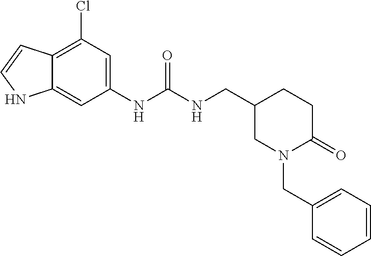 [SMILES: O=C(NCC1CCC(=O)N(Cc2ccccc2)C1)Nc1cc(Cl)c2cc[nH]c2c1] |
| 481 | 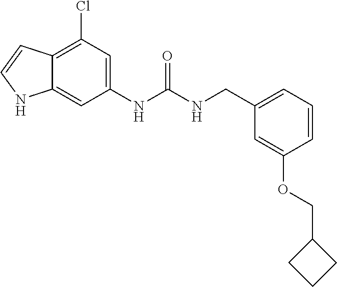 [SMILES: O=C(NCc1cccc(OCC2CCC2)c1)Nc1cc(Cl)c2cc[nH]c2c1] |
| 482 | 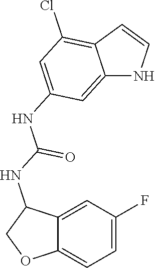 [SMILES: O=C(Nc1cc(Cl)c2cc[nH]c2c1)NC1COc2ccc(F)cc21] |
| 483 | 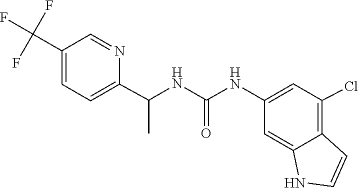 [SMILES: CC(NC(=O)Nc1cc(Cl)c2cc[nH]c2c1)c1ccc(C(F)(F)F)cn1] |
| 484 | 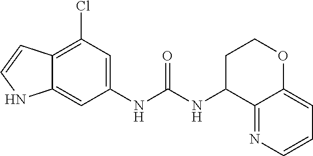 [SMILES: O=C(Nc1cc(Cl)c2cc[nH]c2c1)NC1CCOc2cccnc21] |
| 485 | 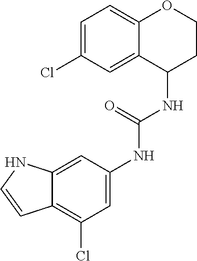 [SMILES: O=C(Nc1cc(Cl)c2cc[nH]c2c1)NC1CCOc2ccc(Cl)cc21] |
| 486 | 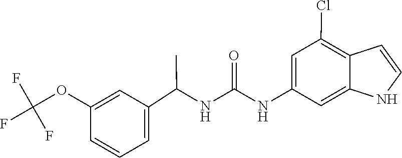 [SMILES: CC(NC(=O)Nc1cc(Cl)c2cc[nH]c2c1)c1cccc(OC(F)(F)F)c1] |
| 487 | 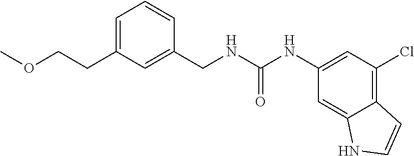 [SMILES: COCCc1cccc(CNC(=O)Nc2cc(Cl)c3cc[nH]c3c2)c1] |
| 488 | 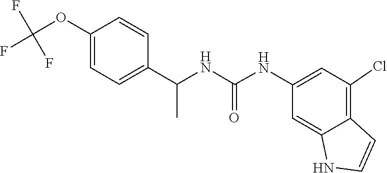 [SMILES: CC(NC(=O)Nc1cc(Cl)c2cc[nH]c2c1)c1ccc(OC(F)(F)F)cc1] |
| 489 | 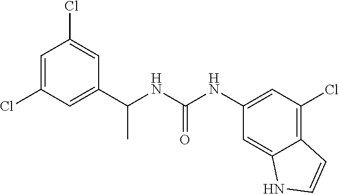 [SMILES: CC(NC(=O)Nc1cc(Cl)c2cc[nH]c2c1)c1cc(Cl)cc(Cl)c1] |
| 490 | 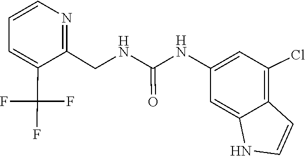 [SMILES: O=C(NCc1ncccc1C(F)(F)F)Nc1cc(Cl)c2cc[nH]c2c1] |
| 491 | 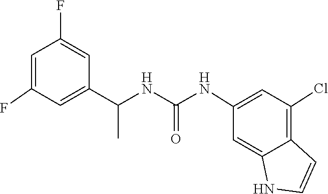 [SMILES: CC(NC(=O)Nc1cc(Cl)c2cc[nH]c2c1)c1cc(F)cc(F)c1] |
| 492 | 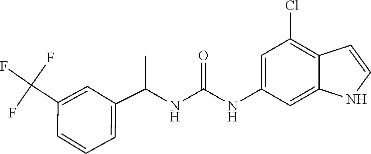 [SMILES: CC(NC(=O)Nc1cc(Cl)c2cc[nH]c2c1)c1cccc(C(F)(F)F)c1] |
| 493 | 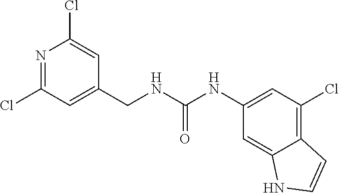 [SMILES: O=C(NCc1cc(Cl)nc(Cl)c1)Nc1cc(Cl)c2cc[nH]c2c1] |
| 494 | 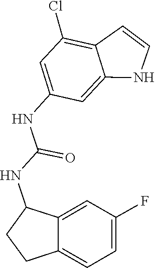 [SMILES: O=C(Nc1cc(Cl)c2cc[nH]c2c1)NC1CCc2ccc(F)cc21] |
| 495 | 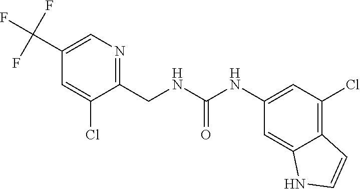 [SMILES: O=C(NCc1ncc(C(F)(F)F)cc1Cl)Nc1cc(Cl)c2cc[nH]c2c1] |
| 496 | 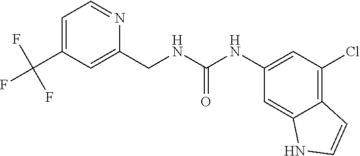 [SMILES: O=C(NCc1cc(C(F)(F)F)ccn1)Nc1cc(Cl)c2cc[nH]c2c1] |
| 497 | 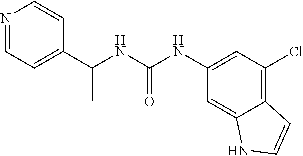 [SMILES: CC(NC(=O)Nc1cc(Cl)c2cc[nH]c2c1)c1ccncc1] |
| 498 | 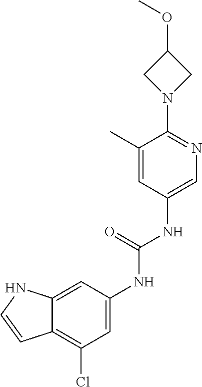 [SMILES: COC1CN(c2ncc(NC(=O)Nc3cc(Cl)c4cc[nH]c4c3)cc2C)C1] |
| 499 | 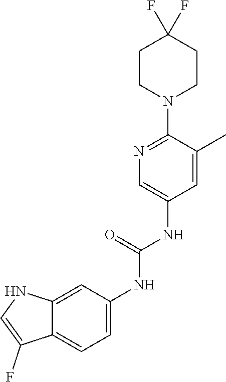 [SMILES: Cc1cc(NC(=O)Nc2ccc3c(F)c[nH]c3c2)cnc1N1CCC(F)(F)CC1] |
| 500 | 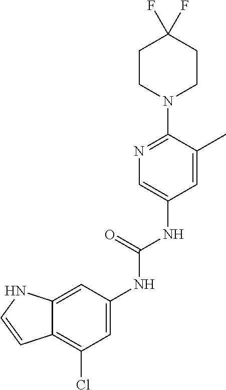 [SMILES: Cc1cc(NC(=O)Nc2cc(Cl)c3cc[nH]c3c2)cnc1N1CCC(F)(F)CC1] |
| 501 | 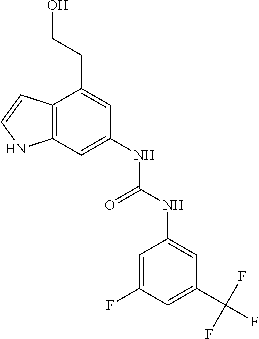 [SMILES: O=C(Nc1cc(F)cc(C(F)(F)F)c1)Nc1cc(CCO)c2cc[nH]c2c1] |
| 502 | 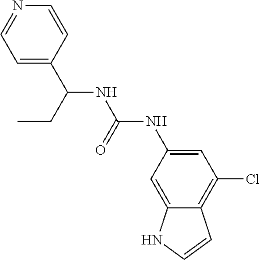 [SMILES: CCC(NC(=O)Nc1cc(Cl)c2cc[nH]c2c1)c1ccncc1] |
| 503 | 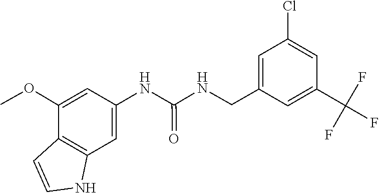 [SMILES: COc1cc(NC(=O)NCc2cc(Cl)cc(C(F)(F)F)c2)cc2[nH]ccc12] |
| 504 | 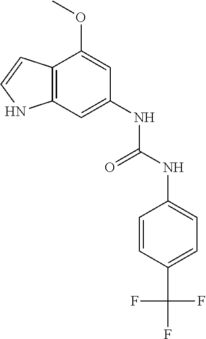 [SMILES: COc1cc(NC(=O)Nc2ccc(C(F)(F)F)cc2)cc2[nH]ccc12] |
| 505 | 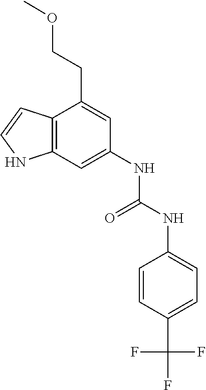 [SMILES: COCCc1cc(NC(=O)Nc2ccc(C(F)(F)F)cc2)cc2[nH]ccc12] |
| 506 | 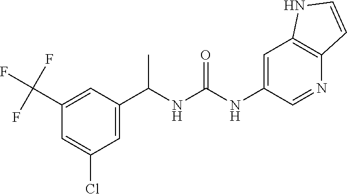 [SMILES: CC(NC(=O)Nc1cnc2cc[nH]c2c1)c1cc(Cl)cc(C(F)(F)F)c1] |
| 507 | 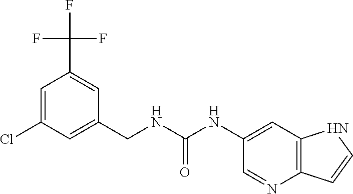 [SMILES: O=C(NCc1cc(Cl)cc(C(F)(F)F)c1)Nc1cnc2cc[nH]c2c1] |
| 508 | 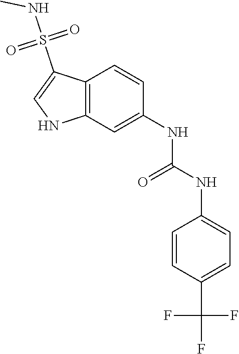 [SMILES: CNS(=O)(=O)c1c[nH]c2cc(NC(=O)Nc3ccc(C(F)(F)F)cc3)ccc12] |
| 509 | 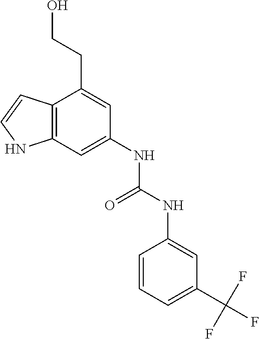 [SMILES: O=C(Nc1cccc(C(F)(F)F)c1)Nc1cc(CCO)c2cc[nH]c2c1] |
| 510 | 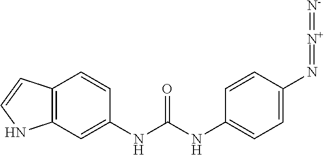 [SMILES: [N-]=[N+]=Nc1ccc(NC(=O)Nc2ccc3cc[nH]c3c2)cc1] |
| 511 | 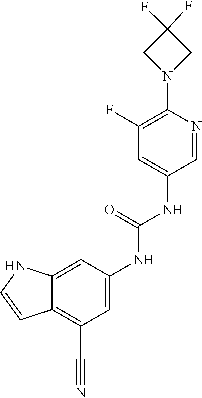 [SMILES: N#Cc1cc(NC(=O)Nc2cnc(N3CC(F)(F)C3)c(F)c2)cc2[nH]ccc12] |
| 512 | 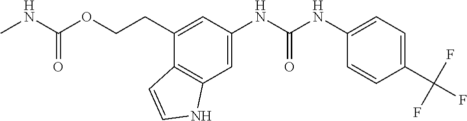 [SMILES: CNC(=O)OCCc1cc(NC(=O)Nc2ccc(C(F)(F)F)cc2)cc2[nH]ccc12] |
| 513 | 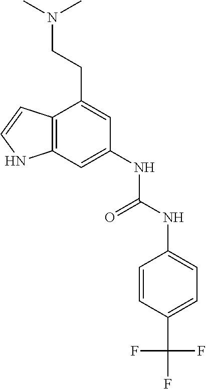 [SMILES: CN(C)CCc1cc(NC(=O)Nc2ccc(C(F)(F)F)cc2)cc2[nH]ccc12] |
| 514 | 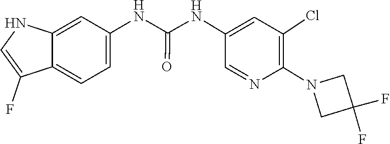 [SMILES: O=C(Nc1cnc(N2CC(F)(F)C2)c(Cl)c1)Nc1ccc2c(F)c[nH]c2c1] |
| 515 | 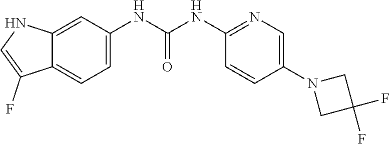 [SMILES: O=C(Nc1ccc2c(F)c[nH]c2c1)Nc1ccc(N2CC(F)(F)C2)cn1] |
| 516 | 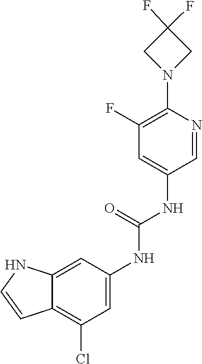 [SMILES: O=C(Nc1cnc(N2CC(F)(F)C2)c(F)c1)Nc1cc(Cl)c2cc[nH]c2c1] |
| 517 | 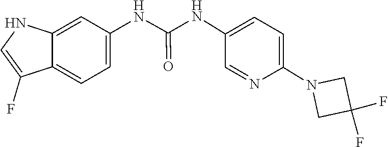 [SMILES: O=C(Nc1ccc(N2CC(F)(F)C2)nc1)Nc1ccc2c(F)c[nH]c2c1] |
| 518 | 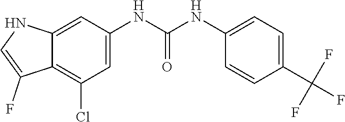 [SMILES: O=C(Nc1ccc(C(F)(F)F)cc1)Nc1cc(Cl)c2c(F)c[nH]c2c1] |
| 519 | 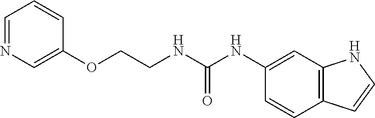 [SMILES: O=C(NCCOc1cccnc1)Nc1ccc2cc[nH]c2c1] |
| 520 | 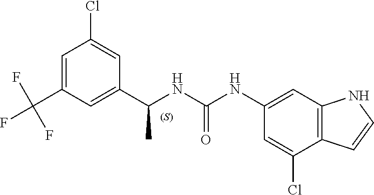 |
| 521 | 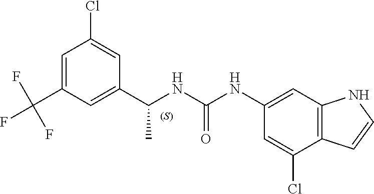 |
| 522 | 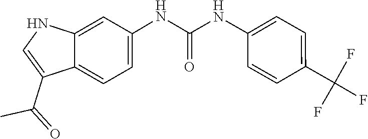 [SMILES: CC(=O)c1c[nH]c2cc(NC(=O)Nc3ccc(C(F)(F)F)cc3)ccc12] |
| 523 | 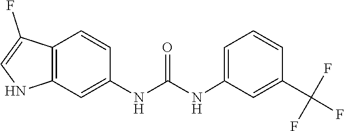 [SMILES: O=C(Nc1cccc(C(F)(F)F)c1)Nc1ccc2c(F)c[nH]c2c1] |
| 524 | 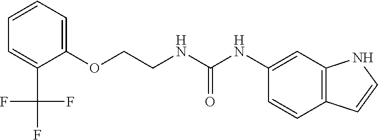 [SMILES: O=C(NCCOc1ccccc1C(F)(F)F)Nc1ccc2cc[nH]c2c1] |
| 525 | 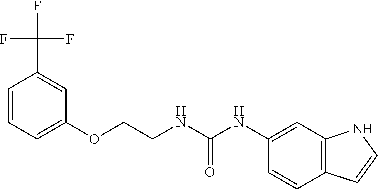 [SMILES: O=C(NCCOc1cccc(C(F)(F)F)c1)Nc1ccc2cc[nH]c2c1] |
| 526 | 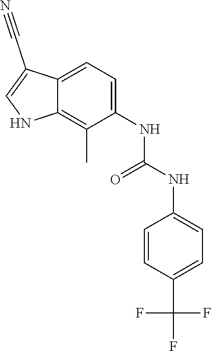 [SMILES: Cc1c(NC(=O)Nc2ccc(C(F)(F)F)cc2)ccc2c(C#N)c[nH]c12] |
| 527 | 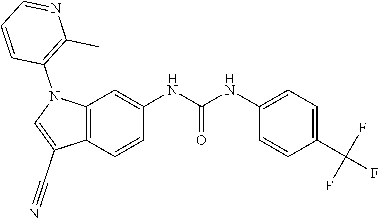 [SMILES: Cc1ncccc1-n1cc(C#N)c2ccc(NC(=O)Nc3ccc(C(F)(F)F)cc3)cc21] |
| 528 | 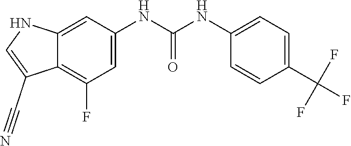 [SMILES: N#Cc1c[nH]c2cc(NC(=O)Nc3ccc(C(F)(F)F)cc3)cc(F)c12] |
| 529 | 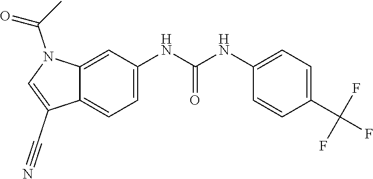 [SMILES: CC(=O)n1cc(C#N)c2ccc(NC(=O)Nc3ccc(C(F)(F)F)cc3)cc21] |
| 530 | 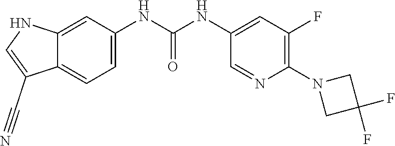 [SMILES: N#Cc1c[nH]c2cc(NC(=O)Nc3cnc(N4CC(F)(F)C4)c(F)c3)ccc12] |
| 531 | 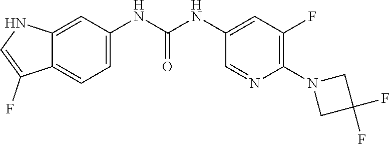 [SMILES: O=C(Nc1cnc(N2CC(F)(F)C2)c(F)c1)Nc1ccc2c(F)c[nH]c2c1] |
| 532 | 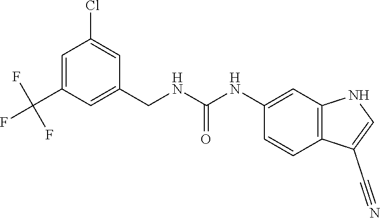 [SMILES: N#Cc1c[nH]c2cc(NC(=O)NCc3cc(Cl)cc(C(F)(F)F)c3)ccc12] |
| 533 | 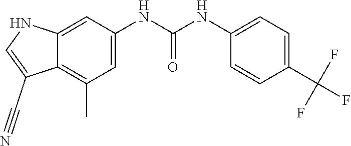 [SMILES: Cc1cc(NC(=O)Nc2ccc(C(F)(F)F)cc2)cc2[nH]cc(C#N)c12] |
| 534 | 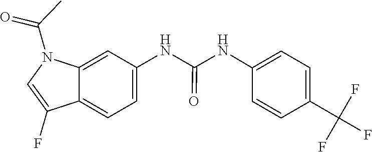 [SMILES: CC(=O)n1cc(F)c2ccc(NC(=O)Nc3ccc(C(F)(F)F)cc3)cc21] |
| 535 | 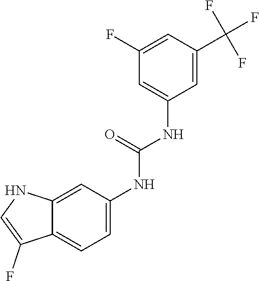 [SMILES: O=C(Nc1cc(F)cc(C(F)(F)F)c1)Nc1ccc2c(F)c[nH]c2c1] |
| 536 | 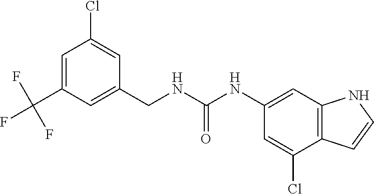 [SMILES: O=C(NCc1cc(Cl)cc(C(F)(F)F)c1)Nc1cc(Cl)c2cc[nH]c2c1] |
| 537 | 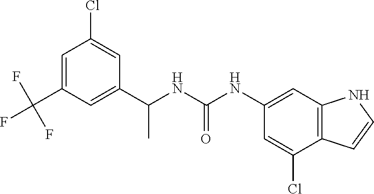 [SMILES: CC(NC(=O)Nc1cc(Cl)c2cc[nH]c2c1)c1cc(Cl)cc(C(F)(F)F)c1] |
| 538 | 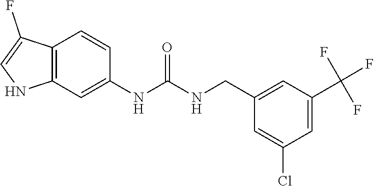 [SMILES: O=C(NCc1cc(Cl)cc(C(F)(F)F)c1)Nc1ccc2c(F)c[nH]c2c1] |
| 539 | 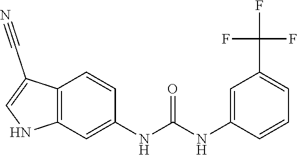 [SMILES: N#Cc1c[nH]c2cc(NC(=O)Nc3cccc(C(F)(F)F)c3)ccc12] |
| 540 | 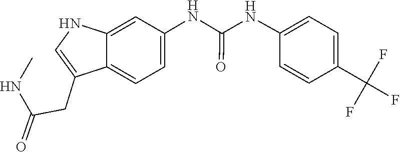 [SMILES: CNC(=O)Cc1c[nH]c2cc(NC(=O)Nc3ccc(C(F)(F)F)cc3)ccc12] |
| 541 | 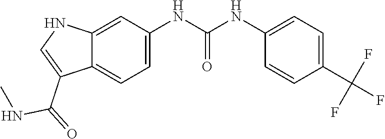 [SMILES: CNC(=O)c1c[nH]c2cc(NC(=O)Nc3ccc(C(F)(F)F)cc3)ccc12] |
| 542 | 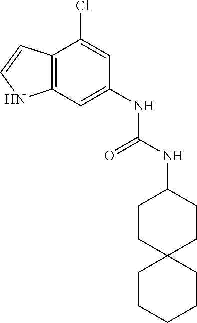 [SMILES: O=C(Nc1cc(Cl)c2cc[nH]c2c1)NC1CCC2(CCCCC2)CC1] |
| 543 | 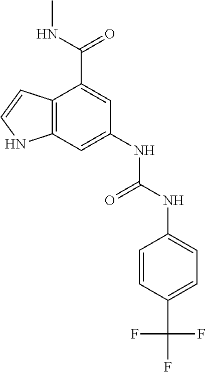 [SMILES: CNC(=O)c1cc(NC(=O)Nc2ccc(C(F)(F)F)cc2)cc2[nH]ccc12] |
| 544 | 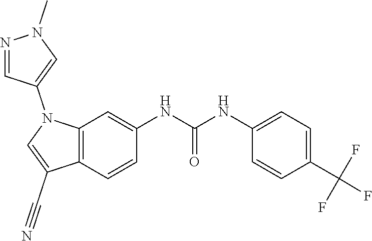 [SMILES: Cn1cc(-n2cc(C#N)c3ccc(NC(=O)Nc4ccc(C(F)(F)F)cc4)cc32)cn1] |
| 545 | 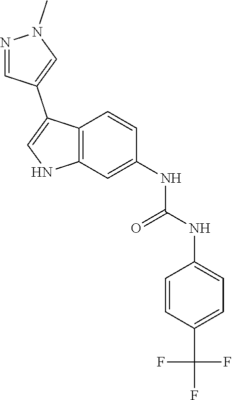 [SMILES: Cn1cc(-c2c[nH]c3cc(NC(=O)Nc4ccc(C(F)(F)F)cc4)ccc23)cn1] |
| 546 | 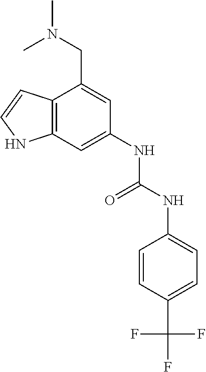 [SMILES: CN(C)Cc1cc(NC(=O)Nc2ccc(C(F)(F)F)cc2)cc2[nH]ccc12] |
| 547 | 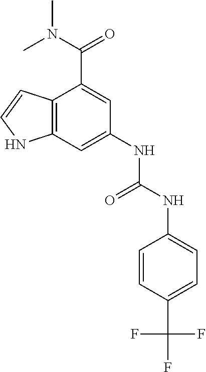 [SMILES: CN(C)C(=O)c1cc(NC(=O)Nc2ccc(C(F)(F)F)cc2)cc2[nH]ccc12] |
| 548 | 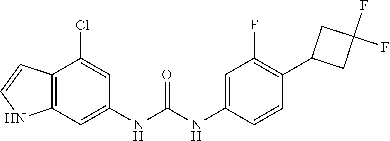 [SMILES: O=C(Nc1ccc(C2CC(F)(F)C2)c(F)c1)Nc1cc(Cl)c2cc[nH]c2c1] |
| 549 | 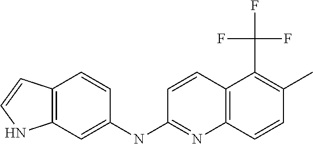 |
| 550 | 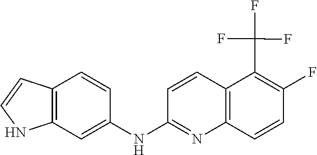 [SMILES: Fc1ccc2nc(Nc3ccc4cc[nH]c4c3)ccc2c1C(F)(F)F] |
| 551 | 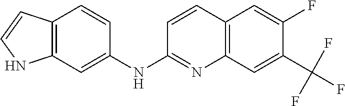 [SMILES: Fc1cc2ccc(Nc3ccc4cc[nH]c4c3)nc2cc1C(F)(F)F] |
| 552 | 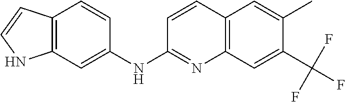 [SMILES: Cc1cc2ccc(Nc3ccc4cc[nH]c4c3)nc2cc1C(F)(F)F] |
| 553 | 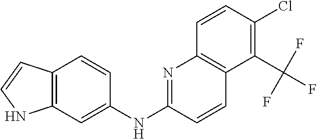 [SMILES: FC(F)(F)c1c(Cl)ccc2nc(Nc3ccc4cc[nH]c4c3)ccc12] |
| 554 | 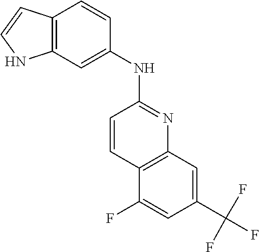 [SMILES: Fc1cc(C(F)(F)F)cc2nc(Nc3ccc4cc[nH]c4c3)ccc12] |
| 555 | 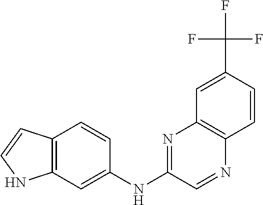 [SMILES: FC(F)(F)c1ccc2ncc(Nc3ccc4cc[nH]c4c3)nc2c1] |
| 556 | 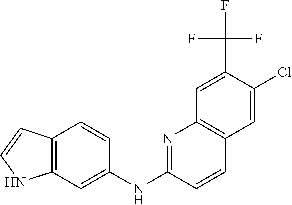 [SMILES: FC(F)(F)c1cc2nc(Nc3ccc4cc[nH]c4c3)ccc2cc1Cl] |
| 557 | 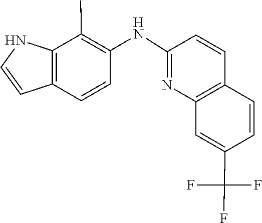 [SMILES: Cc1c(Nc2ccc3ccc(C(F)(F)F)cc3n2)ccc2cc[nH]c12] |
| 558 | 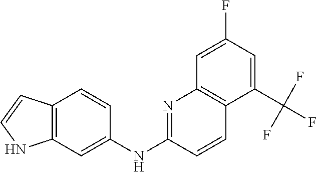 [SMILES: Fc1cc(C(F)(F)F)c2ccc(Nc3ccc4cc[nH]c4c3)nc2c1] |
| 559 | 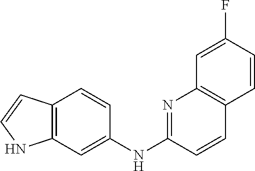 [SMILES: Fc1ccc2ccc(Nc3ccc4cc[nH]c4c3)nc2c1] |
| 560 | 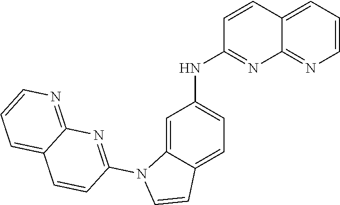 [SMILES: c1cnc2nc(Nc3ccc4ccn(-c5ccc6cccnc6n5)c4c3)ccc2c1] |
| 561 | 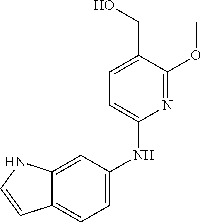 [SMILES: COc1nc(Nc2ccc3cc[nH]c3c2)ccc1CO] |
| 562 | 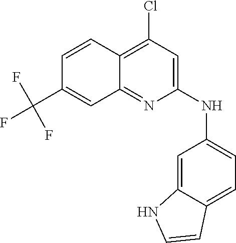 [SMILES: FC(F)(F)c1ccc2c(Cl)cc(Nc3ccc4cc[nH]c4c3)nc2c1] |
| 563 | 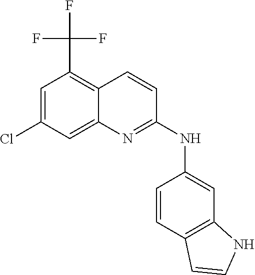 [SMILES: FC(F)(F)c1cc(Cl)cc2nc(Nc3ccc4cc[nH]c4c3)ccc12] |
| 564 | 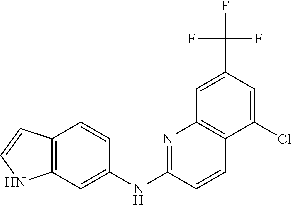 [SMILES: FC(F)(F)c1cc(Cl)c2ccc(Nc3ccc4cc[nH]c4c3)nc2c1] |
| 565 | 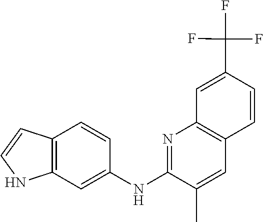 [SMILES: Cc1cc2ccc(C(F)(F)F)cc2nc1Nc1ccc2cc[nH]c2c1] |
| 566 | 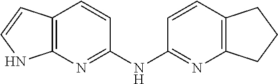 [SMILES: c1cc2ccc(Nc3ccc4c(n3)CCC4)nc2[nH]1] |
| 567 | 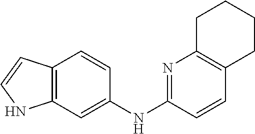 [SMILES: c1cc2ccc(Nc3ccc4c(n3)CCCC4)cc2[nH]1] |
| 568 | 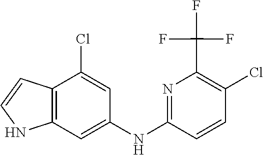 [SMILES: FC(F)(F)c1nc(Nc2cc(Cl)c3cc[nH]c3c2)ccc1Cl] |
| 569 | 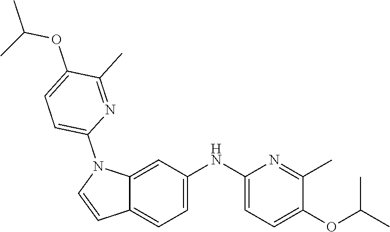 [SMILES: Cc1nc(Nc2ccc3ccn(-c4ccc(OC(C)C)c(C)n4)c3c2)ccc1OC(C)C] |
| 570 | 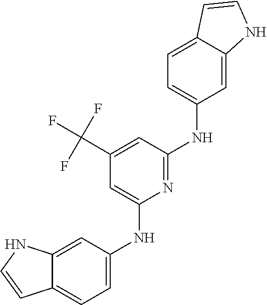 [SMILES: FC(F)(F)c1cc(Nc2ccc3cc[nH]c3c2)nc(Nc2ccc3cc[nH]c3c2)c1] |
| 571 | 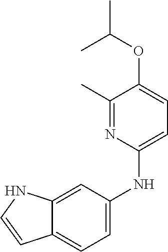 [SMILES: Cc1nc(Nc2ccc3cc[nH]c3c2)ccc1OC(C)C] |
| 572 | 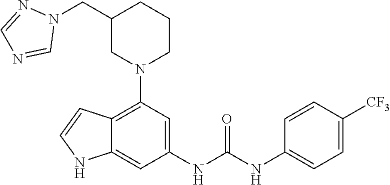 [SMILES: O=C(Nc1ccc(C(F)(F)F)cc1)Nc1cc(N2CCCC(Cn3cncn3)C2)c2cc[nH]c2c1] |
| 573 | 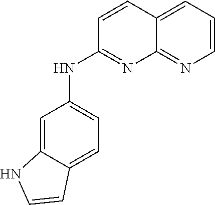 [SMILES: c1cnc2nc(Nc3ccc4cc[nH]c4c3)ccc2c1] |
| 574 | 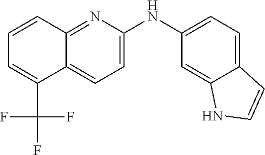 [SMILES: FC(F)(F)c1cccc2nc(Nc3ccc4cc[nH]c4c3)ccc12] |
| 575 | 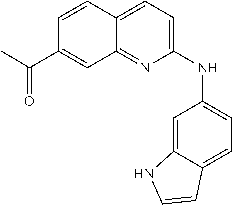 [SMILES: CC(=O)c1ccc2ccc(Nc3ccc4cc[nH]c4c3)nc2c1] |
| 576 | 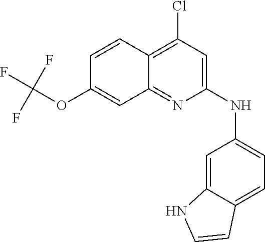 [SMILES: FC(F)(F)Oc1ccc2c(Cl)cc(Nc3ccc4cc[nH]c4c3)nc2c1] |
| 577 | 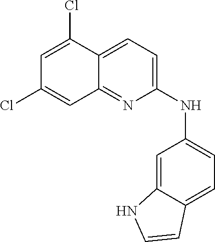 [SMILES: Clc1cc(Cl)c2ccc(Nc3ccc4cc[nH]c4c3)nc2c1] |
| 578 | 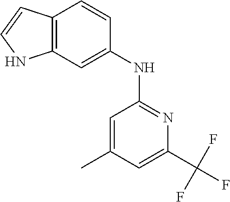 [SMILES: Cc1cc(Nc2ccc3cc[nH]c3c2)nc(C(F)(F)F)c1] |
| 579 | 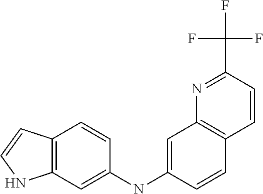 |
| 580 | 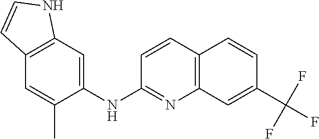 [SMILES: Cc1cc2cc[nH]c2cc1Nc1ccc2ccc(C(F)(F)F)cc2n1] |
| 581 | 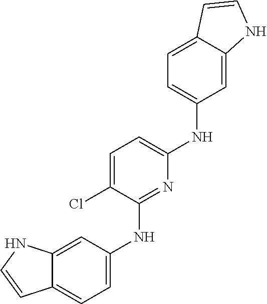 [SMILES: Clc1ccc(Nc2ccc3cc[nH]c3c2)nc1Nc1ccc2cc[nH]c2c1] |
| 582 | 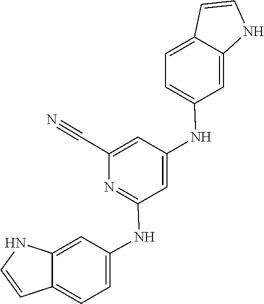 [SMILES: N#Cc1cc(Nc2ccc3cc[nH]c3c2)cc(Nc2ccc3cc[nH]c3c2)n1] |
| 583 | 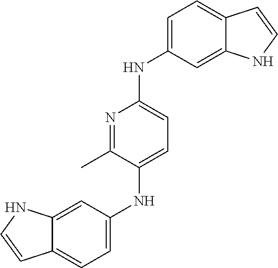 [SMILES: Cc1nc(Nc2ccc3cc[nH]c3c2)ccc1Nc1ccc2cc[nH]c2c1] |
| 584 | 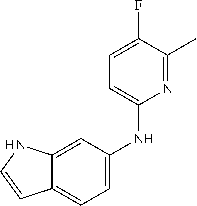 [SMILES: Cc1nc(Nc2ccc3cc[nH]c3c2)ccc1F] |
| 585 | 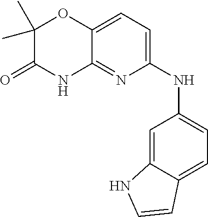 [SMILES: CC1(C)Oc2ccc(Nc3ccc4cc[nH]c4c3)nc2NC1=O] |
| 586 | 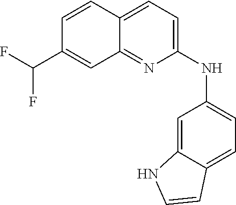 [SMILES: FC(F)c1ccc2ccc(Nc3ccc4cc[nH]c4c3)nc2c1] |
| 587 | 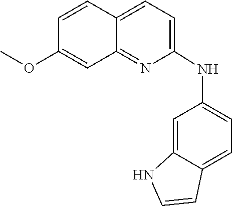 [SMILES: COc1ccc2ccc(Nc3ccc4cc[nH]c4c3)nc2c1] |
| 588 | 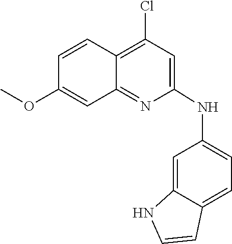 [SMILES: COc1ccc2c(Cl)cc(Nc3ccc4cc[nH]c4c3)nc2c1] |
| 589 | 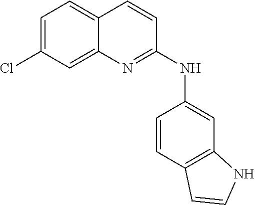 [SMILES: Clc1ccc2ccc(Nc3ccc4cc[nH]c4c3)nc2c1] |
| 590 | 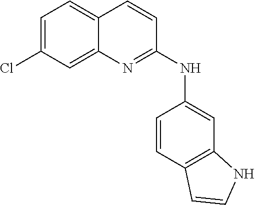 [SMILES: Clc1ccc2ccc(Nc3ccc4cc[nH]c4c3)nc2c1] |
| 591 | 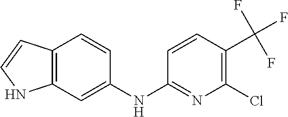 [SMILES: FC(F)(F)c1ccc(Nc2ccc3cc[nH]c3c2)nc1Cl] |
| 592 | 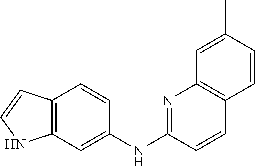 [SMILES: Cc1ccc2ccc(Nc3ccc4cc[nH]c4c3)nc2c1] |
| 593 | 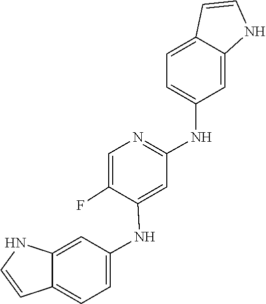 [SMILES: Fc1cnc(Nc2ccc3cc[nH]c3c2)cc1Nc1ccc2cc[nH]c2c1] |
| 594 | 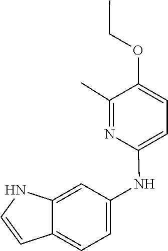 [SMILES: CCOc1ccc(Nc2ccc3cc[nH]c3c2)nc1C] |
| 595 | 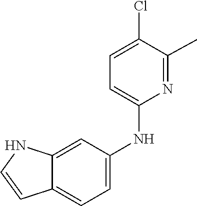 [SMILES: Cc1nc(Nc2ccc3cc[nH]c3c2)ccc1Cl] |
| 596 | 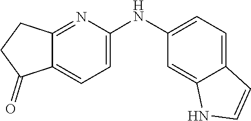 [SMILES: O=C1CCc2nc(Nc3ccc4cc[nH]c4c3)ccc21] |
| 597 | 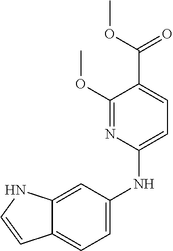 [SMILES: COC(=O)c1ccc(Nc2ccc3cc[nH]c3c2)nc1OC] |
| 598 | 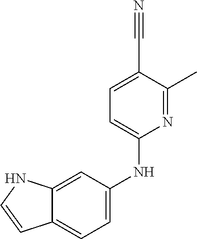 [SMILES: Cc1nc(Nc2ccc3cc[nH]c3c2)ccc1C#N] |
| 599 | 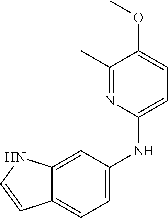 [SMILES: COc1ccc(Nc2ccc3cc[nH]c3c2)nc1C] |
| 600 | 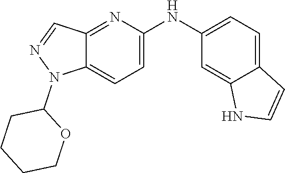 [SMILES: c1cc2ccc(Nc3ccc4c(cnn4C4CCCCO4)n3)cc2[nH]1] |
| 601 | 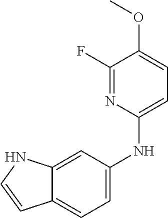 [SMILES: COc1ccc(Nc2ccc3cc[nH]c3c2)nc1F] |
| 602 | 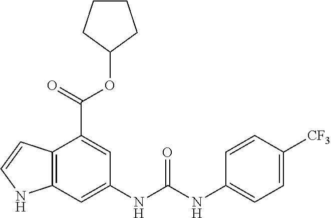 [SMILES: O=C(Nc1ccc(C(F)(F)F)cc1)Nc1cc(C(=O)OC2CCCC2)c2cc[nH]c2c1] |
| 603 | 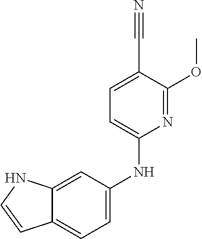 [SMILES: COc1nc(Nc2ccc3cc[nH]c3c2)ccc1C#N] |
| 604 | 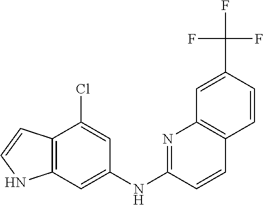 [SMILES: FC(F)(F)c1ccc2ccc(Nc3cc(Cl)c4cc[nH]c4c3)nc2c1] |
| 605 | 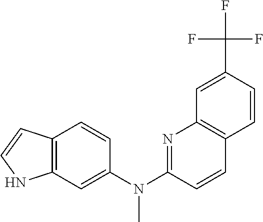 |
| 606 | 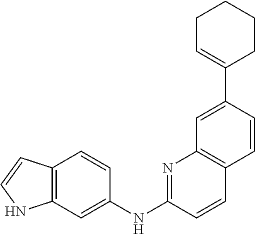 [SMILES: C1=C(c2ccc3ccc(Nc4ccc5cc[nH]c5c4)nc3c2)CCCC1] |
| 607 | 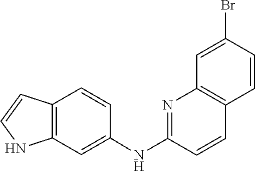 [SMILES: Brc1ccc2ccc(Nc3ccc4cc[nH]c4c3)nc2c1] |
| 608 | 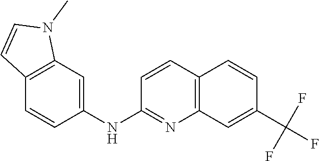 [SMILES: Cn1ccc2ccc(Nc3ccc4ccc(C(F)(F)F)cc4n3)cc21] |
| 609 | 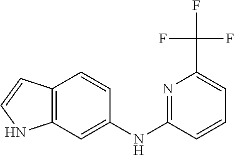 [SMILES: FC(F)(F)c1cccc(Nc2ccc3cc[nH]c3c2)n1] |
| 611 | 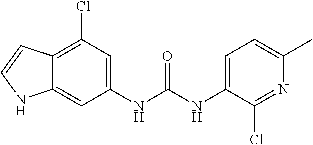 [SMILES: Cc1ccc(NC(=O)Nc2cc(Cl)c3cc[nH]c3c2)c(Cl)n1] |
| 612 | 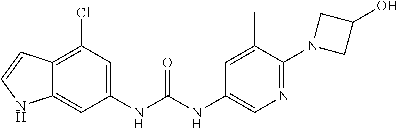 [SMILES: Cc1cc(NC(=O)Nc2cc(Cl)c3cc[nH]c3c2)cnc1N1CC(O)C1] |
| 613 | 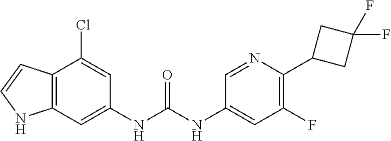 [SMILES: O=C(Nc1cnc(C2CC(F)(F)C2)c(F)c1)Nc1cc(Cl)c2cc[nH]c2c1] |
| 614 | 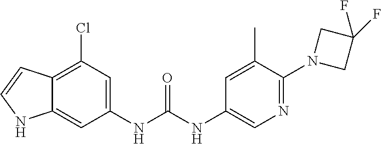 [SMILES: Cc1cc(NC(=O)Nc2cc(Cl)c3cc[nH]c3c2)cnc1N1CC(F)(F)C1] |
| 615 | 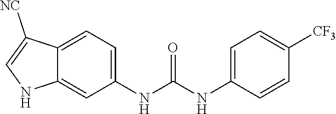 [SMILES: N#Cc1c[nH]c2cc(NC(=O)Nc3ccc(C(F)(F)F)cc3)ccc12] |
| 616 | 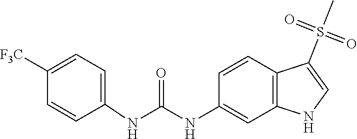 [SMILES: CS(=O)(=O)c1c[nH]c2cc(NC(=O)Nc3ccc(C(F)(F)F)cc3)ccc12] |
| 617 | 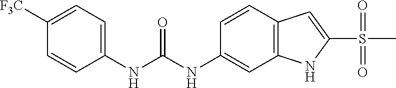 [SMILES: CS(=O)(=O)c1cc2ccc(NC(=O)Nc3ccc(C(F)(F)F)cc3)cc2[nH]1] |
| 618 | 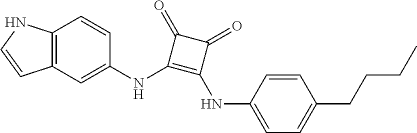 [SMILES: CCCCc1ccc(Nc2c(Nc3ccc4[nH]ccc4c3)c(=O)c2=O)cc1] |
| 619 | 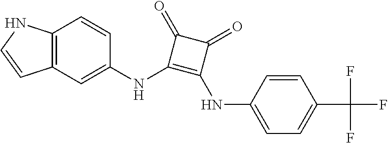 [SMILES: O=c1c(Nc2ccc(C(F)(F)F)cc2)c(Nc2ccc3[nH]ccc3c2)c1=O] |
| 620 | 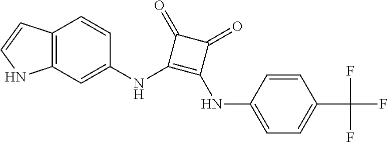 [SMILES: O=c1c(Nc2ccc(C(F)(F)F)cc2)c(Nc2ccc3cc[nH]c3c2)c1=O] |
| 621 | 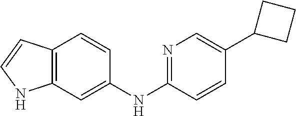 [SMILES: c1cc2ccc(Nc3ccc(C4CCC4)cn3)cc2[nH]1] |
| 622 | 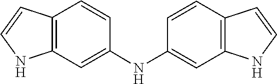 [SMILES: c1cc2ccc(Nc3ccc4cc[nH]c4c3)cc2[nH]1] |
| 623 | 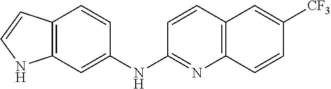 [SMILES: FC(F)(F)c1ccc2nc(Nc3ccc4cc[nH]c4c3)ccc2c1] |
| 624 | 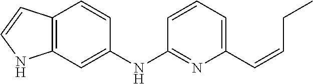 [SMILES: CC/C=C\c1cccc(Nc2ccc3cc[nH]c3c2)n1] |
| 625 | 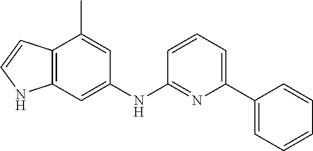 [SMILES: Cc1cc(Nc2cccc(-c3ccccc3)n2)cc2[nH]ccc12] |
| 626 | 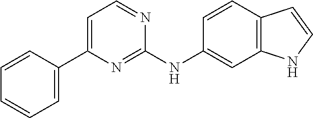 [SMILES: c1ccc(-c2ccnc(Nc3ccc4cc[nH]c4c3)n2)cc1] |
| 627 | 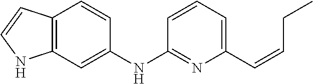 [SMILES: CC/C=C\c1cccc(Nc2ccc3cc[nH]c3c2)n1] |
| 628 | 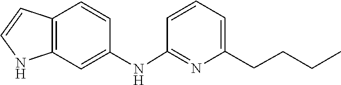 [SMILES: CCCCc1cccc(Nc2ccc3cc[nH]c3c2)n1] |
| 630 | 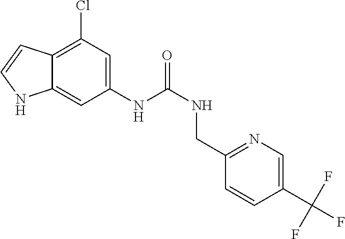 [SMILES: O=C(NCc1ccc(C(F)(F)F)cn1)Nc1cc(Cl)c2cc[nH]c2c1] |
| 631 | 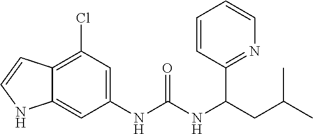 [SMILES: CC(C)CC(NC(=O)Nc1cc(Cl)c2cc[nH]c2c1)c1ccccn1] |
| 632 | 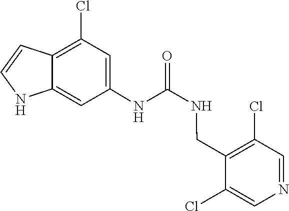 [SMILES: O=C(NCc1c(Cl)cncc1Cl)Nc1cc(Cl)c2cc[nH]c2c1] |
| 633 | 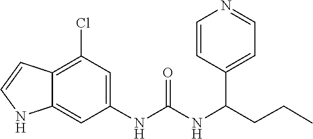 [SMILES: CCCC(NC(=O)Nc1cc(Cl)c2cc[nH]c2c1)c1ccncc1] |
| 634 | 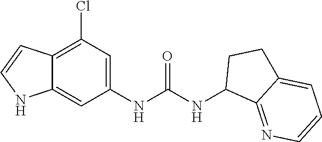 [SMILES: O=C(Nc1cc(Cl)c2cc[nH]c2c1)NC1CCc2cccnc21] |
| 635 | 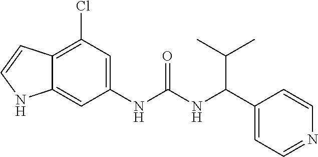 [SMILES: CC(C)C(NC(=O)Nc1cc(Cl)c2cc[nH]c2c1)c1ccncc1] |
| 636 | 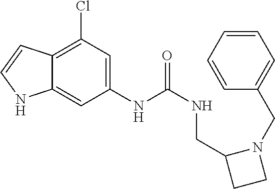 [SMILES: O=C(NCC1CCN1Cc1ccccc1)Nc1cc(Cl)c2cc[nH]c2c1] |
| 637 | 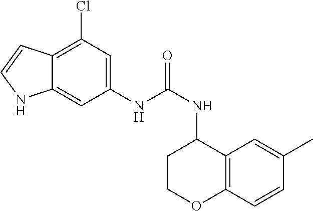 [SMILES: Cc1ccc2c(c1)C(NC(=O)Nc1cc(Cl)c3cc[nH]c3c1)CCO2] |
| 638 | 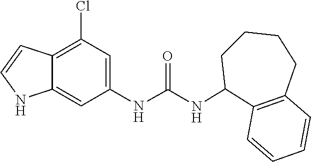 [SMILES: O=C(Nc1cc(Cl)c2cc[nH]c2c1)NC1CCCCc2ccccc21] |
| 639 | 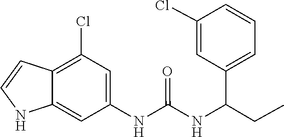 [SMILES: CCC(NC(=O)Nc1cc(Cl)c2cc[nH]c2c1)c1cccc(Cl)c1] |
| 640 | 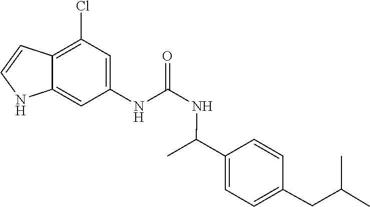 [SMILES: CC(C)Cc1ccc(C(C)NC(=O)Nc2cc(Cl)c3cc[nH]c3c2)cc1] |
| 641 | 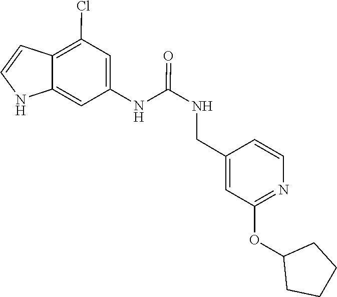 [SMILES: O=C(NCc1ccnc(OC2CCCC2)c1)Nc1cc(Cl)c2cc[nH]c2c1] |
| 642 | 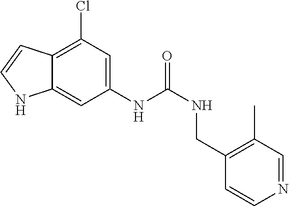 [SMILES: Cc1cnccc1CNC(=O)Nc1cc(Cl)c2cc[nH]c2c1] |
| 643 | 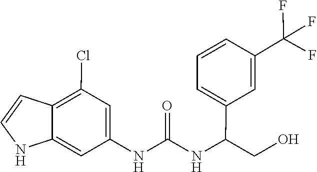 [SMILES: O=C(Nc1cc(Cl)c2cc[nH]c2c1)NC(CO)c1cccc(C(F)(F)F)c1] |
| 644 | 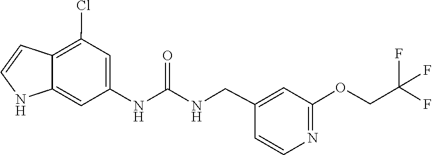 [SMILES: O=C(NCc1ccnc(OCC(F)(F)F)c1)Nc1cc(Cl)c2cc[nH]c2c1] |
| 645 | 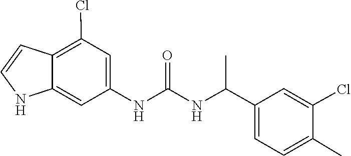 [SMILES: Cc1ccc(C(C)NC(=O)Nc2cc(Cl)c3cc[nH]c3c2)cc1Cl] |
| 646 | 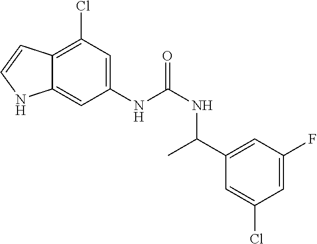 [SMILES: CC(NC(=O)Nc1cc(Cl)c2cc[nH]c2c1)c1cc(F)cc(Cl)c1] |
| 647 | 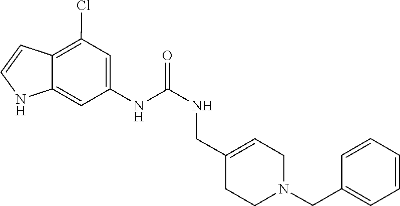 [SMILES: O=C(NCC1=CCN(Cc2ccccc2)CC1)Nc1cc(Cl)c2cc[nH]c2c1] |
| 648 | 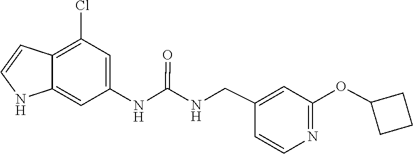 [SMILES: O=C(NCc1ccnc(OC2CCC2)c1)Nc1cc(Cl)c2cc[nH]c2c1] |
| 649 | 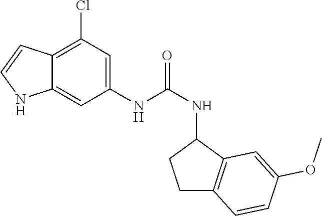 [SMILES: COc1ccc2c(c1)C(NC(=O)Nc1cc(Cl)c3cc[nH]c3c1)CC2] |
| 650 | 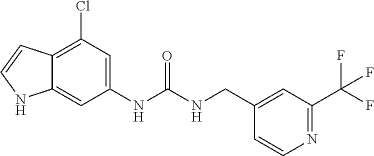 [SMILES: O=C(NCc1ccnc(C(F)(F)F)c1)Nc1cc(Cl)c2cc[nH]c2c1] |
| 651 | 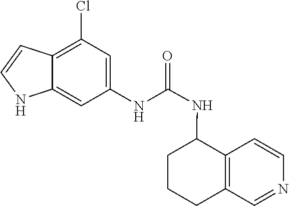 [SMILES: O=C(Nc1cc(Cl)c2cc[nH]c2c1)NC1CCCc2cnccc21] |
| 652 | 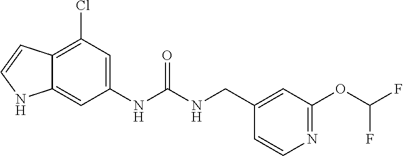 [SMILES: O=C(NCc1ccnc(OC(F)F)c1)Nc1cc(Cl)c2cc[nH]c2c1] |
| 653 | 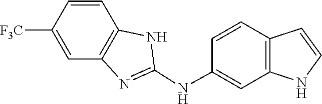 [SMILES: FC(F)(F)c1ccc2[nH]c(Nc3ccc4cc[nH]c4c3)nc2c1] |
| 654 | 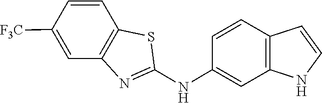 [SMILES: FC(F)(F)c1ccc2sc(Nc3ccc4cc[nH]c4c3)nc2c1] |
| 655 | 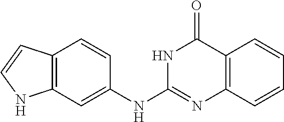 [SMILES: O=c1[nH]c(Nc2ccc3cc[nH]c3c2)nc2ccccc12] |
| 656 | 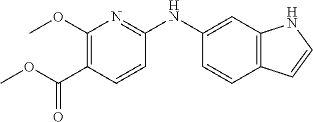 [SMILES: COC(=O)c1ccc(Nc2ccc3cc[nH]c3c2)nc1OC] |

Compounds of Formula (VII) and Table 6, and methods of making and using the same are further described in PCT/US2020/037403, filed on Jun. 12, 2020; U.S. Provisional 62/861,714, filed on Jun. 14, 2019; and U.S. Provisional 62/955,924, filed on Dec. 31, 2019, each of which is incorporated herein by reference in its entirety.

In another aspect, the STING antagonist is a compound of Formula (VIII):

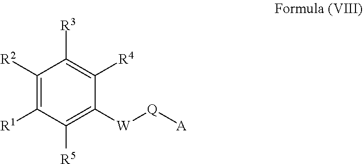

or a pharmaceutically acceptable salt thereof, wherein:
W is selected from the group consisting of:
(i) C(═O); (ii) C(═S); (iii) C(═NR^{d}); (iv) C(═NH); (v) S(O)₁₋₂; (vi) S(O)(NR^{d}); (vii) S(O)(NH); (viii) C(═C—NO₂); and (ix) C₁₋₃ alkylene optionally substituted with from 1-4 independently selected halo (e.g., F);
Q-A is defined according to (A) or (B) below:

### A

### Q is NH or N(R^{q}),

wherein R^{q} is C₁₋₆ alkyl which is optionally substituted with from 1-2 independently selected R^{a}; or
R^{q} and R⁴, taken together with the atoms connecting them, forms a ring including 5-8 ring atoms, wherein the ring includes (a) from 2-7 carbon atoms and (b) from 0-2 heteroatoms aside from Q, wherein each of the heteroatoms is independently selected from N, N(H), O, and S(O)₀₋₂.

### A is:

(i) —(Y^{A1})ₙ—Y^{A2}, wherein:
n is 0 or 1;
Y^{A1} is C₁₋₆ alkylene, which is optionally substituted with from 1-6 R^{a} and further optionally substituted with one oxo; and
Y^{A2} is:
   (a) C₃₋₂₀ cycloalkyl, which is optionally substituted with from 1-4 R^{b},
   (b) C₆₋₂₀ aryl, which is optionally substituted with from 1-4 R^{c};
   (c) heteroaryl including from 5-20 ring atoms, wherein from 1-4 ring atoms are heteroatoms, each independently selected from the group consisting of N, N(H), N(R^{d}), O, and S(O)₀₋₂, and wherein one or more of the heteroaryl ring carbon atoms are optionally substituted with from 1-4 independently selected R^{c}, or
   (d) heterocyclyl including from 3-16 ring atoms, wherein from 1-3 ring atoms are heteroatoms, each independently selected from the group consisting of N, N(H), N(R^{d}), O, and S(O)₀₋₂, and wherein one or more of the heterocyclyl ring carbon atoms are optionally substituted with from 1-4 independently selected R^{b},

### OR

(ii) C₁₋₁₀ alkyl, which is optionally substituted with from 1-6 independently selected R^{a}, or

### B

Q and A, taken together, form:

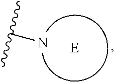

wherein [symbol] denotes point of attachment to W; and

E is heterocyclyl including from 3-16 ring atoms, wherein aside from the nitrogen atom present, from 0-3 additional ring atoms are heteroatoms, each independently selected from the group consisting of N, N(H), N(R^{d}), O, and S(O)₀₋₂, and wherein one or more of the heterocyclyl ring carbon atoms are optionally substituted with from 1-4 independently selected R^{b};

R¹ is selected from the group consisting of:
NO₂, F, SO₂R^{4A}, S(O)₁₋₂N(R^{6A})₂, CN, C(═O)R^{4A}, C(O)OR^{5A}, C(O)N(R^{6A})₂, S(O)(NR^{d})(R^{4A}), S(O)(NH)(R^{4A}), P(O)(OR^{5A})₂, P(O)[N(R^{6A})₂]₂, B(OR^{5A})₂ and P(O)(OR^{5A})N(R^{6A})₂,
R² is selected from the group consisting of:
H, halo, cyano, OC(O)R^{4B}, NHC(O)R^{4B}, OR^{5B}, SR^{5B}, NHSO₂R^{4B}, OP(O)(OR^{5B})₂, C₁₋₆ alkyl optionally substituted with 1-2 R^{a}, and heteroaryl including from 5-10 ring atoms, wherein from 1-4 ring atoms are heteroatoms, each independently selected from the group consisting of N, N(H), N(R^{d}), O, and S(O)₀₋₂, and wherein one or more of the heteroaryl ring carbon atoms are optionally substituted with from 1-2 independently selected R^{e}; or

R¹ and R² taken together with the carbon atoms to which each is attached forms a ring including from 3-8 ring atoms, wherein the ring includes: (a) from 2-8 ring carbon atoms, each of which is substituted with from 1-2 substituents independently selected from H, C₁₋₃ alkyl, halo, hydroxy, and oxo; and (b) from 0-3 ring heteroatoms which are each independently selected from the group consisting of N, N(H), N(R^{d}), O, and S(O)₀₋₂;

each of R³, R⁴, and R⁵ is independently selected from the group consisting of:

(i) H, (ii) halo, (iii) C₁₋₆ alkyl which is optionally substituted with from 1-2 R^{a}, (iv) C₁₋₆ alkoxy which is optionally substituted with from 1-2 R^{a}, (v) C₁₋₆ haloalkoxy which is optionally substituted with from 1-2 R^{a}, (vi) —NR^{e}R^{f}, (vii) heteroaryl including from 5-10 ring atoms, wherein from 1-4 ring atoms are heteroatoms, each independently selected from the group consisting of N, N(H), N(R^{d}), O, and S(O)₀₋₂, and wherein one or more of the heteroaryl ring carbon atoms are optionally substituted with from 1-2 independently selected R^{e}, (viii) C₆₋₁₀ aryl, which is optionally substituted with from 1-2 R^{e}; or

R³ and R⁴ taken together with the carbon to which each is attached forms a ring including from 3-8 ring atoms, wherein the ring includes: (a) from 2-8 ring carbon atoms, each of which is substituted with from 1-2 substituents independently selected from H, C₁. 3 alkyl, halo, hydroxy, and oxo; and (b) from 0-3 ring heteroatoms which are each independently selected from the group consisting of N, N(H), N(R^{d}), O, and S(O)₀₋₂;

each of R^{4A}, R^{4B}, R^{5A} and R^{5B} is independently selected from the group consisting of:

### (i) H;

(ii) C₁₋₆ alkyl optionally substituted with 1-6 R^{a}; and
(iii) —(W′)_{q}—W², wherein:
q is 0 or 1;
W¹ is C₁₋₃ alkylene, which is optionally substituted with from 1-6 R^{a}; and
W² is:
   (a) C₃₋₁₀ cycloalkyl, which is optionally substituted with from 1-4 R^{b};
   (b) C₆₋₁₀ aryl, which is optionally substituted with from 1-4 R^{c};
   (c) heteroaryl including from 5-10 ring atoms, wherein from 1-4 ring atoms are heteroatoms, each independently selected from the group consisting of N, N(H), N(R^{d}), O, and S(O)₀₋₂, and wherein one or more of the heteroaryl ring carbon atoms are optionally substituted with from 1-4 independently selected R^{c}; or
   (d) heterocyclyl including from 3-10 ring atoms, wherein from 1-3 ring atoms are heteroatoms, each independently selected from the group consisting of N, N(H), N(R^{d}), O, and S(O)₀₋₂, and wherein one or more of the heterocyclyl ring carbon atoms are optionally substituted with from 1-4 independently selected R^{b};
      each occurrence of R^{6A} is independently:

### (i) H;

(ii) C₁₋₁₀ alkyl which is optionally substituted with 1-6 independently selected R^{a};
(iii) (C₀₋₃ alkylene)-C₃₋₁₀ cycloalkyl, which is optionally substituted with from 1-4 R^{b},
(iv) (C₀₋₃ alkylene)-C₆₋₁₀ aryl, which is optionally substituted with from 1-4 R^{c};
(v) (C₀₋₃ alkylene)-heteroaryl, wherein the heteroaryl includes from 5-10 ring atoms, wherein from 1-4 ring atoms are heteroatoms, each independently selected from the group consisting of N, N(H), N(R^{d}), O, and S(O)₀₋₂, and wherein one or more of the heteroaryl ring carbon atoms are optionally substituted with from 1-4 independently selected R^{c};
(vi) (C₀₋₃ alkylene)-heterocyclyl, wherein the heterocyclyl includes from 3-10 ring atoms, wherein from 1-3 ring atoms are heteroatoms, each independently selected from the group consisting of N, N(H), N(R^{d}), O, and S(O)₀₋₂, and wherein one or more of the heterocyclyl ring carbon atoms are optionally substituted with from 1-4 independently selected R^{b}; or
(vii) C₁₋₄ alkoxy; or

two occurrences of R^{6A} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R⁶), which are each independently selected from the group consisting of N(H), N(R^{d}), O, and S(O)₀₋₂;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;

each occurrence of R^{b} is independently selected from the group consisting of: C₁. alkyl optionally substituted with from 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₄ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; C₆₋₁₀ aryl optionally substituted with 1-4 independently selected C₁₋₄ alkyl; and C₃₋₆ cycloalkyl optionally substituted with from 1-4 independently selected C₁₋₄ alkyl;

each occurrence of R^{c} is independently selected from the group consisting of:

(i) halo; (ii) cyano; (iii) C₁₋₁₀ alkyl which is optionally substituted with from 1-6 independently selected R^{a}; (iv) C₂₋₆ alkenyl; (v) C₂₋₆ alkynyl; (vi) C₁₋₄ haloalkyl; (vii) C₁₋₄ alkoxy; (viii) C₁₋₄ haloalkoxy; (ix) —(C₀₋₃ alkylene)-C₃₋₆ cycloalkyl optionally substituted with from 1-4 independently selected C₁₋₄ alkyl; (x) —(C₀₋₃ alkylene)-C₆₋₁₀ aryl optionally substituted with from 1-4 independently selected C₁₋₄ alkyl; (xi) —(C₀₋₃ alkylene)-heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 independently selected C₁₋₄ alkyl; (xii) —S(O)₁₋₂(C₁₋₄ alkyl); (xiii) —NR′R^{f}; (xiv) —OH; (xv) —S(O)₁₋₂(NR′R″); (xvi) —C₁₋₄ thioalkoxy; (xvii) —NO₂; (xviii) —C(═O)(C₁₋₄ alkyl); (xix) —C(═O)O(C₁₋₄ alkyl); (xx) —C(═O)OH, and (xxi) —C(═O)N(R′)(R″);

R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R^{e} and R^{f}), which are each independently selected from the group consisting of N(H), N(R^{d}), O, and S(O)₀₋₂; and

each occurrence of R′ and R″ is independently selected from the group consisting of: H and C₁₋₄ alkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R′ and R″), which are each independently selected from the group consisting of N(H), N(R^{d}), O, and S(O)₀₋₂.

In another aspect, the STING antagonist is a compound selected from the group consisting of compounds in Table 7 and pharmaceutically acceptable salts thereof.

TABLE 7

| Compound | |
|---|---|
| # | Structure |
| 47 | 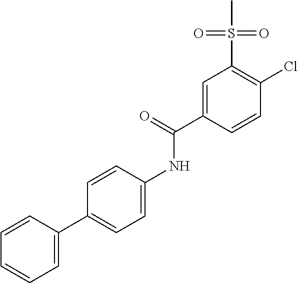 [SMILES: CS(=O)(=O)c1cc(C(=O)Nc2ccc(-c3ccccc3)cc2)ccc1Cl] |
| 48 | 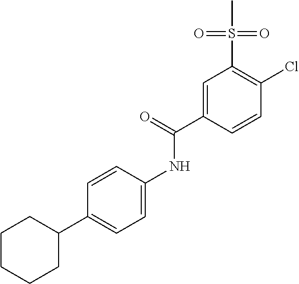 [SMILES: CS(=O)(=O)c1cc(C(=O)Nc2ccc(C3CCCCC3)cc2)ccc1Cl] |
| 49 | 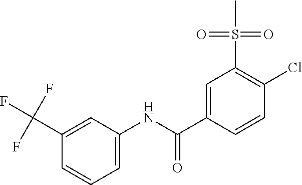 [SMILES: CS(=O)(=O)c1cc(C(=O)Nc2cccc(C(F)(F)F)c2)ccc1Cl] |
| 64 | 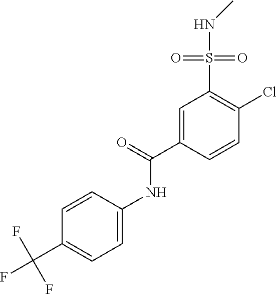 [SMILES: CNS(=O)(=O)c1cc(C(=O)Nc2ccc(C(F)(F)F)cc2)ccc1Cl] |

Compounds of Formula (VIII) and Table 7, and methods of making and using the same are further described in WO 2020/106741, filed as PCT/US2019/062245 on Nov. 19, 2019; U.S. Provisional 62/861,108, filed on Jun. 13, 2019; and U.S. Provisional 62/769,500, filed on Nov. 19, 2018, each of which is incorporated herein by reference in its entirety.

In another aspect, the STING antagonist is a compound of Formula (IX):

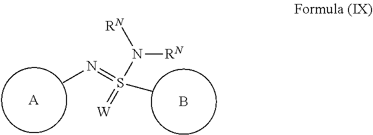

or a pharmaceutically acceptable salt thereof or a tautomer thereof, wherein:
A is selected from the group consisting of:
(i) heteroaryl including from 5-6 ring atoms, wherein from 1-4 ring atoms are heteroatoms, each independently selected from the group consisting of N, N(H), N(R¹), N(R²), O, S, and S(O)₂, and wherein from 1-5 ring atoms are carbon atoms, each independently selected from the group consisting of C, CH, CR¹, and CR³; provided that at least one ring atom is substituted with R¹; and
(ii) heteroaryl including from 7-20 ring atoms, wherein from 1-4 ring atoms are heteroatoms, each independently selected from the group consisting of N, N(H), N(R¹), N(R²), O, and S(O)₀₋₂, and wherein from 3-19 ring atoms are carbon atoms, each independently selected from the group consisting of C, CH, CH₂, CR¹, CHR¹, C(R¹)₂, CR³, CHR³, and C(R³)₂;
B and each occurrence of R^{N} are defined according to (A) and (B) below:

### A

### B is:

(a) C₁₋₁₅ alkyl which is optionally substituted with from 1-6 R^{a};
(b) C₃₋₂₀ cycloalkyl, which is optionally substituted with from 1-4 R^{b};
(c) phenyl substituted with from 1-4 R^{c};
(d) C₈₋₂₀ aryl optionally substituted with from 1-4 R^{c};
(e) heteroaryl including from 5-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; or
(f) heterocyclyl including from 3-16 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b};
each R^{N} is independently:

### (i) H,

(ii) C₁₋₆ alkyl optionally substituted with from 1-3 R^{a},
(iii) C₃₋₆ cycloalkyl, optionally substituted with from 1-3 R^{a},
(iv) —C(O)(C₁₋₄ alkyl), and
(v) —C(O)O(C₁₋₄ alkyl),

### B

B and one R^{N}, taken together with the atoms to which each is attached form a ring including from 5-20 ring atoms, wherein the ring includes: (a) from 0-4 ring heteroatoms each independently selected from N, N(H), N(R^{d}), O, and S(O)₀₋₂ (in addition to the heteroatoms in the

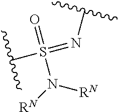

moiety); and (b) from 2 to 17 ring carbon atoms, each of which is optionally substituted with 1-2 substituents independently selected from

### (i) H;

(ii) oxo;
(iii) halo;
(iv) hydroxy;
(v) C₁₋₆ alkyl;
(vi) C₁₋₆ haloalkyl;
(vii) C₆₋₁₀ aryl optionally substituted with from 1-3 R^{c};
(viii) heteroaryl including from 5-20 ring atoms, wherein from 1-4 ring atoms are heteroatoms, each independently selected from the group consisting of N, N(H), N(R^{d}), O, and S(O)₀₋₂, and wherein the heteroaryl ring carbon atoms are optionally substituted with from 1-4 independently selected R^{c};
(ix) heterocyclyl including from 3-16 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b}; and
(x) C₃₋₂₀ cycloalkyl, which is optionally substituted with from 1-4 R^{b}; and
the remaining R^{N} is H or C₁₋₆ alkyl;

### W is O, NH, or N(R^{d});

### R is:

(i) —(U¹)_{q}—U², wherein:
q is 0 or 1;
U¹ is C₁₋₆ alkylene, which is optionally substituted with from 1-6 R^{a}; and
U² is:
   (a) C₃₋₁₂ cycloalkyl, which is optionally substituted with from 1-4 R^{b},
   (b) C₆₋₁₀ aryl, which is optionally substituted with from 1-4 R^{c};
   (c) heteroaryl including from 5-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}, or
   (d) heterocyclyl including from 3-12 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},

### OR

(ii) C₁₋₁₀ alkyl, which is optionally substituted with from 1-6 independently selected R^{a};
each occurrence of R² is independently selected from the group consisting of:
(i) C₁₋₆ alkyl, which is optionally substituted with from 1-4 independently selected R^{a};
(ii) C₃₋₆ cycloalkyl;
(iii) heterocyclyl including from 3-10 ring atoms, wherein from 1-3 ring atoms are heteroatoms, each independently selected from the group consisting of N, N(H), N(R^{d}), O, and S(O)₀₋₂;
(iv) —C(O)(C₁₋₄ alkyl);
(v) —C(O)O(C₁₋₄ alkyl);

### (vi) —CON(R′)(R″);

(vii) —S(O)₁₋₂(NR′R″);
(viii) —S(O)₁₋₂(C₁₋₄ alkyl);

### (ix) —OH; and

(x) C₁₋₄ alkoxy;
each occurrence of R³ is independently selected from the group consisting of halo, cyano, C₂₋₆ alkenyl, C₂₋₆ alkynyl, C₁₋₄ alkoxy, C₁₋₄ haloalkoxy, —S(O)₁₋₂(C₁₋₄ alkyl), —NR^{e}R^{f}, —OH, oxo, —S(O)₁₋₂(NR′R″), —C₁₋₄ thioalkoxy, —NO₂, —C(═O)(C₁₋₄ alkyl), —C(═O)O(C₁₋₄ alkyl), —C(═O)OH, and —C(═O)N(R′)(R″);

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;

each occurrence of R^{b} is independently selected from the group consisting of: C₁. alkyl optionally substituted with from 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₄ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of:

(a) halo; (b) cyano; (c) C₁₋₁₀ alkyl which is optionally substituted with from 1-6 independently selected R^{a}; (d) C₂₋₆ alkenyl; (e) C₂₋₆ alkynyl; (g) C₁₋₄ alkoxy; (h) C₁₋₄ haloalkoxy; (i) —S(O)₁₋₂(C₁₋₄ alkyl); (j) —NR^{e}R^{f}; (k) —OH; (l) —S(O)₁₋₂(NR′R″); (m) —C₁₋₄ thioalkoxy; (n) —NO₂; (o) —C(═O)(C₁₋₄ alkyl); (p) —C(═O)O(C₁₋₄ alkyl); (q) —C(═O)OH; (r) —C(═O)N(R′)(R″); and (s) -L¹-L²-R^{h};

R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene;
-L² is —O—, —N(H)—, —S—, or a bond;
R^{h} is selected from:
C₃₋₈ cycloalkyl optionally substituted with from 1-4 substituents independently selected from the group consisting of halo, C₁₋₄ alkyl, and C₁₋₄ haloalkyl (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo, C₁₋₄ alkyl, and C₁₋₄ haloalkyl;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo, C₁. 4 alkyl, and C₁₋₄ haloalkyl; and
C₆₋₁₀ aryl, which is optionally substituted with from 1-4 substituents independently selected from the group consisting of halo, C₁₋₄ alkyl, or C₁₋₄ haloalkyl; and
   each occurrence of R′ and R″ is independently selected from the group consisting of: H, C₁₋₄ alkyl, and C₆₋₁₀ aryl optionally substituted with from 1-2 substituents selected from halo, C₁₋₄ alkyl, and C₁₋₄ haloalkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(R^{d}), O, and S;

with the proviso that the compound is not:

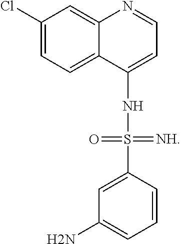 [SMILES: N=S(=O)(Nc1ccnc2cc(Cl)ccc12)c1cccc(N)c1]

In another aspect, the STING antagonist is a compound selected from the group consisting of compounds in Table 8 and pharmaceutically acceptable salts thereof.

TABLE 8

| Compound | |
|---|---|
| # | Structure |
| 1 | 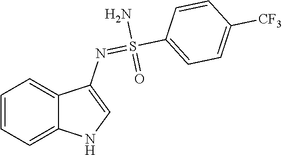 [SMILES: NS(=O)(=Nc1c[nH]c2ccccc12)c1ccc(C(F)(F)F)cc1] |
| 1a | 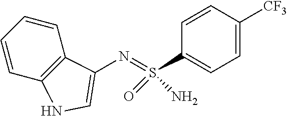 [SMILES: N[S@](=O)(=Nc1c[nH]c2ccccc12)c1ccc(C(F)(F)F)cc1] |
| 1b | 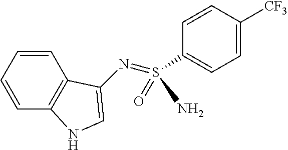 [SMILES: N[S@@](=O)(=Nc1c[nH]c2ccccc12)c1ccc(C(F)(F)F)cc1] |
| 2 | 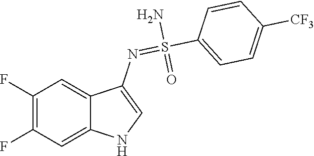 [SMILES: NS(=O)(=Nc1c[nH]c2cc(F)c(F)cc12)c1ccc(C(F)(F)F)cc1] |
| 2a | 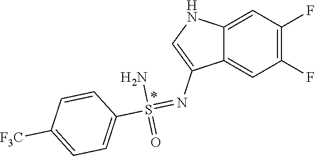 [SMILES: NS(=O)(=Nc1c[nH]c2cc(F)c(F)cc12)c1ccc(C(F)(F)F)cc1] |
| 2b | 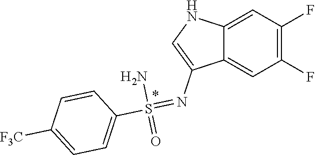 [SMILES: NS(=O)(=Nc1c[nH]c2cc(F)c(F)cc12)c1ccc(C(F)(F)F)cc1] |
| 109 | 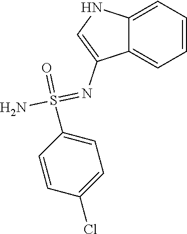 [SMILES: NS(=O)(=Nc1c[nH]c2ccccc12)c1ccc(Cl)cc1] |
| 109a | 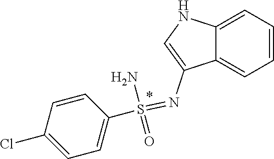 [SMILES: NS(=O)(=Nc1c[nH]c2ccccc12)c1ccc(Cl)cc1] |
| 109b | 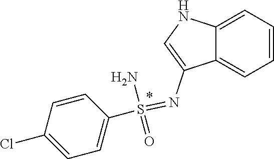 [SMILES: NS(=O)(=Nc1c[nH]c2ccccc12)c1ccc(Cl)cc1] |
| 110 | 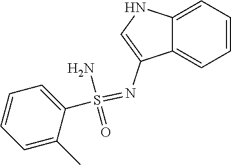 [SMILES: Cc1ccccc1S(N)(=O)=Nc1c[nH]c2ccccc12] |
| 110a | 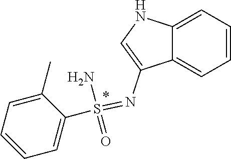 [SMILES: Cc1ccccc1S(N)(=O)=Nc1c[nH]c2ccccc12] |
| 110b | 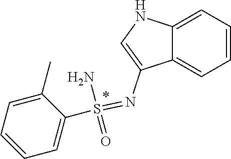 [SMILES: Cc1ccccc1S(N)(=O)=Nc1c[nH]c2ccccc12] |
| 111 | 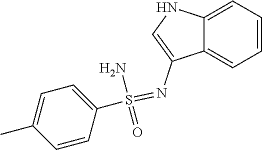 [SMILES: Cc1ccc(S(N)(=O)=Nc2c[nH]c3ccccc23)cc1] |
| 111a | 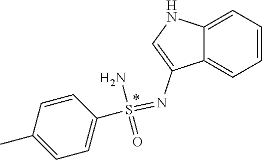 [SMILES: Cc1ccc(S(N)(=O)=Nc2c[nH]c3ccccc23)cc1] |
| 111b | 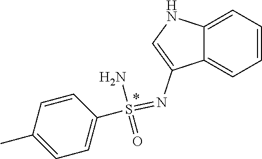 [SMILES: Cc1ccc(S(N)(=O)=Nc2c[nH]c3ccccc23)cc1] |
| 118 | 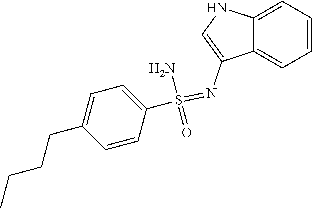 [SMILES: CCCCc1ccc(S(N)(=O)=Nc2c[nH]c3ccccc23)cc1] |
| 118a | 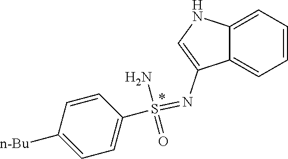 |
| 118b | 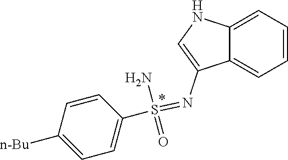 |

Compounds of Formula (IX) and Table 8, and methods of making and using the same are further described in WO 2020/106736, filed as PCT/US2019/062238 on Nov. 19, 2019; U.S. Provisional 62/769,327, filed on Nov. 19, 2018; and U.S. Provisional 62/861,781, filed on Jun. 14, 2019, each of which is incorporated herein by reference in its entirety.

In another aspect, the STING antagonist is a compound of Formula (X):

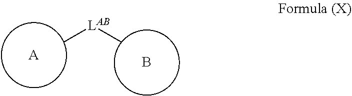

or a pharmaceutically acceptable salt thereof or a tautomer thereof, wherein:
L^{AB} is —N(R^{N})S(O)₂—* —N(R^{N})S(O)₂—(W^{AB1}-W^{AB2}—W^{AB3})—*, —S(O)₂N(R^{N})—*,
wherein the asterisk represents point of attachment to B;
W^{AB1} is C₁₋₃ alkylene optionally substituted with from 1-4 independently selected R^{a};
W^{AB2} is a bond, —O—, —NR^{N}, or —S—;
W^{AB3} is a bond or C₁₋₃ alkylene optionally substituted with from 1-4 independently selected R^{a};
A is selected from the group consisting of:
(i) heteroaryl including from 5-6 ring atoms, wherein from 1-4 ring atoms are heteroatoms, each independently selected from the group consisting of N, N(H), N(R¹), N(R²), O, and S, and wherein from 1-5 ring atoms are carbon atoms, each independently selected from the group consisting of C, CH, CR¹, and CR³; provided that at least one ring atom is substituted with R¹; and
(ii) heteroaryl including from 7-20 ring atoms, wherein from 1-4 ring atoms are heteroatoms, each independently selected from the group consisting of N, N(H), N(R¹), N(R²), O, and S(O)₀₋₂, and wherein from 3-19 ring atoms are carbon atoms, each independently selected from the group consisting of C, CH, CH₂, CR¹, CHR, C(R¹)₂, CR³, CHR³, and C(R³)₂;

### B is:

(a) C₁₋₁₅ alkyl which is optionally substituted with from 1-6 R^{a};
(b) C₃₋₂₀ cycloalkyl, which is optionally substituted with from 1-4 R^{b};
(c) C₆₋₂₀ aryl optionally substituted with from 1-4 R^{c};
(d) heteroaryl including from 5-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; or
(e) heterocyclyl including from 3-16 ring atoms, wherein from 1-3 ring atoms are heteroatoms, each independently selected from the group consisting of N(H), N(R^{d}), O, and S(O)₀₋₂ and wherein the heterocyclyl ring is optionally substituted with from 1-4 independently selected R^{b};
R^{N} is: (i) H, or (ii) C₁₋₆ alkyl optionally substituted with from 1-3 R^{a},

### R¹ is:

(i) —(U¹)_{q}—U², wherein:
q is 0 or 1;
U¹ is C₁₋₆ alkylene, which is optionally substituted with from 1-6 R^{a}; and
U² is:
   (a) C₃₋₁₂ cycloalkyl, which is optionally substituted with from 1-4 R^{b},
   (b) C₆₋₁₀ aryl, which is optionally substituted with from 1-4 R^{c};
   (c) heteroaryl including from 5-20 ring atoms, wherein from 1-4 ring atoms are heteroatoms, each independently selected from the group consisting of N, N(H), N(R^{d}), O, S(O)₀₋₂, and wherein the heteroaryl ring is optionally substituted with from 1-4 independently selected R^{c}, or
   (d) heterocyclyl including from 3-12 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},

### OR

(ii) C₁₋₁₀ alkyl, which is optionally substituted with from 1-6 independently selected R^{a};

each occurrence of R² is independently selected from the group consisting of:

(i) C₁₋₆ alkyl, which is optionally substituted with from 1-2 independently selected R^{a}; (ii) C₃₋₆ cycloalkyl; (iii) heterocyclyl including from 3-10 ring atoms, wherein from 1-3 ring atoms are heteroatoms, each independently selected from the group consisting of N, N(H), N(R^{d}), O, and S(O)₀₋₂; (iv) —C(O)(C₁₋₄ alkyl); (v) —C(O)O(C₁₋₄ alkyl); (vi) —CON(R′)(R″); (vii) —S(O)₁₋₂(NR′R″); (viii) —S(O)₁₋₂(C₁₋₄ alkyl); (ix) —OH; and (x) C₁₋₄ alkoxy;

each occurrence of R³ is independently selected from the group consisting of halo, cyano, C₂₋₆ alkenyl, C₂₋₆ alkynyl, C₁₋₄ alkoxy, C₁₋₄ haloalkoxy, —S(O)₁₋₂(C₁₋₄ alkyl), —NR^{e}R^{f}, —OH, oxo, —S(O)₁₋₂(NR′R″), —C₁₋₄ thioalkoxy, —NO₂, —C(═O)(C₁₋₄ alkyl), —C(═O)O(C₁₋₄ alkyl), —C(═O)OH, and —C(═O)N(R′)(R″);

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;

each occurrence of R^{b} is independently selected from the group consisting of: C₁. alkyl optionally substituted with from 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₄ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of:

(a) halo; (b) cyano; (c) C₁₋₁₅ alkyl which is optionally substituted with from 1-6 independently selected R^{a}; (d) C₂₋₆ alkenyl; (e) C₂₋₆ alkynyl; (g) C₁₋₄ alkoxy optionally substituted with from 1-3 independently selected R^{a}; (h) C₁₋₄ haloalkoxy; (i) —S(O)₁₋₂(C₁₋₄ alkyl); (j) —NR^{e}R^{f}; (k) —OH; (l) —S(O)₁₋₂(NR′R″); (m) —C₁₋₄ thioalkoxy; (n) —NO₂; (o) —C(═O)(C₁₋₄ alkyl); (p) —C(═O)O(C₁₋₄ alkyl); (q) —C(═O)OH; (r) —C(═O)N(R′)(R″); and (s) -L¹-L²-R^{h}.

R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R′ and R″), which are each independently selected from the group consisting of N(R^{d}), NH, O, and S;

-L¹ is a bond or C₁₋₃ alkylene;
-L² is —O—, —N(H)—, —S—, or a bond;
R^{h} is selected from:
C₃₋₈ cycloalkyl optionally substituted with from 1-4 substituents independently selected from the group consisting of halo, C₁₋₄ alkyl, and C₁₋₄ haloalkyl (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂ wherein the heterocyclyl is optionally substituted with from 1-4 substituents independently selected from the group consisting of halo, C₁₋₄ alkyl, and C₁₋₄ haloalkyl;
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 substituents independently selected from the group consisting of halo, C₁₋₄ alkyl, and C₁₋₄ haloalkyl; and
C₆₋₁₀ aryl, which is optionally substituted with from 1-4 substituents independently selected from the group consisting of halo, C₁₋₄ alkyl, or C₁₋₄ haloalkyl; and

each occurrence of R′ and R″ is independently selected from the group consisting of: H, C₁₋₄ alkyl, and C₆₋₁₀ aryl optionally substituted with from 1-2 substituents selected from halo, C₁₋₄ alkyl, and C₁₋₄ haloalkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(R^{d}), O, and S.

In another aspect, the STING antagonist is a compound selected from the group consisting of compounds in Table 9 and pharmaceutically acceptable salts thereof.

TABLE 9

| Com- | |
|---|---|
| pound | |
| # | Structure |
| 106 | 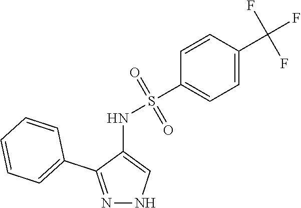 [SMILES: O=S(=O)(Nc1c[nH]nc1-c1ccccc1)c1ccc(C(F)(F)F)cc1] |
| 128 | 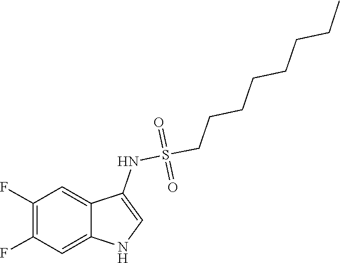 [SMILES: CCCCCCCCS(=O)(=O)Nc1c[nH]c2cc(F)c(F)cc12] |
| 131 | 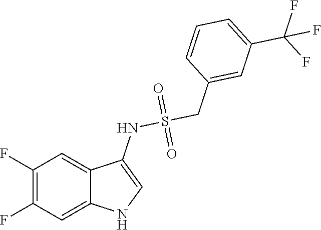 [SMILES: O=S(=O)(Cc1cccc(C(F)(F)F)c1)Nc1c[nH]c2cc(F)c(F)cc12] |
| 135 | 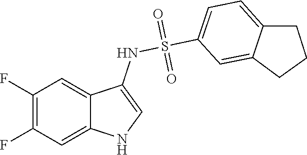 [SMILES: O=S(=O)(Nc1c[nH]c2cc(F)c(F)cc12)c1ccc2c(c1)CCC2] |
| 139 | 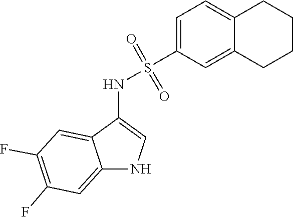 [SMILES: O=S(=O)(Nc1c[nH]c2cc(F)c(F)cc12)c1ccc2c(c1)CCCC2] |
| 141 | 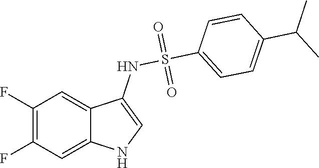 [SMILES: CC(C)c1ccc(S(=O)(=O)Nc2c[nH]c3cc(F)c(F)cc23)cc1] |

Compounds of Formula (X) and Table 9, and methods of making and using the same are further described in PCT/US2020/013824, filed on Jan. 16, 2020; U.S. Provisional 62/793,623, filed on Jan. 17, 2019; and U.S. Provisional 62/861,702, filed on Jun. 14, 2019, which is incorporated herein by reference in its entirety.

### STING Inhibitory Nucleic Acids

In some embodiments of any of the methods described herein, the STING antagonist is an inhibitory nucleic acid. In some embodiments, the inhibitory nucleic acid is a short interfering RNA, an antisense nucleic acid, a cyclic dinucleotide, or a ribozyme.

Examples of aspects of these different oligonucleotides are described below. Any of the examples of inhibitory nucleic acids that are STING antagonists can decrease expression of STING mRNA in a mammalian cell (e.g., a human cell). Any of the inhibitory nucleic acids described herein can be synthesized in vitro.

Inhibitory nucleic acids that can decrease the expression of STING mRNA expression in a mammalian cell include antisense nucleic acid molecules, i.e., nucleic acid molecules whose nucleotide sequence is complementary to all or part of a STING mRNA (e.g., complementary to all or a part of any one of SEQ ID NOs: 1, 3, 5, or 7).

An antisense nucleic acid molecule can be complementary to all or part of a non-coding region of the coding strand of a nucleotide sequence encoding a STING protein. Non-coding regions (5′ and 3′ untranslated regions) are the 5′ and 3′ sequences that flank the coding region in a gene and are not translated into amino acids.

Based upon the sequences disclosed herein, one of skill in the art can easily choose and synthesize any of a number of appropriate antisense nucleic acids to target a nucleic acid encoding a STING protein described herein. Antisense nucleic acids targeting a nucleic acid encoding a STING protein can be designed using the software available at the Integrated DNA Technologies website.

Examples of modified nucleotides which can be used to generate an antisense nucleic acid include 1-methylguanine, 1-methylinosine, 2,2-dimethylguanine, 2-methyladenine, 2-methylguanine, 3-methylcytosine, 2-methylthio-N6-isopentenyladenine, uracil-5-oxyacetic acid (v), wybutoxosine, pseudouracil, queosine, 2-thiocytosine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules described herein can be prepared in vitro and administered to a subject, e.g., a human subject. Alternatively, they can be generated in situ such that they hybridize with or bind to cellular mRNA and/or genomic DNA encoding a STING protein to thereby inhibit expression, e.g., by inhibiting transcription and/or translation. The hybridization can be by conventional nucleotide complementarities to form a stable duplex, or, for example, in the case of an antisense nucleic acid molecule that binds to DNA duplexes, through specific interactions in the major groove of the double helix. The antisense nucleic acid molecules can be delivered to a mammalian cell using a vector (e.g., an adenovirus vector, a lentivirus, or a retrovirus).

An antisense nucleic acid can be an α-anomeric nucleic acid molecule. An α-anomeric nucleic acid molecule forms specific double-stranded hybrids with complementary RNA in which, contrary to the usual, β-units, the strands run parallel to each other (Gaultier et al., Nucleic Acids Res. 15:6625-6641, 1987). The antisense nucleic acid can also comprise a chimeric RNA-DNA analog (Inoue et al., FEBS Lett. 215:327-330, 1987) or a 2′-O-methylribonucleotide (Inoue et al., Nucleic Acids Res. 15:6131-6148, 1987).

Another example of an inhibitory nucleic acid is a ribozyme that has specificity for a nucleic acid encoding a STING mRNA, e.g., specificity for any one of SEQ ID NOs: 1, 3, 5, or 7). Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature 334:585-591, 1988)) can be used to catalytically cleave mRNA transcripts to thereby inhibit translation of the protein encoded by the mRNA. STING mRNA can be used to select a catalytic RNA having a specific ribonuclease activity from a pool of RNA molecules. See, e.g., Bartel et al., Science 261:1411-1418, 1993.

Alternatively, a ribozyme having specificity for a STING mRNA sequence disclosed herein. For example, a derivative of a Tetrahymena L-19 IVS RNA can be constructed in which the nucleotide sequence of the active site is complementary to the nucleotide sequence to be cleaved in a STING mRNA (see, e.g., U.S. Pat. Nos. 4,987,071 and 5,116,742).

An inhibitory nucleic acid can also be a nucleic acid molecule that forms triple helical structures. For example, expression of a STING polypeptide can be inhibited by targeting nucleotide sequences complementary to the regulatory region of the gene encoding the STING polypeptide (e.g., the promoter and/or enhancer, e.g., a sequence that is at least 1 kb, 2 kb, 3 kb, 4 kb, or 5 kb upstream of the transcription initiation start state) to form triple helical structures that prevent transcription of the gene in target cells. See generally Maher, Bioassays 14(12):807-15, 1992; Helene, Anticancer Drug Des. 6(6):569-84, 1991; and Helene, Ann. N.Y. Acad. Sci. 660:27-36, 1992.

In various embodiments, inhibitory nucleic acids can be modified at the sugar moiety, the base moiety, or phosphate backbone to improve, e.g., the solubility, stability, or hybridization, of the molecule. For example, the deoxyribose phosphate backbone of the nucleic acids can be modified to generate peptide nucleic acids (see, e.g., Hyrup et al., Bioorganic Medicinal Chem. 4(1):5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to RNA and DNA under conditions of low ionic strength. PNA oligomers can be synthesized using standard solid phase peptide synthesis protocols (see, e.g., Perry-O'Keefe et al., Proc. Natl. Acad. Sci. U.S.A. 93:14670-675, 1996). PNAs can be used as antisense or antigene agents for sequence-specific modulation of gene expression by, e.g., inducing transcription or translation arrest or inhibiting replication.

cGAS Inhibitors

In any of the methods described herein, the cGAS inhibitors can be any of the cGAS inhibitors described herein (e.g., any of the compounds described in this section). In any of the methods described herein, the cGAS inhibitor has an IC₅₀ of between about 1 nM and about 10 μM for cGAS.

In one aspect, the cGAS inhibitor is a compound selected from the group consisting of compounds in Table 10 and pharmaceutically acceptable salts thereof.

TABLE 10

| Example | |
|---|---|
| # | Structure |
| 301 | 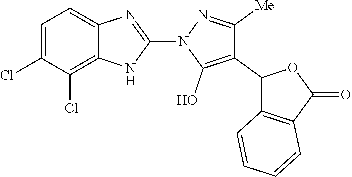 [SMILES: Cc1nn(-c2nc3ccc(Cl)c(Cl)c3[nH]2)c(O)c1C1OC(=O)c2ccccc21] |
| 302 | 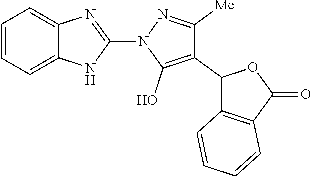 [SMILES: Cc1nn(-c2nc3ccccc3[nH]2)c(O)c1C1OC(=O)c2ccccc21] |
| 303 | 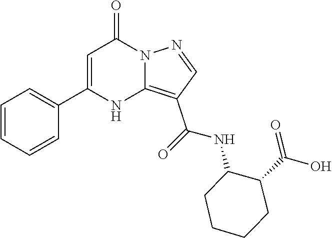 |
| 304 | 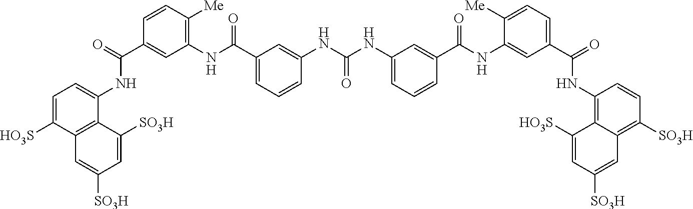 [SMILES: Cc1ccc(C(=O)Nc2ccc(S(=O)(=O)O)c3cc(S(=O)(=O)O)cc(S(=O)(=O)O)c23)cc1NC(=O)c1cccc(NC(=O)Nc2cccc(C(=O)Nc3cc(C(=O)Nc4ccc(S(=O)(=O)O)c5cc(S(=O)(=O)O)cc(S(=O)(=O)O)c45)ccc3C)c2)c1] . |

In some embodiments, the cGAS inhibitor is selected from the compounds disclosed in U.S. Provisional 62/355,403, filed on Jun. 28, 2016, which is incorporated herein by reference in its entirety.

In some embodiments, the cGAS inhibitor is selected from the compounds disclosed in U.S. Provisional 62/318,435, filed on Apr. 5, 2016, which is incorporated herein by reference in its entirety.

In some embodiments, the cGAS inhibitor is selected from the compounds disclosed in US Application 2018/0230115 A1, published Aug. 16, 2018, which is incorporated herein by reference in its entirety.

In some embodiments, the cGAS inhibitor is selected from the compounds disclosed in Vincent, J. et al. (2017) Nat. Commun. 8(1):750, which is incorporated herein by reference in its entirety.

In some embodiments, the cGAS inhibitor is selected from the compounds disclosed in Hall, J. et al. (2017) PLOS ONE 12(9):e184843, which is incorporated herein by reference in its entirety.

In some embodiments, the cGAS inhibitor is selected from the compounds disclosed in Wang, M. et al. (2018) Future Med. Chem. 10(11):1301-17, which is incorporated herein by reference in its entirety.

In some embodiments, the cGAS inhibitor is selected from the compounds disclosed in U.S. Provisional 62/559,482, filed on Sep. 15, 2017, which is incorporated herein by reference in its entirety.

In some embodiments, the cGAS inhibitor is selected from the compounds disclosed in U.S. Provisional 62/633,248, filed on Feb. 21, 2018, which is incorporated herein by reference in its entirety.

In some embodiments, the cGAS inhibitor is selected from the compounds disclosed in U.S. Provisional 62/687,769, filed on Jun. 20, 2018, which is incorporated herein by reference in its entirety.

### Pharmaceutical Compositions

In some embodiments, an STING antagonist or cGAS inhibitor (e.g., any of the STING antagonists or cGAS inhibitors described herein or known in the art) is administered as a pharmaceutical composition that includes the chemical entity and one or more pharmaceutically acceptable excipients, and optionally one or more additional therapeutic agents as described herein.

In some embodiments, the STING antagonist or cGAS inhibitor can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of the STING antagonists or cGAS inhibitors described herein. Dosage forms or compositions containing an STING antagonist or cGAS inhibitor as described herein in the range of 0.005% to 100% with the balance made up from non-toxic excipient may be prepared. The contemplated compositions may contain 0.001%-100% of a STING antagonist, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, the STING antagonist or cGAS inhibitor (e.g., any of the exemplary STING antagonists or cGAS inhibitors described herein or known in the art) or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases, the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the STING antagonist or cGAS inhibitor in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

In certain embodiments, the STING antagonist or cGAS inhibitor or a pharmaceutical composition thereof are suitable for local, topical administration to the digestive or GI tract, e.g., rectal administration. Rectal compositions include, without limitation, enemas, rectal gels, rectal foams, rectal aerosols, suppositories, jelly suppositories, and enemas (e.g., retention enemas).

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the STING antagonist or cGAS inhibitor with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the STING antagonist or cGAS inhibitor or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the STING antagonist or cGAS inhibitor is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a STING antagonist or cGAS inhibitor, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more STING antagonists or cGAS inhibitors or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments, the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the STING antagonist or cGAS inhibitor to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the STING antagonist or cGAS inhibitor are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

### Enema Formulations

In some embodiments, enema formulations containing an STING antagonist or cGAS inhibitor are provided in “ready-to-use” form.

In some embodiments, enema formulations containing an STING antagonist or cGAS inhibitor are provided in one or more kits or packs. In certain embodiments, the kit or pack includes two or more separately contained/packaged components, e.g. two components, which when mixed together, provide the desired formulation (e.g., as a suspension). In certain of these embodiments, the two component system includes a first component and a second component, in which: (i) the first component (e.g., contained in a sachet) includes the STING antagonist or cGAS inhibitor (as described anywhere herein) and optionally one or more pharmaceutically acceptable excipients (e.g., together formulated as a solid preparation, e.g., together formulated as a wet granulated solid preparation); and (ii) the second component (e.g., contained in a vial or bottle) includes one or more liquids and optionally one or more other pharmaceutically acceptable excipients together forming a liquid carrier. Prior to use (e.g., immediately prior to use), the contents of (i) and (ii) are combined to form the desired enema formulation, e.g., as a suspension. In other embodiments, each of component (i) and (ii) is provided in its own separate kit or pack.

In some embodiments, each of the one or more liquids is water, or a physiologically acceptable solvent, or a mixture of water and one or more physiologically acceptable solvents. Typical such solvents include, without limitation, glycerol, ethylene glycol, propylene glycol, polyethylene glycol and polypropylene glycol. In certain embodiments, each of the one or more liquids is water. In other embodiments, each of the one or more liquids is an oil, e.g. natural and/or synthetic oils that are commonly used in pharmaceutical preparations.

Further pharmaceutical excipients and carriers that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe für Pharmazie, Kosmetik and angrenzende Gebiete (Edition Cantor Aulendorf, 1989)).

In some embodiments, each of the one or more pharmaceutically acceptable excipients can be independently selelcted from thickeners, viscosity enhancing agents, bulking agents, mucoadhesive agents, penetration enhanceers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents.

In certain embodiments, each of the one or more pharmaceutically acceptable excipients can be independently selected from thickeners, viscosity enhancing agents, mucoadhesive agents, buffers, preservatives, diluents, binders, lubricants, glidants, disintegrants, and fillers.

In certain embodiments, each of the one or more pharmaceutically acceptable excipients can be independently selected from thickeners, viscosity enhancing agents, bulking agents, mucoadhesive agents, buffers, preservatives, and fillers.

In certain embodiments, each of the one or more pharmaceutically acceptable excipients can be independently selected from diluents, binders, lubricants, glidants, and disintegrants.

Examples of thickeners, viscosity enhancing agents, and mucoadhesive agents include without limitation: gums, e.g. xanthan gum, guar gum, locust bean gum, tragacanth gums, karaya gum, ghatti gum, cholla gum, psyllium seed gum and gum arabic; poly(carboxylic acid-containing) based polymers, such as poly (acrylic, maleic, itaconic, citraconic, hydroxyethyl methacrylic or methacrylic) acid which have strong hydrogen-bonding groups, or derivatives thereof such as salts and esters; cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof, clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminium oxide; atherocollagen; polyvinyl polymers such as carboxyvinyl polymers; polyvinylpyrrolidone (povidone); polyvinyl alcohol; polyvinyl acetates, polyvinylmethyl ethers, polyvinyl chlorides, polyvinylidenes, and/or the like; polycarboxylated vinyl polymers such as polyacrylic acid as mentioned above; polysiloxanes; polyethers; polyethylene oxides and glycols; polyalkoxys and polyacrylamides and derivatives and salts thereof. Preferred examples can include cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof (e.g., methyl cellulose); and polyvinyl polymers such as polyvinylpyrrolidone (povidone).

Examples of preservatives include without limitation: benzalkonium chloride, benzoxonium chloride, benzethonium chloride, cetrimide, sepazonium chloride, cetylpyridinium chloride, domiphen bromide (Bradosol®), thiomersal, phenylmercuric nitrate, phenylmercuric acetate, phenylmercuric borate, methylparaben, propylparaben, chlorobutanol, benzyl alcohol, phenyl ethyl alcohol, chlorohexidine, polyhexamethylene biguanide, sodium perborate, imidazolidinyl urea, sorbic acid, Purite®), Polyquart®), and sodium perborate tetrahydrate and the like.

In certain embodiments, the preservative is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C1-C4 alkyl. In certain embodiments, the preservative is methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof.

Examples of buffers include without limitation: phosphate buffer system (sodium dihydrogen phosphate dehydrate, disodium phosphate dodecahydrate, bibasic sodium phosphate, anhydrous monobasic sodium phosphate), bicarbonate buffer system, and bisulfate buffer system.

Examples of disintegrants include, without limitation: carmellose calcium, low substituted hydroxypropyl cellulose (L-HPC), carmellose, croscarmellose sodium, partially pregelatinized starch, dry starch, carboxymethyl starch sodium, crospovidone, polysorbate 80 (polyoxyethylenesorbitan oleate), starch, sodium starch glycolate, hydroxypropyl cellulose pregelatinized starch, clays, cellulose, alginine, gums or cross linked polymers, such as cross-linked PVP (Polyplasdone XL from GAF Chemical Corp). In certain embodiments, the disintegrant is crospovidone.

Examples of glidants and lubricants (aggregation inhibitors) include without limitation: talc, magnesium stearate, calcium stearate, colloidal silica, stearic acid, aqueous silicon dioxide, synthetic magnesium silicate, fine granulated silicon oxide, starch, sodium laurylsulfate, boric acid, magnesium oxide, waxes, hydrogenated oil, polyethylene glycol, sodium benzoate, stearic acid glycerol behenate, polyethylene glycol, and mineral oil. In certain embodiments, the glidant/lubricant is magnesium stearate, talc, and/or colloidal silica; e.g., magnesium stearate and/or talc.

Examples of diluents, also referred to as “fillers” or “bulking agents” include without limitation: dicalcium phosphate dihydrate, calcium sulfate, lactose (e.g., lactose monohydrate), sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. In certain embodiments, the diluent is lactose (e.g., lactose monohydrate).

Examples of binders include without limitation: starch, pregelatinized starch, gelatin, sugars (including sucrose, glucose, dxtrose, lactose and sorbitol), polyethylene glycol, waxes, natural and synthetic gums such as acacia tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (povidone). In certain embodiments, the binder is polyvinylpyrrolidone (povidone).

In some embodiments, enema formulations containing a STING antagonist or cGAS inhibitor include water and one or more (e.g., all) of the following excipients:

One or more (e.g., one, two, or three) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);

One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof,

One or more (e.g., one or two; e.g., two) buffers, such as phosphate buffer system (e.g., sodium dihydrogen phosphate dehydrate, disodium phosphate dodecahydrate);

One or more (e.g., one or two, e.g., two) glidants and/or lubricants, such as magnesium stearate and/or talc;

One or more (e.g., one or two; e.g., one) disintegrants, such as crospovidone; and

One or more (e.g., one or two; e.g., one) diluents, such as lactose (e.g., lactose monohydrate).

In certain of these embodiments, the STING antagonist is a compound of any one of Formulas I-X or a compound shown in any one of Tables 1-10, or a pharmaceutically acceptable salt and/or hydrate and/or cocrystal thereof.

In certain embodiments, enema formulations containing an STING antagonist or cGAS inhibitor include water, methyl cellulose, povidone, methylparaben, propylparaben, sodium dihydrogen phosphate dehydrate, disodium phosphate dodecahydrate, crospovidone, lactose monohydrate, magnesium stearate, and talc. In certain of these embodiments, the STING antagonist is a compound of any one of Formulas I-X or a compound shown in any one of Tables 1-10, or a pharmaceutically acceptable salt and/or hydrate and/or cocrystal thereof.

In certain embodiments, enema formulations containing an STING antagonist or cGAS inhibitor are provided in one or more kits or packs. In certain embodiments, the kit or pack includes two separately contained/packaged components, which when mixed together, provide the desired formulation (e.g., as a suspension). In certain of these embodiments, the two component system includes a first component and a second component, in which: (i) the first component (e.g., contained in a sachet) includes the STING antagonist or cGAS inhibitor (as described anywhere herein) and one or more pharmaceutically acceptable excipients (e.g., together formulated as a solid preparation, e.g., together formulated as a wet granulated solid preparation); and (ii) the second component (e.g., contained in a vial or bottle) includes one or more liquids and one or more one or more other pharmaceutically acceptable excipients together forming a liquid carrier. In other embodiments, each of component (i) and (ii) is provided in its own separate kit or pack.

In certain of these embodiments, component (i) includes the STING antagonist or cGAS inhibitor (e.g., a compound of any one of Formulas I-X or a compound shown in any one of Tables 1-10, or a pharmaceutically acceptable salt and/or hydrate and/or cocrystal thereof) and one or more (e.g., all) of the following excipients:

(a) One or more (e.g., one) binders (e.g., a polyvinyl polymer, such as polyvinylpyrrolidone (povidone);

(b) One or more (e.g., one or two, e.g., two) glidants and/or lubricants, such as magnesium stearate and/or talc;

(c) One or more (e.g., one or two; e.g., one) disintegrants, such as crospovidone; and

(d) One or more (e.g., one or two; e.g., one) diluents, such as lactose (e.g., lactose monohydrate).

In certain embodiments, component (i) includes from about 40 weight percent to about 80 weight percent (e.g., from about 50 weight percent to about 70 weight percent, from about 55 weight percent to about 70 weight percent; from about 60 weight percent to about 65 weight percent; e.g., about 62.1 weight percent) of the STING antagonist or cGAS inhibitor (e.g., a compound of any one of Formulas I-X or a compound shown in any one of Tables 1-10, or a pharmaceutically acceptable salt and/or hydrate and/or cocrystal thereof).

In certain embodiments, component (i) includes from about 0.5 weight percent to about 5 weight percent (e.g., from about 1.5 weight percent to about 4.5 weight percent, from about 2 weight percent to about 3.5 weight percent; e.g., about 2.76 weight percent) of the binder (e.g., povidone).

In certain embodiments, component (i) includes from about 0.5 weight percent to about 5 weight percent (e.g., from about 0.5 weight percent to about 3 weight percent, from about 1 weight percent to about 3 weight percent; about 2 weight percent e.g., about 1.9 weight percent) of the disintegrant (e.g., crospovidone).

In certain embodiments, component (i) includes from about 10 weight percent to about 50 weight percent (e.g., from about 20 weight percent to about 40 weight percent, from about 25 weight percent to about 35 weight percent; e.g., about 31.03 weight percent) of the diluent (e.g., lactose, e.g., lactose monohydrate).

In certain embodiments, component (i) includes from about 0.05 weight percent to about 5 weight percent (e.g., from about 0.05 weight percent to about 3 weight percent) of the glidants and/or lubricants.

In certain embodiments (e.g., when component (i) includes one or more lubricants, such as magnesium stearate), component (i) includes from about 0.05 weight percent to about 1 weight percent (e.g., from about 0.05 weight percent to about 1 weight percent; from about 0.1 weight percent to about 1 weight percent; from about 0.1 weight percent to about 0.5 weight percent; e.g., about 0.27 weight percent) of the lubricant (e.g., magnesium stearate).

In certain embodiments (when component (i) includes one or more lubricants, such as talc), component (i) includes from about 0.5 weight percent to about 5 weight percent (e.g., from about 0.5 weight percent to about 3 weight percent, from about 1 weight percent to about 3 weight percent; from about 1.5 weight percent to about 2.5 weight percent; from about 1.8 weight percent to about 2.2 weight percent; about 1.93 weight percent) of the lubricant (e.g., talc).

In certain of these embodiments, each of (a), (b), (c), and (d) above is present.

In certain embodiments, component (i) includes the ingredients and amounts as shown in Table A.

TABLE A

| Ingredient | Weight Percent |
|---|---|
| A compound of any one of | 40 weight percent to about 80 weight |
| Formulas I-X or a compound | percent (e.g., from about 50 weight |
| shown in any one of Tables | percent to about 70 weight percent, |
| 1-10 | from about 55 weight percent to about |
| | 70 weight percent; from about 60 weight |
| | percent to about 65 weight percent; |
| | e.g., about 62.1 weight percent) |
| Crospovidone (Kollidon | 0.5 weight percent to about 5 weight |
| CL) | percent (e.g., from about 0.5 weight |
| | percent to about 3 weight percent, |
| | from about 1 weight percent to about |
| | 3 weight percent; about 1.93 weight |
| | percent |
| lactose monohydrate | about 10 weight percent to about 50 |
| (Pharmatose 200M) | weight percent (e.g., from about 20 |
| | weight percent to about 40 weight |
| | percent, from about 25 weight percent |
| | to about 35 weight percent; e.g., |
| | about 31.03 weight percent |
| Povidone (Kollidon | about 0.5 weight percent to about 5 |
| K30) | weight percent (e.g., from about 1.5 |
| | weight percent to about 4.5 weight |
| | percent, from about 2 weight percent |
| | to about 3.5 weight percent; e.g., |
| | about 2.76 weight percent |
| Talc | 0.5 weight percent to about 5 weight |
| | percent (e.g., from about 0.5 weight |
| | percent to about 3 weight percent, |
| | from about 1 weight percent to about |
| | 3 weight percent; from about 1.5 weight |
| | percent to about 2.5 weight percent; |
| | from about 1.8 weight percent to about |
| | 2.2 weight percent; e.g., about 1.93 |
| | weight percent |
| Magnesium stearate | about 0.05 weight percent to about 1 |
| | weight percent (e.g., from about 0.05 |
| | weight percent to about 1 weight percent; |
| | from about 0.1 weight percent to about |
| | 1 weight percent; from about 0.1 weight |
| | percent to about 0.5 weight percent; |
| | e.g., about 0.27 weight percent |

In certain embodiments, component (i) includes the ingredients and amounts as shown in Table B.

TABLE B

| Ingredient | Weight Percent |
|---|---|
| A compound of any one of Formulas | About 62.1 weight percent) |
| I-X or a compound shown in any | |
| one of Tables 1-10 | |
| Crospovidone (Kollidon CL) | About 1.93 weight percent |
| lactose monohydrate (Pharmatose | About 31.03 weight percent |
| 200M) | |
| Povidone (Kollidon K30) | About 2.76 weight percent |
| talc | About 1.93 weight percent |
| Magnesium stearate | About 0.27 weight percent |

In certain embodiments, component (i) is formulated as a wet granulated solid preparation. In certain of these embodiments an internal phase of ingredients (the STING antagonist or cGAS inhibitor, disintegrant, and diluent) are combined and mixed in a high-shear granulator. A binder (e.g., povidone) is dissolved in water to form a granulating solution. This solution is added to the Inner Phase mixture resulting in the development of granules. While not wishing to be bound by theory, granule development is believed to be facilitated by the interaction of the polymeric binder with the materials of the internal phase. Once the granulation is formed and dried, an external phase (e.g., one or more lubricants—not an intrinsic component of the dried granulation), is added to the dry granulation. It is believed that lubrication of the granulation is important to the flowability of the granulation, in particular for packaging.

In certain of the foregoing embodiments, component (ii) includes water and one or more (e.g., all) of the following excipients:

(a′) One or more (e.g., one, two; e.g., two) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);

(b′) One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof; and

(c′) One or more (e.g., one or two; e.g., two) buffers, such as phosphate buffer system (e.g., sodium dihydrogen phosphate dihydrate, disodium phosphate dodecahydrate);

In certain of the foregoing embodiments, component (ii) includes water and one or more (e.g., all) of the following excipients:

(a″) a first thickener, viscosity enhancing agent, binder, and/or mucoadhesive agent (e.g., a cellulose or cellulose ester or ether or derivative or salt thereof (e.g., methyl cellulose));

(a′″) a second thickener, viscosity enhancing agent, binder, and/or mucoadhesive agent (e.g., a polyvinyl polymer, such as polyvinylpyrrolidone (povidone));

(b″) a first preservative, such as a paraben, e.g., propyl 4-hydroxybenzoate (propylparaben), or a pharmaceutically acceptable salt or ester thereof;

(b″) a second preservative, such as a paraben, e.g., methyl 4-hydroxybenzoate (methylparaben), or a pharmaceutically acceptable salt or ester thereof,

(c″) a first buffer, such as phosphate buffer system (e.g., disodium phosphate dodecahydrate);

(c′″) a second buffer, such as phosphate buffer system (e.g., sodium dihydrogen phosphate dehydrate),

In certain embodiments, component (ii) includes from about 0.05 weight percent to about 5 weight percent (e.g., from about 0.05 weight percent to about 3 weight percent, from about 0.1 weight percent to about 3 weight percent; e.g., about 1.4 weight percent) of (a″).

In certain embodiments, component (ii) includes from about 0.05 weight percent to about 5 weight percent (e.g., from about 0.05 weight percent to about 3 weight percent, from about 0.1 weight percent to about 2 weight percent; e.g., about 1.0 weight percent) of (a′″).

In certain embodiments, component (ii) includes from about 0.005 weight percent to about 0.1 weight percent (e.g., from about 0.005 weight percent to about 0.05 weight percent; e.g., about 0.02 weight percent) of (b″).

In certain embodiments, component (ii) includes from about 0.05 weight percent to about 1 weight percent (e.g., from about 0.05 weight percent to about 0.5 weight percent; e.g., about 0.20 weight percent) of (b′″).

In certain embodiments, component (ii) includes from about 0.05 weight percent to about 1 weight percent (e.g., from about 0.05 weight percent to about 0.5 weight percent; e.g., about 0.15 weight percent) of (c″).

In certain embodiments, component (ii) includes from about 0.005 weight percent to about 0.5 weight percent (e.g., from about 0.005 weight percent to about 0.3 weight percent; e.g., about 0.15 weight percent) of (c′″).

In certain of these embodiments, each of (a″)-(c′″) is present.

In certain embodiments, component (ii) includes water (up to 100%) and the ingredients and amounts as shown in Table C.

TABLE C

| Ingredient | Weight Percent |
|---|---|
| methyl cellulose | 0.05 weight percent to about 5 weight |
| (Methocel A15C premium) | percent (e.g., from about 0.05 weight |
| | percent to about 3 weight percent, |
| | from about 0.1 weight percent to |
| | about 3 weight percent; e.g., about |
| | 1.4 weight percent |
| Povidone (Kollidon K30) | 0.05 weight percent to about 5 weight |
| | percent (e.g., from about 0.05 weight |
| | percent to about 3 weight percent, |
| | from about 0.1 weight percent to |
| | about 2 weight percent; e.g., about |
| | 1.0 weight percent |
| propyl 4-hydroxybenzoate | about 0.005 weight percent to about 0.1 |
| | weight percent (e.g., from about 0.005 |
| | weight percent to about 0.05 weight |
| | percent; e.g., about 0.02 weight percent) |
| methyl 4-hydroxybenzoate | about 0.05 weight percent to about 1 |
| | weight percent (e.g., from about 0.05 |
| | weight percent to about 0.5 weight |
| | percent; e.g., about 0.20 weight |
| | percent) |
| disodium phosphate | about 0.05 weight percent to about 1 |
| dodecahydrate | weight percent (e.g., from about 0.05 |
| | weight percent to about 0.5 weight |
| | percent; e.g., about 0.15 weight |
| | percent) |
| sodium dihydrogen | about 0.005 weight percent to about 0.5 |
| phospahate dihydrate | weight percent (e.g., from about 0.005 |
| | weight percent to about 0.3 weight |
| | percent; e.g., about 0.15 weight |
| | percent) |

In certain embodiments, component (ii) includes water (up to 1000%) and the ingredients and amounts as shown in Table D.

TABLE D

| Ingredient | Weight Percent |
|---|---|
| methyl cellulose (Methocel | about 1.4 weight percent |
| A15C premium) | |
| Povidone (Kollidon K30) | about 1.0 weight percent |
| propyl 4-hydroxybenzoate | about 0.02 weight percent |
| methyl 4-hydroxybenzoate | about 0.20 weight percent |
| disodium phosphate dodecahydrate | about 0.15 weight percent |
| sodium dihydrogen phospahate | about 0.15 weight percent |
| dihydrate | |

“Ready-to-use” enemas are generally be provided in a “single-use” sealed disposable container of plastic or glass. Those formed of a polymeric material preferably have sufficient flexibility for ease of use by an unassisted patient. Typical plastic containers can be made of polyethylene. These containers may comprise a tip for direct introduction into the rectum. Such containers may also comprise a tube between the container and the tip. The tip is preferably provided with a protective shield that is removed before use. Optionally the tip has a lubricant to improve patient compliance.

In some embodiments, the enema formulation (e.g., suspension) is poured into a bottle for delivery after it has been prepared in a separate container. In certain embodiments, the bottle is a plastic bottle (e.g., flexible to allow for delivery by squeezing the bottle), which can be a polyethylene bottle (e.g., white in color). In some embodiments, the bottle is a single chamber bottle, which contains the suspension or solution. In other embodiments, the bottle is a multichamber bottle, where each chamber contains a separate mixture or solution. In still other embodiments, the bottle can further include a tip or rectal cannula for direct introduction into the rectum. In some embodiments, the enema formulation can be delivered in the device that includes a plastic bottle, a breakable capsule, and a rectal cannula and single flow pack.

### Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the STING antagonist or cGAS inhibitor is administered at a dosage of from about 0.001 mg/kg to about 500 mg/kg.

In some embodiments, enema formulations include from about 0.5 mg to about 2500 mg of the chemical entity in from about 1 mL to about 3000 mL of liquid carrier.

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of an STING antagonist or cGAS inhibitor is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a STING antagonist or cGAS inhibitor is administered to an individual for a period of time followed by a separate period of time. In another embodiment, a STING antagonist or cGAS inhibitor is administered for a first period and a second period following the first period, with administration stopped during the second period, followed by a third period where administration of the STING antagonist or cGAS inhibitor is started and then a fourth period following the third period where administration is stopped. In an aspect of this embodiment, the period of administration of an STING antagonist or cGAS inhibitor followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Kits

Also provided herein are kits containing one or more (e.g., at least 1, 2, 3, 4, 5, 6, 7, 8, 9, 10, 11, 12, 14, 15, 16, 18, or 20) of any of the pharmaceutical compositions described herein. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein. The kits described herein are not so limited; other variations will be apparent to one of ordinary skill in the art.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Numbered Clauses

The compounds, compositions, methods, and other subject matter described herein are further described in the following numbered clauses:

1. A method of treating a subject in need thereof, the method comprising:

(a) identifying a subject having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level; and

(b) administering a treatment comprising a therapeutically effective amount of a STING antagonist or a cGAS inhibitor, or a pharmaceutically acceptable salt, solvate, or co-crystal thereof to the identified subject.

2. A method of treating a subject in need thereof, the method comprising administering a treatment comprising a therapeutically effective amount of a STING antagonist or a cGAS inhibitor, or a pharmaceutically acceptable salt, solvate, or co-crystal thereof to a subject identified as having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level.

3. A method of selecting a treatment for a subject in need thereof, the method comprising:

(a) identifying a subject having a cancer cell having (i) one or both of (i) decreased TREX1 level and/or activity, and (ii) increased cGAS/STING signaling pathway activity and/or (ii) an elevated level of cGAMP in a serum or tumor sample of the subject as compared to a reference level; and

(b) selecting for the identified subject a treatment comprising a therapeutically effective amount of a STING antagonist or a cGAS inhibitor, or a pharmaceutically acceptable salt, solvate, or co-crystal thereof.

4. A method of selecting a treatment for a subject in need thereof, the method comprising selecting a treatment comprising a therapeutically effective amount of a STING antagonist or cGAS inhibitor, or a pharmaceutically acceptable salt, solvate, or co-crystal thereof for a subject identified as having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level.

5. A method of selecting a subject for treatment, the method comprising:

(a) identifying a subject having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level; and

(b) selecting the identified subject for treatment with a therapeutically effective amount of a STING antagonist or a cGAS inhibitor, or a pharmaceutically acceptable salt, solvate, or co-crystal thereof.

6. A method of selecting a subject for participation in a clinical trial, the method comprising:

(a) identifying a subject having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level; and

(b) selecting the identified subject for participation in a clinical trial that comprises administration of a treatment comprising a therapeutically effective amount of a STING antagonist or a cGAS inhibitor, or a pharmaceutically acceptable salt, solvate, or co-crystal thereof.

7. A method of selecting a subject for participation in a clinical trial, the method comprising selecting a subject identified as having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level, for participation in a clinical trial that comprises administration of a treatment comprising a therapeutically effective amount of a STING antagonist or a cGAS inhibitor, or a pharmaceutically acceptable salt, solvate, or co-crystal thereof.

8. A method of predicting a subject's responsiveness to a STING antagonist or cGAS inhibitor, the method comprising:

(a) determining that a subject has a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level; and

(b) identifying that the subject determined to have (i) one or both of (i) decreased TREX1 expression and/or activity, and (ii) increased cGAS/STING signaling pathway activity, and/or (ii) an elevated level of cGAMP in a serum or tumor sample of the subject as compared to a reference level, in step (a) has an increased likelihood of being responsive to treatment with a STING antagonist or a cGAS inhibitor.

9. A method of predicting a subject's responsiveness to a STING antagonist or cGAS inhibitor, the method comprising identifying a subject determined to have a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level, as having an increased likelihood of being responsive to treatment with a STING antagonist or a cGAS inhibitor.

10. The method of any one of claims 1-9, wherein the subject is identified as having a cancer cell having decreased TREX1 level and/or activity.

11. The method of any one of claims 1-9, wherein the subject is identified as having a cancer cell having increased cGAS/STING signaling pathway activity.

12. The method of any one of claims 1-9, wherein the subject is identified as having an elevated level of cGAMP in a serum or tumor sample of the subject as compared to a reference level.

13. The method of any one of claims 1-9, wherein the subject is identified having a cancer cell having both (i) decreased TREX1 level and/or activity and (ii) increased cGAS/STING signaling pathway activity.

14. The method of claim 13, wherein the subject is identified as having an elevated level of cGAMP in a serum or tumor sample of the subject as compared to a reference level.

15. The method of any one of claims 1-13, wherein the TREX1 level is a level of TREX1 protein in the cancer cell.

16. The method of any one of claims 1-13, wherein the identification of the subject as having a cancer cell having a decreased TREX1 level comprises detecting a decreased level of TREX1 protein in the cancer cell.

17. The method of any one of claims 1-13, wherein the TREX1 level is a level of TREX1 mRNA in the cancer cell.

18. The method of any one of claims 1-13, wherein the identification of the subject as having a cancer cell having a decreased TREX1 level comprises detecting a decreased level of TREX1 mRNA in the cancer cell.

19. The method any one of claims 1-13, wherein the decreased TREX1 level and/or activity is a result of TREX1 gene loss in the cancer cell.

20. The method of claim 19, wherein the TREX1 gene loss is loss of one allele of the TREX1 gene.

21. The method of claim 19, wherein the TREX1 gene loss is loss of both alleles of the TREX1 gene.

22. The method of any one of claims 1-13, wherein the identification of the subject as having a cancer cell having decreased TREX1 level and/or activity comprises detecting TREX1 gene loss in the cancer cell.

23. The method of any one of claims 1-13, wherein the decreased TREX1 level and/or activity is a result of one or more amino acid deletions in a protein encoded by a TREX1 gene in the cancer cell.

24. The method of any one of claims 1-13, wherein the identification of the subject as having a cancer cell having decreased TREX1 level and/or activity comprises detecting one or more amino acid deletions in a protein encoded by a TREX1 gene in the cancer cell.

25. The method of any one of claims 1-13, wherein the decreased TREX1 level and/or activity is a result of one or more inactivating amino acid substitutions in a protein encoded by a TREX1 gene in the cancer cell.

26. The method of any one of claims 1-13, wherein the identification of the subject as having a cancer cell having decreased TREX1 expression and/or activity comprises detecting one or more inactivating amino acid substitutions in a protein encoded by a TREX1 gene in the cancer cell.

27. The method of any one of claims 1-13, wherein the increased cGAS/STING signaling pathway activity and/or the elevated level of cGAMP is a result of a decreased level and/or activity of BRCA1 in the cancer cell.

28. The method of claim 27, wherein the decreased level and/or activity of BRCA1 in the cancer cell is a result of a frameshift mutation in a BRCA1 gene.

29. The method of claim 28, wherein the frameshift mutation in a BRCA1 gene is a E111Gfs*3 frameshift insertion.

30. The method of claim 29, wherein the decreased level and/or activity of BRCA1 in the cancer cell is a result of BRCA1 gene loss in the cancer cell.

31. The method of claim 27, wherein the decreased level and/or activity of BRCA1 in the cancer cell is a result of one or more amino acid deletions in a protein encoded by a BRCA1 gene.

32. The method of claim 27, wherein the decreased level and/or activity of BRCA1 in the cancer cell is a result of one or more inactivating amino acid substitutions in a protein encoded by a BRCA1 gene.

33. The method of any one of claims 1-13, wherein the increased cGAS/STING signaling pathway activity is a result of a decreased level and/or activity of BRCA2 gene.

34. The method of claim 33, wherein the decreased level and/or activity of BRCA2 in the cancer cell is a result of a frameshift mutation in a BRCA2 gene.

35. The method of claim 34, wherein the frameshift mutation in a BRCA2 gene is a N1784Kfs*3 frameshift insertion.

36. The method of claim 33, wherein the decreased level and/or activity of BRCA2 in the cancer cell is a result of BRCA2 gene loss in the cancer cell.

37. The method of claim 33, wherein the decreased level and/or activity of BRCA2 in the cancer cell is a result of one or more amino acid deletions in a protein encoded by a BRCA2 gene.

38. The method of claim 33, wherein the decreased level and/or activity of BRCA2 in the cancer cell is a result of one or more inactivating amino acid substitutions in a protein encoded by a BRCA2 gene.

39. The method of any one of claims 1-13, wherein the increased cGAS/STING signaling pathway activity is a result of a decreased level and/or activity of SAMHD1 in the cancer cell.

40. The method of claim 39, wherein the decreased level and/or activity of SAMHD1 in the cancer cell is a result of one or more inactivating amino acid substitutions in a protein encoded by a SAMHD1 gene in the cancer cell.

41. The method of claim 40, wherein the one or more inactivating amino acid substitutions in a protein encoded by a SAMHD1 gene is a V133I amino acid substitution.

42. The method of claim 39, wherein the decreased level and/or activity of SAMHD1 in the cancer cell is a result of SAMHD1 gene loss in the cancer cell.

43. The method of claim 39, wherein the decreased level and/or activity of SAMHD1 in the cancer cell is a result of one or more amino acid deletions in a protein encoded by a SAMHD1 gene.

44. The method of any one of claims 1-13, wherein the increased cGAS/STING signaling pathway activity is a result of a decreased level and/or activity of DNASE2 in the cancer cell.

45. The method of claim 44, wherein the decreased level and/or activity of DNASE2 in the cancer cell is a result of one or more inactivating amino acid substitutions in a protein encoded by a DNASE2 gene in the cancer cell.

46. The method of claim 45, wherein the one or more inactivating amino acid substitutions in a protein encoded by a DNASE2 gene is a R314W amino acid substitution.

47. The method of claim 44, wherein the decreased level and/or activity of DNASE2 in the cancer cell is a result of DNASE2 gene loss in the cancer cell.

48. The method of claim 44, wherein the decreased level and/or activity of DNASE2 in the cancer cell is a result of one or more amino acid deletions in a protein encoded by a DNASE2 gene.

49. The method of any one of claims 1-13, wherein the increased cGAS/STING signaling pathway activity and/or the elevated level of cGAMP is a result of a decreased level and/or activity of BLM in the cancer cell.

50. The method of claim 49, wherein the decreased level and/or activity of BLM in the cancer cell is a result of a frameshift mutation in a BLM gene.

51. The method of claim 50, wherein the frameshift mutation in a BLM gene is a N515Mfs*16 frameshift deletion.

52. The method of claim 49, wherein the decreased level and/or activity of BLM in the cancer cell is a result of BLM gene loss in the cancer cell.

53. The method of claim 49, wherein the decreased level and/or activity of BLM in the cancer cell is a result of one or more amino acid deletions in a protein encoded by a BLM gene.

54. The method of claim 49, wherein the decreased level and/or activity of BLM in the cancer cell is a result of one or more inactivating amino acid substitutions in a protein encoded by a BLM gene.

55. The method of any one of claims 1-13, wherein the increased cGAS/STING signaling pathway activity is a result of a decreased level and/or activity of PARP1 in the cancer cell.

56. The method of claim 55, wherein the decreased level and/or activity of PARP1 in the cancer cell is a result of a frameshift mutation in a PARP1 gene.

57. The method of claim 56, wherein the frameshift mutation in a PARP1 gene is a S507Afs*17 frameshift deletion.

58. The method of claim 55, wherein the decreased level and/or activity of PARP1 in the cancer cell is a result of PARP1 gene loss in the cancer cell.

59. The method of claim 55, wherein the decreased level and/or activity of PARP1 in the cancer cell is a result of one or more amino acid deletions in a protein encoded by a PARP1 gene.

60. The method of claim 55, wherein the decreased level and/or activity of PARP1 in the cancer cell is a result of one or more inactivating amino acid substitutions in a protein encoded by a PARP1 gene.

61. The method of any one of claims 1-13, wherein the increased cGAS/STING signaling pathway activity is a result of a decreased level and/or activity of RPA1 in the cancer cell.

62. The method of claim 61, wherein the decreased level and/or activity of RPA1 in the cancer cell is a result of a mutation that results in aberrant RPA1 mRNA splicing in the cancer cell.

63. The method of claim 62, wherein the mutation that results in aberrant RPA1 mRNA splicing in the cancer cell is a X12 splice mutation.

64. The method of claim 61, wherein the decreased level and/or activity of RPA1 in the cancer cell is a result of RPA1 gene loss in the cancer cell.

65. The method of claim 61, wherein the decreased level and/or activity of RPA1 in the cancer cell is a result of one or more amino acid deletions in a protein encoded by a RPA1 gene.

66. The method of claim 61, wherein the decreased level and/or activity of RPA1 in the cancer cell is a result of one or more inactivating amino acid substitutions in a protein encoded by a RPA1 gene.

67. The method of any one of claims 1-13, wherein the increased cGAS/STING signaling pathway activity is a result of a decreased level and/or activity of RAD51 in the cancer cell.

68. The method of claim 67, wherein the decreased level and/or activity of RAD51 in the cancer cell is a result of one or more inactivating amino acid substitutions in a protein encoded by a RAD51 gene.

69. The method of claim 68, wherein the one or more inactivating amino acid substitutions in a protein encoded by a RAD51 gene is an R254* amino acid substitution.

70. The method of claim 67, wherein the decreased level and/or activity of RAD51 in the cancer cell is a result of RAD51 gene loss in the cancer cell.

71. The method of claim 67, wherein the decreased level and/or activity of RAD51 in the cancer cell is a result of one or more amino acid deletions in a protein encoded by a RAD51 gene.

72. The method of any one of claims 1-13, wherein the increased cGAS/STING signaling pathway activity is a result of an increased level and/or activity of MUS81 in the cancer cell.

73. The method of claim 72, wherein the increased level and/or activity of MUS81 in the cancer cell is a result of MUS81 gene amplification in the cancer cell.

74. The method of claim 72, wherein the increased level and/or activity of MUS81 in the cancer cell is a result of one or more activating amino acid substitutions in a protein encoded by a MUS81 gene.

75. The method of any one of claims 11-13, wherein the increased cGAS/STING signaling pathway activity is a result of an increased level and/or activity of IFI16 in the cancer cell.

76. The method of claim 75, wherein the increased level and/or activity of IFI16 in the cancer cell is a result of IFI16 gene amplification in the cancer cell.

77. The method of claim 75, wherein the increased level and/or activity of IFI16 in the cancer cell is a result of one or more activating amino acid substitutions in a protein encoded by a IFI16 gene.

78. The method of any one of claims 1-13, wherein the increased cGAS/STING signaling pathway activity is a result of an increased level and/or activity of cGAS in the cancer cell.

79. The method of claim 78, wherein the increased level and/or activity of cGAS in the cancer cell is a result of cGAS gene amplification in the cancer cell.

80. The method of claim 78, wherein the increased level and/or activity of cGAS in the cancer cell is a result of one or more activating amino acid substitutions in a protein encoded by a cGAS gene.

81. The method of any one of claims 1-13, wherein the increased cGAS/STING signaling pathway activity is a result of a gain-of-function mutation of STING, with the proviso that the method does not comprise administering a treatment comprising a therapeutically effective amount of a cGAS inhibitor, or a pharmaceutically acceptable salt, solvate, or co-crystal thereof to the identified subject.

82. The method of any one of claims 1-13, wherein the increased cGAS/STING signaling pathway activity is a result of an increased level and/or activity of DDX41 in the cancer cell.

83. The method of claim 82, wherein the increased level and/or activity of DDX41 in the cancer cell is a result of DDX41 gene amplification in the cancer cell.

84. The method of claim 82, wherein the increased level and/or activity of DDX41 in the cancer cell is a result of one or more activating amino acid substitutions in a protein encoded by a DDX41 gene.

85. The method of any one of claims 1-13, wherein the increased cGAS/STING signaling pathway activity is a result of an increased level and/or activity of EXO1 in the cancer cell.

86. The method of claim 85, wherein the increased level and/or activity of EXO1 in the cancer cell is a result of EXO1 gene amplification in the cancer cell.

87. The method of claim 85, wherein the increased level and/or activity of EXO1 in the cancer cell is a result of one or more activating amino acid substitutions in a protein encoded by a EXO1 gene.

88. The method of any one of claims 1-13, wherein the increased cGAS/STING signaling pathway activity is a result of an increased level and/or activity of DNA2 in the cancer cell.

89. The method of claim 88, wherein the increased level and/or activity of DNA2 in the cancer cell is a result of DNA2 gene amplification in the cancer cell.

90. The method of claim 88, wherein the increased level and/or activity of DNA2 in the cancer cell is a result of one or more activating amino acid substitutions in a protein encoded by a DNA2 gene.

91. The method of any one of claims 1-13, wherein the increased cGAS/STING signaling pathway activity is a result of an increased level and/or activity of RBBP8 (CtIP) in the cancer cell.

92. The method of claim 91, wherein the increased level and/or activity of RBBP8 (CtIP) in the cancer cell is a result of RBBP8 (CtIP) gene amplification in the cancer cell.

93. The method of claim 91, wherein the increased level and/or activity of RBBP8 (CtIP) in the cancer cell is a result of one or more activating amino acid substitutions in a protein encoded by a RBBP8 (CtIP) gene.

94. The method of any one of claims 1-13, wherein the increased cGAS/STING signaling pathway activity is a result of an increased level and/or activity of MRE11 in the cancer cell.

95. The method of claim 94, wherein the increased level and/or activity of MRE11 in the cancer cell is a result of MRE11 gene amplification in the cancer cell.

96. The method of claim 94, wherein the increased level and/or activity of MRE11 in the cancer cell is a result of one or more activating amino acid substitutions in a protein encoded by a MRE11 gene.

97. The method of claim 3 or 4, further comprising administering the selected treatment to the subject.

98. The method of claim 8 or 9, further comprising administering a therapeutically effective amount of a STING antagonist or a cGAS inhibitor to a subject identified as having an increased likelihood of being responsive to treatment with a STING antagonist or a cGAS inhibitor.

99. The method of any one of claims 1-98, wherein the subject has been diagnosed or identified as having a cancer.

100. The method of claim 99, wherein the cancer is selected from the group consisting of: renal clear cell carcinoma, uveal melanoma, tongue squamous cell carcinoma, breast cancer, and skin cancer.

101. The method of any one of claims 1-100, wherein the STING antagonist is a compound of any one of Formulas I-X, or a pharmaceutically acceptable salt, solvate, or co-crystal thereof.

102. The method of any one of claims 1-100, wherein the STING antagonist or the cGAS inhibitor is a compound selected from the group consisting of the compounds in Tables 1-10, or a pharmaceutically acceptable salt, solvate, or co-crystal thereof.

What is claimed is:

1. A method of treating a subject in need thereof, the method comprising:
(a) identifying a subject having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level; and
(b) administering a treatment comprising a therapeutically effective amount of a STING antagonist or a cGAS inhibitor, or a pharmaceutically acceptable salt, solvate, or co-crystal thereof to the identified subject.

2. A method of treating a subject in need thereof, the method comprising administering a treatment comprising a therapeutically effective amount of a STING antagonist or a cGAS inhibitor, or a pharmaceutically acceptable salt, solvate, or co-crystal thereof to a subject identified as having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level.

3. A method of selecting a treatment for a subject in need thereof, the method comprising:
(a) identifying a subject having a cancer cell having (i) one or both of (i) decreased TREX1 level and/or activity, and (ii) increased cGAS/STING signaling pathway activity and/or (ii) an elevated level of cGAMP in a serum or tumor sample of the subject as compared to a reference level; and
(b) selecting for the identified subject a treatment comprising a therapeutically effective amount of a STING antagonist or a cGAS inhibitor, or a pharmaceutically acceptable salt, solvate, or co-crystal thereof.

4. A method of selecting a treatment for a subject in need thereof, the method comprising selecting a treatment comprising a therapeutically effective amount of a STING antagonist or cGAS inhibitor, or a pharmaceutically acceptable salt, solvate, or co-crystal thereof for a subject identified as having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level.

5. A method of selecting a subject for treatment, the method comprising:
(a) identifying a subject having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level; and
(b) selecting the identified subject for treatment with a therapeutically effective amount of a STING antagonist or a cGAS inhibitor, or a pharmaceutically acceptable salt, solvate, or co-crystal thereof.

6. A method of selecting a subject for participation in a clinical trial, the method comprising:
(a) identifying a subject having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level; and
(b) selecting the identified subject for participation in a clinical trial that comprises administration of a treatment comprising a therapeutically effective amount of a STING antagonist or a cGAS inhibitor, or a pharmaceutically acceptable salt, solvate, or co-crystal thereof.

7. A method of selecting a subject for participation in a clinical trial, the method comprising selecting a subject identified as having a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level, for participation in a clinical trial that comprises administration of a treatment comprising a therapeutically effective amount of a STING antagonist or a cGAS inhibitor, or a pharmaceutically acceptable salt, solvate, or co-crystal thereof.

8. A method of predicting a subject's responsiveness to a STING antagonist or cGAS inhibitor, the method comprising:
(a) determining that a subject has a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level; and
(b) identifying that the subject determined to have (i) one or both of (i) decreased TREX1 expression and/or activity, and (ii) increased cGAS/STING signaling pathway activity, and/or (ii) an elevated level of cGAMP in a serum or tumor sample of the subject as compared to a reference level, in step (a) has an increased likelihood of being responsive to treatment with a STING antagonist or a cGAS inhibitor.

9. A method of predicting a subject's responsiveness to a STING antagonist or cGAS inhibitor, the method comprising identifying a subject determined to have a cancer cell having (i) one or both of (i) decreased TREX1 level and/or activity, and (ii) increased cGAS/STING signaling pathway activity, and/or (ii) an elevated level of cGAMP in a serum or tumor sample of the subject as compared to a reference level, as having an increased likelihood of being responsive to treatment with a STING antagonist or a cGAS inhibitor.

10. The method of any one of claims 1-9, wherein the subject is identified having a cancer cell having both (i) decreased TREX1 level and/or activity and (ii) increased cGAS/STING signaling pathway activity; and
optionally wherein the subject is identified as having an elevated level of cGAMP in a serum or tumor sample of the subject as compared to a reference level.

11. The method of any one of claims 1-10, wherein the increased cGAS/STING signaling pathway activity and/or the elevated level of cGAMP is a result of a decreased level and/or activity of BRCA1 in the cancer cell.

12. The method of any one of claims 1-11, wherein the increased cGAS/STING signaling pathway activity is a result of a decreased level and/or activity of BRCA2 gene; or a decreased level and/or activity of SAMHD1 in the cancer cell; or a decreased level and/or activity of DNASE2 in the cancer cell; or a decreased level and/or activity of PARP1 in the cancer cell; or a decreased level and/or activity of RPA1 in the cancer cell; or a decreased level and/or activity of RAD51 in the cancer cell; or an increased level and/or activity of MUS81 in the cancer cell; or an increased level and/or activity of IFI16 in the cancer cell; or an increased level and/or activity of cGAS in the cancer cell; or an increased level and/or activity of DDX41 in the cancer cell; or an increased level and/or activity of EXO1 in the cancer cell; an increased level and/or activity of DNA2 in the cancer cell; or an increased level and/or activity of RBBP8 (CtIP) in the cancer cell; or an increased level and/or activity of MRE11 in the cancer cell.

13. The method of any one of claims 1-11, wherein the increased cGAS/STING signaling pathway activity and/or the elevated level of cGAMP is a result of a decreased level and/or activity of BLM in the cancer cell.

14. The method of any one of claims 1-11, wherein the increased cGAS/STING signaling pathway activity is a result of a gain-of-function mutation of STING, with the proviso that the method does not comprise administering a treatment comprising a therapeutically effective amount of a cGAS inhibitor, or a pharmaceutically acceptable salt, solvate, or co-crystal thereof to the identified subject.

15. The method of claim 3 or 4, further comprising administering the selected treatment to the subject.

16. The method of claim 8 or 9, further comprising administering a therapeutically effective amount of a STING antagonist or a cGAS inhibitor to a subject identified as having an increased likelihood of being responsive to treatment with a STING antagonist or a cGAS inhibitor.

17. The method of any one of claims 1-16, wherein the subject has been diagnosed or identified as having a cancer, such as a cancer is selected from the group consisting of: renal clear cell carcinoma, uveal melanoma, tongue squamous cell carcinoma, breast cancer, and skin cancer.

18. The method of any one of claims 1-17, wherein the STING antagonist is a compound of any one of Formulas I-X, or a pharmaceutically acceptable salt, solvate, or co-crystal thereof.

19. The method of any one of claims 1-17, wherein the STING antagonist or the cGAS inhibitor is a compound selected from the group consisting of the compounds in Tables 1-10, or a pharmaceutically acceptable salt, solvate, or co-crystal thereof.


SEQUENCE LISTING (89 sequences)

SEQ ID NO: 1 - DNA, 1140 bp, Homo sapiens
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 2 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 3 - DNA, 852 bp, Homo sapiens
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcggaacctgcagatgacagcagcttctcgctgtcccaggaggttctccggcacctgcggcaggaggaaaaggaagaggttactgtgggcagcttga

SEQ ID NO: 4 - protein, 283 aa, Homo sapiens [sequence omitted]

SEQ ID NO: 5 - DNA, 783 bp, Homo sapiens
atgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 6 - protein, 260 aa, Homo sapiens
MLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 7 - DNA, 735 bp, Homo sapiens
ctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 8 - protein, 244 aa, Homo sapiens
LKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 9 - DNA, 945 bp, Homo sapiens
atgggctcgcaggccctgcccccggggcccatgcagaccctcatctttttcgacatggaggccactggcttgcccttctcccagcccaaggtcacggagctgtgcctgctggctgtccacagatgtgccctggagagcccccccacctctcaggggccacctcccacagttcctccaccaccgcgtgtggtagacaagctctccctgtgtgtggctccggggaaggcctgcagccctgcagccagcgagatcacaggtctgagcacagctgtgctggcagcgcatgggcgtcaatgttttgatgacaacctggccaacctgctcctagccttcctgcggcgccagccacagccctggtgcctggtggcacacaatggtgaccgctacgacttccccctgctccaagcagagctggctatgctgggcctcaccagtgctctggatggtgccttctgtgtggatagcatcactgcgctgaaggccctggagcgagcaagcagcccctcagaacacggcccaaggaagagctatagcctaggcagcatctacactcgcctgtatgggcagtcccctccagactcgcacacggctgagggtgatgtcctggccctgctcagcatctgtcagtggagaccacaggccctgctgcggtgggtggatgctcacgccaggcctttcggcaccatcaggcccatgtatggggtcacagcctctgctaggaccaagccaagaccatctgctgtcacaaccactgcacacctggccacaaccaggaacactagtcccagccttggagagagcaggggtaccaaggatcttcctccagtgaaggaccctggagccctatccagggaggggctgctggccccactgggtctgctggccatcctgaccttggcagtagccacactgtatggactatccctggccacacctggggagtag

SEQ ID NO: 10 - protein, 314 aa, Homo sapiens
MGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTKPRPSAVTTTAHLATTRNTSPSLGESRGTKDLPPVKDPGALSREGLLAPLGLLAILTLAVATLYGLSLATPGE

SEQ ID NO: 11 - DNA, 915 bp, Homo sapiens
atgcagaccctcatctttttcgacatggaggccactggcttgcccttctcccagcccaaggtcacggagctgtgcctgctggctgtccacagatgtgccctggagagcccccccacctctcaggggccacctcccacagttcctccaccaccgcgtgtggtagacaagctctccctgtgtgtggctccggggaaggcctgcagccctgcagccagcgagatcacaggtctgagcacagctgtgctggcagcgcatgggcgtcaatgttttgatgacaacctggccaacctgctcctagccttcctgcggcgccagccacagccctggtgcctggtggcacacaatggtgaccgctacgacttccccctgctccaagcagagctggctatgctgggcctcaccagtgctctggatggtgccttctgtgtggatagcatcactgcgctgaaggccctggagcgagcaagcagcccctcagaacacggcccaaggaagagctatagcctaggcagcatctacactcgcctgtatgggcagtcccctccagactcgcacacggctgagggtgatgtcctggccctgctcagcatctgtcagtggagaccacaggccctgctgcggtgggtggatgctcacgccaggcctttcggcaccatcaggcccatgtatggggtcacagcctctgctaggaccaagccaagaccatctgctgtcacaaccactgcacacctggccacaaccaggaacactagtcccagccttggagagagcaggggtaccaaggatcttcctccagtgaaggaccctggagccctatccagggaggggctgctggccccactgggtctgctggccatcctgaccttggcagtagccacactgtatggactatccctggccacacctggggagtag

SEQ ID NO: 12 - protein, 304 aa, Homo sapiens
MQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTKPRPSAVTTTAHLATTRNTSPSLGESRGTKDLPPVKDPGALSREGLLAPLGLLAILTLAVATLYGLSLATPGE

SEQ ID NO: 13 - protein, 369 aa, Homo sapiens
MGPGARRQGRIVQGRPEMCFCPPPTPLPPLRILTLGTHTPTPCSSPGSAAGTYPTMGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTKPRPSAVTTTAHLATTRNTSPSLGESRGTKDLPPVKDPGALSREGLLAPLGLLAILTLAVATLYGLSLATPGE

SEQ ID NO: 14 - DNA, 5592 bp, Homo sapiens
atggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 15 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 16 - DNA, 5451 bp, Homo sapiens
atgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 17 - protein, 1816 aa, Homo sapiens
MLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 18 - DNA, 2280 bp, Homo sapiens
atggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 19 - protein, 759 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGEAASGCESETSVSEDCSGLSSQSDILTTQQRDTMQHNLIKLQQEMAELEAVLEQHGSQPSNSYPSIISDSSALEDLRNPEQSTSEKVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 20 - DNA, 2100 bp, Homo sapiens
atggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagggtgtccacccaattgtggttgtgcagccagatgcctggacagaggacaatggcttccatgcaattgggcagatgtgtga

SEQ ID NO: 21 - protein, 699 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGEAASGCESETSVSEDCSGLSSQSDILTTQQRDTMQHNLIKLQQEMAELEAVLEQHGSQPSNSYPSIISDSSALEDLRNPEQSTSEKVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTGCPPNCGCAARCLDRGQWLPCNWADV

SEQ ID NO: 22 - DNA, 5655 bp, Homo sapiens
atggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagattcgcatatacatggccaaaggaacaactccatgttttctaaaaggcctagagaacatatatcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 23 - protein, 1884 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKDSHIHGQRNNSMFSKRPREHISVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 24 - DNA, 10257 bp, Homo sapiens
atgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcaaatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacatcttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctattgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtgtcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagtagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaa

SEQ ID NO: 25 - protein, 3418 aa, Homo sapiens [sequence omitted]

SEQ ID NO: 26 - DNA, 1881 bp, Homo sapiens
atgcagcgagccgattccgagcagccctccaagcgtccccgttgcgatgacagcccgagaaccccctcaaacaccccttccgcagaggcagactggtccccgggcctggaactccatcccgactacaagacatggggtccggagcaggtgtgctccttcctcaggcgcggtggctttgaagagccggtgctgctgaagaacatccgagaaaatgaaatcacaggcgcattactgccttgtcttgatgagtctcgttttgaaaatcttggagtaagttccttgggggagaggaagaagctgcttagttatatccagcgattggttcaaatccacgttgatacaatgaaggtaattaatgatcctatccatggccacattgagctccaccctctcctcgtccgaatcattgatacacctcaatttcaacgtcttcgatacatcaaacagctgggaggtggttactatgtttttccaggagcttcacacaatcgatttgagcatagtctaggggtggggtatctagcaggatgtctagttcacgcactgggtgaaaaacaaccagagctgcagataagtgaacgagatgttctctgtgttcagattgctggactttgtcatgatctcggtcatgggccattttctcacatgtttgatggacgatttattccacttgctcgcccggaggtgaaatggacgcatgaacaaggctcagttatgatgtttgagcaccttattaattctaatggaattaagcctgtcatggaacaatatggtctcatccctgaagaagatatttgctttataaaggaacaaattgtaggaccacttgaatcacctgtcgaagattcattgtggccatataaagggcgtcctgaaaacaaaagcttcctttatgagatagtatctaataaaagaaatggcattgatgtggacaaatgggattattttgccagggactgccatcatcttggaatccaaaataattttgattacaagcgctttattaagtttgcccgtgtctgtgaagtagacaatgagttgcgtatttgtgctagagataaggaagttggaaatctgtatgacatgttccacactcgcaactctttacaccgtagagcttatcaacacaaagttggcaacattattgatacaatgattacagatgctttcctcaaagcagatgactacatagagattacaggtgctggaggaaaaaagtatcgcatttctacagcaattgacgacatggaagcctatactaagctgacagataacatttttctggagattttatactctactgatcccaaattgaaagacgcacgagagattttaaaacaaattgaataccgtaatctattcaagtatgtgggtgagacgcagccaacaggacaaataaagattaaaagggaggactatgaatctcttccaaaagaggttgccagtgctaaacccaaagtattgctagacgtgaaactgaaggctgaagattttatagtggatgttatcaacatggattatggaatgcaagaaaagaatccaattgatcatgttagcttctattgtaagactgcccccaacagagcaatcaggattactaaaaaccaggtttcacaacttctgccagagaaatttgcagagcagctgattcgagtatattgtaagaaggtggacagaaagagtttgtatgccgcaagacaatattttgttcagtggtgtgcagacagaaatttcaccaagccgcaggatggcgatgttatagccccactcataacacctcaaaaaaaggaatggaacgacagtacttcagtccaaaatccaactcgcctccgagaagcatccaaaagcagagtccagctttttaaagatgacccaatgtga

SEQ ID NO: 27 - protein, 626 aa, Homo sapiens
MQRADSEQPSKRPRCDDSPRTPSNTPSAEADWSPGLELHPDYKTWGPEQVCSFLRRGGFEEPVLLKNIRENEITGALLPCLDESRFENLGVSSLGERKKLLSYIQRLVQIHVDTMKVINDPIHGHIELHPLLVRIIDTPQFQRLRYIKQLGGGYYVFPGASHNRFEHSLGVGYLAGCLVHALGEKQPELQISERDVLCVQIAGLCHDLGHGPFSHMFDGRFIPLARPEVKWTHEQGSVMMFEHLINSNGIKPVMEQYGLIPEEDICFIKEQIVGPLESPVEDSLWPYKGRPENKSFLYEIVSNKRNGIDVDKWDYFARDCHHLGIQNNFDYKRFIKFARVCEVDNELRICARDKEVGNLYDMFHTRNSLHRRAYQHKVGNIIDTMITDAFLKADDYIEITGAGGKKYRISTAIDDMEAYTKLTDNIFLEILYSTDPKLKDAREILKQIEYRNLFKYVGETQPTGQIKIKREDYESLPKEVASAKPKVLLDVKLKAEDFIVDVINMDYGMQEKNPIDHVSFYCKTAPNRAIRITKNQVSQLLPEKFAEQLIRVYCKKVDRKSLYAARQYFVQWCADRNFTKPQDGDVIAPLITPQKKEWNDSTSVQNPTRLREASKSRVQLFKDDPM

SEQ ID NO: 28 - DNA, 1776 bp, Homo sapiens
atgcagcgagccgattccgagcagccctccaagcgtccccgttgcgatgacagcccgagaaccccctcaaacaccccttccgcagaggcagactggtccccgggcctggaactccatcccgactacaagacatggggtccggagcaggtgtgctccttcctcaggcgcggtggctttgaagagccggtgctgctgaagaacatccgagaaaatgaaatcacaggcgcattactgccttgtcttgatgagtctcgttttgaaaatcttggagtaagttccttgggggagaggaagaagctgcttagttatatccagcgattggttcaaatccacgttgatacaatgaaggtaattaatgatcctatccatggccacattgagctccaccctctcctcgtccgaatcattgatacacctcaatttcaacgtcttcgatacatcaaacagctgggaggtggttactatgtttttccaggagcttcacacaatcgatttgagcatagtctaggggtggggtatctagcaggatgtctagttcacgcactgggtgaaaaacaaccagagctgcagataagtgaacgagatgttctctgtgttcagattgctggactttgtcatgatctcggtcatgggccattttctcacatgtttgatggacgatttattccacttgctcgcccggaggtgaaatggacgcatgaacaaggctcagttatgatgtttgagcaccttattaattctaatggaattaagcctgtcatggaacaatatggtctcatccctgaagaagatatttgctttataaaggaacaaattgtaggaccacttgaatcacctgtcgaagattcattgtggccatataaagggcgtcctgaaaacaaaagcttcctttatgagatagtatctaataaaagaaatggcattgatgtggacaaatgggattattttgccagggactgccatcatcttggaatccaaaataattttgattacaagcgctttattaagtttgcccgtgtctgtgaagtagacaatgagttgcgtatttgtgctagagataaggaagttggaaatctgtatgacatgttccacactcgcaactctttacaccgtagagcttatcaacacaaagttggcaacattattgatacaatgattacagatgctttcctcaaagcagatgactacatagagattacaggtgctggaggaaaaaagtatcgcatttctacagcaattgacgacatggaagcctatactaagctgacagataacatttttctggagattttatactctactgatcccaaattgaaagacgcacgagagattttaaaacaaattgaataccgtaatctattcaagtatgtgggtgagacgcagccaacaggacaaataaagattaaaagggaggactatgaatctcttccaaaagaggttgccagtgctaaacccaaagtattgctagacgtgaaactgaaggctgaagattttatagtggatgtttcacaacttctgccagagaaatttgcagagcagctgattcgagtatattgtaagaaggtggacagaaagagtttgtatgccgcaagacaatattttgttcagtggtgtgcagacagaaatttcaccaagccgcaggatggcgatgttatagccccactcataacacctcaaaaaaaggaatggaacgacagtacttcagtccaaaatccaactcgcctccgagaagcatccaaaagcagagtccagctttttaaagatgacccaatgtga

SEQ ID NO: 29 - protein, 591 aa, Homo sapiens
MQRADSEQPSKRPRCDDSPRTPSNTPSAEADWSPGLELHPDYKTWGPEQVCSFLRRGGFEEPVLLKNIRENEITGALLPCLDESRFENLGVSSLGERKKLLSYIQRLVQIHVDTMKVINDPIHGHIELHPLLVRIIDTPQFQRLRYIKQLGGGYYVFPGASHNRFEHSLGVGYLAGCLVHALGEKQPELQISERDVLCVQIAGLCHDLGHGPFSHMFDGRFIPLARPEVKWTHEQGSVMMFEHLINSNGIKPVMEQYGLIPEEDICFIKEQIVGPLESPVEDSLWPYKGRPENKSFLYEIVSNKRNGIDVDKWDYFARDCHHLGIQNNFDYKRFIKFARVCEVDNELRICARDKEVGNLYDMFHTRNSLHRRAYQHKVGNIIDTMITDAFLKADDYIEITGAGGKKYRISTAIDDMEAYTKLTDNIFLEILYSTDPKLKDAREILKQIEYRNLFKYVGETQPTGQIKIKREDYESLPKEVASAKPKVLLDVKLKAEDFIVDVSQLLPEKFAEQLIRVYCKKVDRKSLYAARQYFVQWCADRNFTKPQDGDVIAPLITPQKKEWNDSTSVQNPTRLREASKSRVQLFKDDPM

SEQ ID NO: 30 - DNA, 1770 bp, Homo sapiens
atgcagcgagccgattccgagcagccctccaagcgtccccgttgcgatgacagcccgagaaccccctcaaacaccccttccgcagaggcagactggtccccgggcctggaactccatcccgactacaagacatggggtccggagcaggtgtgctccttcctcaggcgcggtggctttgaagagccggtgctgctgaagaacatccgagaaaatgaaatcacaggcgcattactgccttgtcttgatgagtctcgttttgaaaatcttggagtaagttccttgggggagaggaagaagctgcttagttatatccagcgattggttcaaatccacgttgatacaatgaaggtaattaatgatcctatccatggccacattgagctccaccctctcctcgtccgaatcattgatacacctcaatttcaacgtcttcgatacatcaaacagctgggaggtggttactatgtttttccaggagcttcacacaatcgatttgagcatagtctaggggtggggtatctagcaggatgtctagttcacgcactgggtgaaaaacaaccagagctgcagataagtgaacgagatgttctctgtgttcagattgctggactttgtcatgatctcggtcatgggccattttctcacatgtttgatggacgatttattccacttgctcgcccggaggtgaaatggacgcatgaacaaggctcagttatgatgtttgagcaccttattaattctaatggaattaagcctgtcatggaacaatatggtctcatccctgaagaagatatttgctttataaaggaacaaattgtaggaccacttgaatcacctgtcgaagattcattgtggccatataaagggcgtcctgaaaacaaaagcttcctttatgagatagtatctaataaaagaaatggcattgatgtggacaaatgggattattttgccagggactgccatcatcttggaatccaaaataattttgattacaagcgctttattaagtttgcccgtgtctgtgaagtagacaatgagttgcgtatttgtgctagagataaggaagttggaaatctgtatgacatgttccacactcgcaactctttacaccgtagagcttatcaacacaaagttggcaacattattgatacaatgattacagatgctttcctcaaagcagatgactacatagagattacaggtgctggaggaaaaaagtatcgcatttctacagcaattgacgacatggaagcctatactaagctgacagataacatttttctggagattttatactctactgatcccaaattgaaagacgcacgagagattttaaaacaaattgaataccgtaatctattcaagtatgtgggtgagacgcagccaacaggacaaataaagattaaaagggaggactatgaatctcttccaaaagaggttgccagtgctaaacccaaagtattgctagacgtgaaactgaaggctgaagattttatagtggatgttatcaacatggattatggaatgcaagaaaagaatccaattgatcatgttagcttctattgtaagactgcccccaacagagcaatcaggattactaaaaaccaggtttcacaacttctgccagagaaatttgcagagcagctgattcgagtatattgtaagaaggtggacagaaagagtttgtatgccgcaagacaatattttgttcagtggtgtgcagacagaaatttcaccaagccgcagtctcccaccagagcctcccactga

SEQ ID NO: 31 - protein, 589 aa, Homo sapiens
MQRADSEQPSKRPRCDDSPRTPSNTPSAEADWSPGLELHPDYKTWGPEQVCSFLRRGGFEEPVLLKNIRENEITGALLPCLDESRFENLGVSSLGERKKLLSYIQRLVQIHVDTMKVINDPIHGHIELHPLLVRIIDTPQFQRLRYIKQLGGGYYVFPGASHNRFEHSLGVGYLAGCLVHALGEKQPELQISERDVLCVQIAGLCHDLGHGPFSHMFDGRFIPLARPEVKWTHEQGSVMMFEHLINSNGIKPVMEQYGLIPEEDICFIKEQIVGPLESPVEDSLWPYKGRPENKSFLYEIVSNKRNGIDVDKWDYFARDCHHLGIQNNFDYKRFIKFARVCEVDNELRICARDKEVGNLYDMFHTRNSLHRRAYQHKVGNIIDTMITDAFLKADDYIEITGAGGKKYRISTAIDDMEAYTKLTDNIFLEILYSTDPKLKDAREILKQIEYRNLFKYVGETQPTGQIKIKREDYESLPKEVASAKPKVLLDVKLKAEDFIVDVINMDYGMQEKNPIDHVSFYCKTAPNRAIRITKNQVSQLLPEKFAEQLIRVYCKKVDRKSLYAARQYFVQWCADRNFTKPQSPTRASH

SEQ ID NO: 32 - DNA, 1083 bp, Homo sapiens
atgatcccgctgctgctggcagcgctgctgtgcgtccccgccggggccctgacctgctacggggactccgggcagcctgtagactggttcgtggtctacaagctgccagctcttagagggtccggggaggcggcgcagagagggctgcagtacaagtatctggacgagagctccggaggctggcgggacggcagggcactcatcaacagcccggagggggccgtgggccgaagcctgcagccgctgtaccggagcaacaccagccagctcgccttcctgctctacaatgaccaaccgcctcaacccagcaaggctcaggactcttccatgcgtgggcacacgaagggtgtcctgctccttgaccacgatgggggcttctggctggtccacagtgtacctaacttccctccaccggcctcctctgctgcatacagctggcctcatagcgcctgtacctacgggcagaccctgctctgtgtgtcttttcccttcgctcagttctcgaagatgggcaagcagctgacctacacctacccctgggtctataactaccagctggaagggatctttgcccaggaattccccgacttggagaatgtggtcaagggccaccacgttagccaagaaccctggaacagcagcatcacactcacatcccaggccggggctgttttccagagctttgccaagttcagcaaatttggagatgacctgtactccggctggttggcagcagcccttggtaccaacctgcaggtccagttctggcacaaaactgtaggcatcctgccctctaactgctcggatatctggcaggttctgaatgtgaaccagatagctttccctggaccagccggcccaagcttcaacagcacagaggaccactccaaatggtgcgtgtccccaaaagggccctggacctgcgtgggtgacatgaatcggaaccagggagaggagcaacggggtgggggcacactgtgtgcccagctgccagccctctggaaagccttccagccgctggtgaagaactaccagccctgtaatggcatggccaggaagcccagcagagcttataagatctaa

SEQ ID NO: 33 - protein, 360 aa, Homo sapiens
MIPLLLAALLCVPAGALTCYGDSGQPVDWFVVYKLPALRGSGEAAQRGLQYKYLDESSGGWRDGRALINSPEGAVGRSLQPLYRSNTSQLAFLLYNDQPPQPSKAQDSSMRGHTKGVLLLDHDGGFWLVHSVPNFPPPASSAAYSWPHSACTYGQTLLCVSFPFAQFSKMGKQLTYTYPWVYNYQLEGIFAQEFPDLENVVKGHHVSQEPWNSSITLTSQAGAVFQSFAKFSKFGDDLYSGWLAAALGTNLQVQFWHKTVGILPSNCSDIWQVLNVNQIAFPGPAGPSFNSTEDHSKWCVSPKGPWTCVGDMNRNQGEEQRGGGTLCAQLPALWKAFQPLVKNYQPCNGMARKPSRAYKI

SEQ ID NO: 34 - DNA, 1029 bp, Homo sapiens
tgctacggggactccgggcagcctgtagactggttcgtggtctacaagctgccagctcttagagggtccggggaggcggcgcagagagggctgcagtacaagtatctggacgagagctccggaggctggcgggacggcagggcactcatcaacagcccggagggggccgtgggccgaagcctgcagccgctgtaccggagcaacaccagccagctcgccttcctgctctacaatgaccaaccgcctcaacccagcaaggctcaggactcttccatgcgtgggcacacgaagggtgtcctgctccttgaccacgatgggggcttctggctggtccacagtgtacctaacttccctccaccggcctcctctgctgcatacagctggcctcatagcgcctgtacctacgggcagaccctgctctgtgtgtcttttcccttcgctcagttctcgaagatgggcaagcagctgacctacacctacccctgggtctataactaccagctggaagggatctttgcccaggaattccccgacttggagaatgtggtcaagggccaccacgttagccaagaaccctggaacagcagcatcacactcacatcccaggccggggctgttttccagagctttgccaagttcagcaaatttggagatgacctgtactccggctggttggcagcagcccttggtaccaacctgcaggtccagttctggcacaaaactgtaggcatcctgccctctaactgctcggatatctggcaggttctgaatgtgaaccagatagctttccctggaccagccggcccaagcttcaacagcacagaggaccactccaaatggtgcgtgtccccaaaagggccctggacctgcgtgggtgacatgaatcggaaccagggagaggagcaacggggtgggggcacactgtgtgcccagctgccagccctctggaaagccttccagccgctggtgaagaactaccagccctgtaatggcatggccaggaagcccagcagagcttataagatctaa

SEQ ID NO: 35 - protein, 342 aa, Homo sapiens
CYGDSGQPVDWFVVYKLPALRGSGEAAQRGLQYKYLDESSGGWRDGRALINSPEGAVGRSLQPLYRSNTSQLAFLLYNDQPPQPSKAQDSSMRGHTKGVLLLDHDGGFWLVHSVPNFPPPASSAAYSWPHSACTYGQTLLCVSFPFAQFSKMGKQLTYTYPWVYNYQLEGIFAQEFPDLENVVKGHHVSQEPWNSSITLTSQAGAVFQSFAKFSKFGDDLYSGWLAAALGTNLQVQFWHKTVGILPSNCSDIWQVLNVNQIAFPGPAGPSFNSTEDHSKWCVSPKGPWTCVGDMNRNQGEEQRGGGTLCAQLPALWKAFQPLVKNYQPCNGMARKPSRAYKI

SEQ ID NO: 36 - DNA, 4254 bp, Homo sapiens
atggctgctgttcctcaaaataatctacaggagcaactagaacgtcactcagccagaacacttaataataaattaagtctttcaaaaccaaaattttcaggtttcacttttaaaaagaaaacatcttcagataacaatgtatctgtaactaatgtgtcagtagcaaaaacacctgtattaagaaataaagatgttaatgttaccgaagacttttccttcagtgaacctctacccaacaccacaaatcagcaaagggtcaaggacttctttaaaaatgctccagcaggacaggaaacacagagaggtggatcaaaatcattattgccagatttcttgcagactccgaaggaagttgtatgcactacccaaaacacaccaactgtaaagaaatcccgggatactgctctcaagaaattagaatttagttcttcaccagattctttaagtaccatcaatgattgggatgatatggatgactttgatacttctgagacttcaaaatcatttgttacaccaccccaaagtcactttgtaagagtaagcactgctcagaaatcaaaaaagggtaagagaaacttttttaaagcacagctttatacaacaaacacagtaaagactgatttgcctccaccctcctctgaaagcgagcaaatagatttgactgaggaacagaaggatgactcagaatggttaagcagcgatgtgatttgcatcgatgatggccccattgctgaagtgcatataaatgaagatgctcaggaaagtgactctctgaaaactcatttggaagatgaaagagataatagcgaaaagaagaagaatttggaagaagctgaattacattcaactgagaaagttccatgtattgaatttgatgatgatgattatgatacggattttgttccaccttctccagaagaaattatttctgcttcttcttcctcttcaaaatgccttagtacgttaaaggaccttgacacctctgacagaaaagaggatgttcttagcacatcaaaagatcttttgtcaaaacctgagaaaatgagtatgcaggagctgaatccagaaaccagcacagactgtgacgctagacagataagtttacagcagcagcttattcatgtgatggagcacatctgtaaattaattgatactattcctgatgataaactgaaacttttggattgtgggaacgaactgcttcagcagcggaacataagaaggaaacttctaacggaagtagattttaataaaagtgatgccagtcttcttggctcattgtggagatacaggcctgattcacttgatggccctatggagggtgattcctgccctacagggaattctatgaaggagttaaatttttcacaccttccctcaaattctgtttctcctggggactgtttactgactaccaccctaggaaagacaggattctctgccaccaggaagaatctttttgaaaggcctttattcaatacccatttacagaagtcctttgtaagtagcaactgggctgaaacaccaagactaggaaaaaaaaatgaaagctcttatttcccaggaaatgttctcacaagcactgctgtgaaagatcagaataaacatactgcttcaataaatgacttagaaagagaaacccaaccttcctatgatattgataattttgacatagatgactttgatgatgatgatgactgggaagacataatgcataatttagcagccagcaaatcttccacagctgcctatcaacccatcaaggaaggtcggccaattaaatcagtatcagaaagactttcctcagccaagacagactgtcttccagtgtcatctactgctcaaaatataaacttctcagagtcaattcagaattatactgacaagtcagcacaaaatttagcatccagaaatctgaaacatgagcgtttccaaagtcttagttttcctcatacaaaggaaatgatgaagatttttcataaaaaatttggcctgcataattttagaactaatcagctagaggcgatcaatgctgcactgcttggtgaagactgttttatcctgatgccgactggaggtggtaagagtttgtgttaccagctccctgcctgtgtttctcctggggtcactgttgtcatttctcccttgagatcacttatcgtagatcaagtccaaaagctgacttccttggatattccagctacatatctgacaggtgataagactgactcagaagctacaaatatttacctccagttatcaaaaaaagacccaatcataaaacttctatatgtcactccagaaaagatctgtgcaagtaacagactcatttctactctggagaatctctatgagaggaagctcttggcacgttttgttattgatgaagcacattgtgtcagtcagtggggacatgattttcgtcaagattacaaaagaatgaatatgcttcgccagaagtttccttctgttccggtgatggctcttacggccacagctaatcccagggtacagaaggacatcctgactcagctgaagattctcagacctcaggtgtttagcatgagctttaacagacataatctgaaatactatgtattaccgaaaaagcctaaaaaggtggcatttgattgcctagaatggatcagaaagcaccacccatatgattcagggataatttactgcctctccaggcgagaatgtgacaccatggctgacacgttacagagagatgggctcgctgctcttgcttaccatgctggcctcagtgattctgccagagatgaagtgcagcagaagtggattaatcaggatggctgtcaggttatctgtgctacaattgcatttggaatggggattgacaaaccggacgtgcgatttgtgattcatgcatctctccctaaatctgtggagggttactaccaagaatctggcagagctggaagagatggggaaatatctcactgcctgcttttctatacctatcatgatgtgaccagactgaaaagacttataatgatggaaaaagatggaaaccatcatacaagagaaactcacttcaataatttgtatagcatggtacattactgtgaaaatataacggaatgcaggagaatacagcttttggcctactttggtgaaaatggatttaatcctgatttttgtaagaaacacccagatgtttcttgtgataattgctgtaaaacaaaggattataaaacaagagatgtgactgacgatgtgaaaagtattgtaagatttgttcaagaacatagttcatcacaaggaatgagaaatataaaacatgtaggtccttctggaagatttactatgaatatgctggtcgacattttcttggggagtaagagtgcaaaaatccagtcaggtatatttggaaaaggatctgcttattcacgacacaatgccgaaagactttttaaaaagctgatacttgacaagattttggatgaagacttatatatcaatgccaatgaccaggcgatcgcttatgtgatgctcggaaataaagcccaaactgtactaaatggcaatttaaaggtagactttatggaaacagaaaattccagcagtgtgaaaaaacaaaaagcgttagtagcaaaagtgtctcagagggaagagatggttaaaaaatgtcttggagaacttacagaagtctgcaaatctctggggaaagtttttggtgtccattacttcaatatttttaataccgtcactctcaagaagcttgcagaatctttatcttctgatcctgaggttttgcttcaaattgatggtgttactgaagacaaactggaaaaatatggtgcggaagtgatttcagtattacagaaatactctgaatggacatcgccagctgaagacagttccccagggataagcctgtccagcagcagaggccccggaagaagtgccgctgaggagctcgacgaggaaatacccgtatcttcccactactttgcaagtaaaaccagaaatgaaaggaagaggaaaaagatgccagcctcccaaaggtctaagaggagaaaaactgcttccagtggttccaaggcaaagggggggtctgccacatgtagaaagatatcttccaaaacgaaatcctccagcatcattggatccagttcagcctcacatacttctcaagcgacatcaggagccaatagcaaattggggattatggctccaccgaagcctataaatagaccgtttcttaagccttcatatgcattctcataa

SEQ ID NO: 37 - protein, 1417 aa, Homo sapiens
MAAVPQNNLQEQLERHSARTLNNKLSLSKPKFSGFTFKKKTSSDNNVSVTNVSVAKTPVLRNKDVNVTEDFSFSEPLPNTTNQQRVKDFFKNAPAGQETQRGGSKSLLPDFLQTPKEVVCTTQNTPTVKKSRDTALKKLEFSSSPDSLSTINDWDDMDDFDTSETSKSFVTPPQSHFVRVSTAQKSKKGKRNFFKAQLYTTNTVKTDLPPPSSESEQIDLTEEQKDDSEWLSSDVICIDDGPIAEVHINEDAQESDSLKTHLEDERDNSEKKKNLEEAELHSTEKVPCIEFDDDDYDTDFVPPSPEEIISASSSSSKCLSTLKDLDTSDRKEDVLSTSKDLLSKPEKMSMQELNPETSTDCDARQISLQQQLIHVMEHICKLIDTIPDDKLKLLDCGNELLQQRNIRRKLLTEVDFNKSDASLLGSLWRYRPDSLDGPMEGDSCPTGNSMKELNFSHLPSNSVSPGDCLLTTTLGKTGFSATRKNLFERPLFNTHLQKSFVSSNWAETPRLGKKNESSYFPGNVLTSTAVKDQNKHTASINDLERETQPSYDIDNFDIDDFDDDDDWEDIMHNLAASKSSTAAYQPIKEGRPIKSVSERLSSAKTDCLPVSSTAQNINFSESIQNYTDKSAQNLASRNLKHERFQSLSFPHTKEMMKIFHKKFGLHNFRTNQLEAINAALLGEDCFILMPTGGGKSLCYQLPACVSPGVTVVISPLRSLIVDQVQKLTSLDIPATYLTGDKTDSEATNIYLQLSKKDPIIKLLYVTPEKICASNRLISTLENLYERKLLARFVIDEAHCVSQWGHDFRQDYKRMNMLRQKFPSVPVMALTATANPRVQKDILTQLKILRPQVFSMSFNRHNLKYYVLPKKPKKVAFDCLEWIRKHHPYDSGIIYCLSRRECDTMADTLQRDGLAALAYHAGLSDSARDEVQQKWINQDGCQVICATIAFGMGIDKPDVRFVIHASLPKSVEGYYQESGRAGRDGEISHCLLFYTYHDVTRLKRLIMMEKDGNHHTRETHFNNLYSMVHYCENITECRRIQLLAYFGENGFNPDFCKKHPDVSCDNCCKTKDYKTRDVTDDVKSIVRFVQEHSSSQGMRNIKHVGPSGRFTMNMLVDIFLGSKSAKIQSGIFGKGSAYSRHNAERLFKKLILDKILDEDLYINANDQAIAYVMLGNKAQTVLNGNLKVDFMETENSSSVKKQKALVAKVSQREEMVKKCLGELTEVCKSLGKVFGVHYFNIFNTVTLKKLAESLSSDPEVLLQIDGVTEDKLEKYGAEVISVLQKYSEWTSPAEDSSPGISLSSSRGPGRSAAEELDEEIPVSSHYFASKTRNERKRKKMPASQRSKRRKTASSGSKAKGGSATCRKISSKTKSSSIIGSSSASHTSQATSGANSKLGIMAPPKPINRPFLKPSYAFS

SEQ ID NO: 38 - DNA, 3861 bp, Homo sapiens
atggctgctgttcctcaaaataatctacaggagcaactagaacgtcactcagccagaacacttaataataaattaagtctttcaaaaccaaaattttcaggtttcacttttaaaaagaaaacatcttcagataacaatgtatctgtaactaatgtgtcagtagcaaaaacacctgtattaagaaataaagatgttaatgttaccgaagacttttccttcagtgaacctctacccaacaccacaaatcagcaaagggtcaaggacttctttaaaaatgctccagcaggacaggaaacacagagaggtggatcaaaatcattattgccagatttcttgcagactccgaaggaagttgtatgcactacccaaaacacaccaactgtaaagaaatcccgggatactgctctcaagaaattagaatttagttcttcaccagattctttaagtaccatcaatgattgggatgatatggatgactttgatacttctgagacttcaaaatcatttgttacaccaccccaaagtcactttgtaagagtaagcactgctcagaaatcaaaaaagggtaagagaaacttttttaaagcacagctttatacaacaaacacagtaaagactgatttgcctccaccctcctctgaaagcgagcaaatagatttgactgaggaacagaaggatgactcagaatggttaagcagcgatgtgatttgcatcgatgatggccccattgctgaagtgcatataaatgaagatgctcaggaaagtgactctctgaaaactcatttggaagatgaaagagataatagcgaaaagaagaagaatttggaagaagctgaattacattcaactgagaaagttccatgtattgaatttgatgatgatgattatgatacggattttgttccaccttctccagaagaaattatttctgcttcttcttcctcttcaaaatgccttagtacgttaaaggaccttgacacctctgacagaaaagaggatgttcttagcacatcaaaagatcttttgtcaaaacctgagaaaatgagtatgcaggagctgaatccagaaaccagcacagactgtgacgctagacagataagtttacagcagcagcttattcatgtgatggagcacatctgtaaattaattgatactattcctgatgataaactgaaacttttggattgtgggaacgaactgcttcagcagcggaacataagaaggaaacttctaacggaagtagattttaataaaagtgatgccagtcttcttggctcattgtggagatacaggcctgattcacttgatggccctatggagggtgattcctgccctacagggaattctatgaaggagttaaatttttcacaccttccctcaaattctgtttctcctggggactgtttactgactaccaccctaggaaagacaggattctctgccaccaggaagaatctttttgaaaggcctttattcaatacccatttacagaagtcctttgtaagtagcaactgggctgaaacaccaagactaggaaaaaaaaatgaaagctcttatttcccaggaaatgttctcacaagcactgctgtgaaagatcagaataaacatactgcttcaataaatgacttagaaagagaaacccaaccttcctatgatattgataattttgacatagatgactttgatgatgatgatgactgggaagacataatgcataatttagcagccagcaaatcttccacagctgcctatcaacccatcaaggaaggtcggccaattaaatcagtatcagaaagactttcctcagccaagacagactgtcttccagtgtcatctactgctcaaaatataaacttctcagagtcaattcagaattatactgacaagtcagcacaaaatttagcatccagaaatctgaaacatgagcgtttccaaagtcttagttttcctcatacaaaggaaatgatgaagatttttcataaaaaatttggcctgcataattttagaactaatcagctagaggcgatcaatgctgcactgcttggtgaagactgttttatcctgatgccgactggaggtggtaagagtttgtgttaccagctccctgcctgtgtttctcctggggtcactgttgtcatttctcccttgagatcacttatcgtagatcaagtccaaaagctgacttccttggatattccagctacatatctgacaggtgataagactgactcagaagctacaaatatttacctccagttatcaaaaaaagacccaatcataaaacttctatatgtcactccagaaaagatctgtgcaagtaacagactcatttctactctggagaatctctatgagaggaagctcttggcacgttttgttattgatgaagcacattgtgtcagtcagtggggacatgattttcgtcaagattacaaaagaatgaatatgcttcgccagaagtttccttctgttccggtgatggctcttacggccacagctaatcccagggtacagaaggacatcctgactcagctgaagattctcagacctcaggtgtttagcatgagctttaacagacataatctgaaatactatgtattaccgaaaaagcctaaaaaggtggcatttgattgcctagaatggatcagaaagcaccacccatatgattcagggataatttactgcctctccaggcgagaatgtgacaccatggctgacacgttacagagagatgggctcgctgctcttgcttaccatgctggcctcagtgattctgccagagatgaagtgcagcagaagtggattaatcaggatggctgtcaggttatctgtgctacaattgcatttggaatggggattgacaaaccggacgtgcgatttgtgattcatgcatctctccctaaatctgtggagggttactaccaagaatctggcagagctggaagagatggggaaatatctcactgcctgcttttctatacctatcatgatgtgaccagactgaaaagacttataatgatggaaaaagatggaaaccatcatacaagagaaactcacttcaataatttgtatagcatggtacattactgtgaaaatataacggaatgcaggagaatacagcttttggcctactttggtgaaaatggatttaatcctgatttttgtaagaaacacccagatgtttcttgtgataattgctgtaaaacaaaggattataaaacaagagatgtgactgacgatgtgaaaagtattgtaagatttgttcaagaacatagttcatcacaaggaatgagaaatataaaacatgtaggtccttctggaagatttactatgaatatgctggtcgacattttcttggaatctttatcttctgatcctgaggttttgcttcaaattgatggtgttactgaagacaaactggaaaaatatggtgcggaagtgatttcagtattacagaaatactctgaatggacatcgccagctgaagacagttccccagggataagcctgtccagcagcagaggccccggaagaagtgccgctgaggagctcgacgaggaaatacccgtatcttcccactactttgcaagtaaaaccagaaatgaaaggaagaggaaaaagatgccagcctcccaaaggtctaagaggagaaaaactgcttccagtggttccaaggcaaagggggggtctgccacatgtagaaagatatcttccaaaacgaaatcctccagcatcattggatccagttcagcctcacatacttctcaagcgacatcaggagccaatagcaaattggggattatggctccaccgaagcctataaatagaccgtttcttaagccttcatatgcattctcataa

SEQ ID NO: 39 - protein, 1286 aa, Homo sapiens
MAAVPQNNLQEQLERHSARTLNNKLSLSKPKFSGFTFKKKTSSDNNVSVTNVSVAKTPVLRNKDVNVTEDFSFSEPLPNTTNQQRVKDFFKNAPAGQETQRGGSKSLLPDFLQTPKEVVCTTQNTPTVKKSRDTALKKLEFSSSPDSLSTINDWDDMDDFDTSETSKSFVTPPQSHFVRVSTAQKSKKGKRNFFKAQLYTTNTVKTDLPPPSSESEQIDLTEEQKDDSEWLSSDVICIDDGPIAEVHINEDAQESDSLKTHLEDERDNSEKKKNLEEAELHSTEKVPCIEFDDDDYDTDFVPPSPEEIISASSSSSKCLSTLKDLDTSDRKEDVLSTSKDLLSKPEKMSMQELNPETSTDCDARQISLQQQLIHVMEHICKLIDTIPDDKLKLLDCGNELLQQRNIRRKLLTEVDFNKSDASLLGSLWRYRPDSLDGPMEGDSCPTGNSMKELNFSHLPSNSVSPGDCLLTTTLGKTGFSATRKNLFERPLFNTHLQKSFVSSNWAETPRLGKKNESSYFPGNVLTSTAVKDQNKHTASINDLERETQPSYDIDNFDIDDFDDDDDWEDIMHNLAASKSSTAAYQPIKEGRPIKSVSERLSSAKTDCLPVSSTAQNINFSESIQNYTDKSAQNLASRNLKHERFQSLSFPHTKEMMKIFHKKFGLHNFRTNQLEAINAALLGEDCFILMPTGGGKSLCYQLPACVSPGVTVVISPLRSLIVDQVQKLTSLDIPATYLTGDKTDSEATNIYLQLSKKDPIIKLLYVTPEKICASNRLISTLENLYERKLLARFVIDEAHCVSQWGHDFRQDYKRMNMLRQKFPSVPVMALTATANPRVQKDILTQLKILRPQVFSMSFNRHNLKYYVLPKKPKKVAFDCLEWIRKHHPYDSGIIYCLSRRECDTMADTLQRDGLAALAYHAGLSDSARDEVQQKWINQDGCQVICATIAFGMGIDKPDVRFVIHASLPKSVEGYYQESGRAGRDGEISHCLLFYTYHDVTRLKRLIMMEKDGNHHTRETHFNNLYSMVHYCENITECRRIQLLAYFGENGFNPDFCKKHPDVSCDNCCKTKDYKTRDVTDDVKSIVRFVQEHSSSQGMRNIKHVGPSGRFTMNMLVDIFLESLSSDPEVLLQIDGVTEDKLEKYGAEVISVLQKYSEWTSPAEDSSPGISLSSSRGPGRSAAEELDEEIPVSSHYFASKTRNERKRKKMPASQRSKRRKTASSGSKAKGGSATCRKISSKTKSSSIIGSSSASHTSQATSGANSKLGIMAPPKPINRPFLKPSYAFS

SEQ ID NO: 40 - DNA, 3129 bp, Homo sapiens
atggagcacatctgtaaattaattgatactattcctgatgataaactgaaacttttggattgtgggaacgaactgcttcagcagcggaacataagaaggaaacttctaacggaagtagattttaataaaagtgatgccagtcttcttggctcattgtggagatacaggcctgattcacttgatggccctatggagggtgattcctgccctacagggaattctatgaaggagttaaatttttcacaccttccctcaaattctgtttctcctggggactgtttactgactaccaccctaggaaagacaggattctctgccaccaggaagaatctttttgaaaggcctttattcaatacccatttacagaagtcctttgtaagtagcaactgggctgaaacaccaagactaggaaaaaaaaatgaaagctcttatttcccaggaaatgttctcacaagcactgctgtgaaagatcagaataaacatactgcttcaataaatgacttagaaagagaaacccaaccttcctatgatattgataattttgacatagatgactttgatgatgatgatgactgggaagacataatgcataatttagcagccagcaaatcttccacagctgcctatcaacccatcaaggaaggtcggccaattaaatcagtatcagaaagactttcctcagccaagacagactgtcttccagtgtcatctactgctcaaaatataaacttctcagagtcaattcagaattatactgacaagtcagcacaaaatttagcatccagaaatctgaaacatgagcgtttccaaagtcttagttttcctcatacaaaggaaatgatgaagatttttcataaaaaatttggcctgcataattttagaactaatcagctagaggcgatcaatgctgcactgcttggtgaagactgttttatcctgatgccgactggaggtggtaagagtttgtgttaccagctccctgcctgtgtttctcctggggtcactgttgtcatttctcccttgagatcacttatcgtagatcaagtccaaaagctgacttccttggatattccagctacatatctgacaggtgataagactgactcagaagctacaaatatttacctccagttatcaaaaaaagacccaatcataaaacttctatatgtcactccagaaaagatctgtgcaagtaacagactcatttctactctggagaatctctatgagaggaagctcttggcacgttttgttattgatgaagcacattgtgtcagtcagtggggacatgattttcgtcaagattacaaaagaatgaatatgcttcgccagaagtttccttctgttccggtgatggctcttacggccacagctaatcccagggtacagaaggacatcctgactcagctgaagattctcagacctcaggtgtttagcatgagctttaacagacataatctgaaatactatgtattaccgaaaaagcctaaaaaggtggcatttgattgcctagaatggatcagaaagcaccacccatatgattcagggataatttactgcctctccaggcgagaatgtgacaccatggctgacacgttacagagagatgggctcgctgctcttgcttaccatgctggcctcagtgattctgccagagatgaagtgcagcagaagtggattaatcaggatggctgtcaggttatctgtgctacaattgcatttggaatggggattgacaaaccggacgtgcgatttgtgattcatgcatctctccctaaatctgtggagggttactaccaagaatctggcagagctggaagagatggggaaatatctcactgcctgcttttctatacctatcatgatgtgaccagactgaaaagacttataatgatggaaaaagatggaaaccatcatacaagagaaactcacttcaataatttgtatagcatggtacattactgtgaaaatataacggaatgcaggagaatacagcttttggcctactttggtgaaaatggatttaatcctgatttttgtaagaaacacccagatgtttcttgtgataattgctgtaaaacaaaggattataaaacaagagatgtgactgacgatgtgaaaagtattgtaagatttgttcaagaacatagttcatcacaaggaatgagaaatataaaacatgtaggtccttctggaagatttactatgaatatgctggtcgacattttcttggggagtaagagtgcaaaaatccagtcaggtatatttggaaaaggatctgcttattcacgacacaatgccgaaagactttttaaaaagctgatacttgacaagattttggatgaagacttatatatcaatgccaatgaccaggcgatcgcttatgtgatgctcggaaataaagcccaaactgtactaaatggcaatttaaaggtagactttatggaaacagaaaattccagcagtgtgaaaaaacaaaaagcgttagtagcaaaagtgtctcagagggaagagatggttaaaaaatgtcttggagaacttacagaagtctgcaaatctctggggaaagtttttggtgtccattacttcaatatttttaataccgtcactctcaagaagcttgcagaatctttatcttctgatcctgaggttttgcttcaaattgatggtgttactgaagacaaactggaaaaatatggtgcggaagtgatttcagtattacagaaatactctgaatggacatcgccagctgaagacagttccccagggataagcctgtccagcagcagaggccccggaagaagtgccgctgaggagctcgacgaggaaatacccgtatcttcccactactttgcaagtaaaaccagaaatgaaaggaagaggaaaaagatgccagcctcccaaaggtctaagaggagaaaaactgcttccagtggttccaaggcaaagggggggtctgccacatgtagaaagatatcttccaaaacgaaatcctccagcatcattggatccagttcagcctcacatacttctcaagcgacatcaggagccaatagcaaattggggattatggctccaccgaagcctataaatagaccgtttcttaagccttcatatgcattctcataa

SEQ ID NO: 41 - protein, 1042 aa, Homo sapiens
MEHICKLIDTIPDDKLKLLDCGNELLQQRNIRRKLLTEVDFNKSDASLLGSLWRYRPDSLDGPMEGDSCPTGNSMKELNFSHLPSNSVSPGDCLLTTTLGKTGFSATRKNLFERPLFNTHLQKSFVSSNWAETPRLGKKNESSYFPGNVLTSTAVKDQNKHTASINDLERETQPSYDIDNFDIDDFDDDDDWEDIMHNLAASKSSTAAYQPIKEGRPIKSVSERLSSAKTDCLPVSSTAQNINFSESIQNYTDKSAQNLASRNLKHERFQSLSFPHTKEMMKIFHKKFGLHNFRTNQLEAINAALLGEDCFILMPTGGGKSLCYQLPACVSPGVTVVISPLRSLIVDQVQKLTSLDIPATYLTGDKTDSEATNIYLQLSKKDPIIKLLYVTPEKICASNRLISTLENLYERKLLARFVIDEAHCVSQWGHDFRQDYKRMNMLRQKFPSVPVMALTATANPRVQKDILTQLKILRPQVFSMSFNRHNLKYYVLPKKPKKVAFDCLEWIRKHHPYDSGIIYCLSRRECDTMADTLQRDGLAALAYHAGLSDSARDEVQQKWINQDGCQVICATIAFGMGIDKPDVRFVIHASLPKSVEGYYQESGRAGRDGEISHCLLFYTYHDVTRLKRLIMMEKDGNHHTRETHFNNLYSMVHYCENITECRRIQLLAYFGENGFNPDFCKKHPDVSCDNCCKTKDYKTRDVTDDVKSIVRFVQEHSSSQGMRNIKHVGPSGRFTMNMLVDIFLGSKSAKIQSGIFGKGSAYSRHNAERLFKKLILDKILDEDLYINANDQAIAYVMLGNKAQTVLNGNLKVDFMETENSSSVKKQKALVAKVSQREEMVKKCLGELTEVCKSLGKVFGVHYFNIFNTVTLKKLAESLSSDPEVLLQIDGVTEDKLEKYGAEVISVLQKYSEWTSPAEDSSPGISLSSSRGPGRSAAEELDEEIPVSSHYFASKTRNERKRKKMPASQRSKRRKTASSGSKAKGGSATCRKISSKTKSSSIIGSSSASHTSQATSGANSKLGIMAPPKPINRPFLKPSYAFS

SEQ ID NO: 42 - DNA, 3045 bp, Homo sapiens
atggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaaacaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcacttcatgaaattatatgaagaaaaaaccgggaacgcttggcactccaaaaatttcacgaagtatcccaaaaagttctaccccctggagattgactatggccaggatgaagaggcagtgaagaagctgacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacaatgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactgccatacgtctcagggagacccaataggcttaatcctgttgggagaagttgcccttggaaacatgtatgaactgaagcacgcttcacatatcagcaagttacccaagggcaagcacagtgtcaaaggtttgggcaaaactacccctgatccttcagctaacattagtctggatggtgtagacgttcctcttgggaccgggatttcatctggtgtgaatgacacctctctactatataacgagtacattgtctatgatattgctcaggtaaatctgaagtatctgctgaaactgaaattcaattttaagacctccctgtggtaa

SEQ ID NO: 43 - protein, 1014 aa, Homo sapiens
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

SEQ ID NO: 44 - DNA, 1851 bp, Homo sapiens
atggtcggccaactgagcgagggggccattgcggccatcatgcagaagggggatacaaacataaagcccatcctccaagtcatcaacatccgtcccattactacggggaatagtccgccgcgttatcgactgctcatgagtgatggattgaacactctatcctctttcatgttggcgacacagttgaaccctctcgtggaggaagaacaattgtccagcaactgtgtatgccagattcacagatttattgtgaacactctgaaagacggaaggagagtagttatcttgatggaattagaagttttgaagtcagctgaagcagttggagtgaagattggcaatccagtgccctataatgaaggactcgggcagccgcaagtagctcctccagcgccagcagccagcccagcagcaagcagcaggccccagccgcagaatggaagctcgggaatgggttctactgtttctaaggcttatggtgcttcaaagacatttggaaaagctgcaggtcccagcctgtcacacacttctgggggaacacagtccaaagtggtgcccattgccagcctcactccttaccagtccaagtggaccatttgtgctcgtgttaccaacaaaagtcagatccgtacctggagcaactcccgaggggaagggaagcttttctccctagaactggttgacgaaagtggtgaaatccgagctacagctttcaatgagcaagtggacaagttctttcctcttattgaagtgaacaaggtgtattatttctcgaaaggcaccctgaagattgctaacaagcagttcacagctgttaaaaatgactacgagatgaccttcaataacgagacttccgtcatgccctgtgaggacgaccatcatttacctacggttcagtttgatttcacggggattgatgacctcgagaacaagtcgaaagactcacttgtagacatcatcgggatctgcaagagctatgaagacgccactaaaatcacagtgaggtctaacaacagagaagttgccaagaggaatatctacttgatggacacatccgggaaggtggtgactgctacactgtggggggaagatgctgataaatttgatggttctagacagcccgtgttggctatcaaaggagcccgagtctctgatttcggtggacggagcctctccgtgctgtcttcaagcactatcattgcgaatcctgacatcccagaggcctataagcttcgtggatggtttgacgcagaaggacaagccttagatggtgtttccatctctgatctaaagagcggcggagtcggagggagtaacaccaactggaaaaccttgtatgaggtcaaatccgagaacctgggccaaggcgacaagccggactactttagttctgtggccacagtggtgtatcttcgcaaagagaactgcatgtaccaagcctgcccgactcaggactgcaataagaaagtgattgatcaacagaatggattgtaccgctgtgagaagtgcgacaccgaatttcccaatttcaagtaccgcatgatcctgtcagtaaatattgcagattttcaagagaatcagtgggtgacttgtttccaggagtctgctgaagctatccttggacaaaatgctgcttatcttggggaattaaaagacaagaatgaacaggcatttgaagaagttttccagaatgccaacttccgatctttcatattcagagtcagggtcaaagtggagacctacaacgacgagtctcgaattaaggccactgtgatggacgtgaagcccgtggactacagagagtatggccgaaggctggtcatgagcatcaggagaagtgcattgatgtga

SEQ ID NO: 45 - protein, 616 aa, Homo sapiens
MVGQLSEGAIAAIMQKGDTNIKPILQVINIRPITTGNSPPRYRLLMSDGLNTLSSFMLATQLNPLVEEEQLSSNCVCQIHRFIVNTLKDGRRVVILMELEVLKSAEAVGVKIGNPVPYNEGLGQPQVAPPAPAASPAASSRPQPQNGSSGMGSTVSKAYGASKTFGKAAGPSLSHTSGGTQSKVVPIASLTPYQSKWTICARVTNKSQIRTWSNSRGEGKLFSLELVDESGEIRATAFNEQVDKFFPLIEVNKVYYFSKGTLKIANKQFTAVKNDYEMTFNNETSVMPCEDDHHLPTVQFDFTGIDDLENKSKDSLVDIIGICKSYEDATKITVRSNNREVAKRNIYLMDTSGKVVTATLWGEDADKFDGSRQPVLAIKGARVSDFGGRSLSVLSSSTIIANPDIPEAYKLRGWFDAEGQALDGVSISDLKSGGVGGSNTNWKTLYEVKSENLGQGDKPDYFSSVATVVYLRKENCMYQACPTQDCNKKVIDQQNGLYRCEKCDTEFPNFKYRMILSVNIADFQENQWVTCFQESAEAILGQNAAYLGELKDKNEQAFEEVFQNANFRSFIFRVRVKVETYNDESRIKATVMDVKPVDYREYGRRLVMSIRRSALM

SEQ ID NO: 46 - DNA, 1812 bp, Homo sapiens
atgcagaagggggatacaaacataaagcccatcctccaagtcatcaacatccgtcccattactacggggaatagtccgccgcgttatcgactgctcatgagtgatggattgaacactctatcctctttcatgttggcgacacagttgaaccctctcgtggaggaagaacaattgtccagcaactgtgtatgccagattcacagatttattgtgaacactctgaaagacggaaggagagtagttatcttgatggaattagaagttttgaagtcagctgaagcagttggagtgaagattggcaatccagtgccctataatgaaggactcgggcagccgcaagtagctcctccagcgccagcagccagcccagcagcaagcagcaggccccagccgcagaatggaagctcgggaatgggttctactgtttctaaggcttatggtgcttcaaagacatttggaaaagctgcaggtcccagcctgtcacacacttctgggggaacacagtccaaagtggtgcccattgccagcctcactccttaccagtccaagtggaccatttgtgctcgtgttaccaacaaaagtcagatccgtacctggagcaactcccgaggggaagggaagcttttctccctagaactggttgacgaaagtggtgaaatccgagctacagctttcaatgagcaagtggacaagttctttcctcttattgaagtgaacaaggtgtattatttctcgaaaggcaccctgaagattgctaacaagcagttcacagctgttaaaaatgactacgagatgaccttcaataacgagacttccgtcatgccctgtgaggacgaccatcatttacctacggttcagtttgatttcacggggattgatgacctcgagaacaagtcgaaagactcacttgtagacatcatcgggatctgcaagagctatgaagacgccactaaaatcacagtgaggtctaacaacagagaagttgccaagaggaatatctacttgatggacacatccgggaaggtggtgactgctacactgtggggggaagatgctgataaatttgatggttctagacagcccgtgttggctatcaaaggagcccgagtctctgatttcggtggacggagcctctccgtgctgtcttcaagcactatcattgcgaatcctgacatcccagaggcctataagcttcgtggatggtttgacgcagaaggacaagccttagatggtgtttccatctctgatctaaagagcggcggagtcggagggagtaacaccaactggaaaaccttgtatgaggtcaaatccgagaacctgggccaaggcgacaagccggactactttagttctgtggccacagtggtgtatcttcgcaaagagaactgcatgtaccaagcctgcccgactcaggactgcaataagaaagtgattgatcaacagaatggattgtaccgctgtgagaagtgcgacaccgaatttcccaatttcaagtaccgcatgatcctgtcagtaaatattgcagattttcaagagaatcagtgggtgacttgtttccaggagtctgctgaagctatccttggacaaaatgctgcttatcttggggaattaaaagacaagaatgaacaggcatttgaagaagttttccagaatgccaacttccgatctttcatattcagagtcagggtcaaagtggagacctacaacgacgagtctcgaattaaggccactgtgatggacgtgaagcccgtggactacagagagtatggccgaaggctggtcatgagcatcaggagaagtgcattgatgtga

SEQ ID NO: 47 - protein, 603 aa, Homo sapiens
MQKGDTNIKPILQVINIRPITTGNSPPRYRLLMSDGLNTLSSFMLATQLNPLVEEEQLSSNCVCQIHRFIVNTLKDGRRVVILMELEVLKSAEAVGVKIGNPVPYNEGLGQPQVAPPAPAASPAASSRPQPQNGSSGMGSTVSKAYGASKTFGKAAGPSLSHTSGGTQSKVVPIASLTPYQSKWTICARVTNKSQIRTWSNSRGEGKLFSLELVDESGEIRATAFNEQVDKFFPLIEVNKVYYFSKGTLKIANKQFTAVKNDYEMTFNNETSVMPCEDDHHLPTVQFDFTGIDDLENKSKDSLVDIIGICKSYEDATKITVRSNNREVAKRNIYLMDTSGKVVTATLWGEDADKFDGSRQPVLAIKGARVSDFGGRSLSVLSSSTIIANPDIPEAYKLRGWFDAEGQALDGVSISDLKSGGVGGSNTNWKTLYEVKSENLGQGDKPDYFSSVATVVYLRKENCMYQACPTQDCNKKVIDQQNGLYRCEKCDTEFPNFKYRMILSVNIADFQENQWVTCFQESAEAILGQNAAYLGELKDKNEQAFEEVFQNANFRSFIFRVRVKVETYNDESRIKATVMDVKPVDYREYGRRLVMSIRRSALM

SEQ ID NO: 48 - DNA, 1674 bp, Homo sapiens
atggtcggccaactgagcgagggggccattgcggccatcatgcagaagggggatacaaacataaagcccatcctccaagtcatcaacatccgtcccattactacggggaatagtccgccgcgttatcgactgctcatgagtgatggattgaacactctatcctctttcatgttggcgacacagttgaaccctctcgtggaggaagaacaattgtccagcaactgtgtatgccagattcacagatttattgtgaacactctgaaagacggaaggagagtagttatcttgatggaattagaagttttgaagtcagctgaagcagttggagtgaagattggcaatccagtgccctataatgaaggactcgggcagccgcaagtagctcctccagcgccagcagccagcccagcagcaagcagcaggccccagccgcagaatggaagctcgggaatgggttctactgtttctaaggcttatggtgcttcaaagacatttggaaaagctgcaggtcccagcctgtcacacacttctgggggaacacagtccaaagtggtgcccattgccagcctcactccttaccagtccaagtggaccatttgtgctcgtgttaccaacaaaagtcagatccgtacctggagcaactcccgaggggaagggaagcttttctccctagaactggttgacgaaagtggtgaaatccgagctacagctttcaatgagcaagtggacaagttctttcctcttattgaagtgaacaaggtgtattatttctcgaaaggcaccctgaagattgctaacaagcagttcacagctgttaaaaatgactacgagatgaccttcaataacgagacttccgtcatgccctgtgaggacgaccatcatttacctacggttcagtttgatttcacggggattgatgacctcgagaacaagtcgaaagactcacttgtagacatcatcgggatctgcaagagctatgaagacgccactaaaatcacagtgaggtctaacaacagagaagttgccaagaggaatatctacttgatggacacatccgggaaggtggtgactgctacactgtggggggaagatgctgataaatttgatggttctagacagcccgtgttggctatcaaaggagcccgagtctctgatttcggtggacggagcctctccgtgctgtcttcaagcactatcattgcgaatcctgacatcccagaggcctataagcttcgtggatggtttgacgcagaaggacaagccttagatggtgtttccatctctgatctaaagagcggcggagtcggagggagtaacaccaactggaaaaccttgtatgaggtcaaatccgagaacctgggccaaggcgacaaggtaaatattgcagattttcaagagaatcagtgggtgacttgtttccaggagtctgctgaagctatccttggacaaaatgctgcttatcttggggaattaaaagacaagaatgaacaggcatttgaagaagttttccagaatgccaacttccgatctttcatattcagagtcagggtcaaagtggagacctacaacgacgagtctcgaattaaggccactgtgatggacgtgaagcccgtggactacagagagtatggccgaaggctggtcatgagcatcaggagaagtgcattgatgtga

SEQ ID NO: 49 - protein, 557 aa, Homo sapiens
MVGQLSEGAIAAIMQKGDTNIKPILQVINIRPITTGNSPPRYRLLMSDGLNTLSSFMLATQLNPLVEEEQLSSNCVCQIHRFIVNTLKDGRRVVILMELEVLKSAEAVGVKIGNPVPYNEGLGQPQVAPPAPAASPAASSRPQPQNGSSGMGSTVSKAYGASKTFGKAAGPSLSHTSGGTQSKVVPIASLTPYQSKWTICARVTNKSQIRTWSNSRGEGKLFSLELVDESGEIRATAFNEQVDKFFPLIEVNKVYYFSKGTLKIANKQFTAVKNDYEMTFNNETSVMPCEDDHHLPTVQFDFTGIDDLENKSKDSLVDIIGICKSYEDATKITVRSNNREVAKRNIYLMDTSGKVVTATLWGEDADKFDGSRQPVLAIKGARVSDFGGRSLSVLSSSTIIANPDIPEAYKLRGWFDAEGQALDGVSISDLKSGGVGGSNTNWKTLYEVKSENLGQGDKVNIADFQENQWVTCFQESAEAILGQNAAYLGELKDKNEQAFEEVFQNANFRSFIFRVRVKVETYNDESRIKATVMDVKPVDYREYGRRLVMSIRRSALM

SEQ ID NO: 50 - DNA, 1020 bp, Homo sapiens
atggcaatgcagatgcagcttgaagcaaatgcagatacttcagtggaagaagaaagctttggcccacaacccatttcacggttagagcagtgtggcataaatgccaacgatgtgaagaaattggaagaagctggattccatactgtggaggctgttgcctatgcgccaaagaaggagctaataaatattaagggaattagtgaagccaaagctgataaaattctggctgaggcagctaaattagttccaatgggtttcaccactgcaactgaattccaccaaaggcggtcagagatcatacagattactactggctccaaagagcttgacaaactacttcaaggtggaattgagactggatctatcacagaaatgtttggagaattccgaactgggaagacccagatctgtcatacgctagctgtcacctgccagcttcccattgaccggggtggaggtgaaggaaaggccatgtacattgacactgagggtacctttaggccagaacggctgctggcagtggctgagaggtatggtctctctggcagtgatgtcctggataatgtagcatatgctcgagcgttcaacacagaccaccagacccagctcctttatcaagcatcagccatgatggtagaatctaggtatgcactgcttattgtagacagtgccaccgccctttacagaacagactactcgggtcgaggtgagctttcagccaggcagatgcacttggccaggtttctgcggatgcttctgcgactcgctgatgagtttggtgtagcagtggtaatcactaatcaggtggtagctcaagtggatggagcagcgatgtttgctgctgatcccaaaaaacctattggaggaaatatcatcgcccatgcatcaacaaccagattgtatctgaggaaaggaagaggggaaaccagaatctgcaaaatctacgactctccctgtcttcctgaagctgaagctatgttcgccattaatgcagatggagtgggagatgccaaagactga

SEQ ID NO: 51 - protein, 339 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 52 - DNA, 1023 bp, Homo sapiens
atggcaatgcagatgcagcttgaagcaaatgcagatacttcagtggaagaagaaagctttggcccacaacccatttcacggttagagcagtgtggcataaatgccaacgatgtgaagaaattggaagaagctggattccatactgtggaggctgttgcctatgcgccaaagaaggagctaataaatattaagggaattagtgaagccaaagctgataaaattctgacggagtctcgctctgttgccaggctggagtgcaatagcgtgatcttggtctactgcaccctccgcctctcaggttcaagtgattctcctgcctcagcctcccgagtagttgggactacaggtggaattgagactggatctatcacagaaatgtttggagaattccgaactgggaagacccagatctgtcatacgctagctgtcacctgccagcttcccattgaccggggtggaggtgaaggaaaggccatgtacattgacactgagggtacctttaggccagaacggctgctggcagtggctgagaggtatggtctctctggcagtgatgtcctggataatgtagcatatgctcgagcgttcaacacagaccaccagacccagctcctttatcaagcatcagccatgatggtagaatctaggtatgcactgcttattgtagacagtgccaccgccctttacagaacagactactcgggtcgaggtgagctttcagccaggcagatgcacttggccaggtttctgcggatgcttctgcgactcgctgatgagtttggtgtagcagtggtaatcactaatcaggtggtagctcaagtggatggagcagcgatgtttgctgctgatcccaaaaaacctattggaggaaatatcatcgcccatgcatcaacaaccagattgtatctgaggaaaggaagaggggaaaccagaatctgcaaaatctacgactctccctgtcttcctgaagctgaagctatgttcgccattaatgcagatggagtgggagatgccaaagactga

SEQ ID NO: 53 - protein, 340 aa, Homo sapiens
MAMQMQLEANADTSVEEESFGPQPISRLEQCGINANDVKKLEEAGFHTVEAVAYAPKKELINIKGISEAKADKILTESRSVARLECNSVILVYCTLRLSGSSDSPASASRVVGTTGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 54 - DNA, 843 bp, Homo sapiens
atggcaatgcagatgcagcttgaagcaaatgcagatacttcagtggaagaagaaagctttggcccacaacccatttcacggttagagcagtgtggcataaatgccaacgatgtgaagaaattggaagaagctggattccatactgtggaggctgttgcctatgcgccaaagaaggagctaataaatattaagggaattagtgaagccaaagctgataaaattctggctgaggcagctaaattagttccaatgggtttcaccactgcaactgaattccaccaaaggcggtcagagatcatacagattactactggctccaaagagcttgacaaactacttcaaggtggaattgagactggatctatcacagaaatgtttggagaattccgaactgggaagacccagatctgtcatacgctagctgtcacctgccagcttcccattgaccggggtggaggtgaaggaaaggccatgtacattgacactgagggtacctttaggccagaacggctgctggcagtggctgagaggtatggtctctctggcagtgatgtcctggataatgtagcatatgctcgagcgttcaacacagaccaccagacccagctcctttatcaagcatcagccatgatggtagaatctaggtatgcactgcttattgtagacagtgccaccgccctttacagaacagactactcgggtcgaggtgagctttcagccaggcagatgcacttggccaggtttctgcggatgcttctgcgactcgctgatgagattgtatctgaggaaaggaagaggggaaaccagaatctgcaaaatctacgactctccctgtcttcctga

SEQ ID NO: 55 - protein, 280 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEIVSEERKRGNQNLQNLRLSLSS

SEQ ID NO: 56 - DNA, 1659 bp, Homo sapiens
atggcggccccggtccgcctgggccggaagcgcccgctgcctgcctgtcccaacccgctcttcgttcgctggctgaccgagtggcgggacgaggcgacccgcagcaggcgccgcacgcgcttcgtatttcagaaggcgctgcgttccctccgacggtacccactgccgctgcgcagcgggaaggaagctaagatcctacagcacttcggagacgggctctgccggatgctggacgagcggctgcagcggcaccgaacatcgggcggtgaccatgccccggactcaccatctggagagaacagtccagccccgcaggggcgacttgcggaagtccaggactcttccatgccagttcctgcccagcccaaagcgggaggctctggcagctactggccagctcggcactcaggagcccgagtgatactgctggtgctctaccgggagcacctgaatcctaatggtcaccacttcttaaccaaggaggagctgctgcagaggtgtgctcagaagtcccccagggtagcccctgggagtgctcgaccctggccagccctccgctccctccttcacaggaacctggtcctcaggacacaccagccagccaggtactcattgaccccagagggcctggagctggcccagaagttggccgagtcagaaggcctgagcttgctgaatgtgggcatcgggcccaaggagccccctggggaggagacagcagtgccaggagcagcttcagcagagcttgccagtgaagcaggggtccagcagcagccactggagctgaggcctggagagtacagggtgctgttgtgtgtggacattggcgagacccgggggggcgggcacaggccggagctgctccgagagctacagcggctgcacgtgacccacacggtgcgcaagctgcacgttggagattttgtgtgggtggcccaggagaccaatcctagagacccagcagcaaaccctggggagttggtactggatcacattgtggagcgcaagcgactggatgacctttgcagcagcatcatcgacggccgcttccgggagcagaagttccggctgaagcgctgtggtctggagcgccgggtatacctggtggaagagcatggttccgtccacaacctcagccttcctgagagcacactgctgcaggctgtcaccaacactcaggtcattgatggcttttttgtgaagcgcacagcagacattaaggagtcagccgcctacctggccctcttgacgcggggcctgcagagactctaccagggccacaccctacgcagccgcccctggggaacccctgggaaccctgaatcaggggccatgacctctccaaaccctctctgctcactcctcaccttcagtgacttcaacgcaggagccatcaagaataaggcccagtcggtgcgagaagtgtttgcccggcagctgatgcaggtgcgcggagtgagtggggagaaggcagcagccctggtggatcgatacagcacccctgccagcctcctggccgcctatgatgcctgtgccacccccaaggaacaagagacactgctgagcaccattaagtgtgggcgtctacagaggaatctggggcctgctctgagcaggaccttatcccagctctactgcagctacggccccttgacctga

SEQ ID NO: 57 - protein, 552 aa, Homo sapiens
MAAPVRLGRKRPLPACPNPLFVRWLTEWRDEATRSRRRTRFVFQKALRSLRRYPLPLRSGKEAKILQHFGDGLCRMLDERLQRHRTSGGDHAPDSPSGENSPAPQGRLAEVQDSSMPVPAQPKAGGSGSYWPARHSGARVILLVLYREHLNPNGHHFLTKEELLQRCAQKSPRVAPGSARPWPALRSLLHRNLVLRTHQPARYSLTPEGLELAQKLAESEGLSLLNVGIGPKEPPGEETAVPGAASAELASEAGVQQQPLELRPGEYRVLLCVDIGETRGGGHRPELLRELQRLHVTHTVRKLHVGDFVWVAQETNPRDPAANPGELVLDHIVERKRLDDLCSSIIDGRFREQKFRLKRCGLERRVYLVEEHGSVHNLSLPESTLLQAVTNTQVIDGFFVKRTADIKESAAYLALLTRGLQRLYQGHTLRSRPWGTPGNPESGAMTSPNPLCSLLTFSDFNAGAIKNKAQSVREVFARQLMQVRGVSGEKAAALVDRYSTPASLLAAYDACATPKEQETLLSTIKCGRLQRNLGPALSRTLSQLYCSYGPLT

SEQ ID NO: 58 - DNA, 1656 bp, Homo sapiens
atggcggccccggtccgcctgggccggaagcgcccgctgcctgcctgtcccaacccgctcttcgttcgctggctgaccgagtggcgggacgaggcgacccgcagcaggcgccgcacgcgcttcgtatttcagaaggcgctgcgttccctccgacggtacccactgccgctgcgcagcgggaaggaagctaagatcctacagcacttcggagacgggctctgccggatgctggacgagcggctgcagcggcaccgaacatcgggcggtgaccatgccccggactcaccatctggagagaacagtccagccccgcaggggcgacttgcggaagtccaggactcttccatgccagttcctgcccagcccaaagcgggaggctctggcagctactggccagctcggcactcaggagcccgagtgatactgctggtgctctaccgggagcacctgaatcctaatggtcaccacttcttaaccaaggaggagctgctgcagaggtgtgctcagaagtcccccagggtagcccctgggagtgctcgaccctggccagccctccgctccctccttcacaggaacctggtcctcaggacacaccagccagccaggtactcattgaccccagagggcctggagctggcccagaagttggccgagtcagaaggcctgagcttgctgaatgtgggcatcgggcccaaggagccccctggggaggagacagcagtgccaggagcagcttcagcagagcttgccagtgaagcaggggtccagcagcagccactggagctgaggcctggagagtacagggtgctgttgtgtgtggacattggcgagacccgggggggcgggcacaggccggagctgctccgagagctacagcggctgcacgtgacccacacggtgcgcaagctgcacgttggagattttgtgtgggtggcccaggagaccaatcctagagacccagcaaaccctggggagttggtactggatcacattgtggagcgcaagcgactggatgacctttgcagcagcatcatcgacggccgcttccgggagcagaagttccggctgaagcgctgtggtctggagcgccgggtatacctggtggaagagcatggttccgtccacaacctcagccttcctgagagcacactgctgcaggctgtcaccaacactcaggtcattgatggcttttttgtgaagcgcacagcagacattaaggagtcagccgcctacctggccctcttgacgcggggcctgcagagactctaccagggccacaccctacgcagccgcccctggggaacccctgggaaccctgaatcaggggccatgacctctccaaaccctctctgctcactcctcaccttcagtgacttcaacgcaggagccatcaagaataaggcccagtcggtgcgagaagtgtttgcccggcagctgatgcaggtgcgcggagtgagtggggagaaggcagcagccctggtggatcgatacagcacccctgccagcctcctggccgcctatgatgcctgtgccacccccaaggaacaagagacactgctgagcaccattaagtgtgggcgtctacagaggaatctggggcctgctctgagcaggaccttatcccagctctactgcagctacggccccttgacctga

SEQ ID NO: 59 - protein, 551 aa, Homo sapiens
MAAPVRLGRKRPLPACPNPLFVRWLTEWRDEATRSRRRTRFVFQKALRSLRRYPLPLRSGKEAKILQHFGDGLCRMLDERLQRHRTSGGDHAPDSPSGENSPAPQGRLAEVQDSSMPVPAQPKAGGSGSYWPARHSGARVILLVLYREHLNPNGHHFLTKEELLQRCAQKSPRVAPGSARPWPALRSLLHRNLVLRTHQPARYSLTPEGLELAQKLAESEGLSLLNVGIGPKEPPGEETAVPGAASAELASEAGVQQQPLELRPGEYRVLLCVDIGETRGGGHRPELLRELQRLHVTHTVRKLHVGDFVWVAQETNPRDPANPGELVLDHIVERKRLDDLCSSIIDGRFREQKFRLKRCGLERRVYLVEEHGSVHNLSLPESTLLQAVTNTQVIDGFFVKRTADIKESAAYLALLTRGLQRLYQGHTLRSRPWGTPGNPESGAMTSPNPLCSLLTFSDFNAGAIKNKAQSVREVFARQLMQVRGVSGEKAAALVDRYSTPASLLAAYDACATPKEQETLLSTIKCGRLQRNLGPALSRTLSQLYCSYGPLT

SEQ ID NO: 60 - DNA, 2358 bp, Homo sapiens
atgggaaaaaaatacaagaacattgttctactaaaaggattagaggtcatcaatgattatcattttagaatggttaagtccttactgagcaacgatttaaaacttaatttaaaaatgagagaagagtatgacaaaattcagattgctgacttgatggaagaaaagttccgaggtgatgctggtttgggcaaactaataaaaattttcgaagatataccaacgcttgaagacctggctgaaactcttaaaaaagaaaagttaaaagtaaaaggaccagccctatcaagaaagaggaagaaggaagtggatgctacttcacctgcaccctccacaagcagcactgtcaaaactgaaggagcagaggcaactcctggagctcagaaaagaaaaaaatcaaccaaagaaaaggctggacccaaagggagtaaggtgtccgaggaacagactcagcctccctctcctgcaggagccggcatgtccacagccatgggccgttccccatctcccaagacctcattgtcagctccacccaacagttcttcaactgagaacccgaaaacagtggccaaatgtcaggtaactcccagaagaaatgttctccaaaaacgcccagtgatagtgaaggtactgagtacaacaaagccatttgaatatgagaccccagaaatggagaaaaaaataatgtttcatgctacagtggctacacagacacagttcttccatgtgaaggttttaaacaccagcttgaaggagaaattcaatggaaagaaaatcatcatcatatcagattatttggaatatgatagtctcctagaggtcaatgaagaatctactgtatctgaagctggtcctaaccaaacgtttgaggttccaaataaaatcatcaacagagcaaaggaaactctgaagattgatattcttcacaaacaagcttcaggaaatattgtatatggggtatttatgctacataagaaaacagtaaatcagaagaccacaatctacgaaattcaggatgatagaggaaaaatggatgtagtggggacaggacaatgtcacaatatcccctgtgaagaaggagataagctccaacttttctgctttcgacttagaaaaaagaaccagatgtcaaaactgatttcagaaatgcatagttttatccagataaagaaaaaaacaaacccgagaaacaatgaccccaagagcatgaagctaccccaggaacagcgtcagcttccatatccttcagaggccagcacaaccttccctgagagccatcttcggactcctcagatgccaccaacaactccatccagcagtttcttcaccaagaaaagtgaagacacaatctccaaaatgaatgacttcatgaggatgcagatactgaaggaagggagtcattttccaggaccgttcatgaccagcataggcccagctgagagccatccccacactcctcagatgcctccatcaacaccaagcagcagtttcttaaccacgaaaagtgaagacacaatctccaaaatgaatgacttcatgaggatgcagatactgaaggaagggagtcattttccaggaccgttcatgaccagcataggcccagctgagagccatccccacactcctcagatgcctccatcaacaccaagcagcagtttcttaaccacgttgaaaccaagactgaagactgaacctgaagaagtttccatagaagacagtgcccagagtgacctcaaagaagtgatggtgctgaacgcaacagaatcatttgtatatgagcccaaagagcagaagaaaatgtttcatgccacagtggcaactgagaatgaagtcttccgagtgaaggtttttaatattgacctaaaggagaagttcaccccaaagaagatcattgccatagcaaattatgtttgccgcaatgggttcctggaggtatatcctttcacacttgtggctgatgtgaatgctgaccgaaacatggagatcccaaaaggattgattagaagtgccagcgtaactcctaaaatcaatcagctttgctcacaaactaaaggaagttttgtgaatggggtgtttgaggtacataagaaaaatgtaaggggtgaattcacttattatgaaatacaagataatacagggaagatggaagtggtggtgcatggacgactgaccacaatcaactgtgaggaaggagataaactgaaactcacctgctttgaattggcaccgaaaagtgggaataccggggagttgagatctgtaattcatagtcacatcaaggtcatcaagaccaggaaaaacaagaaagacatactcaatcctgattcaagtatggaaacttcaccagactttttcttctaa

SEQ ID NO: 61 - protein, 785 aa, Homo sapiens
MGKKYKNIVLLKGLEVINDYHFRMVKSLLSNDLKLNLKMREEYDKIQIADLMEEKFRGDAGLGKLIKIFEDIPTLEDLAETLKKEKLKVKGPALSRKRKKEVDATSPAPSTSSTVKTEGAEATPGAQKRKKSTKEKAGPKGSKVSEEQTQPPSPAGAGMSTAMGRSPSPKTSLSAPPNSSSTENPKTVAKCQVTPRRNVLQKRPVIVKVLSTTKPFEYETPEMEKKIMFHATVATQTQFFHVKVLNTSLKEKFNGKKIIIISDYLEYDSLLEVNEESTVSEAGPNQTFEVPNKIINRAKETLKIDILHKQASGNIVYGVFMLHKKTVNQKTTIYEIQDDRGKMDVVGTGQCHNIPCEEGDKLQLFCFRLRKKNQMSKLISEMHSFIQIKKKTNPRNNDPKSMKLPQEQRQLPYPSEASTTFPESHLRTPQMPPTTPSSSFFTKKSEDTISKMNDFMRMQILKEGSHFPGPFMTSIGPAESHPHTPQMPPSTPSSSFLTTKSEDTISKMNDFMRMQILKEGSHFPGPFMTSIGPAESHPHTPQMPPSTPSSSFLTTLKPRLKTEPEEVSIEDSAQSDLKEVMVLNATESFVYEPKEQKKMFHATVATENEVFRVKVFNIDLKEKFTPKKIIAIANYVCRNGFLEVYPFTLVADVNADRNMEIPKGLIRSASVTPKINQLCSQTKGSFVNGVFEVHKKNVRGEFTYYEIQDNTGKMEVVVHGRLTTINCEEGDKLKLTCFELAPKSGNTGELRSVIHSHIKVIKTRKNKKDILNPDSSMETSPDFFF

SEQ ID NO: 62 - DNA, 2190 bp, Homo sapiens
atgggaaaaaaatacaagaacattgttctactaaaaggattagaggtcatcaatgattatcattttagaatggttaagtccttactgagcaacgatttaaaacttaatttaaaaatgagagaagagtatgacaaaattcagattgctgacttgatggaagaaaagttccgaggtgatgctggtttgggcaaactaataaaaattttcgaagatataccaacgcttgaagacctggctgaaactcttaaaaaagaaaagttaaaagtaaaaggaccagccctatcaagaaagaggaagaaggaagtggatgctacttcacctgcaccctccacaagcagcactgtcaaaactgaaggagcagaggcaactcctggagctcagaaaagaaaaaaatcaaccaaagaaaaggctggacccaaagggagtaaggtgtccgaggaacagactcagcctccctctcctgcaggagccggcatgtccacagccatgggccgttccccatctcccaagacctcattgtcagctccacccaacagttcttcaactgagaacccgaaaacagtggccaaatgtcaggtaactcccagaagaaatgttctccaaaaacgcccagtgatagtgaaggtactgagtacaacaaagccatttgaatatgagaccccagaaatggagaaaaaaataatgtttcatgctacagtggctacacagacacagttcttccatgtgaaggttttaaacaccagcttgaaggagaaattcaatggaaagaaaatcatcatcatatcagattatttggaatatgatagtctcctagaggtcaatgaagaatctactgtatctgaagctggtcctaaccaaacgtttgaggttccaaataaaatcatcaacagagcaaaggaaactctgaagattgatattcttcacaaacaagcttcaggaaatattgtatatggggtatttatgctacataagaaaacagtaaatcagaagaccacaatctacgaaattcaggatgatagaggaaaaatggatgtagtggggacaggacaatgtcacaatatcccctgtgaagaaggagataagctccaacttttctgctttcgacttagaaaaaagaaccagatgtcaaaactgatttcagaaatgcatagttttatccagataaagaaaaaaacaaacccgagaaacaatgaccccaagagcatgaagctaccccaggaacagcgtcagcttccatatccttcagaggccagcacaaccttccctgagagccatcttcggactcctcagatgccaccaacaactccatccagcagtttcttcaccaagaaaagtgaagacacaatctccaaaatgaatgacttcatgaggatgcagatactgaaggaagggagtcattttccaggaccgttcatgaccagcataggcccagctgagagccatccccacactcctcagatgcctccatcaacaccaagcagcagtttcttaaccacgttgaaaccaagactgaagactgaacctgaagaagtttccatagaagacagtgcccagagtgacctcaaagaagtgatggtgctgaacgcaacagaatcatttgtatatgagcccaaagagcagaagaaaatgtttcatgccacagtggcaactgagaatgaagtcttccgagtgaaggtttttaatattgacctaaaggagaagttcaccccaaagaagatcattgccatagcaaattatgtttgccgcaatgggttcctggaggtatatcctttcacacttgtggctgatgtgaatgctgaccgaaacatggagatcccaaaaggattgattagaagtgccagcgtaactcctaaaatcaatcagctttgctcacaaactaaaggaagttttgtgaatggggtgtttgaggtacataagaaaaatgtaaggggtgaattcacttattatgaaatacaagataatacagggaagatggaagtggtggtgcatggacgactgaccacaatcaactgtgaggaaggagataaactgaaactcacctgctttgaattggcaccgaaaagtgggaataccggggagttgagatctgtaattcatagtcacatcaaggtcatcaagaccaggaaaaacaagaaagacatactcaatcctgattcaagtatggaaacttcaccagactttttcttctaa

SEQ ID NO: 63 - protein, 729 aa, Homo sapiens
MGKKYKNIVLLKGLEVINDYHFRMVKSLLSNDLKLNLKMREEYDKIQIADLMEEKFRGDAGLGKLIKIFEDIPTLEDLAETLKKEKLKVKGPALSRKRKKEVDATSPAPSTSSTVKTEGAEATPGAQKRKKSTKEKAGPKGSKVSEEQTQPPSPAGAGMSTAMGRSPSPKTSLSAPPNSSSTENPKTVAKCQVTPRRNVLQKRPVIVKVLSTTKPFEYETPEMEKKIMFHATVATQTQFFHVKVLNTSLKEKFNGKKIIIISDYLEYDSLLEVNEESTVSEAGPNQTFEVPNKIINRAKETLKIDILHKQASGNIVYGVFMLHKKTVNQKTTIYEIQDDRGKMDVVGTGQCHNIPCEEGDKLQLFCFRLRKKNQMSKLISEMHSFIQIKKKTNPRNNDPKSMKLPQEQRQLPYPSEASTTFPESHLRTPQMPPTTPSSSFFTKKSEDTISKMNDFMRMQILKEGSHFPGPFMTSIGPAESHPHTPQMPPSTPSSSFLTTLKPRLKTEPEEVSIEDSAQSDLKEVMVLNATESFVYEPKEQKKMFHATVATENEVFRVKVFNIDLKEKFTPKKIIAIANYVCRNGFLEVYPFTLVADVNADRNMEIPKGLIRSASVTPKINQLCSQTKGSFVNGVFEVHKKNVRGEFTYYEIQDNTGKMEVVVHGRLTTINCEEGDKLKLTCFELAPKSGNTGELRSVIHSHIKVIKTRKNKKDILNPDSSMETSPDFFF

SEQ ID NO: 64 - DNA, 2190 bp, Homo sapiens
atgggaaaaaaatacaagaacattgttctactaaaaggattagaggtcatcaatgattatcattttagaatggttaagtccttactgagcaacgatttaaaacttaatttaaaaatgagagaagagtatgacaaaattcagattgctgacttgatggaagaaaagttccgaggtgatgctggtttgggcaaactaataaaaattttcgaagatataccaacgcttgaagacctggctgaaactcttaaaaaagaaaagttaaaagtaaaaggaccagccctatcaagaaagaggaagaaggaagtggatgctacttcacctgcaccctccacaagcagcactgtcaaaactgaaggagcagaggcaactcctggagctcagaacccgaaaacagtggccaaatgtcaggtaactcccagaagaaatgttctccaaaaacgcccagtgatagtgaaggtactgagtacaacaaagccatttgaatatgagaccccagaaatggagaaaaaaataatgtttcatgctacagtggctacacagacacagttcttccatgtgaaggttttaaacaccagcttgaaggagaaattcaatggaaagaaaatcatcatcatatcagattatttggaatatgatagtctcctagaggtcaatgaagaatctactgtatctgaagctggtcctaaccaaacgtttgaggttccaaataaaatcatcaacagagcaaaggaaactctgaagattgatattcttcacaaacaagcttcaggaaatattgtatatggggtatttatgctacataagaaaacagtaaatcagaagaccacaatctacgaaattcaggatgatagaggaaaaatggatgtagtggggacaggacaatgtcacaatatcccctgtgaagaaggagataagctccaacttttctgctttcgacttagaaaaaagaaccagatgtcaaaactgatttcagaaatgcatagttttatccagataaagaaaaaaacaaacccgagaaacaatgaccccaagagcatgaagctaccccaggaacagcgtcagcttccatatccttcagaggccagcacaaccttccctgagagccatcttcggactcctcagatgccaccaacaactccatccagcagtttcttcaccaagaaaagtgaagacacaatctccaaaatgaatgacttcatgaggatgcagatactgaaggaagggagtcattttccaggaccgttcatgaccagcataggcccagctgagagccatccccacactcctcagatgcctccatcaacaccaagcagcagtttcttaaccacgaaaagtgaagacacaatctccaaaatgaatgacttcatgaggatgcagatactgaaggaagggagtcattttccaggaccgttcatgaccagcataggcccagctgagagccatccccacactcctcagatgcctccatcaacaccaagcagcagtttcttaaccacgttgaaaccaagactgaagactgaacctgaagaagtttccatagaagacagtgcccagagtgacctcaaagaagtgatggtgctgaacgcaacagaatcatttgtatatgagcccaaagagcagaagaaaatgtttcatgccacagtggcaactgagaatgaagtcttccgagtgaaggtttttaatattgacctaaaggagaagttcaccccaaagaagatcattgccatagcaaattatgtttgccgcaatgggttcctggaggtatatcctttcacacttgtggctgatgtgaatgctgaccgaaacatggagatcccaaaaggattgattagaagtgccagcgtaactcctaaaatcaatcagctttgctcacaaactaaaggaagttttgtgaatggggtgtttgaggtacataagaaaaatgtaaggggtgaattcacttattatgaaatacaagataatacagggaagatggaagtggtggtgcatggacgactgaccacaatcaactgtgaggaaggagataaactgaaactcacctgctttgaattggcaccgaaaagtgggaataccggggagttgagatctgtaattcatagtcacatcaaggtcatcaagaccaggaaaaacaagaaagacatactcaatcctgattcaagtatggaaacttcaccagactttttcttctaa

SEQ ID NO: 65 - protein, 729 aa, Homo sapiens
MGKKYKNIVLLKGLEVINDYHFRMVKSLLSNDLKLNLKMREEYDKIQIADLMEEKFRGDAGLGKLIKIFEDIPTLEDLAETLKKEKLKVKGPALSRKRKKEVDATSPAPSTSSTVKTEGAEATPGAQNPKTVAKCQVTPRRNVLQKRPVIVKVLSTTKPFEYETPEMEKKIMFHATVATQTQFFHVKVLNTSLKEKFNGKKIIIISDYLEYDSLLEVNEESTVSEAGPNQTFEVPNKIINRAKETLKIDILHKQASGNIVYGVFMLHKKTVNQKTTIYEIQDDRGKMDVVGTGQCHNIPCEEGDKLQLFCFRLRKKNQMSKLISEMHSFIQIKKKTNPRNNDPKSMKLPQEQRQLPYPSEASTTFPESHLRTPQMPPTTPSSSFFTKKSEDTISKMNDFMRMQILKEGSHFPGPFMTSIGPAESHPHTPQMPPSTPSSSFLTTKSEDTISKMNDFMRMQILKEGSHFPGPFMTSIGPAESHPHTPQMPPSTPSSSFLTTLKPRLKTEPEEVSIEDSAQSDLKEVMVLNATESFVYEPKEQKKMFHATVATENEVFRVKVFNIDLKEKFTPKKIIAIANYVCRNGFLEVYPFTLVADVNADRNMEIPKGLIRSASVTPKINQLCSQTKGSFVNGVFEVHKKNVRGEFTYYEIQDNTGKMEVVVHGRLTTINCEEGDKLKLTCFELAPKSGNTGELRSVIHSHIKVIKTRKNKKDILNPDSSMETSPDFFF

SEQ ID NO: 66 - DNA, 1569 bp, Homo sapiens
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttaaaagaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 67 - protein, 522 aa, Homo sapiens
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 68 - DNA, 1869 bp, Homo sapiens
atggaggagtcggaacccgaacggaagcgggctcgcaccgacgaggtgcctgccggaggaagccgctccgaggcggaagatgaggacgacgaggactacgtgccctatgtgccgttacggcagcgccggcagctactgctccagaagctgctgcagcgaagacgcaagggagctgcggaggaagagcagcaggacagcggtagtgaaccccggggagatgaggacgacatcccgctaggccctcagtccaacgtcagcctcctggatcagcaccagcaccttaaagagaaggctgaagcgcgcaaagagtctgccaaggagaagcagctgaaggaagaagagaagatcctggagagtgttgccgagggccgagcattgatgtcagtgaaggagatggctaagggcattacgtatgatgaccccatcaaaaccagctggactccaccccgttatgttctgagcatgtctgaagagcgacatgagcgcgtgcggaagaaataccacatcctggtggagggagacggtatcccaccacccatcaagagcttcaaggaaatgaagtttcctgcagccatcctgagaggcctgaagaagaaaggcattcaccacccaacacccattcagatccagggcatccccaccattctatctggccgtgacatgataggcatcgctttcacgggttcaggcaagacactggtgttcacgttgcccgtcatcatgttctgcctggaacaagagaagaggttacccttctcaaagcgcgaggggccctatggactcatcatctgcccctcgcgggagctggcccggcagacccatggcatcctggagtactactgccgcctgctgcaggaggacagctcaccactcctgcgctgcgccctctgcattgggggcatgtccgtgaaagagcagatggagaccatccgacacggtgtacacatgatggtggccaccccggggcgcctcatggatttgctgcagaagaagatggtcagcctagacatctgtcgctacctggccctggacgaggctgaccgcatgatcgacatgggcttcgagggtgacatccgtaccatcttctcctacttcaagggccagcgacagaccctgctcttcagtgccaccatgccgaagaagattcagaactttgctaagagtgcccttgtaaagcctgtgaccatcaatgtggggcgcgctggggctgccagcctggatgtcatccaggaggtagaatatgtgaaggaggaggccaagatggtgtacctgctcgagtgcctgcagaagacacccccgcctgtactcatctttgcagagaagaaggcagacgtggacgccatccacgagtacctgctgctcaagggggttgaggccgtagccatccatgggggcaaagaccaggaggaacggactaaggccatcgaggcattccgggagggcaagaaggatgtcctagtagccacagacgttgcctccaagggcctggacttccctgccatccagcacgtcatcaattatgacatgccagaggagattgagaactatgtacaccggattggccgcaccgggcgctcgggaaacacaggcatcgccactaccttcatcaacaaagcgtgtgatgagtcagtgctgatggacctcaaagcgctgctgctagaagccaagcagaaggtgccgcccgtgctgcaggtgctgcattgcggggatgagtccatgctggacattggaggagagcgcggctgtgccttctgcgggggcctgggtcatcggatcactgactgccccaaactcgaggctatgcagaccaagcaggtcagcaacatcggtcgcaaggactacctggcccacagctccatggacttctga

SEQ ID NO: 69 - protein, 622 aa, Homo sapiens
MEESEPERKRARTDEVPAGGSRSEAEDEDDEDYVPYVPLRQRRQLLLQKLLQRRRKGAAEEEQQDSGSEPRGDEDDIPLGPQSNVSLLDQHQHLKEKAEARKESAKEKQLKEEEKILESVAEGRALMSVKEMAKGITYDDPIKTSWTPPRYVLSMSEERHERVRKKYHILVEGDGIPPPIKSFKEMKFPAAILRGLKKKGIHHPTPIQIQGIPTILSGRDMIGIAFTGSGKTLVFTLPVIMFCLEQEKRLPFSKREGPYGLIICPSRELARQTHGILEYYCRLLQEDSSPLLRCALCIGGMSVKEQMETIRHGVHMMVATPGRLMDLLQKKMVSLDICRYLALDEADRMIDMGFEGDIRTIFSYFKGQRQTLLFSATMPKKIQNFAKSALVKPVTINVGRAGAASLDVIQEVEYVKEEAKMVYLLECLQKTPPPVLIFAEKKADVDAIHEYLLLKGVEAVAIHGGKDQEERTKAIEAFREGKKDVLVATDVASKGLDFPAIQHVINYDMPEEIENYVHRIGRTGRSGNTGIATTFINKACDESVLMDLKALLLEAKQKVPPVLQVLHCGDESMLDIGGERGCAFCGGLGHRITDCPKLEAMQTKQVSNIGRKDYLAHSSMDF

SEQ ID NO: 70 - DNA, 1491 bp, Homo sapiens
atgtcagtgaaggagatggctaagggcattacgtatgatgaccccatcaaaaccagctggactccaccccgttatgttctgagcatgtctgaagagcgacatgagcgcgtgcggaagaaataccacatcctggtggagggagacggtatcccaccacccatcaagagcttcaaggaaatgaagtttcctgcagccatcctgagaggcctgaagaagaaaggcattcaccacccaacacccattcagatccagggcatccccaccattctatctggccgtgacatgataggcatcgctttcacgggttcaggcaagacactggtgttcacgttgcccgtcatcatgttctgcctggaacaagagaagaggttacccttctcaaagcgcgaggggccctatggactcatcatctgcccctcgcgggagctggcccggcagacccatggcatcctggagtactactgccgcctgctgcaggaggacagctcaccactcctgcgctgcgccctctgcattgggggcatgtccgtgaaagagcagatggagaccatccgacacggtgtacacatgatggtggccaccccggggcgcctcatggatttgctgcagaagaagatggtcagcctagacatctgtcgctacctggccctggacgaggctgaccgcatgatcgacatgggcttcgagggtgacatccgtaccatcttctcctacttcaagggccagcgacagaccctgctcttcagtgccaccatgccgaagaagattcagaactttgctaagagtgcccttgtaaagcctgtgaccatcaatgtggggcgcgctggggctgccagcctggatgtcatccaggaggtagaatatgtgaaggaggaggccaagatggtgtacctgctcgagtgcctgcagaagacacccccgcctgtactcatctttgcagagaagaaggcagacgtggacgccatccacgagtacctgctgctcaagggggttgaggccgtagccatccatgggggcaaagaccaggaggaacggactaaggccatcgaggcattccgggagggcaagaaggatgtcctagtagccacagacgttgcctccaagggcctggacttccctgccatccagcacgtcatcaattatgacatgccagaggagattgagaactatgtacaccggattggccgcaccgggcgctcgggaaacacaggcatcgccactaccttcatcaacaaagcgtgtgatgagtcagtgctgatggacctcaaagcgctgctgctagaagccaagcagaaggtgccgcccgtgctgcaggtgctgcattgcggggatgagtccatgctggacattggaggagagcgcggctgtgccttctgcgggggcctgggtcatcggatcactgactgccccaaactcgaggctatgcagaccaagcaggtcagcaacatcggtcgcaaggactacctggcccacagctccatggacttctga

SEQ ID NO: 71 - protein, 496 aa, Homo sapiens
MSVKEMAKGITYDDPIKTSWTPPRYVLSMSEERHERVRKKYHILVEGDGIPPPIKSFKEMKFPAAILRGLKKKGIHHPTPIQIQGIPTILSGRDMIGIAFTGSGKTLVFTLPVIMFCLEQEKRLPFSKREGPYGLIICPSRELARQTHGILEYYCRLLQEDSSPLLRCALCIGGMSVKEQMETIRHGVHMMVATPGRLMDLLQKKMVSLDICRYLALDEADRMIDMGFEGDIRTIFSYFKGQRQTLLFSATMPKKIQNFAKSALVKPVTINVGRAGAASLDVIQEVEYVKEEAKMVYLLECLQKTPPPVLIFAEKKADVDAIHEYLLLKGVEAVAIHGGKDQEERTKAIEAFREGKKDVLVATDVASKGLDFPAIQHVINYDMPEEIENYVHRIGRTGRSGNTGIATTFINKACDESVLMDLKALLLEAKQKVPPVLQVLHCGDESMLDIGGERGCAFCGGLGHRITDCPKLEAMQTKQVSNIGRKDYLAHSSMDF

SEQ ID NO: 72 - DNA, 2541 bp, Homo sapiens
atggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagaagtcatagttgggatgacaaaacatgtcaaaagtcagctaatgttagcagcatttggcataggaattactctcccagaccagagtcgggtactgtttcagatgccccacaattgaaggaaaatccaagtactgtgggagtggaacgagtgattagtactaaagggttaaatctcccaaggaaatcatccattgtgaaaagaccaagaagtgcagagctgtcagaagatgacctgttgagtcagtattctctttcatttacgaagaagaccaagaaaaatagctctgaaggcaataaatcattgagcttttctgaagtgtttgtgcctgacctggtaaatggacctactaacaaaaagagtgtaagcactccacctaggacgagaaataaatttgcaacatttttacaaaggaaaaatgaagaaagtggtgcagttgtggttccagggaccagaagcaggtttttttgcagttcagattctactgactgtgtatcaaacaaagtgagcatccagcctctggatgaaactgctgtcacagataaagagaacaatctgcatgaatcagagtatggagaccaagaaggcaagagactggttgacacagatgtagcacgtaattcaagtgatgacattccgaataatcatattccaggtgatcatattccagacaaggcaacagtgtttacagatgaagagtcctactcttttgagagcagcaaatttacaaggaccatttcaccacccactttgggaacactaagaagttgttttagttggtctggaggtcttggagatttttcaagaacgccgagcccctctccaagcacagcattgcagcagttccgaagaaagagcgattcccccacctctttgcctgagaataatatgtctgatgtgtcgcagttaaagagcgaggagtccagtgacgatgagtctcatcccttacgagaagaggcatgttcttcacagtcccaggaaagtggagaattctcactgcagagttcaaatgcatcaaagctttctcagtgctctagtaaggactctgattcagaggaatctgattgcaatattaagttacttgacagtcaaagtgaccagacctccaagctacgtttatctcatttctcaaaaaaagacacacctctaaggaacaaggttcctgggctatataagtccagttctgcagactctctttctacaaccaagatcaaacctctaggacctgccagagccagtgggctgagcaagaagccggcaagcatccagaagagaaagcatcataatgccgagaacaagccggggttacagatcaaactcaatgagctctggaaaaactttggatttaaaaaagattctgaaaagcttcctccttgtaagaaacccctgtccccagtcagagataacatccaactaactccagaagcggaagaggatatatttaacaaacctgaatgtggccgtgttcaaagagcaatattccagtaa

SEQ ID NO: 73 - protein, 846 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKDSEKLPPCKKPLSPVRDNIQLTPEAEEDIFNKPECGRVQRAIFQ

SEQ ID NO: 74 - DNA, 2538 bp, Homo sapiens
atggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagaagtcatagttgggatgacaaaacatgtcaaaagtcagctaatgttagcagcatttggcataggaattactctcccagaccagagtcgggtactgtttcagatgccccacaattgaaggaaaatccaagtactgtgggagtggaacgagtgattagtactaaagggttaaatctcccaaggaaatcatccattgtgaaaagaccaagaagtgagctgtcagaagatgacctgttgagtcagtattctctttcatttacgaagaagaccaagaaaaatagctctgaaggcaataaatcattgagcttttctgaagtgtttgtgcctgacctggtaaatggacctactaacaaaaagagtgtaagcactccacctaggacgagaaataaatttgcaacatttttacaaaggaaaaatgaagaaagtggtgcagttgtggttccagggaccagaagcaggtttttttgcagttcagattctactgactgtgtatcaaacaaagtgagcatccagcctctggatgaaactgctgtcacagataaagagaacaatctgcatgaatcagagtatggagaccaagaaggcaagagactggttgacacagatgtagcacgtaattcaagtgatgacattccgaataatcatattccaggtgatcatattccagacaaggcaacagtgtttacagatgaagagtcctactcttttgagagcagcaaatttacaaggaccatttcaccacccactttgggaacactaagaagttgttttagttggtctggaggtcttggagatttttcaagaacgccgagcccctctccaagcacagcattgcagcagttccgaagaaagagcgattcccccacctctttgcctgagaataatatgtctgatgtgtcgcagttaaagagcgaggagtccagtgacgatgagtctcatcccttacgagaagaggcatgttcttcacagtcccaggaaagtggagaattctcactgcagagttcaaatgcatcaaagctttctcagtgctctagtaaggactctgattcagaggaatctgattgcaatattaagttacttgacagtcaaagtgaccagacctccaagctacgtttatctcatttctcaaaaaaagacacacctctaaggaacaaggttcctgggctatataagtccagttctgcagactctctttctacaaccaagatcaaacctctaggacctgccagagccagtgggctgagcaagaagccggcaagcatccagaagagaaagcatcataatgccgagaacaagccggggttacagatcaaactcaatgagctctggaaaaactttggatttaaaaaagattctgaaaagcttcctccttgtaagaaacccctgtccccagtcagagataacatccaactaactccagaagcggaagaggatatatttaacaaacctgaatgtggccgtgttcaaagagcaatattccagtaa

SEQ ID NO: 75 - protein, 845 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKDSEKLPPCKKPLSPVRDNIQLTPEAEEDIFNKPECGRVQRAIFQ

SEQ ID NO: 76 - DNA, 2412 bp, Homo sapiens
atggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagaagtcatagttgggatgacaaaacatgtcaaaagtcagctaatgttagcagcatttggcataggaattactctcccagaccagagtcgggtactgtttcagatgccccacaattgaaggaaaatccaagtactgtgggagtggaacgagtgattagtactaaagggttaaatctcccaaggaaatcatccattgtgaaaagaccaagaagtgcagagctgtcagaagatgacctgttgagtcagtattctctttcatttacgaagaagaccaagaaaaatagctctgaaggcaataaatcattgagcttttctgaagtgtttgtgcctgacctggtaaatggacctactaacaaaaagagtgtaagcactccacctaggacgagaaataaatttgcaacatttttacaaaggaaaaatgaagaaagtggtgcagttgtggttccagggaccagaagcaggtttttttgcagttcagattctactgactgtgtatcaaacaaagtgagcatccagcctctggatgaaactgctgtcacagataaagagaacaatctgcatgaatcagagtatggagaccaagaaggcaagagactggttgacacagatgtagcacgtaattcaagtgatgacattccgaataatcatattccaggtgatcatattccagacaaggcaacagtgtttacagatgaagagtcctactcttttgagagcagcaaatttacaaggaccatttcaccacccactttgggaacactaagaagttgttttagttggtctggaggtcttggagatttttcaagaacgccgagcccctctccaagcacagcattgcagcagttccgaagaaagagcgattcccccacctctttgcctgagaataatatgtctgatgtgtcgcagttaaagagcgaggagtccagtgacgatgagtctcatcccttacgagaagaggcatgttcttcacagtcccaggaaagtggagaattctcactgcagagttcaaatgcatcaaagctttctcagtgctctagtaaggactctgattcagaggaatctgattgcaatattaagttacttgacagtcaaagtgaccagacctccaagctacgtttatctcatttctcaaaaaaagacacacctctaaggaacaaggttcctgggctatataagtccagttctgcagactctctttctacaaccaagatcaaacctctaggacctgccagagccagtgggctgagcaagaagccggcaagcatccagaagagaaagcatcataatgccgagaacaagccggggttacagatcaaactcaatgagctctggaaaaactttggatttaaaaaattctga

SEQ ID NO: 77 - protein, 803 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKF

SEQ ID NO: 78 - DNA, 3183 bp, Homo sapiens
atggagcagctgaacgaactggagctgctgatggagaagagtttttgggaggaggcggagctgccggcggagctatttcagaagaaagtggtagcttcctttccaagaacagttctgagcacaggaatggataaccggtacctggtgttggcagtcaatactgtacagaacaaagagggaaactgtgaaaagcgcctggtcatcactgcttcacagtcactagaaaataaagaactatgcatccttaggaatgactggtgttctgttccagtagagccaggagatatcattcatttggagggagactgcacatctgacacttggataatagataaagattttggatatttgattctgtatccagacatgctgatttctggcaccagcatagccagtagtattcgatgtatgagaagagctgtcctgagtgaaacttttaggagctctgatccagccacacgccaaatgctaattggtacggttctccatgaggtgtttcaaaaagccataaataatagctttgccccagaaaagctacaagaacttgcttttcaaacaattcaagaaataagacatttgaaggaaatgtaccgcttaaatctaagtcaagatgaaataaaacaagaagtagaggactatcttccttcgttttgtaaatgggcaggagatttcatgcataaaaacacttcgactgacttccctcagatgcagctctctctgccaagtgataatagtaaggataattcaacatgtaacattgaagtcgtgaaaccaatggatattgaagaaagcatttggtcccctaggtttggattgaaaggcaaaatagatgttacagttggtgtgaaaatacatcgagggtataaaacaaaatacaagataatgccgctggaacttaaaactggcaaagaatcaaattctattgaacaccgtagtcaggttgttctgtacactctactaagccaagagagaagagctgatccagaggctggcttgcttctctacctcaagactggtcagatgtaccctgtgcctgccaaccatctagataaaagagaattattaaagctaagaaaccagatggcattctcattgtttcaccgtattagcaaatctgctactagacagaagacacagcttgcttctttgccacaaataattgaggaagagaaaacttgtaaatattgttcacaaattggcaattgtgctctttatagcagagcagttgaacaacagatggattgtagttcagtcccaattgtgatgctgcccaaaatagaagaagaaacccagcatctgaagcaaacacacttagaatatttcagcctttggtgtctaatgttaaccctggagtcacaatcgaaggataataaaaagaatcaccaaaatatctggctaatgcctgcttcggaaatggagaagagtggcagttgcattggaaacctgattagaatggaacatgtaaagatagtttgtgatgggcaatatttacataatttccaatgtaaacatggtgccatacctgtcacaaatctaatggcaggtgacagagttattgtaagtggagaagaaaggtcactgtttgctttgtctagaggatatgtgaaggagattaacatgacaacagtaacttgtttattagacagaaacttgtcggtccttccagaatcaactttgttcagattagaccaagaagaaaaaaattgtgatatagataccccattaggaaatctttccaaattgatggaaaacacgtttgtcagcaaaaaacttcgagatttaattattgactttcgtgaacctcagtttatatcctaccttagttctgttcttccacatgatgcaaaggatacagttgcctgcattctaaagggtttgaataagcctcagaggcaagcgatgaaaaaggtacttctttcaaaagactacacactcatcgtgggtatgcctgggacaggaaaaacaactacgatatgtactctcgtaagaattctctacgcctgtggttttagcgttttgttgaccagctatacacactctgctgttgacaatattcttttgaagttagccaagtttaaaataggatttttgcgtttgggtcagattcagaaggttcatccagctatccagcaatttacagagcaagaaatttgcagatcaaagtccattaaatccttagctcttctagaagaactctacaatagtcaacttatagttgcaacaacatgtatgggaataaaccatccaatattttcccgtaaaatttttgatttttgtattgtggatgaagcctctcaaattagccaaccaatttgtctgggccccctttttttttcacggagatttgtgttagtgggggaccatcagcagcttcctcccctggtgctaaaccgtgaagcaagagctcttggcatgagtgaaagcttattcaagaggctggagcagaataagagtgctgttgtacagttaaccgtgcagtacagaatgaacagtaaaattatgtccttaagtaataagctgacctatgagggcaagctggagtgtggatcagacaaagtggccaatgcagtgataaacctacgtcactttaaagatgtgaagctggaactggaattttatgctgactattctgataatccttggttgatgggagtatttgaacccaacaatcctgtttgtttccttaatacagacaaggttccagcgccagaacaagttgaaaaaggtggtgtgagcaatgtaacagaagccaaactcatagttttcctaacctccatttttgttaaggctggatgcagtccctctgatattggtattattgcaccgtacaggcagcaattaaagatcatcaatgatttattggcacgttctattgggatggtcgaagttaatacagtagacaaataccaaggaagggacaaaagtattgtcctagtatcttttgttagaagtaataaggatggaactgttggtgaactcttgaaagattggcgacgtcttaatgttgctataaccagagccaaacataaactgattcttctggggtgtgtgccctcactaaattgctatcctcctttggagaagctgcttaatcatttaaactcagaaaaattaatcattgatcttccatcaagagaacatgaaagtctttgccacatattgggtgactttcaaagagaataa

SEQ ID NO: 79 - protein, 1060 aa, Homo sapiens
MEQLNELELLMEKSFWEEAELPAELFQKKVVASFPRTVLSTGMDNRYLVLAVNTVQNKEGNCEKRLVITASQSLENKELCILRNDWCSVPVEPGDIIHLEGDCTSDTWIIDKDFGYLILYPDMLISGTSIASSIRCMRRAVLSETFRSSDPATRQMLIGTVLHEVFQKAINNSFAPEKLQELAFQTIQEIRHLKEMYRLNLSQDEIKQEVEDYLPSFCKWAGDFMHKNTSTDFPQMQLSLPSDNSKDNSTCNIEVVKPMDIEESIWSPRFGLKGKIDVTVGVKIHRGYKTKYKIMPLELKTGKESNSIEHRSQVVLYTLLSQERRADPEAGLLLYLKTGQMYPVPANHLDKRELLKLRNQMAFSLFHRISKSATRQKTQLASLPQIIEEEKTCKYCSQIGNCALYSRAVEQQMDCSSVPIVMLPKIEEETQHLKQTHLEYFSLWCLMLTLESQSKDNKKNHQNIWLMPASEMEKSGSCIGNLIRMEHVKIVCDGQYLHNFQCKHGAIPVTNLMAGDRVIVSGEERSLFALSRGYVKEINMTTVTCLLDRNLSVLPESTLFRLDQEEKNCDIDTPLGNLSKLMENTFVSKKLRDLIIDFREPQFISYLSSVLPHDAKDTVACILKGLNKPQRQAMKKVLLSKDYTLIVGMPGTGKTTTICTLVRILYACGFSVLLTSYTHSAVDNILLKLAKFKIGFLRLGQIQKVHPAIQQFTEQEICRSKSIKSLALLEELYNSQLIVATTCMGINHPIFSRKIFDFCIVDEASQISQPICLGPLFFSRRFVLVGDHQQLPPLVLNREARALGMSESLFKRLEQNKSAVVQLTVQYRMNSKIMSLSNKLTYEGKLECGSDKVANAVINLRHFKDVKLELEFYADYSDNPWLMGVFEPNNPVCFLNTDKVPAPEQVEKGGVSNVTEAKLIVFLTSIFVKAGCSPSDIGIIAPYRQQLKIINDLLARSIGMVEVNTVDKYQGRDKSIVLVSFVRSNKDGTVGELLKDWRRLNVAITRAKHKLILLGCVPSLNCYPPLEKLLNHLNSEKLIIDLPSREHESLCHILGDFQRE

SEQ ID NO: 80 - DNA, 2694 bp, Homo sapiens
atgaacatctcgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaatctccaatggccaaagcacatggaacaagcagctatacccctgataagtcatcttttaatttagctacagttgttgctgaaacacttggacttggtgttcaagaagaatctgaaactcaaggtcccatgagcccccttggtgatgagctctaccactgtctggaaggaaatcacaagaaacagccttttgaggaatctacaagaaatactgaagatagtttaagattttcagattctacttcaaagactcctcctcaagaagaattacctactcgagtgtcatctcctgtatttggagctacctctagtatcaaaagtggtttagatttgaatacaagtttgtccccttctcttttacagcctgggaaaaaaaaacatctgaaaacactcccttttagcaacacttgtatatctagattagaaaaaactagatcaaaatctgaagatagtgcccttttcacacatcacagtcttgggtctgaagtgaacaagatcattatccagtcatctaataaacagatacttataaataaaaatataagtgaatccctaggtgaacagaataggactgagtacggtaaagattctaacactgataaacatttggagcccctgaaatcattgggaggccgaacatccaaaaggaagaaaactgaggaagaaagtgaacatgaagtaagctgcccccaagcttcttttgataaagaaaatgctttcccttttccaatggataatcagttttccatgaatggagactgtgtgatggataaacctctggatctgtctgatcgattttcagctattcagcgtcaagagaaaagccaaggaagtgagacttctaaaaacaaatttaggcaagtgactctttatgaggctttgaagaccattccaaagggcttttcctcaagccgtaaggcctcagatggcaactgcacgttgcccaaagattccccaggggagccctgttcacaggaatgcatcatccttcagcccttgaataaatgctctccagacaataaaccatcattacaaataaaagaagaaaatgctgtctttaaaattcctctacgtccacgtgaaagtttggagactgagaatgttttagatgacataaagagtgctggttctcatgagccaataaaaatacaaaccaggtcagaccatggaggatgtgaacttgcatcagttcttcagttaaatccatgtagaactggtaaaataaagtctctacaaaacaaccaagatgtatcctttgaaaatatccagtggagtatagatccgggagcagacctttctcagtataaaatggatgttactgtaatagatacaaaggatggcagtcagtcaaaattaggaggagagacagtggacatggactgtacattggttagtgaaaccgttctcttaaaaatgaagaagcaagagcagaagggagaaaaaagttcaaatgaagaaagaaaaatgaatgatagcttggaagatatgtttgatcggacaacacatgaagagtatgaatcctgtttggcagacagtttctcccaagcagcagatgaagaggaggaattgtctactgccacaaagaaactacacactcatggtgataaacaagacaaagtcaagcagaaagcgtttgtggagccgtattttaaaggtgatgaaagagagactagcttgcaaaattttcctcatattgaggtggttcggaaaaaagaggagagaagaaaactgcttgggcacacgtgtaaggaatgtgaaatttattatgcagatatgccagcagaagaaagagaaaagaaattggcttcctgctcaagacaccgattccgctacattccacccaacacaccagagaatttttgggaagttggttttccttccactcagacttgtatggaaagaggttatattaaggaagatcttgatccttgtcctcgtccaaaaagacgtcagccttacaacgcaatattttctccaaaaggcaaggagcagaagacatag

SEQ ID NO: 81 - protein, 897 aa, Homo sapiens
MNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQSPMAKAHGTSSYTPDKSSFNLATVVAETLGLGVQEESETQGPMSPLGDELYHCLEGNHKKQPFEESTRNTEDSLRFSDSTSKTPPQEELPTRVSSPVFGATSSIKSGLDLNTSLSPSLLQPGKKKHLKTLPFSNTCISRLEKTRSKSEDSALFTHHSLGSEVNKIIIQSSNKQILINKNISESLGEQNRTEYGKDSNTDKHLEPLKSLGGRTSKRKKTEEESEHEVSCPQASFDKENAFPFPMDNQFSMNGDCVMDKPLDLSDRFSAIQRQEKSQGSETSKNKFRQVTLYEALKTIPKGFSSSRKASDGNCTLPKDSPGEPCSQECIILQPLNKCSPDNKPSLQIKEENAVFKIPLRPRESLETENVLDDIKSAGSHEPIKIQTRSDHGGCELASVLQLNPCRTGKIKSLQNNQDVSFENIQWSIDPGADLSQYKMDVTVIDTKDGSQSKLGGETVDMDCTLVSETVLLKMKKQEQKGEKSSNEERKMNDSLEDMFDRTTHEEYESCLADSFSQAADEEEELSTATKKLHTHGDKQDKVKQKAFVEPYFKGDERETSLQNFPHIEVVRKKEERRKLLGHTCKECEIYYADMPAEEREKKLASCSRHRFRYIPPNTPENFWEVGFPSTQTCMERGYIKEDLDPCPRPKRRQPYNAIFSPKGKEQKT

SEQ ID NO: 82 - DNA, 2604 bp, Homo sapiens
atgaacatctcgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaatctccaatggccaaagcacatggaacaagcagctatacccctgataagtcatcttttaatttagctacagttgttgctgaaacacttggacttggtgttcaagaagaatctgaaactcaaggtcccatgagcccccttggtgatgagctctaccactgtctggaaggaaatcacaagaaacagccttttgaggaatctacaagaaatactgaagatagtttaagattttcagattctacttcaaagactcctcctcaagaagaattacctactcgagtgtcatctcctgtatttggagctacctctagtatcaaaagtggtttagatttgaatacaagtttgtccccttctcttttacagcctgggaaaaaaaaacatctgaaaacactcccttttagcaacacttgtatatctagattagaaaaaactagatcaaaatctgaagatagtgcccttttcacacatcacagtcttgggtctgaagtgaacaagatcattatccagtcatctaataaacagatacttataaataaaaatataagtgaatccctaggtgaacagaataggactgagtacggtaaagattctaacactgataaacatttggagcccctgaaatcattgggaggccgaacatccaaaaggaagaaaactgaggaagaaagtgaacatgaagtaagctgcccccaagcttcttttgataaagaaaatgctttcccttttccaatggataatcagttttccatgaatggagactgtgtgatggataaacctctggatctgtctgatcgattttcagctattcagcgtcaagagaaaagccaaggaagtgagacttctaaaaacaaatttaggcaagtgactctttatgaggctttgaagaccattccaaagggcttttcctcaagccgtaaggcctcagatggcaactgcacgttgcccaaagattccccaggggagccctgttcacaggaatgcatcatccttcagcccttgaataaatgctctccagacaataaaccatcattacaaataaaagaagaaaatgctgtctttaaaattcctctacgtccacgtgaaagtttggagactgagaatgttttagatgacataaagagtgctggttctcatgagccaataaaaatacaaaccaggtcagaccatggaggatgtgaacttgcatcagttcttcagttaaatccatgtagaactggtaaaataaagtctctacaaaacaaccaagatgtatcctttgaaaatatccagtggagtatagatccgggagcagacctttctcagtataaaatggatgttactgtaatagatacaaaggatggcagtcagtcaaaattaggaggagagacagtggacatggactgtacattggttagtgaaaccgttctcttaaaaatgaagaagcaagagcagaagggagaaaaaagttcaaatgaagaaagaaaaatgaatgatagcttggaagatatgtttgatcggacaacacatgaagagtatgaatcctgtttggcagacagtttctcccaagcagcagatgaagaggaggaattgtctactgccacaaagaaactacacactcatggtgataaacaagacaaagtcaagcagaaagcgtttgtggagccgtattttaaaggtgatgaaagtattatgcagatatgccagcagaagaaagagaaaagaaattggcttcctgctcaagacaccgattccgctacattccacccaacacaccagagaatttttgggaagttggttttccttccactcagacttgtatggaaagaggttatattaaggaagatcttgatccttgtcctcgtccaaaaagacgtcagccttacaacgcaatattttctccaaaaggcaaggagcagaagacatagacgttga

SEQ ID NO: 83 - protein, 867 aa, Homo sapiens
MNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQSPMAKAHGTSSYTPDKSSFNLATVVAETLGLGVQEESETQGPMSPLGDELYHCLEGNHKKQPFEESTRNTEDSLRFSDSTSKTPPQEELPTRVSSPVFGATSSIKSGLDLNTSLSPSLLQPGKKKHLKTLPFSNTCISRLEKTRSKSEDSALFTHHSLGSEVNKIIIQSSNKQILINKNISESLGEQNRTEYGKDSNTDKHLEPLKSLGGRTSKRKKTEEESEHEVSCPQASFDKENAFPFPMDNQFSMNGDCVMDKPLDLSDRFSAIQRQEKSQGSETSKNKFRQVTLYEALKTIPKGFSSSRKASDGNCTLPKDSPGEPCSQECIILQPLNKCSPDNKPSLQIKEENAVFKIPLRPRESLETENVLDDIKSAGSHEPIKIQTRSDHGGCELASVLQLNPCRTGKIKSLQNNQDVSFENIQWSIDPGADLSQYKMDVTVIDTKDGSQSKLGGETVDMDCTLVSETVLLKMKKQEQKGEKSSNEERKMNDSLEDMFDRTTHEEYESCLADSFSQAADEEEELSTATKKLHTHGDKQDKVKQKAFVEPYFKGDESIMQICQQKKEKRNWLPAQDTDSATFHPTHQRIFGKLVFLPLRLVWKEVILRKILILVLVQKDVSLTTQYFLQKARSRRHRR

SEQ ID NO: 84 - DNA, 2127 bp, Homo sapiens
atgagtactgcagatgcacttgatgatgaaaacacatttaaaatattagttgcaacagatattcatcttggatttatggagaaagatgcagtcagaggaaatgatacgtttgtaacactcgatgaaattttaagacttgcccaggaaaatgaagtggattttattttgttaggtggtgatctttttcatgaaaataagccctcaaggaaaacattacatacctgcctcgagttattaagaaaatattgtatgggtgatcggcctgtccagtttgaaattctcagtgatcagtcagtcaactttggttttagtaagtttccatgggtgaactatcaagatggcaacctcaacatttcaattccagtgtttagtattcatggcaatcatgacgatcccacaggggcagatgcactttgtgccttggacattttaagttgtgctggatttgtaaatcactttggacgttcaatgtctgtggagaagatagacattagtccggttttgcttcaaaaaggaagcacaaagattgcgctatatggtttaggatccattccagatgaaaggctctatcgaatgtttgtcaataaaaaagtaacaatgttgagaccaaaggaagatgagaactcttggtttaacttatttgtgattcatcagaacaggagtaaacatggaagtactaacttcattccagaacaatttttggatgacttcattgatcttgttatctggggccatgaacatgagtgtaaaatagctccaaccaaaaatgaacaacagctgttttatatctcacaacctggaagctcagtggttacttctctttccccaggagaagctgtaaagaaacatgttggtttgctgcgtattaaagggaggaagatgaatatgcataaaattcctcttcacacagtgcggcagtttttcatggaggatattgttctagctaatcatccagacatttttaacccagataatcctaaagtaacccaagccatacaaagcttctgtttggagaagattgaagaaatgcttgaaaatgctgaacgggaacgtctgggtaattctcaccagccagagaagcctcttgtacgactgcgagtggactatagtggaggttttgaacctttcagtgttcttcgctttagccagaaatttgtggatcgggtagctaatccaaaagacattatccattttttcaggcatagagaacaaaaggaaaaaacaggagaagagatcaactttgggaaacttatcacaaagccttcagaaggaacaactttaagggtagaagatcttgtaaaacagtactttcaaaccgcagagaagaatgtgcagctctcactgctaacagaaagagggatgggtgaagcagtacaagaatttgtggacaaggaggagaaagatgccattgaggaattagtgaaataccagttggaaaaaacacagcgatttcttaaagaacgtcatattgatgccctcgaagacaaaatcgatgaggaggtacgtcgtttcagagaaaccagacaaaaaaatactaatgaagaagatgatgaagtccgtgaggctatgaccagggccagagcactcagatctcagtcagaggagtctgcttctgcctttagtgctgatgaccttatgagtatagatttagcagaacagatggctaatgactctgatgatagcatctcagcagcaaccaacaaaggaagaggccgaggaagaggtcgaagaggtggaagagggcagaattcagcatcgagaggagggtctcaaagaggaagagcagacactggtctggagacttctacccgtagcaggaactcaaagactgctgtgtcagcatctagaaatatgtctattatagatgcctttaaatctacaagacagcagccttcccgaaatgtcactactaagaattattcagaggtgattgaggtagatgaatcagatgtggaagaagacatttttcctaccacttcaaagacagatcaaaggtggtccagcacatcatccagcaaaatcatgtcccagagtcaagtatcgaaaggggttgattttgaatcaagtgaggatgatgatgatgatccttttatgaacactagttctttaagaagaaatagaagataa

SEQ ID NO: 85 - protein, 708 aa, Homo sapiens
MSTADALDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKEDENSWFNLFVIHQNRSKHGSTNFIPEQFLDDFIDLVIWGHEHECKIAPTKNEQQLFYISQPGSSVVTSLSPGEAVKKHVGLLRIKGRKMNMHKIPLHTVRQFFMEDIVLANHPDIFNPDNPKVTQAIQSFCLEKIEEMLENAERERLGNSHQPEKPLVRLRVDYSGGFEPFSVLRFSQKFVDRVANPKDIIHFFRHREQKEKTGEEINFGKLITKPSEGTTLRVEDLVKQYFQTAEKNVQLSLLTERGMGEAVQEFVDKEEKDAIEELVKYQLEKTQRFLKERHIDALEDKIDEEVRRFRETRQKNTNEEDDEVREAMTRARALRSQSEESASAFSADDLMSIDLAEQMANDSDDSISAATNKGRGRGRGRRGGRGQNSASRGGSQRGRADTGLETSTRSRNSKTAVSASRNMSIIDAFKSTRQQPSRNVTTKNYSEVIEVDESDVEEDIFPTTSKTDQRWSSTSSSKIMSQSQVSKGVDFESSEDDDDDPFMNTSSLRRNRR

SEQ ID NO: 86 - DNA, 2043 bp, Homo sapiens
atgagtactgcagatgcacttgatgatgaaaacacatttaaaatattagttgcaacagatattcatcttggatttatggagaaagatgcagtcagaggaaatgatacgtttgtaacactcgatgaaattttaagacttgcccaggaaaatgaagtggattttattttgttaggtggtgatctttttcatgaaaataagccctcaaggaaaacattacatacctgcctcgagttattaagaaaatattgtatgggtgatcggcctgtccagtttgaaattctcagtgatcagtcagtcaactttggttttagtaagtttccatgggtgaactatcaagatggcaacctcaacatttcaattccagtgtttagtattcatggcaatcatgacgatcccacaggggcagatgcactttgtgccttggacattttaagttgtgctggatttgtaaatcactttggacgttcaatgtctgtggagaagatagacattagtccggttttgcttcaaaaaggaagcacaaagattgcgctatatggtttaggatccattccagatgaaaggctctatcgaatgtttgtcaataaaaaagtaacaatgttgagaccaaaggaagatgagaactcttggtttaacttatttgtgattcatcagaacaggagtaaacatggaagtactaacttcattccagaacaatttttggatgacttcattgatcttgttatctggggccatgaacatgagtgtaaaatagctccaaccaaaaatgaacaacagctgttttatatctcacaacctggaagctcagtggttacttctctttccccaggagaagctgtaaagaaacatgttggtttgctgcgtattaaagggaggaagatgaatatgcataaaattcctcttcacacagtgcggcagtttttcatggaggatattgttctagctaatcatccagacatttttaacccagataatcctaaagtaacccaagccatacaaagcttctgtttggagaagattgaagaaatgcttgaaaatgctgaacgggaacgtctgggtaattctcaccagccagagaagcctcttgtacgactgcgagtggactatagtggaggttttgaacctttcagtgttcttcgctttagccagaaatttgtggatcgggtagctaatccaaaagacattatccattttttcaggcatagagaacaaaaggaaaaaacaggagaagagatcaactttgggaaacttatcacaaagccttcagaaggaacaactttaagggtagaagatcttgtaaaacagtactttcaaaccgcagagaagaatgtgcagctctcactgctaacagaaagagggatgggtgaagcagtacaagaatttgtggacaaggaggagaaagatgccattgaggaattagtgaaataccagttggaaaaaacacagcgatttcttaaagaacgtcatattgatgccctcgaagacaaaatcgatgaggaggtacgtcgtttcagagaaaccagacaaaaaaatactaatgaagaagatgatgaagtccgtgaggctatgaccagggccagagcactcagatctcagtcagaggagtctgcttctgcctttagtgctgatgaccttatgagtatagatttagcagaacagatggctaatgactctgatgatagcatctcagcagcaaccaacaaaggaagaggccgaggaagaggtcgaagaggtggaagagggcagaattcagcatcgagaggagggtctcaaagaggaagagcctttaaatctacaagacagcagccttcccgaaatgtcactactaagaattattcagaggtgattgaggtagatgaatcagatgtggaagaagacatttttcctaccacttcaaagacagatcaaaggtggtccagcacatcatccagcaaaatcatgtcccagagtcaagtatcgaaaggggttgattttgaatcaagtgaggatgatgatgatgatccttttatgaacactagttctttaagaagaaatagaagataa

SEQ ID NO: 87 - protein, 680 aa, Homo sapiens
MSTADALDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKEDENSWFNLFVIHQNRSKHGSTNFIPEQFLDDFIDLVIWGHEHECKIAPTKNEQQLFYISQPGSSVVTSLSPGEAVKKHVGLLRIKGRKMNMHKIPLHTVRQFFMEDIVLANHPDIFNPDNPKVTQAIQSFCLEKIEEMLENAERERLGNSHQPEKPLVRLRVDYSGGFEPFSVLRFSQKFVDRVANPKDIIHFFRHREQKEKTGEEINFGKLITKPSEGTTLRVEDLVKQYFQTAEKNVQLSLLTERGMGEAVQEFVDKEEKDAIEELVKYQLEKTQRFLKERHIDALEDKIDEEVRRFRETRQKNTNEEDDEVREAMTRARALRSQSEESASAFSADDLMSIDLAEQMANDSDDSISAATNKGRGRGRGRRGGRGQNSASRGGSQRGRAFKSTRQQPSRNVTTKNYSEVIEVDESDVEEDIFPTTSKTDQRWSSTSSSKIMSQSQVSKGVDFESSEDDDDDPFMNTSSLRRNRR

SEQ ID NO: 88 - DNA, 2124 bp, Homo sapiens
atgagtactgcagatgcacttgatgatgaaaacacatttaaaatattagttgcaacagatattcatcttggatttatggagaaagatgcagtcagaggaaatgatacgtttgtaacactcgatgaaattttaagacttgcccaggaaaatgaagtggattttattttgttaggtggtgatctttttcatgaaaataagccctcaaggaaaacattacatacctgcctcgagttattaagaaaatattgtatgggtgatcggcctgtccagtttgaaattctcagtgatcagtcagtcaactttggttttagtaagtttccatgggtgaactatcaagatggcaacctcaacatttcaattccagtgtttagtattcatggcaatcatgacgatcccacaggggcagatgcactttgtgccttggacattttaagttgtgctggatttgtaaatcactttggacgttcaatgtctgtggagaagatagacattagtccggttttgcttcaaaaaggaagcacaaagattgcgctatatggtttaggatccattccagatgaaaggctctatcgaatgtttgtcaataaaaaagtaacaatgttgagaccaaaggaagatgagaactcttggtttaacttatttgtgattcatcagaacaggagtaaacatggaagtactaacttcattccagaacaatttttggatgacttcattgatcttgttatctggggccatgaacatgagtgtaaaatagctccaaccaaaaatgaacaacagctgttttatatctcacaacctggaagctcagtggttacttctctttccccaggagaagctgtaaagaaacatgttggtttgctgcgtattaaagggaggaagatgaatatgcataaaattcctcttcacacagtgcggcagtttttcatggaggatattgttctagctaatcatccagacatttttaacccagataatcctaaagtaacccaagccatacaaagcttctgtttggagaagattgaagaaatgcttgaaaatgctgaacgggaacgtctgggtaattctcaccagccagagaagcctcttgtacgactgcgagtggactatagtggaggttttgaacctttcagtgttcttcgctttagccagaaatttgtggatcgggtagctaatccaaaagacattatccattttttcaggcatagagaacaaaaggaaaaaacaggagaagagatcaactttgggaaacttatcacaaagccttcagaaggaacaactttaagggtagaagatcttgtaaaacagtactttcaaaccgcagagaagaatgtgcagctctcactgctaacagaaagagggatgggtgaagcagtacaagaatttgtggacaaggaggagaaagatgccattgaggaattagtgaaataccagttggaaaaaacacagcgatttcttaaagaacgtcatattgatgccctcgaagacaaaatcgatgaggaggtacgtcgtttcagagaaaccagacaaaaaaatactaatgaagaagatgatgaagtccgtgaggctatgaccagggccagagcactcagatctcagtcagaggagtctgcttctgcctttagtgctgatgaccttatgagtatagatttagcagaacagatggctaatgactctgatgatagcatctcagcagcaaccaacaaaggaagaggccgaggaagaggtcgaagaggtggaagagggcagaattcagcatcgagaggagggtctcaaagaggaagagacactggtctggagacttctacccgtagcaggaactcaaagactgctgtgtcagcatctagaaatatgtctattatagatgcctttaaatctacaagacagcagccttcccgaaatgtcactactaagaattattcagaggtgattgaggtagatgaatcagatgtggaagaagacatttttcctaccacttcaaagacagatcaaaggtggtccagcacatcatccagcaaaatcatgtcccagagtcaagtatcgaaaggggttgattttgaatcaagtgaggatgatgatgatgatccttttatgaacactagttctttaagaagaaatagaagataa

SEQ ID NO: 89 - protein, 707 aa, Homo sapiens
MSTADALDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKEDENSWFNLFVIHQNRSKHGSTNFIPEQFLDDFIDLVIWGHEHECKIAPTKNEQQLFYISQPGSSVVTSLSPGEAVKKHVGLLRIKGRKMNMHKIPLHTVRQFFMEDIVLANHPDIFNPDNPKVTQAIQSFCLEKIEEMLENAERERLGNSHQPEKPLVRLRVDYSGGFEPFSVLRFSQKFVDRVANPKDIIHFFRHREQKEKTGEEINFGKLITKPSEGTTLRVEDLVKQYFQTAEKNVQLSLLTERGMGEAVQEFVDKEEKDAIEELVKYQLEKTQRFLKERHIDALEDKIDEEVRRFRETRQKNTNEEDDEVREAMTRARALRSQSEESASAFSADDLMSIDLAEQMANDSDDSISAATNKGRGRGRGRRGGRGQNSASRGGSQRGRDTGLETSTRSRNSKTAVSASRNMSIIDAFKSTRQQPSRNVTTKNYSEVIEVDESDVEEDIFPTTSKTDQRWSSTSSSKIMSQSQVSKGVDFESSEDDDDDPFMNTSSLRRNRR